(19) United States
(12) Patent Application Publication
(10) Pub. No.: US 2024/0167026 A1
(43) Pub. Date: May 23, 2024

(54) GENOME EDITING COMPOSITIONS AND METHODS FOR TREATMENT OF WILSON'S DISEASE

(71) Applicant: PRIME MEDICINE, INC., Cambridge, MA (US)

(72) Inventors: Jonathan M. LEVY, Brookline, MA (US); Wei Hsi YEH, Cambridge, MA (US); Aaron Nakwon CHANG, Lexington, MA (US); John STILLER, Cambridge, MA (US)

(21) Appl. No.: 18/526,247

(22) Filed: Dec. 1, 2023

Related U.S. Application Data
(63) Continuation of application No. PCT/US2022/032267, filed on Jun. 3, 2022.
(60) Provisional application No. 63/196,380, filed on Jun. 3, 2021.

(51) Int. Cl. C12N 15/11

(52) U.S. Cl. CPC C12N 15/11; C12N 2310/20

(57) ABSTRACT

Provided herein are compositions and methods of using prime editing systems comprising prime editors and prime editing guide RNAs for treatment of genetic disorders.

### CROSS-REFERENCE

This application is a continuation of International Application No. PCT/US2022/032267, filed Jun. 3, 2022, which claims the benefit of U.S. Provisional Application No. 63/196,380, filed Jun. 3, 2021, each of which applications are incorporated herein by reference in its entirety.

### SEQUENCE LISTING

The instant application contains a Sequence Listing which has been submitted electronically in XML format and is hereby incorporated by reference in its entirety. Said XML copy, created on Nov. 30, 2023, is named 59761-721.301 Sequence Listing.xml and is 8,280,972 bytes in size.

### BACKGROUND

Wilson's disease is an autosomal recessive genetic copper storage disorder caused by mutations in the ATP7B gene (OMIM #606882). ATP7B is located in the human genome on 13q14.3 and contains 20 introns and 21 exons, for a total genomic length of about 80 kb. The ATP7B gene encodes ATPase copper transporting beta (ATP7B), a P-type transmembrane copper-transporting ATPase, which is mainly expressed in hepatic and neural tissues and functions in the transmembrane transport of copper. ATP7B deficiencies may lead to decreased hepatocellular excretion of copper into bile that may lead to systemic copper buildup primarily in the liver and subsequently in the neurologic system and other tissues, hepatic and neural toxicity, and early demise. The accumulation of copper can be manifested as neurological or psychiatric symptom. Over time without proper treatments, high copper levels can cause life-threatening organ damage. Failure to incorporate copper into ceruloplasmin is an additional consequence of the loss of functional ATP7B protein.

Current treatment approaches for Wilson's disease are daily oral therapy with chelating agents (such as penicillamine [Cuprimine] and trientine hydrochloride [Syprine]), zinc (to block enterocyte absorption of copper), and tetrathiomolybdate (TM), a copper chelator that forms complexes with albumin in the circulation; all of which require the affected individual to take medicines for their whole life. Furthermore, those treatments may cause side effects, such as drug induced lupus, myasthenia, paradoxical worsening, and do not restore normal copper metabolism. Liver transplantation is curative for Wilson's disease but transplant recipients are required to maintain a constant immune suppression regimen to prevent rejection. Therapeutic strategies, such as gene therapy, that can reverse the underlying metabolic defect would be greatly advantageous. However, the ATP7B gene is approximately 4.4 kb, nearing the adeno-associated virus (AAV) packaging size limit and making gene therapy approaches with the full-length gene difficult.

This disclosure provides Prime Editing methods and compositions for correcting mutations associated with Wilson's disease.

### SUMMARY OF THE INVENTION

Provided herein, in some embodiments, are methods and compositions for prime editing of alterations in a target sequence in a target gene, for example, an ATP7B gene. The target ATP7B gene may comprise double stranded DNA. As exemplified in FIG. 1, in an embodiment, the target gene is edited by prime editing.

Without wishing to be bound by any particular theory, the prime editing process may search specific targets and edit endogenous sequences in a target gene, e.g., the ATP7B gene. As exemplified in FIG. 1, the spacer sequence of a PEgRNA recognizes and anneals with a search target sequence in a target strand of the target gene. A prime editing complex may generate a nick in the target gene on the edit strand which is the complementary strand of the target strand. The prime editing complex may then use a free 3′ end formed at the nick site of the edit strand to initiate DNA synthesis, where a primer binding site (PBS) of the PEgRNA complexes with the free 3′ end, and a single stranded DNA is synthesized using an editing template of the PEgRNA as a template. The editing template may comprise one or more nucleotide edits compared to the endogenous target ATP7B gene sequence. Accordingly, the newly-synthesized single stranded DNA also comprises the nucleotide edit(s) encoded by the editing template. Through removal of an editing target sequence on the edit strand of the target gene and DNA repair, the intended nucleotide edit(s) included in the newly synthesized single stranded DNA are incorporated into the target ATP7B gene.

One embodiment of the disclosure provides a prime editing guide RNA (PEgRNA) comprising: (a) a spacer that is complementary to a search target sequence on a first strand of an ATP7B gene, wherein the spacer comprises at its 3′ end nucleotides 5-20 of SEQ ID NO: 4425; (b) a gRNA core capable of binding to a Cas9 protein; (c) an extension arm comprising: (i) an editing template that comprises a region of complementarity to an editing target sequence on a second strand of the ATP7B gene, and (ii) a primer binding site that comprises at its 5′ end a sequence that is a reverse complement of nucleotides 15-17 of SEQ ID NO: 4425; wherein the first strand and second strand are complementary to each other and wherein the editing target sequence on the second strand is complementary to a portion of the ATP7B gene comprising a c.3207C>A substitution.

Another embodiment of the disclosure provides a prime editing guide RNA (PEgRNA) comprising: (a) a spacer comprising at its 3′ end nucleotides 5-20 of SEQ ID NO: 4425; (b) a gRNA core capable of binding to a Cas9 protein, and (c) an extension an comprising: (i) an editing template comprising at its 3′ end any one of SEQ ID NOs: 4437-4492, and (ii) a primer binding site (PBS) sequence comprising at its 5′ end any one of SEQ ID NOs: 2297, 4426, 4427, 4428, 4429, 4430, 4431, 4432, 4433, 4434, 4435, and 4436.

In some embodiments, the spacer of the PEgRNA is front 15 to 22 nucleotides in length. In some embodiments, the spacer of the PEgRNA comprises at its 3′ end nucleotides 4-20, 3-20, 2-20, or 1-20 of SEQ ID NO: 4425. In some embodiments, the spacer of the PEgRNA comprises at its 3′ end SEQ ID NO: 4425. In some embodiments, the spacer of the PEgRNA is 20 nucleotides in length. In some embodiments, the PEgRNA of the present disclosure, comprises from 5′ to 3′, the spacer, the gRNA core, the RTT, and the PBS. In some embodiments, the spacer, the gRNA core, the RTT, and the PBS form a contiguous sequence in a single molecule.

In some embodiments, the PEgRNA of the present disclosure comprises a pegRNA sequence selected from any one of SEQ ID NOs: 2445, 2446, 2447, 2448, 2449, 2450, 2451, 2452, 2453, 2454, 2455, 2456, 2457, 2458, 2459, 2460, 2461, 2462, 2463, 2464, 2465, 2466, 2467, 2468, 2469, 2470, 2471, 2472, 2473, 2474, 2475, 2476, 2477, 2478, 2479, 2480, 2481, 2482, 2483, 2484, 2485, 2486, 2487, 2488, 2489, 2490, 2491, 2492, 2493, 2494, 2495, 2496, 2497, 2498, 2499, 2500, 2501, 2502, 2503, 2504, 2505, 2506, 2507, 2508, 2509, 2510, 2511, 2512, 2513, 2514, 2515, 2516, 2517, 2518, 2519, 2520, 2521, 2522, 2523, 2524, 2525, 2526, 2527, 2528, 2529, 2530, 2531, 2532, 2533, 2534, 2535, 2538, 2539, 2540, 2541, 2542, 2543, 2544, 2545, 2546, 2547, 2548, 2549, 2550, 2551, 2553, 2554, 2555, 2556, 2557, 2558, 2559, 2560, 2561, 2562, 2563, 2564, 2565, 2566, 2567, 2568, 2569, 2570, 2571, 2572, 2573, 2574, 2575, 2576, 2577, 2578, 2580, 2582, 2584, 2585, 2586, 2587, 2588, 2589, 2590, 2591, 2592, 2593, 2594, 2595, 2596, 2597, 2598, 2600, 2601, 2602, 2603, 2605, 2606, 2607, 2608, 2609, 2610, 2611, 2612, 2613, 2614, 2615, 2616, 2617, 2618, 2619, 2620, 2621, 2623, 2624, 2628, 2629, 2630, 2631, 2632, 2633, 2634, 2635, 2636, 2637, 2638, 2643, 2645, 2646, 2647, 2648, 2649, 2650, 2651, 2652, 2653, 2654, 2655, 2656, 2657, 2658, 2659, 2660, 2663, 2664, 2665, 2667, 2668, 2669, 2670, 2671, 2672, 2674, 2675, 2676, 2677, 2678, 2680, 2681, 2683, 2685, 2687, 2688, 2689, 2690, 2692, 2694, 2695, 2696, 2697, 2699, 2701, 2702, 2704, 2706, 2708, 2711, 2713, 2715, 2716, 2717, 2720, 2721, 2722, 2723, 2725, 2726, 2727, 2728, 2729, 2730, 2733, 2734, 2735, 2744, 2747, 2748, 2749, 2752, 2753, 2757, 2758, 2759, 2760, 2761, 2762, 2764, 2765, 2768, 2769, 2770, 2772, 2773, 2774, 2777, 2786, 2788, 2789, 2790, 2791, 2792, 2793, 2794, 2795, 2796, 2797, 2798, 2799, 2800, 2801, 2802, 2807, 2810, 2811, 2812, 2814, 2816, 2824, 2825, 2826, 2828, 2829, 2830, 2832, 2833, 2834, 2841, 2842, 2843, 2844, 2846, 2847, 2854, 2855, 2856, 2857, 2862, 2864, 2866, 2867, 2868, 2869, 2870, 2871, 2885, 2886, 2887, 2888, 2889, 2890, 2891, 2893, 2894, 2896, 2898, 2899, 2901, 2902, 2909, 2910, 2914, 2916, 2918, 2919, 2920, 2926, 2927, 2932, 2933, 2937, 2938, 2939, 2941, 2942, 2945, 2953, 2954, 2956, 2957, 2960, 2962, 2963, 2964, 2965, 2967, 2972, 2973, 2977, 2979, 2980, 2982, 2983, 2988, 2991, 2993, 2994, 2995, 2997, 3006, 3008, 3012, 3013, 3015, 3023, 3024, 3027, 3028, 3029, 3030, 3031, 3032, 3033, 3034, 3035, 3036, 3037, 3038, 3039, 3043, 3044, 3045, 3046, 3048, 3049, 3050, 3051, 3052, 3053, 3054, 3055, 3059, 3064, 3065, 3071, 3072, 3075, 3076, 3080, 3082, 3084, 3093, 3096, 3098, 3099, 3101, 3119, 3121, 3122, 3123, 3124, 3126, 3128, 3130, 3133, 3142, 3144, 3148, 3159, 3161, 3162, 3163, 3164, 3165, 3166, 3168, 3169, 3170, 3176, 3182, 3188, 3190, 3191, 3195, 3200, 3202, 3203, 3210, 3212, 3216, 3218, 3226, 3227, 3228, 3229, 3230, 3231, 3232, 3234, 3235, 3238, 3239, 3241, 3243, 3244, 3245, 3246, 3247, 3248, 3249, 3250, 3260, 3262, 3263, 3271, 3273, 3275, 3281, 3282, 3283, 3287, 3288, 3289, 3300, 3301, 3302, 3303, 3304, 3305, 3307, 3310, 3311, 3312, 3313, 3314, 3315, 3316, 3317, 3318, 3322, 3324, 3325, 3328, 3330, 3346, 3347, 3348, 3349, 3350, 3358, 3359, 3362, 3364, 3365, 3366, 3367, 3368, 3372, 3373, 3382, 3385, 3387, 3388, 3389, 3390, 3391, 3392, 3393, 3400, 3403, 3404, 3405, 3407, 3408, 3409, 3412, 3414, 3420, 3423, 3425, 3426, 3427, 3428, 3429, 3430, 3431, 3434, 3438, 3441, 3442, 3446, 3449, 3450, 3451, 3452, 3453, 3454, 3455, 3463, 3466, 3469, 3470, 3471, 3472, 3473, 3474, 3477, 3478, 3480, 3481, 3482, 3487, 3490, 3494, 3498, 3499, 3502, 3503, 3505, 3506, 3508, 3509, 3510, 3511, 3513, 3520, 3522, 3523, 3526, 3529, 3533, 3535, 3536, 3542, 3543, 3546, 3547, 3549, 3550, 3553, 3554, 3555, 3557, 3560, 3561, 3563, 3564, 3567, 3568, 3569, 3571, 3574, 3575, 3576, 3578, 3579, 3580, 3581, 3583, 3584, 3585, 3592, 3594, 3595, 3596, 3597, 3603, 3612, 3613, 3617, 3622, 3625, 3626, 3627, 3628, 3630, 3631, 3632, 3633, 3635, 3636, 3638, 3639, 3640, 3641, 3642, 3646, 3647, 3648, 3654, 3657, 3659, 3660, 3661, 3664, 3668, 3669, 3673, 3674, 3678, 3679, 3680, 3681, 3684, 3685, 3687, 3688, 3697, 3699, 3702, 3703, 3704, 3705, 3706, 3708, 3710, 3711, 3712, 3714, 3715, 3721, 3722, 3724, 3725, 3728, 3729, 3730, 3731, 3732, 3733, 3734, 3735, 3736, 3737, 3739, 3740, 3741, 3743, 3744, 3746, 3748, 3755, 3761, 3770, 3771, 3773, 3774, 3776, 3778, 3779, 3781, 3782, 3784, 3785, 3792, 3793, 3794, 3795, 3796, 3797, 3798, 3799, 3800, 3801, 3802, 3803, 3804, 3805, 3806, 3807, 3808, 3809, 3810, 3811, 3812, 3814, 3815, 3816, 3820, 3829, 3839, 3841, 3842, 3843, 3844, 3845, 3851, 3852, 3853, 3854, 3855, 3856, 3857, 3858, 3859, 3860, 3861, 3862, 3863, 3864, 3865, 3868, 3869, 3871, 3874, 3875, 3876, 3877, 3878, 3879, 3880, 3882, 3883, 3884, 3885, 3887, 3895, 3899, 3904, 3907, 3908, 3909, 3910, 3911, 3912, 3913, 3914, 3915, 3916, 3917, 3921, 3924, 3927, 3928, 3929, 3931, 3932, 3935, 3936, 3937, 3938, 3939, 3940, 3941, 3942, 3943, 3945, 3946, 3956, 3957, 3961, 3962, 3965, 3971, 3977, 3978, 3979, 3980, 3981, 3982, 3983, 3985, 3988, 3989, 3990, 3991, 3992, 3993, 3994, 3995, 3997, 3998, 3999, 4001, 4002, 4003, 4004, 4009, 4011, 4012, 4013, 4015, 4016, 4017, 4020, 4021, 4023, 4025, 4026, 4028, 4029, 4031, 4032, 4034, 4035, 4036, 4037, 4038, 4040, 4052, 4055, 4056, 4060, 4061, 4066, 4067, 4070, 4077, 4078, 4080, 4081, 4082, 4083, 4084, 4085, 4086, 4087, 4088, 4089, 4090, 4102, 4105, 4106, 4108, 4109, 4110, 4114, 4115, 4117, 4118, 4119, 4128, 4129, 4132, 4136, 4137, 4142, 4147, 4159, 4163, 4168, 4170, 4171, 4172, 4173, 4175, 4182, 4183, 4186, 4188, 4192, 4194, 4199, 4208, 4225, 4226, 4227, 4228, 4232, 4239, 4240, and 4258.

In some embodiments, the PEgRNA of the present disclosure provides a pegRNA sequence selected from any one of SEQ ID NOs: 4588, 4657, 4719, 4589, 4624, 4500, 4618, 4649, and 4533.

Another embodiment of the disclosure provides a prime editing guide RNA (PEgRNA) comprising: (a) a spacer that is complementary to a search target sequence on a first strand of an ATP7B gene, wherein the spacer comprises at its 3′ end nucleotides 5-20 of SEQ ID NO: 2293: (b) a gRNA core capable of binding to a Cas9 protein; (c) an extension arm comprising: (i) an editing template that comprises a region of complementarity to an editing target sequence on a second strand of the ATP7B gene, and (ii) a primer binding site that comprises at its 5′ end a sequence that is a reverse complement of nucleotides 15-17 of SEQ ID NO: 2293; wherein the first strand and second strand are complementary to each other and wherein the editing target sequence on the second strand is complementary to a portion of the ATP7B gene comprising a c.3207C>A substitution.

Another embodiments of the disclosure provides a prime editing guide RNA (PEgRNA) comprising: (a) a spacer comprising at its 3′ end nucleotides 5-20 of SEQ ID NO: 2293; (b) a gRNA core capable of binding to a Cas9 protein, and (c) an extension arm comprising: (i) an editing template comprising at its 3′ end any one of SEQ ID NOs: 2305-2422, and (ii) a primer binding site (PBS) sequence comprising at its 5′ end any one of SEQ ID NOs: 2294-2304.

In some embodiments, the spacer of the PEgRNA is from 15 to 22 nucleotides in length. In some embodiments, the spacer of the PEgRNA comprises at its 3′ end nucleotides 4-20, 3-20, 2-20, or 1-20 of SEQ ID NO: 2293. In some embodiments, the spacer of the PEgRNA comprises at its 3′ end SEQ ID NO: 2293. In some embodiments, the spacer of the PEgRNA is 20 nucleotides in length. In some embodiments, the PEgRNA of the present disclosure comprises from 5′ to 3′, the spacer, the gRNA core, the RTT, and the PBS. In some embodiments, the spacer, the gRNA core, the RTT, and the PBS form a contiguous sequence in a single molecule.

In some embodiments, the PEgRNA of the present disclosure comprises a pegRNA sequence selected from any one of SEQ ID NOs: 2445, 2446, 2447, 2448, 2449, 2450, 2451, 2452, 2453, 2454, 2455, 2456, 2457, 2458, 2459, 2460, 2461, 2462, 2463, 2464, 2465, 2466, 2467, 2468, 2469, 2470, 2471, 2472, 2473, 2474, 2475, 2476, 2477, 2478, 2479, 2480, 2481, 2482, 2483, 2484, 2485, 2486, 2487, 2488, 2489, 2490, 2491, 2492, 2493, 2494, 2495, 2496, 2497, 2498, 2499, 2500, 2501, 2502, 2503, 2504, 2505, 2506, 2507, 2508, 2509, 2510, 2511, 2512, 2513, 2514, 2515, 2516, 2517, 2518, 2519, 2520, 2521, 2522, 2523, 2524, 2525, 2526, 2527, 2528, 2529, 2530, 2531, 2532, 2533, 2534, 2535, 2538, 2539, 2540, 2541, 2542, 2543, 2544, 2545, 2546, 2547, 2548, 2549, 2550, 2551, 2553, 2554, 2555, 2556, 2557, 2558, 2559, 2560, 2561, 2562, 2563, 2564, 2565, 2566, 2567, 2568, 2569, 2570, 2571, 2572, 2573, 2574, 2575, 2576, 2577, 2578, 2580, 2582, 2584, 2585, 2586, 2587, 2588, 2589, 2590, 2591, 2592, 2593, 2594, 2595, 2596, 2597, 2598, 2600, 2601, 2602, 2603, 2605, 2606, 2607, 2608, 2609, 2610, 2611, 2612, 2613, 2614, 2615, 2616, 2617, 2618, 2619, 2620, 2621, 2623, 2624, 2628, 2629, 2630, 2631, 2632, 2633, 2634, 2635, 2636, 2637, 2638, 2643, 2645, 2646, 2647, 2648, 2649, 2650, 2651, 2652, 2653, 2654, 2655, 2656, 2657, 2658, 2659, 2660, 2663, 2664, 2665, 2667, 2668, 2669, 2670, 2671, 2672, 2674, 2675, 2676, 2677, 2678, 2680, 2681, 2683, 2685, 2687, 2688, 2689, 2690, 2692, 2694, 2695, 2696, 2697, 2699, 2701, 2702, 2704, 2706, 2708, 2711, 2713, 2715, 2716, 2717, 2720, 2721, 2722, 2723, 2725, 2726, 2727, 2728, 2729, 2730, 2733, 2734, 2735, 2744, 2747, 2748, 2749, 2752, 2753, 2757, 2758, 2759, 2760, 2761, 2762, 2764, 2765, 2768, 2769, 2770, 2772, 2773, 2774, 2777, 2786, 2788, 2789, 2790, 2791, 2792, 2793, 2794, 2795, 2796, 2797, 2798, 2799, 2800, 2801, 2802, 2807, 2810, 2811, 2812, 2814, 2816, 2824, 2825, 2826, 2828, 2829, 2830, 2832, 2833, 2834, 2841, 2842, 2843, 2844, 2846, 2847, 2854, 2855, 2856, 2857, 2862, 2864, 2866, 2867, 2868, 2869, 2870, 2871, 2885, 2886, 2887, 2888, 2889, 2890, 2891, 2893, 2894, 2896, 2898, 2899, 2901, 2902, 2909, 2910, 2914, 2916, 2918, 2919, 2920, 2926, 2927, 2932, 2933, 2937, 2938, 2939, 2941, 2942, 2945, 2953, 2954, 2956, 2957, 2960, 2962, 2963, 2964, 2965, 2967, 2972, 2973, 2977, 2979, 2980, 2982, 2983, 2988, 2991, 2993, 2994, 2995, 2997, 3006, 3008, 3012, 3013, 3015, 3023, 3024, 3027, 3028, 3029, 3030, 3031, 3032, 3033, 3034, 3035, 3036, 3037, 3038, 3039, 3043, 3044, 3045, 3046, 3048, 3049, 3050, 3051, 3052, 3053, 3054, 3055, 3059, 3064, 3065, 3071, 3072, 3075, 3076, 3080, 3082, 3084, 3093, 3096, 3098, 3099, 3101, 3119, 3121, 3122, 3123, 3124, 3126, 3128, 3130, 3133, 3142, 3144, 3148, 3159, 3161, 3162, 3163, 3164, 3165, 3166, 3168, 3169, 3170, 3176, 3182, 3188, 3190, 3191, 3195, 3200, 3202, 3203, 3210, 3212, 3216, 3218, 3226, 3227, 3228, 3229, 3230, 3231, 3232, 3234, 3235, 3238, 3239, 3241, 3243, 3244, 3245, 3246, 3247, 3248, 3249, 3250, 3260, 3262, 3263, 3271, 3273, 3275, 3281, 3282, 3283, 3287, 3288, 3289, 3300, 3301, 3302, 3303, 3304, 3305, 3307, 3310, 3311, 3312, 3313, 3314, 3315, 3316, 3317, 3318, 3322, 3324, 3325, 3328, 3330, 3346, 3347, 3348, 3349, 3350, 3358, 3359, 3362, 3364, 3365, 3366, 3367, 3368, 3372, 3373, 3382, 3385, 3387, 3388, 3389, 3390, 3391, 3392, 3393, 3400, 3403, 3404, 3405, 3407, 3408, 3409, 3412, 3414, 3420, 3423, 3425, 3426, 3427, 3428, 3429, 3430, 3431, 3434, 3438, 3441, 3442, 3446, 3449, 3450, 3451, 3452, 3453, 3454, 3455, 3463, 3466, 3469, 3470, 3471, 3472, 3473, 3474, 3477, 3478, 3480, 3481, 3482, 3487, 3490, 3494, 3498, 3499, 3502, 3503, 3505, 3506, 3508, 3509, 3510, 3511, 3513, 3520, 3522, 3523, 3526, 3529, 3533, 3535, 3536, 3542, 3543, 3546, 3547, 3549, 3550, 3553, 3554, 3555, 3557, 3560, 3561, 3563, 3564, 3567, 3568, 3569, 3571, 3574, 3575, 3576, 3578, 3579, 3580, 3581, 3583, 3584, 3585, 3592, 3594, 3595, 3596, 3597, 3603, 3612, 3613, 3617, 3622, 3625, 3626, 3627, 3628, 3630, 3631, 3632, 3633, 3635, 3636, 3638, 3639, 3640, 3641, 3642, 3646, 3647, 3648, 3654, 3657, 3659, 3660, 3661, 3664, 3668, 3669, 3673, 3674, 3678, 3679, 3680, 3681, 3684, 3685, 3687, 3688, 3697, 3699, 3702, 3703, 3704, 3705, 3706, 3708, 3710, 3711, 3712, 3714, 3715, 3721, 3722, 3724, 3725, 3728, 3729, 3730, 3731, 3732, 3733, 3734, 3735, 3736, 3737, 3739, 3740, 3741, 3743, 3744, 3746, 3748, 3755, 3761, 3770, 3771, 3773, 3774, 3776, 3778, 3779, 3781, 3782, 3784, 3785, 3792, 3793, 3794, 3795, 3796, 3797, 3798, 3799, 3800, 3801, 3802, 3803, 3804, 3805, 3806, 3807, 3808, 3809, 3810, 3811, 3812, 3814, 3815, 3816, 3820, 3829, 3839, 3841, 3842, 3843, 3844, 3845, 3851, 3852, 3853, 3854, 3855, 3856, 3857, 3858, 3859, 3860, 3861, 3862, 3863, 3864, 3865, 3868, 3869, 3871, 3874, 3875, 3876, 3877, 3878, 3879, 3880, 3882, 3883, 3884, 3885, 3887, 3895, 3899, 3904, 3907, 3908, 3909, 3910, 3911, 3912, 3913, 3914, 3915, 3916, 3917, 3921, 3924, 3927, 3928, 3929, 3931, 3932, 3935, 3936, 3937, 3938, 3939, 3940, 3941, 3942, 3943, 3945, 3946, 3956, 3957, 3961, 3962, 3965, 3971, 3977, 3978, 3979, 3980, 3981, 3982, 3983, 3985, 3988, 3989, 3990, 3991, 3992, 3993, 3994, 3995, 3997, 3998, 3999, 4001, 4002, 4003, 4004, 4009, 4011, 4012, 4013, 4015, 4016, 4017, 4020, 4021, 4023, 4025, 4026, 4028, 4029, 4031, 4032, 4034, 4035, 4036, 4037, 4038, 4040, 4052, 4055, 4056, 4060, 4061, 4066, 4067, 4070, 4077, 4078, 4080, 4081, 4082, 4083, 4084, 4085, 4086, 4087, 4088, 4089, 4090, 4102, 4105, 4106, 4108, 4109, 4110, 4114, 4115, 4117, 4118, 4119, 4128, 4129, 4132, 4136, 4137, 4142, 4147, 4159, 4163, 4168, 4170, 4171, 4172, 4173, 4175, 4182, 4183, 4186, 4188, 4192, 4194, 4199, 4208, 4225, 4226, 4227, 4228, 4232, 4239, 4240, and 4258.

In some embodiments, the PEgRNA of the present disclosure comprises a pegRNA sequence selected from any one of SEQ ID NOs: 2557, 2988, 2993, and 2585.

Another embodiment of the disclosure provides a prime editing system comprising: (a) the prime editing guide RNA (PEgRNA) of the present disclosure, or a nucleic acid encoding the PEgRNA; and (b) a nick guide RNA (ngRNA) comprising at its 3′ end nucleotides 5-20 of any one of SEQ ID NOs: 41, 60, 61, 62, 63, 64, 65, 66, 69, 71, 2045, 2046, 2047, 2048, 2049, 2050, 2051, 2052, 2053, 2054, 2055, 2056, 2057, 2058, 2059, 2060, 2061, 2062, 2063, 2064, 2065, 2066, 2067, 2068, 2069, 2070, 2071, 2072, 2073, 2074, 2075, 2076, 2077, 2078, 2079, 2080, 2081, 2082, 2083, 2084, 2085, 2086, 2087, 2088, 2089, 2090, 2091, 2092, 2093, 2094, 2095, 2096, 2423, 2424, 2425, 2426, 2427, 2428, 2429, 2430, 2431, 2432, 2433, 2434, 2435, 2436, 2437, 2438, 2439, 2440, 2441, 2442, 2443, and 2444 and a gRNA core capable of binding to a Cas9 protein, or a nucleic acid encoding the ngRNA.

In some embodiments, the spacer of the ngRNA is from 15 to 22 nucleotides in length. In some embodiments, the spacer of the ngRNA comprises at its 3′ end nucleotides 4-20, 3-20, 2-20, or 1-20 of any one of SEQ ID NOs: 41, 60, 61, 62, 63, 64, 65, 66, 69, 71, 2045, 2046, 2047, 2048, 2049, 2050, 2051, 2052, 2053, 2054, 2055, 2056, 2057, 2058, 2059, 2060, 2061, 2062, 2063, 2064, 2065, 2066, 2067, 2068, 2069, 2070, 2071, 2072, 2073, 2074, 2075, 2076, 2077, 2078, 2079, 2080, 2081, 2082, 2083, 2084, 2085, 2086, 2087, 2088, 2089, 2090, 2091, 2092, 2093, 2094, 2095, 2096, 2423, 2424, 2425, 2426, 2427, 2428, 2429, 2430, 2431, 2432, 2433, 2434, 2435, 2436, 2437, 2438, 2439, 2440, 2441, 2442, 2443, and 2444. In some embodiments, the spacer of the ngRNA comprises at its 3′ end of any one of SEQ ID NOs: 41, 60, 61, 62, 63, 64, 65, 66, 69, 71, 2045, 2046, 2047, 2048, 2049, 2050, 2051, 2052, 2053, 2054, 2055, 2056, 2057, 2058, 2059, 2060, 2061, 2062, 2063, 2064, 2065, 2066, 2067, 2068, 2069, 2070, 2071, 2072, 2073, 2074, 2075, 2076, 2077, 2078, 2079, 2080, 2081, 2082, 2083, 2084, 2085, 2086, 2087, 2088, 2089, 2090, 2091, 2092, 2093, 2094, 2095, 2096, 2423, 2424, 2425, 2426, 2427, 2428, 2429, 2430, 2431, 2432, 2433, 2434, 2435, 2436, 2437, 2438, 2439, 2440, 2441, 2442, 2443, and 2444.

In some embodiments, the spacer of the ngRNA is 20 nucleotides in length. In some embodiments, the ngRNA comprises SEQ ID NO: 2257, 2258, 2259, 2260, 2261, 2262, 2263, 2264, 2265, 2266, 2267, 2268, 2269, 2270, 2271, 2272, 2273, 2274, 2275, 2276, 2277, 2278, 2279, 2280, 2281, 2282, 2283, 2284, 2285, 2286, 2287, 2288, 2289, 4410, 4411, 4412, 4413, 4414, 4415, 4416, 4417, 4418, 4419, 4420, 4421, or 4422. In some embodiments, the ngRNA comprises SEQ ID NO: 2268, 2264, 4414, 4412, or 2265. In some embodiments, the prime editing system of present disclosure, further comprises: (c) a prime editor comprising a Cas9 nickase having a nuclease inactivating mutation in the HNH domain, or a nucleic acid encoding the Cas9 nickase, and a reverse transcriptase, or a nucleic acid encoding the reverse transcriptase.

In some embodiments, the Cas9 nickase comprises an amino acid sequence having at least 80%, 85%, 90%, 95%, 96%, 97%, 98%, 99%, or 100% identity to SEQ ID NO: 5786. In some embodiments, the reverse transcriptase comprises an amino acid sequence having at least 80%, 85%, 90%, 95%, 96%, 97%, 98%, 99%, or 100% identity to SEQ ID NO: 5842. In some embodiments, the sequence identities are determined by Needleman-Wunsch alignment of two protein sequences with Gap Costs set to Existence: 11 Extension: 1 where percent identity is calculated by dividing the number of identities by the length of the alignment. In some embodiments, the prime editor is a fusion protein.

Another embodiments of the disclosure provides an LNP comprising the prime editing system of the present disclosure. In some embodiments, the PEgRNA, the nucleic acid encoding the Cas9 nickase, and the nucleic acid encoding the reverse transcriptase. In some embodiments, the nucleic acid encoding the Cas9 nickase and the nucleic acid encoding the reverse transcriptase are mRNA. In some embodiments, the nucleic acid encoding the Cas9 nickase and the nucleic acid encoding the reverse transcriptase are the same molecule. In some embodiments, the LNP of the present disclosure, further comprises the ngRNA.

Another embodiment of the disclosure provides a method of correcting for editing an ATP7B gene, the method comprising contacting the ATP7B gene with: (a) the PEgRNA of the present disclosure and a prime editor comprising a Cas9 nickase having a nuclease inactivating mutation in the HNH domain and a reverse transcriptase, (b) the prime editing system of the present disclosure, or (c) the LNP of the present disclosure.

In some embodiments, the ATP7B gene is in a cell. In some embodiments, the cell is a mammalian cell. In some embodiments, the cell is a human cell. In some embodiments, the cell is a primary cell. In some embodiments, the cell is a hepatocyte. In some embodiments, the cell is in a subject. In some embodiments, the subject is a human. In some embodiments, the cell is from a subject having Wilson's disease. In some embodiments, the method of the present disclosure, further comprises administering the cell to the subject after incorporation of the intended nucleotide edit.

Another embodiment of the disclosure provides a cell generated by the method of the present disclosure.

Another embodiment of the disclosure provides a population of cells generated by the method of the present disclosure.

Another embodiment of the disclosure provides a method for treating Wilson's disease in a subject in need thereof, the method comprising administering to the subject: (a) the PEgRNA of the present disclosure, (b) the prime editing system of the present disclosure, or (c) the LNP of the present disclosure.

In some embodiments, the method of the present disclosure, comprises administering to the subject the PEgRNA of the present disclosure and a prime editor comprising a Cas9 nickase having a nuclease inactivating mutation in the HNH domain and a reverse transcriptase or one or more nucleic acids encoding the prime editor or its components. In some embodiments, the prime editor is a fusion protein. Another embodiment of the disclosure provides a prime editing guide RNA (PEgRNA) comprising: (a) a spacer comprising at its 3′ end nucleotides 5-20 of a PEgRNA Spacer sequence selected from any one of Tables 6-32; (b) a gRNA core capable of binding to a Cas9 protein, and (c) an extension arm comprising: (i) an editing template comprising at its 3′ end an RTT sequence selected from the same Table as the PEgRNA Spacer sequence, and (ii) a primer binding site (PBS) comprising at its 5′ end a PBS sequence selected from the same Table as the PEgRNA Spacer sequence.

In some embodiments, the spacer of the PEgRNA is from 15 to 22 nucleotides in length. In some embodiments, the spacer of the PEgRNA comprises at its 3′ end nucleotides 4-20, 3-20, 2-20, or 1-20 of the PEgRNA Spacer sequence selected from any one of Tables 6-32. In some embodiments, the spacer of the PEgRNA comprises at its 3′ end the PEgRNA Spacer sequence selected from any one of Tables 6-32. In some embodiments, the spacer of the PEgRNA is 20 nucleotides in length. In some embodiments, the PEgRNA of the disclosure, comprises from 5′ to 3′, the spacer, the gRNA core, the editing template, and the PBS. In some embodiments, the spacer, the gRNA core, the editing template, and the PBS form a contiguous sequence in a single molecule. In some embodiments, the PEgRNA of the present disclosure, comprises a pegRNA sequence selected from the same Table as the PEgRNA Spacer sequence. Another embodiment of the disclosure provides a prime editing system comprising: (a) the prime editing guide RNA (PEgRNA) of the present disclosure, or a nucleic acid encoding the PEgRNA; and (b) a nick guide RNA (ngRNA) comprising a spacer comprising at its 3′ end nucleotides 5-20 of any ngRNA Spacer sequence selected from the same Table as the PEgRNA Spacer sequence and a gRNA core capable of binding to a Cas9 protein, or a nucleic acid encoding the ngRNA.

In some embodiments, the spacer of the ngRNA is from 15 to 22 nucleotides in length. In some embodiments, the spacer of the ngRNA comprises at its 3′ end nucleotides 4-20, 3-20, 2-20, or 1-20 of the ngRNA Spacer sequence selected from the same Table as the PEgRNA Spacer sequence. In some embodiments, the spacer of the ngRNA comprises at its 3′ end the ngRNA Spacer sequence selected from the same Table as the PEgRNA Spacer sequence. In some embodiments, the spacer of the ngRNA is 20 nucleotides in length. In some embodiments, the ngRNA comprises a ngRNA sequence selected from the same Table as the PEgRNA Spacer sequence. In some embodiments, the prime editing system of the present disclosure, further comprises: (c) a prime editor comprising a Cas9 nickase having a nuclease inactivating mutation in the HNH domain, or a nucleic acid encoding the Cas9 nickase, and a reverse transcriptase, or a nucleic acid encoding the reverse transcriptase.

In some embodiments, the Cas9 nickase comprises an amino acid sequence having at least 80%, 85%, 90%, 95%, 96%, 9%, 98% 99%, or 100% identity to SEQ ID NO: 5786. In some embodiments, the reverse transcriptase comprises an amino acid sequence having at least 80%, 85%, 90%, 95%, 96%, 97%, 98%, 99%, or 100% identity to SEQ ID NO: 5842. In some embodiments, the sequence identities are determined by Needleman-Wunsch alignment of two protein sequences with Gap Costs set to Existence: 11 Extension: 1 where percent identity is calculated by dividing the number of identities by the length of the alignment. In some embodiments, the prime editor is a fusion protein.

Another embodiment of the disclosure provides an LNP comprising the prime editing system of the present disclosure. In some embodiments, the LNP of the present disclosure, comprises the PEgRNA, the nucleic acid encoding the Cas9 nickase, and the nucleic acid encoding the reverse transcriptase. In some embodiments, the nucleic acid encoding the Cas9 nickase and the nucleic acid encoding the reverse transcriptase are mRNA. In some embodiments, the nucleic acid encoding the Cas9 nickase and the nucleic acid encoding the reverse transcriptase are the same molecule. In some embodiments, the LNP of the present disclosure, further comprises the ngRNA.

Another embodiment of the disclosure provides a method of correcting for editing an ATP7B gene, the method comprising contacting the ATP7B gene with: (A) the PEgRNA of the present disclosure and a prime editor comprising a Cas9 nickase having a nuclease inactivating mutation in the HNH domain and a reverse transcriptase, (B) the prime editing system of the present disclosure, or (C) the LNP of the present disclosure.

In some embodiments, the ATP7B gene is in a cell. In some embodiments, the cell is a mammalian cell. In some embodiments, the cell is a human cell. In some embodiments, the cell is a primary cell. In some embodiments, the cell is a hepatocyte. In some embodiments, the cell is in a subject. In some embodiments, the subject is a human. In some embodiments, the cell is from a subject having Wilson's disease. In some embodiments, the method of the present disclosure, further comprises administering the cell to the subject after incorporation of the intended nucleotide edit.

Another embodiment of the disclosure provides a cell generated by the method of the present disclosure.

Another embodiment of the disclosure provides a population of cells generated by the method of the present disclosure.

Another embodiment of the disclosure provides a method for treating Wilson's disease in a subject in need thereof, the method comprising administering to the subject: (a) the PEgRNA of the present disclosure, (B) the prime editing system of the present disclosure, or (C) the LNP of the present disclosure.

In some embodiments, the method, comprises administering to the subject the PEgRNA of the present disclosure and a prime editor comprising a Cas9 nickase having a nuclease inactivating mutation in the HNH domain and a reverse transcriptase or one or more nucleic acids encoding the prime editor or its components. In some embodiments, the prime editor is a fusion protein.

Another embodiment of the disclosure provides a prime editing guide RNA (PEgRNA) comprising: a spacer that is complementary to a search target sequence on a first strand of an ATP7B gene, an editing template that comprises a region of complementarity to an editing target sequence on a second strand of the ATP7B gene, and a gRNA core that associates with a prime editor comprising a DNA binding domain and a DNA polymerase domain, wherein the first strand and second strand are complementary to each other, and wherein the editing target sequence is in an exon selected from the group consisting of: exon 8, exon 13, exon 14, exon 15, and exon 17 of the ATP7B gene.

Another embodiment of the disclosure provides a prime editing guide RNA (PEgRNA) comprising: a spacer that that is complementary to a search target sequence on a first strand of an ATP7B gene, an editing template that comprises a region of complementarity to an editing target sequence on a second strand of the ATP7B gene, and a gRNA core that associates with a prime editor comprising a DNA binding domain and a DNA polymerase domain, wherein first strand and second strand are complementary to each other, and wherein if the editing target sequence is in exon 3 then the editing target sequence does not comprise a c.1288 duplication as compared to a wild type ATP7B gene.

Another embodiment of the disclosure provides a prime editing guide RNA (PEgRNA) comprising: a spacer that is complementary to a search target sequence on a first strand of an ATP7B gene, an editing template that comprises a region of complementarity to an editing target sequence on a second strand of the ATP7B gene, and a gRNA core that associates with a prime editor comprising a DNA binding domain and a DNA polymerase domain, wherein first strand and second strand are complementary to each other, and wherein the editing target sequence is between positions 51932669-51946368 and positions 51932370-52012130 of human chromosome 13.

Another embodiment of the disclosure provides a prime editing guide RNA (PEgRNA) comprising: a spacer that is complementary to a search target sequence on a first strand of a ATP7B gene, an editing template that comprises a region of complementarity to an editing target sequence on a second strand of the ATP7B gene, and a gRNA core that associates with a prime editor comprising a DNA binding domain and a DNA polymerase domain, wherein first strand and second strand are complementary to each other, wherein the editing target sequence comprises a mutation associated with Wilson's disease, and wherein the mutation does not encode the amino acid substitution p.Ser430fs.

In some embodiments, the PEgRNA comprises a primer binding site sequence (PBS) at least partially complementary to the spacer. In some embodiments, wherein the gRNA core is between the spacer and the editing template. In some embodiments, the editing template comprises an intended nucleotide edit compared to the ATP7B gene. In some embodiments, the PEgRNA guides the prime editor to incorporate the intended nucleotide edit into the ATP7B gene when contacted with the ATP7B gene. In some embodiments, the prime editor synthesizes a single stranded DNA encoded by the editing template, wherein the single stranded DNA replaces the editing target sequence and results in incorporation of the intended nucleotide edit into a region corresponding to the editing target in the ATP7B gene.

In some embodiments, the search target sequence is complementary to a protospacer sequence in the APT7B gene, and wherein the protospacer sequence is adjacent to a search target adjacent motif (PAM) in the ATP7B gene. In some embodiments, the PEgRNA results in incorporation of the intended nucleotide edit in the PAM when contacted with the ATP7B gene. In some embodiments, the PBS is about 2 to 20 base pairs in length. In some embodiments, the PBS is about 8 to 16 base pairs in length. In some embodiments, the editing template is about 4 to 30 base pairs in length. In some embodiments, the editing template is about 10 to 30 base pairs in length. In some embodiments, the PEgRNA results in incorporation of intended nucleotide edit about 0 to 27 base pairs downstream of the 5 end of the PAM when contacted with the ATP7B gene. In some embodiments, the intended nucleotide edit comprises a single nucleotide substitution compared to the region corresponding to the editing target in the ATP7B gene.

In some embodiments, the intended nucleotide edit comprise an insertion compared to the region corresponding to the editing target in the ATP7B gene. In some embodiments, the intended nucleotide edit comprises a deletion compared to the region corresponding to the editing target in the ATP7B gene. In some embodiments, the editing target sequence comprises a mutation associated with Wilson's disease. In some embodiments, the editing template comprises a wild type ATP7B gene sequence. In some embodiments, the PEgRNA results in correction of the mutation when contacted with the ATP7B gene. In some embodiments, the editing target sequence is between positions 51944045 and 51944245 of human chromosome 13.

In some embodiments, the intended nucleotide edit comprises an A>C nucleotide substitution at position 51944145 in human chromosome 13 as compared to the region corresponding to the editing target in the ATP7B gene. In some embodiments, the editing target sequence comprises a mutation that encodes an H1069Q amino acid substitution as compared to a wild type ATP7B protein as set forth in SEQ ID NO:5861. In some embodiments, the spacer comprises a sequence selected from the group consisting of SEQ ID Nos. 1, 182, 294, 483, 682, 1505, 2023, 2293, 4425, 5206, 5228, 5248, 5282, 5313, 5340, 5369, 5406, 5423, 5446, 5473, 5503, 5537, 5555, 5638, and 5706. In some embodiments, the editing template comprises a sequence selected from the group consisting of SEQ ID Nos.:13-17, 194-198, 306-336, 495-528, 694-735, 1517-1546, 2035-2044, 2305-2422, 4437-4492, 5218, 5240-5247, 5260-5279, 5294-5302, 5325-5338, 5352-5368, 5381-5401, 5418-5422, 5435-5445, 5458-5472, 5485-5502, 5515-5535, 5549-5554, 5567-5590, 5650-5668, and 5718-5738.

In some embodiments, the PBS comprises a sequence selected from the group consisting of SEQ ID Nos. 2-12, 183-193, 295-305, 484-494, 683-693, 1506-1516, 2024-2034, 2294-2304, 4426-4436, 5207-5217, 5229-5239, 5249-5259, 5283-5293, 5314-5324, 5341-5351, 5370-5380, 5407-5417, 5424-5434, 5447-5457, 5474-5484, 5504-5514, 5538-5548, 5556-5566, 5639-5649, and 5707-5717.

In some embodiments, the spacer comprises a sequence selected from the group consisting of SEQ ID Nos. 1, 182, 294, 483, 682, 1505, 2023, 2293, 4425, 5206, 5228, 5248, 5282, 5313, 5340, 5369, 5406, 5423, 5446, 5473, 5503, 5537, 5555, 5638, and 5706.

In some embodiments, the editing template comprises a sequence selected from the group consisting of SEQ ID Nos. 13-17, 194-198, 306-336, 495-528, 694-735, 1517-1546, 2035-2044, 2305-2422, 4437-4492, 5218, 5240-5247, 5260-5279, 5294-5302, 5325-5338, 5352-5368, 5381-5401, 5418-5422, 5435-5445, 5458-5472, 5485-5502, 5515-5535, 5549-5554, 5567-5590, 5650-5668, and 5718-5738.

In some embodiments, the PEgRNA comprises a PBS selected from the group consisting of SEQ ID Nos. 2-12, 183-193, 295-305, 484-494, 683-693, 1506-1516, 2024-2034, 2294-2304, 4426-4436, 5207-5217, 5229-5239, 5249-5259, 5283-5293, 5314-5324, 5341-5351, 5370-5380, 5407-5417, 5424-5434, 5447-5457, 5474-5484, 5504-5514, 5538-5548, 5556-5566, 5639-5649, and 5707-5717.

Another embodiment of the disclosure provides a PEgRNA comprising a sequence selected from the group consisting of SEQ ID Nos. 73-152, 210-289, 338-482, 530-680, 741-1500, 1547-2022, 2097-2256, 2445-4409, 4493-5205, 5591-5637, 5669-5705, and 5739-5779.

Another embodiment of the disclosure provides a PEgRNA system comprising the PEgRNA of the present disclosure and further comprising a nick guide RNA (ngRNA), wherein the ngRNA comprises an ng spacer that is complementary to a second search target sequence in the ATP7B gene.

In some embodiments, the second search target sequence is on the second strand of the ATP7B gene. In some embodiments, the ng spacer comprises a sequence selected from the group consisting of SEQ ID Nos. 18-72, 199-209, 337, 529, 736-740, 2045-2096, 2423-2444, 5219-5227, 5280-5281, 5303-5312, 5339, 5402-5405, and 5536. In some embodiments, the ng spacer comprises a sequence selected form SEQ ID Nos. 2052, 2053, 2059, 2438, and 2441. Another embodiment of the disclosure provides a PEgRNA system comprising a PEgRNA selected from the group consisting of SEQ ID Nos. 73-152, 210-289, 338-482, 530-680, 741-1500, 1547-2022, 2097-2256, 2445-4409, 4493-5205, 5591-5637, 5669-5705, or 5739-5779 and a ngRNA selected from the group consisting of SEQ ID Nos. 2290, 2291, 2292, 4423, and 4424.

Another embodiment of the disclosure provides a PEgRNA system comprising a PEgRNA selected from the group consisting of SEQ ID Nos. 2739, 2785, 3276, 3277, 4536, 4613, 4695, 4721, 4741, 4743, 4762, 4788, and 4824 and a ngRNA selected from the group consisting of SEQ ID Nos. 2290, 2291, 2292, 4423, and 4424.

Another embodiment of the disclosure provides a prime editing complex comprising: (i) the PEgRNA of the present disclosure or the PEgRNA system of the present disclosure; and (ii) a prime editor comprising a DNA binding domain and a DNA polymerase domain.

In some embodiments, the DNA binding domain is a CRISPR associated (Cas) protein domain. In some embodiments, the Cas protein domain has nickase activity. In some embodiments, the Cas protein domain is a Cas9. In some embodiments, the Cas9 comprises a mutation in an HNH domain. In some embodiments, the Cas9 comprises a H840A mutation in the HNH domain. In some embodiments, the Cas protein domain is a Cas12b. In some embodiments, the Cas protein domain is a Cas12a, Cas12b, Cas12c, Cas12d, Cas12e, Cas14a, Cas14b, Cas14c, Cas14d, Cas14e, Cas14f, Cas14g, Cas14h, Cas14u, or a Casφ. In some embodiments, the DNA polymerase domain is a reverse transcriptase. In some embodiments, the reverse transcriptase is a retrovirus reverse transcriptase. In some embodiments, the reverse transcriptase is a Moloney murine leukemia virus (M-MLV) reverse transcriptase.

In some embodiments, the DNA polymerase and the programmable DNA binding domain are fused or linked to form a fusion protein. In some embodiments, the fusion protein comprises the sequence of SEQ ID NO: 10740.

Another embodiment of the disclosure provides a lipid nanoparticle (LNP) or ribonucleoprotein (RNP) comprising the prime editing complex of the present disclosure, or a component thereof.

Another embodiment of the disclosure provides a polynucleotide encoding the PEgRNA of the present disclosure, the PEgRNA system of the present disclosure, or the fusion protein of the present disclosure.

In some embodiments, the polynucleotide is a mRNA. In some embodiments, the polynucleotide is operably linked to a regulatory element. In some embodiments, the regulatory element is an inducible regulatory element.

Another embodiment of the disclosure provides a vector comprising the polynucleotide of the present disclosure. In some embodiments, the vector is an AAV vector.

Another embodiment of the disclosure provides an isolated cell comprising the PEgRNA of the present disclosure, the PEgRNA system of the present disclosure, the prime editing complex of the present disclosure, the LNP or RNP of the present disclosure, the polynucleotide of the present disclosure, or the vector of the present disclosure.

In some embodiments, the cell is a human cell. In some embodiments, the cell is a hepatocyte.

Another embodiment of the disclosure provides a pharmaceutical composition comprising (i) the PEgRNA of the present disclosure, the PEgRNA system of the present disclosure, the prime editing complex of the present disclosure, the LNP or RNP of the present disclosure, the polynucleotide of the present disclosure, the vector of the present disclosure, or the cell of the present disclosure; and (ii) a pharmaceutically acceptable carrier.

Another embodiment of the disclosure provides a method for editing an ATP7B gene, the method comprising contacting the ATP7B gene with (i) the PEgRNA of the present disclosure or the PEgRNA system of the present disclosure and (ii) a prime editor comprising a DNA binding domain and a DNA polymerase domain, wherein the PEgRNA directs the prime editor to incorporate the intended nucleotide edit in the ATP7B gene, thereby editing the ATP7B gene.

Another embodiment of the disclosure provides a method for editing an ATP7B gene, the method comprising contacting the ATP7B gene with the prime editing complex of the present disclosure, wherein the PEgRNA directs the prime editor to incorporate the intended nucleotide edit in the ATP7B gene, thereby editing the ATP7B gene.

In some embodiments, the prime editor synthesizes a single stranded DNA encoded by the editing template, wherein the single stranded DNA replaces the editing target sequence and results in incorporation of the intended nucleotide edit into a region corresponding to the editing target in the ATP7B gene. In some embodiments, the ATP7B gene is in a cell. In some embodiments, the cell is a mammalian cell. In some embodiments, the cell is a human cell. In some embodiments, wherein the cell is a primary cell. In some embodiments, the cell is a hepatocyte. In some embodiments, the cell is in a subject. In some embodiments, the subject is a human. In some embodiments, the cell is from a subject having Wilson's disease. In some embodiments, the method further comprises administering the cell to the subject after incorporation of the intended nucleotide edit.

Another embodiment of the disclosure provides a cell generated by the method of the present disclosure.

Another embodiment of the disclosure provides a population of cells generated by the method of the present disclosure.

Another embodiment of the disclosure provides a method for treating Wilson's disease in a subject in need thereof, the method comprising administering to the subject (i) the PEgRNA of the present disclosure or the PEgRNA system of the present disclosure and (ii) a prime editor comprising a DNA binding domain and a DNA polymerase domain, wherein the PEgRNA directs the prime editor to incorporate the intended nucleotide edit in the ATP7B gene in the subject, thereby treating Wilson's disease in the subject.

Another embodiment of the disclosure provides a method for treating Wilson's disease in a subject in need thereof, the method comprising administering to the subject the prime editing complex of the present disclosure, the LNP or RNP of the present disclosure, or the pharmaceutical composition of the present disclosure, wherein the PEgRNA directs the prime editor to incorporate the intended nucleotide edit in the ATP7B gene in the subject, thereby treating Wilson's disease in the subject.

In some embodiments, the subject is a human. In some embodiment, the ATP7B gene in the subject comprises a mutation that encodes an H1069Q amino acid substitution as compared to a wild type ATP7B protein as set forth in SEQ ID NO:5861. In some embodiment, the ATP7B gene comprises a mutation that encodes an H1069Q amino acid substitution as compared to a wild type ATP7B protein as set forth in SEQ ID NO:5861.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 depicts a schematic of a prime editing guide RNA (PEgRNA) binding to a double stranded target DNA sequence.

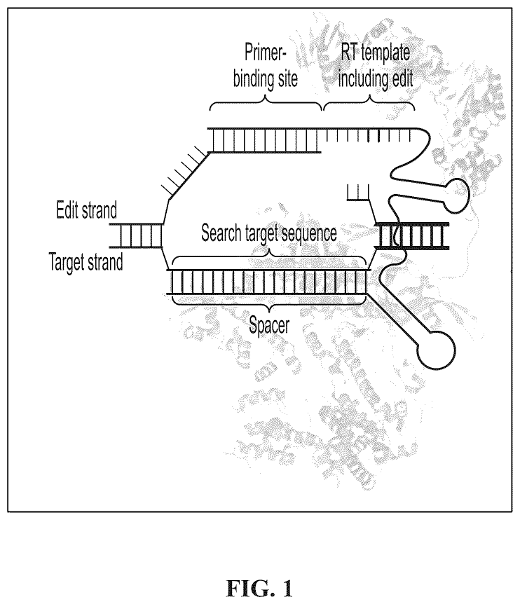

FIG. 2 depicts a PEgRNA architectural overview in an exemplary schematic of PEgRNA designed for a prime editor.

FIG. 3A depicts a 3′- to 5′ schematic (with the coding strand at the bottom) of an ATP7B H1069 locus with spacer sequences and an H1069Q mutation highlighted. FIG. 3B depicts a lentiviral screen design schematic.

FIG. 4 is a schematic showing the spacer and gRNA core part of an exemplary guide RNA, in two separate molecules. The rest of the PEgRNA structure is not shown.

### DETAILED DESCRIPTION OF THE INVENTION

Provided herein, in some embodiments, are compositions and methods to edit the target gene ATP7B with prime editing. In certain embodiments, provided herein are compositions and methods for correction of mutations in the copper-transporting ATPase 2 (ATP7B) gene associated with Wilson's Disease. Compositions provided herein can comprise prime editors (PEs) that may use engineered guide polynucleotides, e.g., prime editing guide RNAs (PEgRNAs), that can direct PEs to specific DNA targets and can encode DNA edits on the target gene ATP7B that serve a variety of functions, including direct correction of disease-causing mutations.

The following description and examples illustrate embodiments of the present disclosure in detail. It is to be understood that this disclosure is not limited to the particular embodiments described herein and as such can vary. Those of skill in the art will recognize that there are numerous variations and modifications of this disclosure, which are encompassed within its scope. Although various features of the present disclosure can be described in the context of a single embodiment, the features can also be provided separately or in any suitable combination. Conversely, although the present disclosure can be described herein in the context of separate embodiments for clarity, the present disclosure can also be implemented in a single embodiment.

### Definitions

Unless defined otherwise, all technical and scientific terms used herein have the same meaning as is commonly understood by one of ordinary skill in the art.

The terminology used herein is for the purpose of describing particular embodiments only and is not intended to be limiting. As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof as used herein mean “comprising”.

Unless otherwise specified, the words “comprising”, “comprise”, “comprises”, “having”, “have”, “has”, “including”, “includes”, “include”, “containing”, “contains” and “contain” are inclusive or open-ended and do not exclude additional, unrecited elements or method steps.

Reference to “some embodiments”, “an embodiment”, “one embodiment”, or “other embodiments” means that a particular feature or characteristic described in connection with the embodiments is included in at least one or more embodiments, but not necessarily all embodiments, of the present disclosure.

The term “about” or “approximately” means within an acceptable error range for the particular value as determined by one of ordinary skill in the art, which will depend in part on how the value is measured or determined, i.e., the limitations of the measurement system. For example, “about” can mean within 1 standard deviation, per the practice in the art. Alternatively, “about” can mean a range of up to 20%, up to 10%, up to 5%, or up to 1% of a given value. Alternatively, particularly with respect to biological systems or processes, the term can mean within an order of magnitude, preferably within 5-fold, and more preferably within 2-fold, of a value. Where particular values are described in the application and claims, unless otherwise stated, the term “about” meaning within an acceptable error range for the particular value should be assumed.

As used herein, a “cell” can generally refer to a biological cell. A cell can be the basic structural, functional and/or biological unit of a living organism. A cell can originate from any organism having one or more cells. Some non-limiting examples include: a prokaryotic cell, eukaryotic cell, a bacterial cell, an archaeal cell, a cell of a single-cell eukaryotic organism, a protozoa cell, a cell from a plant, an animal cell, a cell from an invertebrate animal (e.g. fruit fly, cnidarian, echinoderm, nematode, etc.), a cell from a vertebrate animal (e.g., fish, amphibian, reptile, bird, mammal), a cell from a mammal (e.g., a pig, a cow, a goat, a sheep, a rodent, a rat, a mouse, a non-human primate, a human, etc.), et cetera. Sometimes a cell may not originate from a natural organism (e.g., a cell can be synthetically made, sometimes termed an artificial cell).

In some embodiments, the cell is a human cell. A cell may be of or derived from different tissues, organs, and/or cell types. In some embodiments, the cell is a primary cell. As used herein, the term primary cell means a cell isolated from an organism, e.g., a mammal, which is grown in tissue culture (i.e., in vitro) for the first time before subdivision and transfer to a subculture. In some non-limiting examples, mammalian primary cells which can be transfected and further passaged include hepatocytes, fibroblasts, keratinocytes, epithelial cells (e.g., mammary epithelial cells, intestinal epithelial cells), endothelial cells, glial cells, neural cells, formed elements of the blood (e.g., lymphocytes, bone marrow cells), muscle cells and precursors of these somatic cell types. In some embodiments, the cell is a primary hepatocyte. In some embodiments, the cell is a primary human hepatocyte. In some embodiments, the cell is a primary human hepatocyte derived from an induced human pluripotent stem cell (iPSC). In some embodiments, the cell is a neuron. In some embodiments, the cell is a neuron from basal ganglia. In some embodiments, the cell is a neuron from basal ganglia of a subject.

In some embodiments, the cell comprises a prime editor or a prime editing composition. In some embodiments, the cell is from a human subject. In some embodiments, the human subject has a disease or condition associated with a mutation to be corrected by prime editing, for example, Wilsons's disease. In some embodiments, the cell is from a human subject, and comprises a prime editor or a prime editing composition for correction of the mutation. In some embodiments, the cell is from the human subject and the mutation has been edited or corrected by prime editing.

The term “substantially” as used herein may refer to a value approaching 100% of a given value. In some embodiments, the term may refer to an amount that may be at least about 70%, 80%, 90%, 91%, 92%, 93%, 94%, 95%, 96%, 97%, 98%, 99%, 99.9%, or 99.99% of a total amount. In some embodiments, the term may refer to an amount that may be about 100% of a total amount.

The terms “protein” and “polypeptide” can be used interchangeably to refer to a polymer of two or more amino acids joined by covalent bonds (e.g., an amide bond) that can adopt a three-dimensional conformation. In some embodiments, a protein or polypeptide comprises at least 10 amino acids, 15 amino acids, 20 amino acids, 30 amino acids or 50 amino acids joined by covalent bonds (e.g., amide bonds). In some embodiments, a protein comprises at least two amide bonds. In some embodiments, a protein comprises multiple amide bonds. In some embodiments, a protein comprises an enzyme, enzyme precursor proteins, regulatory protein, structural protein, receptor, nucleic acid binding protein, a biomarker, a member of a specific binding pair (e.g., a ligand or aptamer), or an antibody. In some embodiments, a protein may be a full-length protein (e.g., a fully processed protein having certain biological function). In some embodiments, a protein may be a variant or a fragment of a full-length protein. For example, in some embodiments, a Cas9 protein domain comprises an H840A amino acid substitution compared to a naturally occurring S. pyogenes Cas9 protein. A variant of a protein or enzyme, for example a variant reverse transcriptase, comprises a polypeptide having an amino acid sequence that is about 60% identical, about 70% identical, about 80% identical, about 90% identical, about 95% identical, about 96% identical, about 97% identical, about 98% identical, about 99% identical, about 99.5% identical, or about 99.9% identical to the amino acid sequence of a reference protein.

In some embodiments, a protein comprises one or more protein domains or subdomains. As used herein, the term “polypeptide domain”, “protein domain”, or “domain” when used in the context of a protein or polypeptide, refers to a polypeptide chain that has one or more biological functions, e.g., a catalytic function, a protein-protein binding function, or a protein-DNA function. In some embodiments, a protein comprises multiple protein domains. In some embodiments, a protein comprises multiple protein domains that are naturally occurring. In some embodiments, a protein comprises multiple protein domains from different naturally occurring proteins. For example, in some embodiments, a prime editor may be a fusion protein comprising a Cas9 protein domain of S. pyogenes and a reverse transcriptase protein domain of Moloney murine leukemia virus. A protein that comprises amino acid sequences from different origins or naturally occurring proteins may be referred to as a fusion, or chimeric protein.

In some embodiments, a protein comprises a functional variant or functional fragment of a full-length wild type protein. A “functional fragment” or “functional portion”, as used herein, refers to any portion of a reference protein (e.g., a wild type protein) that encompasses less than the entire amino acid sequence of the reference protein while retaining one or more of the functions, e.g., catalytic or binding functions. For example, a functional fragment of a reverse transcriptase may encompass less than the entire amino acid sequence of a wild type reverse transcriptase but retains the ability under at least one set of conditions to catalyze the polymerization of a polynucleotide. When the reference protein is a fusion of multiple functional domains, a functional fragment thereof may retain one or more of the functions of at least one of the functional domains. For example, a functional fragment of a Cas9 may encompass less than the entire amino acid sequence of a wild type Cas9 but retains its DNA binding ability and lacks its nuclease activity partially or completely.

A “functional variant” or “functional mutant”, as used herein, refers to any variant or mutant of a reference protein (e.g., a wild type protein) that encompasses one or more alterations to the amino acid sequence of the reference protein while retaining one or more of the functions, e.g., catalytic or binding functions. In some embodiments, the one or more alterations to the amino acid sequence comprises amino acid substitutions, insertions or deletions, or any combination thereof. In some embodiments, the one or more alterations to the amino acid sequence comprises amino acid substitutions. For example, a functional variant of a reverse transcriptase may comprise one or more amino acid substitutions compared to the amino acid sequence of a wild type reverse transcriptase but retains the ability under at least one set of conditions to catalyze the polymerization of a polynucleotide. When the reference protein is a fusion of multiple functional domains, a functional variant thereof may retain one or more of the functions of at least one of the functional domains. For example, in some embodiments, a functional fragment of a Cas9 may comprise one or more amino acid substitutions in a nuclease domain, e.g., an H840A amino acid substitution, compared to the amino acid sequence of a wild type Cas9, but retains the DNA binding ability and lacks the nuclease activity partially or completely.

The term “function” and its grammatical equivalents as used herein may refer to a capability of operating, having, or serving an intended purpose. Functional may comprise any percent from baseline to 100% of an intended purpose. For example, functional may comprise or comprise about 5%, 10%, 15%, 20%, 25%, 30%, 35%, 40%, 45%, 50%, 55%, 60%, 65%, 70%, 75%, 80%, 85%, 90%, 95%, or up to about 100% of an intended purpose. In some embodiments, the term functional may mean over or over about 100% of normal function, for example, 125%, 150%, 175%, 200%, 250%, 300%, 400%, 500%, 600%, 700% or up to about 1000% of an intended purpose.

In some embodiments, a protein or polypeptides includes naturally occurring amino acids (e.g., one of the twenty amino acids commonly found in peptides synthesized in nature, and known by the one letter abbreviations A, R, N, C, D, Q, E, G, H, I, L, K, M, F, P, S, T, W, Y and V). In some embodiments, a protein or polypeptides includes non-naturally occurring amino acids (e.g., amino acids which is not one of the twenty amino acids commonly found in peptides synthesized in nature, including synthetic amino acids, amino acid analogs, and amino acid mimetics). In some embodiments, a protein or polypeptide is modified.

In some embodiments, a protein comprises an isolated polypeptide. The term “isolated” means free or removed to varying degrees from components which normally accompany it as found in the natural state or environment. For example, a polypeptide naturally present in a living animal is not isolated, and the same polypeptide partially or completely separated from the coexisting materials of its natural state is isolated.

In some embodiments, a protein is present within a cell, a tissue, an organ, or a virus particle. In some embodiments, a protein is present within a cell or a part of a cell (e.g., a bacteria cell, a plant cell, or an animal cell). In some embodiments, the cell is in a tissue, in a subject, or in a cell culture. In some embodiments, the cell is a microorganism (e.g., a bacterium, fungus, protozoan, or virus). In some embodiments, a protein is present in a mixture of analytes (e.g., a lysate). In some embodiments, the protein is present in a lysate from a plurality of cells or from a lysate of a single cell.

The terms “homologous,” “homology,” or “percent homology” as used herein refer to the degree of sequence identity between an amino acid or polynucleotide sequence and a corresponding reference sequence. “Homology” can refer to polymeric sequences, e.g., polypeptide or DNA sequences that are similar. Homology can mean, for example, nucleic acid sequences with at least about: 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99%, or 100% identity. In other embodiments, a “homologous sequence” of nucleic acid sequences may exhibit 93%, 95% or 98% sequence identity to the reference nucleic acid sequence. For example, a “region of homology to a genomic region” can be a region of DNA that has a similar sequence to a given genomic region in the genome. A region of homology can be of any length that is sufficient to promote binding of a spacer or protospacer sequence to the genomic region. For example, the region of homology can comprise at least 5, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100, 200, 300, 400, 500, 600, 700, 800, 900, 1000, 1100, 1200, 1300, 1400, 1500, 1600, 1700, 1800, 1900, 2000, 2100, 2200, 2300, 2400, 2500, 2600, 2700, 2800, 2900, 3000, 3100 or more bases in length such that the region of homology has sufficient homology to undergo binding with the corresponding genomic region.

When a percentage of sequence homology or identity is specified, in the context of two nucleic acid sequences or two polypeptide sequences, the percentage of homology or identity generally refers to the alignment of two or more sequences across a portion of their length when compared and aligned for maximum correspondence. When a position in the compared sequence can be occupied by the same base or amino acid, then the molecules can be homologous at that position. Unless stated otherwise, sequence homology or identity is assessed over the specified length of the nucleic acid, polypeptide or portion thereof. In some embodiments, the homology or identity is assessed over a functional portion or specified portion of the length.

Alignment of sequences for assessment of sequence homology can be conducted by algorithms known in the art, such as the Basic Local Alignment Search Tool (BLAST) algorithm, which is described in Altschul et al, J. Mol. Biol. 215:403-410, 1990. A publicly available, internet interface, for performing BLAST analyses is accessible through the National Center for Biotechnology Information. Additional known algorithms include those published in: Smith & Waterman, “Comparison of Biosequences”, Adv. Appl. Math. 2:482, 1981; Needleman & Wunsch, “A general method applicable to the search for similarities in the amino acid sequence of two proteins” J. Mol. Biol. 48:443, 1970; Pearson & Lipman “Improved tools for biological sequence comparison”, Proc. Natl. Acad. Sci. USA 85:2444, 1988; or by automated implementation of these or similar algorithms. Global alignment programs can also be used to align similar sequences of roughly equal size. Examples of global alignment programs include NEEDLE (available at www.ebi.ac.uk/Tools/psa/emboss_needle/) which is part of the EMBOSS package (Rice P et al., Trends Genet., 2000; 16: 276-277), and the GGSEARCH program https://fasta.bioch.virginia.edu/fasta_www2/, which is part of the FASTA package (Pearson W and Lipman D, 1988, Proc. Natl. Acad. Sci. USA, 85: 2444-2448). Both of these programs are based on the Needleman-Wunsch algorithm which is used to find the optimum alignment (including gaps) of two sequences along their entire length. A detailed discussion of sequence analysis can also be found in Unit 19.3 of Ausubel et al (“Current Protocols in Molecular Biology” John Wiley & Sons Inc, 1994-1998, Chapter 15, 1998). In some embodiments, alignment between a query sequence and a reference sequence is performed with Needleman-Wunsch alignment with Gap Costs set to Existence: 11 Extension: 1 where percent identity is calculated by dividing the number of identities by the length of the alignment, as further described in Altschul et al. (“Gapped BLAST and PSI-BLAST: a new generation of protein database search programs”, Nucleic Acids Res. 25:3389-3402, 1997) and Altschul et al, (“Protein database searches using compositionally adjusted substitution matrices”, FEBS J. 272:5101-5109, 2005).

A skilled person understands that amino acid (or nucleotide) positions may be determined in homologous sequences based on alignment, for example, “H840” in a reference Cas9 sequence may correspond to H839, or another position in a Cas9 homolog.

The term “polynucleotide” or “nucleic acid molecule” can be any polymeric form of nucleotides, including DNA, RNA, a hybridization thereof, or RNA-DNA chimeric molecules. In some embodiments, a polynucleotide comprises cDNA, genomic DNA, mRNA, tRNA, rRNA, or microRNA. In some embodiments, a polynucleotide is double stranded, e.g., a double-stranded DNA in a gene. In some embodiments, a polynucleotide is single-stranded or substantially single-stranded, e.g., single-stranded DNA or an mRNA. In some embodiments, a polynucleotide is a cell-free nucleic acid molecule. In some embodiments, a polynucleotide circulates in blood. In some embodiments, a polynucleotide is a cellular nucleic acid molecule. In some embodiments, a polynucleotide is a cellular nucleic acid molecule in a cell circulating in blood.

Polynucleotides can have any three-dimensional structure. The following are nonlimiting examples of polynucleotides: a gene or gene fragment (for example, a probe, primer, EST or SAGE tag), an exon, an intron, intergenic DNA (including, without limitation, heterochromatic DNA), messenger RNA (mRNA), transfer RNA (tRNA), ribosomal RNA (rRNA), a ribozyme, cDNA, a recombinant polynucleotide, a branched polynucleotide, a plasmid, a vector, isolated DNA, isolated RNA, sgRNA, guide RNA, a nucleic acid probe, a primer, an snRNA, a long non-coding RNA, a snoRNA, a siRNA, a miRNA, a tRNA-derived small RNA (tsRNA), an antisense RNA, an shRNA, or a small rDNA-derived RNA (srRNA).

In some embodiments, a polynucleotide comprises deoxyribonucleotides, ribonucleotides or analogs thereof. In some embodiments, a polynucleotide comprises modified nucleotides, such as methylated nucleotides and nucleotide analogs. If present, modifications to the nucleotide structure can be imparted before or after assembly of the polynucleotide. The sequence of nucleotides can be interrupted by non-nucleotide components. A polynucleotide can be further modified after polymerization, such as by conjugation with a labeling component.

In some embodiments, a polynucleotide is composed of a specific sequence of four nucleotide bases: adenine (A); cytosine (C); guanine (G); thymine (T); and uracil (U) for thymine when the polynucleotide is RNA. In some embodiments, the polynucleotide may comprise one or more other nucleotide bases, such as inosine (I), which is read by the translation machinery as guanine (G).

In some embodiments, a polynucleotide may be modified. As used herein, the terms “modified” or “modification” refers to chemical modification with respect to the A, C, G, T and U nucleotides. In some embodiments, modifications may be on the nucleoside base and/or sugar portion of the nucleosides that comprise the polynucleotide. In some embodiments, the modification may be on the internucleoside linkage (e.g., phosphate backbone). In some embodiments, multiple modifications are included in the modified nucleic acid molecule. In some embodiments, a single modification is included in the modified nucleic acid molecule.

The term “complement”, “complementary”, or “complementarity” as used herein, refers to the ability of two polynucleotide molecules to base pair with each other. Complementary polynucleotides may base pair via hydrogen bonding, which may be Watson Crick, Hoogsteen or reversed Hoogsteen hydrogen bonding. For example, an adenine on one polynucleotide molecule will base pair to a thymine or an uracil on a second polynucleotide molecule and a cytosine on one polynucleotide molecule will base pair to a guanine on a second polynucleotide molecule. Two polynucleotide molecules are complementary to each other when a first polynucleotide molecule comprising a first nucleotide sequence can base pair with a second polynucleotide molecule comprising a second nucleotide sequence. For instance, the two DNA molecules 5′-ATGC-3′ and 5′-GCAT-3′ are complementary, and the complement of the DNA molecule 5′-ATGC-3′ is 5′-GCAT-3′. A percentage of complementarity indicates the percentage of nucleotides in a polynucleotide molecule which can base pair with a second polynucleotide molecule (e.g., 5, 6, 7, 8, 9, 10 out of 10 being 50%, 60%, 70%, 80%, 90%, and 100% complementary, respectively). “Perfectly complementary” means that all the contiguous nucleotides of a polynucleotide molecule will base pair with the same number of contiguous nucleotides in a second polynucleotide molecule. “Substantially complementary” as used herein refers to a degree of complementarity that can be 70%, 75%, 80%, 85%, 90%, 95%, 97%, 98%, or 99% over all or a portion of two polynucleotide molecules. In some embodiments, the portion of complementarity may be a region of 10, 15, 20, 25, 30, 35, 40, 45, 50, or more nucleotides. “Substantial complementary” can also refer to a 100% complementarity over a portion of two polynucleotide molecules. In some embodiments, the portion of complementarity between the two polynucleotide molecules is at least 60%, 65%, 70%, 75%, 80%, 85%, 90%, 95%, 97%, 98%, or 99% of the length of at least one of the two polynucleotide molecules or a functional or defined portion thereof.

As used herein, “expression” refers to the process by which polynucleotides are transcribed into mRNA and/or the process by which polynucleotides, e.g., the transcribed mRNA, translated into peptides, polypeptides, or proteins. If the polynucleotide is derived from genomic DNA, expression may include splicing of the mRNA in a eukaryotic cell. In some embodiments, expression of a polynucleotide, e.g., a gene or a DNA encoding a protein, is determined by the amount of the protein encoded by the gene after transcription and translation of the gene. In some embodiments, expression of a polynucleotide, e.g., a gene or a DNA encoding a protein, is determined by the amount of a functional form of the protein encoded by the gene after transcription and translation of the gene. In some embodiments, expression of a gene is determined by the amount of the mRNA, or transcript, that is encoded by the gene after transcription the gene. In some embodiments, expression of a polynucleotide, e.g., an mRNA, is determined by the amount of the protein encoded by the mRNA after translation of the mRNA. In some embodiments, expression of a polynucleotide, e.g., a mRNA or coding RNA, is determined by the amount of a functional form of the protein encoded by the polypeptide after translation of the polynucleotide.

The term “sequencing” as used herein, may comprise capillary sequencing, bisulfite-free sequencing, bisulfite sequencing, TET-assisted bisulfite (TAB) sequencing, ACE-sequencing, high-throughput sequencing, Maxam-Gilbert sequencing, massively parallel signature sequencing, Polony sequencing, 454 pyrosequencing, Sanger sequencing, Illumina sequencing, SOLiD sequencing, Ion Torrent semiconductor sequencing, DNA nanoball sequencing, Heliscope single molecule sequencing, single molecule real time (SMRT) sequencing, nanopore sequencing, shot gun sequencing, RNA sequencing, or any combination thereof.

The terms “equivalent” or “biological equivalent” are used interchangeably when referring to a particular molecule, or biological or cellular material, and means a molecule having minimal homology to another molecule while still maintaining a desired structure or functionality.

The term “encode” as it is applied to polynucleotides refers to a polynucleotide which is said to “encode” another polynucleotide, a polypeptide, or an amino acid if, in its native state or when manipulated by methods well known to those skilled in the art, it can be used as polynucleotide synthesis template, e.g., transcribed into an RNA, reverse transcribed into a DNA or cDNA, and/or translated to produce an amino acid, or a polypeptide or fragment thereof. In some embodiments, a polynucleotide comprising three contiguous nucleotides form a codon that encodes a specific amino acid. In some embodiments, a polynucleotide comprises one or more codons that encode a polypeptide. In some embodiments, a polynucleotide comprising one or more codons comprises a mutation in a codon compared to a wild-type reference polynucleotide. In some embodiments, the mutation in the codon encodes an amino acid substitution in a polypeptide encoded by the polynucleotide as compared to a wild-type reference polypeptide.

The term “mutation” as used herein refers to a change and/or alteration in an amino acid sequence of a protein or nucleic acid sequence of a polynucleotide. Such changes and/or alterations may comprise the substitution, insertion, deletion and/or truncation of one or more amino acids, in the case of an amino acid sequence, and/or nucleotides, in the case of nucleic acid sequence, compared to a reference amino acid or nucleic acid sequence. In some embodiments, the reference sequence is a wild-type sequence. In some embodiments, a mutation in a nucleic acid sequence of a polynucleotide encodes a mutation in the amino acid sequence of a polypeptide. In some embodiments, the mutation in the amino acid sequence of the polypeptide or the mutation in the nucleic acid sequence of the polynucleotide is a mutation associated with a disease state.

The term “subject” and its grammatical equivalents as used herein may refer to a human or a non-human. A subject may be a mammal. A human subject may be male or female. A human subject may be of any age. A subject may be a human embryo. A human subject may be a newborn, an infant, a child, an adolescent, or an adult. A human subject may be up to about 100 years of age. A human subject may be in need of treatment for a genetic disease or disorder.

The terms “treatment” or “treating” and their grammatical equivalents may refer to the medical management of a subject with an intent to cure, ameliorate, or ameliorate a symptom of, a disease, condition, or disorder. Treatment may include active treatment, that is, treatment directed specifically toward the improvement of a disease, condition, or disorder. Treatment may include causal treatment, that is, treatment directed toward removal of the cause of the associated disease, condition, or disorder. In addition, this treatment may include palliative treatment, that is, treatment designed for the relief of symptoms rather than the curing of the disease, condition, or disorder. Treatment may include supportive treatment, that is, treatment employed to supplement another specific therapy directed toward the improvement of the disease, condition, or disorder. In some embodiments, a condition may be pathological. In some embodiments, a treatment may not completely cure or prevent a disease, condition, or disorder. In some embodiments, a treatment ameliorates, but does not completely cure or prevent a disease, condition, or disorder. In some embodiments, a subject may be treated for 12 hours, 24 hours, 2 days, 3 days, 4 days, 5 days, 6 days, 7 days, 2 weeks, 3 weeks, 4 weeks, 2 months, 3 months, 4 months, 5 months, 6 months, 1 year, 2 years, 3 years, 4 years, 5 years, 6 years, indefinitely, or life of the subject.

The term “ameliorate” and its grammatical equivalents means to decrease, suppress, attenuate, diminish, arrest, or stabilize the development or progression of a disease.

The terms “prevent” or “preventing” means delaying, forestalling, or avoiding the onset or development of a disease, condition, or disorder for a period of time. Prevent also means reducing risk of developing a disease, disorder, or condition. Prevention includes minimizing or partially or completely inhibiting the development of a disease, condition, or disorder. In some embodiments, a composition, e.g. a pharmaceutical composition, prevents a disorder by delaying the onset of the disorder for 12 hours, 24 hours, 2 days, 3 days, 4 days, 5 days, 6 days, 7 days, 2 weeks, 3 weeks, 4 weeks, 2 months, 3 months, 4 months, 5 months, 6 months, 1 year, 2 years, 3 years, 4 years, 5 years, 6 years, indefinitely, or life of a subject.

The term “effective amount” or “therapeutically effective amount” may refer to a quantity of a composition, for example a composition comprising a construct, that can be sufficient to result in a desired activity upon introduction into a subject as disclosed herein. An effective amount of the prime editing compositions can be provided to the target gene or cell, whether the cell is ex vivo or in vivo. An effective amount can be the amount to induce, for example, at least about a 2-fold change (increase or decrease) or more in the amount of target nucleic acid modulation (e.g., expression of ATP7B gene to produce functional ATP7B protein) observed relative to a negative control. An effective amount or dose can induce, for example, about 2-fold increase, about 3-fold increase, about 4-fold increase, about 5-fold increase, about 6-fold increase, about 7-fold increase, about 8-fold increase, about 9-fold increase, about 10-fold increase, about 25-fold increase, about 50-fold increase, about 100-fold increase, about 200-fold increase, about 500-fold increase, about 700-fold increase, about 1000-fold increase, about 5000-fold increase, or about 10,000-fold increase in target gene modulation (e.g., expression of a target ATP7B gene to produce functional ATP7B protein). The amount of target gene modulation may be measured by any suitable method known in the art. In some embodiments, the “effective amount” or “therapeutically effective amount” is the amount of a composition that is required to ameliorate the symptoms of a disease relative to an untreated patient. In some embodiments, an effective amount is the amount of a composition sufficient to introduce an alteration in a gene of interest in a cell (e.g., a cell in vitro or in vivo).

As used herein, the terms “Wilson's disease,” “Wilsons disease,” and “Wilson disease” are used interchangeably. Wilson's disease is a monogenic autosomal-recessive disorder caused by pathogenic variants in ATP7B that decrease ATP7B function in hepatocytes and reduce excretion of excess copper into bile, leading to systemic copper buildup, hepatic and neural toxicity, and early demise. In some embodiments, mutations in the ATP7B gene are associated with diseases including Wilson's disease. The ATP7B gene codes for a copper transporter expressed in hepatic and neural tissues. The gene product is synthesized in the endoplasmic reticulum, then relocated to the trans Golgi network (TGN) within hepatocytes. ATP7B is most highly expressed in the liver, but is also found in the kidney, placenta, mammary glands, brain, and lung. Alternate names for ATP7B include: ATPase Copper Transporting Beta, Copper-Transporting ATPase, Copper Pump. ATPase, Cu++ Transporting. Beta Polypeptide, Wilson Disease-Associated Protein, PWD, WC1, WND, ATPase, Cu++ Transporting, Beta Polypeptide (Wilson Disease) 2. ATPase, Cu(2+)-Transporting, Beta Polypeptide, Copper-Transporting Protein ATP7B, Wilson Disease, EC 3.6.3.4, EC 7.2.2.8. EC 363, WD. In the human genome the ATP7B gene is located on 13q14.3 and contains 20 introns and 21 exons, for a total genomic length of 80 kb (chr13:51,930,436-52,012,130 (GRCh38/hg38)).

More than 600 pathogenic variants in ATP7B have been identified, with single-nucleotide missense and nonsense mutations being the most common, followed by insertions/deletions splice site mutations. A histidine-to-glutamate substitution at amino acid 1069 (p.H1069Q) (caused by c.3207C>A) in ATP7B maybe one of the most common cause of Wilson's disease, with a population allelic frequency of 10-40% (e.g., 30-70% among Caucasians. The p.H1069Q mutation occurs when histidine of the conserved SEHPL motif (SEQ ID NO: 5896) in the N-domain of ATP7B is replaced by glutamic acid, resulting in N-domain protein misfolding, abnormal phosphorylation in the P-domain, and decreased ATP binding affinity. This mutation may also lead to decreased heat stability and abnormal localization of the protein to the trans-Golgi network.

### Prime Editing

The term “prime editing” refers to programmable editing of a target DNA using a prime editor complexed with a PEgRNA to incorporate an intended nucleotide edit into the target DNA through target-primed DNA synthesis. A target gene of prime editing may comprise a double stranded DNA molecule having two complementary strands: a first strand that may be referred to as a “target strand” or a “non-edit strand”, and a second strand that may be referred to as a “non-target strand,” or an “edit strand.” In some embodiments, in a prime editing guide RNA (PEgRNA), a spacer sequence is complementary or substantially complementary to a specific sequence on the target strand, which may be referred to as a “search target sequence”. In some embodiments, the spacer sequence anneals with the target strand at the search target sequence. The target strand may also be referred to as the “non-Protospacer Adjacent Motif (non-PAM strand).” In some embodiments, the non-target strand may also be referred to as the “PAM strand”. In some embodiments, the PAM strand comprises a protospacer sequence and optionally a PAM sequence. A protospacer sequence refers to a specific sequence in the PAM strand of the target gene that is complementary to the search target sequence. In a PEgRNA, a spacer sequence may have a substantially identical sequence as the protospacer sequence on the edit strand of a target gene, except that the spacer sequence may comprise Uracil (U) and the protospacer sequence may comprise Thymine (T).

In some embodiments, the double stranded target DNA comprises a nick site on the PAM strand (or non-target strand). As used herein, a “nick site” refers to a specific position in between two nucleotides or two base pairs of the double stranded target DNA. In some embodiments, the position of a nick site is determined relative to the position of a specific PAM sequence. In some embodiments, the nick site is the particular position where a nick will occur when the double stranded target DNA is contacted with a nickase, for example, a Cas nickase, that recognizes a specific PAM sequence. In some embodiments, the nick site is upstream of a specific PAM sequence on the PAM strand of the double stranded target DNA. In some embodiments, the nick site is downstream of a specific PAM sequence on the PAM strand of the double stranded target DNA. In some embodiments, the nick site is upstream of a PAM sequence recognized by a Cas9 nickase, wherein the Cas9 nickase comprises a nuclease active RuvC domain and a nuclease inactive NHN domain. In some embodiments, the nick site is 3 nucleotides upstream of the PAM sequence, and the PAM sequence is recognized by a Streptococcus pyogenes Cas9 nickase, a P. lavamentivorans Cas9 nickase, a C. diphtheriae Cas9 nickase, a N. cinerea Cas9, a S. aureus Cas9, or a N. lari Cas9 nickase that comprises a nuclease active RuvC domain and a nuclease inactive NHN domain. In some embodiments, the nick site is 2 nucleotides upstream of the PAM sequence, and the PAM sequence is recognized by a S. thermophilus Cas9 nickase that comprises a nuclease active RuvC domain and a nuclease inactive NHN domain.

A “primer binding site” (also referred to as PBS or primer binding site sequence) is a single-stranded portion of the PEgRNA that comprises a region of complementarity to the PAM strand (i.e., the non-target strand or the edit strand). The PBS is complementary or substantially complementary to a sequence on the PAM strand of the double stranded target DNA that is immediately upstream of the nick site. In some embodiments, in the process of prime editing, the PEgRNA complexes with and directs a prime editor to bind the search target sequence on the target strand of the double stranded target DNA, and generates a nick at the nick site on the non-target strand of the double stranded target DNA. In some embodiments, the PBS is complementary to or substantially complementary to, and can anneal to, a free 3′ end on the non-target strand of the double stranded target DNA at the nick site. In some embodiments, the PBS annealed to the free 3′ end on the non-target strand can initiate target-primed DNA synthesis.

An “editing template” of a PEgRNA is a single-stranded portion of the PEgRNA that is 5′ of the PBS and comprises a region of complementarity to the PAM strand (i.e. the non-target strand or the edit strand), and comprises one or more intended nucleotide edits compared to the endogenous sequence of the double stranded target DNA. In some embodiments, the editing template and the PBS are immediately adjacent to each other. Accordingly, in some embodiments, a PEgRNA in prime editing comprises a single-stranded portion that comprises the PBS and the editing template immediately adjacent to each other. In some embodiments, the single stranded portion of the PEgRNA comprising both the PBS and the editing template is complementary or substantially complementary to an endogenous sequence on the PAM strand (i.e., the non-target strand or the edit strand) of the double stranded target DNA except for one or more non-complementary nucleotides at the intended nucleotide edit positions. As used herein, regardless of relative 5′-3′ positioning in other context, the relative positions as between the PBS and the editing template, and the relative positions as among elements of a PEgRNA, are determined by the 5′ to 3′ order of the PEgRNA as a single molecule regardless of the position of sequences in the double stranded target DNA that may have complementarity or identity to elements of the PEgRNA. In some embodiments, the editing template is complementary or substantially complementary to a sequence on the PAM strand that is immediately downstream of the nick site, except for one or more non-complementary nucleotides at the intended nucleotide edit positions. The endogenous, e.g., genomic, sequence that is complementary or substantially complementary to the editing template, except for the one or more non-complementary nucleotides at the position corresponding to the intended nucleotide edit, may be referred to as an “editing target sequence”. In some embodiments, the editing template has identity or substantial identity to a sequence on the target strand that is complementary to, or having the same position in the genome as, the editing target sequence, except for one or more insertions, deletions, or substitutions at the intended nucleotide edit positions. In some embodiments, the editing template encodes a single stranded DNA, wherein the single stranded DNA has identity or substantial identity to the editing target sequence except for one or more insertions, deletions, or substitutions at the positions of the one or more intended nucleotide edits.

In some embodiments, a PEgRNA complexes with and directs a prime editor to bind to the search target sequence of the target gene. In some embodiments, the bound prime editor generates a nick on the edit strand (PAM strand) of the target gene. In some embodiments, a primer binding site (PBS) of the PEgRNA anneals with a free 3′ end formed at the nick site, and the prime editor initiates DNA synthesis from the nick site, using the free 3′ end as a primer. Subsequently, a single-stranded DNA encoded by the editing template of the PEgRNA is synthesized. In some embodiments, the newly synthesized single-stranded DNA comprises one or more intended nucleotide edits compared to the endogenous target gene sequence. Accordingly, in some embodiments, the editing template of a PEgRNA is complementary to a sequence in the edit strand except for one or more mismatches at the intended nucleotide edit positions in the editing template. The endogenous, e.g., genomic, sequence that is partially complementary to the editing template may be referred to as an “editing target sequence”.

In some embodiments, the newly synthesized single-stranded DNA equilibrates with the editing target on the edit strand of the target gene for pairing with the target strand of the target gene. In some embodiments, the editing target sequence of the target gene is excised by a flap endonuclease (FEN), for example, FEN1. In some embodiments, the FEN is an endogenous FEN, for example, in a cell comprising the target gene. In some embodiments, the FEN is provided as part of the prime editor, either linked to other components of the prime editor or provided in trans. In some embodiments, the newly synthesized single stranded DNA, which comprises the intended nucleotide edit, replaces the endogenous single stranded editing target sequence on the edit strand of the target gene. In some embodiments, the newly synthesized single stranded DNA and the endogenous DNA on the target strand form a heteroduplex DNA structure at the region corresponding to the editing target sequence of the target gene. In some embodiments, the newly synthesized single-stranded DNA comprising the nucleotide edit is paired in the heteroduplex with the target strand of the target DNA that does not comprise the nucleotide edit, thereby creating a mismatch. In some embodiments, the mismatch is recognized by DNA repair machinery, e.g., an endogenous DNA repair machinery. In some embodiments, through DNA repair, the intended nucleotide edit is incorporated into the target gene.

### Prime Editor

The term “prime editor (PE)” refers to the polypeptide or polypeptide components involved in prime editing. In various embodiments, a prime editor includes a polypeptide domain having DNA binding activity and a polypeptide domain having DNA polymerase activity. In some embodiments, the prime editor further comprises a polypeptide domain having nuclease activity. In some embodiments, the polypeptide domain having DNA binding activity comprises a nuclease domain or nuclease activity. In some embodiments, the polypeptide domain having nuclease activity comprises a nickase, or a fully active nuclease. As used herein, the term “nickase” refers to a nuclease capable of cleaving only one strand of a double-stranded DNA target. In some embodiments, the prime editor comprises a polypeptide domain that is an inactive nuclease. In some embodiments, the polypeptide domain having programmable DNA binding activity comprises a nucleic acid guided DNA binding domain, for example, a CRISPR-Cas protein, for example, a Cas9 nickase, a Cpf1 nickase, or another CRISPR-Cas nuclease. In some embodiments, the polypeptide domain having DNA polymerase activity comprises a template-dependent DNA polymerase, for example, a DNA-dependent DNA polymerase or an RNA-dependent DNA polymerase. In some embodiments, the DNA polymerase is a reverse transcriptase. In some embodiments, the prime editor comprises additional polypeptides involved in prime editing, for example, a polypeptide domain having 5′ endonuclease activity, e.g., a 5′ endogenous DNA flap endonucleases (e.g., FEN1), for helping to drive the prime editing process towards the edited product formation. In some embodiments, the prime editor further comprises an RNA-protein recruitment polypeptide, for example, a MS2 coat protein.

A prime editor may be engineered. In some embodiments, the polypeptide components of a prime editor do not naturally occur in the same organism or cellular environment. In some embodiments, the polypeptide components of a prime editor may be of different origins or from different organisms. In some embodiments, a prime editor comprises a DNA binding domain and a DNA polymerase domain that are derived from different species. In some embodiments, a prime editor comprises a Cas polypeptide and a reverse transcriptase polypeptide that are derived from different species. For example, a prime editor may comprise a S. pyogenes Cas9 polypeptide and a Moloney murine leukemia virus (M-MLV) reverse transcriptase polypeptide.

In some embodiments, polypeptide domains of a prime editor may be fused or linked by a peptide linker to form a fusion protein. In other embodiments, a prime editor comprises one or more polypeptide domains provided in trans as separate proteins, which are capable of being associated to each other through non-peptide linkages or through aptamers or recruitment sequences. For example, a prime editor may comprise a DNA binding domain and a reverse transcriptase domain associated with each other by an RNA-protein recruitment aptamer, e.g. a MS2 aptamer, which may be linked to a PEgRNA. Prime editor polypeptide components may be encoded by one or more polynucleotides in whole or in part. In some embodiments, a single polynucleotide, construct, or vector encodes the prime editor fusion protein. In some embodiments, multiple polynucleotides, constructs, or vectors each encode a polypeptide domain or portion of a domain of a prime editor, or a portion of a prime editor fusion protein. For example, a prime editor fusion protein may comprise an N-terminal portion fused to an intein-N and a C-terminal portion fused to an intein-C, each of which is individually encoded by an AAV vector. In some embodiments, a prime editor comprises a nucleotide polymerase domain, e.g. a DNA polymerase domain. The DNA polymerase domain may be a wild-type DNA polymerase domain, a full-length DNA polymerase protein domain, or may be a functional mutant, a functional variant, or a functional fragment thereof. In some embodiments, the polymerase domain is a template dependent polymerase domain. For example, the DNA polymerase may rely on a template polynucleotide strand, e.g., the editing template sequence, for new strand DNA synthesis. In some embodiments, the prime editor comprises a DNA-dependent DNA polymerase. For example, a prime editor having a DNA-dependent DNA polymerase can synthesize a new single stranded DNA using a PEgRNA editing template that comprises a DNA sequence as a template. In such cases, the PEgRNA is a chimeric or hybrid PEgRNA, and comprising an extension arm comprising a DNA strand. The chimeric or hybrid PEgRNA may comprise an RNA portion (including the spacer and the gRNA core) and a DNA portion (the extension arm comprising the editing template that includes a strand of DNA). In some embodiments, the DNA polymerase is a bacteriophage polymerase, for example, a T4, T7, or phi29 DNA polymerase. In some embodiments, the DNA polymerase is an archaeal polymerase, for example, pol I type archaeal polymerase or a pol II type archaeal polymerase. In some embodiments, the DNA polymerase comprises a thermostable archaeal DNA polymerase. In some embodiments, the DNA polymerase comprises a eubacterial DNA polymerase, for example, Pol I, Pol II, or Pol III polymerase. In some embodiments, the DNA polymerase is a Pol I family DNA polymerase. In some embodiments, the DNA polymerase is a E. coli Pol I DNA polymerase. In some embodiments, the DNA polymerase is a Pol II family DNA polymerase. In some embodiments, the DNA polymerase is a Pyrococcusfuriosus (Pfu) Pol II DNA polymerase. In some embodiments, the DNA Polymerase is a Pol IV family DNA polymerase. In some embodiments, the DNA polymerase is a E. coli Pol IV DNA polymerase. In some embodiments, the DNA polymerase comprises a eukaryotic DNA polymerase. In some embodiments, the DNA polymerase is a Pol-beta DNA polymerase, a Pol-lamda DNA polymerase, a Pol-sigma DNA polymerase, or a Pol-mu DNA polymerase. In some embodiments, the DNA polymerase is a Pol-alpha DNA polymerase. In some embodiments, the DNA polymerase is a POLA1 DNA polymerase. In some embodiments, the DNA polymerase is a POLA2 DNA polymerase. In some embodiments, the DNA polymerase is a Pol-delta DNA polymerase. In some embodiments, the DNA polymerase is a POLD1 DNA polymerase. In some embodiments, the DNA polymerase is a POLD2 DNA polymerase. In some embodiments, the DNA polymerase is a human POLD1 DNA polymerase. In some embodiments, the DNA polymerase is a human POLD2 DNA polymerase. In some embodiments, the DNA polymerase is a POLD3 DNA polymerase. In some embodiments, the DNA polymerase is a POLD4 DNA polymerase. In some embodiments, the DNA polymerase is a Pol-epsilon DNA polymerase. In some embodiments, the DNA polymerase is a POLE1 DNA polymerase. In some embodiments, the DNA polymerase is a POLE2 DNA polymerase. In some embodiments, the DNA polymerase is a POLE3 DNA polymerase. In some embodiments, the DNA polymerase is a Pol-eta (POLH) DNA polymerase. In some embodiments, the DNA polymerase is a Pol-iota (POLI) DNA polymerase. In some embodiments, the DNA polymerase is a Pol-kappa (POLK) DNA polymerase. In some embodiments, the DNA polymerase is a Rev1 DNA polymerase. In some embodiments, the DNA polymerase is a human Rev1 DNA polymerase. In some embodiments, the DNA polymerase is a viral DNA-dependent DNA polymerase. In some embodiments, the DNA polymerase is a B family DNA polymerases. In some embodiments, the DNA polymerase is a herpes simplex virus (HSV) UL30 DNA polymerase. In some embodiments, the DNA polymerase is a cytomegalovirus (CMV) UL54 DNA polymerase.

In some embodiments, a prime editor comprises an RNA-dependent DNA polymerase domain, for example, a reverse transcriptase (RT). A RT or an RT domain may be a wild type RT domain, a full-length RT domain, or may be a functional mutant, a functional variant, or a functional fragment thereof. An RT or an RT domain of a prime editor may comprise a wild-type RT, or may be engineered or evolved to contain specific amino acid substitutions, truncations, or variants. An engineered RT may comprise sequences or amino acid changes different from a naturally occurring RT. In some embodiments, the engineered RT may have improved reverse transcription activity over a naturally occurring RT or RT domain. In some embodiments, the engineered RT may have improved features over a naturally occurring RT, for example, improved thermostability, reverse transcription efficiency, or target fidelity. In some embodiments, a prime editor comprising the engineered RT has improved prime editing efficiency over a prime editor having a reference naturally occurring RT.

In some embodiments, a prime editor comprises a virus RT, for example, a retrovirus RT. Non-limiting examples of virus RT include Moloney murine leukemia virus (M-MLV or MMLVRT); human T-cell leukemia virus type 1 (HTLV-1) RT; bovine leukemia virus (BLV) RT; Rous Sarcoma Virus (RSV) RT; human immunodeficiency virus (HIV) RT, M-MFV RT, Avian Sarcoma-Leukosis Virus (ASLV) RT, Rous Sarcoma Virus (RSV) RT, Avian Myeloblastosis Virus (AMV) RT, Avian Erythroblastosis Virus (AEV) Helper Virus MCAV RT, Avian Myelocytomatosis Virus MC29 Helper Virus MCAV RT, Avian Reticuloendotheliosis Virus (REV-T) Helper Virus REV-A RT, Avian Sarcoma Virus UR2 Helper Virus (UR2AV) RT, Avian Sarcoma Virus Y73 Helper Virus YAV RT, Rous Associated Virus (RAV) RT, and Myeloblastosis Associated Virus (MAV) RT, all of which may be suitably used in the methods and composition described herein.

In some embodiments, the prime editor comprises a wild-type M-MLV RT. An exemplary amino acid sequence of a wild-type M-MLV RT is provided in SEQ ID NO: 5780.

Exemplary wild type moloney murine leukemia virus reverse transcriptase:

(SEQ ID NO: 5780)
TLNIEDEHRLHETSKEPDVSLGSTWLSDFPQAWAETGGMGLAVRQAPLII
PLKATSTPVSIKQYPMSQEARLGIKPHIQRLLDQGILVPCQSPWNTPLLP
VKKPGTNDYRPVQDLREVNKRVEDIHPTVPNPYNLLSGLPPSHQWYTVLD
LKDAFFCLRLHPTSQPLFAFEWRDPEMGISGQLTWTRLPQGFKNSPTLFD
EALHRDLADFRIQHPDLILLQYVDDLLLAATSELDCQQGTRALLQTLGNL
GYRASAKKAQICQKQVKYLGYLLKEGQRWLTEARKETVMGQPTPKTPRQL
REFLGTAGFCRLWIPGFAEMAAPLYPLTKTGTLFNWGPDQQKAYQEIKQA
LLTAPALGLPDLTKPFELFVDEKQGYAKGVLTQKLGPWRRPVAYLSKKLD
PVAAGWPPCLRMVAAIAVLTKDAGKLTMGQPLVILAPHAVEALVKQPPDR
WLSNARMTHYQALLLDTDRVQFGPVVALNPATLLPLPEEGLQHNCLDILA
EAHGTRPDLTDQPLPDADHTWYTDGSSLLQEGQRKAGAAVTTETEVIWAK
ALPAGTSAQRAELIALTQALKMAEGKKLNVYTDSRYAFATAHIHGEIYRR
RGLLTSEGKEIKNKDEILALLKALFLPKRLSIIHCPGHQKGHSAEARGNR
MADQAARKAAITETPDTSTLLIENSSP.

In some embodiments, the prime editor comprises a reference M-MLV RT. An exemplary amino acid sequence of a reference M-MLV RT is provided in SEQ ID NO: 5781.

Exemplary reference moloney murine leukemia virus reverse transcriptase:

(SEQ ID NO: 5781)
TLNIEDEYRLHETSKEPDVSLGSTWLSDFPQAWAETGGMGLAVRQAPLII
PLKATSTPVSIKQYPMSQEARLGIKPHIQRLLDQGILVPCQSPWNTPLLP
VKKPGTNDYRPVQDLREVNKRVEDIHPTVPNPYNLLSGLPPSHQWYTVLD
LKDAFFCLRLHPTSQPLFAFEWRDPEMGISGQLTWTRLPQGFKNSPTLFD
EALHRDLADFRIQHPDLILLQYVDDLLLAATSELDCQQGTRALLQTLGNL
GYRASAKKAQICQKQVKYLGYLLKEGQRWLTEARKETVMGQPTPKTPRQL
REFLGTAGFCRLWIPGFAEMAAPLYPLTKTGTLFNWGPDQQKAYQEIKQA
LLTAPALGLPDLTKPFELFVDEKQGYAKGVLTQKLGPWRRPVAYLSKKLD
PVAAGWPPCLRMVAAIAVLTKDAGKLTMGQPLVILAPHAVEALVKQPPDR
WLSNARMTHYQALLLDTDRVQFGPVVALNPATLLPLPEEGLOHNCLDILA
EAHGTRPDLTDQPLPDADHTWYTDGSSLLQEGQRKAGAAVTTETEVIWAK
ALPAGTSAQRAELIALTQALKMAEGKKLNVYTDSRYAFATAHIHGEIYRR
RGLLTSEGKEIKNKDEILALLKALFLPKRLSIIHCPGHQKGHSAEARGNR
MADQAARKAAITETPDTSTLLIENSSP.

In some embodiments, the prime editor comprises a M-MLV RT comprising one or more of amino acid substitutions P51X, S67X, E69X, L139X, T197X, D200X, H204X, F209X, E302X, T306X, F309X, W313X, T330X, L345X, L435X, N454X, D524X, E562X, D583X, H594X, L603X, E607X, or D653X as compared to the reference M-MLV RT as set forth in SEQ ID NO: 5781, where X is any amino acid other than the original amino acid in the reference M-MLV RT. In some embodiments, the prime editor comprises a M-MLV RT comprising one or more of amino acid substitutions P51L, S67K, E69K, L139P, T197A, D200N, H204R, F209N, E302K, E302R, T306K, F309N, W313F, T330P, L345G, L435G, N454K, D524G, E562Q, D583N, H594Q, L603W, E607K, and D653N as compared to the reference M-MLV RT as set forth in SEQ ID NO: 5781. In some embodiments, the prime editor comprises a M-MLV RT comprising one or more amino acid substitutions D200N, T330P, L603W, T306K, and W313F as compared to the reference M-MLV RT as set forth in SEQ ID NO: 5781. In some embodiments, the prime editor comprises a M-MLV RT comprising amino acid substitutions D200N, T330P, L603W, T306K, and W313F as compared to the reference M-MLV RT as set forth in SEQ ID NO: 5781. In some embodiments, a prime editor comprising the D200N, T330P, L603W, T306K, and W313F as compared to a reference M-MLV RT may be referred to as a “PE2” prime editor, and the corresponding prime editing system a PE2 prime editing system.

In some embodiments, a prime editor comprises a eukaryotic RT, for example, a yeast, drosophila, rodent, or primate RT. In some embodiments, the prime editor comprises a Group II intron RT, for example, a. Geobacillus stearothermophilus Group II Intron (GsI-IIC) RT or a Eubacterium rectale group II intron (Eu.re.I2) RT. In some embodiments, the prime editor comprises a retron RT.

In some embodiments, the DNA-binding domain of a prime editor is a programmable DNA binding domain. In some embodiments, the prime editors provided herein comprise a DNA binding domain comprising an amino acid sequence that is at least 50%, at least 60%, at least 70%, at least 75%, at least 80%, 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to any one of the sequences set forth in SEQ ID NOs: 5783-5819. In some embodiments, the DNA binding domain comprises an amino acid sequence that has no more than 1, 2, 3, 4, 5, 6, 7, 8, 9, 10, 11, 12, 13, 14, 15, 16, 17, 18, 19, or 20 differences e.g., mutations e.g., deletions, substitutions and/or insertions compared to any one of the amino acid sequences set forth in SEQ ID NOs: 5783-5819. A programmable DNA binding domain refers to a protein domain that is designed to bind a specific nucleic acid sequence, e.g., a target DNA or a target RNA. In some embodiments, the DNA binding domain is a polynucleotide programmable DNA-binding domain that can associate with a guide polynucleotide (e.g., a PEgRNA) that guides the DNA-binding domain to a specific DNA sequence, e.g., a search target sequence in a target gene. In some embodiments, the DNA binding domain comprises a Clustered Regularly Interspaced Short Palindromic Repeats (CRISPR) Associated (Cas) protein. A Cas protein may comprise any Cas protein described herein or a functional fragment or functional variant thereof. In some embodiments, a DNA binding domain may also comprise a zinc-finger protein domain. In other cases, a DNA binding domain comprises a transcription activator-like effector domain (TALE). In some embodiments, the DNA binding domain comprises a DNA nuclease. For example, the DNA binding domain of a prime editor may comprise an RNA-guided DNA endonuclease, e.g., a Cas protein. In some embodiments, the DNA binding domain comprises a zinc finger nuclease (ZFN) or a transcription activator like effector domain nuclease (TALEN), where one or more zinc finger motifs or TALE motifs are associated with one or more nucleases, e.g., a Fok I nuclease domain.

In some embodiments, the DNA binding domain comprises a nuclease activity. In some embodiments, the DNA binding domain of a prime editor comprises an endonuclease domain having single strand DNA cleavage activity. For example, the endonuclease domain may comprise a FokI nuclease domain. In some embodiments, the DNA binding domain of a prime editor comprises a nuclease having full nuclease activity. In some embodiments, the DNA-binding domain of a prime editor comprises a nuclease having modified or reduced nuclease activity as compared to a wild type endonuclease domain. For example, the endonuclease domain may comprise one or more amino acid substitutions as compared to a wild type endonuclease domain. In some embodiments, the DNA binding domain of a prime editor has a nickase activity. In some embodiments, the DNA binding domain of a prime editor comprises a Cas protein domain that is a nickase. In some embodiments, compared to a wild type Cas protein, the Cas nickase comprises one or more amino acid substitutions in a nuclease domain that reduces or abolishes its double strand nuclease activity but retains DNA binding activity. In some embodiments, the Cas nickase comprises an amino acid substitution in a HNH domain. In some embodiments, the Cas nickase comprises an amino acid substitution in a RuvC domain.

In some embodiments, the DNA-binding domain comprises a CRISPR associated protein (Cas protein) domain. A Cas protein may be a Class 1 or a Class 2 Cas protein. A Cas protein can be a type I, type II, type III, type IV, type V Cas protein, or a type VI Cas protein. Non-limiting examples of Cas proteins include Cas1, Cas1B, Cas2, Cas3, Cas4, Cas5, Cas5d, Cas5t, Cas5h, Cas5a, Cas6, Cas7, Cas8, Cas8a, Cas8b, Cas8c, Cas9 (e.g., Csn1 or Csx12), Cas10, Cas10d, Cas12a/Cpf1, Cas12b/C2c1, Cas12c/C2c3, Cas12d/CasY, Cas12e/CasX, Cas12g, Cas12h, Cas12i, Csy1, Csy2, Csy3, Csy4, Cse1, Cse2, Cse3, Cse4, Cse5e, Csc1, Csc2, Csa5, Csn1, Csn2, Csm1, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx1S, Csx11, Csf1, Csf2, CsO, Csf4, Csd1, Csd2, Cst1, Cst2, Csh1, Csh2, Csa1, Csa2, Csa3, Csa4, Csa5, Type II Cas effector proteins, Type V Cas effector proteins, Type VI Cas effector proteins, CARF, DinG, Cpf1, Cas12b/C2c1, Cas12c/C2c3, Cas12b/C2c1, Cas12c/C2c3, SpCas9 (K855A), eSpCas9 (1.1), SpCas9-HF1, hyper accurate Cas9 variant (HypaCas9), Cas Φ, and homologues, modified or engineered variants, mutants, and/or functional fragments thereof. A Cas protein can be a chimeric Cas protein that is fused to other proteins or polypeptides. A Cas protein can be a chimera of various Cas proteins, for example, comprising domains of Cas proteins from different organisms. A Cas protein, e.g., Cas9, can be from any suitable organism. In some aspects, the organism is Streptococcus pyogenes (S. pyogenes). In some aspects, the organism is Staphylococcus aureus (S. aureus). In some aspects, the organism is Streptococcus thermophilus (S. thermophilus). In some embodiments, the organism is Staphylococcus lugdunensis. Non-limiting examples of suitable organism include Streptococcus pyogenes, Streptococcus thermophilus, Streptococcus sp., Staphylococcus aureus, Nocardiopsis dassonvillei, Streptomyces pristinae spiralis, Streptomyces viridochromo genes, Streptomyces viridochromogenes, Streptosporangium roseum, Streptosporangium roseum, AlicyclobacHlus acidocaldarius, Bacillus pseudomycoides, Bacillus selenitireducens, Exiguobacterium sibiricum, Lactobacillus delbrueckii, Lactobacillus salivarius, Microscilla marina, Burkholderiales bacterium, Polaromonas naphthalenivorans, Polaromonas sp., Crocosphaera watsonii, Cyanothece sp., Microcystis aeruginosa, Pseudomonas aeruginosa, Synechococcus sp., Acetohalobium arabaticum, Ammonifex degensii, Caldicelulosiruptor becscii, Candidatus Desulforudis, Clostridium botulinum, Clostridium difficile, Finegoldia magna, Natranaerobius thermophilus, Pelotomaculum thermopropionicum, Acidithiobacillus caldus, Acidithiobacillus ferrooxidans, Allochromatium vinosum, Marinobacter sp., Nitrosococcus halophilus, Nitrosococcus watsoni, Pseudoalteromonas haloplanktis, Ktedonobacter racemifer, Methanohalobium evestigatum, Anabaena variabilis, Nodularia spumigena, Nostoc sp., Arthrospira maxima, Arthrospira platensis, Arthrospira sp., Lyngbya sp., Microcoleus chthonoplastes, Oscillatoria sp., Petrotoga mobilis, Thermosipho africanus, Acaryochloris marina, Leptotrichia shahii, and Francisella novicida. In some embodiments, the organism is Streptococcus pyogenes (S. pyogenes). In some embodiments, the organism is Staphylococcus aureus (S. aureus). In some embodiments, the organism is Streptococcus thermophilus (S. thermophilus). In some embodiments, the organism is Staphylococcus lugdunensis (S. lugdunensis).

In some embodiments, a Cas protein can be derived from a variety of bacterial species including, but not limited to, Veillonella atypical, Fusobacterium nucleatum, Filifactor alocis, Solobacterium moorei, Coprococcus catus, Treponema denticola, Peptoniphilus duerdenii, Catenibacterium mitsuokai, Streptococcus mutans, Listeria innocua, Staphylococcus pseudintermedius, Acidaminococcus intestine, Olsenella uli, Oenococcus kitaharae, Bifidobacterium bifidum, Lactobacillus rhamnosus, Lactobacillus gasseri, Finegoldia magna, Mycoplasma mobile, Mycoplasma gallisepticum, Mycoplasma ovipneumoniae, Mycoplasma canis, Mycoplasma synoviae, Eubacterium rectale, Streptococcus thermophilus, Eubacterium dolichum, Lactobacillus coryniformis subsp. Torquens, Ilyobacter polytropus, Ruminococcus albus, Akkermansia muciniphila, Acidothermus cellulolyticus, Bifidobacterium longum, Bifidobacterium dentium, Corynebacterium diphtheria, Elusimicrobium minutum, Nitratifractor salsuginis, Sphaerochaeta globus, Fibrobacter succinogenes subsp. Succinogenes, Bacteroides fragilis, Capnocytophaga ochracea, Rhodopseudomonas palustris, Prevotella micans, Prevotella ruminicola, Flavobacterium columnare, Aminomonas paucivorans, Rhodospirillum rubrum, Candidatus Puniceispirillum marinum, Verminephrobacter eiseniae, Ralstonia syzygii, Dinoroseobacter shibae, Azospirillum, Nitrobacter hamburgensis, Bradyrhizobium, Wolinella succinogenes, Campylobacter jejuni subsp. Jejuni, Helicobacter mustelae, Bacillus cereus, Acidovorax ebreus, Clostridium perfringens, Parvibaculum lavamentivorans, Roseburia intestinalis, Neisseria meningitidis, Pasteurella multocida subsp. Multocida, Sutterella wadsworthensis, proteobacterium, Legionella pneumophila, Parasutterella excrementihominis, Wolinella succinogenes, and Francisella novicida.

In some embodiments, a Cas protein, e.g., Cas9, can be a wild type or a modified form of a Cas protein. In some embodiments, a Cas protein, e.g., Cas9, can be a nuclease active variant, nuclease inactive variant, a nickase, or a functional variant or functional fragment of a wild type Cas protein. In some embodiments, a Cas protein, e.g., Cas9, can comprise an amino acid change such as a deletion, insertion, substitution, fusion, chimera, or any combination thereof relative to a corresponding wild-type version of the Cas protein. In some embodiments, a Cas protein can be a polypeptide with at least about 50%, 60%, 70%, 80%, 90%, 91%, 92%, 93%, 94%, 95%, 96%, 97%, 98%, 99%, or 100% sequence identity or sequence similarity to a wild type exemplary Cas protein.

A Cas protein, e.g., Cas9, may comprise one or more domains. Non-limiting examples of Cas domains include, guide nucleic acid recognition and/or binding domain, nuclease domains (e.g., DNase or RNase domains, RuvC, HNH), DNA binding domain, RNA binding domain, helicase domains, protein-protein interaction domains, and dimerization domains. In various embodiments, a Cas protein comprises a guide nucleic acid recognition and/or binding domain can interact with a guide nucleic acid, and one or more nuclease domains that comprise catalytic activity for nucleic acid cleavage.

In some embodiments, a Cas protein, e.g., Cas9, comprises one or more nuclease domains. A Cas protein can comprise an amino acid sequence having at least about 50%, 60%, 70%, 80%, 90%, 91%, 92%, 93%, 94%, 95%, 96%, 97%, 98%, 99%, or 100% sequence identity to a nuclease domain (e.g., RuvC domain, HNH domain) of a wild-type Cas protein. In some embodiments, a Cas protein comprises a single nuclease domain. For example, a Cpf1 may comprise a RuvC domain but lacks HNH domain. In some embodiments, a Cas protein comprises two nuclease domains, e.g., a Cas9 protein can comprise an HNH nuclease domain and a RuvC nuclease domain.

In some embodiments, a prime editor comprises a Cas protein, e.g., Cas9, wherein all nuclease domains of the Cas protein are active. In some embodiments, a prime editor comprises a Cas protein having one or more inactive nuclease domains. One or a plurality of the nuclease domains (e.g., RuvC, HNH) of a Cas protein can be deleted or mutated so that they are no longer functional or comprise reduced nuclease activity. In some embodiments, a Cas protein, e.g., Cas9, comprising mutations in a nuclease domain has reduced (e.g. nickase) or abolished nuclease activity while maintaining its ability to target a nucleic acid locus at a search target sequence when complexed with a guide nucleic acid, e.g. a PEgRNA.

In some embodiments, a prime editor comprises a Cas nickase that can bind to the target gene in a sequence-specific manner and generate a single-strand break at a protospacer within double-stranded DNA in the target gene, but not a double-strand break. For example, the Cas nickase can cleave the edit strand or the non-edit strand of the target gene, but may not cleave both. In some embodiments, a prime editor comprises a Cas nickase comprising two nuclease domains (e.g., Cas9), with one of the two nuclease domains modified to lack catalytic activity or deleted. In some embodiments, the Cas nickase of a prime editor comprises a nuclease inactive RuvC domain and a nuclease active HNH domain. In some embodiments, the Cas nickase of a prime editor comprises a nuclease inactive HNH domain and a nuclease active RuvC domain. In some embodiments, a prime editor comprises a Cas9 nickase having an amino acid substitution in the RuvC domain that reduces or abolishes nuclease activity of the RuvC domain. In some embodiments, the Cas9 nickase comprises a D10X amino acid substitution compared to a wild type S. pyogenes Cas9, wherein X is any amino acid other than D. In some embodiments, a prime editor comprises a Cas9 nickase having an amino acid substitution in the HNH domain that reduces or abolishes nuclease activity of the HNH domain. In some embodiments, the Cas9 nickase comprises a H840X amino acid substitution compared to a wild type S. pyogenes Cas9, wherein X is any amino acid other than H.

In some embodiments, a prime editor comprises a Cas protein that can bind to the target gene in a sequence-specific manner but lacks or has abolished nuclease activity and may not cleave either strand of a double stranded DNA in a target gene. Abolished activity or lacking activity can refer to an enzymatic activity less than 1%, less than 2%, less than 3%, less than 4%, less than 5%, less than 6%, less than 7%, less than 8%, less than 9%, or less than 10% activity compared to a wild-type exemplary activity (e.g., wild-type Cas9 nuclease activity). In some embodiments, a Cas protein of a prime editor completely lacks nuclease activity. A nuclease, e.g., Cas9, that lacks nuclease activity may be referred to as nuclease inactive or “nuclease dead” (abbreviated by “d”). A nuclease dead Cas protein (e.g., dCas, dCas9) can bind to a target polynucleotide but may not cleave the target polynucleotide. In some embodiments, a dead Cas protein is a dead Cas9 protein. In some embodiments, a prime editor comprises a nuclease dead Cas protein wherein all of the nuclease domains (e.g., both RuvC and HNH nuclease domains in a Cas9 protein; RuvC nuclease domain in a Cpf1 protein) are mutated to lack catalytic activity, or are deleted.

A Cas protein can be modified. A Cas protein, e.g., Cas9, can be modified to increase or decrease nucleic acid binding affinity, nucleic acid binding specificity, and/or enzymatic activity. Cas proteins can also be modified to change any other activity or property of the protein, such as stability. For example, one or more nuclease domains of the Cas protein can be modified, deleted, or inactivated, or a Cas protein can be truncated to remove domains that are not essential for the function of the protein or to optimize (e.g., enhance or reduce) the activity of the Cas protein.

A Cas protein can be a fusion protein. For example, a Cas protein can be fused to a cleavage domain, an epigenetic modification domain, a transcriptional regulation domain, or a polymerase domain. A Cas protein can also be fused to a heterologous polypeptide providing increased or decreased stability. The fused domain or heterologous polypeptide can be located at the N-terminus, the C-terminus, or internally within the Cas protein.

In some embodiments, the Cas protein of a prime editor is a Class 2 Cas protein. In some embodiments, the Cas protein is a type II Cas protein. In some embodiments, the Cas protein is a Cas9 protein, a modified version of a Cas9 protein, a Cas9 protein homolog, mutant, variant, or a functional fragment thereof. As used herein, a Cas9, Cas9 protein, Cas9 polypeptide or a Cas9 nuclease refers to an RNA guided nuclease comprising one or more Cas9 nuclease domains and a Cas9 gRNA binding domain having the ability to bind a guide polynucleotide, e.g., a PEgRNA. A Cas9 protein may refer to a wild type Cas9 protein from any organism or a homolog, ortholog, or paralog from any organisms; any functional mutants or functional variants thereof; or any functional fragments or domains thereof. In some embodiments, a prime editor comprises a full-length Cas9 protein. In some embodiments, the Cas9 protein can generally comprises at least about 50%, 60%, 70%, 80%, 90%, 100% sequence identity to a wild type reference Cas9 protein (e.g., Cas9 from S. pyogenes). In some embodiments, the Cas9 comprises an amino acid change such as a deletion, insertion, substitution, fusion, chimera, or any combination thereof as compared to a wild type reference Cas9 protein.

In some embodiments, a Cas9 protein may comprise a Cas9 protein from Streptococcus pyogenes (Sp), Staphylococcus aureus (Sa), Streptococcus canis (Sc), Streptococcus thermophilus (St), Staphylococcus lugdunensis (Slu), Neisseria meningitidis (Nm), Campylobacter jejuni (Cj), Francisella novicida (Fn), or Treponema denticola (Td), or any Cas9 homolog or ortholog from an organism known in the art. In some embodiments, a Cas9 polypeptide is a SpCas9 polypeptide. In some embodiments, a Cas9 polypeptide is a SaCas9 polypeptide. In some embodiments, a Cas9 polypeptide is a ScCas9 polypeptide. In some embodiments, a Cas9 polypeptide is a StCas9 polypeptide. In some embodiments, a Cas9 polypeptide is a SluCas9 polypeptide. In some embodiments, a Cas9 polypeptide is a NmCas9 polypeptide. In some embodiments, a Cas9 polypeptide is a CjCas9 polypeptide. In some embodiments, a Cas9 polypeptide is a FnCas9 polypeptide. In some embodiments, a Cas9 polypeptide is a TdCas9 polypeptide. In some embodiments, a Cas9 polypeptide is a chimera comprising domains from two or more of the organisms described herein or those known in the art. In some embodiments, a Cas9 polypeptide is a Cas9 polypeptide from Streptococcus macacae. In some embodiments, a Cas9 polypeptide is a Cas9 polypeptide generated by replacing a PAM interaction domain of a SpCas9 with that of a Streptococcus macacae Cas9 (Spy-mac Cas9).

In some embodiments, a Cas9 protein comprises a Cas9 protein from Streptococcus pyogenes (Sp), e.g., as according to NC_002737.2:854751-858857 or the protein encoded by UniProt Q99ZW2, e.g., as according to SEQ ID NO: 5783. In some embodiments, the Cas9 protein is a SpCas9. In some embodiments, a SpCas9 can be a wild type SpCas9, a SpCas9 variant, or a nickase SpCas9. In some embodiments, the SpCas9 lacks the N-terminus methionine relative to a corresponding SpCas9 (e.g., wild type SpCas9, a SpCas9 variant or a nickase SpCas9). In some embodiments, a prime editor comprises a Cas9 protein, having an amino acid sequence as according to SEQ ID NO: 5783, not including the N-terminus methionine. In some embodiments, a wild type SpCas9 comprises an amino acid sequence set forth in SEQ ID NO: 5783 or SEQ ID NO: 5784. In some embodiments, a prime editor comprises a Cas9 protein, having an amino acid sequence as according to SEQ ID NO: 5783, not including the N-terminus methionine (e.g., as set forth in SEQ ID NO: 5784). In some embodiments, a prime editor comprises a Cas9 protein comprising one or more mutations (e.g., amino acid substitutions, insertions and/or deletions relative to a corresponding wild type Cas9 protein (e.g., wild type SpCas9). In some embodiments, the Cas9 protein comprising one or mutations relative to a wild type Cas9 protein comprises an amino acid sequence set forth in SEQ ID NO: 5785.

Exemplary Streptococcus pyogenes Cas9 (SpCas9) amino acid sequences useful in the prime editors disclosed herein are provided below in SEQ ID NOs: 5783-5789.

Exemplary wild type Streptococcus pyogenes Cas9 (SpCas9) amino acid sequence:

(SEQ ID NO: 5783)
MDKKYSIGLDIGTNSVGWAVITDEYKVPSKKFKVLGNTDRHSIKKNLIGA
LLFDSGETAEATRLKRTARRRYTRRKNRICYLQEIFSNEMAKVDDSFFHR
LEESFLVEEDKKHERHPIFGNIVDEVAYHEKYPTIYHLRKKLVDSTDKAD
LRLIYLALAHMIKFRGHFLIEGDLNPDNSDVDKLFIQLVQTYNQLFEENP
INASGVDAKAILSARLSKSRRLENLIAQLPGEKKNGLFGNLIALSLGLTP
NFKSNFDLAEDAKLQLSKDTYDDDLDNLLAQIGDQYADLFLAAKNLSDAI
LLSDILRVNTEITKAPLSASMIKRYDEHHQDLTLLKALVRQQLPEKYKEI
FFDQSKNGYAGYIDGGASQEEFYKFIKPILEKMDGTEELLVKLNREDLLR
KQRTFDNGSIPHQIHLGELHAILRRQEDFYPFLKDNREKIEKILTFRIPY
YVGPLARGNSRFAWMTRKSEETITPWNFEEVVDKGASAQSFIERMTNFDK
NLPNEKVLPKHSLLYEYFTVYNELTKVKYVTEGMRKPAFLSGEQKKAIVD
LLFKTNRKVTVKQLKEDYFKKIECFDSVEISGVEDRFNASLGTYHDLLKI
IKDKDFLDNEENEDILEDIVLTLTLFEDREMIEERLKTYAHLFDDKVMKQ
LKRRRYTGWGRLSRKLINGIRDKQSGKTILDFLKSDGFANRNFMQLIHDD
SLTFKEDIQKAQVSGQGDSLHEHIANLAGSPAIKKGILQTVKVVDELVKV
MGRHKPENIVIEMARENQTTQKGQKNSRERMKRIEEGIKELGSQILKEHP
VENTQLQNEKLYLYYLQNGRDMYVDQELDINRLSDYDVDHIVPQSFLKDD
SIDNKVLTRSDKNRGKSDNVPSEEVVKKMKNYWRQLLNAKLITQRKFDNL
TKAERGGLSELDKAGFIKRQLVETRQITKHVAQILDSRMNTKYDENDKLI
REVKVITLKSKLVSDFRKDFQFYKVREINNYHHAHDAYLNAVVGTALIKK
YPKLESEFVYGDYKVYDVRKMIAKSEQEIGKATAKYFFYSNIMNFFKTEI
TLANGEIRKRPLIETNGETGEIVWDKGRDFATVRKVLSMPQVNIVKKTEV
QTGGFSKESILPKRNSDKLIARKKDWDPKKYGGFDSPTVAYSVLVVAKVE
KGKSKKLKSVKELLGITIMERSSFEKNPIDFLEAKGYKEVKKDLIIKLPK
YSLFELENGRKRMLASAGELQKGNELALPSKYVNFLYLASHYEKLKGSPE
DNEQKQLFVEQHKHYLDEIIEQISEFSKRVILADANLDKVLSAYNKHRDK
PIREQAENIIHLFTLTNLGAPAAFKYFDTTIDRKRYTSTKEVLDATLIHQ
SITGLYETRIDLSQLGGD.

Exemplary wild type Streptococcus pyogenes Cas9 (SpCas9) amino acid sequence lacking the N-terminus methionine:

(SEQ ID NO: 5784)
DKKYSIGLDIGTNSVGWAVITDEYKVPSKKFKVLGNTDRHSIKKNLIGAL
LFDSGETAEATRLKRTARRRYTRRKNRICYLQEIFSNEMAKVDDSFFHRL
EESFLVEEDKKHERHPIFGNIVDEVAYHEKYPTIYHLRKKLVDSTDKADL
RLIYLALAHMIKFRGHFLIEGDLNPDNSDVDKLFIQLVQTYNQLFEENPI
NASGVDAKAILSARLSKSRRLENLIAQLPGEKKNGLFGNLIALSLGLTPN
FKSNFDLAEDAKLQLSKDTYDDDLDNLLAQIGDQYADLFLAAKNLSDAIL
LSDILRVNTEITKAPLSASMIKRYDEHHQDLTLLKALVRQQLPEKYKEIF
FDQSKNGYAGYIDGGASQEEFYKFIKPILEKMDGTEELLVKLNREDLLRK
QRTFDNGSIPHQIHLGELHAILRRQEDFYPFLKDNREKIEKILTFRIPYY
VGPLARGNSRFAWMTRKSEETITPWNFEEVVDKGASAQSFIERMTNFDKN
LPNEKVLPKHSLLYEYFTVYNELTKVKYVTEGMRKPAFLSGEQKKAIVDL
LFKTNRKVTVKQLKEDYFKKIECFDSVEISGVEDRFNASLGTYHDLLKII
KDKDFLDNEENEDILEDIVLTLTLFEDREMIEERLKTYAHLFDDKVMKQL
KRRRYTGWGRLSRKLINGIRDKQSGKTILDFLKSDGFANRNFMQLIHDDS
LTFKEDIQKAQVSGQGDSLHEHIANLAGSPAIKKGILQTVKVVDELVKVM
GRHKPENIVIEMARENQTTQKGQKNSRERMKRIEEGIKELGSQILKEHPV
ENTQLQNEKLYLYYLQNGRDMYVDQELDINRLSDYDVDHIVPQSFLKDDS
IDNKVLTRSDKNRGKSDNVPSEEVVKKMKNYWRQLLNAKLITQRKFDNLT
KAERGGLSELDKAGFIKRQLVETRQITKHVAQILDSRMNTKYDENDKLIR
EVKVITLKSKLVSDFRKDFQFYKVREINNYHHAHDAYLNAVVGTALIKKY
PKLESEFVYGDYKVYDVRKMIAKSEQEIGKATAKYFFYSNIMNFFKTEIT
LANGEIRKRPLIETNGETGEIVWDKGRDFATVRKVLSMPQVNIVKKTEVQ
TGGFSKESILPKRNSDKLIARKKDWDPKKYGGFDSPTVAYSVLVVAKVEK
GKSKKLKSVKELLGITIMERSSFEKNPIDFLEAKGYKEVKKDLIIKLPKY
SLFELENGRKRMLASAGELQKGNELALPSKYVNFLYLASHYEKLKGSPED
NEQKQLFVEQHKHYLDEIIEQISEFSKRVILADANLDKVLSAYNKHRDKP
IREQAENIIHLFTLTNLGAPAAFKYFDTTIDRKRYTSTKEVLDATLIHQS
ITGLYETRIDLSQLGGD.

Exemplary Streptococcus pyogenes Cas9 nickase (SpCas9 nickase) amino acid sequence:

(SEQ ID NO: 5785)
MDKKYSIGLDIGTNSVGWAVITDEYKVPSKKFKVLGNTDRHSIKKNLIGA
LLFDSGETAEATRLKRTARRRYTRRKNRICYLQEIFSNEMAKVDDSFFHR
LEESFLVEEDKKHERHPIFGNIVDEVAYHEKYPTIYHLRKKLVDSTDKAD
LRLIYLALAHMIKFRGHFLIEGDLNPDNSDVDKLFIQLVQTYNQLFEENP
INASGVDAKAILSARLSKSRRLENLIAQLPGEKKNGLFGNLIALSLGLTP
NFKSNFDLAEDAKLQLSKDTYDDDLDNLLAQIGDQYADLFLAAKNLSDAI
LLSDILRVNTEITKAPLSASMIKRYDEHHQDLTLLKALVRQQLPEKYKEI
FFDQSKNGYAGYIDGGASQEEFYKFIKPILEKMDGTEELLVKLNREDLLR
KQRTFDNGSIPHQIHLGELHAILRRQEDFYPFLKDNREKIEKILTFRIPY
YVGPLARGNSRFAWMTRKSEETITPWNFEEVVDKGASAQSFIERMTNFDK
NLPNEKVLPKHSLLYEYFTVYNELTKVKYVTEGMRKPAFLSGEQKKAIVD
LLFKTNRKVTVKQLKEDYFKKIECFDSVEISGVEDRFNASLGTYHDLLKI
IKDKDFLDNEENEDILEDIVLTLTLFEDREMIEERLKTYAHLFDDKVMKQ
LKRRRYTGWGRLSRKLINGIRDKQSGKTILDFLKSDGFANRNFMQLIHDD
SLTFKEDIQKAQVSGQGDSLHEHIANLAGSPAIKKGILQTVKVVDELVKV
MGRHKPENIVIEMARENQTTQKGQKNSRERMKRIEEGIKELGSQILKEHP
VENTQLQNEKLYLYYLQNGRDMYVDQELDINRLSDYDVDAIVPQSFLKDD
SIDNKVLTRSDKNRGKSDNVPSEEVVKKMKNYWRQLLNAKLITQRKFDNL
TKAERGGLSELDKAGFIKRQLVETRQITKHVAQILDSRMNTKYDENDKLI
REVKVITLKSKLVSDFRKDFQFYKVREINNYHHAHDAYLNAVVGTALIKK
YPKLESEFVYGDYKVYDVRKMIAKSEQEIGKATAKYFFYSNIMNFFKTEI
TLANGEIRKRPLIETNGETGEIVWDKGRDFATVRKVLSMPQVNIVKKTEV
QTGGFSKESILPKRNSDKLIARKKDWDPKKYGGFDSPTVAYSVLVVAKVE
KGKSKKLKSVKELLGITIMERSSFEKNPIDFLEAKGYKEVKKDLIIKLPK
YSLFELENGRKRMLASAGELQKGNELALPSKYVNFLYLASHYEKLKGSPE
DNEQKQLFVEQHKHYLDEIIEQISEFSKRVILADANLDKVLSAYNKHRDK
PIREQAENIIHLFTLTNLGAPAAFKYFDTTIDRKRYTSTKEVLDATLIHQ
SITGLYETRIDLSQLGGD.

Exemplary Streptococcus pyogenes Cas9 nickase (SpCas9 nickase) amino acid sequence lacking the N-terminus methionine:

(SEQ ID NO: 5786)
DKKYSIGLDIGTNSVGWAVITDEYKVPSKKFKVLGNTDRHSIKKNLIGAL
LFDSGETAEATRLKRTARRRYTRRKNRICYLQEIFSNEMAKVDDSFFHRL
EESFLVEEDKKHERHPIFGNIVDEVAYHEKYPTIYHLRKKLVDSTDKADL
RLIYLALAHMIKFRGHFLIEGDLNPDNSDVDKLFIQLVQTYNQLFEENPI
NASGVDAKAILSARLSKSRRLENLIAQLPGEKKNGLFGNLIALSLGLTPN
FKSNFDLAEDAKLQLSKDTYDDDLDNLLAQIGDQYADLFLAAKNLSDAIL
LSDILRVNTEITKAPLSASMIKRYDEHHQDLTLLKALVRQQLPEKYKEIF
FDQSKNGYAGYIDGGASQEEFYKFIKPILEKMDGTEELLVKLNREDLLRK
QRTFDNGSIPHQIHLGELHAILRRQEDFYPFLKDNREKIEKILTFRIPYY
VGPLARGNSRFAWMTRKSEETITPWNFEEVVDKGASAQSFIERMTNFDKN
LPNEKVLPKHSLLYEYFTVYNELTKVKYVTEGMRKPAFLSGEQKKAIVDL
LFKTNRKVTVKQLKEDYFKKIECFDSVEISGVEDRFNASLGTYHDLLKII
KDKDFLDNEENEDILEDIVLTLTLFEDREMIEERLKTYAHLFDDKVMKQL
KRRRYTGWGRLSRKLINGIRDKQSGKTILDFLKSDGFANRNFMQLIHDDS
LTFKEDIQKAQVSGQGDSLHEHIANLAGSPAIKKGILQTVKVVDELVKVM
GRHKPENIVIEMARENQTTQKGQKNSRERMKRIEEGIKELGSQILKEHPV
ENTQLQNEKLYLYYLQNGRDMYVDQELDINRLSDYDVDAIVPQSFLKDDS
IDNKVLTRSDKNRGKSDNVPSEEVVKKMKNYWRQLLNAKLITQRKFDNLT
KAERGGLSELDKAGFIKRQLVETRQITKHVAQILDSRMNTKYDENDKLIR
EVKVITLKSKLVSDFRKDFQFYKVREINNYHHAHDAYLNAVVGTALIKKY
PKLESEFVYGDYKVYDVRKMIAKSEQEIGKATAKYFFYSNIMNFFKTEIT
LANGEIRKRPLIETNGETGEIVWDKGRDFATVRKVLSMPQVNIVKKTEVQ
TGGFSKESILPKRNSDKLIARKKDWDPKKYGGFDSPTVAYSVLVVAKVEK
GKSKKLKSVKELLGITIMERSSFEKNPIDFLEAKGYKEVKKDLIIKLPKY
SLFELENGRKRMLASAGELQKGNELALPSKYVNFLYLASHYEKLKGSPED
NEQKQLFVEQHKHYLDEIIEQISEFSKRVILADANLDKVLSAYNKHRDKP
IREQAENIIHLFTLTNLGAPAAFKYFDTTIDRKRYTSTKEVLDATLIHQS
ITGLYETRIDLSQLGGD.

Exemplary Streptococcus pyogenes Cas9 (SpCas9) variant; SpCas9 NG amino acid sequence

(SEQ ID NO: 5787)
MDKKYSIGLDIGTNSVGWAVITDEYKVPSKKFKVLGNTDRHSIKKNLIGA
LLFDSGETAEATRLKRTARRRYTRRKNRICYLQEIFSNEMAKVDDSFFHR
LEESFLVEEDKKHERHPIFGNIVDEVAYHEKYPTIYHLRKKLVDSTDKAD
LRLIYLALAHMIKFRGHFLIEGDLNPDNSDVDKLFIQLVQTYNQLFEENP
INASGVDAKAILSARLSKSRRLENLIAQLPGEKKNGLFGNLIALSLGLTP
NFKSNFDLAEDAKLQLSKDTYDDDLDNLLAQIGDQYADLFLAAKNLSDAI
LLSDILRVNTEITKAPLSASMIKRYDEHHQDLTLLKALVRQQLPEKYKEI
FFDQSKNGYAGYIDGGASQEEFYKFIKPILEKMDGTEELLVKLNREDLLR
KQRTFDNGSIPHQIHLGELHAILRRQEDFYPFLKDNREKIEKILTFRIPY
YVGPLARGNSRFAWMTRKSEETITPWNFEEVVDKGASAQSFIERMTNFDK
NLPNEKVLPKHSLLYEYFTVYNELTKVKYVTEGMRKPAFLSGEQKKAIVD
LLFKTNRKVTVKQLKEDYFKKIECFDSVEISGVEDRFNASLGTYHDLLKI
IKDKDFLDNEENEDILEDIVLTLTLFEDREMIEERLKTYAHLFDDKVMKQ
LKRRRYTGWGRLSRKLINGIRDKQSGKTILDFLKSDGFANRNFMQLIHDD
SLTFKEDIQKAQVSGQGDSLHEHIANLAGSPAIKKGILQTVKVVDELVKV
MGRHKPENIVIEMARENQTTQKGQKNSRERMKRIEEGIKELGSQILKEHP
VENTQLQNEKLYLYYLQNGRDMYVDQELDINRLSDYDVDHIVPQSFLKDD
SIDNKVLTRSDKNRGKSDNVPSEEVVKKMKNYWRQLLNAKLITQRKFDNL
TKAERGGLSELDKAGFIKRQLVETRQITKHVAQILDSRMNTKYDENDKLI
REVKVITLKSKLVSDFRKDFQFYKVREINNYHHAHDAYLNAVVGTALIKK
YPKLESEFVYGDYKVYDVRKMIAKSEQEIGKATAKYFFYSNIMNFFKTEI
TLANGEIRKRPLIETNGETGEIVWDKGRDFATVRKVLSMPQVNIVKKTEV
QTGGFSKESIRPKRNSDKLIARKKDWDPKKYGGFVSPTVAYSVLVVAKVE
KGKSKKLKSVKELLGITIMERSSFEKNPIDFLEAKGYKEVKKDLIIKLPK
YSLFELENGRKRMLASARFLQKGNELALPSKYVNFLYLASHYEKLKGSPE
DNEQKQLFVEQHKHYLDEIIEQISEFSKRVILADANLDKVLSAYNKHRDK
PIREQAENIIHLFTLTNLGAPRAFKYFDTTIDRKVYRSTKEVLDATLIHQ
SITGLYETRIDLSQLGGD.

Exemplary Streptococcus pyogenes Cas9 (SpCas9) variant; SpCas9 NG amino acid sequence lacking the N-terminus methionine:

(SEQ ID NO: 5788)
DKKYSIGLDIGTNSVGWAVITDEYKVPSKKFKVLGNTDRHSIKKNLIGAL
LFDSGETAEATRLKRTARRRYTRRKNRICYLQEIFSNEMAKVDDSFFHRL
EESFLVEEDKKHERHPIFGNIVDEVAYHEKYPTIYHLRKKLVDSTDKADL
RLIYLALAHMIKFRGHFLIEGDLNPDNSDVDKLFIQLVQTYNQLFEENPI
NASGVDAKAILSARLSKSRRLENLIAQLPGEKKNGLFGNLIALSLGLTPN
FKSNFDLAEDAKLQLSKDTYDDDLDNLLAQIGDQYADLFLAAKNLSDAIL
LSDILRVNTEITKAPLSASMIKRYDEHHQDLTLLKALVRQQLPEKYKEIF
FDQSKNGYAGYIDGGASQEEFYKFIKPILEKMDGTEELLVKLNREDLLRK
QRTFDNGSIPHQIHLGELHAILRRQEDFYPFLKDNREKIEKILTFRIPYY
VGPLARGNSRFAWMTRKSEETITPWNFEEVVDKGASAQSFIERMTNFDKN
LPNEKVLPKHSLLYEYFTVYNELTKVKYVTEGMRKPAFLSGEQKKAIVDL
LFKTNRKVTVKQLKEDYFKKIECFDSVEISGVEDRFNASLGTYHDLLKII
KDKDFLDNEENEDILEDIVLTLTLFEDREMIEERLKTYAHLFDDKVMKQL
KRRRYTGWGRLSRKLINGIRDKQSGKTILDFLKSDGFANRNFMQLIHDDS
LTFKEDIQKAQVSGQGDSLHEHIANLAGSPAIKKGILQTVKVVDELVKVM
GRHKPENIVIEMARENQTTQKGQKNSRERMKRIEEGIKELGSQILKEHPV
ENTQLQNEKLYLYYLQNGRDMYVDQELDINRLSDYDVDHIVPQSFLKDDS
IDNKVLTRSDKNRGKSDNVPSEEVVKKMKNYWRQLLNAKLITQRKFDNLT
KAERGGLSELDKAGFIKRQLVETRQITKHVAQILDSRMNTKYDENDKLIR
EVKVITLKSKLVSDFRKDFQFYKVREINNYHHAHDAYLNAVVGTALIKKY
PKLESEFVYGDYKVYDVRKMIAKSEQEIGKATAKYFFYSNIMNFFKTEIT
LANGEIRKRPLIETNGETGEIVWDKGRDFATVRKVLSMPQVNIVKKTEVQ
TGGFSKESIRPKRNSDKLIARKKDWDPKKYGGFVSPTVAYSVLVVAKVEK
GKSKKLKSVKELLGITIMERSSFEKNPIDFLEAKGYKEVKKDLIIKLPKY
SLFELENGRKRMLASARFLQKGNELALPSKYVNFLYLASHYEKLKGSPED
NEQKQLFVEQHKHYLDEIIEQISEFSKRVILADANLDKVLSAYNKHRDKP
IREQAENIIHLFTLTNLGAPRAFKYFDTTIDRKVYRSTKEVLDATLIHQS
ITGLYETRIDLSQLGGD.

Exemplary Streptococcus pyogenes Cas9 (SpCas9) nickase; SpCas9 NG nickase amino acid sequence:

(SEQ ID NO: 5789)
MDKKYSIGLDIGTNSVGWAVITDEYKVPSKKFKVLGNTDRHSIKKNLIGA
LLFDSGETAEATRLKRTARRRYTRRKNRICYLQEIFSNEMAKVDDSFFHR
LEESFLVEEDKKHERHPIFGNIVDEVAYHEKYPTIYHLRKKLVDSTDKAD
LRLIYLALAHMIKFRGHFLIEGDLNPDNSDVDKLFIQLVQTYNQLFEENP
INASGVDAKAILSARLSKSRRLENLIAQLPGEKKNGLFGNLIALSLGLTP
NFKSNFDLAEDAKLQLSKDTYDDDLDNLLAQIGDQYADLFLAAKNLSDAI
LLSDILRVNTEITKAPLSASMIKRYDEHHQDLTLLKALVRQQLPEKYKEI
FFDQSKNGYAGYIDGGASQEEFYKFIKPILEKMDGTEELLVKLNREDLLR
KQRTFDNGSIPHQIHLGELHAILRRQEDFYPFLKDNREKIEKILTFRIPY
YVGPLARGNSRFAWMTRKSEETITPWNFEEVVDKGASAQSFIERMTNFDK
NLPNEKVLPKHSLLYEYFTVYNELTKVKYVTEGMRKPAFLSGEQKKAIVD
LLFKTNRKVTVKQLKEDYFKKIECFDSVEISGVEDRFNASLGTYHDLLKI
IKDKDFLDNEENEDILEDIVLTLTLFEDREMIEERLKTYAHLFDDKVMKQ
LKRRRYTGWGRLSRKLINGIRDKQSGKTILDFLKSDGFANRNFMQLIHDD
SLTFKEDIQKAQVSGQGDSLHEHIANLAGSPAIKKGILQTVKVVDELVKV
MGRHKPENIVIEMARENQTTQKGQKNSRERMKRIEEGIKELGSQILKEHP
VENTQLQNEKLYLYYLQNGRDMYVDQELDINRLSDYDVDAIVPQSFLKDD
SIDNKVLTRSDKNRGKSDNVPSEEVVKKMKNYWRQLLNAKLITQRKFDNL
TKAERGGLSELDKAGFIKRQLVETRQITKHVAQILDSRMNTKYDENDKLI
REVKVITLKSKLVSDFRKDFQFYKVREINNYHHAHDAYLNAVVGTALIKK
YPKLESEFVYGDYKVYDVRKMIAKSEQEIGKATAKYFFYSNIMNFFKTEI
TLANGEIRKRPLIETNGETGEIVWDKGRDFATVRKVLSMPQVNIVKKTEV
QTGGFSKESIRPKRNSDKLIARKKDWDPKKYGGFVSPTVAYSVLVVAKVE
KGKSKKLKSVKELLGITIMERSSFEKNPIDFLEAKGYKEVKKDLIIKLPK
YSLFELENGRKRMLASARFLQKGNELALPSKYVNFLYLASHYEKLKGSPE
DNEQKQLFVEQHKHYLDEIIEQISEFSKRVILADANLDKVLSAYNKHRDK
PIREQAENIIHLFTLTNLGAPRAFKYFDTTIDRKVYRSTKEVLDATLIHQ
SITGLYETRIDLSQLGGD.

Exemplary Streptococcus pyogenes Cas9 (SpCas9) nickase; SpCas9 NG nickase amino acid sequence lacking the N-terminus methionine:

(SEQ ID NO: 5790)
DKKYSIGLDIGTNSVGWAVITDEYKVPSKKFKVLGNTDRHSIKKNLIGAL
LFDSGETAEATRLKRTARRRYTRRKNRICYLQEIFSNEMAKVDDSFFHRL
EESFLVEEDKKHERHPIFGNIVDEVAYHEKYPTIYHLRKKLVDSTDKADL
RLIYLALAHMIKFRGHFLIEGDLNPDNSDVDKLFIQLVQTYNQLFEENPI
NASGVDAKAILSARLSKSRRLENLIAQLPGEKKNGLFGNLIALSLGLTPN
FKSNFDLAEDAKLQLSKDTYDDDLDNLLAQIGDQYADLFLAAKNLSDAIL
LSDILRVNTEITKAPLSASMIKRYDEHHQDLTLLKALVRQQLPEKYKEIF
FDQSKNGYAGYIDGGASQEEFYKFIKPILEKMDGTEELLVKLNREDLLRK
QRTFDNGSIPHQIHLGELHAILRRQEDFYPFLKDNREKIEKILTFRIPYY
VGPLARGNSRFAWMTRKSEETITPWNFEEVVDKGASAQSFIERMTNFDKN
LPNEKVLPKHSLLYEYFTVYNELTKVKYVTEGMRKPAFLSGEQKKAIVDL
LFKTNRKVTVKQLKEDYFKKIECFDSVEISGVEDRFNASLGTYHDLLKII
KDKDFLDNEENEDILEDIVLTLTLFEDREMIEERLKTYAHLFDDKVMKQL
KRRRYTGWGRLSRKLINGIRDKQSGKTILDFLKSDGFANRNFMQLIHDDS
LTFKEDIQKAQVSGQGDSLHEHIANLAGSPAIKKGILQTVKVVDELVKVM
GRHKPENIVIEMARENQTTQKGQKNSRERMKRIEEGIKELGSQILKEHPV
ENTQLQNEKLYLYYLQNGRDMYVDQELDINRLSDYDVDAIVPQSFLKDDS
IDNKVLTRSDKNRGKSDNVPSEEVVKKMKNYWRQLLNAKLITQRKFDNLT
KAERGGLSELDKAGFIKRQLVETRQITKHVAQILDSRMNTKYDENDKLIR
EVKVITLKSKLVSDFRKDFQFYKVREINNYHHAHDAYLNAVVGTALIKKY
PKLESEFVYGDYKVYDVRKMIAKSEQEIGKATAKYFFYSNIMNFFKTEIT
LANGEIRKRPLIETNGETGEIVWDKGRDFATVRKVLSMPQVNIVKKTEVQ
TGGFSKESIRPKRNSDKLIARKKDWDPKKYGGFVSPTVAYSVLVVAKVEK
GKSKKLKSVKELLGITIMERSSFEKNPIDFLEAKGYKEVKKDLIIKLPKY
SLFELENGRKRMLASARFLQKGNELALPSKYVNFLYLASHYEKLKGSPED
NEQKQLFVEQHKHYLDEIIEQISEFSKRVILADANLDKVLSAYNKHRDKP
IREQAENIIHLFTLTNLGAPRAFKYFDTTIDRKVYRSTKEVLDATLIHQS
ITGLYETRIDLSQLGGD.

Exemplary Streptococcus pyogenes Cas9 (SpCas9) variant; SpCas9 VRQR amino acid sequence:

(SEQ ID NO: 5791)
MDKKYSIGLDIGTNSVGWAVITDEYKVPSKKFKVLGNTDRHSIKKNLIGA
LLFDSGETAEATRLKRTARRRYTRRKNRICYLQEIFSNEMAKVDDSFFHR
LEESFLVEEDKKHERHPIFGNIVDEVAYHEKYPTIYHLRKKLVDSTDKAD
LRLIYLALAHMIKFRGHFLIEGDLNPDNSDVDKLFIQLVQTYNQLFEENP
INASGVDAKAILSARLSKSRRLENLIAQLPGEKKNGLFGNLIALSLGLTP
NFKSNFDLAEDAKLQLSKDTYDDDLDNLLAQIGDQYADLFLAAKNLSDAI
LLSDILRVNTEITKAPLSASMIKRYDEHHQDLTLLKALVRQQLPEKYKEI
FFDQSKNGYAGYIDGGASQEEFYKFIKPILEKMDGTEELLVKLNREDLLR
KQRTFDNGSIPHQIHLGELHAILRRQEDFYPFLKDNREKIEKILTFRIPY
YVGPLARGNSRFAWMTRKSEETITPWNFEEVVDKGASAQSFIERMTNFDK
NLPNEKVLPKHSLLYEYFTVYNELTKVKYVTEGMRKPAFLSGEQKKAIVD
LLFKTNRKVTVKQLKEDYFKKIECFDSVEISGVEDRFNASLGTYHDLLKI
IKDKDFLDNEENEDILEDIVLTLTLFEDREMIEERLKTYAHLFDDKVMKQ
LKRRRYTGWGRLSRKLINGIRDKQSGKTILDFLKSDGFANRNFMQLIHDD
SLTFKEDIQKAQVSGQGDSLHEHIANLAGSPAIKKGILQTVKVVDELVKV
MGRHKPENIVIEMARENQTTQKGQKNSRERMKRIEEGIKELGSQILKEHP
VENTQLQNEKLYLYYLQNGRDMYVDQELDINRLSDYDVDHIVPQSFLKDD
SIDNKVLTRSDKNRGKSDNVPSEEVVKKMKNYWRQLLNAKLITQRKFDNL
TKAERGGLSELDKAGFIKRQLVETRQITKHVAQILDSRMNTKYDENDKLI
REVKVITLKSKLVSDFRKDFQFYKVREINNYHHAHDAYLNAVVGTALIKK
YPKLESEFVYGDYKVYDVRKMIAKSEQEIGKATAKYFFYSNIMNFFKTEI
TLANGEIRKRPLIETNGETGEIVWDKGRDFATVRKVLSMPQVNIVKKTEV
QTGGFSKESILPKRNSDKLIARKKDWDPKKYGGFVSPTVAYSVLVVAKVE
KGKSKKLKSVKELLGITIMERSSFEKNPIDFLEAKGYKEVKKDLIIKLPK
YSLFELENGRKRMLASARELQKGNELALPSKYVNFLYLASHYEKLKGSPE
DNEQKQLFVEQHKHYLDEIIEQISEFSKRVILADANLDKVLSAYNKHRDK
PIREQAENIIHLFTLTNLGAPAAFKYFDTTIDRKQYRSTKEVLDATLIHQ
SITGLYETRIDLSQLGGD.

Exemplary Streptococcus pyogenes Cas9 (SpCas9) variant; SpCas9 VRQR amino acid sequence lacking the N-terminus methionine:

(SEQ ID NO: 5792)
DKKYSIGLDIGTNSVGWAVITDEYKVPSKKFKVLGNTDRHSIKKNLIGAL
LFDSGETAEATRLKRTARRRYTRRKNRICYLQEIFSNEMAKVDDSFFHRL
EESFLVEEDKKHERHPIFGNIVDEVAYHEKYPTIYHLRKKLVDSTDKADL
RLIYLALAHMIKFRGHFLIEGDLNPDNSDVDKLFIQLVQTYNQLFEENPI
NASGVDAKAILSARLSKSRRLENLIAQLPGEKKNGLFGNLIALSLGLTPN
FKSNFDLAEDAKLQLSKDTYDDDLDNLLAQIGDQYADLFLAAKNLSDAIL
LSDILRVNTEITKAPLSASMIKRYDEHHQDLTLLKALVRQQLPEKYKEIF
FDQSKNGYAGYIDGGASQEEFYKFIKPILEKMDGTEELLVKLNREDLLRK
QRTFDNGSIPHQIHLGELHAILRRQEDFYPFLKDNREKIEKILTFRIPYY
VGPLARGNSRFAWMTRKSEETITPWNFEEVVDKGASAQSFIERMTNFDKN
LPNEKVLPKHSLLYEYFTVYNELTKVKYVTEGMRKPAFLSGEQKKAIVDL
LFKTNRKVTVKQLKEDYFKKIECFDSVEISGVEDRFNASLGTYHDLLKII
KDKDFLDNEENEDILEDIVLTLTLFEDREMIEERLKTYAHLFDDKVMKQL
KRRRYTGWGRLSRKLINGIRDKQSGKTILDFLKSDGFANRNFMQLIHDDS
LTFKEDIQKAQVSGQGDSLHEHIANLAGSPAIKKGILQTVKVVDELVKVM
GRHKPENIVIEMARENQTTQKGQKNSRERMKRIEEGIKELGSQILKEHPV
ENTQLQNEKLYLYYLQNGRDMYVDQELDINRLSDYDVDHIVPQSFLKDDS
IDNKVLTRSDKNRGKSDNVPSEEVVKKMKNYWRQLLNAKLITQRKFDNLT
KAERGGLSELDKAGFIKRQLVETRQITKHVAQILDSRMNTKYDENDKLIR
EVKVITLKSKLVSDFRKDFQFYKVREINNYHHAHDAYLNAVVGTALIKKY
PKLESEFVYGDYKVYDVRKMIAKSEQEIGKATAKYFFYSNIMNFFKTEIT
LANGEIRKRPLIETNGETGEIVWDKGRDFATVRKVLSMPQVNIVKKTEVQ
TGGFSKESILPKRNSDKLIARKKDWDPKKYGGFVSPTVAYSVLVVAKVEK
GKSKKLKSVKELLGITIMERSSFEKNPIDFLEAKGYKEVKKDLIIKLPKY
SLFELENGRKRMLASARELQKGNELALPSKYVNFLYLASHYEKLKGSPED
NEQKQLFVEQHKHYLDEIIEQISEFSKRVILADANLDKVLSAYNKHRDKP
IREQAENIIHLFTLTNLGAPAAFKYFDTTIDRKQYRSTKEVLDATLIHQS
ITGLYETRIDLSQLGGD.

Exemplary Streptococcus pyogenes Cas9 (SpCas9) nickase; SpCas9 VRQR nickase amino acid sequence:

(SEQ ID NO: 5793)
MDKKYSIGLDIGTNSVGWAVITDEYKVPSKKFKVLGNTDRHSIKKNLIGA
LLFDSGETAEATRLKRTARRRYTRRKNRICYLQEIFSNEMAKVDDSFFHR
LEESFLVEEDKKHERHPIFGNIVDEVAYHEKYPTIYHLRKKLVDSTDKAD
LRLIYLALAHMIKFRGHFLIEGDLNPDNSDVDKLFIQLVQTYNQLFEENP
INASGVDAKAILSARLSKSRRLENLIAQLPGEKKNGLFGNLIALSLGLTP
NFKSNFDLAEDAKLQLSKDTYDDDLDNLLAQIGDQYADLFLAAKNLSDAI
LLSDILRVNTEITKAPLSASMIKRYDEHHQDLTLLKALVRQQLPEKYKEI
FFDQSKNGYAGYIDGGASQEEFYKFIKPILEKMDGTEELLVKLNREDLLR
KQRTFDNGSIPHQIHLGELHAILRRQEDFYPFLKDNREKIEKILTFRIPY
YVGPLARGNSRFAWMTRKSEETITPWNFEEVVDKGASAQSFIERMTNFDK
NLPNEKVLPKHSLLYEYFTVYNELTKVKYVTEGMRKPAFLSGEQKKAIVD
LLFKTNRKVTVKQLKEDYFKKIECFDSVEISGVEDRFNASLGTYHDLLKI
IKDKDFLDNEENEDILEDIVLTLTLFEDREMIEERLKTYAHLFDDKVMKQ
LKRRRYTGWGRLSRKLINGIRDKQSGKTILDFLKSDGFANRNFMQLIHDD
SLTFKEDIQKAQVSGQGDSLHEHIANLAGSPAIKKGILQTVKVVDELVKV
MGRHKPENIVIEMARENQTTQKGQKNSRERMKRIEEGIKELGSQILKEHP
VENTQLQNEKLYLYYLQNGRDMYVDQELDINRLSDYDVDAIVPQSFLKDD
SIDNKVLTRSDKNRGKSDNVPSEEVVKKMKNYWRQLLNAKLITQRKFDNL
TKAERGGLSELDKAGFIKRQLVETRQITKHVAQILDSRMNTKYDENDKLI
REVKVITLKSKLVSDFRKDFQFYKVREINNYHHAHDAYLNAVVGTALIKK
YPKLESEFVYGDYKVYDVRKMIAKSEQEIGKATAKYFFYSNIMNFFKTEI
TLANGEIRKRPLIETNGETGEIVWDKGRDFATVRKVLSMPQVNIVKKTEV
QTGGFSKESILPKRNSDKLIARKKDWDPKKYGGFVSPTVAYSVLVVAKVE
KGKSKKLKSVKELLGITIMERSSFEKNPIDFLEAKGYKEVKKDLIIKLPK
YSLFELENGRKRMLASARELQKGNELALPSKYVNFLYLASHYEKLKGSPE
DNEQKQLFVEQHKHYLDEIIEQISEFSKRVILADANLDKVLSAYNKHRDK
PIREQAENIIHLFTLTNLGAPAAFKYFDTTIDRKQYRSTKEVLDATLIHQ
SITGLYETRIDLSQLGGD.

Exemplary Streptococcus pyogenes Cas9 (SpCas9) nickase; SpCas9 VRQR nickase amino acid sequence lacking the N-terminus methionine:

(SEQ ID NO: 5794)
DKKYSIGLDIGTNSVGWAVITDEYKVPSKKFKVLGNTDRHSIKKNLIGAL
LFDSGETAEATRLKRTARRRYTRRKNRICYLQEIFSNEMAKVDDSFFHRL
EESFLVEEDKKHERHPIFGNIVDEVAYHEKYPTIYHLRKKLVDSTDKADL
RLIYLALAHMIKFRGHFLIEGDLNPDNSDVDKLFIQLVQTYNQLFEENPI
NASGVDAKAILSARLSKSRRLENLIAQLPGEKKNGLFGNLIALSLGLTPN
FKSNFDLAEDAKLQLSKDTYDDDLDNLLAQIGDQYADLFLAAKNLSDAIL
LSDILRVNTEITKAPLSASMIKRYDEHHQDLTLLKALVRQQLPEKYKEIF
FDQSKNGYAGYIDGGASQEEFYKFIKPILEKMDGTEELLVKLNREDLLRK
QRTFDNGSIPHQIHLGELHAILRRQEDFYPFLKDNREKIEKILTFRIPYY
VGPLARGNSRFAWMTRKSEETITPWNFEEVVDKGASAQSFIERMTNFDKN
LPNEKVLPKHSLLYEYFTVYNELTKVKYVTEGMRKPAFLSGEQKKAIVDL
LFKTNRKVTVKQLKEDYFKKIECFDSVEISGVEDRFNASLGTYHDLLKII
KDKDFLDNEENEDILEDIVLTLTLFEDREMIEERLKTYAHLFDDKVMKQL
KRRRYTGWGRLSRKLINGIRDKQSGKTILDFLKSDGFANRNFMQLIHDDS
LTFKEDIQKAQVSGQGDSLHEHIANLAGSPAIKKGILQTVKVVDELVKVM
GRHKPENIVIEMARENQTTQKGQKNSRERMKRIEEGIKELGSQILKEHPV
ENTQLQNEKLYLYYLQNGRDMYVDQELDINRLSDYDVDAIVPQSFLKDDS
IDNKVLTRSDKNRGKSDNVPSEEVVKKMKNYWRQLLNAKLITQRKFDNLT
KAERGGLSELDKAGFIKRQLVETRQITKHVAQILDSRMNTKYDENDKLIR
EVKVITLKSKLVSDFRKDFQFYKVREINNYHHAHDAYLNAVVGTALIKKY
PKLESEFVYGDYKVYDVRKMIAKSEQEIGKATAKYFFYSNIMNFFKTEIT
LANGEIRKRPLIETNGETGEIVWDKGRDFATVRKVLSMPQVNIVKKTEVQ
TGGFSKESILPKRNSDKLIARKKDWDPKKYGGFVSPTVAYSVLVVAKVEK
GKSKKLKSVKELLGITIMERSSFEKNPIDFLEAKGYKEVKKDLIIKLPKY
SLFELENGRKRMLASARELQKGNELALPSKYVNFLYLASHYEKLKGSPED
NEQKQLFVEQHKHYLDEIIEQISEFSKRVILADANLDKVLSAYNKHRDKP
IREQAENIIHLFTLTNLGAPAAFKYFDTTIDRKQYRSTKEVLDATLIHQS
ITGLYETRIDLSQLGGD.

In some embodiments, a prime editor comprises a Cas9 protein as according to any of the SEQ ID NOS 5795-5798 or a variant thereof. In some embodiments, a prime editor comprises a Cas9 protein from Staphylococcus lugdunensis (Slu Cas9) e.g., as according to any of the SEQ ID NOS 5795-5798 or a variant thereof. In some embodiments, a sluCas9 lacks a N-terminal methionine relative to a corresponding sluCas9 (e.g., a wild type sluCas9, a sluCas9 variant, or a nickase sluCas9). In some embodiments, the Cas9 protein is a sluCas9. In some embodiments, a sluCas9 can be a wild type sluCas9, a sluCas9 variant or a nickase sluCas9. In some embodiments, a prime editor comprises a Cas9 protein, having an amino acid sequence as according to SEQ ID NO: 5795, not including the N-terminus methionine. In some embodiments, a wild type SluCas9 comprises an amino acid sequence set forth in SEQ ID NO: 5795 or SEQ ID NO: 5796. In some embodiments, a prime editor comprises a Cas9 protein, having an amino acid sequence as according to SEQ ID NO: 5795, not including the N-terminus methionine (e.g., as set forth in SEQ ID NO: 5796). In some embodiments, a prime editor comprises a Cas9 protein comprising one or more mutations (e.g., amino acid substitutions, insertions and/or deletions relative to a corresponding wild type Cas9 protein (e.g., wild type sluCas9). In some embodiments, the Cas9 protein comprising one or mutations relative to a wild type Cas9 protein comprises an amino acid sequence set forth in SEQ ID NO: 5797 or 5798.

Exemplary Staphylococcus lugdunensis Cas9 (SluCas9) amino acid sequences useful in the prime editors disclosed herein are provided below in SEQ ID NOs: 5795-5798.

Exemplary Staphylococcus lugdunensis amino acid sequence WP_002460848.1.

Exemplary wild type Staphylococcus lugdunensis Cas9 (SluCas9) amino acid sequence:

(SEQ ID NO: 5795)
MNQKFILGLDIGITSVGYGLIDYETKNIIDAGVRLFPEANVENNEGRRSK
RGSRRLKRRRIHRLERVKKLLEDYNLLDQSQIPQSTNPYAIRVKGLSEAL
SKDELVIALLHIAKRRGIHKIDVIDSNDDVGNELSTKEQLNKNSKLLKDK
FVCQIQLERMNEGQVRGEKNRFKTADIIKEIIQLLNVQKNFHQLDENFIN
KYIELVEMRREYFEGPGKGSPYGWEGDPKAWYETLMGHCTYFPDELRSVK
YAYSADLFNALNDLNNLVIQRDGLSKLEYHEKYHIIENVFKQKKKPTLKQ
IANEINVNPEDIKGYRITKSGKPQFTEFKLYHDLKSVLFDQSILENEDVL
DQIAEILTIYQDKDSIKSKLTELDILLNEEDKENIAQLTGYTGTHRLSLK
CIRLVLEEQWYSSRNQMEIFTHLNIKPKKINLTAANKIPKAMIDEFILSP
VVKRTFGQAINLINKIIEKYGVPEDIIIELARENNSKDKQKFINEMQKKN
ENTRKRINEIIGKYGNQNAKRLVEKIRLHDEQEGKCLYSLESIPLEDLLN
NPNHYEVDHIIPRSVSFDNSYHNKVLVKQSENSKKSNLTPYQYFNSGKSK
LSYNQFKQHILNLSKSQDRISKKKKEYLLEERDINKFEVQKEFINRNLVD
TRYATRELTNYLKAYFSANNMNVKVKTINGSFTDYLRKVWKFKKERNHGY
KHHAEDALIIANADFLFKENKKLKAVNSVLEKPEIESKQLDIQVDSEDNY
SEMFIIPKQVQDIKDFRNFKYSHRVDKKPNRQLINDTLYSTRKKDNSTYI
VQTIKDIYAKDNTTLKKQFDKSPEKFLMYQHDPRTFEKLEVIMKQYANEK
NPLAKYHEETGEYLTKYSKKNNGPIVKSLKYIGNKLGSHLDVTHQFKSST
KKLVKLSIKPYRFDVYLTDKGYKFITISYLDVLKKDNYYYIPEQKYDKLK
LGKAIDKNAKFIASFYKNDLIKLDGEIYKIIGVNSDTRNMIELDLPDIRY
KEYCELNNIKGEPRIKKTIGKKVNSIEKLTTDVLGNVFTNTQYTKPQLLF
KRGN.

Exemplary wild type Staphylococcus lugdunensis Cas9 (SluCas9) amino acid sequence lacking N-terminus methionine:

(SEQ ID NO: 5796)
NQKFILGLDIGITSVGYGLIDYETKNIIDAGVRLFPEANVENNEGRRSKR
GSRRLKRRRIHRLERVKKLLEDYNLLDQSQIPQSTNPYAIRVKGLSEALS
KDELVIALLHIAKRRGIHKIDVIDSNDDVGNELSTKEQLNKNSKLLKDKF
VCQIQLERMNEGQVRGEKNRFKTADIIKEIIQLLNVQKNFHOLDENFINK
YIELVEMRREYFEGPGKGSPYGWEGDPKAWYETLMGHCTYFPDELRSVKY
AYSADLFNALNDLNNLVIQRDGLSKLEYHEKYHIIENVFKQKKKPTLKQI
ANEINVNPEDIKGYRITKSGKPQFTEFKLYHDLKSVLFDQSILENEDVLD
QIAEILTIYQDKDSIKSKLTELDILLNEEDKENIAQLTGYTGTHRLSLKC
IRLVLEEQWYSSRNQMEIFTHLNIKPKKINLTAANKIPKAMIDEFILSPV
VKRTFGQAINLINKIIEKYGVPEDIIIELARENNSKDKQKFINEMQKKNE
NTRKRINEIIGKYGNQNAKRLVEKIRLHDEQEGKCLYSLESIPLEDLLNN
PNHYEVDHIIPRSVSFDNSYHNKVLVKQSENSKKSNLTPYQYFNSGKSKL
SYNQFKQHILNLSKSQDRISKKKKEYLLEERDINKFEVQKEFINRNLVDT
RYATRELTNYLKAYFSANNMNVKVKTINGSFTDYLRKVWKFKKERNHGYK
HHAEDALIIANADFLFKENKKLKAVNSVLEKPEIESKQLDIQVDSEDNYS
EMFIIPKQVQDIKDFRNFKYSHRVDKKPNRQLINDTLYSTRKKDNSTYIV
QTIKDIYAKDNTTLKKQFDKSPEKFLMYQHDPRTFEKLEVIMKQYANEKN
PLAKYHEETGEYLTKYSKKNNGPIVKSLKYIGNKLGSHLDVTHQFKSSTK
KLVKLSIKPYRFDVYLTDKGYKFITISYLDVLKKDNYYYIPEQKYDKLKL
GKAIDKNAKFIASFYKNDLIKLDGEIYKIIGVNSDTRNMIELDLPDIRYK
EYCELNNIKGEPRIKKTIGKKVNSIEKLTTDVLGNVFTNTQYTKPQLLFK
RGN.

Exemplary Staphylococcus lugdunensis Cas9 (SluCas9) nickase amino acid sequence:

(SEQ ID NO: 5797)
MNQKFILGLDIGITSVGYGLIDYETKNIIDAGVRLFPEANVENNEGRRSK
RGSRRLKRRRIHRLERVKKLLEDYNLLDQSQIPQSTNPYAIRVKGLSEAL
SKDELVIALLHIAKRRGIHKIDVIDSNDDVGNELSTKEQLNKNSKLLKDK
FVCQIQLERMNEGQVRGEKNRFKTADIIKEIIQLLNVQKNFHQLDENFIN
KYIELVEMRREYFEGPGKGSPYGWEGDPKAWYETLMGHCTYFPDELRSVK
YAYSADLFNALNDLNNLVIQRDGLSKLEYHEKYHIIENVFKQKKKPTLKQ
IANEINVNPEDIKGYRITKSGKPQFTEFKLYHDLKSVLFDQSILENEDVL
DQIAEILTIYQDKDSIKSKLTELDILLNEEDKENIAQLTGYTGTHRLSLK
CIRLVLEEQWYSSRNQMEIFTHLNIKPKKINLTAANKIPKAMIDEFILSP
VVKRTFGQAINLINKIIEKYGVPEDIIIELARENNSKDKQKFINEMQKKN
ENTRKRINEIIGKYGNQNAKRLVEKIRLHDEQEGKCLYSLESIPLEDLLN
NPNHYEVDHIIPRSVSFDNSYHNKVLVKQSEASKKSNLTPYQYFNSGKSK
LSYNQFKQHILNLSKSQDRISKKKKEYLLEERDINKFEVQKEFINRNLVD
TRYATRELTNYLKAYFSANNMNVKVKTINGSFTDYLRKVWKFKKERNHGY
KHHAEDALIIANADFLFKENKKLKAVNSVLEKPEIESKQLDIQVDSEDNY
SEMFIIPKQVQDIKDFRNFKYSHRVDKKPNRQLINDTLYSTRKKDNSTYI
VQTIKDIYAKDNTTLKKQFDKSPEKFLMYQHDPRTFEKLEVIMKQYANEK
NPLAKYHEETGEYLTKYSKKNNGPIVKSLKYIGNKLGSHLDVTHQFKSST
KKLVKLSIKPYRFDVYLTDKGYKFITISYLDVLKKDNYYYIPEQKYDKLK
LGKAIDKNAKFIASFYKNDLIKLDGEIYKIIGVNSDTRNMIELDLPDIRY
KEYCELNNIKGEPRIKKTIGKKVNSIEKLTTDVLGNVFTNTQYTKPQLLF
KRGN.

Exemplary Staphylococcus lugdunensis Cas9 (SluCas9) nickase amino acid sequence lacking N-terminus methionine:

(SEQ ID NO: 5798)
NQKFILGLDIGITSVGYGLIDYETKNIIDAGVRLFPEANVENNEGRRSKR
GSRRLKRRRIHRLERVKKLLEDYNLLDQSQIPQSTNPYAIRVKGLSEALS
KDELVIALLHIAKRRGIHKIDVIDSNDDVGNELSTKEQLNKNSKLLKDKF
VCQIQLERMNEGQVRGEKNRFKTADIIKEIIQLLNVQKNFHOLDENFINK
YIELVEMRREYFEGPGKGSPYGWEGDPKAWYETLMGHCTYFPDELRSVKY
AYSADLFNALNDLNNLVIQRDGLSKLEYHEKYHIIENVFKQKKKPTLKQI
ANEINVNPEDIKGYRITKSGKPQFTEFKLYHDLKSVLFDQSILENEDVLD
QIAEILTIYQDKDSIKSKLTELDILLNEEDKENIAQLTGYTGTHRLSLKC
IRLVLEEQWYSSRNQMEIFTHLNIKPKKINLTAANKIPKAMIDEFILSPV
VKRTFGQAINLINKIIEKYGVPEDIIIELARENNSKDKQKFINEMQKKNE
NTRKRINEIIGKYGNQNAKRLVEKIRLHDEQEGKCLYSLESIPLEDLLNN
PNHYEVDHIIPRSVSFDNSYHNKVLVKQSEASKKSNLTPYQYFNSGKSKL
SYNQFKQHILNLSKSQDRISKKKKEYLLEERDINKFEVQKEFINRNLVDT
RYATRELTNYLKAYFSANNMNVKVKTINGSFTDYLRKVWKFKKERNHGYK
HHAEDALIIANADFLFKENKKLKAVNSVLEKPEIESKQLDIQVDSEDNYS
EMFIIPKQVQDIKDFRNFKYSHRVDKKPNRQLINDTLYSTRKKDNSTYIV
QTIKDIYAKDNTTLKKQFDKSPEKFLMYQHDPRTFEKLEVIMKQYANEKN
PLAKYHEETGEYLTKYSKKNNGPIVKSLKYIGNKLGSHLDVTHQFKSSTK
KLVKLSIKPYRFDVYLTDKGYKFITISYLDVLKKDNYYYIPEQKYDKLKL
GKAIDKNAKFIASFYKNDLIKLDGEIYKIIGVNSDTRNMIELDLPDIRYK
EYCELNNIKGEPRIKKTIGKKVNSIEKLTTDVLGNVFTNTQYTKPQLLFK
RGN.

In some embodiments, a prime editor comprises a Cas9 protein from Staphylococcus aureus (SaCas9) e.g., as according to any of the SEQ ID NOS 5799-5800, 5802, 5803, or a variant thereof. In some embodiments, a SaCas9 may lack a N-terminal methionine. In some embodiments, a SaCas9 may comprise a mutation.

In some embodiments, a prime editor comprises a Cas9 protein as according to any of the SEQ ID NOS 5799-5800, 5802, 5803 or a variant thereof. In some embodiments, a SaCas9 lacks a N-terminal methionine relative to a corresponding SaCas9 (e.g., a wild type SaCas9, a SaCas9 variant, or a nickase SaCas9). In some embodiments, the Cas9 protein is a SaCas9. In some embodiments, a SaCas9 can be a wild type SaCas9, a SaCas9 variant or a nickase SaCas9. In some embodiments, a prime editor comprises a Cas9 protein, having an amino acid sequence as according to SEQ ID NO: 5799, not including the N-terminus methionine. In some embodiments, a wild type SaCas9 comprises an amino acid sequence set forth in SEQ ID NO: 5799 or SEQ ID NO: 5800. In some embodiments, a prime editor comprises a Cas9 protein, having an amino acid sequence as according to SEQ ID NO: 5799, not including the N-terminus methionine (e.g., as set forth in SEQ ID NO: 5800). In some embodiments, a prime editor comprises a Cas9 protein comprising one or more mutations (e.g., amino acid substitutions, insertions and/or deletions relative to a corresponding wild type Cas9 protein (e.g., wild type SaCas9). In some embodiments, the Cas9 protein comprising one or mutations relative to a wild type Cas9 protein comprises an amino acid sequence set forth in SEQ ID NO: 5802 or 5803. Exemplary SaCas9 amino acid sequences useful in the prime editors disclosed herein are provided below in SEQ ID NOs: 5799, 5800, 5802, 5803.

Exemplary wild type Staphylococcus aureus Cas9 (SaCas9) amino acid sequence:

(SEQ ID NO: 5799)
MKRNYILGLDIGITSVGYGIIDYETRDVIDAGVRLFKEANVENNEGRRSK
RGARRLKRRRRHRIQRVKKLLFDYNLLTDHSELSGINPYEARVKGLSQKL
SEEEFSAALLHLAKRRGVHNVNEVEEDTGNELSTKEQISRNSKALEEKYV
AELQLERLKKDGEVRGSINRFKTSDYVKEAKQLLKVQKAYHQLDQSFIDT
YIDLLETRRTYYEGPGEGSPFGWKDIKEWYEMLMGHCTYFPEELRSVKYA
YNADLYNALNDLNNLVITRDENEKLEYYEKFQIIENVFKQKKKPTLKQIA
KEILVNEEDIKGYRVTSTGKPEFTNLKVYHDIKDITARKEIIENAELLDQ
IAKILTIYQSSEDIQEELTNLNSELTQEEIEQISNLKGYTGTHNLSLKAI
NLILDELWHTNDNQIAIFNRLKLVPKKVDLSQQKEIPTTLVDDFILSPVV
KRSFIQSIKVINAIIKKYGLPNDIIIELAREKNSKDAQKMINEMQKRNRQ
TNERIEEIIRTTGKENAKYLIEKIKLHDMQEGKCLYSLEAIPLEDLLNNP
FNYEVDHIIPRSVSFDNSFNNKVLVKQEENSKKGNRTPFQYLSSSDSKIS
YETFKKHILNLAKGKGRISKTKKEYLLEERDINRFSVQKDFINRNLVDTR
YATRGLMNLLRSYFRVNNLDVKVKSINGGFTSFLRRKWKFKKERNKGYKH
HAEDALIIANADFIFKEWKKLDKAKKVMENQMFEEKQAESMPEIETEQEY
KEIFITPHQIKHIKDFKDYKYSHRVDKKPNRELINDTLYSTRKDDKGNTL
IVNNLNGLYDKDNDKLKKLINKSPEKLLMYHHDPQTYQKLKLIMEQYGDE
KNPLYKYYEETGNYLTKYSKKDNGPVIKKIKYYGNKLNAHLDITDDYPNS
RNKVVKLSLKPYRFDVYLDNGVYKFVTVKNLDVIKKENYYEVNSKCYEEA
KKLKKISNQAEFIASFYNNDLIKINGELYRVIGVNNDLLNRIEVNMIDIT
YREYLENMNDKRPPRIIKTIASKTQSIKKYSTDILGNLYEVKSKKHPQII
KKG.

Exemplary wild type Staphylococcus aureus Cas9 (SaCas9) amino acid sequence lacking N-terminus methionine:

(SEQ ID NO: 5800)
KRNYILGLDIGITSVGYGIIDYETRDVIDAGVRLFKEANVENNEGRRSKR
GARRLKRRRRHRIQRVKKLLFDYNLLTDHSELSGINPYEARVKGLSQKLS
EEEFSAALLHLAKRRGVHNVNEVEEDTGNELSTKEQISRNSKALEEKYVA
ELQLERLKKDGEVRGSINRFKTSDYVKEAKQLLKVQKAYHQLDQSFIDTY
IDLLETRRTYYEGPGEGSPFGWKDIKEWYEMLMGHCTYFPEELRSVKYAY
NADLYNALNDLNNLVITRDENEKLEYYEKFQIIENVFKQKKKPTLKQIAK
EILVNEEDIKGYRVTSTGKPEFTNLKVYHDIKDITARKEIIENAELLDQI
AKILTIYQSSEDIQEELTNLNSELTQEEIEQISNLKGYTGTHNLSLKAIN
LILDELWHTNDNQIAIFNRLKLVPKKVDLSQQKEIPTTLVDDFILSPVVK
RSFIQSIKVINAIIKKYGLPNDIIIELAREKNSKDAQKMINEMQKRNRQT
NERIEEIIRTTGKENAKYLIEKIKLHDMQEGKCLYSLEAIPLEDLLNNPF
NYEVDHIIPRSVSFDNSFNNKVLVKQEENSKKGNRTPFQYLSSSDSKISY
ETFKKHILNLAKGKGRISKTKKEYLLEERDINRFSVQKDFINRNLVDTRY
ATRGLMNLLRSYFRVNNLDVKVKSINGGFTSFLRRKWKFKKERNKGYKHH
AEDALIIANADFIFKEWKKLDKAKKVMENQMFEEKQAESMPEIETEQEYK
EIFITPHQIKHIKDFKDYKYSHRVDKKPNRELINDTLYSTRKDDKGNTLI
VNNLNGLYDKDNDKLKKLINKSPEKLLMYHHDPQTYQKLKLIMEQYGDEK
NPLYKYYEETGNYLTKYSKKDNGPVIKKIKYYGNKLNAHLDITDDYPNSR
NKVVKLSLKPYRFDVYLDNGVYKFVTVKNLDVIKKENYYEVNSKCYEEAK
KLKKISNQAEFIASFYNNDLIKINGELYRVIGVNNDLLNRIEVNMIDITY
REYLENMNDKRPPRIIKTIASKTQSIKKYSTDILGNLYEVKSKKHPQIIK
KG.

Exemplary Staphylococcus aureus Cas9 (SaCas9) nickase amino acid sequence:

(SEQ ID NO: 5802)
MKRNYILGLDIGITSVGYGIIDYETRDVIDAGVRLFKEANVENNEGRRSK
RGARRLKRRRRHRIQRVKKLLFDYNLLTDHSELSGINPYEARVKGLSQKL
SEEEFSAALLHLAKRRGVHNVNEVEEDTGNELSTKEQISRNSKALEEKYV
AELQLERLKKDGEVRGSINRFKTSDYVKEAKQLLKVQKAYHQLDQSFIDT
YIDLLETRRTYYEGPGEGSPFGWKDIKEWYEMLMGHCTYFPEELRSVKYA
YNADLYNALNDLNNLVITRDENEKLEYYEKFQIIENVFKQKKKPTLKQIA
KEILVNEEDIKGYRVTSTGKPEFTNLKVYHDIKDITARKEIIENAELLDQ
IAKILTIYQSSEDIQEELTNLNSELTQEEIEQISNLKGYTGTHNLSLKAI
NLILDELWHTNDNQIAIFNRLKLVPKKVDLSQQKEIPTTLVDDFILSPVV
KRSFIQSIKVINAIIKKYGLPNDIIIELAREKNSKDAQKMINEMQKRNRQ
TNERIEEIIRTTGKENAKYLIEKIKLHDMQEGKCLYSLEAIPLEDLLNNP
FNYEVDHIIPRSVSFDNSFNNKVLVKQEEASKKGNRTPFQYLSSSDSKIS
YETFKKHILNLAKGKGRISKTKKEYLLEERDINRFSVQKDFINRNLVDTR
YATRGLMNLLRSYFRVNNLDVKVKSINGGFTSFLRRKWKFKKERNKGYKH
HAEDALIIANADFIFKEWKKLDKAKKVMENQMFEEKQAESMPEIETEQEY
KEIFITPHQIKHIKDFKDYKYSHRVDKKPNRELINDTLYSTRKDDKGNTL
IVNNLNGLYDKDNDKLKKLINKSPEKLLMYHHDPQTYQKLKLIMEQYGDE
KNPLYKYYEETGNYLTKYSKKDNGPVIKKIKYYGNKLNAHLDITDDYPNS
RNKVVKLSLKPYRFDVYLDNGVYKFVTVKNLDVIKKENYYEVNSKCYEEA
KKLKKISNQAEFIASFYNNDLIKINGELYRVIGVNNDLLNRIEVNMIDIT
YREYLENMNDKRPPRIIKTIASKTQSIKKYSTDILGNLYEVKSKKHPQII
KKG.

Exemplary Staphylococcus aureus Cas9 (SaCas9) nickase amino acid sequence lacking N-terminus methionine:

(SEQ ID NO: 5803)
KRNYILGLDIGITSVGYGIIDYETRDVIDAGVRLFKEANVENNEGRRSKR
GARRLKRRRRHRIQRVKKLLFDYNLLTDHSELSGINPYEARVKGLSQKLS
EEEFSAALLHLAKRRGVHNVNEVEEDTGNELSTKEQISRNSKALEEKYVA
ELQLERLKKDGEVRGSINRFKTSDYVKEAKQLLKVQKAYHQLDQSFIDTY
IDLLETRRTYYEGPGEGSPFGWKDIKEWYEMLMGHCTYFPEELRSVKYAY
NADLYNALNDLNNLVITRDENEKLEYYEKFQIIENVFKQKKKPTLKQIAK
EILVNEEDIKGYRVTSTGKPEFTNLKVYHDIKDITARKEIIENAELLDQI
AKILTIYQSSEDIQEELTNLNSELTQEEIEQISNLKGYTGTHNLSLKAIN
LILDELWHTNDNQIAIFNRLKLVPKKVDLSQQKEIPTTLVDDFILSPVVK
RSFIQSIKVINAIIKKYGLPNDIIIELAREKNSKDAQKMINEMQKRNRQT
NERIEEIIRTTGKENAKYLIEKIKLHDMQEGKCLYSLEAIPLEDLLNNPF
NYEVDHIIPRSVSFDNSFNNKVLVKQEEASKKGNRTPFQYLSSSDSKISY
ETFKKHILNLAKGKGRISKTKKEYLLEERDINRFSVQKDFINRNLVDTRY
ATRGLMNLLRSYFRVNNLDVKVKSINGGFTSFLRRKWKFKKERNKGYKHH
AEDALIIANADFIFKEWKKLDKAKKVMENQMFEEKQAESMPEIETEQEYK
EIFITPHQIKHIKDFKDYKYSHRVDKKPNRELINDTLYSTRKDDKGNTLI
VNNLNGLYDKDNDKLKKLINKSPEKLLMYHHDPQTYQKLKLIMEQYGDEK
NPLYKYYEETGNYLTKYSKKDNGPVIKKIKYYGNKLNAHLDITDDYPNSR
NKVVKLSLKPYRFDVYLDNGVYKFVTVKNLDVIKKENYYEVNSKCYEEAK
KLKKISNQAEFIASFYNNDLIKINGELYRVIGVNNDLLNRIEVNMIDITY
REYLENMNDKRPPRIIKTIASKTQSIKKYSTDILGNLYEVKSKKHPQIIK
KG.

In some embodiments, a prime editor comprises a Cas protein, e.g., Cas9 variant, containing modifications that allow altered PAM recognition. Exemplary Cas9 variants with altered PAM specificities that are useful in the Prime editors of the disclosure are provided below in SEQ ID NOs 5804-5819.

Exemplary Streptococcus pyogenes Cas9 variant (SpRY) amino acid sequence:

(SEQ ID NO: 5804)
KRNYILGLDIGITSVGYGIIDYETRDVIDAGVRLFKEANVENNEGRRSKR
GARRLKRRRRHRIQRVKKLLFDYNLLTDHSELSGINPYEARVKGLSQKLS
EEEFSAALLHLAKRRGVHNVNEVEEDTGNELSTKEQISRNSKALEEKYVA
ELQLERLKKDGEVRGSINRFKTSDYVKEAKQLLKVQKAYHQLDQSFIDTY
IDLLETRRTYYEGPGEGSPFGWKDIKEWYEMLMGHCTYFPEELRSVKYAY
NADLYNALNDLNNLVITRDENEKLEYYEKFQIIENVFKQKKKPTLKQIAK
EILVNEEDIKGYRVTSTGKPEFTNLKVYHDIKDITARKEIIENAELLDQI
AKILTIYQSSEDIQEELTNLNSELTQEEIEQISNLKGYTGTHNLSLKAIN
LILDELWHTNDNQIAIFNRLKLVPKKVDLSQQKEIPTTLVDDFILSPVVK
RSFIQSIKVINAIIKKYGLPNDIIIELAREKNSKDAQKMINEMQKRNRQT
NERIEEIIRTTGKENAKYLIEKIKLHDMQEGKCLYSLEAIPLEDLLNNPF
NYEVDHIIPRSVSFDNSFNNKVLVKQEEASKKGNRTPFQYLSSSDSKISY
ETFKKHILNLAKGKGRISKTKKEYLLEERDINRFSVQKDFINRNLVDTRY
ATRGLMNLLRSYFRVNNLDVKVKSINGGFTSFLRRKWKFKKERNKGYKHH
AEDALIIANADFIFKEWKKLDKAKKVMENQMFEEKQAESMPEIETEQEYK
EIFITPHQIKHIKDFKDYKYSHRVDKKPNRELINDTLYSTRKDDKGNTLI
VNNLNGLYDKDNDKLKKLINKSPEKLLMYHHDPQTYQKLKLIMEQYGDEK
NPLYKYYEETGNYLTKYSKKDNGPVIKKIKYYGNKLNAHLDITDDYPNSR
NKVVKLSLKPYRFDVYLDNGVYKFVTVKNLDVIKKENYYEVNSKCYEEAK
KLKKISNQAEFIASFYNNDLIKINGELYRVIGVNNDLLNRIEVNMIDITY
REYLENMNDKRPPRIIKTIASKTQSIKKYSTDILGNLYEVKSKKHPQIIK
KG.

Exemplary Streptococcus pyogenes Cas9 variant (SpRY) amino acid sequence lacking N-terminus methionine:

(SEQ ID NO: 5805)
MDKKYSIGLDIGTNSVGWAVITDEYKVPSKKFKVLGNTDRHSIKKNLIGA
LLFDSGETAERTRLKRTARRRYTRRKNRICYLQEIFSNEMAKVDDSFFHR
LEESFLVEEDKKHERHPIFGNIVDEVAYHEKYPTIYHLRKKLVDSTDKAD
LRLIYLALAHMIKFRGHFLIEGDLNPDNSDVDKLFIQLVQTYNQLFEENP
INASGVDAKAILSARLSKSRRLENLIAQLPGEKKNGLFGNLIALSLGLTP
NFKSNFDLAEDAKLQLSKDTYDDDLDNLLAQIGDQYADLFLAAKNLSDAI
LLSDILRVNTEITKAPLSASMIKRYDEHHQDLTLLKALVRQQLPEKYKEI
FFDQSKNGYAGYIDGGASQEEFYKFIKPILEKMDGTEELLVKLNREDLLR
KQRTFDNGSIPHQIHLGELHAILRRQEDFYPFLKDNREKIEKILTFRIPY
YVGPLARGNSRFAWMTRKSEETITPWNFEEVVDKGASAQSFIERMTNFDK
NLPNEKVLPKHSLLYEYFTVYNELTKVKYVTEGMRKPAFLSGEQKKAIVD
LLFKTNRKVTVKQLKEDYFKKIECFDSVEISGVEDRFNASLGTYHDLLKI
IKDKDFLDNEENEDILEDIVLTLTLFEDREMIEERLKTYAHLFDDKVMKQ
LKRRRYTGWGRLSRKLINGIRDKQSGKTILDFLKSDGFANRNFMQLIHDD
SLTFKEDIQKAQVSGQGDSLHEHIANLAGSPAIKKGILQTVKVVDELVKV
MGRHKPENIVIEMARENQTTQKGQKNSRERMKRIEEGIKELGSQILKEHP
VENTQLQNEKLYLYYLQNGRDMYVDQELDINRLSDYDVDHIVPQSFLKDD
SIDNKVLTRSDKNRGKSDNVPSEEVVKKMKNYWRQLLNAKLITQRKFDNL
TKAERGGLSELDKAGFIKRQLVETRQITKHVAQILDSRMNTKYDENDKLI
REVKVITLKSKLVSDFRKDFQFYKVREINNYHHAHDAYLNAVVGTALIKK
YPKLESEFVYGDYKVYDVRKMIAKSEQEIGKATAKYFFYSNIMNFFKTEI
TLANGEIRKRPLIETNGETGEIVWDKGRDFATVRKVLSMPQVNIVKKTEV
QTGGFSKESIRPKRNSDKLIARKKDWDPKKYGGFLWPTVAYSVLVVAKVE
KGKSKKLKSVKELLGITIMERSSFEKNPIDFLEAKGYKEVKKDLIIKLPK
YSLFELENGRKRMLASAKQLQKGNELALPSKYVNFLYLASHYEKLKGSPE
DNEQKQLFVEQHKHYLDEIIEQISEFSKRVILADANLDKVLSAYNKHRDK
PIREQAENIIHLFTLTRLGAPRAFKYFDTTIDPKQYRSTKEVLDATLIHQ
SITGLYETRIDLSQLGGD.

Exemplary Streptococcus pyogenes Cas9 variant nickase (SpRY nickase) amino acid sequence:

(SEQ ID NO: 5806)
MDKKYSIGLDIGTNSVGWAVITDEYKVPSKKFKVLGNTDRHSIKKNLIGA
LLFDSGETAERTRLKRTARRRYTRRKNRICYLQEIFSNEMAKVDDSFFHR
LEESFLVEEDKKHERHPIFGNIVDEVAYHEKYPTIYHLRKKLVDSTDKAD
LRLIYLALAHMIKFRGHFLIEGDLNPDNSDVDKLFIQLVQTYNQLFEENP
INASGVDAKAILSARLSKSRRLENLIAQLPGEKKNGLFGNLIALSLGLTP
NFKSNFDLAEDAKLQLSKDTYDDDLDNLLAQIGDQYADLFLAAKNLSDAI
LLSDILRVNTEITKAPLSASMIKRYDEHHQDLTLLKALVRQQLPEKYKEI
FFDQSKNGYAGYIDGGASQEEFYKFIKPILEKMDGTEELLVKLNREDLLR
KQRTFDNGSIPHQIHLGELHAILRRQEDFYPFLKDNREKIEKILTFRIPY
YVGPLARGNSRFAWMTRKSEETITPWNFEEVVDKGASAQSFIERMTNFDK
NLPNEKVLPKHSLLYEYFTVYNELTKVKYVTEGMRKPAFLSGEQKKAIVD
LLFKTNRKVTVKQLKEDYFKKIECFDSVEISGVEDRFNASLGTYHDLLKI
IKDKDFLDNEENEDILEDIVLTLTLFEDREMIEERLKTYAHLFDDKVMKQ
LKRRRYTGWGRLSRKLINGIRDKQSGKTILDFLKSDGFANRNFMQLIHDD
SLTFKEDIQKAQVSGQGDSLHEHIANLAGSPAIKKGILQTVKVVDELVKV
MGRHKPENIVIEMARENQTTQKGQKNSRERMKRIEEGIKELGSQILKEHP
VENTQLQNEKLYLYYLQNGRDMYVDQELDINRLSDYDVDAIVPQSFLKDD
SIDNKVLTRSDKNRGKSDNVPSEEVVKKMKNYWRQLLNAKLITQRKFDNL
TKAERGGLSELDKAGFIKRQLVETRQITKHVAQILDSRMNTKYDENDKLI
REVKVITLKSKLVSDFRKDFQFYKVREINNYHHAHDAYLNAVVGTALIKK
YPKLESEFVYGDYKVYDVRKMIAKSEQEIGKATAKYFFYSNIMNFFKTEI
TLANGEIRKRPLIETNGETGEIVWDKGRDFATVRKVLSMPQVNIVKKTEV
QTGGFSKESIRPKRNSDKLIARKKDWDPKKYGGFLWPTVAYSVLVVAKVE
KGKSKKLKSVKELLGITIMERSSFEKNPIDFLEAKGYKEVKKDLIIKLPK
YSLFELENGRKRMLASAKQLQKGNELALPSKYVNFLYLASHYEKLKGSPE
DNEQKQLFVEQHKHYLDEIIEQISEFSKRVILADANLDKVLSAYNKHRDK
PIREQAENIIHLFTLTRLGAPRAFKYFDTTIDPKQYRSTKEVLDATLIHQ
SITGLYETRIDLSQLGGD.

Exemplary Streptococcus pyogenes Cas9 variant nickase (SpRY nickase) amino acid sequence lacking N-terminus methionine:

(SEQ ID NO: 5807)
DKKYSIGLDIGTNSVGWAVITDEYKVPSKKFKVLGNTDRHSIKKNLIGAL
LFDSGETAERTRLKRTARRRYTRRKNRICYLQEIFSNEMAKVDDSFFHRL
EESFLVEEDKKHERHPIFGNIVDEVAYHEKYPTIYHLRKKLVDSTDKADL
RLIYLALAHMIKFRGHFLIEGDLNPDNSDVDKLFIQLVQTYNQLFEENPI
NASGVDAKAILSARLSKSRRLENLIAQLPGEKKNGLFGNLIALSLGLTPN
FKSNFDLAEDAKLQLSKDTYDDDLDNLLAQIGDQYADLFLAAKNLSDAIL
LSDILRVNTEITKAPLSASMIKRYDEHHQDLTLLKALVRQQLPEKYKEIF
FDQSKNGYAGYIDGGASQEEFYKFIKPILEKMDGTEELLVKLNREDLLRK
QRTFDNGSIPHQIHLGELHAILRRQEDFYPFLKDNREKIEKILTFRIPYY
VGPLARGNSRFAWMTRKSEETITPWNFEEVVDKGASAQSFIERMTNFDKN
LPNEKVLPKHSLLYEYFTVYNELTKVKYVTEGMRKPAFLSGEQKKAIVDL
LFKTNRKVTVKQLKEDYFKKIECFDSVEISGVEDRFNASLGTYHDLLKII
KDKDFLDNEENEDILEDIVLTLTLFEDREMIEERLKTYAHLFDDKVMKQL
KRRRYTGWGRLSRKLINGIRDKQSGKTILDFLKSDGFANRNFMQLIHDDS
LTFKEDIQKAQVSGQGDSLHEHIANLAGSPAIKKGILQTVKVVDELVKVM
GRHKPENIVIEMARENQTTQKGQKNSRERMKRIEEGIKELGSQILKEHPV
ENTQLQNEKLYLYYLQNGRDMYVDQELDINRLSDYDVDAIVPQSFLKDDS
IDNKVLTRSDKNRGKSDNVPSEEVVKKMKNYWRQLLNAKLITQRKFDNLT
KAERGGLSELDKAGFIKRQLVETRQITKHVAQILDSRMNTKYDENDKLIR
EVKVITLKSKLVSDFRKDFQFYKVREINNYHHAHDAYLNAVVGTALIKKY
PKLESEFVYGDYKVYDVRKMIAKSEQEIGKATAKYFFYSNIMNFFKTEIT
LANGEIRKRPLIETNGETGEIVWDKGRDFATVRKVLSMPQVNIVKKTEVQ
TGGFSKESIRPKRNSDKLIARKKDWDPKKYGGFLWPTVAYSVLVVAKVEK
GKSKKLKSVKELLGITIMERSSFEKNPIDFLEAKGYKEVKKDLIIKLPKY
SLFELENGRKRMLASAKQLQKGNELALPSKYVNFLYLASHYEKLKGSPED
NEQKQLFVEQHKHYLDEIIEQISEFSKRVILADANLDKVLSAYNKHRDKP
IREQAENIIHLFTLTRLGAPRAFKYFDTTIDPKQYRSTKEVLDATLIHQS
ITGLYETRIDLSQLGGD.

Exemplary Streptococcus pyogenes Cas9 variant (SpG) amino acid sequence:

(SEQ ID NO: 5816)
MDKKYSIGLDIGTNSVGWAVITDEYKVPSKKFKVLGNTDRHSIKKNLIGA
LLFDSGETAEATRLKRTARRRYTRRKNRICYLQEIFSNEMAKVDDSFFHR
LEESFLVEEDKKHERHPIFGNIVDEVAYHEKYPTIYHLRKKLVDSTDKAD
LRLIYLALAHMIKFRGHFLIEGDLNPDNSDVDKLFIQLVQTYNQLFEENP
INASGVDAKAILSARLSKSRRLENLIAQLPGEKKNGLFGNLIALSLGLTP
NFKSNFDLAEDAKLQLSKDTYDDDLDNLLAQIGDQYADLFLAAKNLSDAI
LLSDILRVNTEITKAPLSASMIKRYDEHHQDLTLLKALVRQQLPEKYKEI
FFDQSKNGYAGYIDGGASQEEFYKFIKPILEKMDGTEELLVKLNREDLLR
KQRTFDNGSIPHQIHLGELHAILRRQEDFYPFLKDNREKIEKILTFRIPY
YVGPLARGNSRFAWMTRKSEETITPWNFEEVVDKGASAQSFIERMTNFDK
NLPNEKVLPKHSLLYEYFTVYNELTKVKYVTEGMRKPAFLSGEQKKAIVD
LLFKTNRKVTVKQLKEDYFKKIECFDSVEISGVEDRFNASLGTYHDLLKI
IKDKDFLDNEENEDILEDIVLTLTLFEDREMIEERLKTYAHLFDDKVMKQ
LKRRRYTGWGRLSRKLINGIRDKQSGKTILDFLKSDGFANRNFMQLIHDD
SLTFKEDIQKAQVSGQGDSLHEHIANLAGSPAIKKGILQTVKVVDELVKV
MGRHKPENIVIEMARENQTTQKGQKNSRERMKRIEEGIKELGSQILKEHP
VENTQLQNEKLYLYYLQNGRDMYVDQELDINRLSDYDVDHIVPQSFLKDD
SIDNKVLTRSDKNRGKSDNVPSEEVVKKMKNYWRQLLNAKLITQRKFDNL
TKAERGGLSELDKAGFIKRQLVETRQITKHVAQILDSRMNTKYDENDKLI
REVKVITLKSKLVSDFRKDFQFYKVREINNYHHAHDAYLNAVVGTALIKK
YPKLESEFVYGDYKVYDVRKMIAKSEQEIGKATAKYFFYSNIMNFFKTEI
TLANGEIRKRPLIETNGETGEIVWDKGRDFATVRKVLSMPQVNIVKKTEV
QTGGFSKESILPKRNSDKLIARKKDWDPKKYGGFLWPTVAYSVLVVAKVE
KGKSKKLKSVKELLGITIMERSSFEKNPIDFLEAKGYKEVKKDLIIKLPK
YSLFELENGRKRMLASAKQLQKGNELALPSKYVNFLYLASHYEKLKGSPE
DNEQKQLFVEQHKHYLDEIIEQISEFSKRVILADANLDKVLSAYNKHRDK
PIREQAENIIHLFTLTNLGAPAAFKYFDTTIDRKQYRSTKEVLDATLIHQ
SITGLYETRIDLSQLGGD.

Exemplary Streptococcus pyogenes Cas9 variant (SpG) amino acid sequence lacking N-terminus methionine:

(SEQ ID NO: 5817)
DKKYSIGLDIGTNSVGWAVITDEYKVPSKKFKVLGNTDRHSIKKNLIGAL
LFDSGETAEATRLKRTARRRYTRRKNRICYLQEIFSNEMAKVDDSFFHRL
EESFLVEEDKKHERHPIFGNIVDEVAYHEKYPTIYHLRKKLVDSTDKADL
RLIYLALAHMIKFRGHFLIEGDLNPDNSDVDKLFIQLVQTYNQLFEENPI
NASGVDAKAILSARLSKSRRLENLIAQLPGEKKNGLFGNLIALSLGLTPN
FKSNFDLAEDAKLQLSKDTYDDDLDNLLAQIGDQYADLFLAAKNLSDAIL
LSDILRVNTEITKAPLSASMIKRYDEHHQDLTLLKALVRQQLPEKYKEIF
FDQSKNGYAGYIDGGASQEEFYKFIKPILEKMDGTEELLVKLNREDLLRK
QRTFDNGSIPHQIHLGELHAILRRQEDFYPFLKDNREKIEKILTFRIPYY
VGPLARGNSRFAWMTRKSEETITPWNFEEVVDKGASAQSFIERMTNFDKN
LPNEKVLPKHSLLYEYFTVYNELTKVKYVTEGMRKPAFLSGEQKKAIVDL
LFKTNRKVTVKQLKEDYFKKIECFDSVEISGVEDRFNASLGTYHDLLKII
KDKDFLDNEENEDILEDIVLTLTLFEDREMIEERLKTYAHLFDDKVMKQL
KRRRYTGWGRLSRKLINGIRDKQSGKTILDFLKSDGFANRNFMQLIHDDS
LTFKEDIQKAQVSGQGDSLHEHIANLAGSPAIKKGILQTVKVVDELVKVM
GRHKPENIVIEMARENQTTQKGQKNSRERMKRIEEGIKELGSQILKEHPV
ENTQLQNEKLYLYYLQNGRDMYVDQELDINRLSDYDVDHIVPQSFLKDDS
IDNKVLTRSDKNRGKSDNVPSEEVVKKMKNYWRQLLNAKLITQRKFDNLT
KAERGGLSELDKAGFIKRQLVETRQITKHVAQILDSRMNTKYDENDKLIR
EVKVITLKSKLVSDFRKDFQFYKVREINNYHHAHDAYLNAVVGTALIKKY
PKLESEFVYGDYKVYDVRKMIAKSEQEIGKATAKYFFYSNIMNFFKTEIT
LANGEIRKRPLIETNGETGEIVWDKGRDFATVRKVLSMPQVNIVKKTEVQ
TGGFSKESILPKRNSDKLIARKKDWDPKKYGGFLWPTVAYSVLVVAKVEK
GKSKKLKSVKELLGITIMERSSFEKNPIDFLEAKGYKEVKKDLIIKLPKY
SLFELENGRKRMLASAKQLQKGNELALPSKYVNFLYLASHYEKLKGSPED
NEQKQLFVEQHKHYLDEIIEQISEFSKRVILADANLDKVLSAYNKHRDKP
IREQAENIIHLFTLTNLGAPAAFKYFDTTIDRKQYRSTKEVLDATLIHQS
ITGLYETRIDLSQLGGD.

Exemplary Streptococcus pyogenes Cas9 variant (SpG nickase) amino acid sequence:

(SEQ ID NO: 5818)
MDKKYSIGLDIGTNSVGWAVITDEYKVPSKKFKVLGNTDRHSIKKNLIGA
LLFDSGETAEATRLKRTARRRYTRRKNRICYLQEIFSNEMAKVDDSFFHR
LEESFLVEEDKKHERHPIFGNIVDEVAYHEKYPTIYHLRKKLVDSTDKAD
LRLIYLALAHMIKFRGHFLIEGDLNPDNSDVDKLFIQLVQTYNQLFEENP
INASGVDAKAILSARLSKSRRLENLIAQLPGEKKNGLFGNLIALSLGLTP
NFKSNFDLAEDAKLQLSKDTYDDDLDNLLAQIGDQYADLFLAAKNLSDAI
LLSDILRVNTEITKAPLSASMIKRYDEHHQDLTLLKALVRQQLPEKYKEI
FFDQSKNGYAGYIDGGASQEEFYKFIKPILEKMDGTEELLVKLNREDLLR
KQRTFDNGSIPHQIHLGELHAILRRQEDFYPFLKDNREKIEKILTFRIPY
YVGPLARGNSRFAWMTRKSEETITPWNFEEVVDKGASAQSFIERMTNFDK
NLPNEKVLPKHSLLYEYFTVYNELTKVKYVTEGMRKPAFLSGEQKKAIVD
LLFKTNRKVTVKQLKEDYFKKIECFDSVEISGVEDRFNASLGTYHDLLKI
IKDKDFLDNEENEDILEDIVLTLTLFEDREMIEERLKTYAHLFDDKVMKQ
LKRRRYTGWGRLSRKLINGIRDKQSGKTILDFLKSDGFANRNFMQLIHDD
SLTFKEDIQKAQVSGQGDSLHEHIANLAGSPAIKKGILQTVKVVDELVKV
MGRHKPENIVIEMARENQTTQKGQKNSRERMKRIEEGIKELGSQILKEHP
VENTQLQNEKLYLYYLQNGRDMYVDQELDINRLSDYDVDAIVPQSFLKDD
SIDNKVLTRSDKNRGKSDNVPSEEVVKKMKNYWRQLLNAKLITQRKFDNL
TKAERGGLSELDKAGFIKRQLVETRQITKHVAQILDSRMNTKYDENDKLI
REVKVITLKSKLVSDFRKDFQFYKVREINNYHHAHDAYLNAVVGTALIKK
YPKLESEFVYGDYKVYDVRKMIAKSEQEIGKATAKYFFYSNIMNFFKTEI
TLANGEIRKRPLIETNGETGEIVWDKGRDFATVRKVLSMPQVNIVKKTEV
QTGGFSKESILPKRNSDKLIARKKDWDPKKYGGFLWPTVAYSVLVVAKVE
KGKSKKLKSVKELLGITIMERSSFEKNPIDFLEAKGYKEVKKDLIIKLPK
YSLFELENGRKRMLASAKQLQKGNELALPSKYVNFLYLASHYEKLKGSPE
DNEQKQLFVEQHKHYLDEIIEQISEFSKRVILADANLDKVLSAYNKHRDK
PIREQAENIIHLFTLTNLGAPAAFKYFDTTIDRKQYRSTKEVLDATLIHQ
SITGLYETRIDLSQLGGD.

Exemplary Streptococcus pyogenes Cas9 variant (SpG nickase) amino acid sequence lacking N-terminus methionine:

(SEQ ID NO: 5819)
DKKYSIGLDIGTNSVGWAVITDEYKVPSKKFKVLGNTDRHSIKKNLIGAL
LFDSGETAEATRLKRTARRRYTRRKNRICYLQEIFSNEMAKVDDSFFHRL
EESFLVEEDKKHERHPIFGNIVDEVAYHEKYPTIYHLRKKLVDSTDKADL
RLIYLALAHMIKFRGHFLIEGDLNPDNSDVDKLFIQLVQTYNQLFEENPI
NASGVDAKAILSARLSKSRRLENLIAQLPGEKKNGLFGNLIALSLGLTPN
FKSNFDLAEDAKLQLSKDTYDDDLDNLLAQIGDQYADLFLAAKNLSDAIL
LSDILRVNTEITKAPLSASMIKRYDEHHQDLTLLKALVRQQLPEKYKEIF
FDQSKNGYAGYIDGGASQEEFYKFIKPILEKMDGTEELLVKLNREDLLRK
QRTFDNGSIPHQIHLGELHAILRRQEDFYPFLKDNREKIEKILTFRIPYY
VGPLARGNSRFAWMTRKSEETITPWNFEEVVDKGASAQSFIERMTNFDKN
LPNEKVLPKHSLLYEYFTVYNELTKVKYVTEGMRKPAFLSGEQKKAIVDL
LFKTNRKVTVKQLKEDYFKKIECFDSVEISGVEDRFNASLGTYHDLLKII
KDKDFLDNEENEDILEDIVLTLTLFEDREMIEERLKTYAHLFDDKVMKQL
KRRRYTGWGRLSRKLINGIRDKQSGKTILDFLKSDGFANRNFMQLIHDDS
LTFKEDIQKAQVSGQGDSLHEHIANLAGSPAIKKGILQTVKVVDELVKVM
GRHKPENIVIEMARENQTTQKGQKNSRERMKRIEEGIKELGSQILKEHPV
ENTQLQNEKLYLYYLQNGRDMYVDQELDINRLSDYDVDAIVPQSFLKDDS
IDNKVLTRSDKNRGKSDNVPSEEVVKKMKNYWRQLLNAKLITQRKFDNLT
KAERGGLSELDKAGFIKRQLVETRQITKHVAQILDSRMNTKYDENDKLIR
EVKVITLKSKLVSDFRKDFQFYKVREINNYHHAHDAYLNAVVGTALIKKY
PKLESEFVYGDYKVYDVRKMIAKSEQEIGKATAKYFFYSNIMNFFKTEIT
LANGEIRKRPLIETNGETGEIVWDKGRDFATVRKVLSMPQVNIVKKTEVQ
TGGFSKESILPKRNSDKLIARKKDWDPKKYGGFLWPTVAYSVLVVAKVEK
GKSKKLKSVKELLGITIMERSSFEKNPIDFLEAKGYKEVKKDLIIKLPKY
SLFELENGRKRMLASAKQLQKGNELALPSKYVNFLYLASHYEKLKGSPED
NEQKQLFVEQHKHYLDEIIEQISEFSKRVILADANLDKVLSAYNKHRDKP
IREQAENIIHLFTLTNLGAPAAFKYFDTTIDRKQYRSTKEVLDATLIHQS
ITGLYETRIDLSQLGGD.

In some embodiments, a Cas9 is a chimeric Cas9, e.g., modified Cas9; e.g., synthetic RNA-guided nucleases (sRGNs), e.g., modified by DNA family shuffling, e.g., sRGN3.1, sRGN3.3. In some embodiments, the DNA family shuffling comprises, fragmentation and reassembly of parental Cas9 genes, e.g., one or more of Cas9s from Staphylococcus hyicus (Shy), Staphylococcus lugdunensis (Slu), Staphylococcus microti (Smi), and Staphylococcus pasteuri (Spa). In some embodiments, a modified sluCas9 shows increased editing efficiency and/or specificity relative to a sluCas9 that is not modified. In some embodiments, a modified Cas9, e.g., a sRGN shows at least 10%, at least 20%, at least 30%, at least 40%, at least 50%, at least 60%, at least 70%, at least 80%, at least 90%, at least 100%, at least 150%, at least 200%, at least 300%, at least 400%, at least 500%, at least 600%, at least 700%, at least 800%, at least 900%, or at least 1000% increase in editing efficiency compared to a Cas9 that is not modified. In some embodiments, a Cas9, e.g., a sRGN shows at least 10%, at least 20%, at least 30%, at least 40%, at least 50%, at least 60%, at least 70%, at least 80%, at least 90%, at least 100%, at least 150%, at least 200%, at least 300%, at least 400%, at least 500%, at least 600%, at least 700%, at least 800%, at least 900%, or at least 1000% increase in specificity compared to a Cas9 that is not modified. In some embodiments, a Cas9, e.g., a sRGN shows at least 10%, at least 20%, at least 30%, at least 40%, at least 50%, at least 60%, at least 70%, at least 80%, at least 90%, at least 100%, at least 150%, at least 200%, at least 300%, at least 400%, at least 500%, at least 600%, at least 700%, at least 800%, at least 900%, or at least 1000% increase in cleavage activity compared to a Cas9 that is not modified. In some embodiments, a Cas9, e.g., a sRGN shows ability to cleave a 5′-NNGG-3′ PAM-containing target. In some embodiments, a prime editor may comprise a Cas9 (e.g., a chimeric Cas9), e.g., as according any of the sequences selected from 5808-5815 or a variant thereof. Exemplary amino acid sequences of sRGN useful in the prime editors disclosed herein are provided below in SEQ ID NOs: 5808-5815.

Exemplary sRGN3.1 amino acid sequence:

(SEQ ID NO: 5808)
MNQKFILGLDIGITSVGYGLIDYETKNIIDAGVRLFPEANVENNEGRRSK
RGSRRLKRRRIHRLERVKLLLTEYDLINKEQIPTSNNPYQIRVKGLSEIL
SKDELAIALLHLAKRRGIHNVDVAADKEETASDSLSTKDQINKNAKFLES
RYVCELQKERLENEGHVRGVENRFLTKDIVREAKKIIDTQMQYYPEIDET
FKEKYISLVETRREYFEGPGQGSPFGWNGDLKKWYEMLMGHCTYFPQELR
SVKYAYSADLFNALNDLNNLIIQRDNSEKLEYHEKYHIIENVFKQKKKPT
LKQIAKEIGVNPEDIKGYRITKSGTPEFTSFKLFHDLKKVVKDHAILDDI
DLLNQIAEILTIYQDKDSIVAELGQLEYLMSEADKQSISELTGYTGTHSL
SLKCMNMIIDELWHSSMNQMEVFTYLNMRPKKYELKGYQRIPTDMIDDAI
LSPVVKRTFIQSINVINKVIEKYGIPEDIIIELARENNSDDRKKFINNLQ
KKNEATRKRINEIIGQTGNQNAKRIVEKIRLHDQQEGKCLYSLESIPLED
LLNNPNHYEVDHIIPRSVSFDNSYHNKVLVKQSENSKKSNLTPYQYFNSG
KSKLSYNQFKQHILNLSKSQDRISKKKKEYLLEERDINKFEVQKEFINRN
LVDTRYATRELTNYLKAYFSANNMNVKVKTINGSFTDYLRKVWKFKKERN
HGYKHHAEDALIIANADFLFKENKKLKAVNSVLEKPEIETKQLDIQVDSE
DNYSEMFIIPKQVQDIKDFRNFKYSHRVDKKPNRQLINDTLYSTRKKDNS
TYIVQTIKDIYAKDNTTLKKQFDKSPEKFLMYQHDPRTFEKLEVIMKQYA
NEKNPLAKYHEETGEYLTKYSKKNNGPIVKSLKYIGNKLGSHLDVTHQFK
SSTKKLVKLSIKNYRFDVYLTEKGYKFVTIAYLNVFKKDNYYYIPKDKYQ
ELKEKKKIKDTDQFIASFYKNDLIKLNGDLYKIIGVNSDDRNIIELDYYD
IKYKDYCEINNIKGEPRIKKTIGKKTESIEKFTTDVLGNLYLHSTEKAPQ
LIFKRGL.

Exemplary sRGN3.1 amino acid sequence lacking N-terminus methionine:

(SEQ ID NO: 5809)
NQKFILGLDIGITSVGYGLIDYETKNIIDAGVRLFPEANVENNEGRRSKR
GSRRLKRRRIHRLERVKLLLTEYDLINKEQIPTSNNPYQIRVKGLSEILS
KDELAIALLHLAKRRGIHNVDVAADKEETASDSLSTKDQINKNAKFLESR
YVCELQKERLENEGHVRGVENRFLTKDIVREAKKIIDTQMQYYPEIDETF
KEKYISLVETRREYFEGPGQGSPFGWNGDLKKWYEMLMGHCTYFPQELRS
VKYAYSADLFNALNDLNNLIIQRDNSEKLEYHEKYHIIENVFKQKKKPTL
KQIAKEIGVNPEDIKGYRITKSGTPEFTSFKLFHDLKKVVKDHAILDDID
LLNQIAEILTIYQDKDSIVAELGQLEYLMSEADKQSISELTGYTGTHSLS
LKCMNMIIDELWHSSMNQMEVFTYLNMRPKKYELKGYQRIPTDMIDDAIL
SPVVKRTFIQSINVINKVIEKYGIPEDIIIELARENNSDDRKKFINNLQK
KNEATRKRINEIIGQTGNQNAKRIVEKIRLHDQQEGKCLYSLESIPLEDL
LNNPNHYEVDHIIPRSVSFDNSYHNKVLVKQSENSKKSNLTPYQYFNSGK
SKLSYNQFKQHILNLSKSQDRISKKKKEYLLEERDINKFEVQKEFINRNL
VDTRYATRELTNYLKAYFSANNMNVKVKTINGSFTDYLRKVWKFKKERNH
GYKHHAEDALIIANADFLFKENKKLKAVNSVLEKPEIETKQLDIQVDSED
NYSEMFIIPKQVQDIKDFRNFKYSHRVDKKPNRQLINDTLYSTRKKDNST
YIVQTIKDIYAKDNTTLKKQFDKSPEKFLMYQHDPRTFEKLEVIMKQYAN
EKNPLAKYHEETGEYLTKYSKKNNGPIVKSLKYIGNKLGSHLDVTHQFKS
STKKLVKLSIKNYRFDVYLTEKGYKFVTIAYLNVFKKDNYYYIPKDKYQE
LKEKKKIKDTDQFIASFYKNDLIKLNGDLYKIIGVNSDDRNIIELDYYDI
KYKDYCEINNIKGEPRIKKTIGKKTESIEKFTTDVLGNLYLHSTEKAPQL
IFKRGL.

Exemplary sRGN3.1 nickase amino acid sequence:

(SEQ ID NO: 5810)
MNQKFILGLDIGITSVGYGLIDYETKNIIDAGVRLFPEANVENNEGRRSK
RGSRRLKRRRIHRLERVKLLLTEYDLINKEQIPTSNNPYQIRVKGLSEIL
SKDELAIALLHLAKRRGIHNVDVAADKEETASDSLSTKDQINKNAKFLES
RYVCELQKERLENEGHVRGVENRFLTKDIVREAKKIIDTQMQYYPEIDET
FKEKYISLVETRREYFEGPGQGSPFGWNGDLKKWYEMLMGHCTYFPQELR
SVKYAYSADLFNALNDLNNLIIQRDNSEKLEYHEKYHIIENVFKQKKKPT
LKQIAKEIGVNPEDIKGYRITKSGTPEFTSFKLFHDLKKVVKDHAILDDI
DLLNQIAEILTIYQDKDSIVAELGQLEYLMSEADKQSISELTGYTGTHSL
SLKCMNMIIDELWHSSMNQMEVFTYLNMRPKKYELKGYQRIPTDMIDDAI
LSPVVKRTFIQSINVINKVIEKYGIPEDIIIELARENNSDDRKKFINNLQ
KKNEATRKRINEIIGQTGNQNAKRIVEKIRLHDQQEGKCLYSLESIPLED
LLNNPNHYEVDHIIPRSVSFDNSYHNKVLVKQSEASKKSNLTPYQYFNSG
KSKLSYNQFKQHILNLSKSQDRISKKKKEYLLEERDINKFEVQKEFINRN
LVDTRYATRELTNYLKAYFSANNMNVKVKTINGSFTDYLRKVWKFKKERN
HGYKHHAEDALIIANADFLFKENKKLKAVNSVLEKPEIETKQLDIQVDSE
DNYSEMFIIPKQVQDIKDFRNFKYSHRVDKKPNRQLINDTLYSTRKKDNS
TYIVQTIKDIYAKDNTTLKKQFDKSPEKFLMYQHDPRTFEKLEVIMKQYA
NEKNPLAKYHEETGEYLTKYSKKNNGPIVKSLKYIGNKLGSHLDVTHQFK
SSTKKLVKLSIKNYRFDVYLTEKGYKFVTIAYLNVFKKDNYYYIPKDKYQ
ELKEKKKIKDTDQFIASFYKNDLIKLNGDLYKIIGVNSDDRNIIELDYYD
IKYKDYCEINNIKGEPRIKKTIGKKTESIEKFTTDVLGNLYLHSTEKAPQ
LIFKRGL.

Exemplary sRGN3.1 nickase amino acid sequence lacking N-terminus methionine:

(SEQ ID NO: 5811)
NQKFILGLDIGITSVGYGLIDYETKNIIDAGVRLFPEANVENNEGRRSKR
GSRRLKRRRIHRLERVKLLLTEYDLINKEQIPTSNNPYQIRVKGLSEILS
KDELAIALLHLAKRRGIHNVDVAADKEETASDSLSTKDQINKNAKFLESR
YVCELQKERLENEGHVRGVENRFLTKDIVREAKKIIDTQMQYYPEIDETF
KEKYISLVETRREYFEGPGQGSPFGWNGDLKKWYEMLMGHCTYFPQELRS
VKYAYSADLFNALNDLNNLIIQRDNSEKLEYHEKYHIIENVFKQKKKPTL
KQIAKEIGVNPEDIKGYRITKSGTPEFTSFKLFHDLKKVVKDHAILDDID
LLNQIAEILTIYQDKDSIVAELGQLEYLMSEADKQSISELTGYTGTHSLS
LKCMNMIIDELWHSSMNQMEVFTYLNMRPKKYELKGYQRIPTDMIDDAIL
SPVVKRTFIQSINVINKVIEKYGIPEDIIIELARENNSDDRKKFINNLQK
KNEATRKRINEIIGQTGNQNAKRIVEKIRLHDQQEGKCLYSLESIPLEDL
LNNPNHYEVDHIIPRSVSFDNSYHNKVLVKQSEASKKSNLTPYQYFNSGK
SKLSYNQFKQHILNLSKSQDRISKKKKEYLLEERDINKFEVQKEFINRNL
VDTRYATRELTNYLKAYFSANNMNVKVKTINGSFTDYLRKVWKFKKERNH
GYKHHAEDALIIANADFLFKENKKLKAVNSVLEKPEIETKQLDIQVDSED
NYSEMFIIPKQVQDIKDFRNFKYSHRVDKKPNRQLINDTLYSTRKKDNST
YIVQTIKDIYAKDNTTLKKQFDKSPEKFLMYQHDPRTFEKLEVIMKQYAN
EKNPLAKYHEETGEYLTKYSKKNNGPIVKSLKYIGNKLGSHLDVTHQFKS
STKKLVKLSIKNYRFDVYLTEKGYKFVTIAYLNVFKKDNYYYIPKDKYQE
LKEKKKIKDTDQFIASFYKNDLIKLNGDLYKIIGVNSDDRNIIELDYYDI
KYKDYCEINNIKGEPRIKKTIGKKTESIEKFTTDVLGNLYLHSTEKAPQL
IFKRGL.

Exemplary sRGN3.3 amino acid sequence:

(SEQ ID NO: 5812)
MNQKFILGLDIGITSVGYGLIDYETKNIIDAGVRLFPEANVENNEGRRSK
RGSRRLKRRRIHRLERVKLLLTEYDLINKEQIPTSNNPYQIRVKGLSEIL
SKDELAIALLHLAKRRGIHNVDVAADKEETASDSLSTKDQINKNAKFLES
RYVCELQKERLENEGHVRGVENRFLTKDIVREAKKIIDTQMQYYPEIDET
FKEKYISLVETRREYFEGPGQGSPFGWNGDLKKWYEMLMGHCTYFPQELR
SVKYAYSADLFNALNDLNNLIIQRDNSEKLEYHEKYHIIENVFKQKKKPT
LKQIAKEIGVNPEDIKGYRITKSGTPEFTSFKLFHDLKKVVKDHAILDDI
DLLNQIAEILTIYQDKDSIVAELGQLEYLMSEADKQSISELTGYTGTHSL
SLKCMNMIIDELWHSSMNQMEVFTYLNMRPKKYELKGYQRIPTDMIDDAI
LSPVVKRTFIQSINVINKVIEKYGIPEDIIIELARENNSDDRKKFINNLQ
KKNEATRKRINEIIGQTGNQNAKRIVEKIRLHDQQEGKCLYSLESIPLED
LLNNPNHYEVDHIIPRSVSFDNSYHNKVLVKQSENSKKSNLTPYQYFNSG
KSKLSYNQFKQHILNLSKSQDRISKKKKEYLLEERDINKFEVQKEFINRN
LVDTRYATRELTSYLKAYFSANNMDVKVKTINGSFTNHLRKVWRFDKYRN
HGYKHHAEDALIIANADFLFKENKKLQNTNKILEKPTIENNTKKVTVEKE
EDYNNVFETPKLVEDIKQYRDYKFSHRVDKKPNRQLINDTLYSTRMKDEH
DYIVQTITDIYGKDNTNLKKQFNKNPEKFLMYQNDPKTFEKLSIIMKQYS
DEKNPLAKYYEETGEYLTKYSKKNNGPIVKKIKLLGNKVGNHLDVTNKYE
NSTKKLVKLSIKNYRFDVYLTEKGYKFVTIAYLNVFKKDNYYYIPKDKYQ
ELKEKKKIKDTDQFIASFYKNDLIKLNGDLYKIIGVNSDDRNIIELDYYD
IKYKDYCEINNIKGEPRIKKTIGKKTESIEKFTTDVLGNLYLHSTEKAPQ
LIFKRGL.

Exemplary sRGN3.3 amino acid sequence lacking N-terminus methionine:

(SEQ ID NO: 5813)
NQKFILGLDIGITSVGYGLIDYETKNIIDAGVRLFPEANVENNEGRRSKR
GSRRLKRRRIHRLERVKLLLTEYDLINKEQIPTSNNPYQIRVKGLSEILS
KDELAIALLHLAKRRGIHNVDVAADKEETASDSLSTKDQINKNAKFLESR
YVCELQKERLENEGHVRGVENRFLTKDIVREAKKIIDTQMQYYPEIDETF
KEKYISLVETRREYFEGPGQGSPFGWNGDLKKWYEMLMGHCTYFPQELRS
VKYAYSADLFNALNDLNNLIIQRDNSEKLEYHEKYHIIENVFKQKKKPTL
KQIAKEIGVNPEDIKGYRITKSGTPEFTSFKLFHDLKKVVKDHAILDDID
LLNQIAEILTIYQDKDSIVAELGQLEYLMSEADKQSISELTGYTGTHSLS
LKCMNMIIDELWHSSMNQMEVFTYLNMRPKKYELKGYQRIPTDMIDDAIL
SPVVKRTFIQSINVINKVIEKYGIPEDIIIELARENNSDDRKKFINNLQK
KNEATRKRINEIIGQTGNQNAKRIVEKIRLHDQQEGKCLYSLESIPLEDL
LNNPNHYEVDHIIPRSVSFDNSYHNKVLVKQSENSKKSNLTPYQYFNSGK
SKLSYNQFKQHILNLSKSQDRISKKKKEYLLEERDINKFEVQKEFINRNL
VDTRYATRELTSYLKAYFSANNMDVKVKTINGSFTNHLRKVWRFDKYRNH
GYKHHAEDALIIANADFLFKENKKLQNTNKILEKPTIENNTKKVTVEKEE
DYNNVFETPKLVEDIKQYRDYKFSHRVDKKPNRQLINDTLYSTRMKDEHD
YIVQTITDIYGKDNTNLKKQFNKNPEKFLMYQNDPKTFEKLSIIMKQYSD
EKNPLAKYYEETGEYLTKYSKKNNGPIVKKIKLLGNKVGNHLDVTNKYEN
STKKLVKLSIKNYRFDVYLTEKGYKFVTIAYLNVFKKDNYYYIPKDKYQE
LKEKKKIKDTDQFIASFYKNDLIKLNGDLYKIIGVNSDDRNIIELDYYDI
KYKDYCEINNIKGEPRIKKTIGKKTESIEKFTTDVLGNLYLHSTEKAPQL
IFKRGL.

Exemplary sRGN3.3 nickase amino acid sequence:

(SEQ ID NO: 5814)
MNQKFILGLDIGITSVGYGLIDYETKNIIDAGVRLFPEANVENNEGRRSK
RGSRRLKRRRIHRLERVKLLLTEYDLINKEQIPTSNNPYQIRVKGLSEIL
SKDELAIALLHLAKRRGIHNVDVAADKEETASDSLSTKDQINKNAKFLES
RYVCELQKERLENEGHVRGVENRFLTKDIVREAKKIIDTQMQYYPEIDET
FKEKYISLVETRREYFEGPGQGSPFGWNGDLKKWYEMLMGHCTYFPQELR
SVKYAYSADLFNALNDLNNLIIQRDNSEKLEYHEKYHIIENVFKQKKKPT
LKQIAKEIGVNPEDIKGYRITKSGTPEFTSFKLFHDLKKVVKDHAILDDI
DLLNQIAEILTIYQDKDSIVAELGQLEYLMSEADKQSISELTGYTGTHSL
SLKCMNMIIDELWHSSMNQMEVFTYLNMRPKKYELKGYQRIPTDMIDDAI
LSPVVKRTFIQSINVINKVIEKYGIPEDIIIELARENNSDDRKKFINNLQ
KKNEATRKRINEIIGQTGNQNAKRIVEKIRLHDQQEGKCLYSLESIPLED
LLNNPNHYEVDHIIPRSVSFDNSYHNKVLVKQSEASKKSNLTPYQYFNSG
KSKLSYNQFKQHILNLSKSQDRISKKKKEYLLEERDINKFEVQKEFINRN
LVDTRYATRELTSYLKAYFSANNMDVKVKTINGSFTNHLRKVWRFDKYRN
HGYKHHAEDALIIANADFLFKENKKLQNTNKILEKPTIENNTKKVTVEKE
EDYNNVFETPKLVEDIKQYRDYKFSHRVDKKPNRQLINDTLYSTRMKDEH
DYIVQTITDIYGKDNTNLKKQFNKNPEKFLMYQNDPKTFEKLSIIMKQYS
DEKNPLAKYYEETGEYLTKYSKKNNGPIVKKIKLLGNKVGNHLDVTNKYE
NSTKKLVKLSIKNYRFDVYLTEKGYKFVTIAYLNVFKKDNYYYIPKDKYQ
ELKEKKKIKDTDQFIASFYKNDLIKLNGDLYKIIGVNSDDRNIIELDYYD
IKYKDYCEINNIKGEPRIKKTIGKKTESIEKFTTDVLGNLYLHSTEKAQP
LIFKRGL.

Exemplary sRGN3.3 nickase amino acid sequence lacking N-terminus methionine:

(SEQ ID NO: 5815)
NQKFILGLDIGITSVGYGLIDYETKNIIDAGVRLFPEANVENNEGRRSKR
GSRRLKRRRIHRLERVKLLLTEYDLINKEQIPTSNNPYQIRVKGLSEILS
KDELAIALLHLAKRRGIHNVDVAADKEETASDSLSTKDQINKNAKFLESR
YVCELQKERLENEGHVRGVENRFLTKDIVREAKKIIDTQMQYYPEIDETF
KEKYISLVETRREYFEGPGQGSPFGWNGDLKKWYEMLMGHCTYFPQELRS
VKYAYSADLFNALNDLNNLIIQRDNSEKLEYHEKYHIIENVFKQKKKPTL
KQIAKEIGVNPEDIKGYRITKSGTPEFTSFKLFHDLKKVVKDHAILDDID
LLNQIAEILTIYQDKDSIVAELGQLEYLMSEADKQSISELTGYTGTHSLS
LKCMNMIIDELWHSSMNQMEVFTYLNMRPKKYELKGYQRIPTDMIDDAIL
SPVVKRTFIQSINVINKVIEKYGIPEDIIIELARENNSDDRKKFINNLQK
KNEATRKRINEIIGQTGNQNAKRIVEKIRLHDQQEGKCLYSLESIPLEDL
LNNPNHYEVDHIIPRSVSFDNSYHNKVLVKQSEASKKSNLTPYQYFNSGK
SKLSYNQFKQHILNLSKSQDRISKKKKEYLLEERDINKFEVQKEFINRNL
VDTRYATRELTSYLKAYFSANNMDVKVKTINGSFTNHLRKVWRFDKYRNH
GYKHHAEDALIIANADFLFKENKKLQNTNKILEKPTIENNTKKVTVEKEE
DYNNVFETPKLVEDIKQYRDYKFSHRVDKKPNRQLINDTLYSTRMKDEHD
YIVQTITDIYGKDNTNLKKQFNKNPEKFLMYQNDPKTFEKLSIIMKQYSD
EKNPLAKYYEETGEYLTKYSKKNNGPIVKKIKLLGNKVGNHLDVTNKYEN
STKKLVKLSIKNYRFDVYLTEKGYKFVTIAYLNVFKKDNYYYIPKDKYQE
LKEKKKIKDTDQFIASFYKNDLIKLNGDLYKIIGVNSDDRNIIELDYYDI
KYKDYCEINNIKGEPRIKKTIGKKTESIEKFTTDVLGNLYLHSTEKAPQL
IFKRGL.

In some embodiments, a Cas9 protein comprises a variant Cas9 protein containing one or more amino acid substitutions. In some embodiments, a wildtype Cas9 protein comprises a RuvC domain and an HNH domain. In some embodiments, a prime editor comprises a nuclease active Cas9 protein that may cleave both strands of a double stranded target DNA sequence. In some embodiments, the nuclease active Cas9 protein comprises a functional RuvC domain and a functional HNH domain. In some embodiments, a prime editor comprises a Cas9 nickase that can bind to a guide polynucleotide and recognize a target DNA, but can cleave only one strand of a double stranded target DNA. In some embodiments, the Cas9 nickase comprises only one functional RuvC domain or one functional HNH domain. In some embodiments, a prime editor comprises a Cas9 that has a non-functional HNH domain and a functional RuvC domain. In some embodiments, the prime editor can cleave the edit strand (i.e. the PAM strand), but not the non-edit strand of a double stranded target DNA sequence. In some embodiments, a prime editor comprises a Cas9 having a non-functional RuvC domain that can cleave the target strand (i.e. the non-PAM strand), but not the edit strand of a double stranded target DNA sequence. In some embodiments, a prime editor comprises a Cas9 that has neither a functional RuvC domain nor a functional HNH domain, which may not cleave any strand of a double stranded target DNA sequence.

In some embodiments, a prime editor comprises a Cas9 having a mutation in the RuvC domain that reduces or abolishes the nuclease activity of the RuvC domain. In some embodiments, the Cas9 comprise a mutation at amino acid D10 as compared to a wild type SpCas9 as set forth in SEQ ID NO:5783, or a corresponding mutation thereof. In some embodiments, the Cas9 comprise a D10A mutation as compared to a wild type SpCas9 as set forth in SEQ ID NO: 5783, or a corresponding mutation thereof. In some embodiments, the Cas9 polypeptide comprise a mutation at amino acid D10, G12, and/or G17 as compared to a wild type SpCas9 as set forth in SEQ ID NO: 5783, or a corresponding mutation thereof. In some embodiments, the Cas9 polypeptide comprise a D10A mutation, a G12A mutation, and/or a G17A mutation as compared to a wild type SpCas9 as set forth in SEQ ID NO: 5783, or a corresponding mutation thereof.

In some embodiments, a prime editor comprises a Cas9 polypeptide having a mutation in the HNH domain that reduces or abolishes the nuclease activity of the HNH domain. In some embodiments, the Cas9 polypeptide comprise a mutation at amino acid H840 as compared to a wild type SpCas9 as set forth in SEQ ID NO: 5783, or a corresponding mutation thereof. In some embodiments, the Cas9 polypeptide comprise a H840A mutation as compared to a wild type SpCas9 as set forth in SEQ ID NO: 5783, or a corresponding mutation thereof. In some embodiments, the Cas9 polypeptide comprise a mutation at amino acid E762, D839, H840, N854, N856, N863, H982, H983, A984, D986, and/or a A987 as compared to a wild type SpCas9 as set forth in SEQ ID NO: 5783, or a corresponding mutation thereof. In some embodiments, the Cas9 polypeptide comprise a E762A, D839A, H840A, N854A, N856A, N863A, H982A, H983A, A984A, and/or a D986A mutation as compared to a wild type SpCas9 as set forth in SEQ ID NO: 5783, or a corresponding mutation thereof.

In some embodiments, a prime editor comprises a Cas9 having one or more amino acid substitutions in both the HNH domain and the RuvC domain that reduce or abolish the nuclease activity of both the HNH domain and the RuvC domain. In some embodiments, the prime editor comprises a nuclease inactive Cas9, or a nuclease dead Cas9 (dCas9). In some embodiments, the dCas9 comprises a H840X substitution and a D10X mutation compared to a wild type SpCas9 as set forth in SEQ ID NO: 5783 or corresponding mutations thereof, wherein X is any amino acid other than H for the H840X substitution and any amino acid other than D for the D10X substitution. In some embodiments, the dead Cas9 comprises a H840A and a D10A mutation as compared to a wild type SpCas9 as set forth in SEQ ID NO: 5783, or corresponding mutations thereof.

In some embodiments, a prime editor comprises a Cas protein, e.g., Cas9, containing modifications that allow altered PAM recognition. In prime editing using a Cas-protein-based prime editor, a “protospacer adjacent motif (PAM)”, PAM sequence, or PAM-like motif, may be used to refer to a short DNA sequence immediately following the protospacer sequence on the PAM strand of the target gene. In some embodiments, the PAM is recognized by the Cas nuclease in the prime editor during prime editing. In certain embodiments, the PAM is required for target binding of the Cas protein. The specific PAM sequence required for Cas protein recognition may depend on the specific type of the Cas protein. A PAM can be 1, 2, 3, 4, 5, 6, 7, 8, 9, 10 or more nucleotides in length. In some embodiments, a PAM is between 2-6 nucleotides in length. In some embodiments, the PAM can be a 5′ PAM (i.e., located upstream of the 5′ end of the protospacer). In other embodiments, the PAM can be a 3′ PAM (i.e., located downstream of the 5′ end of the protospacer). In some embodiments, the Cas protein of a prime editor recognizes a canonical PAM, for example, a SpCas9 recognizes 5′-NGG-3′ PAM. In some embodiments, the Cas protein of a prime editor has altered or non-canonical PAM specificities. Exemplary PAM sequences and corresponding Cas variants are described in Table 1 below. It should be appreciated that for each of the variants provided, the Cas protein comprises one or more of the amino acid substitutions as indicated compared to a wild type Cas protein sequence, for example, the Cas9 as set forth in SEQ ID NO: 5783. The PAM motifs as shown in Table 1 below are in the order of 5′ to 3′. As used in PAM sequences in Table 1, “N” refers to any one of nucleotides A, G, C, and T, “R” refers to nucleotide A or G, and “Y” refers to nucleotide C or T.

TABLE 1
Cas protein variants and corresponding PAM
sequences

| Variant | PAM |
|---|---|
| spCas9 (wild type) | NGG, NGA, NAG |
| spCas9-VRVRFRR | NG |
| R1335V/L1111R/D1135V/ | |
| G1218R/E1219F/A1322R/ | |
| T1337R | |
| spCas9-VQR (D1135V/ | NGA |
| R1335Q/T1337R) | |
| spCas9-EQR (D1135E/ | NGA |
| R1335Q/T1337R) | |
| spCas9-VRER (D1135V/ | NGCG |
| G1218R/R1335E/T1337R) | |
| xCas9 (E480K, E543D, | NGN |
| E1219V, K294R, Q1256K, | |
| A262T, S409I, M694I) | |
| SluCa9 | NNGG |
| saCas9 | NNGRRT |
| | (SEQ ID |
| | NO: 5820), |
| | NNGRRN |
| | (SEQ ID |
| | NO: 5821) |
| saCas9-KKH (E782K, | NNNRRT |
| N968K, R1015H) | (SEQ ID |
| | NO: 5822) |
| spCas9-MQKSER (D1135M, | NGCG/NGCN |
| S1136Q, G1218K, E1219S, | |
| R1335E, T1337R) | |
| spCas9-LRKIQK (D1135L, | NGTN |
| S1136R, G1218K, E1219I, | |
| R1335Q, T1337K) | |
| spCas9-LRVSQK (D1135L, | NGTN |
| S1136R, G1218V, E1219S, | |
| R1335Q, T1337K) | |
| spCas9-LRVSQL(D1135L, | NGTN |
| S1136R, G1218V, E1219S, | |
| R1335Q, T1337L) | |
| Cpf1 | TTTV |
| Spy-Mac | NAA |
| NmCas9 PAM | NNNNGATT |
| | (SEQ ID |
| | NO: 5823) |
| StCas9 PAM | NNAGAAW |
| | (SEQ ID |
| | NO: 5824) |
| TdCas9 PAM | NAAAAC |
| | (SEQ ID |
| | NO: 5825) |

In some embodiments, a prime editor comprises a Cas9 polypeptide comprising one or mutations selected from the group consisting of: A61R, L111R, D1135V, R221K, A262T, R324L, N394K, S4091, S4091, E427G, E480K, M495V, N497A, Y515N, K526E, F539S, E543D), R654L, R661A, R661L, R691A, N692A, M694A, M6941, Q695A, H698A, R753G, M7631, K848A, K890N, Q926A, K1003A, R1060A, L111R, R1114G, D11135E, D11135L, D11135N, S1136W, V11139A, D11180G, G1218K, G1218R, G1218S, E1219Q, E1219V, E1219V, Q1221H, P1249S, E1253K, N1317R, A1320V, P1321S, A1322R, 11322V, D1332G, R1332N, A1332R, R1333K, R1333P, R1335L, R1335Q, R1335V, T1337N, T1337R, S1338T, H1349R, and any combinations thereof as compared to a wildtype SpCas9 polypeptide as set forth in SEQ ID NO: 5783.

In some embodiments, a prime editor comprises a SaCas9 polypeptide. In some embodiments, the SaCas9 polypeptide comprises one or more of mutations E782K, N968K, and R10115H as compared to a wild type SaCas9. In some embodiments, a prime editor comprises a FnCas9 polypeptide, for example, a wildtype FnCas9 polypeptide or a FnCas9 polypeptide comprising one or more of mutations E1369R, E1449H, or R1556A as compared to the wild type FnCas9. In some embodiments, a prime editor comprises a Sc Cas9, for example, a wild type ScCas9 or a ScCas9 polypeptide comprises one or more of mutations I367K, G368D, I369K, H371L, T375S, T376G, and T1227K as compared to the wild type ScCas9. In some embodiments, a prime editor comprises a St1 Cas9 polypeptide, a St3 Cas9 polypeptide, or a Slu Cas9 polypeptide.

In some embodiments, a prime editor comprises a Cas polypeptide that comprises a circular permutant Cas variant. For example, a Cas9 polypeptide of a prime editor may be engineered such that the N-terminus and the C-terminus of a Cas9 protein (e.g., a wild type Cas9 protein, or a Cas9 nickase) are topically rearranged to retain the ability to bind DNA when complexed with a guide RNA (gRNA). An exemplary circular permutant configuration may be N-terminus-[original C-terminus]-[original N-terminus]-C-terminus. Any of the Cas9 proteins described herein, including any variant, ortholog, or naturally occurring Cas9 or equivalent thereof, may be reconfigured as a circular permutant variant.

Prime editors described herein may also comprise Cas proteins other than Cas9. For example, a prime editor as described herein may comprise a Cas12a (Cpf1) polypeptide or functional variants thereof. In some embodiments, the Cas12a polypeptide comprises a mutation that reduces or abolishes the endonuclease domain of the Cas12a polypeptide. In some embodiments, the Cas12a polypeptide is a Cas12a nickase. In some embodiments, the Cas protein comprises an amino acid sequence that comprises at least about 50%, 60%, 70%, 80%, 90%, 91%, 92%, 93%, 94%, 95%, 96%, 97%, 98%, 99%, or 100% sequence identity to a naturally occurring Cas12a polypeptide.

In some embodiments, a prime editor comprises a Cas protein that is a Cas12b (C2c1) or a Cas12c (C2c3) polypeptide. In some embodiments, the Cas protein comprises an amino acid sequence that comprises at least about 50%, 60%, 70%, 80%, 90%, 91%, 92%, 93%, 94%, 95%, 96%, 97%, 98%, 99%, or 100% sequence identity to a naturally occurring Cas12b (C2c1) or Cas12c (C2c3) protein. In some embodiments, the Cas protein is a Cas12b nickase or a Cas12c nickase. In some embodiments, the Cas protein is a Cas12e, a Cas12d, a Cas13, Cas14a, Cas14b, Cas14c, Cas14d, Cas14e, Cas14f, Cas14g, Cas14h, Cas14u, or a CasΦ polypeptide. In some embodiments, the Cas protein comprises an amino acid sequence that comprises at least about 50%, 60%, 70%, 80%, 90%, 91%, 92%, 93%, 94%, 95%, 96%, 97%, 98%, 99%, or 100% sequence identity to a naturally-occurring Cas12e, Cas12d, Cas13, Cas14a, Cas14b, Cas14c, Cas14d, Cas14e, Cas14f, Cas14g, Cas14h, Cas14u, or CasΦ protein. In some embodiments, the Cas protein is a Cas12e, Cas12d, Cas13, or Cas Φ nickase.

In some embodiments, a prime editor further comprises additional polypeptide components, for example, a flap endonuclease (FEN, e.g., FEN1). In some embodiments, the flap endonuclease excises the 5′ single stranded DNA of the edit strand of the target gene and assists incorporation of the intended nucleotide edit into the target gene. In some embodiments, the FEN is linked or fused to another component. In some embodiments, the FEN is provided in trans, for example, as a separate polypeptide or polynucleotide encoding the FEN.

In some embodiments, a prime editor further comprises one or more nuclear localization sequence (NLS). In some embodiments, the NLS helps promote translocation of a protein into the cell nucleus. In some embodiments, a prime editor comprises a fusion protein, e.g., a fusion protein comprising a DNA binding domain and a DNA polymerase, that comprises one or more NLSs. In some embodiments, one or more polypeptides of the prime editor are fused to or linked to one or more NLSs. In some embodiments, the prime editor comprises a DNA binding domain and a DNA polymerase domain that are provided in trans, wherein the DNA binding domain and/or the DNA polymerase domain is fused or linked to one or more NLSs.

In certain embodiments, a prime editor or prime editing complex comprises at least one NLS. In some embodiments, a prime editor or prime editing complex comprises at least two NLSs. In embodiments with at least two NLSs, the NLSs can be the same NLS, or they can be different NLSs.

Any NLSs that are known in the art are also contemplated herein. The NLSs may be any naturally occurring NLS, or any non-naturally occurring NLS (e.g., an NLS with one or more mutations relative to a wild-type NLS). In some embodiments, the one or more NLSs of a prime editor comprise bipartite NLSs. In some embodiments, the one or more NLSs of a prime editor are rich in lysine and arginine residues. In some embodiments, the one or more NLSs of a prime editor comprise proline residues. Non-limiting examples of NLS sequences are provided in Table 2 below.

TABLE 2
Exemplary nuclear localization sequences

| | | SEQ |
|---|---|---|
| | | ID |
| Description | Sequence | NO: |
| NLS of SV40 | PKKKRKV | 5826 |
| Large T-AG | | |
| NLS | MKRTADGSEFESPKKKRKV | 5827 |
| NLS | MDSLLMNRRKFLYQFKNVRWAKGRRETYLC | 5828 |
| NLS of | AVKRPAATKKAGQAKKKKLD | 5829 |
| Nucleoplasmin | | |
| NLS of EGL-13 | MSRRRKANPTKLSENAKKLAKEVEN | 5830 |
| NLS of C-Myc | PAAKRVKLD | 5831 |
| NLS of Tus- | KLKIKRPVK | 5832 |
| protein | | |
| NLS of polyoma | VSRKRPRP | 5833 |
| large T-AG | | |
| NLS of | EGAPPAKRAR | 5834 |
| Hepatitis | | |
| D virus | | |
| antigen | | |
| NLS of | PPQPKKKPLDGE | 5835 |
| murine p53 | | |
| NLS of PE1 | SGGSKRTADGSEFEPKKKRKV | 5836 |
| and PE2 | | |

In some embodiments, a prime editing complex comprises a fusion protein comprising a DNA binding domain (e.g., Cas9 (H840A)) and a reverse transcriptase (e.g., a variant MMLV RT) having the following structure: [NLS]-[Cas9(H840A)]-[linker]-[MMLV_RT(D200N)(T330P)(L603W)(T306K)(W313F)], and a desired PEgRNA. In some embodiments, the prime editing complex comprises a prime editor fusion protein that has the amino acid sequence of SEQ ID NO: 5837 or 5838. The sequences and components of these exemplary prime editor fusion proteins which are shown as follows:

TABLE 34A
Exemplary prime editor fusion protein and
component sequences.

| SEQ | | |
|---|---|---|
| ID | DESCRIP- | |
| NO | TION | SEQUENCE |
| 5837 | Prime | MKRTADGSEFESPKKKRKVDKKYSIGLD |
| | Editor | IGTNSVGWAVITDEYKVPSKKFKVLGNT |
| | Structure: | DRHSIKKNLIGALLFDSGETAEATRLKR |
| | [N- | TARRRYTRRKNRICYLQEIFSNEMAKVD |
| | terminal | DSFFHRLEESFLVEEDKKHERHPIFGNI |
| | NLS]- | VDEVAYHEKYPTIYHLRKKLVDSTDKAD |
| | [Cas9 | LRLIYLALAHMIKFRGHFLIEGDLNPDN |
| | (H840A)]- | SDVDKLFIQLVQTYNQLFEENPINASGV |
| | [LINKER]- | DAKAILSARLSKSRRLENLIAQLPGEKK |
| | [MMLV_RT | NGLFGNLIALSLGLTPNFKSNFDLAEDA |
| | (D200N) | KLQLSKDTYDDDLDNLLAQIGDQYADLF |
| | (T330P) | LAAKNLSDAILLSDILRVNTEITKAPLS |
| | (L603W) | ASMIKRYDEHHQDLTLLKALVRQQLPEK |
| | (T306K) | YKEIFFDQSKNGYAGYIDGGASQEEFYK |
| | (W313F)]- | FIKPILEKMDGTEELLVKLNREDLLRKQ |
| | [C- | RTFDNGSIPHQIHLGELHAILRRQEDFY |
| | terminal | PFLKDNREKIEKILTFRIPYYVGPLARG |
| | linker | NSRFAWMTRKSEETITPWNFEEVVDKGA |
| | and NLS] | SAQSFIERMTNFDKNLPNEKVLPKHSLL |
| | | YEYFTVYNELTKVKYVTEGMRKPAFLSG |
| | | EQKKAIVDLLFKTNRKVTVKQLKEDYFK |
| | | KIECFDSVEISGVEDRFNASLGTYHDLL |
| | | KIIKDKDFLDNEENEDILEDIVLTLTLF |
| | | EDREMIEERLKTYAHLFDDKVMKQLKRR |
| | | RYTGWGRLSRKLINGIRDKQSGKTILDF |
| | | LKSDGFANRNFMQLIHDDSLTFKEDIQK |
| | | AQVSGQGDSLHEHIANLAGSPAIKKGIL |
| | | QTVKVVDELVKVMGRHKPENIVIEMARE |
| | | NQTTQKGQKNSRERMKRIEEGIKELGSQ |
| | | ILKEHPVENTQLQNEKLYLYYLQNGRDM |
| | | YVDQELDINRLSDYDVDAIVPQSFLKDD |
| | | SIDNKVLTRSDKNRGKSDNVPSEEVVKK |
| | | MKNYWRQLLNAKLITQRKFDNLTKAERG |
| | | GLSELDKAGFIKRQLVETRQITKHVAQI |
| | | LDSRMNTKYDENDKLIREVKVITLKSKL |
| | | VSDFRKDFQFYKVREINNYHHAHDAYLN |
| | | AVVGTALIKKYPKLESEFVYGDYKVYDV |
| | | RKMIAKSEQEIGKATAKYFFYSNIMNFF |
| | | KTEITLANGEIRKRPLIETNGETGEIVW |
| | | DKGRDFATVRKVLSMPQVNIVKKTEVQT |
| | | GGFSKESILPKRNSDKLIARKKDWDPKK |
| | | YGGFDSPTVAYSVLVVAKVEKGKSKKLK |
| | | SVKELLGITIMERSSFEKNPIDFLEAKG |
| | | YKEVKKDLIIKLPKYSLFELENGRKRML |
| | | ASAGELQKGNELALPSKYVNFLYLASHY |
| | | EKLKGSPEDNEQKQLFVEQHKHYLDEII |
| | | EQISEFSKRVILADANLDKVLSAYNKHR |
| | | DKPIREQAENIIHLFTLTNLGAPAAFKY |
| | | FDTTIDRKRYTSTKEVLDATLIHQSITG |
| | | LYETRIDLSQLGGD |
| | | [symbol] |
| | | [symbol] |
| | | TLNIEDEYRLHETSKEPDVSLGSTWLSD |
| | | FPQAWAETGGMGLAVRQAPLIIPLKATS |
| | | TPVSIKQYPMSQEARLGIKPHIQRLLDQ |
| | | GILVPCQSPWNTPLLPVKKPGTNDYRPV |
| | | QDLREVNKRVEDIHPTVPNPYNLLSGLP |
| | | PSHQWYTVLDLKDAFFCLRLHPTSQPLF |
| | | AFEWRDPEMGISGQLTWTRLPQGFKNSP |
| | | TLFNEALHRDLADFRIQHPDLILLQYVD |
| | | DLLLAATSELDCQQGTRALLQTLGNLGY |
| | | RASAKKAQICQKQVKYLGYLLKEGQRWL |
| | | TEARKETVMGQPTPKTPRQLREFLGKAG |
| | | FCRLFIPGFAEMAAPLYPLTKPGTLFNW |
| | | GPDQQKAYQEIKQALLTAPALGLPDLTK |
| | | PFELFVDEKQGYAKGVLTQKLGPWRRPV |
| | | AYLSKKLDPVAAGWPPCLRMVAAIAVLT |
| | | KDAGKLTMGQPLVILAPHAVEALVKQPP |
| | | DRWLSNARMTHYQALLLDTDRVQFGPVV |
| | | ALNPATLLPLPEEGLQHNCLDILAEAHG |
| | | TRPDLTDQPLPDADHTWYTDGSSLLQEG |
| | | QRKAGAAVTTETEVIWAKALPAGTSAQR |
| | | AELIALTQALKMAEGKKLNVYTDSRYAF |
| | | ATAHIHGEIYRRRGWLTSEGKEIKNKDE |
| | | ILALLKALFLPKRLSIIHCPGHQKGHSA |
| | | EARGNRMADQAARKAAITETPDTSTLLI |
| | | ENSSP |
| | | SGGSKRTADGSEFEPKKKRKV |
| 5827 | N | MKRTADGSEFESPKKKRKV |
| | terminal | |
| | NLS | |
| 5786 | SpCas9 | DKKYSIGLDIGTNSVGWAVITDEYKVPS |
| | (H840A) | KKFKVLGNTDRHSIKKNLIGALLFDSGE |
| | | TAEATRLKRTARRRYTRRKNRICYLQEI |
| | | FSNEMAKVDDSFFHRLEESFLVEEDKKH |
| | | ERHPIFGNIVDEVAYHEKYPTIYHLRKK |
| | | LVDSTDKADLRLIYLALAHMIKFRGHFL |
| | | IEGDLNPDNSDVDKLFIQLVQTYNQLFE |
| | | ENPINASGVDAKAILSARLSKSRRLENL |
| | | IAQLPGEKKNGLFGNLIALSLGLTPNFK |
| | | SNFDLAEDAKLQLSKDTYDDDLDNLLAQ |
| | | IGDQYADLFLAAKNLSDAILLSDILRVN |
| | | TEITKAPLSASMIKRYDEHHQDLTLLKA |
| | | LVRQQLPEKYKEIFFDQSKNGYAGYIDG |
| | | GASQEEFYKFIKPILEKMDGTEELLVKL |
| | | NREDLLRKQRTFDNGSIPHQIHLGELHA |
| | | ILRRQEDFYPFLKDNREKIEKILTFRIP |
| | | YYVGPLARGNSRFAWMTRKSEETITPWN |
| | | FEEVVDKGASAQSFIERMTNFDKNLPNE |
| | | KVLPKHSLLYEYFTVYNELTKVKYVTEG |
| | | MRKPAFLSGEQKKAIVDLLFKTNRKVTV |
| | | KQLKEDYFKKIECFDSVEISGVEDRFNA |
| | | SLGTYHDLLKIIKDKDFLDNEENEDILE |
| | | DIVLTLTLFEDREMIEERLKTYAHLFDD |
| | | KVMKQLKRRRYTGWGRLSRKLINGIRDK |
| | | QSGKTILDFLKSDGFANRNFMQLIHDDS |
| | | LTFKEDIQKAQVSGQGDSLHEHIANLAG |
| | | SPAIKKGILQTVKVVDELVKVMGRHKPE |
| | | NIVIEMARENQTTQKGQKNSRERMKRIE |
| | | EGIKELGSQILKEHPVENTQLQNEKLYL |
| | | YYLQNGRDMYVDQELDINRLSDYDVDAI |
| | | VPQSFLKDDSIDNKVLTRSDKNRGKSDN |
| | | VPSEEVVKKMKNYWRQLLNAKLITQRKF |
| | | DNLTKAERGGLSELDKAGFIKRQLVETR |
| | | QITKHVAQILDSRMNTKYDENDKLIREV |
| | | KVITLKSKLVSDFRKDFQFYKVREINNY |
| | | HHAHDAYLNAVVGTALIKKYPKLESEFV |
| | | YGDYKVYDVRKMIAKSEQEIGKATAKYF |
| | | FYSNIMNFFKTEITLANGEIRKRPLIET |
| | | NGETGEIVWDKGRDFATVRKVLSMPQVN |
| | | IVKKTEVQTGGFSKESILPKRNSDKLIA |
| | | RKKDWDPKKYGGFDSPTVAYSVLVVAKV |
| | | EKGKSKKLKSVKELLGITIMERSSFEKN |
| | | PIDFLEAKGYKEVKKDLIIKLPKYSLFE |
| | | LENGRKRMLASAGELQKGNELALPSKYV |
| | | NFLYLASHYEKLKGSPEDNEQKQLFVEQ |
| | | HKHYLDEIIEQISEFSKRVILADANLDK |
| | | VLSAYNKHRDKPIREQAENIIHLFTLTN |
| | | LGAPAAFKYFDTTIDRKRYTSTKEVLDA |
| | | TLIHQSITGLYETRIDLSQLGGD |
| 5852 | LINKER | SGGSSGGSSGSETPGTSESATPESSGGS |
| | | SGGSS |
| 5842 | MMLV_ | TLNIEDEYRLHETSKEPDVSLGSTWLSD |
| | RT | FPQAWAETGGMGLAVRQAPLIIPLKATS |
| | (D200N) | TPVSIKQYPMSQEARLGIKPHIQRLLDQ |
| | (T330P) | GILVPCQSPWNTPLLPVKKPGTNDYRPV |
| | (L603W) | QDLREVNKRVEDIHPTVPNPYNLLSGLP |
| | (T306K) | PSHQWYTVLDLKDAFFCLRLHPTSQPLF |
| | (W313F) | AFEWRDPEMGISGQLTWTRLPQGFKNSP |
| | | TLFNEALHRDLADFRIQHPDLILLQYVD |
| | | DLLLAATSELDCQQGTRALLQTLGNLGY |
| | | RASAKKAQICQKQVKYLGYLLKEGQRWL |
| | | TEARKETVMGQPTPKTPRQLREFLGKAG |
| | | FCRLFIPGFAEMAAPLYPLTKPGTLFNW |
| | | GPDQQKAYQEIKQALLTAPALGLPDLTK |
| | | PFELFVDEKQGYAKGVLTQKLGPWRRPV |
| | | AYLSKKLDPVAAGWPPCLRMVAAIAVLT |
| | | KDAGKLTMGQPLVILAPHAVEALVKQPP |
| | | DRWLSNARMTHYQALLLDTDRVQFGPVV |
| | | ALNPATLLPLPEEGLQHNCLDILAEAHG |
| | | TRPDLTDQPLPDADHTWYTDGSSLLQEG |
| | | QRKAGAAVTTETEVIWAKALPAGTSAQR |
| | | AELIALTQALKMAEGKKLNVYTDSRYAF |
| | | ATAHIHGEIYRRRGWLTSEGKEIKNKDE |
| | | ILALLKALFLPKRLSIIHCPGHQKGHSA |
| | | EARGNRMADQAARKAAITETPDTSTLLI |
| | | ENSSP |
| 5836 | C- | SGGSKRTADGSEFEPKKKRKV |
| | Terminal | |
| | linker | |
| | and NLS | |

TABLE 34B
Exemplary prime editor fusion protein and
component sequences.

| SEQ | | |
|---|---|---|
| ID | DESCRIP- | |
| NO | TION | SEQUENCE |
| 5838 | PEmax | MKRTADGSEFESPKKKRKV |
| | Prime | DKKYSIGLDIGTNSVGWAVITDEYKVP |
| | Editor | SKKFKVLGNTDRHSIKKNLIGALLFDS |
| | structure: | GETAEATRLKRTARRRYTRRKNRICYL |
| | [N- | QEIFSNEMAKVDDSFFHRLEESFLVEE |
| | terminal | DKKHERHPIFGNIVDEVAYHEKYPTIY |
| | NLS]- | HLRKKLVDSTDKADLRLIYLALAHMIK |
| | [Cas9 | FRGHFLIEGDLNPDNSDVDKLFIQLVQ |
| | ((R221K | TYNQLFEENPINASGVDAKAILSARLS |
| | N394K | KSRKLENLIAQLPGEKKNGLFGNLIAL |
| | H840A)]- | SLGLTPNFKSNFDLAEDAKLQLSKDTY |
| | [SGGSx2- | DDDLDNLLAQIGDQYADLFLAAKNLSD |
| | bpSV40NLS- | AILLSDILRVNTEITKAPLSASMIKRY |
| | SGGSx2]- | DEHHQDLTLLKALVRQQLPEKYKEIFF |
| | [MMLV_RT | DQSKNGYAGYIDGGASQEEFYKFIKPI |
| | (D200N) | LEKMDGTEELLVKLKREDLLRKQRTFD |
| | (T330P) | NGSIPHQIHLGELHAILRRQEDFYPFL |
| | (L603W) | KDNREKIEKILTFRIPYYVGPLARGNS |
| | (T306K) | RFAWMTRKSEETITPWNFEEVVDKGAS |
| | (W313F)]- | AQSFIERMTNFDKNLPNEKVLPKHSLL |
| | [C- | YEYFTVYNELTKVKYVTEGMRKPAFLS |
| | terminal | GEQKKAIVDLLFKTNRKVTVKQLKEDY |
| | linker | FKKIECFDSVEISGVEDRFNASLGTYH |
| | and NLS- | DLLKIIKDKDFLDNEENEDILEDIVLT |
| | linker- | LTLFEDREMIEERLKTYAHLFDDKVMK |
| | NLS2] | QLKRRRYTGWGRLSRKLINGIRDKQSG |
| | | KTILDFLKSDGFANRNFMQLIHDDSLT |
| | | FKEDIQKAQVSGQGDSLHEHIANLAGS |
| | | PAIKKGILQTVKVVDELVKVMGRHKPE |
| | | NIVIEMARENQTTQKGQKNSRERMKRI |
| | | EEGIKELGSQILKEHPVENTQLQNEKL |
| | | YLYYLQNGRDMYVDQELDINRLSDYDV |
| | | DAIVPQSFLKDDSIDNKVLTRSDKNRG |
| | | KSDNVPSEEVVKKMKNYWRQLLNAKLI |
| | | TQRKFDNLTKAERGGLSELDKAGFIKR |
| | | QLVETRQITKHVAQILDSRMNTKYDEN |
| | | DKLIREVKVITLKSKLVSDFRKDFQFY |
| | | KVREINNYHHAHDAYLNAVVGTALIKK |
| | | YPKLESEFVYGDYKVYDVRKMIAKSEQ |
| | | EIGKATAKYFFYSNIMNFFKTEITLAN |
| | | GEIRKRPLIETNGETGEIVWDKGRDFA |
| | | TVRKVLSMPQVNIVKKTEVQTGGFSKE |
| | | SILPKRNSDKLIARKKDWDPKKYGGFD |
| | | SPTVAYSVLVVAKVEKGKSKKLKSVKE |
| | | LLGITIMERSSFEKNPIDFLEAKGYKE |
| | | VKKDLIIKLPKYSLFELENGRKRMLAS |
| | | AGELQKGNELALPSKYVNFLYLASHYE |
| | | KLKGSPEDNEQKQLFVEQHKHYLDEII |
| | | EQISEFSKRVILADANLDKVLSAYNKH |
| | | RDKPIREQAENIIHLFTLTNLGAPAAF |
| | | KYFDTTIDRKRYTSTKEVLDATLIHQS |
| | | ITGLYETRIDLSQLGGD |
| | | [symbol] |
| | | [symbol] |
| | | TLNIEDEYRLHETSKEPDVSLGSTWLS |
| | | DFPQAWAETGGMGLAVRQAPLIIPLKA |
| | | TSTPVSIKQYPMSQEARLGIKPHIQRL |
| | | LDQGILVPCQSPWNTPLLPVKKPGTND |
| | | YRPVQDLREVNKRVEDIHPTVPNPYNL |
| | | LSGLPPSHQWYTVLDLKDAFFCLRLHP |
| | | TSQPLFAFEWRDPEMGISGQLTWTRLP |
| | | QGFKNSPTLFNEALHRDLADFRIQHPD |
| | | LILLQYVDDLLLAATSELDCQQGTRAL |
| | | LQTLGNLGYRASAKKAQICQKQVKYLG |
| | | YLLKEGQRWLTEARKETVMGQPTPKTP |
| | | RQLREFLGKAGFCRLFIPGFAEMAAPL |
| | | YPLTKPGTLFNWGPDQQKAYQEIKQAL |
| | | LTAPALGLPDLTKPFELFVDEKQGYAK |
| | | GVLTQKLGPWRRPVAYLSKKLDPVAAG |
| | | WPPCLRMVAAIAVLTKDAGKLTMGQPL |
| | | VILAPHAVEALVKQPPDRWLSNARMTH |
| | | YQALLLDTDRVQFGPVVALNPATLLPL |
| | | PEEGLQHNCLDILAEAHGTRPDLTDQP |
| | | LPDADHTWYTDGSSLLQEGQRKAGAAV |
| | | TTETEVIWAKALPAGTSAQRAELIALT |
| | | QALKMAEGKKLNVYTDSRYAFATAHIH |
| | | GEIYRRRGWLTSEGKEIKNKDEILALL |
| | | KALFLPKRLSIIHCPGHQKGHSAEARG |
| | | NRMADQAARKAAITETPDTSTLLIENS |
| | | SP |
| | | SGGSKRTADGSEFESPKKKRKVGS |
| | | GPAAKRVKLD |
| 5839 | PEmax- | MKRTADGSEFESPKKKRKV |
| | N- | |
| | terminal | |
| | bpSV40NLS | |
| 5840 | PEmax- | DKKYSIGLDIGTNSVGWAVITDEYKVP |
| | CAS9 | SKKFKVLGNTDRHSIKKNLIGALLFDS |
| | (R221K | GETAEATRLKRTARRRYTRRKNRICYL |
| | N394K | QEIFSNEMAKVDDSFFHRLEESFLVEE |
| | H840A) | DKKHERHPIFGNIVDEVAYHEKYPTIY |
| | (not | HLRKKLVDSTDKADLRLIYLALAHMIK |
| | including | FRGHFLIEGDLNPDNSDVDKLFIQLVQ |
| | N- | TYNQLFEENPINASGVDAKAILSARLS |
| | terminal | KSRKLENLIAQLPGEKKNGLFGNLIAL |
| | Met in | SLGLTPNFKSNFDLAEDAKLQLSKDTY |
| | Cas9) | DDDLDNLLAQIGDQYADLFLAAKNLSD |
| | | AILLSDILRVNTEITKAPLSASMIKRY |
| | | DEHHQDLTLLKALVRQQLPEKYKEIFF |
| | | DQSKNGYAGYIDGGASQEEFYKFIKPI |
| | | LEKMDGTEELLVKLKREDLLRKQRTFD |
| | | NGSIPHQIHLGELHAILRRQEDFYPFL |
| | | KDNREKIEKILTFRIPYYVGPLARGNS |
| | | RFAWMTRKSEETITPWNFEEVVDKGAS |
| | | AQSFIERMTNFDKNLPNEKVLPKHSLL |
| | | YEYFTVYNELTKVKYVTEGMRKPAFLS |
| | | GEQKKAIVDLLFKTNRKVTVKQLKEDY |
| | | FKKIECFDSVEISGVEDRFNASLGTYH |
| | | DLLKIIKDKDFLDNEENEDILEDIVLT |
| | | LTLFEDREMIEERLKTYAHLFDDKVMK |
| | | QLKRRRYTGWGRLSRKLINGIRDKQSG |
| | | KTILDFLKSDGFANRNFMQLIHDDSLT |
| | | FKEDIQKAQVSGQGDSLHEHIANLAGS |
| | | PAIKKGILQTVKVVDELVKVMGRHKPE |
| | | NIVIEMARENQTTQKGQKNSRERMKRI |
| | | EEGIKELGSQILKEHPVENTQLQNEKL |
| | | YLYYLQNGRDMYVDQELDINRLSDYDV |
| | | DAIVPQSFLKDDSIDNKVLTRSDKNRG |
| | | KSDNVPSEEVVKKMKNYWRQLLNAKLI |
| | | TQRKFDNLTKAERGGLSELDKAGFIKR |
| | | QLVETRQITKHVAQILDSRMNTKYDEN |
| | | DKLIREVKVITLKSKLVSDFRKDFQFY |
| | | KVREINNYHHAHDAYLNAVVGTALIKK |
| | | YPKLESEFVYGDYKVYDVRKMIAKSEQ |
| | | EIGKATAKYFFYSNIMNFFKTEITLAN |
| | | GEIRKRPLIETNGETGEIVWDKGRDFA |
| | | TVRKVLSMPQVNIVKKTEVQTGGFSKE |
| | | SILPKRNSDKLIARKKDWDPKKYGGFD |
| | | SPTVAYSVLVVAKVEKGKSKKLKSVKE |
| | | LLGITIMERSSFEKNPIDFLEAKGYKE |
| | | VKKDLIIKLPKYSLFELENGRKRMLAS |
| | | AGELQKGNELALPSKYVNFLYLASHYE |
| | | KLKGSPEDNEQKQLFVEQHKHYLDEII |
| | | EQISEFSKRVILADANLDKVLSAYNKH |
| | | RDKPIREQAENIIHLFTLTNLGAPAAF |
| | | KYFDTTIDRKRYTSTKEVLDATLIHQS |
| | | ITGLYETRIDLSQLGGD |
| 5841 | PEmax- | SGGSSGGSKRTADGSEFESPKKKRKVS |
| | SGGSx2- | GGSSGGS |
| | bpSV40NLS- | |
| | SGGSx2 | |
| | linker | |
| 5842 | PEmax- | TLNIEDEYRLHETSKEPDVSLGSTWLS |
| | MMLV RT | DFPQAWAETGGMGLAVRQAPLIIPLKA |
| | D200N | TSTPVSIKQYPMSQEARLGIKPHIQRL |
| | T330P | LDQGILVPCQSPWNTPLLPVKKPGTND |
| | L603W | YRPVQDLREVNKRVEDIHPTVPNPYNL |
| | T306K | LSGLPPSHQWYTVLDLKDAFFCLRLHP |
| | W313F | TSQPLFAFEWRDPEMGISGQLTWTRLP |
| | | QGFKNSPTLFNEALHRDLADFRIQHPD |
| | | LILLQYVDDLLLAATSELDCQQGTRAL |
| | | LQTLGNLGYRASAKKAQICQKQVKYLG |
| | | YLLKEGQRWLTEARKETVMGQPTPKTP |
| | | RQLREFLGKAGFCRLFIPGFAEMAAPL |
| | | YPLTKPGTLFNWGPDQQKAYQEIKQAL |
| | | LTAPALGLPDLTKPFELFVDEKQGYAK |
| | | GVLTQKLGPWRRPVAYLSKKLDPVAAG |
| | | WPPCLRMVAAIAVLTKDAGKLTMGQPL |
| | | VILAPHAVEALVKQPPDRWLSNARMTH |
| | | YQALLLDTDRVQFGPVVALNPATLLPL |
| | | PEEGLQHNCLDILAEAHGTRPDLTDQP |
| | | LPDADHTWYTDGSSLLQEGQRKAGAAV |
| | | TTETEVIWAKALPAGTSAQRAELIALT |
| | | QALKMAEGKKLNVYTDSRYAFATAHIH |
| | | GEIYRRRGWLTSEGKEIKNKDEILALL |
| | | KALFLPKRLSIIHCPGHQKGHSAEARG |
| | | NRMADQAARKAAITETPDTSTLLIENS |
| | | SP |
| 5843 | PEmax- | SGGSKRTADGSEFESPKKKRKV |
| | C- | |
| | terminal | |
| | linker- | |
| | NLS | |
| 5844 | PEmax- | GSGPAAKRVKLD |
| | C- | |
| | terminal | |
| | linker- | |
| | NLS2 | |

Polypeptides comprising components of a prime editor may be fused via peptide linkers, or may be provided in trans relevant to each other. For example, a reverse transcriptase may be expressed, delivered, or otherwise provided as an individual component rather than as a part of a fusion protein with the DNA binding domain. In such cases, components of the prime editor may be associated through non-peptide linkages or co-localization functions. In some embodiments, a prime editor further comprises additional components capable of interacting with, associating with, or capable of recruiting other components of the prime editor or the prime editing system. For example, a prime editor may comprise an RNA-protein recruitment polypeptide that can associate with an RNA-protein recruitment RNA aptamer. In some embodiments, an RNA-protein recruitment polypeptide can recruit, or be recruited by, a specific RNA sequence. Non limiting examples of RNA-protein recruitment polypeptide and RNA aptamer pairs include a MS2 coat protein and a MS2 RNA hairpin, a PCP polypeptide and a PP7 RNA hairpin, a Com polypeptide and a Com RNA hairpin, a Ku protein and a telomerase Ku binding RNA motif, and a Sm7 protein and a telomerase Sm7 binding RNA motif. In some embodiments, the prime editor comprises a DNA binding domain fused or linked to an RNA-protein recruitment polypeptide. In some embodiments, the prime editor comprises a DNA polymerase domain fused or linked to an RNA-protein recruitment polypeptide. In some embodiments, the DNA binding domain and the DNA polymerase domain fused to the RNA-protein recruitment polypeptide, or the DNA binding domain fused to the RNA-protein recruitment polypeptide and the DNA polymerase domain are co-localized by the corresponding RNA-protein recruitment RNA aptamer of the RNA-protein recruitment polypeptide. In some embodiments, the corresponding RNA-protein recruitment RNA aptamer fused or linked to a portion of the PEgRNA or ngRNA. For example, an MS2 coat protein fused or linked to the DNA polymerase and a MS2 hairpin installed on the PEgRNA for co-localization of the DNA polymerase and the RNA-guided DNA binding domain (e.g., a Cas9 nickase).

In certain embodiments, components of a prime editor are directly fused to each other. In certain embodiments, components of a prime editor are associated to each other via a linker.

As used herein, a linker can be any chemical group or a molecule linking two molecules or moieties, e.g., a DNA binding domain and a polymerase domain of a prime editor. In some embodiments, a linker is an organic molecule, group, polymer, or chemical moiety. In some embodiments, the linker comprises a non-peptide moiety. The linker may be as simple as a covalent bond, or it may be a polymeric linker many atoms in length, for example, a polynucleotide sequence. In certain embodiments, the linker is a covalent bond (e.g., a carbon-carbon bond, disulfide bond, carbon-heteroatom bond, etc.).

In certain embodiments, two or more components of a prime editor are linked to each other by a peptide linker. In some embodiments, a peptide linker is 5-100 amino acids in length, for example, 2, 3, 4, 5, 6, 7, 8, 9, 10, 11, 12, 13, 14, 15, 16, 17, 18, 19, 20, 21, 22, 23, 24, 25, 26, 27, 28, 29, 30, 30-35, 35-40, 40-45, 45-50, 50-60, 60-70, 70-80, 80-90, 90-100, 100-150, or 150-200 amino acids in length. In some embodiments, the peptide linker is 16 amino acids in length, 24 amino acids in length, 64 amino acids in length, or 96 amino acids in length.

In some embodiments, the linker comprises the amino acid sequence (GGGGS)n (SEQ ID NO: 5845), (G)n (SEQ ID NO: 5846), (EAAAK)n (SEQ ID NO: 5847), (GGS)n (SEQ ID NO: 5848), (SGGS)n (SEQ ID NO: 5849), (XP)n (SEQ ID NO: 5850), or any combination thereof, wherein n is independently an integer between 1 and 30, and wherein X is any amino acid. In some embodiments, the linker comprises the amino acid sequence (GGS)n (SEQ ID NO: 5903), wherein n is 1, 3, or 7. In some embodiments, the linker comprises the amino acid sequence SGSETPGTSESATPES (SEQ ID NO: 5851). In some embodiments, the linker comprises the amino acid sequence SGGSSGGSSGSETPGTSESATPESSGGSSGGS (SEQ ID NO: 5852). In some embodiments, the linker comprises the amino acid sequence SGGSGGSGGS (SEQ ID NO: 5854). In some embodiments, the linker comprises the amino acid sequence SGGS (SEQ ID NO: 5855). In other embodiments, the linker comprises the amino acid sequence SGGSSGGSSGSETPGTSESATPESAGSYPYDVPDYAGSAAPAAKKKKLDGSGSGGSSGGS (SEQ ID NO: 5856).

In certain embodiments, two or more components of a prime editor are linked to each other by a non-peptide linker. In some embodiments, the linker is a carbon-nitrogen bond of an amide linkage. In certain embodiments, the linker is a cyclic or acyclic, substituted or unsubstituted, branched or unbranched aliphatic or heteroaliphatic linker. In certain embodiments, the linker is polymeric (e.g., polyethylene, polyethylene glycol, polyamide, polyester, etc.). In certain embodiments, the linker comprises a monomer, dimer, or polymer of aminoalkanoic acid. In certain embodiments, the linker comprises an aminoalkanoic acid (e.g., glycine, ethanoic acid, alanine, beta-alanine, 3-aminopropanoic acid, 4-aminobutanoic acid, 5-pentanoic acid, etc.). In certain embodiments, the linker comprises a monomer, dimer, or polymer of aminohexanoic acid (Ahx). In certain embodiments, the linker is based on a carbocyclic moiety (e.g., cyclopentane, cyclohexane). In other embodiments, the linker comprises a polyethylene glycol moiety (PEG). In certain embodiments, the linker comprises an aryl or heteroaryl moiety. In certain embodiments, the linker is based on a phenyl ring. The linker may include functionalized moieties to facilitate attachment of a nucleophile (e.g., thiol, amino) from the peptide to the linker. Any electrophile may be used as part of the linker. Exemplary electrophiles include, but are not limited to, activated esters, activated amides, Michael acceptors, alkyl halides, aryl halides, acyl halides, and isothiocyanates.

Components of a prime editor may be connected to each other in any order. In some embodiments, the DNA binding domain and the DNA polymerase domain of a prime editor may be fused to form a fusion protein, or may be joined by a peptide or protein linker, in any order from the N terminus to the C terminus. In some embodiments, a prime editor comprises a DNA binding domain fused or linked to the C-terminal end of a DNA polymerase domain. In some embodiments, a prime editor comprises a DNA binding domain fused or linked to the N-terminal end of a DNA polymerase domain. In some embodiments, the prime editor comprises a fusion protein comprising the structure NH2-[DNA binding domain]-[polymerase]-COOH; or NH2-[polymerase]-[DNA binding domain]-COOH, wherein each instance of “]-[” indicates the presence of an optional linker sequence. In some embodiments, a prime editor comprises a fusion protein and a DNA polymerase domain provided in trans, wherein the fusion protein comprises the structure NH2-[DNA binding domain]-[RNA-protein recruitment polypeptide]-COOH. In some embodiments, a prime editor comprises a fusion protein and a DNA binding domain provided in trans, wherein the fusion protein comprises the structure NH2-[DNA polymerase domain]-[RNA-protein recruitment polypeptide]-COOH.

In addition, the NLSs may be expressed as part of a prime editor complex. The location of the NLS fusion can be at the N-terminus, the C-terminus, or positioned anywhere within a sequence of a prime editor or a component thereof (e.g., inserted between the DNA-binding domain and the DNA polymerase domain of a prime editor fusion protein, between the DNA binding domain and a linker sequence, between a DNA polymerase and a linker sequence, between two linker sequences of a prime editor fusion protein or a component thereof, in either N-terminus to C-terminus or C-terminus to N-terminus order). In some embodiments, a prime editor is fusion protein that comprises an NLS at the N terminus. In some embodiments, a prime editor is fusion protein that comprises an NLS at the C terminus. In some embodiments, a prime editor is fusion protein that comprises at least one NLS at both the N terminus and the C terminus. In some embodiments, the prime editor is a fusion protein that comprises two NLSs at the N terminus and/or the C terminus.

In some embodiments, a prime editor fusion protein, a polypeptide component of a prime editor, or a polynucleotide encoding the prime editor fusion protein or polypeptide component, may be split into an N-terminal half and a C-terminal half or polypeptides that encode the N-terminal half and the C terminal half, and provided to a target DNA in a cell separately. For example, in certain embodiments, a prime editor fusion protein may be split into a N-terminal and a C-terminal half for separate delivery in AAV vectors, and subsequently translated and colocalized in a target cell to reform the complete polypeptide or prime editor protein. In such cases, separate halves of a protein or a fusion protein may each comprise a split-intein to facilitate colocalization and reformation of the complete protein or fusion protein by the mechanism of intein facilitated trans splicing. In some embodiments, a prime editor comprises a N-terminal half fused to an intein-N, and a C-terminal half fused to an intein-C, or polynucleotides or vectors (e.g. AAV vectors) encoding each thereof. When delivered and/or expressed in a target cell, the intein-N and the intein-C can be excised via protein trans-splicing, resulting in a complete prime editor fusion protein in the target cell.

### PEgRNA for Editing of ATP7B Gene

The term “prime editing guide RNA”, or “PEgRNA”, refers to a guide polynucleotide that comprises one or more intended nucleotide edits for incorporation into the target DNA. In some embodiments, the PEgRNA associates with and directs a prime editor to incorporate the one or more intended nucleotide edits into the target gene via prime editing. “Nucleotide edit” or “intended nucleotide edit” refers to a specified deletion of one or more nucleotides at one specific position, insertion of one or more nucleotides at one specific position, substitution of a single nucleotide, or other alterations at one specific position to be incorporated into the sequence of the target gene. Intended nucleotide edit may refer to the edit on the editing template as compared to the sequence on the target strand of the target gene or may refer to the edit encoded by the editing template on the newly synthesized single stranded DNA that replaces the editing target sequence, as compared to the editing target sequence. In some embodiments, a PEgRNA comprises a spacer sequence that is complementary or substantially complementary to a search target sequence on a target strand of the target gene. In some embodiments, the PEgRNA comprises a gRNA core that associates with a DNA binding domain, e.g., a CRISPR-Cas protein domain, of a prime editor. In some embodiments, the PEgRNA further comprises an extended nucleotide sequence comprising one or more intended nucleotide edits compared to the endogenous sequence of the target gene, wherein the extended nucleotide sequence may be referred to as an extension arm.

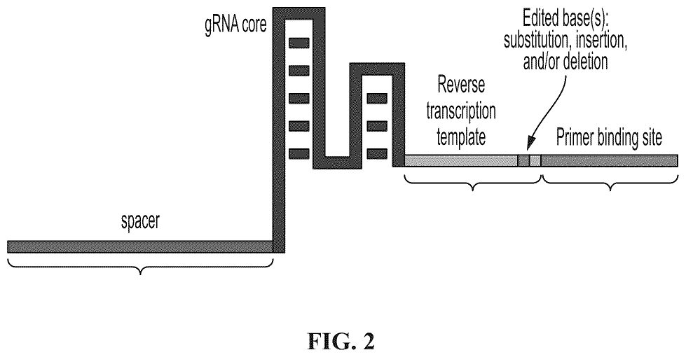

In certain embodiments, the extension arm comprises a primer binding site sequence (PBS) that can initiate target-primed DNA synthesis. In some embodiments, the PBS is complementary or substantially complementary to a free 3′ end on the edit strand of the target gene at a nick site generated by the prime editor. In some embodiments, the extension arm further comprises an editing template that comprises one or more intended nucleotide edits to be incorporated in the target gene by prime editing. In some embodiments, the editing template is a template for an RNA-dependent DNA polymerase domain or polypeptide of the prime editor, for example, a reverse transcriptase domain. The reverse transcriptase editing template may also be referred to herein as an RT template, or RTT. In some embodiments, the editing template comprises partial complementarity to an editing target sequence in the target gene. e.g., an ATP7B gene. In some embodiments, the editing template comprises substantial or partial complementarity to the editing target sequence except at the position of the intended nucleotide edits to be incorporated into the target gene. An exemplary architecture of a PEgRNA including its components is as demonstrated in FIG. 2.

In some embodiments, a PEgRNA includes only RNA nucleotides and forms an RNA polynucleotide. In some embodiments, a PEgRNA is a chimeric polynucleotide that includes both RNA and DNA nucleotides. For example, a PEgRNA can include DNA in the spacer sequence, the gRNA core, or the extension arm. In some embodiments, a PEgRNA comprises DNA in the spacer sequence. In some embodiments, the entire spacer sequence of a PEgRNA is a DNA sequence. In some embodiments, the PEgRNA comprises DNA in the gRNA core, for example, in a stem region of the gRNA core. In some embodiments, the PEgRNA comprises DNA in the extension arm, for example, in the editing template. An editing template that comprises a DNA sequence may serve as a DNA synthesis template for a DNA polymerase in a prime editor, for example, a DNA-dependent DNA polymerase. Accordingly, the PEgRNA may be a chimeric polynucleotide that comprises RNA in the spacer, gRNA core, and/or the PBS sequences and DNA in the editing template.

Components of a PEgRNA may be arranged in a modular fashion. In some embodiments, the spacer and the extension arm comprising a primer binding site sequence (PBS) and an editing template, e.g., a reverse transcriptase template (RTT), can be interchangeably located in the 5′ portion of the PEgRNA, the 3′ portion of the PEgRNA, or in the middle of the gRNA core. In some embodiments, a PEgRNA comprises a PBS and an editing template sequence in 5′ to 3′ order. In some embodiments, the gRNA core of a PEgRNA of this disclosure may be located in between a spacer and an extension arm of the PEgRNA. In some embodiments, the gRNA core of a PEgRNA may be located at the 3′ end of a spacer. In some embodiments, the gRNA core of a PEgRNA may be located at the 5′ end of a spacer. In some embodiments, the gRNA core of a PEgRNA may be located at the 3′ end of an extension arm. In some embodiments, the gRNA core of a PEgRNA may be located at the 5′ end of an extension arm. In some embodiments, the PEgRNA comprises, from 5′ to 3′: a spacer, a gRNA core, and an extension arm. In some embodiments, the PEgRNA comprises, from 5′ to 3′: a spacer, a gRNA core, an editing template, and a PBS. In some embodiments, the PEgRNA comprises, from 5′ to 3′: an extension arm, a spacer, and a gRNA core. In some embodiments, the PEgRNA comprises, from 5′ to 3′ an editing template, a PBS, a spacer, and a gRNA core.

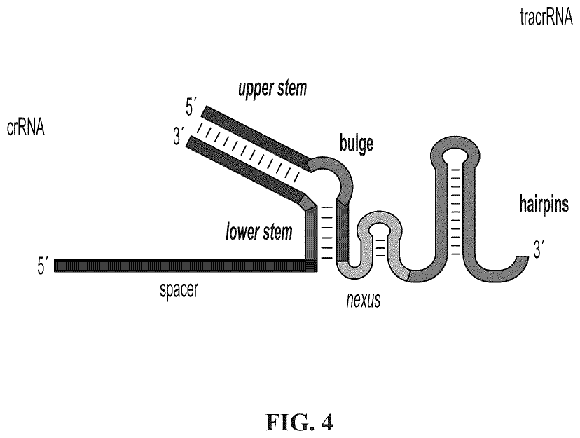

In some embodiments, a PEgRNA comprises a single polynucleotide molecule that comprises the spacer sequence, the gRNA core, and the extension arm. In some embodiments, a PEgRNA comprises multiple polynucleotide molecules, for example, two polynucleotide molecules. In some embodiments, a PEgRNA comprise a first polynucleotide molecule that comprises the spacer and a portion of the gRNA core, and a second polynucleotide molecule that comprises the rest of the gRNA core and the extension arm. In some embodiments, the gRNA core portion in the first polynucleotide molecule and the gRNA core portion in the second polynucleotide molecule are at least partly complementary to each other. In some embodiments, the PEgRNA may comprise a first polynucleotide comprising the spacer and a first portion of a gRNA core comprising, which may be also be referred to as a crRNA. In some embodiments, the PEgRNA comprise a second polynucleotide comprising a second portion of the gRNA core and the extension arm, wherein the second portion of the gRNA core may also be referred to as a trans-activating crRNA, or tracr RNA. In some embodiments, the crRNA portion and the tracr RNA portion of the gRNA core are at least partially complementary to each other. In some embodiments, the partially complementary portions of the crRNA and the tracr RNA form a lower stem, a bulge, and an upper stem, as exemplified in FIG. 4.

In some embodiments, a spacer sequence comprises a region that has substantial complementarity to a search target sequence on the target strand of a double stranded target DNA, e.g., an ATP7B gene. In some embodiments, the spacer sequence of a PEgRNA is identical or substantially identical to a protospacer sequence on the edit strand of the target gene (except that the protospacer sequence comprises thymine and the spacer sequence may comprise uracil). In some embodiments, the spacer sequence is at least about 70%, 75%, 80%, 85%, 90%, 95%, or 100% complementary to a search target sequence in the target gene. In some embodiments, the spacer comprises is substantially complementary to the search target sequence.

In some embodiments, the length of the spacer varies from about 10 to about 100 nucleotides. In some embodiments, the spacer is 16 nucleotides, 17 nucleotides, 18 nucleotides, 19 nucleotides, 20 nucleotides, 21 nucleotides, 22 nucleotides, 23 nucleotides, 24 nucleotides, or 25 nucleotides in length. In some embodiments, the spacer is from 15 nucleotides to 30 nucleotides in length, 15 to 25 nucleotides in length, 18 to 22 nucleotides in length, 10 to 20 nucleotides in length, or 20 to 30 nucleotides in length. In some embodiments, the spacer is 16 to 22 nucleotides in length. In some embodiments, the spacer is 16 to 20 nucleotides in length. In some embodiments, the spacer is 17 to 18 nucleotides in length. In some embodiments, the spacer is 20 nucleotides in length.

As used herein in a PEgRNA or a nick guide RNA sequence, or fragments thereof such as a spacer, PBS, or RTT sequence, unless indicated otherwise, it should be appreciated that the letter “T” or “thymine” indicates a nucleobase in a DNA sequence that encodes the PEgRNA or guide RNA sequence, and is intended to refer to a uracil (U) nucleobase of the PEgRNA or guide RNA or any chemically modified uracil nucleobase known in the art, such as 5-methoxyuracil.

The extension arm of a PEgRNA may comprise a primer binding site (PBS) and an editing template (e.g., an RTT). The extension arm may be partially complementary to the spacer. In some embodiments, the editing template (e.g., RTT) is partially complementary to the spacer. In some embodiments, the editing template (e.g., RTT) and the primer binding site (PBS) are each partially complementary to the spacer.

An extension arm of a PEgRNA may comprise a primer binding site sequence (PBS, or PBS sequence) that comprises complementarity to and can hybridize with a free 3′ end of a single stranded DNA in the target gene (e.g. the ATP7B gene) generated by nicking with a prime editor at the nick site on the PAM strand. The length of the PBS sequence may vary depending on, e.g., the prime editor components, the search target sequence and other components of the PEgRNA. In some embodiments, the PBS is about 3 to 19 nucleotides in length. in some embodiments, the PBS is about 3 to 17 nucleotides in length. In some embodiments, the PBS is about 4 to 16 nucleotides, about 6 to 16 nucleotides, about 6 to 18 nucleotides, about 6 to 20 nucleotides, about 8 to 20 nucleotides, about 10 to 20 nucleotides, about 12 to 20 nucleotides, about 14 to 20 nucleotides, about 16 to 20 nucleotides, or about 18 to 20 nucleotides in length. In some embodiments, the PBS is 8 to 17 nucleotides in length. In some embodiments, the PBS is 8 to 16 nucleotides in length. In some embodiments, the PBS is 8 to 15 nucleotides in length. In some embodiments, the PBS is 8 to 14 nucleotides in length. In some embodiments, the PBS is 8 to 13 nucleotides in length. In some embodiments, the PBS is 8 to 12 nucleotides in length. In some embodiments, the PBS is 8 to 11 nucleotides in length. In some embodiments, the PBS is 8 to 10 nucleotides in length. In some embodiments, the PBS is 8 or 9 nucleotides in length. In some embodiments, the PBS is 16 or 17 nucleotides in length. In some embodiments, the PBS is 15 to 17 nucleotides in length. In some embodiments, the PBS is 14 to 17 nucleotides in length. In some embodiments, the PBS is 13 to 17 nucleotides in length. In some embodiments, the PBS is 12 to 17 nucleotides in length. In some embodiments, the PBS is 11 to 17 nucleotides in length. In some embodiments, the PBS is 10 to 17 nucleotides in length. In some embodiments, the PBS is 9 to 17 nucleotides in length. In some embodiments, the PBS is about 7 to 15 nucleotides in length. In some embodiments, the PBS is 6, 7, 8, 9, 10, 11, 12, 13, 14, 15, 16, 17, 18, or 19 nucleotides in length. In some embodiments, the PBS is 8, 9, 10, 11, 12, 13, or 14 nucleotides in length.

The PBS may be complementary or substantially complementary to a DNA sequence in the edit strand of the target gene. By annealing with the edit strand at a free hydroxy group, e.g. a free 3′ end generated by prime editor nicking, the PBS may initiate synthesis of a new single stranded DNA encoded by the editing template at the nick site. In some embodiments, the PBS is at least about 70%, 75%, 80%, 85%, 90%, 95%, or 100% complementary to a region of the edit strand of the target gene (e.g., the ATP7B gene). In some embodiments, the PBS is perfectly complementary, or 100% complementary, to a region of the edit strand of the target gene (e.g., the ATP7B gene).

An extension arm of a PEgRNA may comprise an editing template that serves as a DNA synthesis template for the DNA polymerase in a prime editor during prime editing.

The length of an editing template may vary depending on, e.g., the prime editor components, the search target sequence and other components of the PEgRNA. In some embodiments, the editing template serves as a DNA synthesis template for a reverse transcriptase, and the editing template is referred to as a reverse transcription editing template (RTT).

The editing template (e.g., RTT), in some embodiments, is 2, 3, 4, 5, 6, 7, 8, 9, 10, 11, 12, 13, 14, 15, 16, 17, 18, 19, 20, 21, 22, 23, 24, or 25 nucleotides in length. In some embodiments, the RTT is 12, 13, 14, 15, 16, 17, 18, 19, 20, 21, 22, 23, 24, or 25 nucleotides in length. In some embodiments, the RTT is 16, 17, 18, 19, 20, 21, 22, 23, 24, 25, 26, 27, 28, 29, 30, 31, 32, 33, 34, 35, 36, 37, 38, 39, or 40 nucleotides in length. In some embodiments, the RTT is 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, or 80 nucleotides in length.

In some embodiments, the editing template (e.g., RTT) sequence is about 70%, 75%, 80%, 85%, 90%, 95%, or 99% complementary to the editing target sequence on the edit strand of the target gene. In some embodiments, the editing template sequence (e.g., RTT) is substantially complementary to the editing target sequence. In some embodiments, the editing template sequence (e.g., RTT) is complementary to the editing target sequence except at positions of the intended nucleotide edits to be incorporated in the target gene. In some embodiments, the editing template comprises a nucleotide sequence comprising about 85% to about 95% complementarity to an editing target sequence in the edit strand in the target gene (e.g., the ATP7B gene). In some embodiments, the editing template comprises about 86%, about 87%, about 88%, about 89%, about 90%, about 91%, about 92%, about 93%, about 94%, about 95%, about 96%, about 97%, about 98%, or about 99% complementarity to an editing target sequence in the edit strand of the target gene (e.g., the ATP7B gene).

An intended nucleotide edit in an editing template of a PEgRNA may comprise various types of alterations as compared to the target gene sequence. In some embodiments, the nucleotide edit is a single nucleotide substitution as compared to the target gene sequence. In some embodiments, the nucleotide edit is a deletion as compared to the target gene sequence. In some embodiments, the nucleotide edit is an insertion as compared to the target gene sequence. In some embodiments, the editing template comprises one to ten intended nucleotide edits as compared to the target gene sequence. In some embodiments, the editing template comprises one or more intended nucleotide edits as compared to the target gene sequence. In some embodiments, the editing template comprises two or more intended nucleotide edits as compared to the target gene sequence. In some embodiments, the editing template comprises three or more intended nucleotide edits as compared to the target gene sequence. In some embodiments, the editing template comprises four or more, five or more, or six or more intended nucleotide edits as compared to the target gene sequence. In some embodiments, the editing template comprises two single nucleotide substitutions, insertions, deletions, or any combination thereof, as compared to the target gene sequence. In some embodiments, the editing template comprises three single nucleotide substitutions, insertions, deletions, or any combination thereof, as compared to the target gene sequence. In some embodiments, the editing template comprises four, five, or six single nucleotide substitutions, insertions, deletions, or any combination thereof, as compared to the target gene sequence. In some embodiments, a nucleotide substitution comprises an adenine (A)-to-thymine (T) substitution. In some embodiments, a nucleotide substitution comprises an A-to-guanine (G) substitution. In some embodiments, a nucleotide substitution comprises an A-to-cytosine (C) substitution. In some embodiments, a nucleotide substitution comprises a T-A substitution. In some embodiments, a nucleotide substitution comprises a T-G substitution. In some embodiments, a nucleotide substitution comprises a T-C substitution. In some embodiments, a nucleotide substitution comprises a G-to-A substitution. In some embodiments, a nucleotide substitution comprises a G-to-T substitution. In some embodiments, a nucleotide substitution comprises a G-to-C substitution. In some embodiments, a nucleotide substitution comprises a C-to-A substitution. In some embodiments, a nucleotide substitution comprises a C-to-T substitution. In some embodiments, a nucleotide substitution comprises a C-to-G substitution.

In some embodiments, a nucleotide insertion is at least 5 nucleotides, at least 6 nucleotides, at least 7 nucleotides, at least 8 nucleotides, at least 9 nucleotides, at least 10 nucleotides, at least 11 nucleotides, at least 12 nucleotides, at least 13 nucleotides, at least 14 nucleotides, at least 15 nucleotides, at least 16 nucleotides, at least 17 nucleotides, at least 18 nucleotides, at least 19 nucleotides, or at least 20 nucleotides in length. In some embodiments, a nucleotide insertion is from 1 to 2 nucleotides, from 1 to 3 nucleotides, from 1 to 4 nucleotides, from 1 to 5 nucleotides, form 2 to 5 nucleotides, from 3 to 5 nucleotides, from 3 to 6 nucleotides, from 3 to 8 nucleotides, from 4 to 9 nucleotides, from 5 to 10 nucleotides, from 6 to 11 nucleotides, from 7 to 12 nucleotides, from 8 to 13 nucleotides, from 9 to 14 nucleotides, from 10 to 15 nucleotides, from 11 to 16 nucleotides, from 12 to 17 nucleotides, from 13 to 18 nucleotides, from 14 to 19 nucleotides, from 15 to 20 nucleotides in length. In some embodiments, a nucleotide insertion is a single nucleotide insertion. In some embodiments, a nucleotide insertion comprises insertion of two nucleotides.

The editing template of a PEgRNA may comprise one or more intended nucleotide edits, compared to the ATP7B gene to be edited. Position of the intended nucleotide edit(s) relevant to other components of the PEgRNA, or to particular nucleotides (e.g., mutations) in the ATP7B target gene may vary. In some embodiments, the nucleotide edit is in a region of the PEgRNA corresponding to or homologous to the protospacer sequence. In some embodiments, the nucleotide edit is in a region of the PEgRNA corresponding to a region of the ATP7B gene outside of the protospacer sequence.

In some embodiments, the position of a nucleotide edit incorporation in the target gene may be determined based on position of the protospacer adjacent motif (PAM). For instance, the intended nucleotide edit may be installed in a sequence corresponding to the protospacer adjacent motif (PAM) sequence. In some embodiments, a nucleotide edit in the editing template is at a position corresponding to the 5′ most nucleotide of the PAM sequence. In some embodiments, a nucleotide edit in the editing template is at a position corresponding to the 3′ most nucleotide of the PAM sequence. In some embodiments, position of an intended nucleotide edit in the editing template may be referred to by aligning the editing template with the partially complementary edit strand of the target gene, and referring to nucleotide positions on the editing strand where the intended nucleotide edit is incorporated. In some embodiments, a nucleotide edit is incorporated at a position corresponding to about 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 nucleotides upstream of the 5′ most nucleotide of the PAM sequence in the edit strand of the target gene. By 0 base pair upstream or downstream of a reference position, it is meant that the intended nucleotide is immediately upstream or downstream of the reference position. In some embodiments, a nucleotide edit is incorporated at a position corresponding to about 0 to 2 nucleotides, 0 to 4 nucleotides, 0 to 6 nucleotides, 0 to 8 nucleotides, 0 to 10 nucleotides, 2 to 4 nucleotides, 2 to 6 nucleotides, 2 to 8 nucleotides, 2 to 10 nucleotides, 2 to 12 nucleotides, 4 to 6 nucleotides, 4 to 8 nucleotides, 4 to 10 nucleotides, 4 to 12 nucleotides, 4 to 14 nucleotides, 6 to 8 nucleotides, 6 to 10 nucleotides, 6 to 12 nucleotides, 6 to 14 nucleotides, 6 to 16 nucleotides, 8 to 10 nucleotides, 8 to 12 nucleotides, 8 to 14 nucleotides, 8 to 16 nucleotides, 8 to 18 nucleotides, 10 to 12 nucleotides, 10 to 14 nucleotides, 10 to 16 nucleotides, 10 to 18 nucleotides, 10 to 20 nucleotides, 12 to 14 nucleotides, 12 to 16 nucleotides, 12 to 18 nucleotides, 12 to 20 nucleotides, 12 to 22 nucleotides, 14 to 16 nucleotides, 14 to 18 nucleotides, 14 to 20 nucleotides, 14 to 22 nucleotides, 14 to 24 nucleotides, 16 to 18 nucleotides, 16 to 20 nucleotides, 16 to 22 nucleotides, 16 to 24 nucleotides, 16 to 26 nucleotides, 18 to 20 nucleotides, 18 to 22 nucleotides, 18 to 24 nucleotides, 18 to 26 nucleotides, 18 to 28 nucleotides, 20 to 22 nucleotides, 20 to 24 nucleotides, 20 to 26 nucleotides, 20 to 28 nucleotides, or 20 to 30 nucleotides upstream of the 5′ most nucleotide of the PAM sequence. In some embodiments, the nucleotide edit is incorporated at a position corresponding to 3 nucleotides upstream of the 5′ most nucleotide of the PAM sequence. In some embodiments, the nucleotide edit in is incorporated at a position corresponding to 4 nucleotides upstream of the 5′ most nucleotide of the PAM sequence. In some embodiments, the nucleotide edit is incorporated at a position corresponding to 5 nucleotides upstream of the 5′ most nucleotide of the PAM sequence. In some embodiments, the nucleotide edit in the editing template is at a position corresponding to 6 nucleotides upstream of the 5′ most nucleotide of the PAM sequence.

In some embodiments, an intended nucleotide edit is incorporated at a position corresponding to about 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 nucleotides downstream of the 5′ most nucleotide of the PAM sequence in the edit strand of the target gene. In some embodiments, a nucleotide edit is incorporated at a position corresponding to about 0 to 2 nucleotides, 0 to 4 nucleotides, 0 to 6 nucleotides, 0 to 8 nucleotides, 0 to 10 nucleotides, 2 to 4 nucleotides, 2 to 6 nucleotides, 2 to 8 nucleotides, 2 to 10 nucleotides, 2 to 12 nucleotides, 4 to 6 nucleotides, 4 to 8 nucleotides, 4 to 10 nucleotides, 4 to 12 nucleotides, 4 to 14 nucleotides, 6 to 8 nucleotides, 6 to 10 nucleotides, 6 to 12 nucleotides, 6 to 14 nucleotides, 6 to 16 nucleotides, 8 to 10 nucleotides, 8 to 12 nucleotides, 8 to 14 nucleotides, 8 to 16 nucleotides, 8 to 18 nucleotides, 10 to 12 nucleotides, 10 to 14 nucleotides, 10 to 16 nucleotides, 10 to 18 nucleotides, 10 to 20 nucleotides, 12 to 14 nucleotides, 12 to 16 nucleotides, 12 to 18 nucleotides, 12 to 20 nucleotides, 12 to 22 nucleotides, 14 to 16 nucleotides, 14 to 18 nucleotides, 14 to 20 nucleotides, 14 to 22 nucleotides, 14 to 24 nucleotides, 16 to 18 nucleotides, 16 to 20 nucleotides, 16 to 22 nucleotides, 16 to 24 nucleotides, 16 to 26 nucleotides, 18 to 20 nucleotides, 18 to 22 nucleotides, 18 to 24 nucleotides, 18 to 26 nucleotides, 18 to 28 nucleotides, 20 to 22 nucleotides, 20 to 24 nucleotides, 20 to 26 nucleotides, 20 to 28 nucleotides, or 20 to 30 nucleotides downstream of the 5′ most nucleotide of the PAM sequence. In some embodiments, a nucleotide edit is incorporated at a position corresponding to 3 nucleotides downstream of the 5′ most nucleotide of the PAM sequence. In some embodiments, a nucleotide edit is incorporated at a position corresponding to 4 nucleotides downstream of the 5′ most nucleotide of the PAM sequence. In some embodiments, a nucleotide edit is incorporated at a position corresponding to 5 nucleotides downstream of the 5′ most nucleotide of the PAM sequence. In some embodiments, a nucleotide edit is incorporated at a position corresponding to 6 nucleotides downstream of the 5′ most nucleotide of the PAM sequence. By “upstream” and “downstream” it is intended to define relevant positions at least two regions or sequences in a nucleic acid molecule orientated in a 5′-to-3′ direction. For example, a first sequence is upstream of a second sequence in a DNA molecule where the first sequence is positioned 5′ to the second sequence. Accordingly, the second sequence is downstream of the first sequence.

When referred to within the PEgRNA, positions of the one or more intended nucleotide edits may be referred to relevant to components of the PEgRNA. For example, an intended nucleotide edit may be 5′ or 3′ to the PBS. In some embodiments, a PEgRNA comprises the structure, from 5′ to 3′: a spacer, a gRNA core, an editing template, and a PBS. In some embodiments, the intended nucleotide edit is 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 nucleotides upstream to the 5′ most nucleotide of the PBS. In some embodiments, the intended nucleotide edit is 0 to 2 nucleotides, 0 to 4 nucleotides, 0 to 6 nucleotides, 0 to 8 nucleotides, 0 to 10 nucleotides, 2 to 4 nucleotides, 2 to 6 nucleotides, 2 to 8 nucleotides, 2 to 10 nucleotides, 2 to 12 nucleotides, 4 to 6 nucleotides, 4 to 8 nucleotides, 4 to 10 nucleotides, 4 to 12 nucleotides, 4 to 14 nucleotides, 6 to 8 nucleotides, 6 to 10 nucleotides, 6 to 12 nucleotides, 6 to 14 nucleotides, 6 to 16 nucleotides, 8 to 10 nucleotides, 8 to 12 nucleotides, 8 to 14 nucleotides, 8 to 16 nucleotides, 8 to 18 nucleotides, 10 to 12 nucleotides, 10 to 14 nucleotides, 10 to 16 nucleotides, 10 to 18 nucleotides, 10 to 20 nucleotides, 12 to 14 nucleotides, 12 to 16 nucleotides, 12 to 18 nucleotides, 12 to 20 nucleotides, 12 to 22 nucleotides, 14 to 16 nucleotides, 14 to 18 nucleotides, 14 to 20 nucleotides, 14 to 22 nucleotides, 14 to 24 nucleotides, 16 to 18 nucleotides, 16 to 20 nucleotides, 16 to 22 nucleotides, 16 to 24 nucleotides, 16 to 26 nucleotides, 18 to 20 nucleotides, 18 to 22 nucleotides, 18 to 24 nucleotides, 18 to 26 nucleotides, 18 to 28 nucleotides, 20 to 22 nucleotides, 20 to 24 nucleotides, 20 to 26 nucleotides, 20 to 28 nucleotides, or 20 to 30 nucleotides upstream to the 5′ most nucleotide of the PBS.

The corresponding positions of the intended nucleotide edit incorporated in the target gene may also be referred to based on the nicking position (i.e. the nick site) generated by a prime editor based on sequence homology and complementarity. For example, in embodiments, the distance between the intended nucleotide edit to be incorporated into the target ATP7B gene and the nick site (also referred to as the “nick to edit distance”) may be determined by the position of the nick site and the position of the nucleotide(s) corresponding to the intended nucleotide edit(s), for example, by identifying sequence complementarity between the spacer and the search target sequence and sequence complementarity between the editing template and the editing target sequence. In certain embodiments, the position of the nucleotide edit can be in any position downstream of the nick site on the edit strand (or the PAM strand) generated by the prime editor, such that the distance between the nick site and the intended nucleotide edit is 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 nucleotides in length. In some embodiments, the position of the nucleotide edit is 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 nucleotides upstream of the nick site on the edit strand. In some embodiments, the position of the nucleotide edit is 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 nucleotides downstream of the nick site on the edit strand. In some embodiments, the position of the nucleotide edit is 0 base pair from the nick site on the edit strand, that is, the editing position is at the same position as the nick site. As used herein, the distance between the nick site and the nucleotide edit, for example, where the nucleotide edit comprises an insertion or deletion, refers to the 5′ most position of the nucleotide edit for a nick that creates a 3′ free end on the edit strand (i.e., the “near position” of the nucleotide edit to the nick site). Similarly, as used herein, the distance between the nick site and a PAM position edit, for example, where the nucleotide edit comprises an insertion, deletion, or substitution of two or more contiguous nucleotides, refers to the 5′ most position of the nucleotide edit and the 5′ most position of the PAM sequence.

In some embodiments, the editing template extends beyond a nucleotide edit to be incorporated to the target ATP7B gene sequence. For example, in some embodiments, the editing template comprises at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, or 80 nucleotides 3′ to the nucleotide edit to be incorporated to the target ATP7B gene sequence. In some embodiments, the editing template comprises 1 to 80 nucleotides 3′ to the nucleotide edit to be incorporated to the target ATP7B gene sequence.

In some embodiments, the editing template comprises 1 to 2 nucleotides 3′ to the nucleotide edit to be incorporated to the target ATP7B gene sequence. In some embodiments, the editing template comprises 1 to 3 nucleotides 3′ to the nucleotide edit to be incorporated to the target ATP7B gene sequence. In some embodiments, the editing template comprises 1 to 4 nucleotides 3′ to the nucleotide edit to be incorporated to the target ATP7B gene sequence. In some embodiments, the editing template comprises 1 to 5 nucleotides 3′ to the nucleotide edit to be incorporated to the target ATP7B gene sequence. In some embodiments, the editing template comprises 1 to 6 nucleotides 3′ to the nucleotide edit to be incorporated to the target ATP7B gene sequence. In some embodiments, the editing template comprises 1 to 7 nucleotides 3′ to the nucleotide edit to be incorporated to the target ATP7B gene sequence. In some embodiments, the editing template comprises 1 to 8 nucleotides 3′ to the nucleotide edit to be incorporated to the target ATP7B gene sequence. In some embodiments, the editing template comprises 1 to 9 nucleotides 3′ to the nucleotide edit to be incorporated to the target ATP7B gene sequence. In some embodiments, the editing template comprises 1 to 10 nucleotides 3′ to the nucleotide edit to be incorporated to the target ATP7B gene sequence. In some embodiments, the editing template comprises 1 to 11 nucleotides 3′ to the nucleotide edit to be incorporated to the target ATP7B gene sequence. In some embodiments, the editing template comprises 1 to 12 nucleotides 3′ to the nucleotide edit to be incorporated to the target ATP7B gene sequence. In some embodiments, the editing template comprises 1 to 13 nucleotides 3′ to the nucleotide edit to be incorporated to the target ATP7B gene sequence. In some embodiments, the editing template comprises 1 to 14 nucleotides 3′ to the nucleotide edit to be incorporated to the target ATP7B gene sequence. In some embodiments, the editing template comprises 1 to 15 nucleotides 3′ to the nucleotide edit to be incorporated to the target ATP7B gene sequence. In some embodiments, the editing template comprises 1 to 16 nucleotides 3′ to the nucleotide edit to be incorporated to the target ATP7B gene sequence. In some embodiments, the editing template comprises 1 to 17 nucleotides 3′ to the nucleotide edit to be incorporated to the target ATP7B gene sequence. In some embodiments, the editing template comprises 1 to 18 nucleotides 3′ to the nucleotide edit to be incorporated to the target ATP7B gene sequence. In some embodiments, the editing template comprises 1 to 19 nucleotides 3′ to the nucleotide edit to be incorporated to the target ATP7B gene sequence. In some embodiments, the editing template comprises 1 to 20 nucleotides 3′ to the nucleotide edit to be incorporated to the target ATP7B gene sequence. In some embodiments, the editing template comprises 1 to 21 nucleotides 3′ to the nucleotide edit to be incorporated to the target ATP7B gene sequence. In some embodiments, the editing template comprises 1 to 22 nucleotides 3′ to the nucleotide edit to be incorporated to the target ATP7B gene sequence. In some embodiments, the editing template comprises 1 to 23 nucleotides 3′ to the nucleotide edit to be incorporated to the target ATP7B gene sequence. In some embodiments, the editing template comprises 1 to 24 nucleotides 3′ to the nucleotide edit to be incorporated to the target ATP7B gene sequence. In some embodiments, the editing template comprises 1 to 25 nucleotides 3′ to the nucleotide edit to be incorporated to the target ATP7B gene sequence. In some embodiments, the editing template comprises 1 to 26 nucleotides 3′ to the nucleotide edit to be incorporated to the target ATP7B gene sequence. In some embodiments, the editing template comprises 1 to 27 nucleotides 3′ to the nucleotide edit to be incorporated to the target ATP7B gene sequence. In some embodiments, the editing template comprises 1 to 28 nucleotides 3′ to the nucleotide edit to be incorporated to the target ATP7B gene sequence. In some embodiments, the editing template comprises 1 to 29 nucleotides 3′ to the nucleotide edit to be incorporated to the target ATP7B gene sequence. In some embodiments, the editing template comprises 1 to 30 nucleotides 3′ to the nucleotide edit to be incorporated to the target ATP7B gene sequence. In some embodiments, the editing template comprises 1 to 31 nucleotides 3′ to the nucleotide edit to be incorporated to the target ATP7B gene sequence. In some embodiments, the editing template comprises 1 to 32 nucleotides 3′ to the nucleotide edit to be incorporated to the target ATP7B gene sequence. In some embodiments, the editing template comprises 1 to 33 nucleotides 3′ to the nucleotide edit to be incorporated to the target ATP7B gene sequence. In some embodiments, the editing template comprises 1 to 34 nucleotides 3′ to the nucleotide edit to be incorporated to the target ATP7B gene sequence. In some embodiments, the editing template comprises 1 to 35 nucleotides 3′ to the nucleotide edit to be incorporated to the target ATP7B gene sequence. In some embodiments, the editing template comprises 1 to 36 nucleotides 3′ to the nucleotide edit to be incorporated to the target ATP7B gene sequence. In some embodiments, the editing template comprises 1 to 37 nucleotides 3′ to the nucleotide edit to be incorporated to the target ATP7B gene sequence. In some embodiments, the editing template comprises 1 to 38 nucleotides 3′ to the nucleotide edit to be incorporated to the target ATP7B gene sequence. In some embodiments, the editing template comprises 1 to 39 nucleotides 3′ to the nucleotide edit to be incorporated to the target ATP7B gene sequence. In some embodiments, the editing template comprises 1 to 40 nucleotides 3′ to the nucleotide edit to be incorporated to the target ATP7B gene sequence. In some embodiments, the editing template comprises 1 to 41 nucleotides 3′ to the nucleotide edit to be incorporated to the target ATP7B gene sequence. In some embodiments, the editing template comprises 1 to 42 nucleotides 3′ to the nucleotide edit to be incorporated to the target ATP7B gene sequence. In some embodiments, the editing template comprises 1 to 43 nucleotides 3′ to the nucleotide edit to be incorporated to the target ATP7B gene sequence. In some embodiments, the editing template comprises 1 to 44 nucleotides 3′ to the nucleotide edit to be incorporated to the target ATP7B gene sequence. In some embodiments, the editing template comprises 1 to 45 nucleotides 3′ to the nucleotide edit to be incorporated to the target ATP7B gene sequence. In some embodiments, the editing template comprises 1 to 46 nucleotides 3′ to the nucleotide edit to be incorporated to the target ATP7B gene sequence. In some embodiments, the editing template comprises 1 to 47 nucleotides 3′ to the nucleotide edit to be incorporated to the target ATP7B gene sequence. In some embodiments, the editing template comprises 1 to 48 nucleotides 3′ to the nucleotide edit to be incorporated to the target ATP7B gene sequence. In some embodiments, the editing template comprises 1 to 49 nucleotides 3′ to the nucleotide edit to be incorporated to the target ATP7B gene sequence. In some embodiments, the editing template comprises 1 to 50 nucleotides 3′ to the nucleotide edit to be incorporated to the target ATP7B gene sequence. In some embodiments, the editing template comprises 1 to 51 nucleotides 3′ to the nucleotide edit to be incorporated to the target ATP7B gene sequence. In some embodiments, the editing template comprises 1 to 52 nucleotides 3′ to the nucleotide edit to be incorporated to the target ATP7B gene sequence. In some embodiments, the editing template comprises 1 to 53 nucleotides 3′ to the nucleotide edit to be incorporated to the target ATP7B gene sequence. In some embodiments, the editing template comprises 1 to 54 nucleotides 3′ to the nucleotide edit to be incorporated to the target ATP7B gene sequence. In some embodiments, the editing template comprises 1 to 55 nucleotides 3′ to the nucleotide edit to be incorporated to the target ATP7B gene sequence. In some embodiments, the editing template comprises 1 to 56 nucleotides 3′ to the nucleotide edit to be incorporated to the target ATP7B gene sequence. In some embodiments, the editing template comprises 1 to 57 nucleotides 3′ to the nucleotide edit to be incorporated to the target ATP7B gene sequence. In some embodiments, the editing template comprises 1 to 58 nucleotides 3′ to the nucleotide edit to be incorporated to the target ATP7B gene sequence. In some embodiments, the editing template comprises 1 to 59 nucleotides 3′ to the nucleotide edit to be incorporated to the target ATP7B gene sequence. In some embodiments, the editing template comprises 1 to 60 nucleotides 3′ to the nucleotide edit to be incorporated to the target ATP7B gene sequence. In some embodiments, the editing template comprises 1 to 61 nucleotides 3′ to the nucleotide edit to be incorporated to the target ATP7B gene sequence. In some embodiments, the editing template comprises 1 to 62 nucleotides 3′ to the nucleotide edit to be incorporated to the target ATP7B gene sequence. In some embodiments, the editing template comprises 1 to 63 nucleotides 3′ to the nucleotide edit to be incorporated to the target ATP7B gene sequence. In some embodiments, the editing template comprises 1 to 64 nucleotides 3′ to the nucleotide edit to be incorporated to the target ATP7B gene sequence. In some embodiments, the editing template comprises 1 to 65 nucleotides 3′ to the nucleotide edit to be incorporated to the target ATP7B gene sequence. In some embodiments, the editing template comprises 1 to 66 nucleotides 3′ to the nucleotide edit to be incorporated to the target ATP7B gene sequence. In some embodiments, the editing template comprises 1 to 67 nucleotides 3′ to the nucleotide edit to be incorporated to the target ATP7B gene sequence. In some embodiments, the editing template comprises 1 to 68 nucleotides 3′ to the nucleotide edit to be incorporated to the target ATP7B gene sequence. In some embodiments, the editing template comprises 1 to 69 nucleotides 3′ to the nucleotide edit to be incorporated to the target ATP7B gene sequence. In some embodiments, the editing template comprises 1 to 70 nucleotides 3′ to the nucleotide edit to be incorporated to the target ATP7B gene sequence. In some embodiments, the editing template comprises 1 to 71 nucleotides 3′ to the nucleotide edit to be incorporated to the target ATP7B gene sequence. In some embodiments, the editing template comprises 1 to 72 nucleotides 3′ to the nucleotide edit to be incorporated to the target ATP7B gene sequence. In some embodiments, the editing template comprises 1 to 73 nucleotides 3′ to the nucleotide edit to be incorporated to the target ATP7B gene sequence. In some embodiments, the editing template comprises 1 to 74 nucleotides 3′ to the nucleotide edit to be incorporated to the target ATP7B gene sequence. In some embodiments, the editing template comprises 1 to 75 nucleotides 3′ to the nucleotide edit to be incorporated to the target ATP7B gene sequence. In some embodiments, the editing template comprises 1 to 76 nucleotides 3′ to the nucleotide edit to be incorporated to the target ATP7B gene sequence. In some embodiments, the editing template comprises 1 to 77 nucleotides 3′ to the nucleotide edit to be incorporated to the target ATP7B gene sequence. In some embodiments, the editing template comprises 1 to 78 nucleotides 3′ to the nucleotide edit to be incorporated to the target ATP7B gene sequence. In some embodiments, the editing template comprises 2 to 80 nucleotides 3′ to the nucleotide edit to be incorporated to the target ATP7B gene sequence. In some embodiments, the editing template comprises 3 to 80 nucleotides 3′ to the nucleotide edit to be incorporated to the target ATP7B gene sequence. In some embodiments, the editing template comprises 4 to 80 nucleotides 3′ to the nucleotide edit to be incorporated to the target ATP7B gene sequence. In some embodiments, the editing template comprises 5 to 80 nucleotides 3′ to the nucleotide edit to be incorporated to the target ATP7B gene sequence. In some embodiments, the editing template comprises 6 to 80 nucleotides 3′ to the nucleotide edit to be incorporated to the target ATP7B gene sequence. In some embodiments, the editing template comprises 7 to 80 nucleotides 3′ to the nucleotide edit to be incorporated to the target ATP7B gene sequence. In some embodiments, the editing template comprises 8 to 80 nucleotides 3′ to the nucleotide edit to be incorporated to the target ATP7B gene sequence. In some embodiments, the editing template comprises 9 to 80 nucleotides 3′ to the nucleotide edit to be incorporated to the target ATP7B gene sequence. In some embodiments, the editing template comprises 10 to 80 nucleotides 3′ to the nucleotide edit to be incorporated to the target ATP7B gene sequence. In some embodiments, the editing template comprises 11 to 80 nucleotides 3′ to the nucleotide edit to be incorporated to the target ATP7B gene sequence. In some embodiments, the editing template comprises 12 to 80 nucleotides 3′ to the nucleotide edit to be incorporated to the target ATP7B gene sequence. In some embodiments, the editing template comprises 13 to 80 nucleotides 3′ to the nucleotide edit to be incorporated to the target ATP7B gene sequence. In some embodiments, the editing template comprises 14 to 80 nucleotides 3′ to the nucleotide edit to be incorporated to the target ATP7B gene sequence. In some embodiments, the editing template comprises 15 to 80 nucleotides 3′ to the nucleotide edit to be incorporated to the target ATP7B gene sequence. In some embodiments, the editing template comprises 16 to 80 nucleotides 3′ to the nucleotide edit to be incorporated to the target ATP7B gene sequence. In some embodiments, the editing template comprises 17 to 80 nucleotides 3′ to the nucleotide edit to be incorporated to the target ATP7B gene sequence. In some embodiments, the editing template comprises 18 to 80 nucleotides 3′ to the nucleotide edit to be incorporated to the target ATP7B gene sequence. In some embodiments, the editing template comprises 19 to 80 nucleotides 3′ to the nucleotide edit to be incorporated to the target ATP7B gene sequence. In some embodiments, the editing template comprises 20 to 80 nucleotides 3′ to the nucleotide edit to be incorporated to the target ATP7B gene sequence. In some embodiments, the editing template comprises 21 to 80 nucleotides 3′ to the nucleotide edit to be incorporated to the target ATP7B gene sequence. In some embodiments, the editing template comprises 22 to 80 nucleotides 3′ to the nucleotide edit to be incorporated to the target ATP7B gene sequence. In some embodiments, the editing template comprises 23 to 80 nucleotides 3′ to the nucleotide edit to be incorporated to the target ATP7B gene sequence. In some embodiments, the editing template comprises 24 to 80 nucleotides 3′ to the nucleotide edit to be incorporated to the target ATP7B gene sequence. In some embodiments, the editing template comprises 25 to 80 nucleotides 3′ to the nucleotide edit to be incorporated to the target ATP7B gene sequence. In some embodiments, the editing template comprises 26 to 80 nucleotides 3′ to the nucleotide edit to be incorporated to the target ATP7B gene sequence. In some embodiments, the editing template comprises 27 to 80 nucleotides 3′ to the nucleotide edit to be incorporated to the target ATP7B gene sequence. In some embodiments, the editing template comprises 28 to 80 nucleotides 3′ to the nucleotide edit to be incorporated to the target ATP7B gene sequence. In some embodiments, the editing template comprises 29 to 80 nucleotides 3′ to the nucleotide edit to be incorporated to the target ATP7B gene sequence. In some embodiments, the editing template comprises 30 to 80 nucleotides 3′ to the nucleotide edit to be incorporated to the target ATP7B gene sequence. In some embodiments, the editing template comprises 31 to 80 nucleotides 3′ to the nucleotide edit to be incorporated to the target ATP7B gene sequence. In some embodiments, the editing template comprises 32 to 80 nucleotides 3′ to the nucleotide edit to be incorporated to the target ATP7B gene sequence. In some embodiments, the editing template comprises 33 to 80 nucleotides 3′ to the nucleotide edit to be incorporated to the target ATP7B gene sequence. In some embodiments, the editing template comprises 34 to 80 nucleotides 3′ to the nucleotide edit to be incorporated to the target ATP7B gene sequence. In some embodiments, the editing template comprises 35 to 80 nucleotides 3′ to the nucleotide edit to be incorporated to the target ATP7B gene sequence. In some embodiments, the editing template comprises 36 to 80 nucleotides 3′ to the nucleotide edit to be incorporated to the target ATP7B gene sequence. In some embodiments, the editing template comprises 37 to 80 nucleotides 3′ to the nucleotide edit to be incorporated to the target ATP7B gene sequence. In some embodiments, the editing template comprises 38 to 80 nucleotides 3′ to the nucleotide edit to be incorporated to the target ATP7B gene sequence. In some embodiments, the editing template comprises 39 to 80 nucleotides 3′ to the nucleotide edit to be incorporated to the target ATP7B gene sequence. In some embodiments, the editing template comprises 40 to 80 nucleotides 3′ to the nucleotide edit to be incorporated to the target ATP7B gene sequence. In some embodiments, the editing template comprises 41 to 80 nucleotides 3′ to the nucleotide edit to be incorporated to the target ATP7B gene sequence. In some embodiments, the editing template comprises 42 to 80 nucleotides 3′ to the nucleotide edit to be incorporated to the target ATP7B gene sequence. In some embodiments, the editing template comprises 43 to 80 nucleotides 3′ to the nucleotide edit to be incorporated to the target ATP7B gene sequence. In some embodiments, the editing template comprises 44 to 80 nucleotides 3′ to the nucleotide edit to be incorporated to the target ATP7B gene sequence. In some embodiments, the editing template comprises 45 to 80 nucleotides 3′ to the nucleotide edit to be incorporated to the target ATP7B gene sequence. In some embodiments, the editing template comprises 46 to 80 nucleotides 3′ to the nucleotide edit to be incorporated to the target ATP7B gene sequence. In some embodiments, the editing template comprises 47 to 80 nucleotides 3′ to the nucleotide edit to be incorporated to the target ATP7B gene sequence. In some embodiments, the editing template comprises 48 to 80 nucleotides 3′ to the nucleotide edit to be incorporated to the target ATP7B gene sequence. In some embodiments, the editing template comprises 49 to 80 nucleotides 3′ to the nucleotide edit to be incorporated to the target ATP7B gene sequence. In some embodiments, the editing template comprises 50 to 80 nucleotides 3′ to the nucleotide edit to be incorporated to the target ATP7B gene sequence. In some embodiments, the editing template comprises 51 to 80 nucleotides 3′ to the nucleotide edit to be incorporated to the target ATP7B gene sequence. In some embodiments, the editing template comprises 52 to 80 nucleotides 3′ to the nucleotide edit to be incorporated to the target ATP7B gene sequence. In some embodiments, the editing template comprises 53 to 80 nucleotides 3′ to the nucleotide edit to be incorporated to the target ATP7B gene sequence. In some embodiments, the editing template comprises 54 to 80 nucleotides 3′ to the nucleotide edit to be incorporated to the target ATP7B gene sequence. In some embodiments, the editing template comprises 55 to 80 nucleotides 3′ to the nucleotide edit to be incorporated to the target ATP7B gene sequence. In some embodiments, the editing template comprises 56 to 80 nucleotides 3′ to the nucleotide edit to be incorporated to the target ATP7B gene sequence. In some embodiments, the editing template comprises 57 to 80 nucleotides 3′ to the nucleotide edit to be incorporated to the target ATP7B gene sequence. In some embodiments, the editing template comprises 58 to 80 nucleotides 3′ to the nucleotide edit to be incorporated to the target ATP7B gene sequence. In some embodiments, the editing template comprises 59 to 80 nucleotides 3′ to the nucleotide edit to be incorporated to the target ATP7B gene sequence. In some embodiments, the editing template comprises 60 to 80 nucleotides 3′ to the nucleotide edit to be incorporated to the target ATP7B gene sequence. In some embodiments, the editing template comprises 61 to 80 nucleotides 3′ to the nucleotide edit to be incorporated to the target ATP7B gene sequence. In some embodiments, the editing template comprises 62 to 80 nucleotides 3′ to the nucleotide edit to be incorporated to the target ATP7B gene sequence. In some embodiments, the editing template comprises 63 to 80 nucleotides 3′ to the nucleotide edit to be incorporated to the target ATP7B gene sequence. In some embodiments, the editing template comprises 64 to 80 nucleotides 3′ to the nucleotide edit to be incorporated to the target ATP7B gene sequence. In some embodiments, the editing template comprises 65 to 80 nucleotides 3′ to the nucleotide edit to be incorporated to the target ATP7B gene sequence. In some embodiments, the editing template comprises 66 to 80 nucleotides 3′ to the nucleotide edit to be incorporated to the target ATP7B gene sequence. In some embodiments, the editing template comprises 67 to 80 nucleotides 3′ to the nucleotide edit to be incorporated to the target ATP7B gene sequence. In some embodiments, the editing template comprises 68 to 80 nucleotides 3′ to the nucleotide edit to be incorporated to the target ATP7B gene sequence. In some embodiments, the editing template comprises 69 to 80 nucleotides 3′ to the nucleotide edit to be incorporated to the target ATP7B gene sequence. In some embodiments, the editing template comprises 70 to 80 nucleotides 3′ to the nucleotide edit to be incorporated to the target ATP7B gene sequence. In some embodiments, the editing template comprises 71 to 80 nucleotides 3′ to the nucleotide edit to be incorporated to the target ATP7B gene sequence. In some embodiments, the editing template comprises 72 to 80 nucleotides 3′ to the nucleotide edit to be incorporated to the target ATP7B gene sequence. In some embodiments, the editing template comprises 73 to 80 nucleotides 3′ to the nucleotide edit to be incorporated to the target ATP7B gene sequence. In some embodiments, the editing template comprises 74 to 80 nucleotides 3′ to the nucleotide edit to be incorporated to the target ATP7B gene sequence. In some embodiments, the editing template comprises 75 to 80 nucleotides 3′ to the nucleotide edit to be incorporated to the target ATP7B gene sequence. In some embodiments, the editing template comprises 76 to 80 nucleotides 3′ to the nucleotide edit to be incorporated to the target ATP7B gene sequence. In some embodiments, the editing template comprises 77 to 80 nucleotides 3′ to the nucleotide edit to be incorporated to the target ATP7B gene sequence. In some embodiments, the editing template comprises 78 to 80 nucleotides 3′ to the nucleotide edit to be incorporated to the target ATP7B gene sequence. In some embodiments, the editing template comprises 79 to 80 nucleotides 3′ to the nucleotide edit to be incorporated to the target ATP7B gene sequence.

In some embodiments, the editing template comprises 2 to 40 nucleotides 3′ to the nucleotide edit to be incorporated to the target ATP7B gene sequence. In some embodiments, the editing template comprises 2 to 38 nucleotides 3′ to the nucleotide edit to be incorporated to the target ATP7B gene sequence. In some embodiments, the editing template comprises 2 to 36 nucleotides 3′ to the nucleotide edit to be incorporated to the target ATP7B gene sequence. In some embodiments, the editing template comprises 2 to 34 nucleotides 3′ to the nucleotide edit to be incorporated to the target ATP7B gene sequence. In some embodiments, the editing template comprises 2 to 32 nucleotides 3′ to the nucleotide edit to be incorporated to the target ATP7B gene sequence. In some embodiments, the editing template comprises 4 to 30 nucleotides 3′ to the nucleotide edit to be incorporated to the target ATP7B gene sequence. In some embodiments, the editing template comprises 2 to 25 nucleotides 3′ to the nucleotide edit to be incorporated to the target ATP7B gene sequence. In some embodiments, the editing template comprises 2 to 20 nucleotides 3′ to the nucleotide edit to be incorporated to the target ATP7B gene sequence. In some embodiments, the editing template comprises 2 to 15 nucleotides 3′ to the nucleotide edit to be incorporated to the target ATP7B gene sequence. In some embodiments, the editing template comprises 2 to 10 nucleotides 3′ to the nucleotide edit to be incorporated to the target ATP7B gene sequence. In some embodiments, the editing template comprises 2 to 5 nucleotides 3′ to the nucleotide edit to be incorporated to the target ATP7B gene sequence. In some embodiments, the editing template comprises 4 to 25 nucleotides 3′ to the nucleotide edit to be incorporated to the target ATP7B gene sequence. In some embodiments, the editing template comprises 4 to 20 nucleotides 3′ to the nucleotide edit to be incorporated to the target ATP7B gene sequence. In some embodiments, the editing template comprises 4 to 25 nucleotides 3′ to the nucleotide edit to be incorporated to the target ATP7B gene sequence. In some embodiments, the editing template comprises 4 to 15 nucleotides 3′ to the nucleotide edit to be incorporated to the target ATP7B gene sequence. In some embodiments, the editing template comprises 4 to 10 nucleotides 3′ to the nucleotide edit to be incorporated to the target ATP7B gene sequence. In some embodiments, the editing template comprises 10 to 15 nucleotides 3′ to the nucleotide edit to be incorporated to the target ATP7B gene sequence. In some embodiments, the editing template comprises 10 to 20 nucleotides 3′ to the nucleotide edit to be incorporated to the target ATP7B gene sequence. In some embodiments, the editing template comprises 10 to 30 nucleotides 3′ to the nucleotide edit to be incorporated to the target ATP7B gene sequence. In some embodiments, the editing template comprises 4 to 30 nucleotides 5′ to the nucleotide edit to be incorporated to the target ATP7B gene sequence. In some embodiments, the editing template comprises 4 to 25 nucleotides 5′ to the nucleotide edit to be incorporated to the target ATP7B gene sequence. In some embodiments, the editing template comprises 4 to 20 nucleotides 5′ to the nucleotide edit to be incorporated to the target ATP7B gene sequence.

In some embodiments, the length of the editing template is at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, or 80 nucleotides longer than the nick to edit distance. In some embodiments, for example, the nick to edit distance is 8 nucleotides, and the editing template is 10 to 15, 10 to 20, 10 to 25, 10 to 30, 10 to 35, 10 to 40, 10 to 45, 10 to 50, 10 to 55, 10 to 60, 10 to 65, 10 to 70, 10 to 75, or 10 to 80 nucleotides in length. In some embodiments, the nick to edit distance is 22 nucleotides, and the editing template is 24 to 28, 24 to 30, 24 to 32, 24 to 34, 24 to 36, 24 to 37, 24 to 38, 24 to 40, 24 to 45, 24 to 50, 24 to 55, 24 to 60, 24 to 65, 24 to 70, 24 to 75, 24 to 80, 24 to 85, 24 to 90, 24 to 95, 24 to 100, 24 to 105, 24 to 100, 24 to 105, or 24 to 110 nucleotides in length.

In some embodiments, the editing template comprises an adenine at the first nucleobase position (e.g., for a PEgRNA following 5′-spacer-gRNA core-RTT-PBS-3′ orientation, the 5′ most nucleobase is the “first base”). In some embodiments, the editing template comprises a guanine at the first nucleobase position (e.g., for a PEgRNA following 5′-spacer-gRNA core-RTT-PBS-3′ orientation, the 5′ most nucleobase is the “first base”). In some embodiments, the editing template comprises an uracil at the first nucleobase position (e.g., for a PEgRNA following 5′-spacer-gRNA core-RTT-PBS-3′ orientation, the 5′ most nucleobase is the “first base”). In some embodiments, the editing template comprises a cytosine at the first nucleobase position (e.g., for a PEgRNA following 5′-spacer-gRNA core-RTT-PBS-3′ orientation, the 5′ most nucleobase is the “first base”). In some embodiments, the editing template does not comprise a cytosine at the first nucleobase position (e.g., for a PEgRNA following 5′-spacer-gRNA core-RT T-PBS-3′ orientation, the 5′ most nucleobase is the “first base”).

The editing template of a PEgRNA may encode a new single stranded DNA (e.g. by reverse transcription) to replace a editing target sequence in the target gene. In some embodiments, the editing target sequence in the edit strand of the target gene is replaced by the newly synthesized strand, and the nucleotide edit(s) are incorporated in the region of the target gene. In some embodiments, the target gene is an ATP7B gene. In some embodiments, the editing template of the PEgRNA encodes a newly synthesized single stranded DNA that comprises a wild type APT7B gene sequence. In some embodiments, the newly synthesized DNA strand replaces the editing target sequence in the target ATP7B gene, wherein the editing target sequence (or the endogenous sequence complementary to the editing target sequence on the target strand of the ATP7B gene) comprises a mutation compared to a wild type ATP7B gene. In some embodiments, the mutation is associated with Wilson's disease.

In some embodiments, the editing target sequence comprises a mutation in exon 1, exon 2, exon 3, exon 4, exon 5, exon 6, exon 7, exon 8, exon 9, exon 10, exon 11, exon 12, exon 13, exon 14, exon 15, exon 16, exon 17, exon 18, exon 19, exon 20, or exon 21 of the ATP7B gene as compared to a wild type ATP7B gene. In some embodiments, the editing target sequence comprises a mutation in exon 8, exon 13, exon 14, exon 15, or exon 17 of the ATP7B gene as compared to a wild type ATP7B gene. In some embodiments, the editing target sequence comprises a mutation in exon 14 of the ATP7B gene as compared to a wild type ATP7B gene. In some embodiments, the editing target sequence comprises a mutation in exon 3 of the ATP7B gene as compared to a wild type ATP7B gene. In some embodiments, the editing target sequence comprises a mutation that is located in exon 8 of the ATP7B gene as compared to a wild type ATP7B gene. In some embodiments, the mutation is not a c.1288dup duplication. In some embodiments, the editing target sequence comprises a mutation that is located between positions 51932669 and 52012130 of human chromosome 13 as set forth in human genome assembly consortium Human build 38 (GRCh38), GenBank accession GCA_000001405.15. In some embodiments, the editing target sequence comprises a mutation that is located between positions 51944045 and 51944245 of human chromosome 13 as set forth in human genome assembly consortium Human build 38 (GRCh38), GenBank accession GCA_000001405.15. In some embodiments, the editing target sequence comprises a mutation that encodes an amino acid substitution H1069Q relative to a wild type ATP7B polypeptide set forth in SEQ ID NO: 5861. In some embodiments, the editing target sequence comprises a C>A mutation at position 51944145 in human chromosome 13 as set forth in human genome assembly consortium Human build 38 (GRCh38), GenBank accession GCA_000001405.15. As used herein, unless otherwise noted, reference to positions in human genome is as set forth in human genome assembly consortium Human build 38 (GRCh38), GenBank accession GCA_000001405.15.

A guide RNA core (also referred to herein as the gRNA core, gRNA scaffold, or gRNA backbone sequence) of a PEgRNA may contain a polynucleotide sequence that binds to a DNA binding domain (e.g., Cas9) of a prime editor. The gRNA core may interact with a prime editor as described herein, for example, by association with a DNA binding domain, such as a DNA nickase of the prime editor.

One of skill in the art will recognize that different prime editors having different DNA binding domains from different DNA binding proteins may require different gRNA core sequences specific to the DNA binding protein. In some embodiments, the gRNA core is capable of binding to a Cas9-based prime editor. In some embodiments, the gRNA core is capable of binding to a Cpf1-based prime editor. In some embodiments, the gRNA core is capable of binding to a Cas12b-based prime editor.

In some embodiments, the gRNA core comprises regions and secondary structures involved in binding with specific CRISPR Cas proteins. For example, in a Cas9 based prime editing system, the gRNA core of a PEgRNA may comprise one or more regions of a base paired “lower stem” adjacent to the spacer sequence and a base paired “upper stem” following the lower stem, where the lower stem and upper stem may be connected by a “bulge” comprising unpaired RNAs. The gRNA core may further comprise a “nexus” distal from the spacer sequence, followed by a hairpin structure, e.g., at the 3′ end, as exemplified in FIG. 4. In some embodiments, the gRNA core comprises modified nucleotides as compared to a wild type gRNA core in the lower stem, upper stem, and/or the hairpin. For example, nucleotides in the lower stem, upper stem, an/or the hairpin regions may be modified, deleted, or replaced. In some embodiments. RNA nucleotides in the lower stem, upper stem, an/or the hairpin regions may be replaced with one or more DNA sequences. In some embodiments, the gRNA core comprises unmodified or wild type RNA sequences in the nexus and/or the bulge regions. In some embodiments, the gRNA core does not include long stretches of A-T pairs, for example, a GUUUU-AAAAC pairing element.

In some embodiments, a prime editing system comprises a prime editor and a PEgRNA, wherein the prime editor comprises a SpCas9 nickase or a variant thereof, and the gRNA core of the PEgRNA comprises the sequence:

(SEQ ID NO: 5857)
GUUUUAGAGCUAGAAAUAGCAAGUUAAAAUAAGGCUAGUCCGUUAUCAAC
UUGAAAAAGUGGCACCGAGUCGGUGC;
(SEQ ID NO: 5858)
GUUUGAGAGCUAGAAAUAGCAAGUUUAAAUAAGGCUAGUCCGUUAUCAAC
UUGAAAAAGUGGGACCGAGUCGGUCC,
or
(SEQ ID NO: 5859)
GUUUAAGAGCUAUGCUGGAAACAGCAUAGCAAGUUUAAAUAAGGCUAGUC
CGUUAUCAACUUGAAAAAGUGGCACCGAGUCGGUGC.

In some embodiments, the gRNA core comprises the sequence

(SEQ ID NO: 5857)
GUUUUAGAGCUAGAAAUAGCAAGUUAAAAUAAGGCUAGUCCGUUAUCAAC
UUGAAAAAGUGGCACCGAGUCGGUGC.

Any gRNA core sequences known in the art are also contemplated in the prime editing compositions described herein.

A PEgRNA may also comprise optional modifiers, e.g., 3′ end modifier region and/or an 5′ end modifier region. In some embodiments, a PEgRNA comprises at least one nucleotide that is not part of a spacer, a gRNA core, or an extension arm. The optional sequence modifiers could be positioned within or between any of the other regions shown, and not limited to being located at the 3′ and 5′ ends. In certain embodiments, the PEgRNA comprises secondary RNA structure, such as, but not limited to, aptamers, hairpins, stem/loops, toeloops, and/or RNA-binding protein recruitment domains (e.g., the MS2 aptamer which recruits and binds to the MS2cp protein). In some embodiments, a PEgRNA comprises a short stretch of uracil at the 5′ end or the 3′ end. For example, in some embodiments, a PEgRNA comprising a 3′ extension arm comprises a “UUU” sequence at the 3′ end of the extension arm. In some embodiments, a PEgRNA comprises a toeloop sequence at the 3′ end. In some embodiments, the PEgRNA comprises a 3′ extension arm and a toeloop sequence at the 3′ end of the extension arm. In some embodiments, the PEgRNA comprises a 5′ extension arm and a toeloop sequence at the 5′ end of the extension arm. In some embodiments, the PEgRNA comprises a toeloop element having the sequence 5′-GAAANNNNN-3′, wherein N is any nucleobase. In some embodiments, the secondary RNA structure is positioned within the spacer. In some embodiments, the secondary structure is positioned within the extension arm. In some embodiments, the secondary structure is positioned within the gRNA core. In some embodiments, the secondary structure is positioned between the spacer and the gRNA core, between the gRNA core and the extension arm, or between the spacer and the extension arm. In some embodiments, the secondary structure is positioned between the PBS and the editing template. In some embodiments the secondary structure is positioned at the 3′ end or at the 5′ end of the PEgRNA. In some embodiments, the PEgRNA comprises a transcriptional termination signal at the 3′ end of the PEgRNA. In addition to secondary RNA structures, the PEgRNA may comprise a chemical linker or a poly(N) linker or tail, where “N” can be any nucleobase. In some embodiments, the chemical linker may function to prevent reverse transcription of the gRNA core.

In some embodiments, a prime editing system or composition further comprises a nick guide polynucleotide, such as a nick guide RNA (ngRNA). Without wishing to be bound by any particular theory, the non-edit strand of a double stranded target DNA in the target gene may be nicked by a CRISPR-Cas nickase directed by an ngRNA. In some embodiments, the nick on the non-edit strand directs endogenous DNA repair machinery to use the edit strand as a template for repair of the non-edit strand, which may increase efficiency of prime editing. In some embodiments, the non-edit strand is nicked by a prime editor localized to the non-edit strand by the ngRNA. Accordingly, also provided herein are PEgRNA systems comprising at least one PEgRNA and at least one ngRNA.

In some embodiments, the ngRNA is a guide RNA which contains a variable spacer sequence and a guide RNA scaffold or core region that interacts with the DNA binding domain, e.g. Cas9 of the prime editor. In some embodiments, the ngRNA comprises a spacer sequence (referred to herein as an ng spacer, or a second spacer) that is substantially complementary to a second search target sequence (or ng search target sequence), which is located on the edit strand, or the non-target strand. Thus, in some embodiments, the ng search target sequence recognized by the ng spacer and the search target sequence recognized by the spacer sequence of the PEgRNA are on opposite strands of the double stranded target DNA of target gene, e.g., the ATP7B gene. A prime editing system or complex comprising a ngRNA may be referred to as a “PE3” prime editing system or PE3 prime editing complex.

In some embodiments, the ng search target sequence is located on the non-target strand, within 10 base pairs to 100 base pairs of an intended nucleotide edit incorporated by the PEgRNA on the edit strand. In some embodiments, the ng target search target sequence is within 10 bp, 20 bp, 30 bp, 40 bp, 50 bp, 60 bp, 70 bp, 80 bp, 90 bp, 91 bp, 92 bp, 93 bp, 94 bp, 95 bp, 96 bp, 97 bp, 98 bp, 99 bp, or 100 bp of an intended nucleotide edit incorporated by the PEgRNA on the edit strand. In some embodiments, the 5′ ends of the ng search target sequence and the PEgRNA search target sequence are within 1, 2, 3, 4, 5, 6, 7, 8, 9, or 10 bp apart from each other. In some embodiments, the 5′ ends of the ng search target sequence and the PEgRNA search target sequence are within 10 bp, 20 bp, 30 bp, 40 bp, 50 bp, 60 bp, 70 bp, 80 bp, 90 bp, 91 bp, 92 bp, 93 bp, 94 bp, 95 bp, 96 bp, 97 bp, 98 bp, 99 bp, or 100 bp apart from each other.

In some embodiments, an ng spacer sequence is complementary to, and may hybridize with the second search target sequence only after an intended nucleotide edit has been incorporated on the edit strand, by the editing template of a PEgRNA. Such a prime editing system maybe referred to as a “PE3b” prime editing system or composition. In some embodiments, the ngRNA comprises a spacer sequence that matches only the edit strand after incorporation of the nucleotide edits, but not the endogenous target gene sequence on the edit strand. Accordingly, in some embodiments, an intended nucleotide edit is incorporated within the ng search target sequence. In some embodiments, the intended nucleotide edit is incorporated within about 1-10 nucleotides of the position corresponding to the PAM of the ng search target sequence.

Exemplary combinations of PEgRNA components, e.g., spacer, PBS, and RTT, as well as combinations of each PEgRNA and corresponding ngRNA(s) are provided in Tables 6-12 and 15-32. Tables 6-12, 15-32 each contains two columns, the left column lists the respective PEgRNA components, and the right column is the corresponding sequence identifiers. Each of the PEgRNA components in Tables 6-12, 15-32 is listed consecutively and should be read from left to right, continuously.

TABLE 6

| Sequence region | SEQ ID |
|---|---|
| pegRNA Spacer | 1 |
| PBS | 2 |
| PBS | 3 |
| PBS | 4 |
| PBS | 5 |
| PBS | 6 |
| PBS | 7 |
| PBS | 8 |
| PBS | 9 |
| PBS | 10 |
| PBS | 11 |
| PBS | 12 |
| RTT | 13 |
| RTT | 14 |
| RTT | 15 |
| RTT | 16 |
| RTT | 17 |
| ngRNA Spacer | 18 |
| ngRNA Spacer | 19 |
| ngRNA Spacer | 20 |
| ngRNA Spacer | 21 |
| ngRNA Spacer | 22 |
| ngRNA Spacer | 23 |
| ngRNA Spacer | 24 |
| ngRNA Spacer | 25 |
| ngRNA Spacer | 26 |
| ngRNA Spacer | 27 |
| ngRNA Spacer | 28 |
| ngRNA Spacer | 29 |
| ngRNA Spacer | 30 |
| ngRNA Spacer | 31 |
| ngRNA Spacer | 32 |
| ngRNA Spacer | 33 |
| ngRNA Spacer | 34 |
| ngRNA Spacer | 35 |
| ngRNA Spacer | 36 |
| ngRNA Spacer | 37 |
| ngRNA Spacer | 38 |
| ngRNA Spacer | 39 |
| ngRNA Spacer | 40 |
| ngRNA Spacer | 41 |
| ngRNA Spacer | 42 |
| ngRNA Spacer | 43 |
| ngRNA Spacer | 44 |
| ngRNA Spacer | 45 |
| ngRNA Spacer | 46 |
| ngRNA Spacer | 47 |
| ngRNA Spacer | 48 |
| ngRNA Spacer | 49 |
| ngRNA Spacer | 50 |
| ngRNA Spacer | 51 |
| ngRNA Spacer | 52 |
| ngRNA Spacer | 53 |
| ngRNA Spacer | 54 |
| ngRNA Spacer | 55 |
| ngRNA Spacer | 56 |
| ngRNA Spacer | 57 |
| ngRNA Spacer | 58 |
| ngRNA Spacer | 59 |
| ngRNA Spacer | 60 |
| ngRNA Spacer | 61 |
| ngRNA Spacer | 62 |
| ngRNA Spacer | 63 |
| ngRNA Spacer | 64 |
| ngRNA Spacer | 65 |
| ngRNA Spacer | 66 |
| ngRNA Spacer | 67 |
| ngRNA Spacer | 68 |
| ngRNA Spacer | 69 |
| ngRNA Spacer | 70 |
| ngRNA Spacer | 71 |
| ngRNA Spacer | 72 |
| pegRNA | 73 |
| pegRNA | 74 |
| pegRNA | 75 |
| pegRNA | 76 |
| pegRNA | 77 |
| pegRNA | 78 |
| pegRNA | 79 |
| pegRNA | 80 |
| pegRNA | 81 |
| pegRNA | 82 |
| pegRNA | 83 |
| pegRNA | 84 |
| pegRNA | 85 |
| pegRNA | 86 |
| pegRNA | 87 |
| pegRNA | 88 |
| pegRNA | 89 |
| pegRNA | 90 |
| pegRNA | 91 |
| pegRNA | 92 |
| pegRNA | 93 |
| pegRNA | 94 |
| pegRNA | 95 |
| pegRNA | 96 |
| pegRNA | 97 |
| pegRNA | 98 |
| pegRNA | 99 |
| pegRNA | 100 |
| pegRNA | 101 |
| pegRNA | 102 |
| pegRNA | 103 |
| pegRNA | 104 |
| pegRNA | 105 |
| pegRNA | 106 |
| pegRNA | 107 |
| pegRNA | 108 |
| pegRNA | 109 |
| pegRNA | 110 |
| pegRNA | 111 |
| pegRNA | 112 |
| pegRNA | 113 |
| pegRNA | 114 |
| pegRNA | 115 |
| pegRNA | 116 |
| pegRNA | 117 |
| pegRNA | 118 |
| pegRNA | 119 |
| pegRNA | 120 |
| pegRNA | 121 |
| pegRNA | 122 |
| pegRNA | 123 |
| pegRNA | 124 |
| pegRNA | 125 |
| pegRNA | 126 |
| pegRNA | 127 |
| pegRNA | 128 |
| pegRNA | 129 |
| pegRNA | 130 |
| pegRNA | 131 |
| pegRNA | 132 |
| pegRNA | 133 |
| pegRNA | 134 |
| pegRNA | 135 |
| pegRNA | 136 |
| pegRNA | 137 |
| pegRNA | 138 |
| pegRNA | 139 |
| pegRNA | 140 |
| pegRNA | 141 |
| pegRNA | 142 |
| pegRNA | 143 |
| pegRNA | 144 |
| pegRNA | 145 |
| pegRNA | 146 |
| pegRNA | 147 |
| pegRNA | 148 |
| pegRNA | 149 |
| pegRNA | 150 |
| pegRNA | 151 |
| pegRNA | 152 |
| ngRNA | 153 |
| ngRNA | 154 |
| ngRNA | 155 |
| ngRNA | 156 |
| ngRNA | 157 |
| ngRNA | 158 |
| ngRNA | 159 |
| ngRNA | 160 |
| ngRNA | 161 |
| ngRNA | 162 |
| ngRNA | 163 |
| ngRNA | 164 |
| ngRNA | 165 |
| ngRNA | 166 |
| ngRNA | 167 |
| ngRNA | 168 |
| ngRNA | 169 |
| ngRNA | 170 |
| ngRNA | 171 |
| ngRNA | 172 |
| ngRNA | 173 |
| ngRNA | 174 |
| ngRNA | 175 |
| ngRNA | 176 |
| ngRNA | 177 |
| ngRNA | 178 |
| ngRNA | 179 |
| ngRNA | 180 |
| ngRNA | 181 |

TABLE 7

| Sequence region | SEQ ID |
|---|---|
| pegRNA Spacer | 182 |
| PBS | 183 |
| PBS | 184 |
| PBS | 185 |
| PBS | 186 |
| PBS | 187 |
| PBS | 188 |
| PBS | 189 |
| PBS | 190 |
| PBS | 191 |
| PBS | 192 |
| PBS | 193 |
| RTT | 194 |
| RTT | 195 |
| RTT | 196 |
| RTT | 197 |
| RTT | 198 |
| ngRNA Spacer | 42 |
| ngRNA Spacer | 22 |
| ngRNA Spacer | 55 |
| ngRNA Spacer | 54 |
| ngRNA Spacer | 199 |
| ngRNA Spacer | 62 |
| ngRNA Spacer | 34 |
| ngRNA Spacer | 72 |
| ngRNA Spacer | 63 |
| ngRNA Spacer | 43 |
| ngRNA Spacer | 66 |
| ngRNA Spacer | 200 |
| ngRNA Spacer | 28 |
| ngRNA Spacer | 30 |
| ngRNA Spacer | 20 |
| ngRNA Spacer | 50 |
| ngRNA Spacer | 35 |
| ngRNA Spacer | 27 |
| ngRNA Spacer | 31 |
| ngRNA Spacer | 48 |
| ngRNA Spacer | 61 |
| ngRNA Spacer | 69 |
| ngRNA Spacer | 26 |
| ngRNA Spacer | 45 |
| ngRNA Spacer | 37 |
| ngRNA Spacer | 33 |
| ngRNA Spacer | 60 |
| ngRNA Spacer | 58 |
| ngRNA Spacer | 68 |
| ngRNA Spacer | 70 |
| ngRNA Spacer | 25 |
| ngRNA Spacer | 46 |
| ngRNA Spacer | 24 |
| ngRNA Spacer | 38 |
| ngRNA Spacer | 56 |
| ngRNA Spacer | 201 |
| ngRNA Spacer | 36 |
| ngRNA Spacer | 44 |
| ngRNA Spacer | 57 |
| ngRNA Spacer | 23 |
| ngRNA Spacer | 21 |
| ngRNA Spacer | 202 |
| ngRNA Spacer | 41 |
| ngRNA Spacer | 29 |
| ngRNA Spacer | 203 |
| ngRNA Spacer | 71 |
| ngRNA Spacer | 32 |
| ngRNA Spacer | 67 |
| ngRNA Spacer | 19 |
| ngRNA Spacer | 39 |
| ngRNA Spacer | 51 |
| ngRNA Spacer | 47 |
| ngRNA Spacer | 40 |
| ngRNA Spacer | 204 |
| ngRNA Spacer | 64 |
| ngRNA Spacer | 205 |
| ngRNA Spacer | 53 |
| ngRNA Spacer | 206 |
| ngRNA Spacer | 65 |
| ngRNA Spacer | 207 |
| ngRNA Spacer | 18 |
| ngRNA Spacer | 52 |
| ngRNA Spacer | 208 |
| ngRNA Spacer | 59 |
| ngRNA Spacer | 49 |
| ngRNA Spacer | 209 |
| pegRNA | 210 |
| pegRNA | 211 |
| pegRNA | 212 |
| pegRNA | 213 |
| pegRNA | 214 |
| pegRNA | 215 |
| pegRNA | 216 |
| pegRNA | 217 |
| pegRNA | 218 |
| pegRNA | 219 |
| pegRNA | 220 |
| pegRNA | 221 |
| pegRNA | 222 |
| pegRNA | 223 |
| pegRNA | 224 |
| pegRNA | 225 |
| pegRNA | 226 |
| pegRNA | 227 |
| pegRNA | 228 |
| pegRNA | 229 |
| pegRNA | 230 |
| pegRNA | 231 |
| pegRNA | 232 |
| pegRNA | 233 |
| pegRNA | 234 |
| pegRNA | 235 |
| pegRNA | 236 |
| pegRNA | 237 |
| pegRNA | 238 |
| pegRNA | 239 |
| pegRNA | 240 |
| pegRNA | 241 |
| pegRNA | 242 |
| pegRNA | 243 |
| pegRNA | 244 |
| pegRNA | 245 |
| pegRNA | 246 |
| pegRNA | 247 |
| pegRNA | 248 |
| pegRNA | 249 |
| pegRNA | 250 |
| pegRNA | 251 |
| pegRNA | 252 |
| pegRNA | 253 |
| pegRNA | 254 |
| pegRNA | 255 |
| pegRNA | 256 |
| pegRNA | 257 |
| pegRNA | 258 |
| pegRNA | 259 |
| pegRNA | 260 |
| pegRNA | 261 |
| pegRNA | 262 |
| pegRNA | 263 |
| pegRNA | 265 |
| pegRNA | 266 |
| pegRNA | 267 |
| pegRNA | 268 |
| pegRNA | 269 |
| pegRNA | 270 |
| pegRNA | 271 |
| pegRNA | 272 |
| pegRNA | 273 |
| pegRNA | 274 |
| pegRNA | 275 |
| pegRNA | 276 |
| pegRNA | 277 |
| pegRNA | 278 |
| pegRNA | 279 |
| pegRNA | 280 |
| pegRNA | 281 |
| pegRNA | 282 |
| pegRNA | 283 |
| pegRNA | 284 |
| pegRNA | 285 |
| pegRNA | 286 |
| pegRNA | 287 |
| pegRNA | 288 |
| pegRNA | 289 |
| ngRNA | 153 |
| ngRNA | 156 |
| ngRNA | 163 |
| ngRNA | 162 |
| ngRNA | 165 |
| ngRNA | 169 |
| ngRNA | 158 |
| ngRNA | 164 |
| ngRNA | 290 |
| ngRNA | 159 |
| ngRNA | 155 |
| ngRNA | 161 |
| ngRNA | 168 |
| ngRNA | 167 |
| ngRNA | 157 |
| ngRNA | 291 |
| ngRNA | 166 |
| ngRNA | 154 |
| ngRNA | 160 |
| ngRNA | 173 |
| ngRNA | 175 |
| ngRNA | 177 |
| ngRNA | 170 |
| ngRNA | 292 |
| ngRNA | 176 |
| ngRNA | 172 |
| ngRNA | 179 |
| ngRNA | 293 |
| ngRNA | 178 |
| ngRNA | 180 |
| ngRNA | 171 |
| ngRNA | 181 |
| ngRNA | 174 |

TABLE 8

| Sequence region | SEQ ID |
|---|---|
| pegRNA Spacer | 294 |
| PBS | 295 |
| PBS | 296 |
| PBS | 297 |
| PBS | 298 |
| PBS | 299 |
| PBS | 300 |
| PBS | 301 |
| PBS | 302 |
| PBS | 303 |
| PBS | 304 |
| PBS | 305 |
| RTT | 306 |
| RTT | 307 |
| RTT | 308 |
| RTT | 309 |
| RTT | 310 |
| RTT | 311 |
| RTT | 312 |
| RTT | 313 |
| RTT | 314 |
| RTT | 315 |
| RTT | 316 |
| RTT | 317 |
| RTT | 318 |
| RTT | 319 |
| RTT | 320 |
| RTT | 321 |
| RTT | 322 |
| RTT | 323 |
| RTT | 324 |
| RTT | 325 |
| RTT | 326 |
| RTT | 327 |
| RTT | 328 |
| RTT | 329 |
| RTT | 330 |
| RTT | 331 |
| RTT | 332 |
| RTT | 333 |
| RTT | 334 |
| RTT | 335 |
| RTT | 336 |
| ngRNA Spacer | 69 |
| ngRNA Spacer | 63 |
| ngRNA Spacer | 62 |
| ngRNA Spacer | 66 |
| ngRNA Spacer | 61 |
| ngRNA Spacer | 71 |
| ngRNA Spacer | 67 |
| ngRNA Spacer | 53 |
| ngRNA Spacer | 59 |
| ngRNA Spacer | 24 |
| ngRNA Spacer | 52 |
| ngRNA Spacer | 39 |
| ngRNA Spacer | 40 |
| ngRNA Spacer | 20 |
| ngRNA Spacer | 200 |
| ngRNA Spacer | 46 |
| ngRNA Spacer | 35 |
| ngRNA Spacer | 201 |
| ngRNA Spacer | 206 |
| ngRNA Spacer | 36 |
| ngRNA Spacer | 70 |
| ngRNA Spacer | 28 |
| ngRNA Spacer | 45 |
| ngRNA Spacer | 47 |
| ngRNA Spacer | 27 |
| ngRNA Spacer | 29 |
| ngRNA Spacer | 44 |
| ngRNA Spacer | 204 |
| ngRNA Spacer | 19 |
| ngRNA Spacer | 202 |
| ngRNA Spacer | 65 |
| ngRNA Spacer | 207 |
| ngRNA Spacer | 64 |
| ngRNA Spacer | 37 |
| ngRNA Spacer | 60 |
| ngRNA Spacer | 205 |
| ngRNA Spacer | 57 |
| ngRNA Spacer | 209 |
| ngRNA Spacer | 18 |
| ngRNA Spacer | 51 |
| ngRNA Spacer | 49 |
| ngRNA Spacer | 42 |
| ngRNA Spacer | 56 |
| ngRNA Spacer | 208 |
| ngRNA Spacer | 33 |
| ngRNA Spacer | 199 |
| ngRNA Spacer | 54 |
| ngRNA Spacer | 43 |
| ngRNA Spacer | 55 |
| ngRNA Spacer | 58 |
| ngRNA Spacer | 41 |
| ngRNA Spacer | 25 |
| ngRNA Spacer | 26 |
| ngRNA Spacer | 34 |
| ngRNA Spacer | 38 |
| ngRNA Spacer | 68 |
| ngRNA Spacer | 72 |
| ngRNA Spacer | 23 |
| ngRNA Spacer | 203 |
| ngRNA Spacer | 32 |
| ngRNA Spacer | 50 |
| ngRNA Spacer | 337 |
| ngRNA Spacer | 22 |
| ngRNA Spacer | 48 |
| ngRNA Spacer | 31 |
| ngRNA Spacer | 21 |
| pegRNA | 338 |
| pegRNA | 339 |
| pegRNA | 340 |
| pegRNA | 341 |
| pegRNA | 342 |
| pegRNA | 343 |
| pegRNA | 344 |
| pegRNA | 345 |
| pegRNA | 346 |
| pegRNA | 347 |
| pegRNA | 348 |
| pegRNA | 349 |
| pegRNA | 349 |
| pegRNA | 351 |
| pegRNA | 352 |
| pegRNA | 353 |
| pegRNA | 354 |
| pegRNA | 355 |
| pegRNA | 356 |
| pegRNA | 357 |
| pegRNA | 356 |
| pegRNA | 358 |
| pegRNA | 359 |
| pegRNA | 360 |
| pegRNA | 361 |
| pegRNA | 362 |
| pegRNA | 363 |
| pegRNA | 364 |
| pegRNA | 365 |
| pegRNA | 366 |
| pegRNA | 366 |
| pegRNA | 365 |
| pegRNA | 367 |
| pegRNA | 368 |
| pegRNA | 369 |
| pegRNA | 370 |
| pegRNA | 371 |
| pegRNA | 372 |
| pegRNA | 373 |
| pegRNA | 374 |
| pegRNA | 375 |
| pegRNA | 375 |
| pegRNA | 376 |
| pegRNA | 377 |
| pegRNA | 378 |
| pegRNA | 379 |
| pegRNA | 380 |
| pegRNA | 381 |
| pegRNA | 382 |
| pegRNA | 383 |
| pegRNA | 384 |
| pegRNA | 385 |
| pegRNA | 386 |
| pegRNA | 387 |
| pegRNA | 388 |
| pegRNA | 389 |
| pegRNA | 389 |
| pegRNA | 390 |
| pegRNA | 390 |
| pegRNA | 391 |
| pegRNA | 392 |
| pegRNA | 393 |
| pegRNA | 394 |
| pegRNA | 395 |
| pegRNA | 396 |
| pegRNA | 397 |
| pegRNA | 398 |
| pegRNA | 399 |
| pegRNA | 400 |
| pegRNA | 401 |
| pegRNA | 402 |
| pegRNA | 403 |
| pegRNA | 404 |
| pegRNA | 405 |
| pegRNA | 406 |
| pegRNA | 407 |
| pegRNA | 408 |
| pegRNA | 409 |
| pegRNA | 410 |
| pegRNA | 411 |
| pegRNA | 412 |
| pegRNA | 413 |
| pegRNA | 414 |
| pegRNA | 415 |
| pegRNA | 416 |
| pegRNA | 417 |
| pegRNA | 417 |
| pegRNA | 418 |
| pegRNA | 419 |
| pegRNA | 420 |
| pegRNA | 421 |
| pegRNA | 422 |
| pegRNA | 423 |
| pegRNA | 424 |
| pegRNA | 425 |
| pegRNA | 426 |
| pegRNA | 427 |
| pegRNA | 428 |
| pegRNA | 429 |
| pegRNA | 430 |
| pegRNA | 431 |
| pegRNA | 432 |
| pegRNA | 433 |
| pegRNA | 434 |
| pegRNA | 435 |
| pegRNA | 436 |
| pegRNA | 437 |
| pegRNA | 438 |
| pegRNA | 439 |
| pegRNA | 440 |
| pegRNA | 441 |
| pegRNA | 442 |
| pegRNA | 443 |
| pegRNA | 444 |
| pegRNA | 445 |
| pegRNA | 446 |
| pegRNA | 447 |
| pegRNA | 448 |
| pegRNA | 449 |
| pegRNA | 450 |
| pegRNA | 451 |
| pegRNA | 452 |
| pegRNA | 453 |
| pegRNA | 454 |
| pegRNA | 455 |
| pegRNA | 456 |
| pegRNA | 457 |
| pegRNA | 458 |
| pegRNA | 459 |
| pegRNA | 460 |
| pegRNA | 461 |
| pegRNA | 462 |
| pegRNA | 463 |
| pegRNA | 464 |
| pegRNA | 465 |
| pegRNA | 466 |
| pegRNA | 467 |
| pegRNA | 468 |
| pegRNA | 469 |
| pegRNA | 470 |
| pegRNA | 471 |
| pegRNA | 472 |
| pegRNA | 473 |
| pegRNA | 474 |
| pegRNA | 475 |
| pegRNA | 476 |
| pegRNA | 477 |
| pegRNA | 478 |
| pegRNA | 479 |
| pegRNA | 480 |
| pegRNA | 481 |
| pegRNA | 482 |
| ngRNA | 162 |
| ngRNA | 154 |
| ngRNA | 291 |
| ngRNA | 153 |
| ngRNA | 161 |
| ngRNA | 164 |
| ngRNA | 157 |
| ngRNA | 155 |
| ngRNA | 159 |
| ngRNA | 160 |
| ngRNA | 290 |
| ngRNA | 156 |
| ngRNA | 166 |
| ngRNA | 158 |
| ngRNA | 168 |
| ngRNA | 167 |
| ngRNA | 163 |
| ngRNA | 165 |
| ngRNA | 169 |
| ngRNA | 173 |
| ngRNA | 293 |
| ngRNA | 171 |
| ngRNA | 176 |
| ngRNA | 179 |
| ngRNA | 181 |
| ngRNA | 178 |
| ngRNA | 172 |
| ngRNA | 180 |
| ngRNA | 175 |
| ngRNA | 170 |
| ngRNA | 174 |
| ngRNA | 292 |
| ngRNA | 177 |

TABLE 9

| Sequence region | SEQ ID |
|---|---|
| pegRNA Spacer | 483 |
| PBS | 484 |
| PBS | 485 |
| PBS | 486 |
| PBS | 487 |
| PBS | 488 |
| PBS | 489 |
| PBS | 490 |
| PBS | 491 |
| PBS | 492 |
| PBS | 493 |
| PBS | 494 |
| RTT | 495 |
| RTT | 496 |
| RTT | 497 |
| RTT | 498 |
| RTT | 499 |
| RTT | 500 |
| RTT | 501 |
| RTT | 502 |
| RTT | 503 |
| RTT | 504 |
| RTT | 505 |
| RTT | 506 |
| RTT | 507 |
| RTT | 508 |
| RTT | 509 |
| RTT | 510 |
| RTT | 511 |
| RTT | 512 |
| RTT | 513 |
| RTT | 514 |
| RTT | 515 |
| RTT | 516 |
| RTT | 517 |
| RTT | 518 |
| RTT | 519 |
| RTT | 520 |
| RTT | 521 |
| RTT | 522 |
| RTT | 523 |
| RTT | 524 |
| RTT | 525 |
| RTT | 526 |
| RTT | 527 |
| RTT | 528 |
| ngRNA Spacer | 68 |
| ngRNA Spacer | 66 |
| ngRNA Spacer | 61 |
| ngRNA Spacer | 71 |
| ngRNA Spacer | 67 |
| ngRNA Spacer | 46 |
| ngRNA Spacer | 38 |
| ngRNA Spacer | 58 |
| ngRNA Spacer | 36 |
| ngRNA Spacer | 30 |
| ngRNA Spacer | 37 |
| ngRNA Spacer | 29 |
| ngRNA Spacer | 203 |
| ngRNA Spacer | 54 |
| ngRNA Spacer | 59 |
| ngRNA Spacer | 24 |
| ngRNA Spacer | 31 |
| ngRNA Spacer | 53 |
| ngRNA Spacer | 23 |
| ngRNA Spacer | 72 |
| ngRNA Spacer | 44 |
| ngRNA Spacer | 20 |
| ngRNA Spacer | 26 |
| ngRNA Spacer | 22 |
| ngRNA Spacer | 40 |
| ngRNA Spacer | 21 |
| ngRNA Spacer | 60 |
| ngRNA Spacer | 62 |
| ngRNA Spacer | 63 |
| ngRNA Spacer | 19 |
| ngRNA Spacer | 65 |
| ngRNA Spacer | 42 |
| ngRNA Spacer | 64 |
| ngRNA Spacer | 28 |
| ngRNA Spacer | 25 |
| ngRNA Spacer | 18 |
| ngRNA Spacer | 32 |
| ngRNA Spacer | 34 |
| ngRNA Spacer | 50 |
| ngRNA Spacer | 33 |
| ngRNA Spacer | 48 |
| ngRNA Spacer | 39 |
| ngRNA Spacer | 27 |
| ngRNA Spacer | 41 |
| ngRNA Spacer | 529 |
| ngRNA Spacer | 199 |
| ngRNA Spacer | 43 |
| ngRNA Spacer | 70 |
| ngRNA Spacer | 45 |
| ngRNA Spacer | 35 |
| ngRNA Spacer | 200 |
| ngRNA Spacer | 52 |
| ngRNA Spacer | 56 |
| ngRNA Spacer | 55 |
| ngRNA Spacer | 51 |
| ngRNA Spacer | 57 |
| ngRNA Spacer | 49 |
| ngRNA Spacer | 47 |
| ngRNA Spacer | 69 |
| pegRNA | 530 |
| pegRNA | 531 |
| pegRNA | 532 |
| pegRNA | 533 |
| pegRNA | 534 |
| pegRNA | 535 |
| pegRNA | 536 |
| pegRNA | 537 |
| pegRNA | 538 |
| pegRNA | 539 |
| pegRNA | 540 |
| pegRNA | 541 |
| pegRNA | 542 |
| pegRNA | 543 |
| pegRNA | 544 |
| pegRNA | 545 |
| pegRNA | 546 |
| pegRNA | 547 |
| pegRNA | 545 |
| pegRNA | 548 |
| pegRNA | 549 |
| pegRNA | 550 |
| pegRNA | 551 |
| pegRNA | 552 |
| pegRNA | 553 |
| pegRNA | 554 |
| pegRNA | 555 |
| pegRNA | 556 |
| pegRNA | 557 |
| pegRNA | 558 |
| pegRNA | 553 |
| pegRNA | 559 |
| pegRNA | 560 |
| pegRNA | 561 |
| pegRNA | 562 |
| pegRNA | 563 |
| pegRNA | 564 |
| pegRNA | 564 |
| pegRNA | 565 |
| pegRNA | 566 |
| pegRNA | 567 |
| pegRNA | 568 |
| pegRNA | 569 |
| pegRNA | 570 |
| pegRNA | 571 |
| pegRNA | 572 |
| pegRNA | 573 |
| pegRNA | 574 |
| pegRNA | 575 |
| pegRNA | 576 |
| pegRNA | 577 |
| pegRNA | 578 |
| pegRNA | 579 |
| pegRNA | 580 |
| pegRNA | 581 |
| pegRNA | 582 |
| pegRNA | 578 |
| pegRNA | 583 |
| pegRNA | 584 |
| pegRNA | 585 |
| pegRNA | 586 |
| pegRNA | 587 |
| pegRNA | 588 |
| pegRNA | 589 |
| pegRNA | 590 |
| pegRNA | 591 |
| pegRNA | 592 |
| pegRNA | 593 |
| pegRNA | 594 |
| pegRNA | 595 |
| pegRNA | 596 |
| pegRNA | 597 |
| pegRNA | 598 |
| pegRNA | 599 |
| pegRNA | 600 |
| pegRNA | 601 |
| pegRNA | 602 |
| pegRNA | 603 |
| pegRNA | 604 |
| pegRNA | 605 |
| pegRNA | 606 |
| pegRNA | 607 |
| pegRNA | 608 |
| pegRNA | 609 |
| pegRNA | 610 |
| pegRNA | 611 |
| pegRNA | 612 |
| pegRNA | 613 |
| pegRNA | 614 |
| pegRNA | 615 |
| pegRNA | 616 |
| pegRNA | 617 |
| pegRNA | 618 |
| pegRNA | 619 |
| pegRNA | 620 |
| pegRNA | 621 |
| pegRNA | 622 |
| pegRNA | 623 |
| pegRNA | 624 |
| pegRNA | 625 |
| pegRNA | 626 |
| pegRNA | 627 |
| pegRNA | 628 |
| pegRNA | 629 |
| pegRNA | 630 |
| pegRNA | 631 |
| pegRNA | 632 |
| pegRNA | 633 |
| pegRNA | 634 |
| pegRNA | 635 |
| pegRNA | 636 |
| pegRNA | 637 |
| pegRNA | 638 |
| pegRNA | 639 |
| pegRNA | 640 |
| pegRNA | 641 |
| pegRNA | 642 |
| pegRNA | 643 |
| pegRNA | 644 |
| pegRNA | 645 |
| pegRNA | 646 |
| pegRNA | 647 |
| pegRNA | 648 |
| pegRNA | 649 |
| pegRNA | 650 |
| pegRNA | 651 |
| pegRNA | 652 |
| pegRNA | 653 |
| pegRNA | 654 |
| pegRNA | 655 |
| pegRNA | 656 |
| pegRNA | 657 |
| pegRNA | 658 |
| pegRNA | 659 |
| pegRNA | 660 |
| pegRNA | 661 |
| pegRNA | 662 |
| pegRNA | 663 |
| pegRNA | 664 |
| pegRNA | 665 |
| pegRNA | 666 |
| pegRNA | 667 |
| pegRNA | 668 |
| pegRNA | 669 |
| pegRNA | 670 |
| pegRNA | 671 |
| pegRNA | 672 |
| pegRNA | 673 |
| pegRNA | 674 |
| pegRNA | 675 |
| pegRNA | 676 |
| pegRNA | 677 |
| pegRNA | 678 |
| pegRNA | 679 |
| pegRNA | 680 |
| ngRNA | 161 |
| ngRNA | 154 |
| ngRNA | 166 |
| ngRNA | 155 |
| ngRNA | 162 |
| ngRNA | 159 |
| ngRNA | 168 |
| ngRNA | 157 |
| ngRNA | 160 |
| ngRNA | 291 |
| ngRNA | 169 |
| ngRNA | 153 |
| ngRNA | 290 |
| ngRNA | 167 |
| ngRNA | 165 |
| ngRNA | 158 |
| ngRNA | 681 |
| ngRNA | 156 |
| ngRNA | 163 |
| ngRNA | 164 |
| ngRNA | 293 |
| ngRNA | 180 |
| ngRNA | 173 |
| ngRNA | 176 |
| ngRNA | 181 |
| ngRNA | 178 |
| ngRNA | 292 |
| ngRNA | 175 |
| ngRNA | 177 |
| ngRNA | 172 |
| ngRNA | 171 |
| ngRNA | 170 |
| ngRNA | 174 |
| ngRNA | 179 |

TABLE 10

| Sequence region | SEQ ID |
|---|---|
| pegRNA Spacer | 682 |
| PBS | 683 |
| PBS | 684 |
| PBS | 685 |
| PBS | 686 |
| PBS | 687 |
| PBS | 688 |
| PBS | 689 |
| PBS | 690 |
| PBS | 691 |
| PBS | 692 |
| PBS | 693 |
| RTT | 694 |
| RTT | 695 |
| RTT | 696 |
| RTT | 697 |
| RTT | 698 |
| RTT | 699 |
| RTT | 700 |
| RTT | 701 |
| RTT | 702 |
| RTT | 703 |
| RTT | 704 |
| RTT | 705 |
| RTT | 706 |
| RTT | 707 |
| RTT | 708 |
| RTT | 709 |
| RTT | 710 |
| RTT | 711 |
| RTT | 712 |
| RTT | 713 |
| RTT | 714 |
| RTT | 715 |
| RTT | 716 |
| RTT | 717 |
| RTT | 718 |
| RTT | 719 |
| RTT | 720 |
| RTT | 721 |
| RTT | 722 |
| RTT | 723 |
| RTT | 724 |
| RTT | 725 |
| RTT | 726 |
| RTT | 727 |
| RTT | 728 |
| RTT | 729 |
| RTT | 730 |
| RTT | 731 |
| RTT | 732 |
| RTT | 733 |
| RTT | 734 |
| RTT | 735 |
| ngRNA Spacer | 67 |
| ngRNA Spacer | 72 |
| ngRNA Spacer | 34 |
| ngRNA Spacer | 50 |
| ngRNA Spacer | 27 |
| ngRNA Spacer | 36 |
| ngRNA Spacer | 31 |
| ngRNA Spacer | 53 |
| ngRNA Spacer | 57 |
| ngRNA Spacer | 22 |
| ngRNA Spacer | 49 |
| ngRNA Spacer | 43 |
| ngRNA Spacer | 54 |
| ngRNA Spacer | 59 |
| ngRNA Spacer | 56 |
| ngRNA Spacer | 70 |
| ngRNA Spacer | 55 |
| ngRNA Spacer | 44 |
| ngRNA Spacer | 30 |
| ngRNA Spacer | 200 |
| ngRNA Spacer | 203 |
| ngRNA Spacer | 52 |
| ngRNA Spacer | 41 |
| ngRNA Spacer | 51 |
| ngRNA Spacer | 736 |
| ngRNA Spacer | 737 |
| ngRNA Spacer | 32 |
| ngRNA Spacer | 738 |
| ngRNA Spacer | 45 |
| ngRNA Spacer | 60 |
| ngRNA Spacer | 199 |
| ngRNA Spacer | 37 |
| ngRNA Spacer | 18 |
| ngRNA Spacer | 23 |
| ngRNA Spacer | 42 |
| ngRNA Spacer | 21 |
| ngRNA Spacer | 38 |
| ngRNA Spacer | 58 |
| ngRNA Spacer | 33 |
| ngRNA Spacer | 68 |
| ngRNA Spacer | 47 |
| ngRNA Spacer | 40 |
| ngRNA Spacer | 529 |
| ngRNA Spacer | 739 |
| ngRNA Spacer | 46 |
| ngRNA Spacer | 19 |
| ngRNA Spacer | 20 |
| ngRNA Spacer | 26 |
| ngRNA Spacer | 48 |
| ngRNA Spacer | 24 |
| ngRNA Spacer | 35 |
| ngRNA Spacer | 28 |
| ngRNA Spacer | 61 |
| ngRNA Spacer | 66 |
| ngRNA Spacer | 71 |
| ngRNA Spacer | 63 |
| ngRNA Spacer | 64 |
| ngRNA Spacer | 62 |
| ngRNA Spacer | 69 |
| ngRNA Spacer | 740 |
| ngRNA Spacer | 39 |
| ngRNA Spacer | 29 |
| ngRNA Spacer | 25 |
| ngRNA Spacer | 65 |
| pegRNA | 741 |
| pegRNA | 742 |
| pegRNA | 743 |
| pegRNA | 744 |
| pegRNA | 745 |
| pegRNA | 746 |
| pegRNA | 747 |
| pegRNA | 748 |
| pegRNA | 749 |
| pegRNA | 750 |
| pegRNA | 751 |
| pegRNA | 752 |
| pegRNA | 753 |
| pegRNA | 754 |
| pegRNA | 755 |
| pegRNA | 756 |
| pegRNA | 757 |
| pegRNA | 758 |
| pegRNA | 760 |
| pegRNA | 761 |
| pegRNA | 762 |
| pegRNA | 763 |
| pegRNA | 764 |
| pegRNA | 765 |
| pegRNA | 766 |
| pegRNA | 767 |
| pegRNA | 768 |
| pegRNA | 769 |
| pegRNA | 770 |
| pegRNA | 771 |
| pegRNA | 772 |
| pegRNA | 773 |
| pegRNA | 774 |
| pegRNA | 775 |
| pegRNA | 776 |
| pegRNA | 777 |
| pegRNA | 778 |
| pegRNA | 779 |
| pegRNA | 780 |
| pegRNA | 781 |
| pegRNA | 782 |
| pegRNA | 783 |
| pegRNA | 784 |
| pegRNA | 785 |
| pegRNA | 786 |
| pegRNA | 787 |
| pegRNA | 788 |
| pegRNA | 789 |
| pegRNA | 790 |
| pegRNA | 791 |
| pegRNA | 792 |
| pegRNA | 793 |
| pegRNA | 794 |
| pegRNA | 795 |
| pegRNA | 796 |
| pegRNA | 797 |
| pegRNA | 798 |
| pegRNA | 799 |
| pegRNA | 800 |
| pegRNA | 801 |
| pegRNA | 802 |
| pegRNA | 803 |
| pegRNA | 804 |
| pegRNA | 806 |
| pegRNA | 807 |
| pegRNA | 808 |
| pegRNA | 809 |
| pegRNA | 810 |
| pegRNA | 811 |
| pegRNA | 812 |
| pegRNA | 813 |
| pegRNA | 814 |
| pegRNA | 815 |
| pegRNA | 816 |
| pegRNA | 817 |
| pegRNA | 818 |
| pegRNA | 819 |
| pegRNA | 820 |
| pegRNA | 821 |
| pegRNA | 822 |
| pegRNA | 823 |
| pegRNA | 824 |
| pegRNA | 825 |
| pegRNA | 826 |
| pegRNA | 827 |
| pegRNA | 828 |
| pegRNA | 829 |
| pegRNA | 830 |
| pegRNA | 831 |
| pegRNA | 832 |
| pegRNA | 833 |
| pegRNA | 834 |
| pegRNA | 835 |
| pegRNA | 836 |
| pegRNA | 837 |
| pegRNA | 838 |
| pegRNA | 839 |
| pegRNA | 840 |
| pegRNA | 841 |
| pegRNA | 842 |
| pegRNA | 843 |
| pegRNA | 844 |
| pegRNA | 845 |
| pegRNA | 846 |
| pegRNA | 847 |
| pegRNA | 848 |
| pegRNA | 849 |
| pegRNA | 850 |
| pegRNA | 851 |
| pegRNA | 852 |
| pegRNA | 853 |
| pegRNA | 854 |
| pegRNA | 855 |
| pegRNA | 856 |
| pegRNA | 857 |
| pegRNA | 858 |
| pegRNA | 859 |
| pegRNA | 860 |
| pegRNA | 861 |
| pegRNA | 862 |
| pegRNA | 863 |
| pegRNA | 864 |
| pegRNA | 865 |
| pegRNA | 866 |
| pegRNA | 867 |
| pegRNA | 868 |
| pegRNA | 869 |
| pegRNA | 870 |
| pegRNA | 871 |
| pegRNA | 872 |
| pegRNA | 873 |
| pegRNA | 874 |
| pegRNA | 875 |
| pegRNA | 876 |
| pegRNA | 877 |
| pegRNA | 878 |
| pegRNA | 879 |
| pegRNA | 880 |
| pegRNA | 881 |
| pegRNA | 882 |
| pegRNA | 883 |
| pegRNA | 884 |
| pegRNA | 885 |
| pegRNA | 886 |
| pegRNA | 887 |
| pegRNA | 888 |
| pegRNA | 889 |
| pegRNA | 890 |
| pegRNA | 891 |
| pegRNA | 892 |
| pegRNA | 893 |
| pegRNA | 894 |
| pegRNA | 895 |
| pegRNA | 896 |
| pegRNA | 897 |
| pegRNA | 898 |
| pegRNA | 899 |
| pegRNA | 900 |
| pegRNA | 901 |
| pegRNA | 902 |
| pegRNA | 903 |
| pegRNA | 904 |
| pegRNA | 905 |
| pegRNA | 906 |
| pegRNA | 907 |
| pegRNA | 908 |
| pegRNA | 909 |
| pegRNA | 910 |
| pegRNA | 911 |
| pegRNA | 912 |
| pegRNA | 913 |
| pegRNA | 914 |
| pegRNA | 915 |
| pegRNA | 916 |
| pegRNA | 917 |
| pegRNA | 918 |
| pegRNA | 919 |
| pegRNA | 920 |
| pegRNA | 921 |
| pegRNA | 922 |
| pegRNA | 923 |
| pegRNA | 924 |
| pegRNA | 925 |
| pegRNA | 926 |
| pegRNA | 927 |
| pegRNA | 928 |
| pegRNA | 929 |
| pegRNA | 930 |
| pegRNA | 931 |
| pegRNA | 932 |
| pegRNA | 933 |
| pegRNA | 934 |
| pegRNA | 935 |
| pegRNA | 936 |
| pegRNA | 937 |
| pegRNA | 938 |
| pegRNA | 939 |
| pegRNA | 940 |
| pegRNA | 941 |
| pegRNA | 942 |
| pegRNA | 943 |
| pegRNA | 944 |
| pegRNA | 945 |
| pegRNA | 946 |
| pegRNA | 947 |
| pegRNA | 948 |
| pegRNA | 949 |
| pegRNA | 950 |
| pegRNA | 951 |
| pegRNA | 952 |
| pegRNA | 953 |
| pegRNA | 954 |
| pegRNA | 955 |
| pegRNA | 956 |
| pegRNA | 957 |
| pegRNA | 958 |
| pegRNA | 959 |
| pegRNA | 960 |
| pegRNA | 961 |
| pegRNA | 962 |
| pegRNA | 963 |
| pegRNA | 964 |
| pegRNA | 965 |
| pegRNA | 966 |
| pegRNA | 967 |
| pegRNA | 968 |
| pegRNA | 969 |
| pegRNA | 970 |
| pegRNA | 971 |
| pegRNA | 972 |
| pegRNA | 973 |
| pegRNA | 974 |
| pegRNA | 975 |
| pegRNA | 976 |
| pegRNA | 977 |
| pegRNA | 978 |
| pegRNA | 979 |
| pegRNA | 980 |
| pegRNA | 981 |
| pegRNA | 982 |
| pegRNA | 983 |
| pegRNA | 984 |
| pegRNA | 985 |
| pegRNA | 986 |
| pegRNA | 987 |
| pegRNA | 988 |
| pegRNA | 989 |
| pegRNA | 990 |
| pegRNA | 991 |
| pegRNA | 992 |
| pegRNA | 993 |
| pegRNA | 994 |
| pegRNA | 995 |
| pegRNA | 996 |
| pegRNA | 997 |
| pegRNA | 998 |
| pegRNA | 999 |
| pegRNA | 1000 |
| pegRNA | 1001 |
| pegRNA | 1002 |
| pegRNA | 1003 |
| pegRNA | 1004 |
| pegRNA | 1005 |
| pegRNA | 1006 |
| pegRNA | 1007 |
| pegRNA | 1008 |
| pegRNA | 1009 |
| pegRNA | 1010 |
| pegRNA | 1011 |
| pegRNA | 1012 |
| pegRNA | 1013 |
| pegRNA | 1014 |
| pegRNA | 1015 |
| pegRNA | 1016 |
| pegRNA | 1017 |
| pegRNA | 1018 |
| pegRNA | 1019 |
| pegRNA | 1020 |
| pegRNA | 1021 |
| pegRNA | 1022 |
| pegRNA | 1023 |
| pegRNA | 1024 |
| pegRNA | 1025 |
| pegRNA | 1026 |
| pegRNA | 1027 |
| pegRNA | 1028 |
| pegRNA | 1029 |
| pegRNA | 1030 |
| pegRNA | 1031 |
| pegRNA | 1032 |
| pegRNA | 1033 |
| pegRNA | 1034 |
| pegRNA | 1035 |
| pegRNA | 1036 |
| pegRNA | 1037 |
| pegRNA | 1038 |
| pegRNA | 1039 |
| pegRNA | 1040 |
| pegRNA | 1041 |
| pegRNA | 1042 |
| pegRNA | 1043 |
| pegRNA | 1044 |
| pegRNA | 1045 |
| pegRNA | 1046 |
| pegRNA | 1047 |
| pegRNA | 1048 |
| pegRNA | 1049 |
| pegRNA | 1050 |
| pegRNA | 1051 |
| pegRNA | 1052 |
| pegRNA | 1053 |
| pegRNA | 1054 |
| pegRNA | 1055 |
| pegRNA | 1056 |
| pegRNA | 1057 |
| pegRNA | 1058 |
| pegRNA | 1059 |
| pegRNA | 1060 |
| pegRNA | 1061 |
| pegRNA | 1062 |
| pegRNA | 1063 |
| pegRNA | 1064 |
| pegRNA | 1065 |
| pegRNA | 1066 |
| pegRNA | 1067 |
| pegRNA | 1068 |
| pegRNA | 1069 |
| pegRNA | 1070 |
| pegRNA | 1071 |
| pegRNA | 1072 |
| pegRNA | 1073 |
| pegRNA | 1074 |
| pegRNA | 1075 |
| pegRNA | 1076 |
| pegRNA | 1077 |
| pegRNA | 1078 |
| pegRNA | 1079 |
| pegRNA | 1080 |
| pegRNA | 1081 |
| pegRNA | 1082 |
| pegRNA | 1083 |
| pegRNA | 1084 |
| pegRNA | 1085 |
| pegRNA | 1086 |
| pegRNA | 1087 |
| pegRNA | 1088 |
| pegRNA | 1089 |
| pegRNA | 1090 |
| pegRNA | 1091 |
| pegRNA | 1092 |
| pegRNA | 1093 |
| pegRNA | 1094 |
| pegRNA | 1095 |
| pegRNA | 1096 |
| pegRNA | 1097 |
| pegRNA | 1098 |
| pegRNA | 1099 |
| pegRNA | 1100 |
| pegRNA | 1101 |
| pegRNA | 1102 |
| pegRNA | 1103 |
| pegRNA | 1104 |
| pegRNA | 1105 |
| pegRNA | 1106 |
| pegRNA | 1107 |
| pegRNA | 1108 |
| pegRNA | 1109 |
| pegRNA | 1110 |
| pegRNA | 1111 |
| pegRNA | 1112 |
| pegRNA | 1113 |
| pegRNA | 1114 |
| pegRNA | 1115 |
| pegRNA | 1116 |
| pegRNA | 1117 |
| pegRNA | 1118 |
| pegRNA | 1119 |
| pegRNA | 1120 |
| pegRNA | 1121 |
| pegRNA | 1122 |
| pegRNA | 1123 |
| pegRNA | 1124 |
| pegRNA | 1125 |
| pegRNA | 1126 |
| pegRNA | 1127 |
| pegRNA | 1128 |
| pegRNA | 1129 |
| pegRNA | 1130 |
| pegRNA | 1131 |
| pegRNA | 1132 |
| pegRNA | 1133 |
| pegRNA | 1134 |
| pegRNA | 1135 |
| pegRNA | 1136 |
| pegRNA | 1137 |
| pegRNA | 1138 |
| pegRNA | 1139 |
| pegRNA | 1140 |
| pegRNA | 1141 |
| pegRNA | 1142 |
| pegRNA | 1143 |
| pegRNA | 1144 |
| pegRNA | 1145 |
| pegRNA | 1146 |
| pegRNA | 1147 |
| pegRNA | 1148 |
| pegRNA | 1149 |
| pegRNA | 1150 |
| pegRNA | 1151 |
| pegRNA | 1152 |
| pegRNA | 1153 |
| pegRNA | 1154 |
| pegRNA | 1155 |
| pegRNA | 1156 |
| pegRNA | 1157 |
| pegRNA | 1158 |
| pegRNA | 1159 |
| pegRNA | 1160 |
| pegRNA | 1161 |
| pegRNA | 1162 |
| pegRNA | 1163 |
| pegRNA | 1164 |
| pegRNA | 1165 |
| pegRNA | 1166 |
| pegRNA | 1167 |
| pegRNA | 1168 |
| pegRNA | 1169 |
| pegRNA | 1170 |
| pegRNA | 1171 |
| pegRNA | 1172 |
| pegRNA | 1173 |
| pegRNA | 1174 |
| pegRNA | 1175 |
| pegRNA | 1176 |
| pegRNA | 1177 |
| pegRNA | 1178 |
| pegRNA | 1179 |
| pegRNA | 1180 |
| pegRNA | 1181 |
| pegRNA | 1182 |
| pegRNA | 1183 |
| pegRNA | 1184 |
| pegRNA | 1185 |
| pegRNA | 1186 |
| pegRNA | 1187 |
| pegRNA | 1188 |
| pegRNA | 1189 |
| pegRNA | 1190 |
| pegRNA | 1191 |
| pegRNA | 1192 |
| pegRNA | 1193 |
| pegRNA | 1194 |
| pegRNA | 1195 |
| pegRNA | 1196 |
| pegRNA | 1197 |
| pegRNA | 1198 |
| pegRNA | 1199 |
| pegRNA | 1200 |
| pegRNA | 1201 |
| pegRNA | 1202 |
| pegRNA | 1203 |
| pegRNA | 1204 |
| pegRNA | 1205 |
| pegRNA | 1206 |
| pegRNA | 1207 |
| pegRNA | 1208 |
| pegRNA | 1209 |
| pegRNA | 1210 |
| pegRNA | 1211 |
| pegRNA | 1212 |
| pegRNA | 1213 |
| pegRNA | 1214 |
| pegRNA | 1215 |
| pegRNA | 1216 |
| pegRNA | 1217 |
| pegRNA | 1218 |
| pegRNA | 1219 |
| pegRNA | 1220 |
| pegRNA | 1221 |
| pegRNA | 1222 |
| pegRNA | 1223 |
| pegRNA | 1224 |
| pegRNA | 1225 |
| pegRNA | 1226 |
| pegRNA | 1227 |
| pegRNA | 1228 |
| pegRNA | 1229 |
| pegRNA | 1230 |
| pegRNA | 1231 |
| pegRNA | 1232 |
| pegRNA | 1233 |
| pegRNA | 1234 |
| pegRNA | 1235 |
| pegRNA | 1236 |
| pegRNA | 1237 |
| pegRNA | 1238 |
| pegRNA | 1239 |
| pegRNA | 1240 |
| pegRNA | 1241 |
| pegRNA | 1242 |
| pegRNA | 1243 |
| pegRNA | 1244 |
| pegRNA | 1245 |
| pegRNA | 1246 |
| pegRNA | 1247 |
| pegRNA | 1248 |
| pegRNA | 1249 |
| pegRNA | 1250 |
| pegRNA | 1251 |
| pegRNA | 1252 |
| pegRNA | 1253 |
| pegRNA | 1254 |
| pegRNA | 1255 |
| pegRNA | 1256 |
| pegRNA | 1257 |
| pegRNA | 1258 |
| pegRNA | 1259 |
| pegRNA | 1260 |
| pegRNA | 1261 |
| pegRNA | 1262 |
| pegRNA | 1263 |
| pegRNA | 1264 |
| pegRNA | 1265 |
| pegRNA | 1266 |
| pegRNA | 1267 |
| pegRNA | 1268 |
| pegRNA | 1269 |
| pegRNA | 1270 |
| pegRNA | 1271 |
| pegRNA | 1272 |
| pegRNA | 1273 |
| pegRNA | 1274 |
| pegRNA | 1275 |
| pegRNA | 1276 |
| pegRNA | 1277 |
| pegRNA | 1278 |
| pegRNA | 1279 |
| pegRNA | 1280 |
| pegRNA | 1281 |
| pegRNA | 1282 |
| pegRNA | 1283 |
| pegRNA | 1284 |
| pegRNA | 1285 |
| pegRNA | 1286 |
| pegRNA | 1287 |
| pegRNA | 1288 |
| pegRNA | 1289 |
| pegRNA | 1290 |
| pegRNA | 1291 |
| pegRNA | 1292 |
| pegRNA | 1293 |
| pegRNA | 1294 |
| pegRNA | 1295 |
| pegRNA | 1296 |
| pegRNA | 1297 |
| pegRNA | 1298 |
| pegRNA | 1299 |
| pegRNA | 1300 |
| pegRNA | 1301 |
| pegRNA | 1302 |
| pegRNA | 1303 |
| pegRNA | 1304 |
| pegRNA | 1305 |
| pegRNA | 1306 |
| pegRNA | 1307 |
| pegRNA | 1308 |
| pegRNA | 1309 |
| pegRNA | 1310 |
| pegRNA | 1311 |
| pegRNA | 1312 |
| pegRNA | 1313 |
| pegRNA | 1314 |
| pegRNA | 1315 |
| pegRNA | 1316 |
| pegRNA | 1317 |
| pegRNA | 1318 |
| pegRNA | 1319 |
| pegRNA | 1320 |
| pegRNA | 1321 |
| pegRNA | 1322 |
| pegRNA | 1323 |
| pegRNA | 1324 |
| pegRNA | 1325 |
| pegRNA | 1326 |
| pegRNA | 1327 |
| pegRNA | 1328 |
| pegRNA | 1329 |
| pegRNA | 1330 |
| pegRNA | 1331 |
| pegRNA | 1332 |
| pegRNA | 1333 |
| pegRNA | 1334 |
| pegRNA | 1335 |
| pegRNA | 1336 |
| pegRNA | 1337 |
| pegRNA | 1338 |
| pegRNA | 1339 |
| pegRNA | 1340 |
| pegRNA | 1341 |
| pegRNA | 1342 |
| pegRNA | 1343 |
| pegRNA | 1344 |
| pegRNA | 1345 |
| pegRNA | 1346 |
| pegRNA | 1347 |
| pegRNA | 1348 |
| pegRNA | 1349 |
| pegRNA | 1350 |
| pegRNA | 1351 |
| pegRNA | 1352 |
| pegRNA | 1353 |
| pegRNA | 1354 |
| pegRNA | 1355 |
| pegRNA | 1356 |
| pegRNA | 1357 |
| pegRNA | 1358 |
| pegRNA | 1359 |
| pegRNA | 1360 |
| pegRNA | 1361 |
| pegRNA | 1362 |
| pegRNA | 1363 |
| pegRNA | 1364 |
| pegRNA | 1365 |
| pegRNA | 1366 |
| pegRNA | 1367 |
| pegRNA | 1368 |
| pegRNA | 1369 |
| pegRNA | 1370 |
| pegRNA | 1371 |
| pegRNA | 1372 |
| pegRNA | 1373 |
| pegRNA | 1374 |
| pegRNA | 1375 |
| pegRNA | 1376 |
| pegRNA | 1377 |
| pegRNA | 1378 |
| pegRNA | 1379 |
| pegRNA | 1380 |
| pegRNA | 1381 |
| pegRNA | 1382 |
| pegRNA | 1383 |
| pegRNA | 1384 |
| pegRNA | 1385 |
| pegRNA | 1386 |
| pegRNA | 1387 |
| pegRNA | 1388 |
| pegRNA | 1389 |
| pegRNA | 1390 |
| pegRNA | 1391 |
| pegRNA | 1392 |
| pegRNA | 1393 |
| pegRNA | 1394 |
| pegRNA | 1395 |
| pegRNA | 1396 |
| pegRNA | 1397 |
| pegRNA | 1398 |
| pegRNA | 1399 |
| pegRNA | 1400 |
| pegRNA | 1401 |
| pegRNA | 1402 |
| pegRNA | 1403 |
| pegRNA | 1404 |
| pegRNA | 1405 |
| pegRNA | 1406 |
| pegRNA | 1407 |
| pegRNA | 1408 |
| pegRNA | 1409 |
| pegRNA | 1410 |
| pegRNA | 1411 |
| pegRNA | 1412 |
| pegRNA | 1413 |
| pegRNA | 1414 |
| pegRNA | 1415 |
| pegRNA | 1416 |
| pegRNA | 1417 |
| pegRNA | 1418 |
| pegRNA | 1419 |
| pegRNA | 1420 |
| pegRNA | 1421 |
| pegRNA | 1422 |
| pegRNA | 1423 |
| pegRNA | 1424 |
| pegRNA | 1425 |
| pegRNA | 1426 |
| pegRNA | 1427 |
| pegRNA | 1428 |
| pegRNA | 1429 |
| pegRNA | 1430 |
| pegRNA | 1431 |
| pegRNA | 1432 |
| pegRNA | 1433 |
| pegRNA | 1434 |
| pegRNA | 1435 |
| pegRNA | 1436 |
| pegRNA | 1437 |
| pegRNA | 1438 |
| pegRNA | 1439 |
| pegRNA | 1440 |
| pegRNA | 1441 |
| pegRNA | 1442 |
| pegRNA | 1443 |
| pegRNA | 1444 |
| pegRNA | 1445 |
| pegRNA | 1446 |
| pegRNA | 1447 |
| pegRNA | 1448 |
| pegRNA | 1450 |
| pegRNA | 1451 |
| pegRNA | 1452 |
| pegRNA | 1453 |
| pegRNA | 1454 |
| pegRNA | 1455 |
| pegRNA | 1456 |
| pegRNA | 1457 |
| pegRNA | 1458 |
| pegRNA | 1459 |
| pegRNA | 1460 |
| pegRNA | 1461 |
| pegRNA | 1462 |
| pegRNA | 1463 |
| pegRNA | 1464 |
| pegRNA | 1465 |
| pegRNA | 1466 |
| pegRNA | 1467 |
| pegRNA | 1468 |
| pegRNA | 1469 |
| pegRNA | 1470 |
| pegRNA | 1471 |
| pegRNA | 1472 |
| pegRNA | 1473 |
| pegRNA | 1474 |
| pegRNA | 1475 |
| pegRNA | 1476 |
| pegRNA | 1477 |
| pegRNA | 1478 |
| pegRNA | 1479 |
| pegRNA | 1480 |
| pegRNA | 1481 |
| pegRNA | 1482 |
| pegRNA | 1483 |
| pegRNA | 1484 |
| pegRNA | 1485 |
| pegRNA | 1486 |
| pegRNA | 1487 |
| pegRNA | 1488 |
| pegRNA | 1489 |
| pegRNA | 1490 |
| pegRNA | 1491 |
| pegRNA | 1492 |
| pegRNA | 1493 |
| pegRNA | 1494 |
| pegRNA | 1495 |
| pegRNA | 1496 |
| pegRNA | 1497 |
| pegRNA | 1498 |
| pegRNA | 1499 |
| pegRNA | 1500 |
| ngRNA | 291 |
| ngRNA | 164 |
| ngRNA | 681 |
| ngRNA | 1501 |
| ngRNA | 166 |
| ngRNA | 158 |
| ngRNA | 161 |
| ngRNA | 157 |
| ngRNA | 154 |
| ngRNA | 153 |
| ngRNA | 167 |
| ngRNA | 168 |
| ngRNA | 169 |
| ngRNA | 155 |
| ngRNA | 1502 |
| ngRNA | 160 |
| ngRNA | 1503 |
| ngRNA | 156 |
| ngRNA | 159 |
| ngRNA | 162 |
| ngRNA | 165 |
| ngRNA | 163 |
| ngRNA | 290 |
| ngRNA | 1504 |
| ngRNA | 177 |
| ngRNA | 178 |
| ngRNA | 171 |
| ngRNA | 175 |
| ngRNA | 173 |
| ngRNA | 180 |
| ngRNA | 293 |
| ngRNA | 176 |
| ngRNA | 181 |
| ngRNA | 292 |
| ngRNA | 174 |
| ngRNA | 170 |
| ngRNA | 172 |
| ngRNA | 179 |

TABLE 11

| Sequence region | SEQ ID |
|---|---|
| pegRNA Spacer | 1505 |
| PBS | 1506 |
| PBS | 1507 |
| PBS | 1508 |
| PBS | 1509 |
| PBS | 1510 |
| PBS | 1511 |
| PBS | 1512 |
| PBS | 1513 |
| PBS | 1514 |
| PBS | 1515 |
| PBS | 1516 |
| RTT | 1517 |
| RTT | 1518 |
| RTT | 1519 |
| RTT | 1520 |
| RTT | 1521 |
| RTT | 1522 |
| RTT | 1523 |
| RTT | 1524 |
| RTT | 1525 |
| RTT | 1526 |
| RTT | 1527 |
| RTT | 1528 |
| RTT | 1529 |
| RTT | 1530 |
| RTT | 1531 |
| RTT | 1532 |
| RTT | 1533 |
| RTT | 1534 |
| RTT | 1535 |
| RTT | 1536 |
| RTT | 1537 |
| RTT | 1538 |
| RTT | 1539 |
| RTT | 1540 |
| RTT | 1541 |
| RTT | 1542 |
| RTT | 1543 |
| RTT | 1544 |
| RTT | 1545 |
| RTT | 1546 |
| ngRNA Spacer | 65 |
| ngRNA Spacer | 63 |
| ngRNA Spacer | 62 |
| ngRNA Spacer | 66 |
| ngRNA Spacer | 61 |
| ngRNA Spacer | 71 |
| ngRNA Spacer | 53 |
| ngRNA Spacer | 37 |
| ngRNA Spacer | 44 |
| ngRNA Spacer | 529 |
| ngRNA Spacer | 42 |
| ngRNA Spacer | 58 |
| ngRNA Spacer | 50 |
| ngRNA Spacer | 31 |
| ngRNA Spacer | 19 |
| ngRNA Spacer | 737 |
| ngRNA Spacer | 738 |
| ngRNA Spacer | 36 |
| ngRNA Spacer | 739 |
| ngRNA Spacer | 34 |
| ngRNA Spacer | 203 |
| ngRNA Spacer | 49 |
| ngRNA Spacer | 54 |
| ngRNA Spacer | 52 |
| ngRNA Spacer | 18 |
| ngRNA Spacer | 43 |
| ngRNA Spacer | 57 |
| ngRNA Spacer | 56 |
| ngRNA Spacer | 64 |
| ngRNA Spacer | 24 |
| ngRNA Spacer | 23 |
| ngRNA Spacer | 72 |
| ngRNA Spacer | 51 |
| ngRNA Spacer | 70 |
| ngRNA Spacer | 48 |
| ngRNA Spacer | 60 |
| ngRNA Spacer | 21 |
| ngRNA Spacer | 20 |
| ngRNA Spacer | 25 |
| ngRNA Spacer | 40 |
| ngRNA Spacer | 22 |
| ngRNA Spacer | 38 |
| ngRNA Spacer | 59 |
| ngRNA Spacer | 39 |
| ngRNA Spacer | 46 |
| ngRNA Spacer | 26 |
| ngRNA Spacer | 29 |
| ngRNA Spacer | 67 |
| ngRNA Spacer | 28 |
| ngRNA Spacer | 69 |
| ngRNA Spacer | 30 |
| ngRNA Spacer | 41 |
| ngRNA Spacer | 740 |
| ngRNA Spacer | 68 |
| ngRNA Spacer | 32 |
| ngRNA Spacer | 35 |
| ngRNA Spacer | 736 |
| ngRNA Spacer | 47 |
| ngRNA Spacer | 199 |
| ngRNA Spacer | 33 |
| ngRNA Spacer | 55 |
| ngRNA Spacer | 45 |
| ngRNA Spacer | 27 |
| ngRNA Spacer | 200 |
| pegRNA | 1547 |
| pegRNA | 1548 |
| pegRNA | 1549 |
| pegRNA | 1550 |
| pegRNA | 1551 |
| pegRNA | 1552 |
| pegRNA | 1553 |
| pegRNA | 1554 |
| pegRNA | 1555 |
| pegRNA | 1556 |
| pegRNA | 1557 |
| pegRNA | 1558 |
| pegRNA | 1559 |
| pegRNA | 1560 |
| pegRNA | 1561 |
| pegRNA | 1562 |
| pegRNA | 1563 |
| pegRNA | 1564 |
| pegRNA | 1565 |
| pegRNA | 1566 |
| pegRNA | 1567 |
| pegRNA | 1568 |
| pegRNA | 1569 |
| pegRNA | 1570 |
| pegRNA | 1571 |
| pegRNA | 1572 |
| pegRNA | 1573 |
| pegRNA | 1574 |
| pegRNA | 1575 |
| pegRNA | 1576 |
| pegRNA | 1577 |
| pegRNA | 1578 |
| pegRNA | 1579 |
| pegRNA | 1580 |
| pegRNA | 1581 |
| pegRNA | 1582 |
| pegRNA | 1583 |
| pegRNA | 1584 |
| pegRNA | 1585 |
| pegRNA | 1586 |
| pegRNA | 1587 |
| pegRNA | 1588 |
| pegRNA | 1589 |
| pegRNA | 1590 |
| pegRNA | 1591 |
| pegRNA | 1592 |
| pegRNA | 1593 |
| pegRNA | 1594 |
| pegRNA | 1595 |
| pegRNA | 1596 |
| pegRNA | 1597 |
| pegRNA | 1598 |
| pegRNA | 1599 |
| pegRNA | 1600 |
| pegRNA | 1601 |
| pegRNA | 1602 |
| pegRNA | 1603 |
| pegRNA | 1604 |
| pegRNA | 1605 |
| pegRNA | 1606 |
| pegRNA | 1607 |
| pegRNA | 1608 |
| pegRNA | 1609 |
| pegRNA | 1610 |
| pegRNA | 1611 |
| pegRNA | 1612 |
| pegRNA | 1613 |
| pegRNA | 1614 |
| pegRNA | 1615 |
| pegRNA | 1616 |
| pegRNA | 1617 |
| pegRNA | 1618 |
| pegRNA | 1619 |
| pegRNA | 1620 |
| pegRNA | 1621 |
| pegRNA | 1622 |
| pegRNA | 1623 |
| pegRNA | 1624 |
| pegRNA | 1625 |
| pegRNA | 1626 |
| pegRNA | 1627 |
| pegRNA | 1628 |
| pegRNA | 1629 |
| pegRNA | 1630 |
| pegRNA | 1631 |
| pegRNA | 1632 |
| pegRNA | 1633 |
| pegRNA | 1634 |
| pegRNA | 1635 |
| pegRNA | 1636 |
| pegRNA | 1637 |
| pegRNA | 1638 |
| pegRNA | 1639 |
| pegRNA | 1640 |
| pegRNA | 1641 |
| pegRNA | 1642 |
| pegRNA | 1643 |
| pegRNA | 1644 |
| pegRNA | 1645 |
| pegRNA | 1646 |
| pegRNA | 1647 |
| pegRNA | 1648 |
| pegRNA | 1649 |
| pegRNA | 1650 |
| pegRNA | 1651 |
| pegRNA | 1652 |
| pegRNA | 1653 |
| pegRNA | 1654 |
| pegRNA | 1655 |
| pegRNA | 1656 |
| pegRNA | 1657 |
| pegRNA | 1658 |
| pegRNA | 1659 |
| pegRNA | 1660 |
| pegRNA | 1661 |
| pegRNA | 1662 |
| pegRNA | 1663 |
| pegRNA | 1664 |
| pegRNA | 1665 |
| pegRNA | 1666 |
| pegRNA | 1667 |
| pegRNA | 1668 |
| pegRNA | 1669 |
| pegRNA | 1670 |
| pegRNA | 1671 |
| pegRNA | 1672 |
| pegRNA | 1673 |
| pegRNA | 1674 |
| pegRNA | 1675 |
| pegRNA | 1676 |
| pegRNA | 1677 |
| pegRNA | 1678 |
| pegRNA | 1679 |
| pegRNA | 1680 |
| pegRNA | 1681 |
| pegRNA | 1682 |
| pegRNA | 1683 |
| pegRNA | 1684 |
| pegRNA | 1685 |
| pegRNA | 1686 |
| pegRNA | 1687 |
| pegRNA | 1688 |
| pegRNA | 1689 |
| pegRNA | 1690 |
| pegRNA | 1691 |
| pegRNA | 1692 |
| pegRNA | 1693 |
| pegRNA | 1694 |
| pegRNA | 1695 |
| pegRNA | 1696 |
| pegRNA | 1697 |
| pegRNA | 1698 |
| pegRNA | 1699 |
| pegRNA | 1700 |
| pegRNA | 1701 |
| pegRNA | 1702 |
| pegRNA | 1703 |
| pegRNA | 1704 |
| pegRNA | 1705 |
| pegRNA | 1706 |
| pegRNA | 1707 |
| pegRNA | 1708 |
| pegRNA | 1709 |
| pegRNA | 1710 |
| pegRNA | 1711 |
| pegRNA | 1712 |
| pegRNA | 1713 |
| pegRNA | 1714 |
| pegRNA | 1715 |
| pegRNA | 1716 |
| pegRNA | 1717 |
| pegRNA | 1718 |
| pegRNA | 1719 |
| pegRNA | 1720 |
| pegRNA | 1721 |
| pegRNA | 1722 |
| pegRNA | 1723 |
| pegRNA | 1724 |
| pegRNA | 1725 |
| pegRNA | 1726 |
| pegRNA | 1727 |
| pegRNA | 1728 |
| pegRNA | 1729 |
| pegRNA | 1730 |
| pegRNA | 1731 |
| pegRNA | 1732 |
| pegRNA | 1733 |
| pegRNA | 1734 |
| pegRNA | 1735 |
| pegRNA | 1736 |
| pegRNA | 1737 |
| pegRNA | 1738 |
| pegRNA | 1739 |
| pegRNA | 1740 |
| pegRNA | 1741 |
| pegRNA | 1742 |
| pegRNA | 1743 |
| pegRNA | 1744 |
| pegRNA | 1745 |
| pegRNA | 1746 |
| pegRNA | 1747 |
| pegRNA | 1748 |
| pegRNA | 1749 |
| pegRNA | 1750 |
| pegRNA | 1751 |
| pegRNA | 1752 |
| pegRNA | 1753 |
| pegRNA | 1754 |
| pegRNA | 1755 |
| pegRNA | 1756 |
| pegRNA | 1757 |
| pegRNA | 1758 |
| pegRNA | 1759 |
| pegRNA | 1760 |
| pegRNA | 1761 |
| pegRNA | 1762 |
| pegRNA | 1763 |
| pegRNA | 1764 |
| pegRNA | 1765 |
| pegRNA | 1766 |
| pegRNA | 1767 |
| pegRNA | 1768 |
| pegRNA | 1769 |
| pegRNA | 1770 |
| pegRNA | 1771 |
| pegRNA | 1772 |
| pegRNA | 1773 |
| pegRNA | 1774 |
| pegRNA | 1775 |
| pegRNA | 1776 |
| pegRNA | 1777 |
| pegRNA | 1778 |
| pegRNA | 1779 |
| pegRNA | 1780 |
| pegRNA | 1781 |
| pegRNA | 1782 |
| pegRNA | 1783 |
| pegRNA | 1784 |
| pegRNA | 1785 |
| pegRNA | 1786 |
| pegRNA | 1787 |
| pegRNA | 1788 |
| pegRNA | 1789 |
| pegRNA | 1790 |
| pegRNA | 1791 |
| pegRNA | 1792 |
| pegRNA | 1793 |
| pegRNA | 1794 |
| pegRNA | 1795 |
| pegRNA | 1796 |
| pegRNA | 1797 |
| pegRNA | 1798 |
| pegRNA | 1799 |
| pegRNA | 1800 |
| pegRNA | 1801 |
| pegRNA | 1802 |
| pegRNA | 1803 |
| pegRNA | 1804 |
| pegRNA | 1805 |
| pegRNA | 1806 |
| pegRNA | 1807 |
| pegRNA | 1808 |
| pegRNA | 1809 |
| pegRNA | 1810 |
| pegRNA | 1811 |
| pegRNA | 1812 |
| pegRNA | 1813 |
| pegRNA | 1814 |
| pegRNA | 1815 |
| pegRNA | 1816 |
| pegRNA | 1817 |
| pegRNA | 1818 |
| pegRNA | 1819 |
| pegRNA | 1820 |
| pegRNA | 1821 |
| pegRNA | 1822 |
| pegRNA | 1823 |
| pegRNA | 1824 |
| pegRNA | 1825 |
| pegRNA | 1826 |
| pegRNA | 1827 |
| pegRNA | 1828 |
| pegRNA | 1829 |
| pegRNA | 1830 |
| pegRNA | 1831 |
| pegRNA | 1832 |
| pegRNA | 1833 |
| pegRNA | 1834 |
| pegRNA | 1835 |
| pegRNA | 1836 |
| pegRNA | 1837 |
| pegRNA | 1838 |
| pegRNA | 1839 |
| pegRNA | 1840 |
| pegRNA | 1841 |
| pegRNA | 1842 |
| pegRNA | 1843 |
| pegRNA | 1844 |
| pegRNA | 1845 |
| pegRNA | 1846 |
| pegRNA | 1847 |
| pegRNA | 1848 |
| pegRNA | 1849 |
| pegRNA | 1850 |
| pegRNA | 1851 |
| pegRNA | 1852 |
| pegRNA | 1853 |
| pegRNA | 1854 |
| pegRNA | 1855 |
| pegRNA | 1856 |
| pegRNA | 1857 |
| pegRNA | 1858 |
| pegRNA | 1859 |
| pegRNA | 1860 |
| pegRNA | 1861 |
| pegRNA | 1862 |
| pegRNA | 1863 |
| pegRNA | 1864 |
| pegRNA | 1865 |
| pegRNA | 1866 |
| pegRNA | 1867 |
| pegRNA | 1868 |
| pegRNA | 1869 |
| pegRNA | 1870 |
| pegRNA | 1871 |
| pegRNA | 1872 |
| pegRNA | 1873 |
| pegRNA | 1874 |
| pegRNA | 1875 |
| pegRNA | 1876 |
| pegRNA | 1877 |
| pegRNA | 1878 |
| pegRNA | 1879 |
| pegRNA | 1880 |
| pegRNA | 1881 |
| pegRNA | 1882 |
| pegRNA | 1883 |
| pegRNA | 1884 |
| pegRNA | 1885 |
| pegRNA | 1886 |
| pegRNA | 1887 |
| pegRNA | 1888 |
| pegRNA | 1889 |
| pegRNA | 1890 |
| pegRNA | 1891 |
| pegRNA | 1892 |
| pegRNA | 1893 |
| pegRNA | 1894 |
| pegRNA | 1895 |
| pegRNA | 1896 |
| pegRNA | 1897 |
| pegRNA | 1898 |
| pegRNA | 1899 |
| pegRNA | 1900 |
| pegRNA | 1901 |
| pegRNA | 1902 |
| pegRNA | 1903 |
| pegRNA | 1904 |
| pegRNA | 1905 |
| pegRNA | 1906 |
| pegRNA | 1907 |
| pegRNA | 1908 |
| pegRNA | 1909 |
| pegRNA | 1910 |
| pegRNA | 1911 |
| pegRNA | 1912 |
| pegRNA | 1913 |
| pegRNA | 1914 |
| pegRNA | 1915 |
| pegRNA | 1916 |
| pegRNA | 1917 |
| pegRNA | 1918 |
| pegRNA | 1919 |
| pegRNA | 1920 |
| pegRNA | 1921 |
| pegRNA | 1922 |
| pegRNA | 1923 |
| pegRNA | 1924 |
| pegRNA | 1925 |
| pegRNA | 1926 |
| pegRNA | 1927 |
| pegRNA | 1928 |
| pegRNA | 1929 |
| pegRNA | 1930 |
| pegRNA | 1931 |
| pegRNA | 1932 |
| pegRNA | 1933 |
| pegRNA | 1934 |
| pegRNA | 1935 |
| pegRNA | 1936 |
| pegRNA | 1937 |
| pegRNA | 1938 |
| pegRNA | 1939 |
| pegRNA | 1940 |
| pegRNA | 1941 |
| pegRNA | 1942 |
| pegRNA | 1943 |
| pegRNA | 1944 |
| pegRNA | 1945 |
| pegRNA | 1946 |
| pegRNA | 1947 |
| pegRNA | 1948 |
| pegRNA | 1949 |
| pegRNA | 1950 |
| pegRNA | 1951 |
| pegRNA | 1952 |
| pegRNA | 1953 |
| pegRNA | 1954 |
| pegRNA | 1955 |
| pegRNA | 1956 |
| pegRNA | 1957 |
| pegRNA | 1958 |
| pegRNA | 1959 |
| pegRNA | 1960 |
| pegRNA | 1961 |
| pegRNA | 1962 |
| pegRNA | 1963 |
| pegRNA | 1964 |
| pegRNA | 1965 |
| pegRNA | 1966 |
| pegRNA | 1967 |
| pegRNA | 1968 |
| pegRNA | 1969 |
| pegRNA | 1970 |
| pegRNA | 1971 |
| pegRNA | 1972 |
| pegRNA | 1973 |
| pegRNA | 1974 |
| pegRNA | 1975 |
| pegRNA | 1976 |
| pegRNA | 1977 |
| pegRNA | 1978 |
| pegRNA | 1979 |
| pegRNA | 1980 |
| pegRNA | 1981 |
| pegRNA | 1982 |
| pegRNA | 1983 |
| pegRNA | 1984 |
| pegRNA | 1985 |
| pegRNA | 1986 |
| pegRNA | 1987 |
| pegRNA | 1988 |
| pegRNA | 1989 |
| pegRNA | 1990 |
| pegRNA | 1991 |
| pegRNA | 1992 |
| pegRNA | 1993 |
| pegRNA | 1994 |
| pegRNA | 1995 |
| pegRNA | 1996 |
| pegRNA | 1997 |
| pegRNA | 1998 |
| pegRNA | 1999 |
| pegRNA | 2000 |
| pegRNA | 2001 |
| pegRNA | 2002 |
| pegRNA | 2003 |
| pegRNA | 2004 |
| pegRNA | 2005 |
| pegRNA | 2006 |
| pegRNA | 2007 |
| pegRNA | 2008 |
| pegRNA | 2009 |
| pegRNA | 2010 |
| pegRNA | 2011 |
| pegRNA | 2012 |
| pegRNA | 2013 |
| pegRNA | 2014 |
| pegRNA | 2015 |
| pegRNA | 2016 |
| pegRNA | 2017 |
| pegRNA | 2018 |
| pegRNA | 2019 |
| pegRNA | 2020 |
| pegRNA | 2021 |
| pegRNA | 2022 |
| ngRNA | 169 |
| ngRNA | 153 |
| ngRNA | 291 |
| ngRNA | 1503 |
| ngRNA | 159 |
| ngRNA | 155 |
| ngRNA | 1502 |
| ngRNA | 160 |
| ngRNA | 681 |
| ngRNA | 158 |
| ngRNA | 161 |
| ngRNA | 168 |
| ngRNA | 157 |
| ngRNA | 166 |
| ngRNA | 167 |
| ngRNA | 164 |
| ngRNA | 162 |
| ngRNA | 154 |
| ngRNA | 165 |
| ngRNA | 290 |
| ngRNA | 163 |
| ngRNA | 156 |
| ngRNA | 1501 |
| ngRNA | 181 |
| ngRNA | 173 |
| ngRNA | 170 |
| ngRNA | 175 |
| ngRNA | 172 |
| ngRNA | 176 |
| ngRNA | 292 |
| ngRNA | 171 |
| ngRNA | 180 |
| ngRNA | 177 |
| ngRNA | 1504 |
| ngRNA | 179 |
| ngRNA | 174 |
| ngRNA | 178 |
| ngRNA | 293 |

TABLE 12

| Sequence region | SEQ ID |
|---|---|
| pegRNA Spacer | 2023 |
| PBS | 2024 |
| PBS | 2025 |
| PBS | 2026 |
| PBS | 2027 |
| PBS | 2028 |
| PBS | 2029 |
| PBS | 2030 |
| PBS | 2031 |
| PBS | 2032 |
| PBS | 2033 |
| PBS | 2034 |
| RTT | 2035 |
| RTT | 2036 |
| RTT | 2037 |
| RTT | 2038 |
| RTT | 2039 |
| RTT | 2040 |
| RTT | 2041 |
| RTT | 2042 |
| RTT | 2043 |
| RTT | 2044 |
| ngRNA Spacer | 2045 |
| ngRNA Spacer | 2046 |
| ngRNA Spacer | 2047 |
| ngRNA Spacer | 2048 |
| ngRNA Spacer | 2049 |
| ngRNA Spacer | 2050 |
| ngRNA Spacer | 2051 |
| ngRNA Spacer | 2052 |
| ngRNA Spacer | 2053 |
| ngRNA Spacer | 2054 |
| ngRNA Spacer | 2055 |
| ngRNA Spacer | 2056 |
| ngRNA Spacer | 2057 |
| ngRNA Spacer | 2058 |
| ngRNA Spacer | 2059 |
| ngRNA Spacer | 2060 |
| ngRNA Spacer | 2061 |
| ngRNA Spacer | 2062 |
| ngRNA Spacer | 2063 |
| ngRNA Spacer | 2064 |
| ngRNA Spacer | 2065 |
| ngRNA Spacer | 2066 |
| ngRNA Spacer | 2067 |
| ngRNA Spacer | 2068 |
| ngRNA Spacer | 41 |
| ngRNA Spacer | 2069 |
| ngRNA Spacer | 2070 |
| ngRNA Spacer | 2071 |
| ngRNA Spacer | 2072 |
| ngRNA Spacer | 2073 |
| ngRNA Spacer | 2074 |
| ngRNA Spacer | 2075 |
| ngRNA Spacer | 2076 |
| ngRNA Spacer | 2077 |
| ngRNA Spacer | 2078 |
| ngRNA Spacer | 2079 |
| ngRNA Spacer | 2080 |
| ngRNA Spacer | 2081 |
| ngRNA Spacer | 2082 |
| ngRNA Spacer | 2083 |
| ngRNA Spacer | 2084 |
| ngRNA Spacer | 2085 |
| ngRNA Spacer | 2086 |
| ngRNA Spacer | 2087 |
| ngRNA Spacer | 2088 |
| ngRNA Spacer | 2089 |
| ngRNA Spacer | 2090 |
| ngRNA Spacer | 2091 |
| ngRNA Spacer | 2092 |
| ngRNA Spacer | 2093 |
| ngRNA Spacer | 2094 |
| ngRNA Spacer | 2095 |
| ngRNA Spacer | 60 |
| ngRNA Spacer | 69 |
| ngRNA Spacer | 2096 |
| ngRNA Spacer | 65 |
| ngRNA Spacer | 64 |
| ngRNA Spacer | 63 |
| ngRNA Spacer | 62 |
| ngRNA Spacer | 66 |
| ngRNA Spacer | 61 |
| ngRNA Spacer | 71 |
| pegRNA | 2097 |
| pegRNA | 2098 |
| pegRNA | 2099 |
| pegRNA | 2100 |
| pegRNA | 2101 |
| pegRNA | 2102 |
| pegRNA | 2103 |
| pegRNA | 2104 |
| pegRNA | 2105 |
| pegRNA | 2106 |
| pegRNA | 2107 |
| pegRNA | 2108 |
| pegRNA | 2109 |
| pegRNA | 2110 |
| pegRNA | 2111 |
| pegRNA | 2112 |
| pegRNA | 2113 |
| pegRNA | 2114 |
| pegRNA | 2115 |
| pegRNA | 2116 |
| pegRNA | 2117 |
| pegRNA | 2118 |
| pegRNA | 2119 |
| pegRNA | 2120 |
| pegRNA | 2121 |
| pegRNA | 2122 |
| pegRNA | 2123 |
| pegRNA | 2124 |
| pegRNA | 2125 |
| pegRNA | 2127 |
| pegRNA | 2128 |
| pegRNA | 2129 |
| pegRNA | 2130 |
| pegRNA | 2131 |
| pegRNA | 2132 |
| pegRNA | 2133 |
| pegRNA | 2134 |
| pegRNA | 2135 |
| pegRNA | 2136 |
| pegRNA | 2137 |
| pegRNA | 2138 |
| pegRNA | 2139 |
| pegRNA | 2140 |
| pegRNA | 2141 |
| pegRNA | 2142 |
| pegRNA | 2143 |
| pegRNA | 2144 |
| pegRNA | 2145 |
| pegRNA | 2146 |
| pegRNA | 2147 |
| pegRNA | 2148 |
| pegRNA | 2149 |
| pegRNA | 2150 |
| pegRNA | 2151 |
| pegRNA | 2152 |
| pegRNA | 2153 |
| pegRNA | 2154 |
| pegRNA | 2155 |
| pegRNA | 2156 |
| pegRNA | 2157 |
| pegRNA | 2158 |
| pegRNA | 2159 |
| pegRNA | 2160 |
| pegRNA | 2161 |
| pegRNA | 2162 |
| pegRNA | 2163 |
| pegRNA | 2164 |
| pegRNA | 2165 |
| pegRNA | 2166 |
| pegRNA | 2167 |
| pegRNA | 2168 |
| pegRNA | 2169 |
| pegRNA | 2170 |
| pegRNA | 2171 |
| pegRNA | 2173 |
| pegRNA | 2174 |
| pegRNA | 2175 |
| pegRNA | 2176 |
| pegRNA | 2177 |
| pegRNA | 2178 |
| pegRNA | 2179 |
| pegRNA | 2180 |
| pegRNA | 2181 |
| pegRNA | 2182 |
| pegRNA | 2183 |
| pegRNA | 2184 |
| pegRNA | 2185 |
| pegRNA | 2186 |
| pegRNA | 2187 |
| pegRNA | 2188 |
| pegRNA | 2189 |
| pegRNA | 2190 |
| pegRNA | 2191 |
| pegRNA | 2192 |
| pegRNA | 2193 |
| pegRNA | 2194 |
| pegRNA | 2195 |
| pegRNA | 2196 |
| pegRNA | 2197 |
| pegRNA | 2198 |
| pegRNA | 2199 |
| pegRNA | 2200 |
| pegRNA | 2201 |
| pegRNA | 2202 |
| pegRNA | 2203 |
| pegRNA | 2204 |
| pegRNA | 2205 |
| pegRNA | 2206 |
| pegRNA | 2207 |
| pegRNA | 2208 |
| pegRNA | 2209 |
| pegRNA | 2210 |
| pegRNA | 2211 |
| pegRNA | 2212 |
| pegRNA | 2213 |
| pegRNA | 2214 |
| pegRNA | 2215 |
| pegRNA | 2216 |
| pegRNA | 2217 |
| pegRNA | 2218 |
| pegRNA | 2219 |
| pegRNA | 2220 |
| pegRNA | 2221 |
| pegRNA | 2222 |
| pegRNA | 2223 |
| pegRNA | 2224 |
| pegRNA | 2225 |
| pegRNA | 2226 |
| pegRNA | 2227 |
| pegRNA | 2228 |
| pegRNA | 2229 |
| pegRNA | 2230 |
| pegRNA | 2231 |
| pegRNA | 2232 |
| pegRNA | 2233 |
| pegRNA | 2234 |
| pegRNA | 2235 |
| pegRNA | 2236 |
| pegRNA | 2237 |
| pegRNA | 2238 |
| pegRNA | 2239 |
| pegRNA | 2240 |
| pegRNA | 2241 |
| pegRNA | 2242 |
| pegRNA | 2243 |
| pegRNA | 2244 |
| pegRNA | 2245 |
| pegRNA | 2246 |
| pegRNA | 2247 |
| pegRNA | 2248 |
| pegRNA | 2249 |
| pegRNA | 2250 |
| pegRNA | 2251 |
| pegRNA | 2252 |
| pegRNA | 2253 |
| pegRNA | 2254 |
| pegRNA | 2255 |
| pegRNA | 2256 |
| ngRNA | 2257 |
| ngRNA | 2258 |
| ngRNA | 2259 |
| ngRNA | 2260 |
| ngRNA | 2261 |
| ngRNA | 2262 |
| ngRNA | 2263 |
| ngRNA | 2264 |
| ngRNA | 2265 |
| ngRNA | 2266 |
| ngRNA | 2267 |
| ngRNA | 2268 |
| ngRNA | 2269 |
| ngRNA | 2270 |
| ngRNA | 2271 |
| ngRNA | 2272 |
| ngRNA | 2273 |
| ngRNA | 2274 |
| ngRNA | 2275 |
| ngRNA | 2276 |
| ngRNA | 2277 |
| ngRNA | 2278 |
| ngRNA | 2279 |
| ngRNA | 2280 |
| ngRNA | 2281 |
| ngRNA | 2282 |
| ngRNA | 2283 |
| ngRNA | 2284 |
| ngRNA | 2285 |
| ngRNA | 2286 |
| ngRNA | 2287 |
| ngRNA | 2288 |
| ngRNA | 2289 |
| ngRNA | 2290 |
| ngRNA | 2291 |
| ngRNA | 2292 |

TABLE 15

| Sequence region | SEQ ID |
|---|---|
| pegRNA Spacer | 2293 |
| PBS | 2294 |
| PBS | 2295 |
| PBS | 2296 |
| PBS | 2297 |
| PBS | 2298 |
| PBS | 2299 |
| PBS | 2300 |
| PBS | 2301 |
| PBS | 2302 |
| PBS | 2303 |
| PBS | 2304 |
| RTT | 2305 |
| RTT | 2306 |
| RTT | 2353 |
| RTT | 2307 |
| RTT | 2308 |
| RTT | 2309 |
| RTT | 2310 |
| RTT | 2311 |
| RTT | 2312 |
| RTT | 2313 |
| RTT | 2314 |
| RTT | 2315 |
| RTT | 2316 |
| RTT | 2317 |
| RTT | 2318 |
| RTT | 2319 |
| RTT | 2320 |
| RTT | 2321 |
| RTT | 2322 |
| RTT | 2323 |
| RTT | 2324 |
| RTT | 2325 |
| RTT | 2326 |
| RTT | 2327 |
| RTT | 2328 |
| RTT | 2329 |
| RTT | 2330 |
| RTT | 2331 |
| RTT | 2332 |
| RTT | 2333 |
| RTT | 2334 |
| RTT | 2335 |
| RTT | 2336 |
| RTT | 2337 |
| RTT | 2338 |
| RTT | 2339 |
| RTT | 2340 |
| RTT | 2341 |
| RTT | 2342 |
| RTT | 2343 |
| RTT | 2344 |
| RTT | 2345 |
| RTT | 2346 |
| RTT | 2347 |
| RTT | 2348 |
| RTT | 2349 |
| RTT | 2350 |
| RTT | 2351 |
| RTT | 2352 |
| RTT | 2354 |
| RTT | 2355 |
| RTT | 2356 |
| RTT | 2357 |
| RTT | 2358 |
| RTT | 2359 |
| RTT | 2360 |
| RTT | 2361 |
| RTT | 2362 |
| RTT | 2363 |
| RTT | 2364 |
| RTT | 2365 |
| RTT | 2366 |
| RTT | 2367 |
| RTT | 2368 |
| RTT | 2369 |
| RTT | 2370 |
| RTT | 2371 |
| RTT | 2372 |
| RTT | 2373 |
| RTT | 2374 |
| RTT | 2375 |
| RTT | 2376 |
| RTT | 2377 |
| RTT | 2378 |
| RTT | 2379 |
| RTT | 2380 |
| RTT | 2381 |
| RTT | 2382 |
| RTT | 2383 |
| RTT | 2384 |
| RTT | 2385 |
| RTT | 2386 |
| RTT | 2387 |
| RTT | 2388 |
| RTT | 2389 |
| RTT | 2390 |
| RTT | 2391 |
| RTT | 2392 |
| RTT | 2393 |
| RTT | 2394 |
| RTT | 2395 |
| RTT | 2396 |
| RTT | 2397 |
| RTT | 2398 |
| RTT | 2400 |
| RTT | 2401 |
| RTT | 2402 |
| RTT | 2403 |
| RTT | 2404 |
| RTT | 2405 |
| RTT | 2406 |
| RTT | 2407 |
| RTT | 2408 |
| RTT | 2409 |
| RTT | 2410 |
| RTT | 2411 |
| RTT | 2412 |
| RTT | 2413 |
| RTT | 2414 |
| RTT | 2415 |
| RTT | 2416 |
| RTT | 2417 |
| RTT | 2418 |
| RTT | 2419 |
| RTT | 2420 |
| RTT | 2421 |
| RTT | 2422 |
| ngRNA Spacer | 2064 |
| ngRNA Spacer | 2423 |
| ngRNA Spacer | 2424 |
| ngRNA Spacer | 2425 |
| ngRNA Spacer | 2426 |
| ngRNA Spacer | 2427 |
| ngRNA Spacer | 2428 |
| ngRNA Spacer | 2429 |
| ngRNA Spacer | 2430 |
| ngRNA Spacer | 2431 |
| ngRNA Spacer | 2432 |
| ngRNA Spacer | 2433 |
| ngRNA Spacer | 2434 |
| ngRNA Spacer | 2435 |
| ngRNA Spacer | 2091 |
| ngRNA Spacer | 2060 |
| ngRNA Spacer | 2436 |
| ngRNA Spacer | 2437 |
| ngRNA Spacer | 2054 |
| ngRNA Spacer | 2070 |
| ngRNA Spacer | 2078 |
| ngRNA Spacer | 2046 |
| ngRNA Spacer | 2047 |
| ngRNA Spacer | 2050 |
| ngRNA Spacer | 2051 |
| ngRNA Spacer | 2057 |
| ngRNA Spacer | 2058 |
| ngRNA Spacer | 2061 |
| ngRNA Spacer | 2438 |
| ngRNA Spacer | 2080 |
| ngRNA Spacer | 71 |
| ngRNA Spacer | 61 |
| ngRNA Spacer | 66 |
| ngRNA Spacer | 2074 |
| ngRNA Spacer | 62 |
| ngRNA Spacer | 64 |
| ngRNA Spacer | 65 |
| ngRNA Spacer | 69 |
| ngRNA Spacer | 60 |
| ngRNA Spacer | 2083 |
| ngRNA Spacer | 2066 |
| ngRNA Spacer | 2056 |
| ngRNA Spacer | 2062 |
| ngRNA Spacer | 2089 |
| ngRNA Spacer | 2439 |
| ngRNA Spacer | 2090 |
| ngRNA Spacer | 2059 |
| ngRNA Spacer | 2081 |
| ngRNA Spacer | 2069 |
| ngRNA Spacer | 63 |
| ngRNA Spacer | 2088 |
| ngRNA Spacer | 2068 |
| ngRNA Spacer | 2440 |
| ngRNA Spacer | 2072 |
| ngRNA Spacer | 2079 |
| ngRNA Spacer | 2073 |
| ngRNA Spacer | 2077 |
| ngRNA Spacer | 2093 |
| ngRNA Spacer | 2071 |
| ngRNA Spacer | 2048 |
| ngRNA Spacer | 2049 |
| ngRNA Spacer | 41 |
| ngRNA Spacer | 2085 |
| ngRNA Spacer | 2087 |
| ngRNA Spacer | 2441 |
| ngRNA Spacer | 2045 |
| ngRNA Spacer | 2063 |
| ngRNA Spacer | 2084 |
| ngRNA Spacer | 2076 |
| ngRNA Spacer | 2095 |
| ngRNA Spacer | 2075 |
| ngRNA Spacer | 2442 |
| ngRNA Spacer | 2443 |
| ngRNA Spacer | 2092 |
| ngRNA Spacer | 2055 |
| ngRNA Spacer | 2052 |
| ngRNA Spacer | 2086 |
| ngRNA Spacer | 2444 |
| ngRNA Spacer | 2082 |
| ngRNA Spacer | 2067 |
| ngRNA Spacer | 2096 |
| ngRNA Spacer | 2094 |
| ngRNA Spacer | 2053 |
| pegRNA | 2445 |
| pegRNA | 2446 |
| pegRNA | 2447 |
| pegRNA | 2448 |
| pegRNA | 2449 |
| pegRNA | 2450 |
| pegRNA | 2451 |
| pegRNA | 2452 |
| pegRNA | 2453 |
| pegRNA | 2454 |
| pegRNA | 2455 |
| pegRNA | 2456 |
| pegRNA | 2457 |
| pegRNA | 2458 |
| pegRNA | 2459 |
| pegRNA | 2460 |
| pegRNA | 2461 |
| pegRNA | 2462 |
| pegRNA | 2463 |
| pegRNA | 2464 |
| pegRNA | 2465 |
| pegRNA | 2466 |
| pegRNA | 2467 |
| pegRNA | 2468 |
| pegRNA | 2469 |
| pegRNA | 2470 |
| pegRNA | 2471 |
| pegRNA | 2472 |
| pegRNA | 2473 |
| pegRNA | 2474 |
| pegRNA | 2475 |
| pegRNA | 2476 |
| pegRNA | 2477 |
| pegRNA | 2478 |
| pegRNA | 2479 |
| pegRNA | 2480 |
| pegRNA | 2481 |
| pegRNA | 2482 |
| pegRNA | 2483 |
| pegRNA | 2484 |
| pegRNA | 2485 |
| pegRNA | 2486 |
| pegRNA | 2487 |
| pegRNA | 2488 |
| pegRNA | 2489 |
| pegRNA | 2490 |
| pegRNA | 2491 |
| pegRNA | 2492 |
| pegRNA | 2493 |
| pegRNA | 2494 |
| pegRNA | 2495 |
| pegRNA | 2496 |
| pegRNA | 2497 |
| pegRNA | 2498 |
| pegRNA | 2499 |
| pegRNA | 2500 |
| pegRNA | 2501 |
| pegRNA | 2502 |
| pegRNA | 2503 |
| pegRNA | 2504 |
| pegRNA | 2505 |
| pegRNA | 2506 |
| pegRNA | 2507 |
| pegRNA | 2508 |
| pegRNA | 2509 |
| pegRNA | 2510 |
| pegRNA | 2511 |
| pegRNA | 2512 |
| pegRNA | 2513 |
| pegRNA | 2514 |
| pegRNA | 2515 |
| pegRNA | 2516 |
| pegRNA | 2517 |
| pegRNA | 2518 |
| pegRNA | 2519 |
| pegRNA | 2520 |
| pegRNA | 2521 |
| pegRNA | 2522 |
| pegRNA | 2523 |
| pegRNA | 2524 |
| pegRNA | 2525 |
| pegRNA | 2526 |
| pegRNA | 2527 |
| pegRNA | 2528 |
| pegRNA | 2529 |
| pegRNA | 2530 |
| pegRNA | 2531 |
| pegRNA | 2532 |
| pegRNA | 2533 |
| pegRNA | 2534 |
| pegRNA | 2535 |
| pegRNA | 2536 |
| pegRNA | 2537 |
| pegRNA | 2538 |
| pegRNA | 2539 |
| pegRNA | 2540 |
| pegRNA | 2541 |
| pegRNA | 2542 |
| pegRNA | 2543 |
| pegRNA | 2544 |
| pegRNA | 2545 |
| pegRNA | 2546 |
| pegRNA | 2547 |
| pegRNA | 2548 |
| pegRNA | 2549 |
| pegRNA | 2550 |
| pegRNA | 2551 |
| pegRNA | 2552 |
| pegRNA | 2553 |
| pegRNA | 2554 |
| pegRNA | 2555 |
| pegRNA | 2556 |
| pegRNA | 2557 |
| pegRNA | 2558 |
| pegRNA | 2559 |
| pegRNA | 2560 |
| pegRNA | 2561 |
| pegRNA | 2562 |
| pegRNA | 2563 |
| pegRNA | 2564 |
| pegRNA | 2565 |
| pegRNA | 2566 |
| pegRNA | 2567 |
| pegRNA | 2568 |
| pegRNA | 2569 |
| pegRNA | 2570 |
| pegRNA | 2571 |
| pegRNA | 2572 |
| pegRNA | 2573 |
| pegRNA | 2574 |
| pegRNA | 2575 |
| pegRNA | 2576 |
| pegRNA | 2577 |
| pegRNA | 2578 |
| pegRNA | 2579 |
| pegRNA | 2580 |
| pegRNA | 2581 |
| pegRNA | 2582 |
| pegRNA | 2583 |
| pegRNA | 2584 |
| pegRNA | 2585 |
| pegRNA | 2586 |
| pegRNA | 2587 |
| pegRNA | 2588 |
| pegRNA | 2589 |
| pegRNA | 2590 |
| pegRNA | 2591 |
| pegRNA | 2592 |
| pegRNA | 2593 |
| pegRNA | 2594 |
| pegRNA | 2595 |
| pegRNA | 2596 |
| pegRNA | 2597 |
| pegRNA | 2598 |
| pegRNA | 2599 |
| pegRNA | 2600 |
| pegRNA | 2601 |
| pegRNA | 2602 |
| pegRNA | 2603 |
| pegRNA | 2604 |
| pegRNA | 2605 |
| pegRNA | 2606 |
| pegRNA | 2607 |
| pegRNA | 2608 |
| pegRNA | 2609 |
| pegRNA | 2610 |
| pegRNA | 2611 |
| pegRNA | 2612 |
| pegRNA | 2613 |
| pegRNA | 2614 |
| pegRNA | 2615 |
| pegRNA | 2616 |
| pegRNA | 2617 |
| pegRNA | 2618 |
| pegRNA | 2619 |
| pegRNA | 2620 |
| pegRNA | 2621 |
| pegRNA | 2622 |
| pegRNA | 2623 |
| pegRNA | 2624 |
| pegRNA | 2625 |
| pegRNA | 2626 |
| pegRNA | 2627 |
| pegRNA | 2628 |
| pegRNA | 2629 |
| pegRNA | 2630 |
| pegRNA | 2631 |
| pegRNA | 2632 |
| pegRNA | 2633 |
| pegRNA | 2634 |
| pegRNA | 2635 |
| pegRNA | 2636 |
| pegRNA | 2637 |
| pegRNA | 2638 |
| pegRNA | 2639 |
| pegRNA | 2640 |
| pegRNA | 2641 |
| pegRNA | 2642 |
| pegRNA | 2643 |
| pegRNA | 2644 |
| pegRNA | 2645 |
| pegRNA | 2646 |
| pegRNA | 2647 |
| pegRNA | 2648 |
| pegRNA | 2649 |
| pegRNA | 2650 |
| pegRNA | 2651 |
| pegRNA | 2652 |
| pegRNA | 2653 |
| pegRNA | 2654 |
| pegRNA | 2655 |
| pegRNA | 2656 |
| pegRNA | 2657 |
| pegRNA | 2658 |
| pegRNA | 2659 |
| pegRNA | 2660 |
| pegRNA | 2661 |
| pegRNA | 2662 |
| pegRNA | 2663 |
| pegRNA | 2664 |
| pegRNA | 2665 |
| pegRNA | 2666 |
| pegRNA | 2667 |
| pegRNA | 2668 |
| pegRNA | 2669 |
| pegRNA | 2670 |
| pegRNA | 2671 |
| pegRNA | 2672 |
| pegRNA | 2673 |
| pegRNA | 2674 |
| pegRNA | 2675 |
| pegRNA | 2676 |
| pegRNA | 2677 |
| pegRNA | 2678 |
| pegRNA | 2679 |
| pegRNA | 2680 |
| pegRNA | 2681 |
| pegRNA | 2682 |
| pegRNA | 2683 |
| pegRNA | 2684 |
| pegRNA | 2685 |
| pegRNA | 2686 |
| pegRNA | 2687 |
| pegRNA | 2688 |
| pegRNA | 2689 |
| pegRNA | 2690 |
| pegRNA | 2691 |
| pegRNA | 2692 |
| pegRNA | 2693 |
| pegRNA | 2694 |
| pegRNA | 2695 |
| pegRNA | 2696 |
| pegRNA | 2697 |
| pegRNA | 2698 |
| pegRNA | 2699 |
| pegRNA | 2700 |
| pegRNA | 2701 |
| pegRNA | 2702 |
| pegRNA | 2703 |
| pegRNA | 2704 |
| pegRNA | 2705 |
| pegRNA | 2706 |
| pegRNA | 2707 |
| pegRNA | 2708 |
| pegRNA | 2709 |
| pegRNA | 2710 |
| pegRNA | 2711 |
| pegRNA | 2712 |
| pegRNA | 2713 |
| pegRNA | 2714 |
| pegRNA | 2715 |
| pegRNA | 2716 |
| pegRNA | 2717 |
| pegRNA | 2718 |
| pegRNA | 2719 |
| pegRNA | 2720 |
| pegRNA | 2721 |
| pegRNA | 2722 |
| pegRNA | 2723 |
| pegRNA | 2724 |
| pegRNA | 2725 |
| pegRNA | 2726 |
| pegRNA | 2727 |
| pegRNA | 2728 |
| pegRNA | 2729 |
| pegRNA | 2730 |
| pegRNA | 2731 |
| pegRNA | 2732 |
| pegRNA | 2733 |
| pegRNA | 2734 |
| pegRNA | 2735 |
| pegRNA | 2736 |
| pegRNA | 2737 |
| pegRNA | 2738 |
| pegRNA | 2739 |
| pegRNA | 2740 |
| pegRNA | 2741 |
| pegRNA | 2742 |
| pegRNA | 2743 |
| pegRNA | 2744 |
| pegRNA | 2745 |
| pegRNA | 2746 |
| pegRNA | 2747 |
| pegRNA | 2748 |
| pegRNA | 2749 |
| pegRNA | 2750 |
| pegRNA | 2751 |
| pegRNA | 2752 |
| pegRNA | 2753 |
| pegRNA | 2754 |
| pegRNA | 2755 |
| pegRNA | 2756 |
| pegRNA | 2757 |
| pegRNA | 2758 |
| pegRNA | 2759 |
| pegRNA | 2760 |
| pegRNA | 2761 |
| pegRNA | 2762 |
| pegRNA | 2763 |
| pegRNA | 2764 |
| pegRNA | 2765 |
| pegRNA | 2766 |
| pegRNA | 2767 |
| pegRNA | 2768 |
| pegRNA | 2769 |
| pegRNA | 2770 |
| pegRNA | 2771 |
| pegRNA | 2772 |
| pegRNA | 2773 |
| pegRNA | 2774 |
| pegRNA | 2775 |
| pegRNA | 2776 |
| pegRNA | 2777 |
| pegRNA | 2778 |
| pegRNA | 2779 |
| pegRNA | 2780 |
| pegRNA | 2781 |
| pegRNA | 2782 |
| pegRNA | 2783 |
| pegRNA | 2784 |
| pegRNA | 2785 |
| pegRNA | 2786 |
| pegRNA | 2787 |
| pegRNA | 2788 |
| pegRNA | 2789 |
| pegRNA | 2790 |
| pegRNA | 2791 |
| pegRNA | 2792 |
| pegRNA | 2793 |
| pegRNA | 2794 |
| pegRNA | 2795 |
| pegRNA | 2796 |
| pegRNA | 2797 |
| pegRNA | 2798 |
| pegRNA | 2799 |
| pegRNA | 2800 |
| pegRNA | 2801 |
| pegRNA | 2802 |
| pegRNA | 2803 |
| pegRNA | 2804 |
| pegRNA | 2805 |
| pegRNA | 2806 |
| pegRNA | 2807 |
| pegRNA | 2808 |
| pegRNA | 2809 |
| pegRNA | 2810 |
| pegRNA | 2811 |
| pegRNA | 2812 |
| pegRNA | 2813 |
| pegRNA | 2814 |
| pegRNA | 2815 |
| pegRNA | 2816 |
| pegRNA | 2817 |
| pegRNA | 2818 |
| pegRNA | 2819 |
| pegRNA | 2820 |
| pegRNA | 2821 |
| pegRNA | 2822 |
| pegRNA | 2823 |
| pegRNA | 2824 |
| pegRNA | 2825 |
| pegRNA | 2826 |
| pegRNA | 2827 |
| pegRNA | 2828 |
| pegRNA | 2829 |
| pegRNA | 2830 |
| pegRNA | 2831 |
| pegRNA | 2832 |
| pegRNA | 2833 |
| pegRNA | 2834 |
| pegRNA | 2835 |
| pegRNA | 2836 |
| pegRNA | 2837 |
| pegRNA | 2838 |
| pegRNA | 2839 |
| pegRNA | 2840 |
| pegRNA | 2841 |
| pegRNA | 2842 |
| pegRNA | 2843 |
| pegRNA | 2844 |
| pegRNA | 2845 |
| pegRNA | 2846 |
| pegRNA | 2847 |
| pegRNA | 2848 |
| pegRNA | 2849 |
| pegRNA | 2850 |
| pegRNA | 2851 |
| pegRNA | 2852 |
| pegRNA | 2853 |
| pegRNA | 2854 |
| pegRNA | 2855 |
| pegRNA | 2856 |
| pegRNA | 2857 |
| pegRNA | 2858 |
| pegRNA | 2859 |
| pegRNA | 2860 |
| pegRNA | 2861 |
| pegRNA | 2862 |
| pegRNA | 2863 |
| pegRNA | 2864 |
| pegRNA | 2865 |
| pegRNA | 2866 |
| pegRNA | 2867 |
| pegRNA | 2868 |
| pegRNA | 2869 |
| pegRNA | 2870 |
| pegRNA | 2871 |
| pegRNA | 2872 |
| pegRNA | 2873 |
| pegRNA | 2874 |
| pegRNA | 2875 |
| pegRNA | 2876 |
| pegRNA | 2877 |
| pegRNA | 2878 |
| pegRNA | 2879 |
| pegRNA | 2880 |
| pegRNA | 2881 |
| pegRNA | 2882 |
| pegRNA | 2883 |
| pegRNA | 2884 |
| pegRNA | 2885 |
| pegRNA | 2886 |
| pegRNA | 2887 |
| pegRNA | 2888 |
| pegRNA | 2889 |
| pegRNA | 2890 |
| pegRNA | 2891 |
| pegRNA | 2892 |
| pegRNA | 2893 |
| pegRNA | 2894 |
| pegRNA | 2895 |
| pegRNA | 2896 |
| pegRNA | 2897 |
| pegRNA | 2898 |
| pegRNA | 2899 |
| pegRNA | 2900 |
| pegRNA | 2901 |
| pegRNA | 2902 |
| pegRNA | 2903 |
| pegRNA | 2904 |
| pegRNA | 2905 |
| pegRNA | 2906 |
| pegRNA | 2907 |
| pegRNA | 2908 |
| pegRNA | 2909 |
| pegRNA | 2910 |
| pegRNA | 2911 |
| pegRNA | 2912 |
| pegRNA | 2913 |
| pegRNA | 2914 |
| pegRNA | 2915 |
| pegRNA | 2916 |
| pegRNA | 2917 |
| pegRNA | 2918 |
| pegRNA | 2919 |
| pegRNA | 2920 |
| pegRNA | 2921 |
| pegRNA | 2922 |
| pegRNA | 2923 |
| pegRNA | 2924 |
| pegRNA | 2925 |
| pegRNA | 2926 |
| pegRNA | 2927 |
| pegRNA | 2928 |
| pegRNA | 2929 |
| pegRNA | 2930 |
| pegRNA | 2931 |
| pegRNA | 2932 |
| pegRNA | 2933 |
| pegRNA | 2934 |
| pegRNA | 2935 |
| pegRNA | 2936 |
| pegRNA | 2937 |
| pegRNA | 2938 |
| pegRNA | 2939 |
| pegRNA | 2940 |
| pegRNA | 2941 |
| pegRNA | 2942 |
| pegRNA | 2943 |
| pegRNA | 2944 |
| pegRNA | 2945 |
| pegRNA | 2946 |
| pegRNA | 2947 |
| pegRNA | 2948 |
| pegRNA | 2949 |
| pegRNA | 2950 |
| pegRNA | 2951 |
| pegRNA | 2952 |
| pegRNA | 2953 |
| pegRNA | 2954 |
| pegRNA | 2955 |
| pegRNA | 2956 |
| pegRNA | 2957 |
| pegRNA | 2958 |
| pegRNA | 2959 |
| pegRNA | 2960 |
| pegRNA | 2961 |
| pegRNA | 2962 |
| pegRNA | 2963 |
| pegRNA | 2964 |
| pegRNA | 2965 |
| pegRNA | 2966 |
| pegRNA | 2967 |
| pegRNA | 2968 |
| pegRNA | 2969 |
| pegRNA | 2970 |
| pegRNA | 2971 |
| pegRNA | 2972 |
| pegRNA | 2973 |
| pegRNA | 2974 |
| pegRNA | 2975 |
| pegRNA | 2976 |
| pegRNA | 2977 |
| pegRNA | 2978 |
| pegRNA | 2979 |
| pegRNA | 2980 |
| pegRNA | 2981 |
| pegRNA | 2982 |
| pegRNA | 2983 |
| pegRNA | 2984 |
| pegRNA | 2985 |
| pegRNA | 2986 |
| pegRNA | 2987 |
| pegRNA | 2988 |
| pegRNA | 2989 |
| pegRNA | 2990 |
| pegRNA | 2991 |
| pegRNA | 2992 |
| pegRNA | 2993 |
| pegRNA | 2994 |
| pegRNA | 2995 |
| pegRNA | 2996 |
| pegRNA | 2997 |
| pegRNA | 2998 |
| pegRNA | 2999 |
| pegRNA | 3000 |
| pegRNA | 3001 |
| pegRNA | 3002 |
| pegRNA | 3003 |
| pegRNA | 3004 |
| pegRNA | 3005 |
| pegRNA | 3006 |
| pegRNA | 3007 |
| pegRNA | 3008 |
| pegRNA | 3009 |
| pegRNA | 3010 |
| pegRNA | 3011 |
| pegRNA | 3012 |
| pegRNA | 3013 |
| pegRNA | 3014 |
| pegRNA | 3015 |
| pegRNA | 3016 |
| pegRNA | 3017 |
| pegRNA | 3018 |
| pegRNA | 3019 |
| pegRNA | 3020 |
| pegRNA | 3021 |
| pegRNA | 3022 |
| pegRNA | 3023 |
| pegRNA | 3024 |
| pegRNA | 3025 |
| pegRNA | 3026 |
| pegRNA | 3027 |
| pegRNA | 3028 |
| pegRNA | 3029 |
| pegRNA | 3030 |
| pegRNA | 3031 |
| pegRNA | 3032 |
| pegRNA | 3033 |
| pegRNA | 3034 |
| pegRNA | 3035 |
| pegRNA | 3036 |
| pegRNA | 3037 |
| pegRNA | 3038 |
| pegRNA | 3039 |
| pegRNA | 3040 |
| pegRNA | 3041 |
| pegRNA | 3042 |
| pegRNA | 3043 |
| pegRNA | 3044 |
| pegRNA | 3045 |
| pegRNA | 3046 |
| pegRNA | 3047 |
| pegRNA | 3048 |
| pegRNA | 3049 |
| pegRNA | 3050 |
| pegRNA | 3051 |
| pegRNA | 3052 |
| pegRNA | 3053 |
| pegRNA | 3054 |
| pegRNA | 3055 |
| pegRNA | 3056 |
| pegRNA | 3057 |
| pegRNA | 3058 |
| pegRNA | 3059 |
| pegRNA | 3060 |
| pegRNA | 3061 |
| pegRNA | 3062 |
| pegRNA | 3063 |
| pegRNA | 3064 |
| pegRNA | 3065 |
| pegRNA | 3066 |
| pegRNA | 3067 |
| pegRNA | 3068 |
| pegRNA | 3069 |
| pegRNA | 3070 |
| pegRNA | 3071 |
| pegRNA | 3072 |
| pegRNA | 3073 |
| pegRNA | 3074 |
| pegRNA | 3075 |
| pegRNA | 3076 |
| pegRNA | 3077 |
| pegRNA | 3078 |
| pegRNA | 3079 |
| pegRNA | 3080 |
| pegRNA | 3081 |
| pegRNA | 3082 |
| pegRNA | 3083 |
| pegRNA | 3084 |
| pegRNA | 3085 |
| pegRNA | 3086 |
| pegRNA | 3087 |
| pegRNA | 3088 |
| pegRNA | 3089 |
| pegRNA | 3090 |
| pegRNA | 3091 |
| pegRNA | 3092 |
| pegRNA | 3093 |
| pegRNA | 3094 |
| pegRNA | 3095 |
| pegRNA | 3096 |
| pegRNA | 3097 |
| pegRNA | 3098 |
| pegRNA | 3099 |
| pegRNA | 3100 |
| pegRNA | 3101 |
| pegRNA | 3102 |
| pegRNA | 3103 |
| pegRNA | 3104 |
| pegRNA | 3105 |
| pegRNA | 3106 |
| pegRNA | 3107 |
| pegRNA | 3108 |
| pegRNA | 3109 |
| pegRNA | 3110 |
| pegRNA | 3111 |
| pegRNA | 3112 |
| pegRNA | 3113 |
| pegRNA | 3114 |
| pegRNA | 3115 |
| pegRNA | 3116 |
| pegRNA | 3117 |
| pegRNA | 3118 |
| pegRNA | 3119 |
| pegRNA | 3120 |
| pegRNA | 3121 |
| pegRNA | 3122 |
| pegRNA | 3123 |
| pegRNA | 3124 |
| pegRNA | 3125 |
| pegRNA | 3126 |
| pegRNA | 3127 |
| pegRNA | 3128 |
| pegRNA | 3129 |
| pegRNA | 3130 |
| pegRNA | 3131 |
| pegRNA | 3132 |
| pegRNA | 3133 |
| pegRNA | 3134 |
| pegRNA | 3135 |
| pegRNA | 3136 |
| pegRNA | 3137 |
| pegRNA | 3138 |
| pegRNA | 3139 |
| pegRNA | 3140 |
| pegRNA | 3141 |
| pegRNA | 3142 |
| pegRNA | 3143 |
| pegRNA | 3144 |
| pegRNA | 3145 |
| pegRNA | 3146 |
| pegRNA | 3147 |
| pegRNA | 3148 |
| pegRNA | 3149 |
| pegRNA | 3150 |
| pegRNA | 3151 |
| pegRNA | 3152 |
| pegRNA | 3153 |
| pegRNA | 3154 |
| pegRNA | 3155 |
| pegRNA | 3156 |
| pegRNA | 3157 |
| pegRNA | 3158 |
| pegRNA | 3159 |
| pegRNA | 3160 |
| pegRNA | 3161 |
| pegRNA | 3162 |
| pegRNA | 3163 |
| pegRNA | 3164 |
| pegRNA | 3165 |
| pegRNA | 3166 |
| pegRNA | 3167 |
| pegRNA | 3168 |
| pegRNA | 3169 |
| pegRNA | 3170 |
| pegRNA | 3171 |
| pegRNA | 3172 |
| pegRNA | 3173 |
| pegRNA | 3174 |
| pegRNA | 3175 |
| pegRNA | 3176 |
| pegRNA | 3177 |
| pegRNA | 3178 |
| pegRNA | 3179 |
| pegRNA | 3180 |
| pegRNA | 3181 |
| pegRNA | 3182 |
| pegRNA | 3183 |
| pegRNA | 3184 |
| pegRNA | 3185 |
| pegRNA | 3186 |
| pegRNA | 3187 |
| pegRNA | 3188 |
| pegRNA | 3189 |
| pegRNA | 3190 |
| pegRNA | 3191 |
| pegRNA | 3192 |
| pegRNA | 3193 |
| pegRNA | 3194 |
| pegRNA | 3195 |
| pegRNA | 3196 |
| pegRNA | 3197 |
| pegRNA | 3198 |
| pegRNA | 3199 |
| pegRNA | 3200 |
| pegRNA | 3201 |
| pegRNA | 3202 |
| pegRNA | 3203 |
| pegRNA | 3204 |
| pegRNA | 3205 |
| pegRNA | 3206 |
| pegRNA | 3207 |
| pegRNA | 3208 |
| pegRNA | 3209 |
| pegRNA | 3210 |
| pegRNA | 3211 |
| pegRNA | 3212 |
| pegRNA | 3213 |
| pegRNA | 3214 |
| pegRNA | 3215 |
| pegRNA | 3216 |
| pegRNA | 3217 |
| pegRNA | 3218 |
| pegRNA | 3219 |
| pegRNA | 3220 |
| pegRNA | 3221 |
| pegRNA | 3222 |
| pegRNA | 3223 |
| pegRNA | 3224 |
| pegRNA | 3225 |
| pegRNA | 3226 |
| pegRNA | 3227 |
| pegRNA | 3228 |
| pegRNA | 3229 |
| pegRNA | 3230 |
| pegRNA | 3231 |
| pegRNA | 3232 |
| pegRNA | 3233 |
| pegRNA | 3234 |
| pegRNA | 3235 |
| pegRNA | 3236 |
| pegRNA | 3237 |
| pegRNA | 3238 |
| pegRNA | 3239 |
| pegRNA | 3240 |
| pegRNA | 3241 |
| pegRNA | 3242 |
| pegRNA | 3243 |
| pegRNA | 3244 |
| pegRNA | 3245 |
| pegRNA | 3246 |
| pegRNA | 3247 |
| pegRNA | 3248 |
| pegRNA | 3249 |
| pegRNA | 3250 |
| pegRNA | 3251 |
| pegRNA | 3252 |
| pegRNA | 3253 |
| pegRNA | 3254 |
| pegRNA | 3255 |
| pegRNA | 3256 |
| pegRNA | 3257 |
| pegRNA | 3258 |
| pegRNA | 3259 |
| pegRNA | 3260 |
| pegRNA | 3261 |
| pegRNA | 3262 |
| pegRNA | 3263 |
| pegRNA | 3264 |
| pegRNA | 3265 |
| pegRNA | 3266 |
| pegRNA | 3267 |
| pegRNA | 3268 |
| pegRNA | 3269 |
| pegRNA | 3270 |
| pegRNA | 3271 |
| pegRNA | 3272 |
| pegRNA | 3273 |
| pegRNA | 3274 |
| pegRNA | 3275 |
| pegRNA | 3276 |
| pegRNA | 3277 |
| pegRNA | 3278 |
| pegRNA | 3279 |
| pegRNA | 3280 |
| pegRNA | 3281 |
| pegRNA | 3282 |
| pegRNA | 3283 |
| pegRNA | 3284 |
| pegRNA | 3285 |
| pegRNA | 3286 |
| pegRNA | 3287 |
| pegRNA | 3288 |
| pegRNA | 3289 |
| pegRNA | 3290 |
| pegRNA | 3291 |
| pegRNA | 3292 |
| pegRNA | 3293 |
| pegRNA | 3294 |
| pegRNA | 3295 |
| pegRNA | 3296 |
| pegRNA | 3297 |
| pegRNA | 3298 |
| pegRNA | 3299 |
| pegRNA | 3300 |
| pegRNA | 3301 |
| pegRNA | 3302 |
| pegRNA | 3303 |
| pegRNA | 3304 |
| pegRNA | 3305 |
| pegRNA | 3306 |
| pegRNA | 3307 |
| pegRNA | 3308 |
| pegRNA | 3309 |
| pegRNA | 3310 |
| pegRNA | 3311 |
| pegRNA | 3312 |
| pegRNA | 3313 |
| pegRNA | 3314 |
| pegRNA | 3315 |
| pegRNA | 3316 |
| pegRNA | 3317 |
| pegRNA | 3318 |
| pegRNA | 3319 |
| pegRNA | 3320 |
| pegRNA | 3321 |
| pegRNA | 3322 |
| pegRNA | 3323 |
| pegRNA | 3324 |
| pegRNA | 3325 |
| pegRNA | 3326 |
| pegRNA | 3327 |
| pegRNA | 3328 |
| pegRNA | 3329 |
| pegRNA | 3330 |
| pegRNA | 3331 |
| pegRNA | 3332 |
| pegRNA | 3333 |
| pegRNA | 3334 |
| pegRNA | 3335 |
| pegRNA | 3336 |
| pegRNA | 3337 |
| pegRNA | 3338 |
| pegRNA | 3339 |
| pegRNA | 3340 |
| pegRNA | 3341 |
| pegRNA | 3342 |
| pegRNA | 3343 |
| pegRNA | 3344 |
| pegRNA | 3345 |
| pegRNA | 3346 |
| pegRNA | 3347 |
| pegRNA | 3348 |
| pegRNA | 3349 |
| pegRNA | 3350 |
| pegRNA | 3351 |
| pegRNA | 3352 |
| pegRNA | 3353 |
| pegRNA | 3354 |
| pegRNA | 3355 |
| pegRNA | 3356 |
| pegRNA | 3357 |
| pegRNA | 3358 |
| pegRNA | 3359 |
| pegRNA | 3360 |
| pegRNA | 3361 |
| pegRNA | 3362 |
| pegRNA | 3363 |
| pegRNA | 3364 |
| pegRNA | 3365 |
| pegRNA | 3366 |
| pegRNA | 3367 |
| pegRNA | 3368 |
| pegRNA | 3369 |
| pegRNA | 3370 |
| pegRNA | 3371 |
| pegRNA | 3372 |
| pegRNA | 3373 |
| pegRNA | 3374 |
| pegRNA | 3375 |
| pegRNA | 3376 |
| pegRNA | 3377 |
| pegRNA | 3378 |
| pegRNA | 3379 |
| pegRNA | 3380 |
| pegRNA | 3381 |
| pegRNA | 3382 |
| pegRNA | 3383 |
| pegRNA | 3384 |
| pegRNA | 3385 |
| pegRNA | 3386 |
| pegRNA | 3387 |
| pegRNA | 3388 |
| pegRNA | 3389 |
| pegRNA | 3390 |
| pegRNA | 3391 |
| pegRNA | 3392 |
| pegRNA | 3393 |
| pegRNA | 3394 |
| pegRNA | 3395 |
| pegRNA | 3396 |
| pegRNA | 3397 |
| pegRNA | 3398 |
| pegRNA | 3399 |
| pegRNA | 3400 |
| pegRNA | 3401 |
| pegRNA | 3402 |
| pegRNA | 3403 |
| pegRNA | 3404 |
| pegRNA | 3405 |
| pegRNA | 3406 |
| pegRNA | 3407 |
| pegRNA | 3408 |
| pegRNA | 3409 |
| pegRNA | 3410 |
| pegRNA | 3411 |
| pegRNA | 3412 |
| pegRNA | 3413 |
| pegRNA | 3414 |
| pegRNA | 3415 |
| pegRNA | 3416 |
| pegRNA | 3417 |
| pegRNA | 3418 |
| pegRNA | 3419 |
| pegRNA | 3420 |
| pegRNA | 3421 |
| pegRNA | 3422 |
| pegRNA | 3423 |
| pegRNA | 3424 |
| pegRNA | 3425 |
| pegRNA | 3426 |
| pegRNA | 3427 |
| pegRNA | 3428 |
| pegRNA | 3429 |
| pegRNA | 3430 |
| pegRNA | 3431 |
| pegRNA | 3432 |
| pegRNA | 3433 |
| pegRNA | 3434 |
| pegRNA | 3435 |
| pegRNA | 3436 |
| pegRNA | 3437 |
| pegRNA | 3438 |
| pegRNA | 3439 |
| pegRNA | 3440 |
| pegRNA | 3441 |
| pegRNA | 3442 |
| pegRNA | 3443 |
| pegRNA | 3444 |
| pegRNA | 3445 |
| pegRNA | 3446 |
| pegRNA | 3447 |
| pegRNA | 3448 |
| pegRNA | 3449 |
| pegRNA | 3450 |
| pegRNA | 3451 |
| pegRNA | 3452 |
| pegRNA | 3453 |
| pegRNA | 3454 |
| pegRNA | 3455 |
| pegRNA | 3456 |
| pegRNA | 3457 |
| pegRNA | 3458 |
| pegRNA | 3459 |
| pegRNA | 3460 |
| pegRNA | 3461 |
| pegRNA | 3462 |
| pegRNA | 3463 |
| pegRNA | 3464 |
| pegRNA | 3465 |
| pegRNA | 3466 |
| pegRNA | 3467 |
| pegRNA | 3468 |
| pegRNA | 3469 |
| pegRNA | 3470 |
| pegRNA | 3471 |
| pegRNA | 3472 |
| pegRNA | 3473 |
| pegRNA | 3474 |
| pegRNA | 3475 |
| pegRNA | 3476 |
| pegRNA | 3477 |
| pegRNA | 3478 |
| pegRNA | 3479 |
| pegRNA | 3480 |
| pegRNA | 3481 |
| pegRNA | 3482 |
| pegRNA | 3483 |
| pegRNA | 3484 |
| pegRNA | 3485 |
| pegRNA | 3486 |
| pegRNA | 3487 |
| pegRNA | 3488 |
| pegRNA | 3489 |
| pegRNA | 3490 |
| pegRNA | 3491 |
| pegRNA | 3492 |
| pegRNA | 3493 |
| pegRNA | 3494 |
| pegRNA | 3495 |
| pegRNA | 3496 |
| pegRNA | 3497 |
| pegRNA | 3498 |
| pegRNA | 3499 |
| pegRNA | 3500 |
| pegRNA | 3501 |
| pegRNA | 3502 |
| pegRNA | 3503 |
| pegRNA | 3504 |
| pegRNA | 3505 |
| pegRNA | 3506 |
| pegRNA | 3507 |
| pegRNA | 3508 |
| pegRNA | 3509 |
| pegRNA | 3510 |
| pegRNA | 3511 |
| pegRNA | 3512 |
| pegRNA | 3513 |
| pegRNA | 3514 |
| pegRNA | 3515 |
| pegRNA | 3516 |
| pegRNA | 3517 |
| pegRNA | 3518 |
| pegRNA | 3519 |
| pegRNA | 3520 |
| pegRNA | 3521 |
| pegRNA | 3522 |
| pegRNA | 3523 |
| pegRNA | 3524 |
| pegRNA | 3525 |
| pegRNA | 3526 |
| pegRNA | 3527 |
| pegRNA | 3528 |
| pegRNA | 3529 |
| pegRNA | 3530 |
| pegRNA | 3531 |
| pegRNA | 3532 |
| pegRNA | 3533 |
| pegRNA | 3534 |
| pegRNA | 3535 |
| pegRNA | 3536 |
| pegRNA | 3537 |
| pegRNA | 3538 |
| pegRNA | 3539 |
| pegRNA | 3540 |
| pegRNA | 3541 |
| pegRNA | 3542 |
| pegRNA | 3543 |
| pegRNA | 3544 |
| pegRNA | 3545 |
| pegRNA | 3546 |
| pegRNA | 3547 |
| pegRNA | 3548 |
| pegRNA | 3549 |
| pegRNA | 3550 |
| pegRNA | 3551 |
| pegRNA | 3552 |
| pegRNA | 3553 |
| pegRNA | 3554 |
| pegRNA | 3555 |
| pegRNA | 3556 |
| pegRNA | 3557 |
| pegRNA | 3558 |
| pegRNA | 3559 |
| pegRNA | 3560 |
| pegRNA | 3561 |
| pegRNA | 3562 |
| pegRNA | 3563 |
| pegRNA | 3564 |
| pegRNA | 3565 |
| pegRNA | 3566 |
| pegRNA | 3567 |
| pegRNA | 3568 |
| pegRNA | 3569 |
| pegRNA | 3570 |
| pegRNA | 3571 |
| pegRNA | 3572 |
| pegRNA | 3573 |
| pegRNA | 3574 |
| pegRNA | 3575 |
| pegRNA | 3576 |
| pegRNA | 3577 |
| pegRNA | 3578 |
| pegRNA | 3579 |
| pegRNA | 3580 |
| pegRNA | 3581 |
| pegRNA | 3582 |
| pegRNA | 3583 |
| pegRNA | 3584 |
| pegRNA | 3585 |
| pegRNA | 3586 |
| pegRNA | 3587 |
| pegRNA | 3588 |
| pegRNA | 3589 |
| pegRNA | 3590 |
| pegRNA | 3591 |
| pegRNA | 3592 |
| pegRNA | 3593 |
| pegRNA | 3594 |
| pegRNA | 3595 |
| pegRNA | 3596 |
| pegRNA | 3597 |
| pegRNA | 3598 |
| pegRNA | 3599 |
| pegRNA | 3600 |
| pegRNA | 3601 |
| pegRNA | 3602 |
| pegRNA | 3603 |
| pegRNA | 3604 |
| pegRNA | 3605 |
| pegRNA | 3606 |
| pegRNA | 3607 |
| pegRNA | 3608 |
| pegRNA | 3609 |
| pegRNA | 3610 |
| pegRNA | 3611 |
| pegRNA | 3612 |
| pegRNA | 3613 |
| pegRNA | 3614 |
| pegRNA | 3615 |
| pegRNA | 3616 |
| pegRNA | 3617 |
| pegRNA | 3618 |
| pegRNA | 3619 |
| pegRNA | 3620 |
| pegRNA | 3621 |
| pegRNA | 3622 |
| pegRNA | 3623 |
| pegRNA | 3624 |
| pegRNA | 3625 |
| pegRNA | 3626 |
| pegRNA | 3627 |
| pegRNA | 3628 |
| pegRNA | 3629 |
| pegRNA | 3630 |
| pegRNA | 3631 |
| pegRNA | 3632 |
| pegRNA | 3633 |
| pegRNA | 3634 |
| pegRNA | 3635 |
| pegRNA | 3636 |
| pegRNA | 3637 |
| pegRNA | 3638 |
| pegRNA | 3639 |
| pegRNA | 3640 |
| pegRNA | 3641 |
| pegRNA | 3642 |
| pegRNA | 3643 |
| pegRNA | 3644 |
| pegRNA | 3645 |
| pegRNA | 3646 |
| pegRNA | 3647 |
| pegRNA | 3648 |
| pegRNA | 3649 |
| pegRNA | 3650 |
| pegRNA | 3651 |
| pegRNA | 3652 |
| pegRNA | 3653 |
| pegRNA | 3654 |
| pegRNA | 3655 |
| pegRNA | 3656 |
| pegRNA | 3657 |
| pegRNA | 3658 |
| pegRNA | 3659 |
| pegRNA | 3660 |
| pegRNA | 3661 |
| pegRNA | 3662 |
| pegRNA | 3663 |
| pegRNA | 3664 |
| pegRNA | 3665 |
| pegRNA | 3666 |
| pegRNA | 3667 |
| pegRNA | 3668 |
| pegRNA | 3669 |
| pegRNA | 3670 |
| pegRNA | 3671 |
| pegRNA | 3672 |
| pegRNA | 3673 |
| pegRNA | 3674 |
| pegRNA | 3675 |
| pegRNA | 3676 |
| pegRNA | 3677 |
| pegRNA | 3678 |
| pegRNA | 3679 |
| pegRNA | 3680 |
| pegRNA | 3681 |
| pegRNA | 3682 |
| pegRNA | 3683 |
| pegRNA | 3684 |
| pegRNA | 3685 |
| pegRNA | 3686 |
| pegRNA | 3687 |
| pegRNA | 3688 |
| pegRNA | 3689 |
| pegRNA | 3690 |
| pegRNA | 3691 |
| pegRNA | 3692 |
| pegRNA | 3693 |
| pegRNA | 3694 |
| pegRNA | 3695 |
| pegRNA | 3696 |
| pegRNA | 3697 |
| pegRNA | 3698 |
| pegRNA | 3699 |
| pegRNA | 3700 |
| pegRNA | 3701 |
| pegRNA | 3702 |
| pegRNA | 3703 |
| pegRNA | 3704 |
| pegRNA | 3705 |
| pegRNA | 3706 |
| pegRNA | 3707 |
| pegRNA | 3708 |
| pegRNA | 3709 |
| pegRNA | 3710 |
| pegRNA | 3711 |
| pegRNA | 3712 |
| pegRNA | 3713 |
| pegRNA | 3714 |
| pegRNA | 3715 |
| pegRNA | 3716 |
| pegRNA | 3717 |
| pegRNA | 3718 |
| pegRNA | 3719 |
| pegRNA | 3720 |
| pegRNA | 3721 |
| pegRNA | 3722 |
| pegRNA | 3723 |
| pegRNA | 3724 |
| pegRNA | 3725 |
| pegRNA | 3726 |
| pegRNA | 3727 |
| pegRNA | 3728 |
| pegRNA | 3729 |
| pegRNA | 3730 |
| pegRNA | 3731 |
| pegRNA | 3732 |
| pegRNA | 3733 |
| pegRNA | 3734 |
| pegRNA | 3735 |
| pegRNA | 3736 |
| pegRNA | 3737 |
| pegRNA | 3738 |
| pegRNA | 3739 |
| pegRNA | 3740 |
| pegRNA | 3741 |
| pegRNA | 3742 |
| pegRNA | 3743 |
| pegRNA | 3744 |
| pegRNA | 3745 |
| pegRNA | 3746 |
| pegRNA | 3747 |
| pegRNA | 3748 |
| pegRNA | 3749 |
| pegRNA | 3750 |
| pegRNA | 3751 |
| pegRNA | 3752 |
| pegRNA | 3753 |
| pegRNA | 3754 |
| pegRNA | 3755 |
| pegRNA | 3756 |
| pegRNA | 3757 |
| pegRNA | 3758 |
| pegRNA | 3759 |
| pegRNA | 3760 |
| pegRNA | 3761 |
| pegRNA | 3762 |
| pegRNA | 3763 |
| pegRNA | 3764 |
| pegRNA | 3765 |
| pegRNA | 3766 |
| pegRNA | 3767 |
| pegRNA | 3768 |
| pegRNA | 3769 |
| pegRNA | 3770 |
| pegRNA | 3771 |
| pegRNA | 3772 |
| pegRNA | 3773 |
| pegRNA | 3774 |
| pegRNA | 3775 |
| pegRNA | 3776 |
| pegRNA | 3777 |
| pegRNA | 3778 |
| pegRNA | 3779 |
| pegRNA | 3780 |
| pegRNA | 3781 |
| pegRNA | 3782 |
| pegRNA | 3783 |
| pegRNA | 3784 |
| pegRNA | 3785 |
| pegRNA | 3786 |
| pegRNA | 3787 |
| pegRNA | 3788 |
| pegRNA | 3789 |
| pegRNA | 3790 |
| pegRNA | 3791 |
| pegRNA | 3792 |
| pegRNA | 3793 |
| pegRNA | 3794 |
| pegRNA | 3795 |
| pegRNA | 3796 |
| pegRNA | 3797 |
| pegRNA | 3798 |
| pegRNA | 3799 |
| pegRNA | 3800 |
| pegRNA | 3801 |
| pegRNA | 3802 |
| pegRNA | 3803 |
| pegRNA | 3804 |
| pegRNA | 3805 |
| pegRNA | 3806 |
| pegRNA | 3807 |
| pegRNA | 3808 |
| pegRNA | 3809 |
| pegRNA | 3810 |
| pegRNA | 3811 |
| pegRNA | 3812 |
| pegRNA | 3813 |
| pegRNA | 3814 |
| pegRNA | 3815 |
| pegRNA | 3816 |
| pegRNA | 3817 |
| pegRNA | 3818 |
| pegRNA | 3819 |
| pegRNA | 3820 |
| pegRNA | 3821 |
| pegRNA | 3822 |
| pegRNA | 3823 |
| pegRNA | 3824 |
| pegRNA | 3825 |
| pegRNA | 3826 |
| pegRNA | 3827 |
| pegRNA | 3828 |
| pegRNA | 3829 |
| pegRNA | 3830 |
| pegRNA | 3831 |
| pegRNA | 3832 |
| pegRNA | 3833 |
| pegRNA | 3834 |
| pegRNA | 3835 |
| pegRNA | 3836 |
| pegRNA | 3837 |
| pegRNA | 3838 |
| pegRNA | 3839 |
| pegRNA | 3840 |
| pegRNA | 3841 |
| pegRNA | 3842 |
| pegRNA | 3843 |
| pegRNA | 3844 |
| pegRNA | 3845 |
| pegRNA | 3846 |
| pegRNA | 3847 |
| pegRNA | 3848 |
| pegRNA | 3849 |
| pegRNA | 3850 |
| pegRNA | 3851 |
| pegRNA | 3852 |
| pegRNA | 3853 |
| pegRNA | 3854 |
| pegRNA | 3855 |
| pegRNA | 3856 |
| pegRNA | 3857 |
| pegRNA | 3858 |
| pegRNA | 3859 |
| pegRNA | 3860 |
| pegRNA | 3861 |
| pegRNA | 3862 |
| pegRNA | 3863 |
| pegRNA | 3864 |
| pegRNA | 3865 |
| pegRNA | 3866 |
| pegRNA | 3867 |
| pegRNA | 3868 |
| pegRNA | 3869 |
| pegRNA | 3870 |
| pegRNA | 3871 |
| pegRNA | 3872 |
| pegRNA | 3873 |
| pegRNA | 3874 |
| pegRNA | 3875 |
| pegRNA | 3876 |
| pegRNA | 3877 |
| pegRNA | 3878 |
| pegRNA | 3879 |
| pegRNA | 3880 |
| pegRNA | 3881 |
| pegRNA | 3882 |
| pegRNA | 3883 |
| pegRNA | 3884 |
| pegRNA | 3885 |
| pegRNA | 3886 |
| pegRNA | 3887 |
| pegRNA | 3888 |
| pegRNA | 3889 |
| pegRNA | 3890 |
| pegRNA | 3891 |
| pegRNA | 3892 |
| pegRNA | 3893 |
| pegRNA | 3894 |
| pegRNA | 3895 |
| pegRNA | 3896 |
| pegRNA | 3897 |
| pegRNA | 3898 |
| pegRNA | 3899 |
| pegRNA | 3900 |
| pegRNA | 3901 |
| pegRNA | 3902 |
| pegRNA | 3903 |
| pegRNA | 3904 |
| pegRNA | 3905 |
| pegRNA | 3906 |
| pegRNA | 3907 |
| pegRNA | 3908 |
| pegRNA | 3909 |
| pegRNA | 3910 |
| pegRNA | 3911 |
| pegRNA | 3912 |
| pegRNA | 3913 |
| pegRNA | 3914 |
| pegRNA | 3915 |
| pegRNA | 3916 |
| pegRNA | 3917 |
| pegRNA | 3918 |
| pegRNA | 3919 |
| pegRNA | 3920 |
| pegRNA | 3921 |
| pegRNA | 3922 |
| pegRNA | 3923 |
| pegRNA | 3924 |
| pegRNA | 3925 |
| pegRNA | 3926 |
| pegRNA | 3927 |
| pegRNA | 3928 |
| pegRNA | 3929 |
| pegRNA | 3930 |
| pegRNA | 3931 |
| pegRNA | 3932 |
| pegRNA | 3933 |
| pegRNA | 3934 |
| pegRNA | 3935 |
| pegRNA | 3936 |
| pegRNA | 3937 |
| pegRNA | 3938 |
| pegRNA | 3939 |
| pegRNA | 3940 |
| pegRNA | 3941 |
| pegRNA | 3942 |
| pegRNA | 3943 |
| pegRNA | 3944 |
| pegRNA | 3945 |
| pegRNA | 3946 |
| pegRNA | 3947 |
| pegRNA | 3948 |
| pegRNA | 3949 |
| pegRNA | 3950 |
| pegRNA | 3951 |
| pegRNA | 3952 |
| pegRNA | 3953 |
| pegRNA | 3954 |
| pegRNA | 3955 |
| pegRNA | 3956 |
| pegRNA | 3957 |
| pegRNA | 3958 |
| pegRNA | 3959 |
| pegRNA | 3960 |
| pegRNA | 3961 |
| pegRNA | 3962 |
| pegRNA | 3963 |
| pegRNA | 3964 |
| pegRNA | 3965 |
| pegRNA | 3966 |
| pegRNA | 3967 |
| pegRNA | 3968 |
| pegRNA | 3969 |
| pegRNA | 3970 |
| pegRNA | 3971 |
| pegRNA | 3972 |
| pegRNA | 3973 |
| pegRNA | 3974 |
| pegRNA | 3975 |
| pegRNA | 3976 |
| pegRNA | 3977 |
| pegRNA | 3978 |
| pegRNA | 3979 |
| pegRNA | 3980 |
| pegRNA | 3981 |
| pegRNA | 3982 |
| pegRNA | 3983 |
| pegRNA | 3984 |
| pegRNA | 3985 |
| pegRNA | 3986 |
| pegRNA | 3987 |
| pegRNA | 3988 |
| pegRNA | 3989 |
| pegRNA | 3990 |
| pegRNA | 3991 |
| pegRNA | 3992 |
| pegRNA | 3993 |
| pegRNA | 3994 |
| pegRNA | 3995 |
| pegRNA | 3996 |
| pegRNA | 3997 |
| pegRNA | 3998 |
| pegRNA | 3999 |
| pegRNA | 4000 |
| pegRNA | 4001 |
| pegRNA | 4002 |
| pegRNA | 4003 |
| pegRNA | 4004 |
| pegRNA | 4005 |
| pegRNA | 4006 |
| pegRNA | 4007 |
| pegRNA | 4008 |
| pegRNA | 4009 |
| pegRNA | 4010 |
| pegRNA | 4011 |
| pegRNA | 4012 |
| pegRNA | 4013 |
| pegRNA | 4014 |
| pegRNA | 4015 |
| pegRNA | 4016 |
| pegRNA | 4017 |
| pegRNA | 4018 |
| pegRNA | 4019 |
| pegRNA | 4020 |
| pegRNA | 4021 |
| pegRNA | 4022 |
| pegRNA | 4023 |
| pegRNA | 4024 |
| pegRNA | 4025 |
| pegRNA | 4026 |
| pegRNA | 4027 |
| pegRNA | 4028 |
| pegRNA | 4029 |
| pegRNA | 4030 |
| pegRNA | 4031 |
| pegRNA | 4032 |
| pegRNA | 4033 |
| pegRNA | 4034 |
| pegRNA | 4035 |
| pegRNA | 4036 |
| pegRNA | 4037 |
| pegRNA | 4038 |
| pegRNA | 4039 |
| pegRNA | 4040 |
| pegRNA | 4041 |
| pegRNA | 4042 |
| pegRNA | 4043 |
| pegRNA | 4044 |
| pegRNA | 4045 |
| pegRNA | 4046 |
| pegRNA | 4047 |
| pegRNA | 4048 |
| pegRNA | 4049 |
| pegRNA | 4050 |
| pegRNA | 4051 |
| pegRNA | 4052 |
| pegRNA | 4053 |
| pegRNA | 4054 |
| pegRNA | 4055 |
| pegRNA | 4056 |
| pegRNA | 4057 |
| pegRNA | 4058 |
| pegRNA | 4059 |
| pegRNA | 4060 |
| pegRNA | 4061 |
| pegRNA | 4062 |
| pegRNA | 4063 |
| pegRNA | 4064 |
| pegRNA | 4065 |
| pegRNA | 4066 |
| pegRNA | 4067 |
| pegRNA | 4068 |
| pegRNA | 4069 |
| pegRNA | 4070 |
| pegRNA | 4071 |
| pegRNA | 4072 |
| pegRNA | 4073 |
| pegRNA | 4074 |
| pegRNA | 4075 |
| pegRNA | 4076 |
| pegRNA | 4077 |
| pegRNA | 4078 |
| pegRNA | 4079 |
| pegRNA | 4080 |
| pegRNA | 4081 |
| pegRNA | 4082 |
| pegRNA | 4083 |
| pegRNA | 4084 |
| pegRNA | 4085 |
| pegRNA | 4086 |
| pegRNA | 4087 |
| pegRNA | 4088 |
| pegRNA | 4089 |
| pegRNA | 4090 |
| pegRNA | 4091 |
| pegRNA | 4092 |
| pegRNA | 4093 |
| pegRNA | 4094 |
| pegRNA | 4095 |
| pegRNA | 4096 |
| pegRNA | 4097 |
| pegRNA | 4098 |
| pegRNA | 4099 |
| pegRNA | 4100 |
| pegRNA | 4101 |
| pegRNA | 4102 |
| pegRNA | 4103 |
| pegRNA | 4104 |
| pegRNA | 4105 |
| pegRNA | 4106 |
| pegRNA | 4107 |
| pegRNA | 4108 |
| pegRNA | 4109 |
| pegRNA | 4110 |
| pegRNA | 4111 |
| pegRNA | 4112 |
| pegRNA | 4113 |
| pegRNA | 4114 |
| pegRNA | 4115 |
| pegRNA | 4116 |
| pegRNA | 4117 |
| pegRNA | 4118 |
| pegRNA | 4119 |
| pegRNA | 4120 |
| pegRNA | 4121 |
| pegRNA | 4122 |
| pegRNA | 4123 |
| pegRNA | 4124 |
| pegRNA | 4125 |
| pegRNA | 4126 |
| pegRNA | 4127 |
| pegRNA | 4128 |
| pegRNA | 4129 |
| pegRNA | 4130 |
| pegRNA | 4131 |
| pegRNA | 4132 |
| pegRNA | 4133 |
| pegRNA | 4134 |
| pegRNA | 4135 |
| pegRNA | 4136 |
| pegRNA | 4137 |
| pegRNA | 4138 |
| pegRNA | 4139 |
| pegRNA | 4140 |
| pegRNA | 4141 |
| pegRNA | 4142 |
| pegRNA | 4143 |
| pegRNA | 4144 |
| pegRNA | 4145 |
| pegRNA | 4146 |
| pegRNA | 4147 |
| pegRNA | 4148 |
| pegRNA | 4149 |
| pegRNA | 4150 |
| pegRNA | 4151 |
| pegRNA | 4152 |
| pegRNA | 4153 |
| pegRNA | 4154 |
| pegRNA | 4155 |
| pegRNA | 4156 |
| pegRNA | 4157 |
| pegRNA | 4158 |
| pegRNA | 4159 |
| pegRNA | 4160 |
| pegRNA | 4161 |
| pegRNA | 4162 |
| pegRNA | 4163 |
| pegRNA | 4164 |
| pegRNA | 4165 |
| pegRNA | 4166 |
| pegRNA | 4167 |
| pegRNA | 4168 |
| pegRNA | 4169 |
| pegRNA | 4170 |
| pegRNA | 4171 |
| pegRNA | 4172 |
| pegRNA | 4173 |
| pegRNA | 4174 |
| pegRNA | 4175 |
| pegRNA | 4176 |
| pegRNA | 4177 |
| pegRNA | 4178 |
| pegRNA | 4179 |
| pegRNA | 4180 |
| pegRNA | 4181 |
| pegRNA | 4182 |
| pegRNA | 4183 |
| pegRNA | 4184 |
| pegRNA | 4185 |
| pegRNA | 4186 |
| pegRNA | 4187 |
| pegRNA | 4188 |
| pegRNA | 4189 |
| pegRNA | 4190 |
| pegRNA | 4191 |
| pegRNA | 4192 |
| pegRNA | 4193 |
| pegRNA | 4194 |
| pegRNA | 4195 |
| pegRNA | 4196 |
| pegRNA | 4197 |
| pegRNA | 4198 |
| pegRNA | 4199 |
| pegRNA | 4200 |
| pegRNA | 4201 |
| pegRNA | 4202 |
| pegRNA | 4203 |
| pegRNA | 4204 |
| pegRNA | 4205 |
| pegRNA | 4206 |
| pegRNA | 4207 |
| pegRNA | 4208 |
| pegRNA | 4209 |
| pegRNA | 4210 |
| pegRNA | 4211 |
| pegRNA | 4212 |
| pegRNA | 4213 |
| pegRNA | 4214 |
| pegRNA | 4215 |
| pegRNA | 4216 |
| pegRNA | 4217 |
| pegRNA | 4218 |
| pegRNA | 4219 |
| pegRNA | 4220 |
| pegRNA | 4221 |
| pegRNA | 4222 |
| pegRNA | 4223 |
| pegRNA | 4224 |
| pegRNA | 4225 |
| pegRNA | 4226 |
| pegRNA | 4227 |
| pegRNA | 4228 |
| pegRNA | 4229 |
| pegRNA | 4230 |
| pegRNA | 4231 |
| pegRNA | 4232 |
| pegRNA | 4233 |
| pegRNA | 4234 |
| pegRNA | 4235 |
| pegRNA | 4236 |
| pegRNA | 4237 |
| pegRNA | 4238 |
| pegRNA | 4239 |
| pegRNA | 4240 |
| pegRNA | 4241 |
| pegRNA | 4242 |
| pegRNA | 4243 |
| pegRNA | 4244 |
| pegRNA | 4245 |
| pegRNA | 4246 |
| pegRNA | 4247 |
| pegRNA | 4248 |
| pegRNA | 4249 |
| pegRNA | 4250 |
| pegRNA | 4251 |
| pegRNA | 4252 |
| pegRNA | 4253 |
| pegRNA | 4254 |
| pegRNA | 4255 |
| pegRNA | 4256 |
| pegRNA | 4257 |
| pegRNA | 4258 |
| pegRNA | 4259 |
| pegRNA | 4260 |
| pegRNA | 4261 |
| pegRNA | 4262 |
| pegRNA | 4263 |
| pegRNA | 4264 |
| pegRNA | 4265 |
| pegRNA | 4266 |
| pegRNA | 4267 |
| pegRNA | 4268 |
| pegRNA | 4269 |
| pegRNA | 4270 |
| pegRNA | 4271 |
| pegRNA | 4272 |
| pegRNA | 4273 |
| pegRNA | 4274 |
| pegRNA | 4275 |
| pegRNA | 4276 |
| pegRNA | 4277 |
| pegRNA | 4278 |
| pegRNA | 4279 |
| pegRNA | 4280 |
| pegRNA | 4281 |
| pegRNA | 4282 |
| pegRNA | 4283 |
| pegRNA | 4284 |
| pegRNA | 4285 |
| pegRNA | 4286 |
| pegRNA | 4287 |
| pegRNA | 4288 |
| pegRNA | 4289 |
| pegRNA | 4290 |
| pegRNA | 4291 |
| pegRNA | 4292 |
| pegRNA | 4293 |
| pegRNA | 4294 |
| pegRNA | 4295 |
| pegRNA | 4296 |
| pegRNA | 4297 |
| pegRNA | 4298 |
| pegRNA | 4299 |
| pegRNA | 4300 |
| pegRNA | 4301 |
| pegRNA | 4302 |
| pegRNA | 4303 |
| pegRNA | 4304 |
| pegRNA | 4305 |
| pegRNA | 4306 |
| pegRNA | 4307 |
| pegRNA | 4308 |
| pegRNA | 4309 |
| pegRNA | 4310 |
| pegRNA | 4311 |
| pegRNA | 4312 |
| pegRNA | 4313 |
| pegRNA | 4314 |
| pegRNA | 4315 |
| pegRNA | 4316 |
| pegRNA | 4317 |
| pegRNA | 4318 |
| pegRNA | 4319 |
| pegRNA | 4320 |
| pegRNA | 4321 |
| pegRNA | 4322 |
| pegRNA | 4323 |
| pegRNA | 4324 |
| pegRNA | 4325 |
| pegRNA | 4326 |
| pegRNA | 4327 |
| pegRNA | 4328 |
| pegRNA | 4329 |
| pegRNA | 4330 |
| pegRNA | 4331 |
| pegRNA | 4332 |
| pegRNA | 4333 |
| pegRNA | 4334 |
| pegRNA | 4335 |
| pegRNA | 4336 |
| pegRNA | 4337 |
| pegRNA | 4338 |
| pegRNA | 4339 |
| pegRNA | 4340 |
| pegRNA | 4341 |
| pegRNA | 4342 |
| pegRNA | 4343 |
| pegRNA | 4344 |
| pegRNA | 4345 |
| pegRNA | 4346 |
| pegRNA | 4347 |
| pegRNA | 4348 |
| pegRNA | 4349 |
| pegRNA | 4350 |
| pegRNA | 4351 |
| pegRNA | 4352 |
| pegRNA | 4353 |
| pegRNA | 4354 |
| pegRNA | 4355 |
| pegRNA | 4356 |
| pegRNA | 4357 |
| pegRNA | 4358 |
| pegRNA | 4359 |
| pegRNA | 4360 |
| pegRNA | 4361 |
| pegRNA | 4362 |
| pegRNA | 4363 |
| pegRNA | 4364 |
| pegRNA | 4365 |
| pegRNA | 4366 |
| pegRNA | 4367 |
| pegRNA | 4368 |
| pegRNA | 4369 |
| pegRNA | 4370 |
| pegRNA | 4371 |
| pegRNA | 4372 |
| pegRNA | 4373 |
| pegRNA | 4374 |
| pegRNA | 4375 |
| pegRNA | 4376 |
| pegRNA | 4377 |
| pegRNA | 4378 |
| pegRNA | 4379 |
| pegRNA | 4380 |
| pegRNA | 4381 |
| pegRNA | 4382 |
| pegRNA | 4383 |
| pegRNA | 4384 |
| pegRNA | 4385 |
| pegRNA | 4386 |
| pegRNA | 4387 |
| pegRNA | 4388 |
| pegRNA | 4389 |
| pegRNA | 4390 |
| pegRNA | 4391 |
| pegRNA | 4392 |
| pegRNA | 4393 |
| pegRNA | 4394 |
| pegRNA | 4395 |
| pegRNA | 4396 |
| pegRNA | 4397 |
| pegRNA | 4398 |
| pegRNA | 4399 |
| pegRNA | 4400 |
| pegRNA | 4401 |
| pegRNA | 4402 |
| pegRNA | 4403 |
| pegRNA | 4404 |
| pegRNA | 4405 |
| pegRNA | 4406 |
| pegRNA | 4407 |
| pegRNA | 4408 |
| pegRNA | 4409 |
| ngRNA | 2268 |
| ngRNA | 2257 |
| ngRNA | 4410 |
| ngRNA | 4411 |
| ngRNA | 4412 |
| ngRNA | 2275 |
| ngRNA | 2267 |
| ngRNA | 2266 |
| ngRNA | 4413 |
| ngRNA | 2272 |
| ngRNA | 2271 |
| ngRNA | 4414 |
| ngRNA | 2270 |
| ngRNA | 2263 |
| ngRNA | 2265 |
| ngRNA | 2261 |
| ngRNA | 2264 |
| ngRNA | 4415 |
| ngRNA | 2258 |
| ngRNA | 2276 |
| ngRNA | 2262 |
| ngRNA | 2273 |
| ngRNA | 2274 |
| ngRNA | 2260 |
| ngRNA | 4416 |
| ngRNA | 2269 |
| ngRNA | 4417 |
| ngRNA | 2277 |
| ngRNA | 4418 |
| ngRNA | 2259 |
| ngRNA | 4419 |
| ngRNA | 2280 |
| ngRNA | 4420 |
| ngRNA | 4421 |
| ngRNA | 2286 |
| ngRNA | 4422 |
| ngRNA | 2288 |
| ngRNA | 2289 |
| ngRNA | 2279 |
| ngRNA | 2281 |
| ngRNA | 2282 |
| ngRNA | 2285 |
| ngRNA | 2283 |
| ngRNA | 2287 |
| ngRNA | 2278 |
| ngRNA | 2284 |
| ngRNA | 4423 |
| ngRNA | 2291 |
| ngRNA | 4424 |
| ngRNA | 2290 |
| ngRNA | 2292 |

TABLE 16

| Sequence region | SEQ ID |
|---|---|
| pegRNA Spacer | 4425 |
| PBS | 4426 |
| PBS | 4427 |
| PBS | 4428 |
| PBS | 2297 |
| PBS | 4429 |
| PBS | 4430 |
| PBS | 4431 |
| PBS | 4432 |
| PBS | 4433 |
| PBS | 4434 |
| PBS | 4435 |
| PBS | 4436 |
| RTT | 4437 |
| RTT | 4438 |
| RTT | 4439 |
| RTT | 4440 |
| RTT | 4441 |
| RTT | 4442 |
| RTT | 4443 |
| RTT | 4444 |
| RTT | 4445 |
| RTT | 4446 |
| RTT | 4447 |
| RTT | 4448 |
| RTT | 4449 |
| RTT | 4450 |
| RTT | 4451 |
| RTT | 4452 |
| RTT | 4453 |
| RTT | 4454 |
| RTT | 4455 |
| RTT | 4456 |
| RTT | 4457 |
| RTT | 4458 |
| RTT | 4459 |
| RTT | 4460 |
| RTT | 4461 |
| RTT | 4462 |
| RTT | 4463 |
| RTT | 4464 |
| RTT | 4465 |
| RTT | 4466 |
| RTT | 4467 |
| RTT | 4468 |
| RTT | 4469 |
| RTT | 4470 |
| RTT | 4471 |
| RTT | 4472 |
| RTT | 4473 |
| RTT | 4474 |
| RTT | 4475 |
| RTT | 4476 |
| RTT | 4477 |
| RTT | 4478 |
| RTT | 4479 |
| RTT | 4480 |
| RTT | 4481 |
| RTT | 4482 |
| RTT | 4483 |
| RTT | 4484 |
| RTT | 4485 |
| RTT | 4486 |
| RTT | 4487 |
| RTT | 4488 |
| RTT | 4489 |
| RTT | 4490 |
| RTT | 4491 |
| RTT | 4492 |
| ngRNA Spacer | 2083 |
| ngRNA Spacer | 2080 |
| ngRNA Spacer | 2091 |
| ngRNA Spacer | 2442 |
| ngRNA Spacer | 2066 |
| ngRNA Spacer | 2067 |
| ngRNA Spacer | 2075 |
| ngRNA Spacer | 2057 |
| ngRNA Spacer | 2050 |
| ngRNA Spacer | 2058 |
| ngRNA Spacer | 2072 |
| ngRNA Spacer | 2076 |
| ngRNA Spacer | 2079 |
| ngRNA Spacer | 2078 |
| ngRNA Spacer | 2051 |
| ngRNA Spacer | 2046 |
| ngRNA Spacer | 2065 |
| ngRNA Spacer | 2085 |
| ngRNA Spacer | 2049 |
| ngRNA Spacer | 2047 |
| ngRNA Spacer | 2061 |
| ngRNA Spacer | 2064 |
| ngRNA Spacer | 2068 |
| ngRNA Spacer | 2082 |
| ngRNA Spacer | 2054 |
| ngRNA Spacer | 2062 |
| ngRNA Spacer | 2056 |
| ngRNA Spacer | 2094 |
| ngRNA Spacer | 2045 |
| ngRNA Spacer | 2090 |
| ngRNA Spacer | 2086 |
| ngRNA Spacer | 2074 |
| ngRNA Spacer | 2095 |
| ngRNA Spacer | 2084 |
| ngRNA Spacer | 65 |
| ngRNA Spacer | 2059 |
| ngRNA Spacer | 71 |
| ngRNA Spacer | 66 |
| ngRNA Spacer | 62 |
| ngRNA Spacer | 63 |
| ngRNA Spacer | 64 |
| ngRNA Spacer | 69 |
| ngRNA Spacer | 2088 |
| ngRNA Spacer | 2070 |
| ngRNA Spacer | 60 |
| ngRNA Spacer | 61 |
| ngRNA Spacer | 2089 |
| ngRNA Spacer | 2063 |
| ngRNA Spacer | 2093 |
| ngRNA Spacer | 2073 |
| ngRNA Spacer | 2081 |
| ngRNA Spacer | 2071 |
| ngRNA Spacer | 2055 |
| ngRNA Spacer | 2087 |
| ngRNA Spacer | 2069 |
| ngRNA Spacer | 2048 |
| ngRNA Spacer | 2437 |
| ngRNA Spacer | 41 |
| ngRNA Spacer | 2077 |
| ngRNA Spacer | 2439 |
| ngRNA Spacer | 2092 |
| ngRNA Spacer | 2060 |
| ngRNA Spacer | 2052 |
| ngRNA Spacer | 2443 |
| ngRNA Spacer | 2053 |
| ngRNA Spacer | 2096 |
| ngRNA Spacer | 2444 |
| pegRNA | 4493 |
| pegRNA | 4494 |
| pegRNA | 4495 |
| pegRNA | 4496 |
| pegRNA | 4497 |
| pegRNA | 4498 |
| pegRNA | 4499 |
| pegRNA | 4500 |
| pegRNA | 4501 |
| pegRNA | 4502 |
| pegRNA | 4503 |
| pegRNA | 4504 |
| pegRNA | 4505 |
| pegRNA | 4504 |
| pegRNA | 4506 |
| pegRNA | 4507 |
| pegRNA | 4508 |
| pegRNA | 4509 |
| pegRNA | 4510 |
| pegRNA | 4511 |
| pegRNA | 4512 |
| pegRNA | 4513 |
| pegRNA | 4514 |
| pegRNA | 4515 |
| pegRNA | 4516 |
| pegRNA | 4517 |
| pegRNA | 4518 |
| pegRNA | 4519 |
| pegRNA | 4520 |
| pegRNA | 4521 |
| pegRNA | 4522 |
| pegRNA | 4523 |
| pegRNA | 4524 |
| pegRNA | 4525 |
| pegRNA | 4526 |
| pegRNA | 4527 |
| pegRNA | 4528 |
| pegRNA | 4529 |
| pegRNA | 4522 |
| pegRNA | 4530 |
| pegRNA | 4531 |
| pegRNA | 4532 |
| pegRNA | 4533 |
| pegRNA | 4534 |
| pegRNA | 4535 |
| pegRNA | 4536 |
| pegRNA | 4537 |
| pegRNA | 4538 |
| pegRNA | 4539 |
| pegRNA | 4540 |
| pegRNA | 4541 |
| pegRNA | 4542 |
| pegRNA | 4543 |
| pegRNA | 4544 |
| pegRNA | 4545 |
| pegRNA | 4546 |
| pegRNA | 4547 |
| pegRNA | 4548 |
| pegRNA | 4549 |
| pegRNA | 4550 |
| pegRNA | 4551 |
| pegRNA | 4552 |
| pegRNA | 4553 |
| pegRNA | 4554 |
| pegRNA | 4555 |
| pegRNA | 4556 |
| pegRNA | 4557 |
| pegRNA | 4558 |
| pegRNA | 4559 |
| pegRNA | 4560 |
| pegRNA | 4559 |
| pegRNA | 4561 |
| pegRNA | 4562 |
| pegRNA | 4563 |
| pegRNA | 4564 |
| pegRNA | 4565 |
| pegRNA | 4566 |
| pegRNA | 4567 |
| pegRNA | 4568 |
| pegRNA | 4569 |
| pegRNA | 4570 |
| pegRNA | 4571 |
| pegRNA | 4572 |
| pegRNA | 4573 |
| pegRNA | 4574 |
| pegRNA | 4575 |
| pegRNA | 4576 |
| pegRNA | 4577 |
| pegRNA | 4578 |
| pegRNA | 4579 |
| pegRNA | 4580 |
| pegRNA | 4581 |
| pegRNA | 4582 |
| pegRNA | 4583 |
| pegRNA | 4584 |
| pegRNA | 4585 |
| pegRNA | 4586 |
| pegRNA | 4587 |
| pegRNA | 4588 |
| pegRNA | 4589 |
| pegRNA | 4590 |
| pegRNA | 4591 |
| pegRNA | 4592 |
| pegRNA | 4593 |
| pegRNA | 4594 |
| pegRNA | 4595 |
| pegRNA | 4588 |
| pegRNA | 4596 |
| pegRNA | 4591 |
| pegRNA | 4597 |
| pegRNA | 4598 |
| pegRNA | 4599 |
| pegRNA | 4600 |
| pegRNA | 4601 |
| pegRNA | 4602 |
| pegRNA | 4603 |
| pegRNA | 4604 |
| pegRNA | 4605 |
| pegRNA | 4606 |
| pegRNA | 4607 |
| pegRNA | 4608 |
| pegRNA | 4609 |
| pegRNA | 4610 |
| pegRNA | 4611 |
| pegRNA | 4612 |
| pegRNA | 4613 |
| pegRNA | 4614 |
| pegRNA | 4615 |
| pegRNA | 4616 |
| pegRNA | 4617 |
| pegRNA | 4618 |
| pegRNA | 4619 |
| pegRNA | 4620 |
| pegRNA | 4621 |
| pegRNA | 4622 |
| pegRNA | 4623 |
| pegRNA | 4624 |
| pegRNA | 4625 |
| pegRNA | 4626 |
| pegRNA | 4627 |
| pegRNA | 4628 |
| pegRNA | 4629 |
| pegRNA | 4630 |
| pegRNA | 4631 |
| pegRNA | 4632 |
| pegRNA | 4633 |
| pegRNA | 4634 |
| pegRNA | 4635 |
| pegRNA | 4636 |
| pegRNA | 4637 |
| pegRNA | 4638 |
| pegRNA | 4639 |
| pegRNA | 4640 |
| pegRNA | 4641 |
| pegRNA | 4642 |
| pegRNA | 4643 |
| pegRNA | 4644 |
| pegRNA | 4645 |
| pegRNA | 4646 |
| pegRNA | 4647 |
| pegRNA | 4648 |
| pegRNA | 4649 |
| pegRNA | 4650 |
| pegRNA | 4651 |
| pegRNA | 4652 |
| pegRNA | 4653 |
| pegRNA | 4654 |
| pegRNA | 4655 |
| pegRNA | 4656 |
| pegRNA | 4657 |
| pegRNA | 4658 |
| pegRNA | 4659 |
| pegRNA | 4660 |
| pegRNA | 4661 |
| pegRNA | 4662 |
| pegRNA | 4663 |
| pegRNA | 4664 |
| pegRNA | 4665 |
| pegRNA | 4666 |
| pegRNA | 4667 |
| pegRNA | 4668 |
| pegRNA | 4669 |
| pegRNA | 4670 |
| pegRNA | 4671 |
| pegRNA | 4672 |
| pegRNA | 4673 |
| pegRNA | 4674 |
| pegRNA | 4675 |
| pegRNA | 4676 |
| pegRNA | 4677 |
| pegRNA | 4678 |
| pegRNA | 4679 |
| pegRNA | 4680 |
| pegRNA | 4681 |
| pegRNA | 4682 |
| pegRNA | 4683 |
| pegRNA | 4684 |
| pegRNA | 4685 |
| pegRNA | 4686 |
| pegRNA | 4687 |
| pegRNA | 4688 |
| pegRNA | 4689 |
| pegRNA | 4690 |
| pegRNA | 4691 |
| pegRNA | 4692 |
| pegRNA | 4693 |
| pegRNA | 4694 |
| pegRNA | 4695 |
| pegRNA | 4696 |
| pegRNA | 4697 |
| pegRNA | 4698 |
| pegRNA | 4699 |
| pegRNA | 4700 |
| pegRNA | 4701 |
| pegRNA | 4702 |
| pegRNA | 4703 |
| pegRNA | 4697 |
| pegRNA | 4704 |
| pegRNA | 4700 |
| pegRNA | 4705 |
| pegRNA | 4706 |
| pegRNA | 4707 |
| pegRNA | 4708 |
| pegRNA | 4709 |
| pegRNA | 4710 |
| pegRNA | 4711 |
| pegRNA | 4712 |
| pegRNA | 4713 |
| pegRNA | 4714 |
| pegRNA | 4715 |
| pegRNA | 4716 |
| pegRNA | 4717 |
| pegRNA | 4718 |
| pegRNA | 4719 |
| pegRNA | 4720 |
| pegRNA | 4721 |
| pegRNA | 4722 |
| pegRNA | 4723 |
| pegRNA | 4724 |
| pegRNA | 4725 |
| pegRNA | 4726 |
| pegRNA | 4727 |
| pegRNA | 4728 |
| pegRNA | 4729 |
| pegRNA | 4730 |
| pegRNA | 4731 |
| pegRNA | 4732 |
| pegRNA | 4733 |
| pegRNA | 4734 |
| pegRNA | 4735 |
| pegRNA | 4736 |
| pegRNA | 4737 |
| pegRNA | 4738 |
| pegRNA | 4739 |
| pegRNA | 4740 |
| pegRNA | 4741 |
| pegRNA | 4742 |
| pegRNA | 4743 |
| pegRNA | 4744 |
| pegRNA | 4745 |
| pegRNA | 4746 |
| pegRNA | 4747 |
| pegRNA | 4748 |
| pegRNA | 4749 |
| pegRNA | 4750 |
| pegRNA | 4751 |
| pegRNA | 4752 |
| pegRNA | 4753 |
| pegRNA | 4754 |
| pegRNA | 4755 |
| pegRNA | 4756 |
| pegRNA | 4757 |
| pegRNA | 4758 |
| pegRNA | 4759 |
| pegRNA | 4760 |
| pegRNA | 4761 |
| pegRNA | 4762 |
| pegRNA | 4763 |
| pegRNA | 4764 |
| pegRNA | 4765 |
| pegRNA | 4766 |
| pegRNA | 4767 |
| pegRNA | 4768 |
| pegRNA | 4769 |
| pegRNA | 4770 |
| pegRNA | 4771 |
| pegRNA | 4772 |
| pegRNA | 4773 |
| pegRNA | 4774 |
| pegRNA | 4775 |
| pegRNA | 4776 |
| pegRNA | 4777 |
| pegRNA | 4778 |
| pegRNA | 4779 |
| pegRNA | 4780 |
| pegRNA | 4781 |
| pegRNA | 4782 |
| pegRNA | 4783 |
| pegRNA | 4784 |
| pegRNA | 4785 |
| pegRNA | 4786 |
| pegRNA | 4763 |
| pegRNA | 4787 |
| pegRNA | 4788 |
| pegRNA | 4789 |
| pegRNA | 4790 |
| pegRNA | 4791 |
| pegRNA | 4792 |
| pegRNA | 4793 |
| pegRNA | 4794 |
| pegRNA | 4795 |
| pegRNA | 4796 |
| pegRNA | 4797 |
| pegRNA | 4798 |
| pegRNA | 4799 |
| pegRNA | 4800 |
| pegRNA | 4801 |
| pegRNA | 4802 |
| pegRNA | 4803 |
| pegRNA | 4804 |
| pegRNA | 4805 |
| pegRNA | 4806 |
| pegRNA | 4807 |
| pegRNA | 4808 |
| pegRNA | 4809 |
| pegRNA | 4810 |
| pegRNA | 4811 |
| pegRNA | 4812 |
| pegRNA | 4813 |
| pegRNA | 4814 |
| pegRNA | 4815 |
| pegRNA | 4816 |
| pegRNA | 4817 |
| pegRNA | 4818 |
| pegRNA | 4819 |
| pegRNA | 4820 |
| pegRNA | 4821 |
| pegRNA | 4822 |
| pegRNA | 4823 |
| pegRNA | 4824 |
| pegRNA | 4825 |
| pegRNA | 4826 |
| pegRNA | 4827 |
| pegRNA | 4828 |
| pegRNA | 4829 |
| pegRNA | 4830 |
| pegRNA | 4831 |
| pegRNA | 4832 |
| pegRNA | 4833 |
| pegRNA | 4834 |
| pegRNA | 4835 |
| pegRNA | 4836 |
| pegRNA | 4837 |
| pegRNA | 4838 |
| pegRNA | 4839 |
| pegRNA | 4840 |
| pegRNA | 4841 |
| pegRNA | 4826 |
| pegRNA | 4842 |
| pegRNA | 4843 |
| pegRNA | 4844 |
| pegRNA | 4845 |
| pegRNA | 4846 |
| pegRNA | 4847 |
| pegRNA | 4848 |
| pegRNA | 4849 |
| pegRNA | 4850 |
| pegRNA | 4851 |
| pegRNA | 4852 |
| pegRNA | 4853 |
| pegRNA | 4854 |
| pegRNA | 4855 |
| pegRNA | 4856 |
| pegRNA | 4857 |
| pegRNA | 4858 |
| pegRNA | 4859 |
| pegRNA | 4860 |
| pegRNA | 4861 |
| pegRNA | 4862 |
| pegRNA | 4863 |
| pegRNA | 4864 |
| pegRNA | 4865 |
| pegRNA | 4866 |
| pegRNA | 4867 |
| pegRNA | 4868 |
| pegRNA | 4869 |
| pegRNA | 4870 |
| pegRNA | 4871 |
| pegRNA | 4872 |
| pegRNA | 4873 |
| pegRNA | 4874 |
| pegRNA | 4875 |
| pegRNA | 4873 |
| pegRNA | 4876 |
| pegRNA | 4877 |
| pegRNA | 4878 |
| pegRNA | 4879 |
| pegRNA | 4871 |
| pegRNA | 4880 |
| pegRNA | 4881 |
| pegRNA | 4882 |
| pegRNA | 4883 |
| pegRNA | 4884 |
| pegRNA | 4885 |
| pegRNA | 4886 |
| pegRNA | 4887 |
| pegRNA | 4888 |
| pegRNA | 4889 |
| pegRNA | 4890 |
| pegRNA | 4891 |
| pegRNA | 4892 |
| pegRNA | 4893 |
| pegRNA | 4894 |
| pegRNA | 4895 |
| pegRNA | 4896 |
| pegRNA | 4897 |
| pegRNA | 4898 |
| pegRNA | 4899 |
| pegRNA | 4900 |
| pegRNA | 4901 |
| pegRNA | 4902 |
| pegRNA | 4903 |
| pegRNA | 4904 |
| pegRNA | 4905 |
| pegRNA | 4906 |
| pegRNA | 4907 |
| pegRNA | 4908 |
| pegRNA | 4907 |
| pegRNA | 4909 |
| pegRNA | 4910 |
| pegRNA | 4911 |
| pegRNA | 4912 |
| pegRNA | 4913 |
| pegRNA | 4914 |
| pegRNA | 4915 |
| pegRNA | 4916 |
| pegRNA | 4917 |
| pegRNA | 4918 |
| pegRNA | 4919 |
| pegRNA | 4920 |
| pegRNA | 4921 |
| pegRNA | 4922 |
| pegRNA | 4923 |
| pegRNA | 4924 |
| pegRNA | 4925 |
| pegRNA | 4926 |
| pegRNA | 4927 |
| pegRNA | 4928 |
| pegRNA | 4929 |
| pegRNA | 4930 |
| pegRNA | 4931 |
| pegRNA | 4932 |
| pegRNA | 4933 |
| pegRNA | 4934 |
| pegRNA | 4935 |
| pegRNA | 4936 |
| pegRNA | 4937 |
| pegRNA | 4938 |
| pegRNA | 4939 |
| pegRNA | 4940 |
| pegRNA | 4941 |
| pegRNA | 4942 |
| pegRNA | 4943 |
| pegRNA | 4944 |
| pegRNA | 4945 |
| pegRNA | 4946 |
| pegRNA | 4947 |
| pegRNA | 4948 |
| pegRNA | 4949 |
| pegRNA | 4950 |
| pegRNA | 4951 |
| pegRNA | 4952 |
| pegRNA | 4949 |
| pegRNA | 4953 |
| pegRNA | 4951 |
| pegRNA | 4954 |
| pegRNA | 4955 |
| pegRNA | 4956 |
| pegRNA | 4957 |
| pegRNA | 4958 |
| pegRNA | 4959 |
| pegRNA | 4960 |
| pegRNA | 4961 |
| pegRNA | 4962 |
| pegRNA | 4963 |
| pegRNA | 4964 |
| pegRNA | 4965 |
| pegRNA | 4966 |
| pegRNA | 4967 |
| pegRNA | 4968 |
| pegRNA | 4969 |
| pegRNA | 4970 |
| pegRNA | 4971 |
| pegRNA | 4972 |
| pegRNA | 4973 |
| pegRNA | 4974 |
| pegRNA | 4970 |
| pegRNA | 4975 |
| pegRNA | 4976 |
| pegRNA | 4977 |
| pegRNA | 4978 |
| pegRNA | 4979 |
| pegRNA | 4971 |
| pegRNA | 4980 |
| pegRNA | 4981 |
| pegRNA | 4982 |
| pegRNA | 4983 |
| pegRNA | 4984 |
| pegRNA | 4985 |
| pegRNA | 4986 |
| pegRNA | 4987 |
| pegRNA | 4988 |
| pegRNA | 4989 |
| pegRNA | 4990 |
| pegRNA | 4991 |
| pegRNA | 4992 |
| pegRNA | 4993 |
| pegRNA | 4994 |
| pegRNA | 4995 |
| pegRNA | 4996 |
| pegRNA | 4997 |
| pegRNA | 4998 |
| pegRNA | 4999 |
| pegRNA | 5000 |
| pegRNA | 5001 |
| pegRNA | 5002 |
| pegRNA | 5003 |
| pegRNA | 5004 |
| pegRNA | 5005 |
| pegRNA | 5006 |
| pegRNA | 5007 |
| pegRNA | 5008 |
| pegRNA | 5009 |
| pegRNA | 5010 |
| pegRNA | 5004 |
| pegRNA | 5005 |
| pegRNA | 5011 |
| pegRNA | 5012 |
| pegRNA | 5013 |
| pegRNA | 5014 |
| pegRNA | 5015 |
| pegRNA | 5016 |
| pegRNA | 5017 |
| pegRNA | 5018 |
| pegRNA | 5019 |
| pegRNA | 5020 |
| pegRNA | 5021 |
| pegRNA | 5022 |
| pegRNA | 5023 |
| pegRNA | 5024 |
| pegRNA | 5025 |
| pegRNA | 5026 |
| pegRNA | 5027 |
| pegRNA | 5028 |
| pegRNA | 5029 |
| pegRNA | 5030 |
| pegRNA | 5031 |
| pegRNA | 5032 |
| pegRNA | 5033 |
| pegRNA | 5034 |
| pegRNA | 5033 |
| pegRNA | 5035 |
| pegRNA | 5036 |
| pegRNA | 5037 |
| pegRNA | 5038 |
| pegRNA | 5039 |
| pegRNA | 5039 |
| pegRNA | 5040 |
| pegRNA | 5041 |
| pegRNA | 5042 |
| pegRNA | 5043 |
| pegRNA | 5044 |
| pegRNA | 5045 |
| pegRNA | 5046 |
| pegRNA | 5047 |
| pegRNA | 5048 |
| pegRNA | 5049 |
| pegRNA | 5050 |
| pegRNA | 5051 |
| pegRNA | 5052 |
| pegRNA | 5053 |
| pegRNA | 5054 |
| pegRNA | 5055 |
| pegRNA | 5056 |
| pegRNA | 5057 |
| pegRNA | 5058 |
| pegRNA | 5059 |
| pegRNA | 5060 |
| pegRNA | 5058 |
| pegRNA | 5061 |
| pegRNA | 5062 |
| pegRNA | 5063 |
| pegRNA | 5064 |
| pegRNA | 5065 |
| pegRNA | 5066 |
| pegRNA | 5067 |
| pegRNA | 5063 |
| pegRNA | 5068 |
| pegRNA | 5069 |
| pegRNA | 5070 |
| pegRNA | 5071 |
| pegRNA | 5072 |
| pegRNA | 5073 |
| pegRNA | 5074 |
| pegRNA | 5075 |
| pegRNA | 5076 |
| pegRNA | 5077 |
| pegRNA | 5078 |
| pegRNA | 5079 |
| pegRNA | 5080 |
| pegRNA | 5081 |
| pegRNA | 5082 |
| pegRNA | 5083 |
| pegRNA | 5084 |
| pegRNA | 5085 |
| pegRNA | 5086 |
| pegRNA | 5084 |
| pegRNA | 5087 |
| pegRNA | 5088 |
| pegRNA | 5089 |
| pegRNA | 5090 |
| pegRNA | 5091 |
| pegRNA | 5092 |
| pegRNA | 5093 |
| pegRNA | 5094 |
| pegRNA | 5095 |
| pegRNA | 5096 |
| pegRNA | 5097 |
| pegRNA | 5098 |
| pegRNA | 5099 |
| pegRNA | 5100 |
| pegRNA | 5101 |
| pegRNA | 5102 |
| pegRNA | 5103 |
| pegRNA | 5104 |
| pegRNA | 5105 |
| pegRNA | 5106 |
| pegRNA | 5107 |
| pegRNA | 5108 |
| pegRNA | 5109 |
| pegRNA | 5110 |
| pegRNA | 5111 |
| pegRNA | 5112 |
| pegRNA | 5113 |
| pegRNA | 5114 |
| pegRNA | 5115 |
| pegRNA | 5116 |
| pegRNA | 5117 |
| pegRNA | 5115 |
| pegRNA | 5118 |
| pegRNA | 5119 |
| pegRNA | 5120 |
| pegRNA | 5121 |
| pegRNA | 5122 |
| pegRNA | 5123 |
| pegRNA | 5124 |
| pegRNA | 5125 |
| pegRNA | 5126 |
| pegRNA | 5127 |
| pegRNA | 5128 |
| pegRNA | 5129 |
| pegRNA | 5130 |
| pegRNA | 5131 |
| pegRNA | 5132 |
| pegRNA | 5133 |
| pegRNA | 5134 |
| pegRNA | 5135 |
| pegRNA | 5136 |
| pegRNA | 5137 |
| pegRNA | 5138 |
| pegRNA | 5139 |
| pegRNA | 5140 |
| pegRNA | 5141 |
| pegRNA | 5142 |
| pegRNA | 5143 |
| pegRNA | 5144 |
| pegRNA | 5145 |
| pegRNA | 5146 |
| pegRNA | 5147 |
| pegRNA | 5148 |
| pegRNA | 5149 |
| pegRNA | 5150 |
| pegRNA | 5151 |
| pegRNA | 5152 |
| pegRNA | 5153 |
| pegRNA | 5154 |
| pegRNA | 5155 |
| pegRNA | 5156 |
| pegRNA | 5157 |
| pegRNA | 5158 |
| pegRNA | 5159 |
| pegRNA | 5160 |
| pegRNA | 5161 |
| pegRNA | 5162 |
| pegRNA | 5163 |
| pegRNA | 5164 |
| pegRNA | 5165 |
| pegRNA | 5166 |
| pegRNA | 5167 |
| pegRNA | 5168 |
| pegRNA | 5169 |
| pegRNA | 5170 |
| pegRNA | 5171 |
| pegRNA | 5172 |
| pegRNA | 5173 |
| pegRNA | 5174 |
| pegRNA | 5175 |
| pegRNA | 5176 |
| pegRNA | 5177 |
| pegRNA | 5178 |
| pegRNA | 5179 |
| pegRNA | 5180 |
| pegRNA | 5181 |
| pegRNA | 5182 |
| pegRNA | 5183 |
| pegRNA | 5184 |
| pegRNA | 5185 |
| pegRNA | 5186 |
| pegRNA | 5187 |
| pegRNA | 5188 |
| pegRNA | 5189 |
| pegRNA | 5190 |
| pegRNA | 5191 |
| pegRNA | 5192 |
| pegRNA | 5193 |
| pegRNA | 5194 |
| pegRNA | 5195 |
| pegRNA | 5196 |
| pegRNA | 5197 |
| pegRNA | 5198 |
| pegRNA | 5199 |
| pegRNA | 5200 |
| pegRNA | 5201 |
| pegRNA | 5202 |
| pegRNA | 5203 |
| pegRNA | 5204 |
| pegRNA | 5205 |
| ngRNA | 2267 |
| ngRNA | 2257 |
| ngRNA | 2277 |
| ngRNA | 2276 |
| ngRNA | 2262 |
| ngRNA | 4418 |
| ngRNA | 2270 |
| ngRNA | 2274 |
| ngRNA | 2264 |
| ngRNA | 2268 |
| ngRNA | 2275 |
| ngRNA | 2272 |
| ngRNA | 2271 |
| ngRNA | 2265 |
| ngRNA | 2263 |
| ngRNA | 2266 |
| ngRNA | 2273 |
| ngRNA | 4411 |
| ngRNA | 2269 |
| ngRNA | 4416 |
| ngRNA | 2258 |
| ngRNA | 2261 |
| ngRNA | 2260 |
| ngRNA | 2259 |
| ngRNA | 2278 |
| ngRNA | 2284 |
| ngRNA | 2282 |
| ngRNA | 2288 |
| ngRNA | 4420 |
| ngRNA | 2285 |
| ngRNA | 2283 |
| ngRNA | 2280 |
| ngRNA | 4422 |
| ngRNA | 2281 |
| ngRNA | 2287 |
| ngRNA | 2279 |
| ngRNA | 2286 |
| ngRNA | 2289 |
| ngRNA | 2291 |
| ngRNA | 2290 |
| ngRNA | 2292 |

TABLE 17

| Sequence region | SEQ ID |
|---|---|
| pegRNA Spacer | 5206 |
| PBS | 5207 |
| PBS | 5208 |
| PBS | 5209 |
| PBS | 5210 |
| PBS | 5211 |
| PBS | 5212 |
| PBS | 5213 |
| PBS | 5214 |
| PBS | 5215 |
| PBS | 5216 |
| PBS | 5217 |
| RTT | 5218 |
| ngRNA Spacer | 199 |
| ngRNA Spacer | 24 |
| ngRNA Spacer | 20 |
| ngRNA Spacer | 200 |
| ngRNA Spacer | 52 |
| ngRNA Spacer | 21 |
| ngRNA Spacer | 56 |
| ngRNA Spacer | 57 |
| ngRNA Spacer | 51 |
| ngRNA Spacer | 45 |
| ngRNA Spacer | 5219 |
| ngRNA Spacer | 27 |
| ngRNA Spacer | 55 |
| ngRNA Spacer | 737 |
| ngRNA Spacer | 18 |
| ngRNA Spacer | 32 |
| ngRNA Spacer | 72 |
| ngRNA Spacer | 529 |
| ngRNA Spacer | 43 |
| ngRNA Spacer | 23 |
| ngRNA Spacer | 31 |
| ngRNA Spacer | 22 |
| ngRNA Spacer | 34 |
| ngRNA Spacer | 30 |
| ngRNA Spacer | 35 |
| ngRNA Spacer | 46 |
| ngRNA Spacer | 40 |
| ngRNA Spacer | 33 |
| ngRNA Spacer | 39 |
| ngRNA Spacer | 58 |
| ngRNA Spacer | 25 |
| ngRNA Spacer | 28 |
| ngRNA Spacer | 29 |
| ngRNA Spacer | 47 |
| ngRNA Spacer | 5220 |
| ngRNA Spacer | 68 |
| ngRNA Spacer | 37 |
| ngRNA Spacer | 67 |
| ngRNA Spacer | 44 |
| ngRNA Spacer | 19 |
| ngRNA Spacer | 38 |
| ngRNA Spacer | 70 |
| ngRNA Spacer | 42 |
| ngRNA Spacer | 59 |
| ngRNA Spacer | 48 |
| ngRNA Spacer | 36 |
| ngRNA Spacer | 49 |
| ngRNA Spacer | 54 |
| ngRNA Spacer | 5221 |
| ngRNA Spacer | 5222 |
| ngRNA Spacer | 5223 |
| ngRNA Spacer | 5224 |
| ngRNA Spacer | 5225 |
| ngRNA Spacer | 5226 |
| ngRNA Spacer | 5227 |
| ngRNA Spacer | 26 |
| ngRNA Spacer | 53 |
| ngRNA Spacer | 203 |
| ngRNA Spacer | 50 |
| ngRNA | 155 |
| ngRNA | 163 |
| ngRNA | 156 |
| ngRNA | 154 |
| ngRNA | 167 |
| ngRNA | 153 |
| ngRNA | 157 |
| ngRNA | 169 |
| ngRNA | 168 |
| ngRNA | 290 |
| ngRNA | 159 |
| ngRNA | 160 |
| ngRNA | 291 |
| ngRNA | 1501 |
| ngRNA | 161 |
| ngRNA | 681 |
| ngRNA | 164 |
| ngRNA | 162 |
| ngRNA | 166 |
| ngRNA | 165 |
| ngRNA | 158 |
| ngRNA | 171 |
| ngRNA | 170 |
| ngRNA | 173 |
| ngRNA | 293 |
| ngRNA | 179 |
| ngRNA | 1504 |
| ngRNA | 180 |
| ngRNA | 176 |
| ngRNA | 175 |
| ngRNA | 181 |
| ngRNA | 178 |
| ngRNA | 292 |
| ngRNA | 177 |
| ngRNA | 172 |
| ngRNA | 174 |

TABLE 18

| Sequence region | SEQ ID |
|---|---|
| pegRNA Spacer | 5228 |
| PBS | 5229 |
| PBS | 5230 |
| PBS | 5231 |
| PBS | 5232 |
| PBS | 5233 |
| PBS | 5234 |
| PBS | 5235 |
| PBS | 5236 |
| PBS | 5237 |
| PBS | 5238 |
| PBS | 5239 |
| RTT | 5240 |
| RTT | 5241 |
| RTT | 5242 |
| RTT | 5243 |
| RTT | 5244 |
| RTT | 5245 |
| RTT | 5246 |
| RTT | 5247 |
| ngRNA Spacer | 28 |
| ngRNA Spacer | 55 |
| ngRNA Spacer | 35 |
| ngRNA Spacer | 737 |
| ngRNA Spacer | 736 |
| ngRNA Spacer | 529 |
| ngRNA Spacer | 43 |
| ngRNA Spacer | 200 |
| ngRNA Spacer | 203 |
| ngRNA Spacer | 199 |
| ngRNA Spacer | 34 |
| ngRNA Spacer | 5219 |
| ngRNA Spacer | 740 |
| ngRNA Spacer | 33 |
| ngRNA Spacer | 37 |
| ngRNA Spacer | 48 |
| ngRNA Spacer | 20 |
| ngRNA Spacer | 19 |
| ngRNA Spacer | 38 |
| ngRNA Spacer | 46 |
| ngRNA Spacer | 738 |
| ngRNA Spacer | 30 |
| ngRNA Spacer | 50 |
| ngRNA Spacer | 24 |
| ngRNA Spacer | 23 |
| ngRNA Spacer | 47 |
| ngRNA Spacer | 22 |
| ngRNA Spacer | 29 |
| ngRNA Spacer | 70 |
| ngRNA Spacer | 58 |
| ngRNA Spacer | 52 |
| ngRNA Spacer | 51 |
| ngRNA Spacer | 49 |
| ngRNA Spacer | 45 |
| ngRNA Spacer | 68 |
| ngRNA Spacer | 59 |
| ngRNA Spacer | 31 |
| ngRNA Spacer | 18 |
| ngRNA Spacer | 67 |
| ngRNA Spacer | 53 |
| ngRNA Spacer | 5220 |
| ngRNA Spacer | 21 |
| ngRNA Spacer | 26 |
| ngRNA Spacer | 36 |
| ngRNA Spacer | 56 |
| ngRNA Spacer | 32 |
| ngRNA Spacer | 44 |
| ngRNA Spacer | 5221 |
| ngRNA Spacer | 739 |
| ngRNA Spacer | 39 |
| ngRNA Spacer | 40 |
| ngRNA Spacer | 42 |
| ngRNA Spacer | 72 |
| ngRNA Spacer | 54 |
| ngRNA Spacer | 57 |
| ngRNA Spacer | 5227 |
| ngRNA Spacer | 27 |
| ngRNA Spacer | 5225 |
| ngRNA Spacer | 5224 |
| ngRNA Spacer | 5226 |
| ngRNA Spacer | 5223 |
| ngRNA Spacer | 5222 |
| ngRNA Spacer | 25 |
| ngRNA | 162 |
| ngRNA | 164 |
| ngRNA | 160 |
| ngRNA | 1503 |
| ngRNA | 681 |
| ngRNA | 154 |
| ngRNA | 155 |
| ngRNA | 156 |
| ngRNA | 290 |
| ngRNA | 153 |
| ngRNA | 166 |
| ngRNA | 1501 |
| ngRNA | 291 |
| ngRNA | 158 |
| ngRNA | 163 |
| ngRNA | 157 |
| ngRNA | 169 |
| ngRNA | 167 |
| ngRNA | 168 |
| ngRNA | 159 |
| ngRNA | 161 |
| ngRNA | 1502 |
| ngRNA | 165 |
| ngRNA | 1504 |
| ngRNA | 180 |
| ngRNA | 292 |
| ngRNA | 178 |
| ngRNA | 179 |
| ngRNA | 170 |
| ngRNA | 172 |
| ngRNA | 173 |
| ngRNA | 177 |
| ngRNA | 293 |
| ngRNA | 176 |
| ngRNA | 174 |
| ngRNA | 175 |
| ngRNA | 181 |
| ngRNA | 171 |

TABLE 19

| Sequence region | SEQ ID |
|---|---|
| pegRNA Spacer | 5248 |
| PBS | 5249 |
| PBS | 5250 |
| PBS | 5251 |
| PBS | 5252 |
| PBS | 5253 |
| PBS | 5254 |
| PBS | 5255 |
| PBS | 5256 |
| PBS | 5257 |
| PBS | 5258 |
| PBS | 5259 |
| RTT | 5260 |
| RTT | 5261 |
| RTT | 5262 |
| RTT | 5263 |
| RTT | 5264 |
| RTT | 5265 |
| RTT | 5266 |
| RTT | 5267 |
| RTT | 5268 |
| RTT | 5269 |
| RTT | 5270 |
| RTT | 5271 |
| RTT | 5272 |
| RTT | 5273 |
| RTT | 5274 |
| RTT | 5275 |
| RTT | 5276 |
| RTT | 5277 |
| RTT | 5278 |
| RTT | 5279 |
| ngRNA Spacer | 40 |
| ngRNA Spacer | 738 |
| ngRNA Spacer | 529 |
| ngRNA Spacer | 44 |
| ngRNA Spacer | 29 |
| ngRNA Spacer | 26 |
| ngRNA Spacer | 42 |
| ngRNA Spacer | 5280 |
| ngRNA Spacer | 30 |
| ngRNA Spacer | 50 |
| ngRNA Spacer | 46 |
| ngRNA Spacer | 5219 |
| ngRNA Spacer | 25 |
| ngRNA Spacer | 52 |
| ngRNA Spacer | 737 |
| ngRNA Spacer | 55 |
| ngRNA Spacer | 49 |
| ngRNA Spacer | 48 |
| ngRNA Spacer | 5281 |
| ngRNA Spacer | 740 |
| ngRNA Spacer | 739 |
| ngRNA Spacer | 57 |
| ngRNA Spacer | 203 |
| ngRNA Spacer | 43 |
| ngRNA Spacer | 200 |
| ngRNA Spacer | 33 |
| ngRNA Spacer | 24 |
| ngRNA Spacer | 54 |
| ngRNA Spacer | 45 |
| ngRNA Spacer | 21 |
| ngRNA Spacer | 47 |
| ngRNA Spacer | 37 |
| ngRNA Spacer | 19 |
| ngRNA Spacer | 58 |
| ngRNA Spacer | 32 |
| ngRNA Spacer | 59 |
| ngRNA Spacer | 5220 |
| ngRNA Spacer | 56 |
| ngRNA Spacer | 736 |
| ngRNA Spacer | 67 |
| ngRNA Spacer | 28 |
| ngRNA Spacer | 39 |
| ngRNA Spacer | 20 |
| ngRNA Spacer | 70 |
| ngRNA Spacer | 199 |
| ngRNA Spacer | 22 |
| ngRNA Spacer | 68 |
| ngRNA Spacer | 5221 |
| ngRNA Spacer | 18 |
| ngRNA Spacer | 36 |
| ngRNA Spacer | 27 |
| ngRNA Spacer | 31 |
| ngRNA Spacer | 23 |
| ngRNA Spacer | 53 |
| ngRNA Spacer | 35 |
| ngRNA Spacer | 38 |
| ngRNA Spacer | 34 |
| ngRNA Spacer | 72 |
| ngRNA Spacer | 5227 |
| ngRNA Spacer | 5225 |
| ngRNA Spacer | 5224 |
| ngRNA Spacer | 5226 |
| ngRNA Spacer | 5223 |
| ngRNA Spacer | 51 |
| ngRNA Spacer | 5222 |
| ngRNA | 164 |
| ngRNA | 162 |
| ngRNA | 160 |
| ngRNA | 1503 |
| ngRNA | 161 |
| ngRNA | 153 |
| ngRNA | 1502 |
| ngRNA | 290 |
| ngRNA | 168 |
| ngRNA | 167 |
| ngRNA | 169 |
| ngRNA | 157 |
| ngRNA | 165 |
| ngRNA | 158 |
| ngRNA | 166 |
| ngRNA | 291 |
| ngRNA | 154 |
| ngRNA | 156 |
| ngRNA | 163 |
| ngRNA | 159 |
| ngRNA | 681 |
| ngRNA | 155 |
| ngRNA | 1501 |
| ngRNA | 174 |
| ngRNA | 176 |
| ngRNA | 181 |
| ngRNA | 179 |
| ngRNA | 175 |
| ngRNA | 292 |
| ngRNA | 1504 |
| ngRNA | 172 |
| ngRNA | 171 |
| ngRNA | 293 |
| ngRNA | 170 |
| ngRNA | 173 |
| ngRNA | 177 |
| ngRNA | 178 |
| ngRNA | 180 |

TABLE 20

| Sequence region | SEQ ID |
|---|---|
| pegRNA Spacer | 5282 |
| PBS | 5283 |
| PBS | 5284 |
| PBS | 5285 |
| PBS | 5286 |
| PBS | 5287 |
| PBS | 5288 |
| PBS | 5289 |
| PBS | 5290 |
| PBS | 5291 |
| PBS | 5292 |
| PBS | 5293 |
| RTT | 5294 |
| RTT | 5295 |
| RTT | 5296 |
| RTT | 5297 |
| RTT | 5298 |
| RTT | 5299 |
| RTT | 5300 |
| RTT | 5301 |
| RTT | 5302 |
| ngRNA Spacer | 202 |
| ngRNA Spacer | 5303 |
| ngRNA Spacer | 337 |
| ngRNA Spacer | 5304 |
| ngRNA Spacer | 5305 |
| ngRNA Spacer | 209 |
| ngRNA Spacer | 205 |
| ngRNA Spacer | 5306 |
| ngRNA Spacer | 204 |
| ngRNA Spacer | 740 |
| ngRNA Spacer | 70 |
| ngRNA Spacer | 33 |
| ngRNA Spacer | 35 |
| ngRNA Spacer | 206 |
| ngRNA Spacer | 50 |
| ngRNA Spacer | 5307 |
| ngRNA Spacer | 201 |
| ngRNA Spacer | 27 |
| ngRNA Spacer | 68 |
| ngRNA Spacer | 5308 |
| ngRNA Spacer | 30 |
| ngRNA Spacer | 5309 |
| ngRNA Spacer | 5310 |
| ngRNA Spacer | 200 |
| ngRNA Spacer | 736 |
| ngRNA Spacer | 207 |
| ngRNA Spacer | 208 |
| ngRNA Spacer | 5311 |
| ngRNA Spacer | 5312 |
| ngRNA | 1503 |
| ngRNA | 153 |
| ngRNA | 291 |
| ngRNA | 154 |
| ngRNA | 155 |
| ngRNA | 167 |
| ngRNA | 157 |
| ngRNA | 165 |
| ngRNA | 163 |
| ngRNA | 166 |
| ngRNA | 1501 |
| ngRNA | 160 |
| ngRNA | 162 |
| ngRNA | 173 |
| ngRNA | 172 |
| ngRNA | 171 |
| ngRNA | 170 |
| ngRNA | 178 |
| ngRNA | 1504 |
| ngRNA | 177 |
| ngRNA | 180 |
| ngRNA | 292 |
| ngRNA | 181 |

TABLE 21

| Sequence region | SEQ ID |
|---|---|
| pegRNA Spacer | 5313 |
| PBS | 5314 |
| PBS | 5315 |
| PBS | 5316 |
| PBS | 5317 |
| PBS | 5318 |
| PBS | 5319 |
| PBS | 5320 |
| PBS | 5321 |
| PBS | 5322 |
| PBS | 5323 |
| PBS | 5324 |
| RTT | 5325 |
| RTT | 5326 |
| RTT | 5327 |
| RTT | 5328 |
| RTT | 5329 |
| RTT | 5330 |
| RTT | 5331 |
| RTT | 5332 |
| RTT | 5333 |
| RTT | 5334 |
| RTT | 5335 |
| RTT | 5336 |
| RTT | 5337 |
| RTT | 5338 |
| ngRNA Spacer | 5312 |
| ngRNA Spacer | 5310 |
| ngRNA Spacer | 5308 |
| ngRNA Spacer | 207 |
| ngRNA Spacer | 5309 |
| ngRNA Spacer | 208 |
| ngRNA Spacer | 5303 |
| ngRNA Spacer | 5305 |
| ngRNA Spacer | 5311 |
| ngRNA Spacer | 201 |
| ngRNA Spacer | 5339 |
| ngRNA Spacer | 206 |
| ngRNA Spacer | 337 |
| ngRNA Spacer | 5307 |
| ngRNA | 160 |
| ngRNA | 165 |
| ngRNA | 166 |
| ngRNA | 155 |
| ngRNA | 291 |
| ngRNA | 171 |
| ngRNA | 177 |
| ngRNA | 172 |
| ngRNA | 292 |

TABLE 22

| Sequence region | SEQ ID |
|---|---|
| pegRNA Spacer | 5340 |
| PBS | 5341 |
| PBS | 5342 |
| PBS | 5343 |
| PBS | 5344 |
| PBS | 5345 |
| PBS | 5346 |
| PBS | 5347 |
| PBS | 5348 |
| PBS | 5349 |
| PBS | 5350 |
| PBS | 5351 |
| RTT | 5352 |
| RTT | 5353 |
| RTT | 5354 |
| RTT | 5355 |
| RTT | 5356 |
| RTT | 5357 |
| RTT | 5358 |
| RTT | 5359 |
| RTT | 5360 |
| RTT | 5361 |
| RTT | 5362 |
| RTT | 5363 |
| RTT | 5364 |
| RTT | 5365 |
| RTT | 5366 |
| RTT | 5367 |
| RTT | 5368 |
| ngRNA Spacer | 337 |
| ngRNA Spacer | 207 |
| ngRNA Spacer | 5305 |
| ngRNA Spacer | 5303 |
| ngRNA Spacer | 208 |
| ngRNA Spacer | 5307 |
| ngRNA Spacer | 5312 |
| ngRNA Spacer | 5339 |
| ngRNA Spacer | 206 |
| ngRNA Spacer | 5308 |
| ngRNA Spacer | 201 |
| ngRNA Spacer | 5311 |
| ngRNA Spacer | 5310 |
| ngRNA Spacer | 5309 |
| ngRNA | 160 |
| ngRNA | 165 |
| ngRNA | 166 |
| ngRNA | 155 |
| ngRNA | 291 |
| ngRNA | 171 |
| ngRNA | 292 |
| ngRNA | 172 |
| ngRNA | 177 |

TABLE 23

| Sequence region | SEQ ID |
|---|---|
| pegRNA Spacer | 5369 |
| PBS | 5370 |
| PBS | 5371 |
| PBS | 5372 |
| PBS | 5373 |
| PBS | 5374 |
| PBS | 5375 |
| PBS | 5376 |
| PBS | 5377 |
| PBS | 5378 |
| PBS | 5379 |
| PBS | 5380 |
| RTT | 5381 |
| RTT | 5382 |
| RTT | 5383 |
| RTT | 5384 |
| RTT | 5385 |
| RTT | 5386 |
| RTT | 5387 |
| RTT | 5388 |
| RTT | 5389 |
| RTT | 5390 |
| RTT | 5391 |
| RTT | 5392 |
| RTT | 5393 |
| RTT | 5394 |
| RTT | 5395 |
| RTT | 5396 |
| RTT | 5397 |
| RTT | 5398 |
| RTT | 5399 |
| RTT | 5400 |
| RTT | 5401 |
| ngRNA Spacer | 2434 |
| ngRNA Spacer | 2080 |
| ngRNA Spacer | 5402 |
| ngRNA Spacer | 2430 |
| ngRNA Spacer | 2436 |
| ngRNA Spacer | 5403 |
| ngRNA Spacer | 5404 |
| ngRNA Spacer | 2425 |
| ngRNA Spacer | 2052 |
| ngRNA Spacer | 2424 |
| ngRNA Spacer | 2088 |
| ngRNA Spacer | 5405 |
| ngRNA Spacer | 2426 |
| ngRNA Spacer | 2443 |
| ngRNA Spacer | 2427 |
| ngRNA Spacer | 2440 |
| ngRNA Spacer | 2048 |
| ngRNA Spacer | 2089 |
| ngRNA Spacer | 2439 |
| ngRNA Spacer | 2429 |
| ngRNA Spacer | 2435 |
| ngRNA Spacer | 2428 |
| ngRNA Spacer | 2444 |
| ngRNA Spacer | 2433 |
| ngRNA Spacer | 2070 |
| ngRNA Spacer | 2431 |
| ngRNA Spacer | 2086 |
| ngRNA Spacer | 2438 |
| ngRNA Spacer | 2432 |
| ngRNA Spacer | 2085 |
| ngRNA Spacer | 2423 |
| ngRNA Spacer | 2091 |
| ngRNA | 4412 |
| ngRNA | 4416 |
| ngRNA | 2270 |
| ngRNA | 2274 |
| ngRNA | 2262 |
| ngRNA | 2275 |
| ngRNA | 2272 |
| ngRNA | 4413 |
| ngRNA | 4415 |
| ngRNA | 2276 |
| ngRNA | 4417 |
| ngRNA | 2259 |
| ngRNA | 2260 |
| ngRNA | 2261 |
| ngRNA | 2257 |
| ngRNA | 4411 |
| ngRNA | 2264 |
| ngRNA | 2278 |
| ngRNA | 2282 |
| ngRNA | 2285 |
| ngRNA | 2283 |
| ngRNA | 2284 |
| ngRNA | 2288 |
| ngRNA | 4421 |
| ngRNA | 4419 |
| ngRNA | 2280 |
| ngRNA | 4420 |
| ngRNA | 2292 |
| ngRNA | 4424 |

TABLE 24

| Sequence region | SEQ ID |
|---|---|
| pegRNA Spacer | 5406 |
| PBS | 5407 |
| PBS | 5408 |
| PBS | 5409 |
| PBS | 5410 |
| PBS | 5411 |
| PBS | 5412 |
| PBS | 5413 |
| PBS | 5414 |
| PBS | 5415 |
| PBS | 5416 |
| PBS | 5417 |
| RTT | 5418 |
| RTT | 5419 |
| RTT | 5420 |
| RTT | 5421 |
| RTT | 5422 |
| ngRNA Spacer | 736 |
| ngRNA Spacer | 55 |
| ngRNA Spacer | 737 |
| ngRNA Spacer | 51 |
| ngRNA Spacer | 28 |
| ngRNA Spacer | 199 |
| ngRNA Spacer | 529 |
| ngRNA Spacer | 70 |
| ngRNA Spacer | 68 |
| ngRNA Spacer | 38 |
| ngRNA Spacer | 740 |
| ngRNA Spacer | 37 |
| ngRNA Spacer | 18 |
| ngRNA Spacer | 57 |
| ngRNA Spacer | 20 |
| ngRNA Spacer | 58 |
| ngRNA Spacer | 54 |
| ngRNA Spacer | 56 |
| ngRNA Spacer | 29 |
| ngRNA Spacer | 67 |
| ngRNA Spacer | 31 |
| ngRNA Spacer | 35 |
| ngRNA Spacer | 33 |
| ngRNA Spacer | 25 |
| ngRNA Spacer | 44 |
| ngRNA Spacer | 21 |
| ngRNA Spacer | 52 |
| ngRNA Spacer | 39 |
| ngRNA Spacer | 34 |
| ngRNA Spacer | 36 |
| ngRNA Spacer | 27 |
| ngRNA Spacer | 32 |
| ngRNA Spacer | 203 |
| ngRNA Spacer | 23 |
| ngRNA Spacer | 48 |
| ngRNA Spacer | 22 |
| ngRNA Spacer | 47 |
| ngRNA Spacer | 40 |
| ngRNA Spacer | 45 |
| ngRNA Spacer | 43 |
| ngRNA Spacer | 42 |
| ngRNA Spacer | 49 |
| ngRNA Spacer | 24 |
| ngRNA Spacer | 26 |
| ngRNA Spacer | 72 |
| ngRNA Spacer | 19 |
| ngRNA Spacer | 50 |
| ngRNA Spacer | 53 |
| ngRNA Spacer | 739 |
| ngRNA Spacer | 200 |
| ngRNA Spacer | 59 |
| ngRNA Spacer | 30 |
| ngRNA Spacer | 46 |
| ngRNA | 162 |
| ngRNA | 681 |
| ngRNA | 154 |
| ngRNA | 161 |
| ngRNA | 290 |
| ngRNA | 156 |
| ngRNA | 155 |
| ngRNA | 160 |
| ngRNA | 159 |
| ngRNA | 168 |
| ngRNA | 167 |
| ngRNA | 169 |
| ngRNA | 1503 |
| ngRNA | 165 |
| ngRNA | 158 |
| ngRNA | 166 |
| ngRNA | 1501 |
| ngRNA | 291 |
| ngRNA | 153 |
| ngRNA | 157 |
| ngRNA | 164 |
| ngRNA | 163 |
| ngRNA | 176 |
| ngRNA | 170 |
| ngRNA | 171 |
| ngRNA | 175 |
| ngRNA | 181 |
| ngRNA | 172 |
| ngRNA | 179 |
| ngRNA | 173 |
| ngRNA | 292 |
| ngRNA | 178 |
| ngRNA | 180 |
| ngRNA | 1504 |
| ngRNA | 293 |
| ngRNA | 177 |
| ngRNA | 174 |

TABLE 25

| Sequence region | SEQ ID |
|---|---|
| pegRNA Spacer | 5423 |
| PBS | 5424 |
| PBS | 5425 |
| PBS | 5426 |
| PBS | 5427 |
| PBS | 5428 |
| PBS | 5429 |
| PBS | 5430 |
| PBS | 5431 |
| PBS | 5432 |
| PBS | 5433 |
| PBS | 5434 |
| RTT | 5435 |
| RTT | 5436 |
| RTT | 5437 |
| RTT | 5438 |
| RTT | 5439 |
| RTT | 5440 |
| RTT | 5441 |
| RTT | 5442 |
| RTT | 5443 |
| RTT | 5444 |
| RTT | 5445 |
| ngRNA Spacer | 200 |
| ngRNA Spacer | 53 |
| ngRNA Spacer | 736 |
| ngRNA Spacer | 49 |
| ngRNA Spacer | 23 |
| ngRNA Spacer | 740 |
| ngRNA Spacer | 30 |
| ngRNA Spacer | 203 |
| ngRNA Spacer | 32 |
| ngRNA Spacer | 5280 |
| ngRNA Spacer | 22 |
| ngRNA Spacer | 35 |
| ngRNA Spacer | 45 |
| ngRNA Spacer | 26 |
| ngRNA Spacer | 70 |
| ngRNA Spacer | 737 |
| ngRNA Spacer | 59 |
| ngRNA Spacer | 44 |
| ngRNA Spacer | 46 |
| ngRNA Spacer | 37 |
| ngRNA Spacer | 56 |
| ngRNA Spacer | 58 |
| ngRNA Spacer | 20 |
| ngRNA Spacer | 21 |
| ngRNA Spacer | 67 |
| ngRNA Spacer | 38 |
| ngRNA Spacer | 738 |
| ngRNA Spacer | 47 |
| ngRNA Spacer | 34 |
| ngRNA Spacer | 68 |
| ngRNA Spacer | 52 |
| ngRNA Spacer | 54 |
| ngRNA Spacer | 51 |
| ngRNA Spacer | 739 |
| ngRNA Spacer | 43 |
| ngRNA Spacer | 18 |
| ngRNA Spacer | 42 |
| ngRNA Spacer | 39 |
| ngRNA Spacer | 72 |
| ngRNA Spacer | 57 |
| ngRNA Spacer | 55 |
| ngRNA Spacer | 25 |
| ngRNA Spacer | 27 |
| ngRNA Spacer | 33 |
| ngRNA Spacer | 29 |
| ngRNA Spacer | 31 |
| ngRNA Spacer | 40 |
| ngRNA Spacer | 50 |
| ngRNA Spacer | 19 |
| ngRNA Spacer | 24 |
| ngRNA Spacer | 529 |
| ngRNA Spacer | 28 |
| ngRNA Spacer | 36 |
| ngRNA Spacer | 48 |
| ngRNA Spacer | 199 |
| ngRNA | 681 |
| ngRNA | 154 |
| ngRNA | 158 |
| ngRNA | 1503 |
| ngRNA | 161 |
| ngRNA | 159 |
| ngRNA | 156 |
| ngRNA | 164 |
| ngRNA | 160 |
| ngRNA | 290 |
| ngRNA | 168 |
| ngRNA | 167 |
| ngRNA | 155 |
| ngRNA | 162 |
| ngRNA | 169 |
| ngRNA | 163 |
| ngRNA | 166 |
| ngRNA | 153 |
| ngRNA | 1501 |
| ngRNA | 291 |
| ngRNA | 157 |
| ngRNA | 1502 |
| ngRNA | 165 |
| ngRNA | 181 |
| ngRNA | 170 |
| ngRNA | 171 |
| ngRNA | 174 |
| ngRNA | 179 |
| ngRNA | 172 |
| ngRNA | 175 |
| ngRNA | 173 |
| ngRNA | 292 |
| ngRNA | 178 |
| ngRNA | 180 |
| ngRNA | 176 |
| ngRNA | 293 |
| ngRNA | 1504 |
| ngRNA | 177 |

TABLE 26

| Sequence region | SEQ ID |
|---|---|
| pegRNA Spacer | 5446 |
| PBS | 5447 |
| PBS | 5448 |
| PBS | 5449 |
| PBS | 5450 |
| PBS | 5451 |
| PBS | 5452 |
| PBS | 5453 |
| PBS | 5454 |
| PBS | 5455 |
| PBS | 5456 |
| PBS | 5457 |
| RTT | 5458 |
| RTT | 5459 |
| RTT | 5460 |
| RTT | 5461 |
| RTT | 5462 |
| RTT | 5463 |
| RTT | 5464 |
| RTT | 5465 |
| RTT | 5466 |
| RTT | 5467 |
| RTT | 5468 |
| RTT | 5469 |
| RTT | 5470 |
| RTT | 5471 |
| RTT | 5472 |
| ngRNA Spacer | 59 |
| ngRNA Spacer | 25 |
| ngRNA Spacer | 740 |
| ngRNA Spacer | 28 |
| ngRNA Spacer | 22 |
| ngRNA Spacer | 54 |
| ngRNA Spacer | 21 |
| ngRNA Spacer | 5280 |
| ngRNA Spacer | 5281 |
| ngRNA Spacer | 56 |
| ngRNA Spacer | 52 |
| ngRNA Spacer | 738 |
| ngRNA Spacer | 34 |
| ngRNA Spacer | 38 |
| ngRNA Spacer | 24 |
| ngRNA Spacer | 49 |
| ngRNA Spacer | 739 |
| ngRNA Spacer | 55 |
| ngRNA Spacer | 43 |
| ngRNA Spacer | 58 |
| ngRNA Spacer | 46 |
| ngRNA Spacer | 737 |
| ngRNA Spacer | 37 |
| ngRNA Spacer | 40 |
| ngRNA Spacer | 31 |
| ngRNA Spacer | 70 |
| ngRNA Spacer | 47 |
| ngRNA Spacer | 33 |
| ngRNA Spacer | 736 |
| ngRNA Spacer | 203 |
| ngRNA Spacer | 51 |
| ngRNA Spacer | 68 |
| ngRNA Spacer | 50 |
| ngRNA Spacer | 72 |
| ngRNA Spacer | 29 |
| ngRNA Spacer | 20 |
| ngRNA Spacer | 36 |
| ngRNA Spacer | 30 |
| ngRNA Spacer | 529 |
| ngRNA Spacer | 18 |
| ngRNA Spacer | 27 |
| ngRNA Spacer | 48 |
| ngRNA Spacer | 199 |
| ngRNA Spacer | 35 |
| ngRNA Spacer | 57 |
| ngRNA Spacer | 200 |
| ngRNA Spacer | 53 |
| ngRNA Spacer | 39 |
| ngRNA Spacer | 23 |
| ngRNA Spacer | 201 |
| ngRNA Spacer | 19 |
| ngRNA Spacer | 44 |
| ngRNA Spacer | 32 |
| ngRNA Spacer | 26 |
| ngRNA Spacer | 67 |
| ngRNA Spacer | 45 |
| ngRNA Spacer | 42 |
| ngRNA | 158 |
| ngRNA | 154 |
| ngRNA | 164 |
| ngRNA | 1503 |
| ngRNA | 161 |
| ngRNA | 155 |
| ngRNA | 156 |
| ngRNA | 1502 |
| ngRNA | 160 |
| ngRNA | 159 |
| ngRNA | 290 |
| ngRNA | 168 |
| ngRNA | 167 |
| ngRNA | 162 |
| ngRNA | 163 |
| ngRNA | 165 |
| ngRNA | 166 |
| ngRNA | 1501 |
| ngRNA | 153 |
| ngRNA | 29 |
| ngRNA | 169 |
| ngRNA | 681 |
| ngRNA | 157 |
| ngRNA | 181 |
| ngRNA | 170 |
| ngRNA | 177 |
| ngRNA | 174 |
| ngRNA | 179 |
| ngRNA | 180 |
| ngRNA | 175 |
| ngRNA | 173 |
| ngRNA | 292 |
| ngRNA | 178 |
| ngRNA | 172 |
| ngRNA | 176 |
| ngRNA | 293 |
| ngRNA | 1504 |
| ngRNA | 171 |

TABLE 27

| Sequence region | SEQ ID |
|---|---|
| pegRNA Spacer | 5473 |
| PBS | 5474 |
| PBS | 5475 |
| PBS | 5476 |
| PBS | 5477 |
| PBS | 5478 |
| PBS | 5479 |
| PBS | 5480 |
| PBS | 5481 |
| PBS | 5482 |
| PBS | 5483 |
| PBS | 5484 |
| RTT | 5485 |
| RTT | 5486 |
| RTT | 5487 |
| RTT | 5488 |
| RTT | 5489 |
| RTT | 5490 |
| RTT | 5491 |
| RTT | 5492 |
| RTT | 5493 |
| RTT | 5494 |
| RTT | 5495 |
| RTT | 5496 |
| RTT | 5497 |
| RTT | 5498 |
| RTT | 5499 |
| RTT | 5500 |
| RTT | 5501 |
| RTT | 5502 |
| ngRNA Spacer | 200 |
| ngRNA Spacer | 737 |
| ngRNA Spacer | 28 |
| ngRNA Spacer | 739 |
| ngRNA Spacer | 45 |
| ngRNA Spacer | 46 |
| ngRNA Spacer | 30 |
| ngRNA Spacer | 50 |
| ngRNA Spacer | 29 |
| ngRNA Spacer | 22 |
| ngRNA Spacer | 47 |
| ngRNA Spacer | 736 |
| ngRNA Spacer | 26 |
| ngRNA Spacer | 58 |
| ngRNA Spacer | 203 |
| ngRNA Spacer | 18 |
| ngRNA Spacer | 59 |
| ngRNA Spacer | 39 |
| ngRNA Spacer | 5280 |
| ngRNA Spacer | 68 |
| ngRNA Spacer | 43 |
| ngRNA Spacer | 54 |
| ngRNA Spacer | 33 |
| ngRNA Spacer | 740 |
| ngRNA Spacer | 34 |
| ngRNA Spacer | 55 |
| ngRNA Spacer | 49 |
| ngRNA Spacer | 51 |
| ngRNA Spacer | 19 |
| ngRNA Spacer | 42 |
| ngRNA Spacer | 67 |
| ngRNA Spacer | 57 |
| ngRNA Spacer | 36 |
| ngRNA Spacer | 23 |
| ngRNA Spacer | 21 |
| ngRNA Spacer | 738 |
| ngRNA Spacer | 38 |
| ngRNA Spacer | 529 |
| ngRNA Spacer | 31 |
| ngRNA Spacer | 48 |
| ngRNA Spacer | 53 |
| ngRNA Spacer | 35 |
| ngRNA Spacer | 5281 |
| ngRNA Spacer | 25 |
| ngRNA Spacer | 52 |
| ngRNA Spacer | 27 |
| ngRNA Spacer | 70 |
| ngRNA Spacer | 40 |
| ngRNA Spacer | 32 |
| ngRNA Spacer | 20 |
| ngRNA Spacer | 24 |
| ngRNA Spacer | 37 |
| ngRNA Spacer | 199 |
| ngRNA Spacer | 56 |
| ngRNA Spacer | 72 |
| ngRNA Spacer | 44 |
| ngRNA | 162 |
| ngRNA | 161 |
| ngRNA | 154 |
| ngRNA | 164 |
| ngRNA | 1503 |
| ngRNA | 155 |
| ngRNA | 681 |
| ngRNA | 158 |
| ngRNA | 1502 |
| ngRNA | 160 |
| ngRNA | 159 |
| ngRNA | 290 |
| ngRNA | 168 |
| ngRNA | 156 |
| ngRNA | 167 |
| ngRNA | 157 |
| ngRNA | 163 |
| ngRNA | 166 |
| ngRNA | 153 |
| ngRNA | 1501 |
| ngRNA | 291 |
| ngRNA | 169 |
| ngRNA | 165 |
| ngRNA | 175 |
| ngRNA | 1504 |
| ngRNA | 176 |
| ngRNA | 179 |
| ngRNA | 177 |
| ngRNA | 170 |
| ngRNA | 180 |
| ngRNA | 171 |
| ngRNA | 173 |
| ngRNA | 293 |
| ngRNA | 172 |
| ngRNA | 174 |
| ngRNA | 178 |
| ngRNA | 292 |
| ngRNA | 181 |

TABLE 28

| Sequence region | SEQ ID |
|---|---|
| pegRNA Spacer | 5503 |
| PBS | 5504 |
| PBS | 5505 |
| PBS | 5506 |
| PBS | 5507 |
| PBS | 5508 |
| PBS | 5509 |
| PBS | 5510 |
| PBS | 5511 |
| PBS | 5512 |
| PBS | 5513 |
| PBS | 5514 |
| RTT | 5515 |
| RTT | 5516 |
| RTT | 5517 |
| RTT | 5518 |
| RTT | 5519 |
| RTT | 5520 |
| RTT | 5521 |
| RTT | 5522 |
| RTT | 5523 |
| RTT | 5524 |
| RTT | 5525 |
| RTT | 5526 |
| RTT | 5527 |
| RTT | 5528 |
| RTT | 5529 |
| RTT | 5530 |
| RTT | 5531 |
| RTT | 5532 |
| RTT | 5533 |
| RTT | 5534 |
| RTT | 5535 |
| ngRNA Spacer | 2052 |
| ngRNA Spacer | 2437 |
| ngRNA Spacer | 2096 |
| ngRNA Spacer | 2074 |
| ngRNA Spacer | 2066 |
| ngRNA Spacer | 2065 |
| ngRNA Spacer | 2094 |
| ngRNA Spacer | 2090 |
| ngRNA Spacer | 2444 |
| ngRNA Spacer | 2073 |
| ngRNA Spacer | 2077 |
| ngRNA Spacer | 2063 |
| ngRNA Spacer | 2046 |
| ngRNA Spacer | 2064 |
| ngRNA Spacer | 2089 |
| ngRNA Spacer | 2060 |
| ngRNA Spacer | 2091 |
| ngRNA Spacer | 2086 |
| ngRNA Spacer | 2093 |
| ngRNA Spacer | 2079 |
| ngRNA Spacer | 2054 |
| ngRNA Spacer | 2049 |
| ngRNA Spacer | 2443 |
| ngRNA Spacer | 2057 |
| ngRNA Spacer | 2069 |
| ngRNA Spacer | 2085 |
| ngRNA Spacer | 2059 |
| ngRNA Spacer | 2047 |
| ngRNA Spacer | 2071 |
| ngRNA Spacer | 2081 |
| ngRNA Spacer | 2072 |
| ngRNA Spacer | 2048 |
| ngRNA Spacer | 2068 |
| ngRNA Spacer | 2045 |
| ngRNA Spacer | 2078 |
| ngRNA Spacer | 5536 |
| ngRNA Spacer | 2088 |
| ngRNA Spacer | 2055 |
| ngRNA Spacer | 2084 |
| ngRNA Spacer | 2050 |
| ngRNA Spacer | 2092 |
| ngRNA Spacer | 2062 |
| ngRNA Spacer | 2087 |
| ngRNA Spacer | 2082 |
| ngRNA Spacer | 2061 |
| ngRNA Spacer | 2080 |
| ngRNA Spacer | 2095 |
| ngRNA Spacer | 2056 |
| ngRNA Spacer | 2083 |
| ngRNA Spacer | 2442 |
| ngRNA Spacer | 2075 |
| ngRNA Spacer | 2058 |
| ngRNA Spacer | 2067 |
| ngRNA Spacer | 2439 |
| ngRNA Spacer | 2051 |
| ngRNA Spacer | 2070 |
| ngRNA Spacer | 2076 |
| ngRNA Spacer | 2053 |
| ngRNA | 2270 |
| ngRNA | 2267 |
| ngRNA | 4416 |
| ngRNA | 2268 |
| ngRNA | 2263 |
| ngRNA | 2262 |
| ngRNA | 2265 |
| ngRNA | 2272 |
| ngRNA | 2275 |
| ngRNA | 4411 |
| ngRNA | 2276 |
| ngRNA | 2266 |
| ngRNA | 2258 |
| ngRNA | 2271 |
| ngRNA | 2261 |
| ngRNA | 2259 |
| ngRNA | 2274 |
| ngRNA | 2260 |
| ngRNA | 2257 |
| ngRNA | 2277 |
| ngRNA | 4418 |
| ngRNA | 2273 |
| ngRNA | 2264 |
| ngRNA | 2269 |
| ngRNA | 2279 |
| ngRNA | 2281 |
| ngRNA | 2285 |
| ngRNA | 2283 |
| ngRNA | 4420 |
| ngRNA | 2280 |
| ngRNA | 2286 |
| ngRNA | 2282 |
| ngRNA | 2284 |
| ngRNA | 2278 |
| ngRNA | 2288 |
| ngRNA | 2287 |
| ngRNA | 4422 |
| ngRNA | 2289 |
| ngRNA | 2291 |
| ngRNA | 2290 |
| ngRNA | 2292 |

TABLE 29

| Sequence region | SEQ ID |
|---|---|
| pegRNA Spacer | 5537 |
| PBS | 5538 |
| PBS | 5539 |
| PBS | 5540 |
| PBS | 5541 |
| PBS | 5542 |
| PBS | 5543 |
| PBS | 5544 |
| PBS | 5545 |
| PBS | 5546 |
| PBS | 5547 |
| PBS | 5548 |
| RTT | 5549 |
| RTT | 5550 |
| RTT | 5551 |
| RTT | 5552 |
| RTT | 5553 |
| RTT | 5554 |
| ngRNA Spacer | 70 |
| ngRNA Spacer | 30 |
| ngRNA Spacer | 204 |
| ngRNA Spacer | 68 |
| ngRNA Spacer | 209 |
| ngRNA Spacer | 206 |
| ngRNA Spacer | 208 |
| ngRNA Spacer | 201 |
| ngRNA Spacer | 35 |
| ngRNA Spacer | 337 |
| ngRNA Spacer | 27 |
| ngRNA Spacer | 202 |
| ngRNA Spacer | 200 |
| ngRNA Spacer | 736 |
| ngRNA Spacer | 33 |
| ngRNA Spacer | 5306 |
| ngRNA Spacer | 50 |
| ngRNA Spacer | 205 |
| ngRNA Spacer | 207 |
| ngRNA Spacer | 740 |
| ngRNA | 1503 |
| ngRNA | 153 |
| ngRNA | 157 |
| ngRNA | 162 |
| ngRNA | 167 |
| ngRNA | 165 |
| ngRNA | 291 |
| ngRNA | 163 |
| ngRNA | 166 |
| ngRNA | 1501 |
| ngRNA | 160 |
| ngRNA | 154 |
| ngRNA | 181 |
| ngRNA | 171 |
| ngRNA | 1504 |
| ngRNA | 292 |
| ngRNA | 173 |
| ngRNA | 170 |
| ngRNA | 172 |
| ngRNA | 180 |
| ngRNA | 178 |
| ngRNA | 177 |

TABLE 30

| Sequence region | SEQ ID |
|---|---|
| pegRNA Spacer | 5555 |
| PBS | 5556 |
| PBS | 5557 |
| PBS | 5558 |
| PBS | 5559 |
| PBS | 5560 |
| PBS | 5561 |
| PBS | 5562 |
| PBS | 5563 |
| PBS | 5564 |
| PBS | 5565 |
| PBS | 5566 |
| RTT | 5567 |
| RTT | 5568 |
| RTT | 5569 |
| RTT | 5570 |
| RTT | 5571 |
| RTT | 5572 |
| RTT | 5573 |
| RTT | 5574 |
| RTT | 5575 |
| RTT | 5576 |
| RTT | 5577 |
| RTT | 5578 |
| RTT | 5579 |
| RTT | 5580 |
| RTT | 5581 |
| RTT | 5582 |
| RTT | 5583 |
| RTT | 5584 |
| RTT | 5585 |
| RTT | 5586 |
| RTT | 5587 |
| RTT | 5588 |
| RTT | 5589 |
| RTT | 5590 |
| ngRNA Spacer | 65 |
| ngRNA Spacer | 64 |
| ngRNA Spacer | 63 |
| ngRNA Spacer | 61 |
| ngRNA Spacer | 62 |
| ngRNA Spacer | 69 |
| ngRNA Spacer | 66 |
| ngRNA Spacer | 60 |
| ngRNA Spacer | 41 |
| ngRNA Spacer | 71 |
| pegRNA | 5591 |
| pegRNA | 5592 |
| pegRNA | 5593 |
| pegRNA | 5594 |
| pegRNA | 5595 |
| pegRNA | 5596 |
| pegRNA | 5597 |
| pegRNA | 5598 |
| pegRNA | 5599 |
| pegRNA | 5600 |
| pegRNA | 5601 |
| pegRNA | 5602 |
| pegRNA | 5603 |
| pegRNA | 5604 |
| pegRNA | 5605 |
| pegRNA | 5606 |
| pegRNA | 5607 |
| pegRNA | 5608 |
| pegRNA | 5609 |
| pegRNA | 5610 |
| pegRNA | 5611 |
| pegRNA | 5612 |
| pegRNA | 5613 |
| pegRNA | 5614 |
| pegRNA | 5615 |
| pegRNA | 5616 |
| pegRNA | 5617 |
| pegRNA | 5618 |
| pegRNA | 5619 |
| pegRNA | 5620 |
| pegRNA | 5621 |
| pegRNA | 5622 |
| pegRNA | 5623 |
| pegRNA | 5624 |
| pegRNA | 5625 |
| pegRNA | 5626 |
| pegRNA | 5627 |
| pegRNA | 5628 |
| pegRNA | 5629 |
| pegRNA | 5630 |
| pegRNA | 5631 |
| pegRNA | 5632 |
| pegRNA | 5633 |
| pegRNA | 5634 |
| pegRNA | 5635 |
| pegRNA | 5636 |
| pegRNA | 5637 |

TABLE 31

| Sequence region | SEQ ID |
|---|---|
| pegRNA Spacer | 5638 |
| PBS | 5639 |
| PBS | 5640 |
| PBS | 5641 |
| PBS | 5642 |
| PBS | 5643 |
| PBS | 5644 |
| PBS | 5645 |
| PBS | 5646 |
| PBS | 5647 |
| PBS | 5648 |
| PBS | 5649 |
| RTT | 5650 |
| RTT | 5651 |
| RTT | 5652 |
| RTT | 5653 |
| RTT | 5654 |
| RTT | 5655 |
| RTT | 5656 |
| RTT | 5657 |
| RTT | 5658 |
| RTT | 5659 |
| RTT | 5660 |
| RTT | 5661 |
| RTT | 5662 |
| RTT | 5663 |
| RTT | 5664 |
| RTT | 5665 |
| RTT | 5666 |
| RTT | 5667 |
| RTT | 5668 |
| ngRNA Spacer | 65 |
| ngRNA Spacer | 64 |
| ngRNA Spacer | 62 |
| ngRNA Spacer | 61 |
| ngRNA Spacer | 63 |
| ngRNA Spacer | 69 |
| ngRNA Spacer | 66 |
| ngRNA Spacer | 60 |
| ngRNA Spacer | 41 |
| ngRNA Spacer | 71 |
| pegRNA | 5669 |
| pegRNA | 5670 |
| pegRNA | 5671 |
| pegRNA | 5672 |
| pegRNA | 5673 |
| pegRNA | 5674 |
| pegRNA | 5675 |
| pegRNA | 5676 |
| pegRNA | 5677 |
| pegRNA | 5678 |
| pegRNA | 5679 |
| pegRNA | 5680 |
| pegRNA | 5681 |
| pegRNA | 5682 |
| pegRNA | 5683 |
| pegRNA | 5684 |
| pegRNA | 5685 |
| pegRNA | 5686 |
| pegRNA | 5687 |
| pegRNA | 5688 |
| pegRNA | 5689 |
| pegRNA | 5690 |
| pegRNA | 5691 |
| pegRNA | 5692 |
| pegRNA | 5693 |
| pegRNA | 5694 |
| pegRNA | 5695 |
| pegRNA | 5696 |
| pegRNA | 5697 |
| pegRNA | 5698 |
| pegRNA | 5699 |
| pegRNA | 5700 |
| pegRNA | 5701 |
| pegRNA | 5702 |
| pegRNA | 5703 |
| pegRNA | 5704 |
| pegRNA | 5705 |

TABLE 32

| Sequence region | SEQ ID |
|---|---|
| pegRNA Spacer | 5706 |
| PBS | 5707 |
| PBS | 5708 |
| PBS | 5709 |
| PBS | 5710 |
| PBS | 5711 |
| PBS | 5712 |
| PBS | 5713 |
| PBS | 5714 |
| PBS | 5715 |
| PBS | 5716 |
| PBS | 5717 |
| RTT | 5718 |
| RTT | 5719 |
| RTT | 5720 |
| RTT | 5721 |
| RTT | 5722 |
| RTT | 5723 |
| RTT | 5724 |
| RTT | 5725 |
| RTT | 5726 |
| RTT | 5727 |
| RTT | 5728 |
| RTT | 5729 |
| RTT | 5730 |
| RTT | 5731 |
| RTT | 5732 |
| RTT | 5733 |
| RTT | 5734 |
| RTT | 5735 |
| RTT | 5736 |
| RTT | 5737 |
| RTT | 5738 |
| ngRNA Spacer | 65 |
| ngRNA Spacer | 64 |
| ngRNA Spacer | 62 |
| ngRNA Spacer | 61 |
| ngRNA Spacer | 63 |
| ngRNA Spacer | 69 |
| ngRNA Spacer | 66 |
| ngRNA Spacer | 60 |
| ngRNA Spacer | 41 |
| ngRNA Spacer | 71 |
| pegRNA | 5739 |
| pegRNA | 5740 |
| pegRNA | 5741 |
| pegRNA | 5742 |
| pegRNA | 5743 |
| pegRNA | 5744 |
| pegRNA | 5745 |
| pegRNA | 5746 |
| pegRNA | 5747 |
| pegRNA | 5748 |
| pegRNA | 5749 |
| pegRNA | 5750 |
| pegRNA | 5751 |
| pegRNA | 5752 |
| pegRNA | 5753 |
| pegRNA | 5754 |
| pegRNA | 5755 |
| pegRNA | 5756 |
| pegRNA | 5757 |
| pegRNA | 5758 |
| pegRNA | 5759 |
| pegRNA | 5760 |
| pegRNA | 5761 |
| pegRNA | 5762 |
| pegRNA | 5763 |
| pegRNA | 5764 |
| pegRNA | 5765 |
| pegRNA | 5766 |
| pegRNA | 5767 |
| pegRNA | 5768 |
| pegRNA | 5769 |
| pegRNA | 5770 |
| pegRNA | 5771 |
| pegRNA | 5772 |
| pegRNA | 5773 |
| pegRNA | 5774 |
| pegRNA | 5775 |
| pegRNA | 5776 |
| pegRNA | 5777 |
| pegRNA | 5778 |
| pegRNA | 5779 |

In some embodiments, a PEgRNA as described herein comprises a spacer comprising a PEgRNA spacer sequence as provided in Table x, a PBS comprising a PBS sequence as provided in Table x, and an editing template comprising an RTT sequence as provided in Table x, wherein for each PEgRNA, x is the same integer for the spacer, the PBS, and the editing template, and wherein x is an integer selected from 6, 7, 8, 9, 10, 11, 12, 15, 16, 17, 18, 19, 20, 21, 22, 23, 24, 25, 26, 27, 28, 29, 30, 31, and 32. In some embodiments, the PEgRNA is a part of a prime editing system, wherein the PEgRNA comprises a spacer comprising a PEgRNA spacer sequence as provided in Table x, a PBS comprising a PBS sequence as provided in Table x, and an editing template comprising an RTT sequence as provided in Table x, and wherein the prime editing system further comprises an ngRNA, wherein the ngRNA comprises a ngRNA spacer sequence as provided in Table x, wherein x is the same integer for the spacer, PBS, and editing template selection and for the ngRNA spacer selection, and wherein x is an integer selected from 6, 7, 8, 9, 10, 11, 12, 15, 16, 17, 18, 19, 20, 21, 22, 23, 24, 25, 26, 27, 28, 29, 30, 31 and 32.

In some embodiments, the PEgRNA and/or the ngRNA comprises a gRNA core, wherein the gRNA core comprises a sequence selected from SEQ ID Nos 5857-5859.

Table 6 provides Prime Editing guide RNAs (PEgRNAs), which can be used in Prime Editing systems disclosed herein. Such Prime Editing systems can comprise a Cas9 protein capable of recognizing an NGG or an NG PAM sequence (e.g., TGG or TG), and a reverse transcriptase. The Prime Editing systems (e.g., PE3 or PE3b systems) can further comprise a nick guide RNA (ngRNA). Such PEgRNAs and Prime Editing systems can be used, for example, to correct an H1069Q mutation in ATP7B.

The PEgRNAs of Table 6 comprise: (a) a spacer comprising nucleotides 5-20 of SEQ ID NO: 1, (b) a gRNA core capable of binding to a Cas9 protein, and (c) an extension arm comprising: (i) an editing template comprising any one of SEQ ID NOs: 13-17, and (ii) a primer binding site (PBS) comprising any one of SEQ ID NOs: 2-12. The spacer of the PEgRNA can comprise, for example, nucleotides 4-20, 3-20, 2-20, or 1-20 of SEQ ID NO: 1. The spacer of the PEgRNA can comprise SEQ ID NO: 1. The RTT and the PBS can comprise respectively SEQ ID NOs: 13 and 2, 13 and 3, 13 and 4, 13 and 5, 13 and 6, 13 and 7, 13 and 8, 13 and 9, 13 and 10, 13 and 11, 13 and 12, 14 and 2, 14 and 3, 14 and 4, 14 and 5, 14 and 6, 14 and 7, 14 and 8, 14 and 9, 14 and 10, 14 and 11, 14 and 12, 15 and 2, 15 and 3, 15 and 4, 15 and 5, 15 and 6, 15 and 7, 15 and 8, 15 and 9, 15 and 10, 15 and 11, 15 and 12, 16 and 2, 16 and 3, 16 and 4, 16 and 5, 16 and 6, 16 and 7, 16 and 8, 16 and 9, 16 and 10, 16 and 11, 16 and 12, 17 and 2, 17 and 3, 17 and 4, 17 and 5, 17 and 6, 17 and 7, 17 and 8, 17 and 9, 17 and 10, 17 and 11, or 17 and 12. The gRNA core of the PEgRNA can comprise SEQ ID NO: 5857-5859. Exemplary PEgRNAs provided in Table 6 can comprise SEQ ID NOs: 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 106, 107, 108, 109, 111, 116, 117, or 120. Any PEgRNA sequence disclosed in Table 6 may further comprise a 3′ motif at the 3′ end of the extension arm, for example, a hairpin-forming motif or a series of 3, 4 or more U nucleotides. Without being bound by theory, such 3′ motifs are believed to increase PEgRNA stability. Such PEgRNA sequences may alternatively or additionally be adapted for transcription from a DNA template, for example, by including a 5′ terminal G if the spacer of the PEgRNA begins with another nucleotide, by including 6 or 7 U nucleotides at the 3′ end of the extension arm, or both. Exemplary transcription-adapted sequences include SEQ ID NOs: 105, 110, 112, 113, 114, 115, 118, 119, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, and 152. Such plasmid adapted sequences may further comprise a hairpin-forming motif between the PBS and the 3′ terminal U series.

Any of the PEgRNAs of Table 6 can be used in a Prime Editing system further comprising a nick guide RNA (ngRNA). Such ngRNA can comprise a spacer comprising nucleotides 5-20 of any one of SEQ ID NOs: 18-72 and a gRNA core capable of binding to a Cas9 protein. The spacer of the ngRNA can comprise nucleotides 4-20, 3-20, 2-20, or 1-20 of any one of SEQ ID NO: 18-72. The spacer of the ngRNA can comprise any one of SEQ ID NO: 18-72. The gRNA core of the ngRNA can comprise SEQ ID NO: 5857-5859. Exemplary ngRNA provided in Table 6 can comprise any one of SEQ ID NOs: 153-181. Such ngRNA sequences may further comprise a 3′ motif at the 3′ end of the gRNA core, for example, a series of 3, 4 or more U nucleotides. Without being bound by theory, such 3′ motifs are believed to increase ngRNA stability.

Table 7 provides Prime Editing guide RNAs (PEgRNAs), which can be used in Prime Editing systems disclosed herein. Such Prime Editing systems can comprise a Cas9 protein capable of recognizing an NGG, NG, or NNGG PAM sequence (e.g., TGG, TG, or TGGG), and a reverse transcriptase. The Prime Editing systems (e.g., PE3 or PE3b systems) can further comprise a nick guide RNA (ngRNA). Such PEgRNAs and Prime Editing systems can be used, for example, to correct an H1069Q mutation in ATP7B.

The PEgRNAs of Table 7 comprise: (a) a spacer comprising nucleotides 5-20 of SEQ ID NO: 182, (b) a gRNA core capable of binding to a Cas9 protein, and (c) an extension arm comprising: (i) an editing template comprising any one of SEQ ID NOs: 194-198, and (ii) a primer binding site (PBS) comprising any one of SEQ ID NOs: 183-193. The spacer of the PEgRNA can comprise, for example, nucleotides 4-20, 3-20, 2-20, or 1-20 of SEQ ID NO: 182. The spacer of the PEgRNA can comprise SEQ ID NO: 182. The RTT and the PBS can comprise respectively SEQ ID NOs: 194 and 183, 194 and 184, 194 and 185, 194 and 186, 194 and 187, 194 and 188, 194 and 189, 194 and 190, 194 and 191, 194 and 192, 194 and 193, 195 and 183, 195 and 184, 195 and 185, 195 and 186, 195 and 187, 195 and 188, 195 and 189, 195 and 190, 195 and 191, 195 and 192, 195 and 193, 196 and 183, 196 and 184, 196 and 185, 196 and 186, 196 and 187, 196 and 188, 196 and 189, 196 and 190, 196 and 191, 196 and 192, 196 and 193, 197 and 183, 197 and 184, 197 and 185, 197 and 186, 197 and 187, 197 and 188, 197 and 189, 197 and 190, 197 and 191, 197 and 192, 197 and 193, 198 and 183, 198 and 184, 198 and 185, 198 and 186, 198 and 187, 198 and 188, 198 and 189, 198 and 190, 198 and 191, 198 and 192, or 198 and 193. The gRNA core of the PEgRNA can comprise SEQ ID NO: 5857-5859. Exemplary PEgRNAs provided in Table 7 can comprise SEQ ID NOs. 210, 211, 212, 213, 214, 215, 216, 217, 218, 219, 220, 221, 222, 223, 224, 225, 226,227, 228, 229, 230, 231, 232, 233, 234, 235, 236, 237, 238, 239, 240, 241, 242, 243, 245, 247, 248, 250, 251, or 255. Such PEgRNA sequences may further comprise a 3′ motif at the 3′ end of the extension arm, for example, a hairpin-forming motif or a series of 3, 4 or more U nucleotides. Without being bound by theory, such 3′ motifs are believed to increase PEgRNA stability. Such PEgRNA sequences may alternatively or additionally be adapted for transcription from a DNA template, for example, by including a 5′ terminal G if the spacer of the PEgRNA begins with another nucleotide, by including 6 or 7 U nucleotides at the 3′ end of the extension arm, or both. Exemplary transcription-adapted sequences include SEQ ID NOs: 244, 246, 249, 252, 253, 254, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, and 289. Such plasmid adapted sequences may further comprise a hairpin-forming motif between the PBS and the 3′ terminal U series.

Any of the PEgRNAs of Table 7 can be used in a Prime Editing system further comprising a nick guide RNA (ngRNA). Such ngRNA can comprise a spacer comprising nucleotides 5-20 of SEQ ID NO: 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 199, 200, 201, 202, 203, 204, 205, 206, 207, 208, or 209 and a gRNA core capable of binding to a Cas9 protein. The spacer of the ngRNA can comprise nucleotides 4-20, 3-20, 2-20, or 1-20 of SEQ ID NO: 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 199, 200, 201, 202, 203, 204, 205, 206, 207, 208, or 209. The spacer of the ngRNA can comprise SEQ ID NO: 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 199, 200, 201, 202, 203, 204, 205, 206, 207, 208, or 209. The gRNA core of the ngRNA can comprise SEQ ID NO: 5857-5859. Exemplary ngRNA provided in Table 7 can comprise SEQ ID NOs: 153, 154, 155, 156, 157, 158, 159, 160, 161, 162, 163, 164, 165, 166, 167, 168, 169, 170, 171, 172, 173, 174, 175, 176, 177, 178, 179, 180, 181, 290, 291, 292, or 293. Such ngRNA sequences may further comprise a 3′ motif at the 3′ end of the gRNA core, for example, a series of 3, 4 or more U nucleotides. Without being bound by theory, such 3′ motifs are believed to increase ngRNA stability.

Table 8 provides Prime Editing guide RNAs (PEgRNAs), which can be used in Prime Editing systems disclosed herein. Such Prime Editing systems can comprise a Cas9 protein capable of recognizing an NGG, NG, or NNGG PAM sequence (e.g., GGG, GG, GGGG), and a reverse transcriptase. The Prime Editing systems (e.g., PE3 or PE3b systems) can further comprise a nick guide RNA (ngRNA). Such PEgRNAs and Prime Editing systems can be used, for example, to correct an H1069Q mutation in ATP7B.

The PEgRNAs of Table 8 comprise: (a) a spacer comprising nucleotides 5-20 of SEQ ID NO: 294, (b) a gRNA core capable of binding to a Cas9 protein, and (c) an extension arm comprising: (i) an editing template comprising any one of SEQ ID NOs: 306-336, and (ii) a primer binding site (PBS) comprising any one of SEQ ID NOs: 295-305. The spacer of the PEgRNA can comprise, for example, nucleotides 4-20, 3-20, 2-20, or 1-20 of SEQ ID NO: 294. The spacer of the PEgRNA can comprise SEQ ID NO: 294. The RTT and the PBS can comprise respectively SEQ ID NOs: 306 and 295, 306 and 296, 306 and 297, 306 and 298, 306 and 299, 306 and 300, 306 and 301, 306 and 302, 306 and 303, 306 and 304, 306 and 305, 307 and 295, 307 and 296, 307 and 297, 307 and 298, 307 and 299, 307 and 300, 307 and 301, 307 and 302, 307 and 303, 307 and 304, 307 and 305, 308 and 295, 308 and 296, 308 and 297, 308 and 298, 308 and 299, 308 and 300, 308 and 301, 308 and 302, 308 and 303, 308 and 304, 308 and 305, 309 and 295, 309 and 296, 309 and 297, 309 and 298, 309 and 299, 309 and 300, 309 and 301, 309 and 302, 309 and 303, 309 and 304, 309 and 305, 310 and 295, 310 and 296, 310 and 297, 310 and 298, 310 and 299, 310 and 300, 310 and 301, 310 and 302, 310 and 303, 310 and 304, 310 and 305, 311 and 295, 311 and 296, 311 and 297.311 and 298, 311 and 299, 311 and 300, 311 and 301, 311 and 302, 311 and 303, 311 and 304, 311 and 305, 312 and 295, 312 and 296, 312 and 297, 312 and 298, 312 and 299, 312 and 300, 312 and 301, 312 and 302, 312 and 303, 312 and 304, 312 and 305, 313 and 295, 313 and 296, 313 and 297.313 and 298, 313 and 299, 313 and 300, 313 and 301, 313 and 302, 313 and 303, 313 and 304, 313 and 305, 314 and 295, 314 and 296, 314 and 297, 314 and 298, 314 and 299, 314 and 300, 314 and 301, 314 and 302, 314 and 303, 314 and 304, 314 and 305, 315 and 295, 315 and 296, 315 and 297, 315 and 298, 315 and 299, 315 and 300, 315 and 301, 315 and 302, 315 and 303, 315 and 304, 315 and 305, 316 and 295, 316 and 296, 316 and 297, 316 and 298, 316 and 299, 316 and 304, 316 and 301, 316 and 302, 316 and 303, 316 and 304, 316 and 305, 317 and 295, 317 and 296, 317 and 297, 317 and 298, 317 and 299, 317 and 300, 317 and 301, 317 and 302, 317 and 303, 317 and 304, 317 and 305, 318 and 295, 318 and 296, 318 and 297, 318 and 298, 318 and 299, 318 and 300, 318 and 301, 318 and 302, 318 and 303, 318 and 304, 318 and 305, 319 and 295, 319 and 296, 319 and 297, 319 and 298, 319 and 299, 319 and 300, 319 and 301, 319 and 302, 319 and 303, 319 and 304, 319 and 305, 320 and 295, 320 and 296, 320 and 297, 320 and 298, 320 and 299, 320 and 300, 320 and 301, 320 and 302, 320 and 303, 320 and 304, 320 and 305, 321 and 295, 321 and 296, 321 and 297, 321 and 298, 321 and 299, 321 and 300, 321 and 301, 321 and 302, 321 and 303, 321 and 304, 321 and 305, 322 and 295, 322 and 296, 322 and 297, 322 and 298, 322 and 299, 322 and 300, 322 and 301, 322 and 302, 322 and 303, 322 and 304, 322 and 305, 323 and 295, 323 and 296, 323 and 297, 323 and 298, 323 and 299, 323 and 300, 323 and 301, 323 and 302, 323 and 303, 323 and 304, 323 and 305, 324 and 295, 324 and 296, 324 and 297, 324 and 298, 324 and 299, 324 and 300, 324 and 301, 324 and 302, 324 and 303, 324 and 304, 324 and 305, 325 and 295, 325 and 296, 325 and 297, 325 and 298, 325 and 299, 325 and 300, 325 and 301, 325 and 302, 325 and 303, 325 and 304, 325 and 305, 326 and 295, 326 and 296, 326 and 297, 326 and 298, 326 and 299, 326 and 300, 326 and 301, 326 and 302, 326 and 303, 326 and 304, 326 and 305, 327 and 295, 327 and 296, 327 and 297, 327 and 298, 327 and 299, 327 and 300, 327 and 301, 327 and 302, 327 and 303, 327 and 304, 327 and 305, 328 and 295, 328 and 296, 328 and 297, 328 and 298, 328 and 299, 328 and 300, 328 and 301, 328 and 302, 328 and 303, 328 and 304, 328 and 305, 329 and 295, 329 and 296, 329 and 297, 329 and 298, 329 and 299, 329 and 300, 329 and 301, 329 and 302, 329 and 303, 329 and 304, 329 and 305, 330 and 295, 330 and 296, 330 and 297, 330 and 298, 330 and 299, 330 and 300, 330 and 301, 330 and 302, 330 and 303, 330 and 304, 330 and 305, 331 and 295, 331 and 296, 331 and 297, 331 and 298, 331 and 299, 331 and 300, 331 and 301, 331 and 302, 331 and 303, 331 and 304, 331 and 305, 332 and 295, 332 and 296, 332 and 297, 332 and 298, 332 and 299, 332 and 300, 332 and 301, 332 and 302, 332 and 303, 332 and 304, 332 and 305, 333 and 295, 333 and 296, 333 and 297, 333 and 298, 333 and 299, 333 and 300, 333 and 301, 333 and 302, 333 and 303, 333 and 304, 333 and 305, 334 and 295, 334 and 296, 334 and 297, 334 and 298, 334 and 299, 334 and 300, 334 and 301, 334 and 302, 334 and 303, 334 and 304, 334 and 305, 335 and 295, 335 and 296, 335 and 297, 335 and 298, 335 and 299, 335 and 300, 335 and 301, 335 and 302, 335 and 303, 335 and 304, 335 and 305, 336 and 295, 336 and 296, 336 and 297, 336 and 298, 336 and 299, 336 and 300, 336 and 301, 336 and 302, 336 and 303, 336 and 304, or 336 and 305. The gRNA core of the PEgRNA can comprise SEQ ID NOs. any one of 5857-5859. Exemplary PEgRNAs provided in Table 8 can comprise SEQ ID NOs. 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 386, 389, 390, 391, 392, 394, 397, 398, 400, 401, 404, 406, 408, 415, 417, 425, 427, 429, 433, 442, 444, 448, 449, 453, 454, 457, 458, 460, 461, 462, 463, 464, 465, 466, 467, 468, 469, 470, 471, 472, 473, 474, 475, 476, 477, 478, 479, 480, 481, or 482. Such PEgRNA sequences may further comprise a 3′ motif at the 3′ end of the extension arm, for example, a hairpin-forming motif or a series of 3, 4 or more U nucleotides. Without being bound by theory, such 3′ motifs are believed to increase PEgRNA stability. Such PEgRNA sequences may alternatively or additionally be adapted for transcription from a DNA template, for example, by including a 5′ terminal G if the spacer of the PEgRNA begins with another nucleotide, by including 6 or 7 U nucleotides at the 3′ end of the extension arm, or both. Exemplary transcription-adapted sequences include SEQ ID NOs: 385, 387, 388, 393, 395, 396, 399, 402, 403, 405, 407, 409, 410, 411, 412, 413, 414, 416, 418, 419, 420, 421, 422, 423, 424, 426, 428, 430, 431, 432, 434, 435, 436, 437, 438, 439, 440, 441, 443, 445, 446, 447, 450, 451, 452, 455, 456, and 459. Such plasmid adapted sequences may further comprise a hairpin-forming motif between the PBS and the 3′ terminal U series.

Any of the PEgRNAs of Table 8 can be used in a Prime Editing system further comprising a nick guide RNA (ngRNA). Such ngRNA can comprise a spacer comprising nucleotides 5-20 of SEQ ID NO: 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 199, 200, 201, 202, 203, 204, 205, 206, 207, 208, 209, or 337 and a gRNA core capable of binding to a Cas9 protein. The spacer of the ngRNA can comprise nucleotides 4-20, 3-20, 2-20, or 1-20 of SEQ ID NO: 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 199, 200, 201, 202, 203, 204, 205, 206, 207, 208, 209, or 337. The spacer of the ngRNA can comprise SEQ ID NO: 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 199, 200, 201, 202, 203, 204, 205, 206, 207, 208, 209, or 337. The gRNA core of the ngRNA can comprise SEQ ID NO. 5857-5859. Exemplary ngRNA provided in Table 8 can comprise SEQ ID NO: 153, 154, 155, 156, 157, 158, 159, 160, 161, 162, 163, 164, 165, 166, 167, 168, 169, 170, 171, 172, 173, 174, 175, 176, 177, 178, 179, 180, 181, 290, 291, 292, or 293. Such ngRNA sequences may further comprise a 3′ motif at the 3′ end of the gRNA core, for example, a series of 3, 4 or more U nucleotides. Without being bound by theory, such 3′ motifs are believed to increase ngRNA stability.

Table 9 provides Prime Editing guide RNAs (PEgRNAs), which can be used in Prime Editing systems disclosed herein. Such Prime Editing systems can comprise a Cas9 protein capable of recognizing an NGG or NG PAM sequence (e.g., GGG or GG), and a reverse transcriptase. The Prime Editing systems (e.g., PE3 or PE3b systems) can further comprise a nick guide RNA (ngRNA). Such PEgRNAs and Prime Editing systems can be used, for example, to correct an H1069Q mutation in ATP7B.

The PEgRNAs of Table 9 comprise: (a) a spacer comprising nucleotides 5-20 of SEQ ID NO: 483, (b) a gRNA core capable of binding to a Cas9 protein, and (c) an extension arm comprising: (i) an editing template comprising any one of SEQ ID NOs: 495-528, and (ii) a primer binding site (PBS) comprising any one of SEQ ID NOs: 484-494. The spacer of the PEgRNA can comprise, for example, nucleotides 4-20, 3-20, 2-20, or 1-20 of SEQ ID NO: 483. The spacer of the PEgRNA can comprise SEQ ID NO: 483. The RTT and the PBS can comprise respectively SEQ ID NOs: 495 and 484, 495 and 485, 495 and 486, 495 and 487, 495 and 488, 495 and 489, 495 and 490, 495 and 491, 495 and 492, 495 and 493, 495 and 494, 496 and 484, 496 and 485, 496 and 486, 496 and 487, 496 and 488, 496 and 489, 496 and 490, 496 and 491, 496 and 492, 496 and 493, 496 and 494, 497 and 484, 497 and 485, 497 and 486, 497 and 487, 497 and 488, 497 and 489, 497 and 490, 497 and 491, 497 and 492, 497 and 493, 497 and 494, 498 and 484, 498 and 485, 498 and 486, 498 and 487, 498 and 488, 498 and 489, 498 and 490, 498 and 491, 498 and 492, 498 and 493, 498 and 494, 499 and 484, 499 and 485, 499 and 486, 499 and 487, 499 and 488, 499 and 489, 499 and 490, 499 and 491, 499 and 492, 499 and 493, 499 and 494, 500 and 484, 500 and 485, 500 and 486, 500 and 487, 500 and 488, 500 and 489, 500 and 490, 500 and 491, 500 and 492, 500 and 493, 500 and 494, 501 and 484, 501 and 485, 501 and 486, 501 and 487, 501 and 488, 501 and 489, 501 and 490, 501 and 491, 501 and 492, 501 and 493, 501 and 494, 502 and 484, 502 and 485, 502 and 486, 502 and 487, 502 and 488, 502 and 489, 502 and 490, 502 and 491, 502 and 492, 502 and 493, 502 and 494, 503 and 484, 503 and 485, 503 and 486, 503 and 487, 503 and 488, 503 and 489, 503 and 490, 503 and 491, 503 and 492, 503 and 493, 503 and 494, 504 and 484, 504 and 485, 504 and 486, 504 and 487, 504 and 488, 504 and 489, 504 and 490, 504 and 491, 504 and 492, 504 and 493, 504 and 494, 505 and 484, 505 and 485, 505 and 486, 505 and 487, 505 and 488, 505 and 489, 505 and 490, 505 and 491, 505 and 492, 505 and 493, 505 and 494, 506 and 484, 506 and 485, 506 and 486, 506 and 487, 506 and 488, 506 and 489, 506 and 490, 506 and 491, 506 and 492, 506 and 493, 506 and 494, 507 and 484, 507 and 485, 507 and 486, 507 and 487, 507 and 488, 507 and 489, 507 and 490, 507 and 491, 507 and 492, 507 and 493, 507 and 494, 508 and 484, 508 and 485, 508 and 486, 508 and 487, 508 and 488, 508 and 489, 508 and 490, 508 and 491, 508 and 492, 508 and 493, 508 and 494, 509 and 484, 509 and 485, 509 and 486, 509 and 487, 509 and 488, 509 and 489, 509 and 490, 509 and 491, 509 and 492, 509 and 493, 509 and 494, 510 and 484, 510 and 485, 510 and 486, 510 and 487, 510 and 488, 510 and 489, 510 and 490, 510 and 491, 510 and 492, 510 and 493, 510 and 494, 511 and 484, 511 and 485, 511 and 486, 511 and 487, 511 and 488, 511 and 489, 511 and 490, 511 and 491, 511 and 492, 511 and 493, 511 and 494, 512 and 484, 512 and 485, 512 and 486, 512 and 487, 512 and 488, 512 and 489, 512 and 490, 512 and 491, 512 and 492, 512 and 493, 512 and 494, 513 and 484, 513 and 485, 513 and 486, 513 and 487, 513 and 488, 513 and 489, 513 and 490, 513 and 491, 513 and 492, 513 and 493, 513 and 494, 514 and 484, 514 and 485, 514 and 486, 514 and 487, 514 and 488, 514 and 489, 514 and 490, 514 and 491, 514 and 492, 514 and 493, 514 and 494, 515 and 484, 515 and 485, 515 and 486, 515 and 487, 515 and 488, 515 and 489, 515 and 490, 515 and 491, 515 and 492, 515 and 493, 515 and 494, 516 and 484, 516 and 485, 516 and 486, 516 and 487, 516 and 488, 516 and 489, 516 and 490, 516 and 491, 516 and 492, 516 and 493, 516 and 494, 517 and 484, 517 and 485, 517 and 486, 517 and 487, 517 and 488, 517 and 489, 517 and 490, 517 and 491, 517 and 492, 517 and 493, 517 and 494, 518 and 484, 518 and 485, 518 and 486, 518 and 487, 518 and 488, 518 and 489, 518 and 490, 518 and 491, 518 and 492, 518 and 493, 518 and 494, 519 and 484, 519 and 485, 519 and 486, 519 and 487, 519 and 488, 519 and 489, 519 and 490, 519 and 491, 519 and 492, 519 and 493, 519 and 494, 520 and 484, 520 and 485, 520 and 486, 520 and 487, 520 and 488, 520 and 489, 520 and 490, 520 and 491, 520 and 492, 520 and 493, 520 and 494, 521 and 484, 521 and 485, 521 and 486, 521 and 487, 521 and 488, 521 and 489, 521 and 490, 521 and 491, 521 and 492, 521 and 493, 521 and 494, 522 and 484, 522 and 485, 522 and 486, 522 and 487, 522 and 488, 522 and 489, 522 and 490, 522 and 491, 522 and 492, 522 and 493, 522 and 494, 523 and 484, 523 and 485, 523 and 486, 523 and 487, 523 and 488, 523 and 489, 523 and 490, 523 and 491, 523 and 492, 523 and 493, 523 and 494, 524 and 484, 524 and 485, 524 and 486, 524 and 487, 524 and 488, 524 and 489, 524 and 490, 524 and 491, 524 and 492, 524 and 493, 524 and 494, 525 and 484, 525 and 485, 525 and 486, 525 and 487, 525 and 488, 525 and 489, 525 and 490, 525 and 491, 525 and 492, 525 and 493, 525 and 494, 526 and 484, 526 and 485, 526 and 486, 526 and 487, 526 and 488, 526 and 489, 526 and 490, 526 and 491, 526 and 492, 526 and 493, 526 and 494, 527 and 484, 527 and 485, 527 and 486, 527 and 487, 527 and 488, 527 and 489, 527 and 490, 527 and 491, 527 and 492, 527 and 493, 527 and 494, 528 and 484, 528 and 485, 528 and 486, 528 and 487, 528 and 488, 528 and 489, 528 and 490, 528 and 491, 528 and 492, 528 and 493, or 528 and 494. The gRNA core of the PEgRNA can comprise SEQ ID NO. 5857-5859. Exemplary PEgRNAs provided in Table 9 can comprise SEQ ID NO. 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 574, 575, 577, 578, 580, 581, 582, 583, 584, 585, 586, 588, 592, 593, 594, 597, 598, 600, 601, 608, 609, 612, 620, 621, 627, 628, 631, 639, 640, 644, 647, 649, 650, 654, 655, 656, 657, 658, 659, 660, 661, 662, 663, 664, 665, 666, 667, 668, 669, 670, 671, 672, 673, 674, 675, 676, 677, 678, 679, or 680. Such PEgRNA sequences may further comprise a 3′ motif at the 3′ end of the extension arm, for example, a hairpin-forming motif or a series of 3, 4 or more U nucleotides. Without being bound by theory, such 3′ motifs are believed to increase PEgRNA stability. Such PEgRNA sequences may alternatively or additionally be adapted for transcription from a DNA template, for example, by including a 5′ terminal G if the spacer of the PEgRNA begins with another nucleotide, by including 6 or 7 U nucleotides at the 3′ end of the extension arm, or both. Exemplary transcription-adapted sequences include SEQ ID NOs: 573, 576, 579, 587, 589, 590, 591, 595, 596, 599, 602, 603, 604, 605, 606, 607, 610, 611, 613, 614, 615, 616, 617,618, 619, 622, 623, 624, 625, 626, 629, 630, 632, 633, 634, 635, 636, 637, 638, 641, 642, 643, 645, 646, 648, 651, 652, and 653. Such plasm id adapted sequences may further comprise a hairpin-forming motif between the PBS and the 3′ terminal U series.

Any of the PEgRNAs of Table 9 can be used in a Prime Editing system further comprising a nick guide RNA (ngRNA). Such ngRNA can comprise a spacer comprising nucleotides 5-20 of SEQ ID NO: 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 199, 200, 203, or 529 and a gRNA core capable of binding to a Cas9 protein. The spacer of the ngRNA can comprise nucleotides 4-20, 3-20, 2-20, or 1-20 of SEQ ID NO: 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 199, 200, 203, or 529. The spacer of the ngRNA can comprise SEQ ID NO: 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 199, 200, 203, or 529. The gRNA core of the ngRNA can comprise SEQ ID NO. 5857-5859. Exemplary ngRNA provided in Table 9 can comprise SEQ ID NO: 153, 154, 155, 156, 157, 158, 159, 160, 161, 162, 163, 164, 165, 166, 167, 168, 169, 170, 171, 172, 173, 174, 175, 176, 177, 178, 179, 180, 181, 290, 291, 292, 293, or 681. Such ngRNA sequences may further comprise a 3′ motif at the 3′ end of the gRNA core, for example, a series of 3, 4 or more U nucleotides. Without being bound by theory, such 3′ motifs are believed to increase ngRNA stability.

Table 10 provides Prime Editing guide RNAs (PEgRNAs), which can be used in Prime Editing systems disclosed herein. Such Prime Editing systems can comprise a Cas9 protein capable of recognizing an NOG or NO PAM sequence (e.g., CGG or CG), and a reverse transcriptase. The Prime Editing systems (e.g., PE3 or PE3b systems) can further comprise a nick guide RNA (ngRNA). Such PEgRNAs and Prime Editing systems can be used, for example, to correct an 11069Q mutation in ATP7B.

The PEgRNAs of Table 10 comprise: (a) a spacer comprising nucleotides 5-20 of SEQ ID NO: 682, (b) a gRNA core capable of binding to a Cas9 protein, and (c) an extension arm comprising: (i) an editing template comprising any one of SEQ ID NOs: 694-735, and (ii) a primer binding site (PBS) comprising any one of SEQ ID NOs: 683-693. The spacer of the PEgRNA can comprise, for example, nucleotides 4-20, 3-20, 2-20, or 1-20 of SEQ ID NO: 682. The spacer of the PEgRNA can comprise SEQ ID NO: 682. The RTT and the PBS can comprise respectively SEQ ID NOs: 694 and 683, 694 and 684, 694 and 685, 694 and 686, 694 and 687, 694 and 688, 694 and 689, 694 and 690, 694 and 691, 694 and 692, 694 and 693, 695 and 683, 695 and 684, 695 and 685, 695 and 686, 695 and 687, 695 and 688, 695 and 689, 695 and 690, 695 and 691, 695 and 692, 695 and 693, 696 and 683, 696 and 684, 696 and 685, 696 and 686, 696 and 687, 696 and 688, 696 and 689, 696 and 690, 696 and 691, 696 and 692, 696 and 693, 697 and 683, 697 and 684, 697 and 685, 697 and 686, 697 and 687, 697 and 688, 697 and 689, 697 and 690, 697 and 691, 697 and 692, 697 and 693, 698 and 683, 698 and 684, 698 and 685, 698 and 686, 698 and 687, 698 and 688, 698 and 689, 698 and 690, 698 and 691, 698 and 692, 698 and 693, 699 and 683, 699 and 684, 699 and 685, 699 and 686, 699 and 687, 699 and 688, 699 and 689, 699 and 690, 699 and 691, 699 and 692, 699 and 693, 700 and 683, 700 and 684, 700 and 685, 700 and 686, 700 and 687, 700 and 688, 700 and 689, 700 and 690, 700 and 691, 700 and 692, 700 and 693, 701 and 683, 701 and 684, 701 and 685, 701 and 686, 701 and 687, 701 and 688, 701 and 689, 701 and 690, 701 and 691, 701 and 692, 701 and 693, 702 and 683, 702 and 684, 702 and 685, 702 and 686, 702 and 687, 702 and 688, 702 and 689, 702 and 690, 702 and 691, 702 and 692, 702 and 693, 703 and 683, 703 and 684, 703 and 685, 703 and 686, 703 and 687, 703 and 688, 703 and 689, 703 and 690, 703 and 691, 703 and 692, 703 and 693, 704 and 683, 704 and 684, 704 and 685, 704 and 686, 704 and 687, 704 and 688, 704 and 689, 704 and 690, 704 and 691, 704 and 692, 704 and 693, 705 and 683, 705 and 684, 705 and 685, 705 and 686, 705 and 687, 705 and 688, 705 and 689, 705 and 690, 705 and 691, 705 and 692, 705 and 693, 706 and 683, 706 and 684, 706 and 685, 706 and 686, 706 and 687, 706 and 688, 706 and 689, 706 and 690, 706 and 691, 706 and 692, 706 and 693, 707 and 683, 707 and 684, 707 and 685, 707 and 686, 707 and 687, 707 and 688, 707 and 689, 707 and 690, 707 and 691, 707 and 692, 707 and 693, 708 and 683, 708 and 684, 708 and 685, 708 and 686, 708 and 687, 708 and 688, 708 and 689, 708 and 690, 708 and 691, 708 and 692, 708 and 693, 709 and 683, 709 and 684, 709 and 685, 709 and 686, 709 and 687, 709 and 688, 709 and 689, 709 and 690, 709 and 691, 709 and 692, 709 and 693, 710 and 683, 710 and 684, 710 and 685, 710 and 686, 710 and 687, 710 and 688, 710 and 689, 710 and 690, 710 and 691, 710 and 692, 710 and 693, 711 and 683, 711 and 684, 711 and 685, 711 and 686, 711 and 687, 711 and 688, 711 and 689, 711 and 690, 711 and 691, 711 and 692, 711 and 693, 712 and 683, 712 and 684, 712 and 685, 712 and 686, 712 and 687, 712 and 688, 712 and 689, 712 and 690, 712 and 691, 712 and 692, 712 and 693, 713 and 683, 713 and 684, 713 and 685, 713 and 686, 713 and 687, 713 and 688, 713 and 689, 713 and 690, 713 and 691, 713 and 692, 713 and 693, 714 and 683, 714 and 684, 714 and 685, 714 and 686, 714 and 687, 714 and 688, 714 and 689, 714 and 690, 714 and 691, 714 and 692, 714 and 693, 715 and 683, 715 and 684, 715 and 685, 715 and 686, 715 and 687, 715 and 688, 715 and 689, 715 and 690, 715 and 691, 715 and 692, 715 and 693, 716 and 683, 716 and 684, 716 and 685, 716 and 686, 716 and 687, 716 and 688, 716 and 689, 716 and 690, 716 and 691, 716 and 692, 716 and 693, 717 and 683, 717 and 684, 717 and 685, 717 and 686, 717 and 687, 717 and 688, 717 and 689, 717 and 690, 717 and 691, 717 and 692, 717 and 693, 718 and 683, 718 and 684, 718 and 685, 718 and 686, 718 and 687, 718 and 688, 718 and 689, 718 and 690, 718 and 691, 718 and 692, 718 and 693, 719 and 683, 719 and 684, 719 and 685, 719 and 686, 719 and 687, 719 and 688, 719 and 689, 719 and 690, 719 and 691, 719 and 692, 719 and 693, 720 and 683, 720 and 684, 720 and 685, 720 and 686, 720 and 687, 720 and 688, 720 and 689, 720 and 690, 720 and 691, 720 and 692, 720 and 693, 721 and 683, 721 and 684, 721 and 685, 721 and 686, 721 and 687, 721 and 688, 721 and 689, 721 and 690, 721 and 691, 721 and 692, 721 and 693, 722 and 683, 722 and 684, 722 and 685, 722 and 686, 722 and 687, 722 and 688, 722 and 689, 722 and 690, 722 and 691, 722 and 692, 722 and 693, 723 and 683, 723 and 684, 723 and 685, 723 and 686, 723 and 687, 723 and 688, 723 and 689, 723 and 690, 723 and 691, 723 and 692, 723 and 693, 724 and 683, 724 and 684, 724 and 685, 724 and 686, 724 and 687, 724 and 688, 724 and 689, 724 and 690, 724 and 691, 724 and 692, 724 and 693, 725 and 683, 725 and 684, 725 and 685, 725 and 686, 725 and 687, 725 and 688, 725 and 689, 725 and 690, 725 and 691, 725 and 692, 725 and 693, 726 and 683, 726 and 684, 726 and 685, 726 and 686, 726 and 687, 726 and 688, 726 and 689, 726 and 690, 726 and 691, 726 and 692, 726 and 693, 727 and 683, 727 and 684, 727 and 685, 727 and 686, 727 and 687, 727 and 688, 727 and 689, 727 and 690, 727 and 691, 727 and 692, 727 and 693, 728 and 683, 728 and 684, 728 and 685, 728 and 686, 728 and 687, 728 and 688, 728 and 689, 728 and 690, 728 and 691, 728 and 692, 728 and 693, 729 and 683, 729 and 684, 729 and 685, 729 and 686, 729 and 687, 729 and 688, 729 and 689, 729 and 690, 729 and 691, 729 and 692, 729 and 693, 730 and 683, 730 and 684, 730 and 685, 730 and 686, 730 and 687, 730 and 688, 730 and 689, 730 and 690, 730 and 691, 730 and 692, 730 and 693, 731 and 683, 731 and 684, 731 and 685, 731 and 686, 731 and 687, 731 and 688, 731 and 689, 731 and 690, 731 and 691, 731 and 692, 731 and 693, 732 and 683, 732 and 684, 732 and 685, 732 and 686, 732 and 687, 732 and 688, 732 and 689, 732 and 690, 732 and 691, 732 and 692, 732 and 693, 733 and 683, 733 and 684, 733 and 685, 733 and 686, 733 and 687, 733 and 688, 733 and 689, 733 and 690, 733 and 691, 733 and 692, 733 and 693, 734 and 683, 734 and 684, 734 and 685, 734 and 686, 734 and 687, 734 and 688, 734 and 689, 734 and 690, 734 and 691, 734 and 692, 734 and 693, 735 and 683, 735 and 684, 735 and 685, 735 and 686, 735 and 687, 735 and 688, 735 and 689, 735 and 690, 735 and 691, 735 and 692, or 735 and 693, The gRNA core of the PEgRNA can comprise SEQ ID NO. 5857-5859. Exemplary PEgRNAs provided in Table 10 can comprise SEQ ID NO. 741, 742, 743, 744, 745, 746, 747, 748, 749, 750, 751, 752, 753, 754, 755, 756, 757, 758, 759, 760, 761, 762, 763, 764, 765, 766, 767, 768, 769, 770, 771, 772, 773, 774, 775, 776, 777, 778, 779, 780, 781, 782, 783, 784, 785, 786, 787, 788, 789, 790, 791, 792, 793, 794, 795, 796, 797, 798, 799, 800, 801, 802, 803, 804, 805, 806, 807, 808, 809, 810, 811, 812, 813, 814, 815, 816, 817, 818, 819, 820, 821, 822, 823, 824, 825, 826, 827, 828, 829, 830, 831, 832, 833, 834, 835, 836, 837, 838, 839, 840, 841, 842, 843, 844, 845, 846, 847, 848, 849, 850, 851, 852, 853, 854, 855, 856, 857, 858, 859, 860, 861, 862, 863, 865, 866, 869, 870, 871, 872, 873, 874, 878, 879, 880, 881, 882, 883, 885, 887, 888, 890, 893, 894, 895, 896, 897, 898, 900, 902, 905, 906, 908, 909, 910, 911, 912, 914, 916, 918, 920, 922, 923, 924, 925, 926, 927, 928, 929, 930, 931, 932, 933, 934, 935, 936, 937, 938, 939, 959, 960, 961, 962, 963, 965, 966, 967, 970, 971, 977, 979, 983, 987, 989, 992, 997, 1000, 1018, 1019, 1020, 1021, 1022, 1023, 1024, 1025, 1037, 1041, 1043, 1044, 1045, 1046, 1050, 1057, 1068, 1074, 1076, 1077, 1078, 1079, 1080, 1081, 1082, 1084, 1085, 1087, 1101, 1103, 1104, 1105, 1106, 1107, 1108, 1109, 1110, 1111, 1112, 1113, 1114, 1115, 1116, 1124, 1139, 1140, 1141, 1144, 1147, 1151, 1155, 1159, 1160, 1163, 1166, 1167, 1168, 1169, 1171, 1172, 1185, 1187, 1190, 1191, 1197, 1199, 1203, 1208, 1209, 1210, 1218, 1219, 1220, 1221, 1222, 1223, 1229, 1230, 1233, 1235, 1236, 1237, 1240, 1244, 1248, 1261, 1263, 1264, 1265, 1266, 1267, 1268, 1269, 1270, 1272, 1273, 1278, 1282, 1285, 1289, 1292, 1294, 1297, 1299, 1305, 1309, 1310, 1311, 1313, 1314, 1315, 1316, 1317, 1318, 1319, 1329, 1333, 1334, 1338, 1340, 1342, 1344, 1345, 1346, 1347, 1348, 1353, 1355, 1357, 1362, 1363, 1364, 1368, 1369, 1370, 1376, 1383, 1388, 1395, 1396, 1413, 1414, 1415, or 1416. Such PEgRNA sequences may further comprise a 3′ motif at the 3′ end of the extension arm, for example, a hairpin-forming motif or a series of 3, 4 or more U nucleotides. Without being bound by theory, such 3′ motifs are believed to increase PEgRNA stability. Such PEgRNA sequences may alternatively or additionally be adapted for transcription from a DNA template, for example, by including a 5′ terminal G if the spacer of the PEgRNA begins with another nucleotide, by including 6 or 7 U nucleotides at the 3′ end of the extension arm, or both. Exemplary transcription-adapted sequences include SEQ ID NOs: 864, 867, 868, 875, 876, 877, 884, 886, 889, 891, 892, 899, 901, 903, 904, 907, 913, 915, 917, 919, 921, 940, 941, 942, 943, 944, 945, 946, 947, 948,949, 950, 951, 952, 953, 954, 955, 956, 957, 958, 964, 968, 969, 972, 973, 974, 975, 976, 978, 980, 981, 982, 984, 985, 986, 988, 990, 991, 993, 994, 995, 996, 998, 999, 1001, 1002, 1003, 1004, 1005, 1006, 1007, 1008, 1009, 1010, 1011, 1012, 1013, 1014, 1015, 1016, 1017, 1026, 1027, 1028, 1029, 1030, 1031, 1032, 1033, 1034, 1035, 1036, 1038, 1039, 1040, 1042, 1047, 1048, 1049, 1051, 1052, 1053, 1054, 1055, 1056, 1058, 1059, 1060, 1061, 1062, 1063, 1064, 1065, 1066, 1067, 1069, 1070, 1071, 1072, 1073, 1075, 1083, 1086, 1088, 1089, 1090, 1091, 1092, 1093, 1094, 1095, 1096, 1097, 1098, 1099, 1100, 1102, 1117, 1118, 1119, 1120, 1121, 1122, 1123, 1125, 1126, 1127, 1128, 1129, 1130, 1131, 1132, 1133, 1134, 1135, 1136, 1137, 1138, 1142, 1143, 1145, 1146, 1148, 1149, 1150, 1152, 1153, 1154, 1156, 1157, 1158, 1161, 1162, 1164, 1165, 1170, 1173, 1174, 1175, 1176, 1177, 1178, 1179, 1180, 1181, 1182, 1183, 1184, 1186, 1188, 1189, 1192, 1193, 1194, 1195, 1196, 1198, 1200, 1201, 1202, 1204, 1205, 1206, 1207, 1211, 1212, 1213, 1214, 1215, 1216, 1217, 1224, 1225, 1226, 1227, 1228, 1231, 1232, 1234, 1238, 1239, 1241, 1242, 1243, 1245, 1246, 1247, 1249, 1250, 1251, 1252, 1253, 1254, 1255, 1256, 1257, 1258, 1259, 1260, 1262, 1271, 1274, 1275, 1276, 1277, 1279, 1280, 1281, 1283, 1284, 1286, 1287, 1288, 1290, 1291, 1293, 1295, 1296, 1298, 1300, 1301, 1302, 1303, 1304, 1306, 1307, 1308, 1312, 1320, 1321, 1322, 1323, 1324, 1325, 1326, 1327, 1328, 1330, 1331, 1332, 1335, 1336, 1337, 1339, 1341, 1343, 1349, 1350, 1351, 1352, 1354, 1356, 1358, 1359, 1360, 1361, 1365, 1366, 1367, 1371, 1372, 1373, 1374, 1375, 1377, 1378, 1379, 1380, 1381, 1382, 1384, 1385, 1386, 1387, 1389, 1390, 1391, 1392, 1393, 1394, 1397, 1398, 1399, 1400, 1401, 1402, 1403, 1404, 1405, 1406, 1407, 1408, 1409, 1410, 1411, 1412, 1417, 1418, 1419, 1420, 1421, 1422, 1423, 1424, 1425, 1426, 1427, 1428, 1429, 1430, 1431, 1432, 1433, 1434, 1435, 1436, 1437, 1438, 1439, 1440, 1441, 1442, 1443, 1444, 1445, 1446, 1447, 1448, 1449, 1450, 1451, 1452, 1453, 1454, 1455, 1456, 1457, 1458, 1459, 1460, 1461, 1462, 1463, 1464, 1465, 1466, 1467, 1468, 1469, 1470, 1471, 1472, 1473, 1474, 1475, 1476, 1477, 1478, 1479, 1480, 1481, 1482, 1483, 1484, 1485, 1486, 1487, 1488, 1489, 1490, 1491, 1492, 1493, 1494, 1495, 1496, 1497, 1498, 1499, and 1500. Such plasmid adapted sequences may further comprise a hairpin-forming motif between the PBS and the 3′ terminal U series.

Any of the PEgRNAs of Table 10 can be used in a Prime Editing system further comprising a nick guide RNA (ngRNA). Such ngRNA can comprise a spacer comprising nucleotides 5-20 of SEQ ID NO: 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 199, 200, 203, 529, 736, 737, 738, 739, or 740 and a gRNA core capable of binding to a Cas9 protein. The spacer of the ngRNA can comprise nucleotides 4-20, 3-20, 2-20, or 1-20 of SEQ ID NO: 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 199, 200, 203, 529, 736, 737, 738, 739, or 740. The spacer of the ngRNA can comprise SEQ ID NO: 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 199, 200, 203, 529, 736, 737, 738, 739, or 740. The gRNA core of the ngRNA can comprise SEQ ID NO. 5857-5859. Exemplary ngRNA provided in Table 10 can comprise SEQ ID NO: 153, 154, 155, 156, 157, 158, 159, 160, 161, 162, 163, 164, 165, 166, 167, 168, 169, 170, 171, 172, 173, 174, 175, 176, 177, 178, 179, 180, 181, 290, 291, 292, 293, 681, 1501, 1502, 1503, or 1504. Such ngRNA sequences may further comprise a 3′ motif at the 3′ end of the gRNA core, for example, a series of 3, 4 or more U nucleotides. Without being bound by theory, such 3′ motifs are believed to increase ngRNA stability.

Table 11 provides Prime Editing guide RNAs (PEgRNAs), which can be used in Prime Editing systems disclosed herein. Such Prime Editing systems can comprise a Cas9 protein capable of recognizing an NGG or NG PAM sequence (e.g., AGG or AG), and a reverse transcriptase. The Prime Editing systems (e.g., PE3 or PE3b systems) can further comprise a nick guide RNA (ngRNA). Such PEgRNAs and Prime Editing systems can be used, for example, to correct an 1H1069Q mutation in ATP7B.

The PEgRNAs of Table 11 comprise: (a) a spacer comprising nucleotides 5-20 of SEQ ID NO: 1505, (b) a gRNA core capable of binding to a Cas9 protein, and (c) an extension arm comprising: (i) an editing template comprising any one of SEQ ID NOs: 1517-1546, and (ii) a primer binding site (PBS) comprising any one of SEQ ID NOs: 1506-1516. The spacer of the PEgRNA can comprise, for example, nucleotides 4-20, 3-20, 2-20, or 1-20 of SEQ ID NO: 1505. The spacer of the PEgRNA can comprise SEQ ID NO: 1505. The RTT and the PBS can comprise respectively SEQ ID NOs: 1517 and 1506, 1517 and 1507, 1517 and 1508, 1517 and 1509, 1517 and 1510, 1517 and 1511, 1517 and 1512, 1517 and 1513, 1517 and 1514, 1517 and 1515, 1517 and 1516, 1518 and 1506, 1518 and 1507, 1518 and 1508, 1518 and 1509, 1518 and 1510, 1518 and 1511, 1518 and 1512, 1518 and 1513, 1518 and 1514, 1518 and 1515, 1518 and 1516, 1519 and 1506, 1519 and 1507, 1519 and 1508, 1519 and 1509, 1519 and 1510, 1519 and 1511, 1519 and 1512, 1519 and 1513, 1519 and 1514, 1519 and 1515, 1519 and 1516, 1520 and 1506, 1520 and 1507, 1520 and 1508, 1520 and 1509, 1520 and 1510, 1520 and 1511, 1520 and 1512, 1520 and 1513, 1520 and 1514, 1520 and 1515, 1520 and 1516, 1521 and 1506, 1521 and 1507, 1521 and 1508, 1521 and 1509, 1521 and 1510, 1521 and 1511, 1521 and 1512, 1521 and 1513, 1521 and 1514, 1521 and 1515, 1521 and 1516, 1522 and 1506, 1522 and 1507, 1522 and 1508, 1522 and 1509, 1522 and 1510, 1522 and 1511, 1522 and 1512, 1522 and 1513, 1522 and 1514, 1522 and 1515, 1522 and 1516, 1523 and 1506, 1523 and 1507, 1523 and 1508, 1523 and 1509, 1523 and 1510, 1523 and 1511, 1523 and 1512, 1523 and 1513, 1523 and 1514, 1523 and 1515, 1523 and 1516, 1524 and 1506, 1524 and 1507, 1524 and 1508, 1524 and 1509, 1524 and 1510, 1524 and 1511, 1524 and 1512, 1524 and 1513, 1524 and 1514, 1524 and 1515, 1524 and 1516, 1525 and 1506, 1525 and 1507, 1525 and 1508, 1525 and 1509, 1525 and 1510, 1525 and 1511, 1525 and 1512, 1525 and 1513, 1525 and 1514, 1525 and 1515, 1525 and 1516, 1526 and 1506, 1526 and 1507, 1526 and 1508, 1526 and 1509, 1526 and 1510, 1526 and 1511, 1526 and 1512, 1526 and 1513, 1526 and 1514, 1526 and 1515, 1526 and 1516, 1527 and 1506, 1527 and 1507, 1527 and 1508, 1527 and 1509, 1527 and 1510, 1527 and 1511, 1527 and 1512, 1527 and 1513, 1527 and 1514, 1527 and 1515, 1527 and 1516, 1528 and 1506, 1528 and 1507, 1528 and 1508, 1528 and 1509, 1528 and 1510, 1528 and 1511, 1528 and 1512, 1528 and 1513, 1528 and 1514, 1528 and 1515, 1528 and 1516, 1529 and 1506, 1529 and 1507, 1529 and 1508, 1529 and 1509, 1529 and 1510, 1529 and 1511, 1529 and 1512, 1529 and 1513, 1529 and 1514, 1529 and 1515, 1529 and 1516, 1530 and 1506, 1530 and 1507, 1530 and 1508, 1530 and 1509, 1530 and 1510, 1530 and 1511, 1530 and 1512, 1530 and 1513, 1530 and 1514, 1530 and 1515, 1530 and 1516, 1531 and 1506, 1531 and 1507, 1531 and 1508, 1531 and 1509, 1531 and 1510, 1531 and 1511, 1531 and 1512, 1531 and 1513, 1531 and 1514, 1531 and 1515, 1531 and 1516, 1532 and 1506, 1532 and 1507, 1532 and 1508, 1532 and 1509, 1532 and 1510, 1532 and 1511, 1532 and 1512, 1532 and 1513, 1532 and 1514, 1532 and 1515, 1532 and 1516, 1533 and 1506, 1533 and 1507, 1533 and 1508, 1533 and 1509, 1533 and 1510, 1533 and 1511, 1533 and 1512, 1533 and 1513, 1533 and 1514, 1533 and 1515, 1533 and 1516, 1534 and 1506, 1534 and 1507, 1534 and 1508, 1534 and 1509, 1534 and 1510, 1534 and 1511, 1534 and 1512, 1534 and 1513, 1534 and 1514, 1534 and 1515, 1534 and 1516, 1535 and 1506, 1535 and 1507, 1535 and 1508, 1535 and 1509, 1535 and 1510, 1535 and 1511, 1535 and 1512, 1535 and 1513, 1535 and 1514, 1535 and 1515, 1535 and 1516, 1536 and 1506, 1536 and 1507, 1536 and 1508, 1536 and 1509, 1536 and 1510, 1536 and 1511, 1536 and 1512, 1536 and 1513, 1536 and 1514, 1536 and 1515, 1536 and 1516, 1537 and 1506, 1537 and 1507, 1537 and 1508, 1537 and 1509, 1537 and 1510, 1537 and 1511, 1537 and 1512, 1537 and 1513, 1537 and 1514, 1537 and 1515, 1537 and 1516, 1538 and 1506, 1538 and 1507, 1538 and 1508, 1538 and 1509, 1538 and 1510, 1538 and 1511, 1538 and 1512, 1538 and 1513, 1538 and 1514, 1538 and 1515, 1538 and 1516, 1539 and 1506, 1539 and 1507, 1539 and 1508, 1539 and 1509, 1539 and 1510, 1539 and 1511, 1539 and 1512, 1539 and 1513, 1539 and 1514, 1539 and 1515, 1539 and 1516, 1540 and 1506, 1540 and 1507, 1540 and 1508, 1540 and 1509, 1540 and 1510, 1540 and 1511, 1540 and 1512, 1540 and 1513, 1540 and 1514, 1540 and 1515, 1540 and 1516, 1541 and 1506, 1541 and 1507, 1541 and 1508, 1541 and 1509, 1541 and 1510, 1541 and 1511, 1541 and 1512, 1541 and 1513, 1541 and 1514, 1541 and 1515, 1541 and 1516, 1542 and 1506, 1542 and 1507, 1542 and 1508, 1542 and 1509, 1542 and 1510, 1542 and 1511, 1542 and 1512, 1542 and 1513, 1542 and 1514, 1542 and 1515, 1542 and 1516, 1543 and 1506, 1543 and 1507, 1543 and 1508, 1543 and 1509, 1543 and 1510, 1543 and 1511, 1543 and 1512, 1543 and 1513, 1543 and 1514, 1543 and 1515, 1543 and 1516, 1544 and 1506, 1544 and 1507, 1544 and 1508, 1544 and 1509, 1544 and 1510, 1544 and 1511, 1544 and 1512, 1544 and 1513, 1544 and 1514, 1544 and 1515, 1544 and 1516, 1545 and 1506, 1545 and 1507, 1545 and 1508, 1545 and 1509, 1545 and 1510, 1545 and 1511, 1545 and 1512, 1545 and 1513, 1545 and 1514, 1545 and 1515, 1545 and 1516, 1546 and 1506, 1546 and 1507, 1546 and 1508, 1546 and 1509, 1546 and 1510, 1546 and 1511, 1546 and 1512, 1546 and 1513, 1546 and 1514, 1546 and 1515, or 1546 and 1516. The gRNA core of the PEgRNA can comprise SEQ ID NO. 5857-5859. Exemplary PEgRNAs provided in Table 11 can comprise SEQ ID NO. 1547, 1548, 1549, 1550, 1551, 1552, 1553, 1554, 1555, 1556, 1557, 1558, 1559, 1560, 1561, 1562, 1563, 1564, 1565, 1566, 1567, 1568, 1569, 1570, 1571, 1572, 1573, 1574, 1575, 1576, 1577, 1578, 1579, 1580, 1581, 1582, 1583, 1584, 1585, 1586, 1587, 1588, 1589, 1590, 1591, 1592, 1593, 1594, 1595, 1596, 1598, 1600, 1601, 1602, 1604, 1606, 1607, 1608, 1609, 1610, 1614, 1615, 1616, 1619, 1620, 1621, 1622, 1623, 1624, 1626, 1628, 1632, 1633, 1634, 1635, 1637, 1640, 1643, 1645, 1646, 1647, 1648, 1649, 1650, 1651, 1653, 1654, 1663, 1664, 1668, 1671, 1678, 1679, 1684, 1686, 1687, 1688, 1691, 1694, 1696, 1697, 1699, 1702, 1708, 1721, 1725, 1728, 1729, 1730, 1731, 1732, 1733, 1737, 1738, 1741, 1742, 1743, 1744, 1745, 1756, 1760, 1761, 1766, 1769, 1770, 1771, 1773, 1777, 1785, 1786, 1788, 1789, 1792, 1796, 1798, 1801, 1803, 1804, 1805, 1806, 1807, 1810, 1813, 1815, 1819, 1824, 1825, 1826, 1830, 1832, 1833, 1835, 1837, 1840, 1841, 1842, 1845, 1846, 1847, 1848, 1850, 1852, 1855, 1856, 1857, 1858, 1859, 1860, 1861, 1862, 1863, 1864, 1865, 1866, 1868, 1870, 1872, 1874, 1876, 1877, 1880, 1882, 1883, 1884, 1888, 1890, 1892, 1893, 1895, 1896, 1902, 1905, 1907, 1908, 1909, 1910, 1911, 1912, 1915, 1918, 1919, 1920, 1921, 1923, 1924, 1928, 1930, 1933, 1935, 1940, 1941, 1943, 1945, 1949, 1954, 1958, 1966, or 1967. Such PEgRNA sequences may further comprise a 3′ motif at the 3′ end of the extension arm, for example, a hairpin-forming motif or a series of 3, 4 or more U nucleotides. Without being bound by theory, such 3′ motifs are believed to increase PEgRNA stability. Such PEgRNA sequences ma alternatively or additionally be adapted for transcription from a DNA template, for example, by including a 5′ terminal G if the spacer of the PEgRNA begins with another nucleotide, by including 6 or 7 U nucleotides at the 3′ end of the extension arm, or both. Exemplary transcription-adapted sequences include SEQ ID NOs: 1597, 1599, 1603, 1605, 1611, 1612, 1613, 1617, 1618, 1625, 1627, 1629, 1630, 1631, 1636, 1638, 1639, 1641, 1642, 1644, 1652, 1655, 1656, 1657, 1658, 1659, 1660, 1661, 1662, 1665, 1666, 1667, 1669, 1670, 1672, 1673, 1674, 1675, 1676, 1677, 1680, 1681, 1682, 1683, 1685, 1689, 1690, 1692, 1693, 1695, 1698, 1700, 1701, 1703, 1704, 1705, 1706, 1707, 1709, 1710, 1711, 1712, 1713, 1714, 1715, 1716, 1717, 1718, 1719, 1720, 1722, 1723, 1724, 1726, 1727, 1734, 1735, 1736, 1739, 1740, 1746, 1747, 1748, 1749, 1750, 1751, 1752, 1753, 1754, 1755, 1757, 1758, 1759, 1762, 1763, 1764, 1765, 1767, 1768, 1772, 1774, 1775, 1776, 1778, 1779, 1780, 1781, 1782, 1783, 1784, 1787, 1790, 1791, 1793, 1794, 1795, 1797, 1799, 1800, 1802, 1808, 1809, 1811, 1812, 1814, 1816, 1817, 1818, 1820, 1821, 1822, 1823, 1827, 1828, 1829, 1831, 1834, 1836, 1838, 1839, 1843, 1844, 1849, 1851, 1853, 1854, 1867, 1869, 1871, 1873, 1875, 1878, 1879, 1881, 1885, 1886, 1887, 1889, 1891, 1894, 1897, 1898, 1899, 1900, 1901, 1903, 1904, 1906, 1913, 1914, 1916, 1917, 1922, 1925, 1926, 1927, 1929, 1931, 1932, 1934, 1936, 1937, 1938, 1939, 1942, 1944, 1946, 1947, 1948, 1950, 1951, 1952, 1953, 1955, 1956, 1957, 1959, 1960, 1961, 1962, 1963, 1964, 1965, 1968, 1969, 1970, 1971, 1972, 1973, 1974, 1975, 1976, 1977, 1978, 1979, 1980, 1981, 1982, 1983, 1984, 1985, 1986, 1987, 1988, 1989, 1990, 1991, 1992, 1993, 1994, 1995, 1996, 1997, 1998, 1999, 2000, 2001, 2002, 2003, 2004, 2005, 2006, 2007, 2008, 2009, 2010, 2011, 2012, 2013, 2014, 2015, 2016, 2017, 2018, 2019, 2020, 2021, and 2022. Such plasmid adapted sequences may further comprise a hairpin-forming motif between the PBS and the 3′ terminal U series.

Any of the PEgRNAs of Table 11 can be used in a Prime Editing system further comprising a nick guide RNA (ngRNA). Such ngRNA can comprise a spacer comprising nucleotides 5-20 of SEQ ID NO: 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 199, 200, 203, 529, 736, 737, 738, 739, or 740 and a gRNA core capable of binding to a Cas9 protein. The spacer of the ngRNA can comprise nucleotides 4-20, 3-20, 2-20, or 1-20 of SEQ ID NO: 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 199, 200, 203, 529, 736, 737, 738, 739, or 740. The spacer of the ngRNA can comprise SEQ ID NO: 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 199, 200, 203, 529, 736, 737, 738, 739, or 740. The gRNA core of the ngRNA can comprise SEQ ID NO. 5857-5859. Exemplary ngRNA provided in Table 11 can comprise SEQ ID NO: 153, 154, 155, 156, 157, 158, 159, 160, 161, 162, 163, 164, 165, 166, 167, 168, 169, 170, 171, 172, 173, 174, 175, 176, 177, 178, 179, 180, 181, 290, 291, 292, 293, 681, 1501, 1502, 1503, or 1504. Such ngRNA sequences may further comprise a 3′ motif at the 3′ end of the gRNA core, for example, a series of 3, 4 or more U nucleotides. Without being bound by theory, such 3′ motifs are believed to increase ngRNA stability.

Table 12 provides Prime Editing guide RNAs (PEgRNAs), which can be used in Prime Editing systems disclosed herein. Such Prime Editing systems can comprise a Cas9 protein capable of recognizing an NGG or NG PAM sequence (e.g., TGG or TG), and a reverse transcriptase. The Prime Editing systems (e.g., PE3 or PE3b systems) can further comprise a nick guide RNA (ngRNA). Such PEgRNAs and Prime Editing systems can be used, for example, to correct an H1069Q mutation in ATP7B.

The PEgRNAs of Table 12 comprise: (a) a spacer comprising nucleotides 5-20 of SEQ ID NO: 2023, (b) a gRNA core capable of binding to a Cas9 protein, and (c) an extension arm comprising: (i) an editing template comprising any one of SEQ ID NOs: 2035-2044, and (ii) a primer binding site (PBS) comprising any one of SEQ ID NOs: 2024-2034. The spacer of the PEgRNA can comprise, for example, nucleotides 4-20, 3-20, 2-20, or 1-20 of SEQ ID NO: 2023. The spacer of the PEgRNA can comprise SEQ ID NO: 2023. The RTT and the PBS can comprise respectively SEQ ID NO: 2035 and 2024, 2035 and 2025, 2035 and 2026, 2035 and 2027, 2035 and 2028, 2035 and 2029, 2035 and 2030, 2035 and 2031, 2035 and 2032, 2035 and 2033, 2035 and 2034, 2036 and 2024, 2036 and 2025, 2036 and 2026, 2036 and 2027, 2036 and 2028, 2036 and 2029, 2036 and 2030, 2036 and 2031, 2036 and 2032, 2036 and 2033, 2036 and 2034, 2037 and 2024, 2037 and 2025, 2037 and 2026, 2037 and 2027, 2037 and 2028, 2037 and 2029, 2037 and 2030, 2037 and 2031, 2037 and 2032, 2037 and 2033, 2037 and 2034, 2038 and 2024, 2038 and 2025, 2038 and 2026, 2038 and 2027, 2038 and 2028, 2038 and 2029, 2038 and 2030, 2038 and 2031, 2038 and 2032, 2038 and 2033, 2038 and 2034, 2039 and 2024, 2039 and 2025, 2039 and 2026, 2039 and 2027, 2039 and 2028, 2039 and 2029, 2039 and 2030, 2039 and 2031, 2039 and 2032, 2039 and 2033, 2039 and 2034, 2040 and 2024, 2040 and 2025, 2040 and 2026, 2040 and 2027, 2040 and 2028, 2040 and 2029, 2040 and 2030, 2040 and 2031, 2040 and 2032, 2040 and 2033, 2040 and 2034, 2041 and 2024, 2041 and 2025, 2041 and 2026, 2041 and 2027, 2041 and 2028, 2041 and 2029, 2041 and 2030, 2041 and 2031, 2041 and 2032, 2041 and 2033, 2041 and 2034, 2042 and 2024, 2042 and 2025, 2042 and 2026, 2042 and 2027, 2042 and 2028, 2042 and 2029, 2042 and 2030, 2042 and 2031, 2042 and 2032, 2042 and 2033, 2042 and 2034, 2043 and 2024, 2043 and 2025, 2043 and 2026, 2043 and 2027, 2043 and 2028, 2043 and 2029, 2043 and 2030, 2043 and 2031, 2043 and 2032, 2043 and 2033, 2043 and 2034, 2044 and 2024, 2044 and 2025, 2044 and 2026, 2044 and 2027, 2044 and 2028, 2044 and 2029, 2044 and 2030, 2044 and 2031, 2044 and 2032, 2044 and 2033, or 2044 and 2034. The gRNA core of the PEgRNA can comprise SEQ ID NO. 5857-5859. Exemplary PEgRNAs provided in Table 12 can comprise SEQ ID NO. 2097, 2098, 2099, 2100, 2101, 2102, 2103, 2104, 2105, 2106, 2107, 2108, 2109, 2110, 2111, 2112, 2113, 2114, 2115, 2116, 2117, 2118, 2119, 2120, 2121, 2122, 2123, 2124, 2125, 2126, 2127, 2128, 2129, 2131, 2132, 2133, 2134, 2135, 2136, 2137, 2138, 2140, 2142, 2144, 2145, 2146, 2147, 2148, 2149, 2150, 2151, 2154, 2155, 2156, 2158, 2160, 2161, 2165, 2166, 2167, 2168, 2169, 2170, 2171, 2174, 2178, 2179, 2183, 2187, 2194, 2195, 2197, 2199, 2200, 2202, 2207, 2208, 2220, 2226, or 2232. Such PEgRNA sequences may further comprise a 3Y motif at the 3′ end of the extension arm, for example, a hairpin-forming motif or a series of 3, 4 or more U nucleotides. Without being bound by theory, such 3′ motifs are believed to increase PEgRNA stability. Such PEgRNA sequences may alternatively or additionally be adapted for transcription from a DNA template, for example, by including a 5′ terminal G if the spacer of the PEgRNA begins with another nucleotide, by including 6 or 7 U nucleotides at the 3′ end of the extension arm, or both. Exemplary transcription-adapted sequences include SEQ ID NOs: 2130, 2139, 2141, 2143, 2152, 2153, 2157, 2159, 2162, 2163, 2164, 2172, 2173, 2175, 2176, 2177, 2180, 2181, 2182, 2184, 2185, 2186, 2188, 2189, 2190, 2191, 2192, 2193, 2196, 2198, 2201, 2203, 2204, 2205, 2206, 2209, 2210, 2211, 2212, 2213, 2214, 2215, 2216, 2217, 2218, 2219, 2221, 2222, 2223, 2224, 2225, 2227, 2228, 2229, 2230, 2231, 2233, 2234, 2235, 2236, 2237, 2238, 2239, 2240, 2241, 2242, 2243, 2244, 2245, 2246, 2247, 2248, 2249, 2250, 2251, 2252, 2253, 2254, 2255, and 2256. Such plasmid adapted sequences may further comprise a hairpin-forming motif between the PBS and the 3′ terminal U series.

Any of the PEgRNAs of Table 12 can be used in a Prime Editing system further comprising a nick guide RNA (ngRNA). Such ngRNA can comprise a spacer comprising nucleotides 5-20 of SEQ ID NO: 41, 60, 61, 62, 63, 64, 65, 66, 69, 71, 2045, 2046, 2047, 2048, 2049, 2050, 2051, 2052, 2053, 2054, 2055, 2056, 2057, 2058, 2059, 2060, 2061, 2062, 2063, 2064, 2065, 2066, 2067, 2068, 2069, 2070, 2071, 2072, 2073, 2074, 2075, 2076, 2077, 2078, 2079, 2080, 2081, 2082, 2083, 2084, 2085, 2086, 2087, 2088, 2089, 2090, 2091, 2092, 2093, 2094, 2095, or 2096 and a gRNA core capable of binding to a Cas9 protein. The spacer of the ngRNA can comprise nucleotides 4-20, 3-20, 2-20, or 1-20 of SEQ ID NO: 41, 60, 61, 62, 63, 64, 65, 66, 69, 71, 2045, 2046, 2047, 2048, 2049, 2050, 2051, 2052, 2053, 2054, 2055, 2056, 2057, 2058, 2059, 2060, 2061, 2062, 2063, 2064, 2065, 2066, 2067, 2068, 2069, 2070, 2071, 2072, 2073, 2074, 2075, 2076, 2077, 2078, 2079, 2080, 2081, 2082, 2083, 2084, 2085, 2086, 2087, 2088, 2089, 2090, 2091, 2092, 2093, 2094, 2095, or 2096. The spacer of the ngRNA can comprise SEQ ID NO: 41, 60, 61, 62, 63, 64, 65, 66, 69, 71, 2045, 2046, 2047, 2048, 2049, 2050, 2051, 2052, 2053, 2054, 2055, 2056, 2057, 2058, 2059, 2060, 2061, 2062, 2063, 2064, 2065, 2066, 2067, 2068, 2069, 2070, 2071, 2072, 2073, 2074, 2075, 2076, 2077, 2078, 2079, 2080, 2081, 2082, 2083, 2084, 2085, 2086, 2087, 2088, 2089, 2090, 2091, 2092, 2093, 2094, 2095, or 2096. The gRNA core of the ngRNA can comprise SEQ ID NO. 5857-5859. Exemplary ngRNA provided in Table 12 can comprise any one of SEQ ID NOs: 2257-2289. Such ngRNA sequences may further comprise a 3′ motif at the 3′ end of the gRNA core, for example, a series of 3, 4 or more U nucleotides. Exemplary ngRNA sequences with such 3′ adaptations include SEQ ID NOs: 2290-2292. Without being bound by theory, such 3′ motifs are believed to increase ngRNA stability.

Table 15 provides Prime Editing guide RNAs (PEgRNAs), which can be used in Prime Editing systems disclosed herein. Such Prime Editing systems can comprise a Cas9 protein capable of recognizing an NGG, NG, or NNGG PAM sequence (e.g., AGG, AG, or AGGG), and a reverse transcriptase. The Prime Editing systems (e.g., PE3 or PE3b systems) can further comprise a nick guide RNA (ngRNA). Such PEgRNAs and Prime Editing systems can be used, for example, to correct an H1069Q mutation in ATP7B.

The PEgRNAs of Table 15 comprise: (a) a spacer comprising nucleotides 5-20 of SEQ ID NO: 2293, (b) a gRNA core capable of binding to a Cas9 protein, and (c) an extension arm comprising: (i) an editing template comprising any one of SEQ ID NOs: 2305-2422, and (ii) a primer binding site (PBS) comprising any one of SEQ ID NOs: 2294-2304. The spacer of the PEgRNA can comprise, for example, nucleotides 4-20, 3-20, 2-20, or 1-20 of SEQ ID NO: 2293. The spacer of the PEgRNA can comprise SEQ ID NO: 2293. The RTT and the PBS can comprise respectively SEQ ID NOs: 2305 and 2294, 2305 and 2295, 2305 and 2296, 2305 and 2297, 2305 and 2298, 2305 and 2299, 2305 and 2300, 2305 and 2301, 2305 and 2302, 2305 and 2303, 2305 and 2304, 2306 and 2294, 2306 and 2295, 2306 and 2296, 2306 and 2297, 2306 and 2298, 2306 and 2299, 2306 and 2300, 2306 and 2301, 2306 and 2302, 2306 and 2303, 2306 and 2304, 2307 and 2294, 2307 and 2295, 2307 and 2296, 2307 and 2297, 2307 and 2298, 2307 and 2299, 2307 and 2300, 2307 and 2301, 2307 and 2302, 2307 and 2303, 2307 and 2304, 2308 and 2294, 2308 and 2295, 2308 and 2296, 2308 and 2297, 2308 and 2298, 2308 and 2299, 2308 and 2300, 2308 and 2301, 2308 and 2302, 2308 and 2303, 2308 and 2304, 2309 and 2294, 2309 and 2295, 2309 and 2296, 2309 and 2297, 2309 and 2298, 2309 and 2299, 2309 and 2300, 2309 and 2301, 2309 and 2302, 2309 and 2303, 2309 and 2304, 2310 and 2294, 2310 and 2295, 2310 and 2296, 2310 and 2297, 2310 and 2298, 2310 and 2299, 2310 and 2300, 2310 and 2301, 2310 and 2302, 2310 and 2303, 2310 and 2304, 2311 and 2294, 2311 and 2295, 2311 and 2296, 2311 and 2297, 2311 and 2298, 2311 and 2299, 2311 and 2300, 2311 and 2301, 2311 and 2302, 2311 and 2303, 2311 and 2304, 2312 and 2294, 2312 and 2295, 2312 and 2296, 2312 and 2297, 2312 and 2298, 2312 and 2299, 2312 and 2300, 2312 and 2301, 2312 and 2302, 2312 and 2303, 2312 and 2304, 2313 and 2294, 2313 and 2295, 2313 and 2296, 2313 and 2297, 2313 and 2298, 2313 and 2299, 2313 and 2305, 2313 and 2301, 2313 and 2302, 2313 and 2303, 2313 and 2304, 2314 and 2294, 2314 and 2295, 2314 and 2296, 2314 and 2297, 2314 and 2298, 2314 and 2299, 2314 and 2300, 2314 and 2301, 2314 and 2302, 2314 and 2303, 2314 and 2304, 2315 and 2294, 2315 and 2295, 2315 and 2296, 2315 and 2297, 2315 and 2298, 2315 and 2299, 2315 and 2300, 2315 and 2301, 2315 and 2302, 2315 and 2303, 2315 and 2304, 2316 and 2294, 2316 and 2295, 2316 and 2296, 2316 and 2297, 2316 and 2298, 2316 and 2299, 2316 and 2300, 2316 and 2301, 2316 and 2302, 2316 and 2303, 2316 and 2304, 2317 and 2294, 2317 and 2295, 2317 and 2296, 2317 and 2297, 2317 and 2298, 2317 and 2299, 2317 and 2300, 2317 and 2301, 2317 and 2302, 2317 and 2303, 2317 and 2304, 2318 and 2294, 2318 and 2295, 2318 and 2296, 2318 and 2297, 2318 and 2298, 2318 and 2299, 2318 and 2300, 2318 and 2301, 2318 and 2302, 2318 and 2303, 2318 and 2304, 2319 and 2294, 2319 and 2295, 2319 and 2296, 2319 and 2297, 2319 and 2298, 2319 and 2299, 2319 and 2300, 2319 and 2301, 2319 and 2302, 2319 and 2303, 2319 and 2304, 2320 and 2294, 2320 and 2295, 2320 and 2296, 2320 and 2297, 2320 and 2298, 2320 and 2299, 2320 and 2300, 2320 and 2301, 2320 and 2302, 2320 and 2303, 2320 and 2304, 2321 and 2294, 2321 and 2295, 2321 and 2296, 2321 and 2297, 2321 and 2298, 2321 and 2299, 2321 and 2300, 2321 and 2301, 2321 and 2302, 2321 and 2303, 2321 and 2304, 2322 and 2294, 2322 and 2295, 2322 and 2296, 2322 and 2297, 2322 and 2298, 2322 and 2299, 2322 and 2300, 2322 and 2301, 2322 and 2302, 2322 and 2303, 2322 and 2304, 2323 and 2294, 2323 and 2295, 2323 and 2296, 2323 and 2297, 2323 and 2298, 2323 and 2299, 2323 and 23004, 2323 and 2301, 2323 and 2302, 2323 and 2303, 2323 and 2304, 2324 and 2294, 2324 and 2295, 2324 and 2296, 2324 and 2297, 2324 and 2298, 2324 and 2299, 2324 and 2300, 2324 and 2301, 2324 and 2302, 2324 and 2303, 2324 and 2304, 2325 and 2294, 2325 and 2295, 2325 and 2296, 2325 and 2297, 2325 and 2298, 2325 and 2299, 2325 and 2300, 2325 and 2301, 2325 and 2302, 2325 and 2303, 2325 and 2304, 2326 and 2294, 2326 and 2295, 2326 and 2296, 2326 and 2297, 2326 and 2298, 2326 and 2299, 2326 and 2300, 2326 and 2301, 2326 and 2302, 2326 and 2303, 2326 and 2304, 2327 and 2294, 2327 and 2295, 2327 and 2296, 2327 and 2297, 2327 and 2298, 2327 and 2299, 2327 and 2300, 2327 and 2301, 2327 and 2302, 2327 and 2303, 2327 and 2304, 2328 and 2294, 2328 and 2295, 2328 and 2296, 2328 and 2297, 2328 and 2298, 2328 and 2299, 2328 and 2300, 2328 and 2301, 2328 and 2302, 2328 and 2303, 2328 and 2304, 2329 and 2294, 2329 and 2295, 2329 and 2296, 2329 and 2297, 2329 and 2298, 2329 and 2299, 2329 and 2300, 2329 and 2301, 2329 and 2302, 2329 and 2303, 2329 and 2304, 2330 and 2294, 2330 and 2295, 2330 and 2296, 2330 and 2297, 2330 and 2298, 2330 and 2299, 2330 and 2300, 2330 and 2301, 2330 and 2302, 2330 and 2303, 2330 and 2304, 2331 and 2294, 2331 and 2295, 2331 and 2296, 2331 and 2297, 2331 and 2298, 2331 and 2299, 2331 and 2300, 2331 and 2301, 2331 and 2302, 2331 and 2303, 2331 and 2304, 2332 and 2294, 2332 and 2295, 2332 and 2296, 2332 and 2297, 2332 and 2298, 2332 and 2299, 2332 and 2300, 2332 and 2301, 2332 and 2302, 2332 and 2303, 2332 and 2304, 2333 and 2294, 2333 and 2295, 2333 and 2296, 2333 and 2297, 2333 and 2298, 2333 and 2299, 2333 and 2300, 2333 and 2301, 2333 and 2302, 2333 and 2303, 2333 and 2304, 2334 and 2294, 2334 and 2295, 2334 and 2296, 2334 and 2297, 2334 and 2298, 2334 and 2299, 2334 and 2300, 2334 and 2301, 2334 and 2302, 2334 and 2303, 2334 and 2304, 2335 and 2294, 2335 and 2295, 2335 and 2296, 2335 and 2297, 2335 and 2298, 2335 and 2299, 2335 and 2300, 2335 and 2301, 2335 and 2302, 2335 and 2303, 2335 and 2304, 2336 and 2294, 2336 and 2295, 2336 and 2296, 2336 and 2297, 2336 and 2298, 2336 and 2299, 2336 and 2300, 2336 and 2301, 2336 and 2302, 2336 and 2303, 2336 and 2304, 2337 and 2294, 2337 and 2295, 2337 and 2296, 2337 and 2297, 2337 and 2298, 2337 and 2299, 2337 and 2300, 2337 and 2301, 2337 and 2302, 2337 and 2303, 2337 and 2304, 2338 and 2294, 2338 and 2295, 2338 and 2296, 2338 and 2297, 2338 and 2298, 2338 and 2299, 2338 and 2300, 2338 and 2301, 2338 and 2302, 2338 and 2303, 2338 and 2304, 2339 and 2294, 2339 and 2295, 2339 and 2296, 2339 and 2297, 2339 and 2298, 2339 and 2299, 2339 and 2300, 2339 and 2301, 2339 and 2302, 2339 and 2303, 2339 and 2304, 2340 and 2294, 2340 and 2295, 2340 and 2296, 2340 and 2297, 2340 and 2298, 2340 and 2299, 2340 and 2300, 2340 and 2301, 2340 and 2302, 2340 and 2303, 2340 and 2304, 2341 and 2294, 2341 and 2295, 2341 and 2296, 2341 and 2297, 2341 and 2298, 2341 and 2299, 2341 and 2300, 2341 and 2301, 2341 and 2302, 2341 and 2303, 2341 and 2304, 2342 and 2294, 2342 and 2295, 2342 and 2296, 2342 and 2297, 2342 and 2298, 2342 and 2299, 2342 and 2300, 2342 and 2301, 2342 and 2302, 2342 and 2303, 2342 and 2304, 2343 and 2294, 2343 and 2295, 2343 and 2296, 2343 and 2297, 2343 and 2298, 2343 and 2299, 2343 and 2300, 2343 and 2301, 2343 and 2302, 2343 and 2303, 2343 and 2304, 2344 and 2294, 2344 and 2295, 2344 and 2296, 2344 and 2297, 2344 and 2298, 2344 and 2299, 2344 and 2300, 2344 and 2301, 2344 and 2302, 2344 and 2303, 2344 and 2304, 2345 and 2294, 2345 and 2295, 2345 and 2296, 2345 and 2297, 2345 and 2298, 2345 and 2299, 2345 and 2300, 2345 and 2301, 2345 and 2302, 2345 and 2303, 2345 and 2304, 2346 and 2294, 2346 and 2295, 2346 and 2296, 2346 and 2297, 2346 and 2298, 2346 and 2299, 2346 and 2300, 2346 and 2301, 2346 and 2302, 2346 and 2303, 2346 and 2304, 2347 and 2294, 2347 and 2295, 2347 and 2296, 2347 and 2297, 2347 and 2298, 2347 and 2299, 2347 and 2300, 2347 and 2301, 2347 and 2302, 2347 and 2303, 2347 and 2304, 2348 and 2294, 2348 and 2295, 2348 and 2296, 2348 and 2297, 2348 and 2298, 2348 and 2299, 2348 and 2300, 2348 and 2301, 2348 and 2302, 2348 and 2303, 2348 and 2304, 2349 and 2294, 2349 and 2295, 2349 and 2296, 2349 and 2297, 2349 and 2298, 2349 and 2299, 2349 and 230, 2349 and 2301, 2349 and 2302, 2349 and 2303, 2349 and 2304, 2350 and 2294, 2350 and 2295, 2350 and 2296, 2350 and 2297, 2350 and 2298, 2350 and 2299, 2350 and 2300, 2350 and 2301, 2350 and 2302, 2350 and 2303, 2350 and 2304, 2351 and 2294, 2351 and 2295, 2351 and 2296, 2351 and 2297, 2351 and 2298, 2351 and 2299, 2351 and 2300, 2351 and 2301, 2351 and 2302, 2351 and 2303, 2351 and 2304, 2352 and 2294, 2352 and 2295, 2352 and 2296, 2352 and 2297, 2352 and 2298, 2352 and 2299, 2352 and 2300, 2352 and 2301, 2352 and 2302, 2352 and 2303, 2352 and 2304, 2353 and 2294, 2353 and 2295, 2353 and 2296, 2353 and 2297, 2353 and 2298, 2353 and 2299, 2353 and 2300, 2353 and 2301, 2353 and 2302, 2353 and 2303, 2353 and 2304, 2354 and 2294, 2354 and 2295, 2354 and 2296, 2354 and 2297, 2354 and 2298, 2354 and 2299, 2354 and 2300, 2354 and 2301, 2354 and 2302, 2354 and 2303, 2354 and 2304, 2355 and 2294, 2355 and 2295, 2355 and 2296, 2355 and 2297, 2355 and 2298, 2355 and 2299, 2355 and 2300, 2355 and 2301, 2355 and 2302, 2355 and 2303, 2355 and 2304, 2356 and 2294, 2356 and 2295, 2356 and 2296, 2356 and 2297, 2356 and 2298, 2356 and 2299, 2356 and 2300, 2356 and 2301, 2356 and 2302, 2356 and 2303, 2356 and 2304, 2357 and 2294, 2357 and 2295, 2357 and 2296, 2357 and 2297, 2357 and 2298, 2357 and 2299, 2357 and 2300, 2357 and 2301, 2357 and 2302, 2357 and 2303, 2357 and 2304, 2358 and 2294, 2358 and 2295, 2358 and 2296, 2358 and 2297, 2358 and 2298, 2358 and 2299, 2358 and 2300, 2358 and 2301, 2358 and 2302, 2358 and 2303, 2358 and 2304, 2359 and 2294, 2359 and 2295, 2359 and 2296, 2359 and 2297, 2359 and 2298, 2359 and 2299, 2359 and 2300, 2359 and 2301, 2359 and 2302, 2359 and 2303, 2359 and 2304, 2360 and 2294, 2360 and 2295, 2360 and 2296, 2360 and 2297, 2360 and 2298, 2360 and 2299, 2360 and 2300, 2360 and 2301, 2360 and 2302, 2360 and 2303, 2360 and 2304, 2361 and 2294, 2361 and 2295, 2361 and 2296, 2361 and 2297, 2361 and 2298, 2361 and 2299, 2361 and 2300, 2361 and 2301, 2361 and 2302, 2361 and 2303, 2361 and 2304, 2362 and 2294, 2362 and 2295, 2362 and 2296, 2362 and 2297, 2362 and 2298, 2362 and 2299, 2362 and 2300, 2362 and 2301, 2362 and 2302, 2362 and 2303, 2362 and 2304, 2363 and 2294, 2363 and 2295, 2363 and 2296, 2363 and 2297, 2363 and 2298, 2363 and 2299, 2363 and 2300, 2363 and 2301, 2363 and 2302, 2363 and 2303, 2363 and 2304, 2364 and 2294, 2364 and 2295, 2364 and 2296, 2364 and 2297, 2364 and 2298, 2364 and 2299, 2364 and 2300, 2364 and 2301, 2364 and 2302, 2364 and 2303, 2364 and 2304, 2365 and 2294, 2365 and 2295, 2365 and 2296, 2365 and 2297, 2365 and 2298, 2365 and 2299, 2365 and 2300, 2365 and 2301, 2365 and 2302, 2365 and 2303, 2365 and 2304, 2366 and 2294, 2366 and 2295, 2366 and 2296, 2366 and 2297, 2366 and 2298, 2366 and 2299, 2366 and 2300, 2366 and 2301, 2366 and 2302, 2366 and 2303, 2366 and 2304, 2367 and 2294, 2367 and 2295, 2367 and 2296, 2367 and 2297, 2367 and 2298, 2367 and 2299, 2367 and 2300, 2367 and 2301, 2367 and 2302, 2367 and 2303, 2367 and 2304, 2368 and 2294, 2368 and 2295, 2368 and 2296, 2368 and 2297, 2368 and 2298, 2368 and 2299, 2368 and 2300, 2368 and 2301, 2368 and 2302, 2368 and 2303, 2368 and 2304, 2369 and 2294, 2369 and 2295, 2369 and 2296, 2369 and 2297, 2369 and 2298, 2369 and 2299, 2369 and 2300, 2369 and 2301, 2369 and 2302, 2369 and 2303, 2369 and 2304, 2370 and 2294, 2370 and 2295, 2370 and 2296, 2370 and 2297, 2370 and 2298, 2370 and 2299, 2370 and 2300, 2370 and 2301, 2370 and 2302, 2370 and 2303, 2370 and 2304, 2371 and 2294, 2371 and 2295, 2371 and 2296, 2371 and 2297, 2371 and 2298, 2371 and 2299, 2371 and 2300, 2371 and 2301, 2371 and 2302, 2371 and 2303, 2371 and 2304, 2372 and 2294, 2372 and 2295, 2372 and 2296, 2372 and 2297, 2372 and 2298, 2372 and 2299, 2372 and 2300, 2372 and 2301, 2372 and 2302, 2372 and 2303, 2372 and 2304, 2373 and 2294, 2373 and 2295, 2373 and 2296, 2373 and 2297, 2373 and 2298, 2373 and 2299, 2373 and 2300, 2373 and 2301, 2373 and 2302, 2373 and 2303, 2373 and 2304, 2374 and 2294, 2374 and 2295, 2374 and 2296, 2374 and 2297, 2374 and 2298, 2374 and 2299, 2374 and 2300, 2374 and 2301, 2374 and 2302, 2374 and 2303, 2374 and 2304, 2375 and 2294, 2375 and 2295, 2375 and 2296, 2375 and 2297, 2375 and 2298, 2375 and 2299, 2375 and 2300, 2375 and 2301, 2375 and 2302, 2375 and 2303, 2375 and 2304, 2376 and 2294, 2376 and 2295, 2376 and 2296, 2376 and 2297, 2376 and 2298, 2376 and 2299, 2376 and 2300, 2376 and 2301, 2376 and 2302, 2376 and 2303, 2376 and 2304, 2377 and 2294, 2377 and 2295, 2377 and 2296, 2377 and 2297, 2377 and 2298, 2377 and 2299, 2377 and 2300, 2377 and 2301, 2377 and 2302, 2377 and 2303, 2377 and 2304, 2378 and 2294, 2378 and 2295, 2378 and 2296, 2378 and 2297, 2378 and 2298, 2378 and 2299, 2378 and 2300, 2378 and 2301, 2378 and 2302, 2378 and 2303, 2378 and 2304, 2379 and 2294, 2379 and 2295, 2379 and 2296, 2379 and 2297, 2379 and 2298, 2379 and 2299, 2379 and 2300, 2379 and 2301, 2379 and 2302, 2379 and 2303, 2379 and 2304, 2380 and 2294, 2380 and 2295, 2380 and 2296, 2380 and 2297, 2380 and 2298, 2380 and 2299, 2380 and 2300, 2380 and 2301, 2380 and 2302, 2380 and 2303, 2380 and 2304, 2381 and 2294, 2381 and 2295, 2381 and 2296, 2381 and 2297, 2381 and 2298, 2381 and 2299, 2381 and 2300, 2381 and 2301, 2381 and 2302, 2381 and 2303, 2381 and 2304, 2382 and 2294, 2382 and 2295, 2382 and 2296, 2382 and 2297, 2382 and 2298, 2382 and 2299, 2382 and 2300, 2382 and 2301, 2382 and 2302, 2382 and 2303, 2382 and 2304, 2383 and 2294, 2383 and 2295, 2383 and 2296, 2383 and 2297, 2383 and 2298, 2383 and 2299, 2383 and 2300, 2383 and 2301, 2383 and 2302, 2383 and 2303, 2383 and 2304, 2384 and 2294, 2384 and 2295, 2384 and 2296, 2384 and 2297, 2384 and 2298, 2384 and 2299, 2384 and 2300, 2384 and 2301, 2384 and 2302, 2384 and 2303, 2384 and 2304, 2385 and 2294, 2385 and 2295, 2385 and 2296, 2385 and 2297, 2385 and 2298, 2385 and 2299, 2385 and 2300, 2385 and 2301, 2385 and 2302, 2385 and 2303, 2385 and 2304, 2386 and 2294, 2386 and 2295, 2386 and 2296, 2386 and 2297, 2386 and 2298, 2386 and 2299, 2386 and 2300, 2386 and 2301, 2386 and 2302, 2386 and 2303, 2386 and 2304, 2387 and 2294, 2387 and 2295, 2387 and 2296, 2387 and 2297, 2387 and 2298, 2387 and 2299, 2387 and 2300, 2387 and 2301, 2387 and 2302, 2387 and 2303, 2387 and 2304, 2388 and 2294, 2388 and 2295, 2388 and 2296, 2388 and 2297, 2388 and 2298, 2388 and 2299, 2388 and 2300, 2388 and 2301, 2388 and 2302, 2388 and 2303, 2388 and 2304, 2389 and 2294, 2389 and 2295, 2389 and 2296, 2389 and 2297, 2389 and 2298, 2389 and 2299, 2389 and 2300, 2389 and 2301, 2389 and 2302, 2389 and 2303, 2389 and 2304, 2390 and 2294, 2390 and 2295, 2390 and 2296, 2390 and 2297, 2390 and 2298, 2390 and 2299, 2390 and 2300, 2390 and 2301, 2390 and 2302, 2390 and 2303, 2390 and 2304, 2391 and 2294, 2391 and 2295, 2391 and 2296, 2391 and 2297, 2391 and 2298, 2391 and 2299, 2391 and 2300, 2391 and 2301, 2391 and 2302, 2391 and 2303, 2391 and 2304, 2392 and 2294, 2392 and 2295, 2392 and 2296, 2392 and 2297, 2392 and 2298, 2392 and 2299, 2392 and 2300, 2392 and 2301, 2392 and 2302, 2392 and 2303, 2392 and 2304, 2393 and 2294, 2393 and 2295, 2393 and 2296, 2393 and 2297, 2393 and 2298, 2393 and 2299, 2393 and 2300, 2393 and 2301, 2393 and 2302, 2393 and 2303, 2393 and 2304, 2394 and 2294, 2394 and 2295, 2394 and 2296, 2394 and 2297, 2394 and 2298, 2394 and 2299, 2394 and 2300, 2394 and 2301, 2394 and 2302, 2394 and 2303, 2394 and 2304, 2395 and 2294, 2395 and 2295, 2395 and 2296, 2395 and 2297, 2395 and 2298, 2395 and 2299, 2395 and 2300, 2395 and 2301, 2395 and 2302, 2395 and 2303, 2395 and 2304, 2396 and 2294, 2396 and 2295, 2396 and 2296, 2396 and 2297, 2396 and 2298, 2396 and 2299, 2396 and 2300, 2396 and 2301, 2396 and 2302, 2396 and 2303, 2396 and 2304, 2397 and 2294, 2397 and 2295, 2397 and 2296, 2397 and 2297, 2397 and 2298, 2397 and 2299, 2397 and 2300, 2397 and 2301, 2397 and 2302, 2397 and 2303, 2397 and 2304, 2398 and 2294, 2398 and 2295, 2398 and 2296, 2398 and 2297, 2398 and 2298, 2398 and 2299, 2398 and 2300, 2398 and 2301, 2398 and 2302, 2398 and 2303, 2398 and 2304, 2399 and 2294, 2399 and 2295, 2399 and 2296, 2399 and 2297, 2399 and 2298, 2399 and 2299, 2399 and 2300, 2399 and 2301, 2399 and 2302, 2399 and 2303, 2399 and 2304, 2400 and 2294, 2400 and 2295, 2400 and 2296, 2400 and 2297, 2400 and 2298, 2400 and 2299, 2400 and 2300, 2400 and 2301, 2400 and 2302, 2400 and 2303, 2400 and 2304, 2401 and 2294, 2401 and 2295, 2401 and 2296, 2401 and 2297, 2401 and 2298, 2401 and 2299, 2401 and 2300, 2401 and 2301, 2401 and 2302, 2401 and 2303, 2401 and 2304, 2402 and 2294, 2402 and 2295, 2402 and 2296, 2402 and 2297, 2402 and 2298, 2402 and 2299, 2402 and 2300, 2402 and 2301, 2402 and 2302, 2402 and 2303, 2402 and 2304, 2403 and 2294, 2403 and 2295, 2403 and 2296, 2403 and 2297, 2403 and 2298, 2403 and 2299, 2403 and 2300, 2403 and 2301, 2403 and 2302, 2403 and 2303, 2403 and 2304, 2404 and 2294, 2404 and 2295, 2404 and 2296, 2404 and 2297, 2404 and 2298, 2404 and 2299, 2404 and 2300, 2404 and 2301, 2404 and 2302, 2404 and 2303, 2404 and 2304, 2405 and 2294, 2405 and 2295, 2405 and 2296, 2405 and 2297, 2405 and 2298, 2405 and 2299, 2405 and 2300, 2405 and 2301, 2405 and 2302, 2405 and 2303, 2405 and 2304, 2406 and 2294, 2406 and 2295, 2406 and 2296, 2406 and 2297, 2406 and 2298, 2406 and 2299, 2406 and 2300, 2406 and 2301, 2406 and 2302, 2406 and 2303, 2406 and 2304, 2407 and 2294, 2407 and 2295, 2407 and 2296, 2407 and 2297, 2407 and 2298, 2407 and 2299, 2407 and 2300, 2407 and 2301, 2407 and 2302, 2407 and 2303, 2407 and 2304, 2408 and 2294, 2408 and 2295, 2408 and 2296, 2408 and 2297, 2408 and 2298, 2408 and 2299, 2408 and 2300, 2408 and 2301, 2408 and 2302, 2408 and 2303, 2408 and 2304, 2409 and 2294, 2409 and 2295, 2409 and 2296, 2409 and 2297, 2409 and 2298, 2409 and 2299, 2409 and 2300, 2409 and 2301, 2409 and 2302, 2409 and 2303, 2409 and 2304, 2410 and 2294, 2410 and 2295, 2410 and 2296, 2410 and 2297, 2410 and 2298, 2410 and 2299, 2410 and 2300, 2410 and 2301, 2410 and 2302, 2410 and 2303, 2410 and 2304, 2411 and 2294, 2411 and 2295, 2411 and 2296, 2411 and 2297, 2411 and 2298, 2411 and 2299, 2411 and 2300, 2411 and 2301, 2411 and 2302, 2411 and 2303, 2411 and 2304, 2412 and 2294, 2412 and 2295, 2412 and 2296, 2412 and 2297, 2412 and 2298, 2412 and 2299, 2412 and 2300, 2412 and 2301, 2412 and 2302, 2412 and 2303, 2412 and 2304, 2413 and 2294, 2413 and 2295, 2413 and 2296, 2413 and 2297, 2413 and 2298, 2413 and 2299, 2413 and 2300, 2413 and 2301, 2413 and 2302, 2413 and 2303, 2413 and 2304, 2414 and 2294, 2414 and 2295, 2414 and 2296, 2414 and 2297, 2414 and 2298, 2414 and 2299, 2414 and 2300, 2414 and 2301, 2414 and 2302, 2414 and 2303, 2414 and 2304, 2415 and 2294, 2415 and 2295, 2415 and 2296, 2415 and 2297, 2415 and 2298, 2415 and 2299, 2415 and 2300, 2415 and 2301, 2415 and 2302, 2415 and 2303, 2415 and 2304, 2416 and 2294, 2416 and 2295, 2416 and 2296, 2416 and 2297, 2416 and 2298, 2416 and 2299, 2416 and 2300, 2416 and 2301, 2416 and 2302, 2416 and 2303, 2416 and 2304, 2417 and 2294, 2417 and 2295, 2417 and 2296, 2417 and 2297, 2417 and 2298, 2417 and 2299, 2417 and 2300, 2417 and 2301, 2417 and 2302, 2417 and 2303, 2417 and 2304, 2418 and 2294, 2418 and 2295, 2418 and 2296, 2418 and 2297, 2418 and 2298, 2418 and 2299, 2418 and 2300, 2418 and 2301, 2418 and 2302, 2418 and 2303, 2418 and 2304, 2419 and 2294, 2419 and 2295, 2419 and 2296, 2419 and 2297, 2419 and 2298, 2419 and 2299, 2419 and 2300, 2419 and 2301, 2419 and 2302, 2419 and 2303, 2419 and 2304, 2420 and 2294, 2420 and 2295, 2420 and 2296, 2420 and 2297, 2420 and 2298, 2420 and 2299, 2420 and 2300, 2420 and 2301, 2420 and 2302, 2420 and 2303, 2420 and 2304, 2421 and 2294, 2421 and 2295, 2421 and 2296, 2421 and 2297, 2421 and 2298, 2421 and 2299, 2421 and 2300, 2421 and 2301, 2421 and 2302, 2421 and 2303, 2421 and 2304, 2422 and 2294, 2422 and 2295, 2422 and 2296, 2422 and 2297, 2422 and 2298, 2422 and 2299, 2422 and 2300, 2422 and 2301, 2422 and 2302, 2422 and 2303, or 2422 and 2304, The gRNA core of the PEgRNA can comprise SEQ ID NO. 5857-5859. Exemplary PEgRNAs provided in Table 15 can comprise SEQ ID NO. 2445, 2446, 2447, 2448, 2449, 2450, 2451, 2452, 2453, 2454, 2455, 2456, 2457, 2458, 2459, 2460, 2461, 2462, 2463, 2464, 2465, 2466, 2467, 2468, 2469, 2470, 2471, 2472, 2473, 2474, 2475, 2476, 2477, 2478, 2479, 2480, 2481, 2482, 2483, 2484, 2485, 2486, 2487, 2488, 2489, 2490, 2491, 2492, 2493, 2494, 2495, 2496, 2497, 2498, 2499, 2500, 2501, 2502, 2503, 2504, 2505, 2506, 2507, 2508, 2509, 2510, 2511, 2512, 2513, 2514, 2515, 2516, 2517, 2518, 2519, 2520, 2521, 2522, 2523, 2524, 2525, 2526, 2527, 2528, 2529, 2530, 2531, 2532, 2533, 2534, 2535, 2538, 2539, 2540, 2541, 2542, 2543, 2544, 2545, 2546, 2547, 2548, 2549, 2550, 2551, 2553, 2554, 2555, 2556, 2557, 2558, 2559, 2560, 2561, 2562, 2563, 2564, 2565, 2566, 2567, 2568, 2569, 2570, 2571, 2572, 2573, 2574, 2575, 2576, 2577, 2578, 2580, 2582, 2584, 2585, 2586, 2587, 2588, 2589, 2590, 2591, 2592, 2593, 2594, 2595, 2596, 2597, 2598, 2600, 2601, 2602, 2603, 2605, 2606, 2607, 2608, 2609, 2610, 2611, 2612, 2613, 2614, 2615, 2616, 2617, 2618, 2619, 2620, 2621, 2623, 2624, 2628, 2629, 2630, 2631, 2632, 2633, 2634, 2635, 2636, 2637, 2638, 2643, 2645, 2646, 2647, 2648, 2649, 2650, 2651, 2652, 2653, 2654, 2655, 2656, 2657, 2658, 2659, 2660, 2663, 2664, 2665, 2667, 2668, 2669, 2670, 2671, 2672, 2674, 2675, 2676, 2677, 2678, 2680, 2681, 2683, 2685, 2687, 2688, 2689, 2690, 2692, 2694, 2695, 2696, 2697, 2699, 2701, 2702, 2704, 2706, 2708, 2711, 2713, 2715, 2716, 2717, 2720, 2721, 2722, 2723, 2725, 2726, 2727, 2728, 2729, 2730, 2733, 2734, 2735, 2744, 2747, 2748, 2749, 2752, 2753, 2757, 2758, 2759, 2760, 2761, 2762, 2764, 2765, 2768, 2769, 2770, 2772, 2773, 2774, 2777, 2786, 2788, 2789, 2790, 2791, 2792, 2793, 2794, 2795, 2796, 2797, 2798, 2799, 2800, 2801, 2802, 2807, 2810, 2811, 2812, 2814, 2816, 2824, 2825, 2826, 2828, 2829, 2830, 2832, 2833, 2834, 2841, 2842, 2843, 2844, 2846, 2847, 2854, 2855, 2856, 2857, 2862, 2864, 2866, 2867, 2868, 2869, 2870, 2871, 2885, 2886, 2887, 2888, 2889, 2890, 2891, 2893, 2894, 2896, 2898, 2899, 2901, 2902, 2909, 2910, 2914, 2916, 2918, 2919, 2920, 2926, 2927, 2932, 2933, 2937, 2938, 2939, 2941, 2942, 2945, 2953, 2954, 2956, 2957, 2960, 2962, 2963, 2964, 2965, 2967, 2972, 2973, 2977, 2979, 2980, 2982, 2983, 2988, 2991, 2993, 2994, 2995, 2997, 3006, 3008, 3012, 3013, 3015, 3023, 3024, 3027, 3028, 3029, 3030, 3031, 3032, 3033, 3034, 3035, 3036, 3037, 3038, 3039, 3043, 3044, 3045, 3046, 3048, 3049, 3050, 3051, 3052, 3053, 3054, 3055, 3059, 3064, 3065, 3071, 3072, 3075, 3076, 3080, 3082, 3084, 3093, 3096, 3098, 3099, 3101, 3119, 3121, 3122, 3123, 3124, 3126, 3128, 3130, 3133, 3142, 3144, 3148, 3159, 3161, 3162, 3163, 3164, 3165, 3166, 3168, 3169, 3170, 3176, 3182, 3188, 3190, 3191, 3195, 3200, 3202, 3203, 3210, 3212, 3216, 3218, 3226, 3227, 3228, 3229, 3230, 3231, 3232, 3234, 3235, 3238, 3239, 3241, 3243, 3244, 3245, 3246, 3247, 3248, 3249, 3250, 3260, 3262, 3263, 3271, 3273, 3275, 3281, 3282, 3283, 3287, 3288, 3289, 3300, 3301, 3302, 3303, 3304, 3305, 3307, 3310, 3311, 3312, 3313, 3314, 3315, 3316, 3317, 3318, 3322, 3324, 3325, 3328, 3330, 3346, 3347, 3348, 3349, 3350, 3358, 3359, 3362, 3364, 3365, 3366, 3367, 3368, 3372, 3373, 3382, 3385, 3387, 3388, 3389, 3390, 3391, 3392, 3393, 3400, 3403, 3404, 3405, 3407, 3408, 3409, 3412, 3414, 3420, 3423, 3425, 3426, 3427, 3428, 3429, 3430, 3431, 3434, 3438, 3441, 3442, 3446, 3449, 3450, 3451, 3452, 3453, 3454, 3455, 3463, 3466, 3469, 3470, 3471, 3472, 3473, 3474, 3477, 3478, 3480, 3481, 3482, 3487, 3490, 3494, 3498, 3499, 3502, 3503, 3505, 3506, 3508, 3509, 3510, 3511, 3513, 3520, 3522, 3523, 3526, 3529, 3533, 3535, 3536, 3542, 3543, 3546, 3547, 3549, 3550, 3553, 3554, 3555, 3557, 3560, 3561, 3563, 3564, 3567, 3568, 3569, 3571, 3574, 3575, 576, 3578, 3579, 3580, 3581, 3583, 3584, 3585, 3592, 3594, 3595, 3596, 3597, 3603, 3612, 3613, 3617, 3622, 3625, 3626, 3627, 3628, 3630, 3631, 3632, 3633, 3635, 3636, 3638, 3639, 3640, 3641, 3642, 3646, 3647, 3648, 3654, 3657, 3659, 3660, 3661, 3664, 3668, 3669, 3673, 3674, 3678, 3679, 3680, 3681, 3684, 3685, 3687, 3688, 3697, 3699, 3702, 3703, 3704, 3705, 3706, 3708, 3710, 3711, 3712, 3714, 3715, 3721, 3722, 3724, 3725, 3728, 3729, 3730, 3731, 3732, 3733, 3734, 3735, 3736, 3737, 3739, 3740, 3741, 3743, 3744, 3746, 3748, 3755, 3761, 3770, 3771, 3773, 3774, 3776, 3778, 3779, 3781, 3782, 3784, 3785, 3792, 3793, 3794, 3795, 3796, 3797, 3798, 3799, 3800, 3801, 3802, 3803, 3804, 3805, 3806, 3807, 3808, 3809, 3810, 3811, 3812, 3814, 3815, 3816, 3820, 3829, 3839, 3841, 3842, 3843, 3844, 3845, 3851, 3852, 3853, 3854, 3855, 3856, 3857, 3858, 3859, 3860, 3861, 3862, 3863, 3864, 3865, 3868, 3869, 3871, 3874, 3875, 3876, 3877, 3878, 3879, 3880, 3882, 3883, 3884, 3885, 3887, 3895, 3899, 3904, 3907, 3908, 3909, 3910, 3911, 3912, 3913, 3914, 3915, 3916, 3917, 3921, 3924, 3927, 3928, 3929, 3931, 3932, 3935, 3936, 3937, 3938, 3939, 3940, 3941, 3942, 3943, 3945, 3946, 3956, 3957, 3961, 3962, 3965, 3971, 3977, 3978, 3979, 3980, 3981, 3982, 3983, 3985, 3988, 3989, 3990, 3991, 3992, 3993, 3994, 3995, 3997, 3998, 3999, 4001, 4002, 4003, 4004, 4009, 4011, 4012, 4013, 4015, 4016, 4017, 4020, 4021, 4023, 4025, 4026, 4028, 4029, 4031, 4032, 4034, 4035, 4036, 4037, 4038, 4040, 4052, 4055, 4056, 4060, 4061, 4066, 4067, 4070, 4077, 4078, 4080, 4081, 4082, 4083, 4084, 4085, 4086, 4087, 4088, 4089, 4090, 4102, 4105, 4106, 4108, 4109, 4110, 4114, 4115, 4117, 4118, 4119, 4128, 4129, 4132, 4136, 4137, 4142, 4147, 4159, 4163, 4168, 4170, 4171, 4172, 4173, 4175, 4182, 4183, 4186, 4188, 4192, 4194, 4199, 4208, 4225, 4226, 4227, 4228, 4232, 4239, 4240, or 4258. Such PEgRNA sequences may further comprise a 3′ motif at the 3′ end of the extension arm, for example, a hairpin-forming motif or a series of 3, 4 or more U nucleotides. Exemplary PEgRNA sequences with such 3′ adaptations include SEQ ID NOs: 2536, 2537, 2552, 2579, 2581, 2583, 2599, 2604, 2622, 2644, 2661, 2662, 2682, 2686, 2691, 2693, 2698, 2710, 2738, 2739, 2745, 2750, 2751, 2771, 2775, 2776, 2782, 2785, 2787, 2818, 2823, 2831, 2835, 2839, 2873, 2874, 2876, 2892, 2895, 2897, 2904, 2906, 2912, 2915, 2917, 2923, 2948, 2952, 2970, 2974, 2987, 2989, 3003, 3022, 3025, 3040, 3079, 3083, 3089, 3100, 3105, 3108, 3120, 3127, 3137, 3149, 3167, 3174, 3184, 3194, 3199, 3225, 3259, 3264, 3267, 3268, 3272, 3276, 3277, 3279, 3286, 3297, 3345, 3355, 3411, 3415, 3424, 3443, 3476, 3501, 3540, 3552, 3650, and 3653. Without being bound by theory, such 3′ motifs are believed to increase PEgRNA stability. Such PEgRNA sequences may alternatively or additionally be adapted for transcription from a DNA template, for example, by including a 5′ terminal G if the spacer of the PEgRNA begins with another nucleotide, by including 6 or 7 U nucleotides at the 3′ end of the extension arm, or both. Exemplary transcription-adapted sequences include SEQ ID NOs: 2625, 2626, 2627, 2639, 2640, 2641, 2642, 2666, 2673, 2679, 2684, 2700, 2703, 2705, 2707, 2709, 2712, 2714, 2718, 2719, 2724, 2731, 2732, 2736, 2737, 2740, 2741, 2742, 2743, 2746, 2754, 2755, 2756, 2763, 2766, 2767, 2778, 2779, 2780, 2781, 2783, 2784, 2803, 2804, 2805, 2806, 2808, 2809, 2813, 2815, 2817, 2819, 2820, 2821, 2822, 2827, 2836, 2837, 2838, 2840, 2845, 2848, 2849, 2850, 2851, 2852, 2853, 2858, 2859, 2860, 2861, 2863, 2865, 2872, 2875, 2877, 2878, 2879, 2880, 2881, 2882, 2883, 2884, 2900, 2903, 2905, 2907, 2908, 2911, 2913, 2921, 2922, 2924, 2925, 2928, 2929, 2930, 2931, 2934, 2935, 2936, 2940, 2943, 2944, 2946, 2947, 2949, 2950, 2951, 2955, 2958, 2959, 2961, 2966, 2968, 2969, 2971, 2975, 2976, 2978, 2981, 2984, 2985, 2986, 2990, 2992, 2996, 2998, 2999, 3000, 3001, 3002, 3004, 3005, 3007, 3009, 3010, 3011, 3014, 3016, 3017, 3018, 3019, 3020, 3021, 3026, 3041, 3042, 3047, 3056, 3057, 3058, 3060, 3061, 3062, 3063, 3066, 3067, 3068, 3069, 3070, 3073, 3074, 3077, 3078, 3081, 3085, 3086, 3087, 3088, 3090, 3091, 3092, 3094, 3095, 3097, 3102, 3103, 3104, 3106, 3107, 3109, 3110, 3111, 3112, 3113, 3114, 3115, 3116, 3117, 3118, 3125, 3129, 3131, 3132, 3134, 3135, 3136, 3138, 3139, 3140, 3141, 3143, 3145, 3146, 3147, 3150, 3151, 3152, 3153, 3154, 3155, 3156, 3157, 3158, 3160, 3171, 3172, 3173, 3175, 3177, 3178, 3179, 3180, 3181, 3183, 3185, 3186, 3187, 3189, 3192, 3193, 3196, 3197, 3198, 3201, 3204, 3205, 3206, 3207, 3208, 3209, 3211, 3213, 3214, 3215, 3217, 3219, 3220, 3221, 3222, 3223, 3224, 3233, 3236, 3237, 3240, 3242, 3251, 3252, 3253, 3254, 3255, 3256, 3257, 3258, 3261, 3265, 3266, 3269, 3270, 3274, 3278, 3280, 3284, 3285, 3290, 3291, 3292, 3293, 3294, 3295, 3296, 3298, 3299, 3306, 3308, 3309, 3319, 3320, 3321, 3323, 3326, 3327, 3329, 3331, 3332, 3333, 3334, 3335, 3336, 3337, 3338, 3339, 3340, 3341, 3342, 3343, 3344, 3351, 3352, 3353, 3354, 3356, 3357, 3360, 3361, 3363, 3369, 3370, 3371, 3374, 3375, 3376, 3377, 3378, 3379, 3380, 3381, 3383, 3384, 3386, 3394, 3395, 3396, 3397, 3398, 3399, 3401, 3402, 3406, 3410, 3413, 3416, 3417, 3418, 3419, 3421, 3422, 3432, 3433, 3435, 3436, 3437, 3439, 3440, 3444, 3445, 3447, 3448, 3456, 3457, 3458, 3459, 3460, 3461, 3462, 3464, 3465, 3467, 3468, 3475, 3479, 3483, 3484, 3485, 3486, 3488, 3489, 3491, 3492, 3493, 3495, 3496, 3497, 3500, 3504, 3507, 3512, 3514, 3515, 3516, 3517, 3518, 3519, 2521, 3524, 3525, 3527, 3528, 3530, 3531, 3532, 3534, 3537, 3538, 3539, 3541, 3544, 3545, 3548, 3551, 3556, 3558, 3559, 3562, 3565, 3566, 3570, 3572, 3573, 3577, 3582, 3586, 3587, 3588, 3589, 3590, 3591, 3593, 3598, 3599, 3600, 3601, 3602, 3604, 3605, 3606, 3607, 3608, 3609, 3610, 3611, 3614, 3615, 3616, 3618, 3619, 3620, 3621, 3623, 3624, 3629, 3634, 3637, 3643, 3644, 3645, 3649, 3651, 3652, 3655, 3656, 3658, 3662, 3663, 3665, 3666, 3667, 3670, 3671, 3672, 3675, 3676, 3677, 3682, 3683, 3686, 3689, 3690, 3691, 3692, 3693, 3694, 3695, 3696, 3698, 3700, 3701, 3707, 3709, 3713, 3716, 3717, 3718, 3719, 3720, 3723, 3726, 3727, 3738, 3742, 3745, 3747, 3749, 3750, 3751, 3752, 3753, 3754, 3756, 3757, 3758, 3759, 3760, 3762, 3763, 3764, 3765, 3766, 3767, 3768, 3769, 3772, 3775, 3777, 3780, 3783, 3786, 3787, 3788, 3789, 3790, 3791, 3813, 3817, 3818, 3819, 3821, 3822, 3823, 3824, 3825, 3826, 3827, 3828, 3830, 3831, 3832, 3833, 3834, 3835, 3836, 3837, 3838, 3840, 3846, 3847, 3848, 3849, 3850, 3866, 3867, 3870, 3872, 3873, 3881, 3886, 3888, 3889, 3890, 3891, 3892, 3893, 3894, 3896, 3897, 3898, 3900, 3901, 3902, 3903, 3905, 3906, 3918, 3919, 3920, 3922, 3923, 3925, 3926, 3930, 3933, 3934, 3944, 3947, 3948, 3949, 3950, 3951, 3952, 3953, 3954, 3955, 3958, 3959, 3960, 3963, 3964, 3966, 3967, 3968, 3969, 3970, 3972, 3973, 3974, 3975, 3976, 3984, 3986, 3987, 3996, 4000, 4005, 4006, 4007, 4008, 4010, 4014, 4018, 4019, 4022, 4024, 4027, 4030, 4033, 4039, 4041, 4042, 4043, 4044, 4045, 4046, 4047, 4048, 4049, 4050, 4051, 4053, 4054, 4057, 4058, 4059, 4062, 4063, 4064, 4065, 4068, 4069, 4071, 4072, 4073, 4074, 4075, 4076, 4079, 4091, 4092, 4093, 4094, 4095, 4096, 4097, 4098, 4099, 4100, 4101, 4103, 4104, 4107, 4111, 4112, 4113, 4116, 4120, 4121, 4122, 4123, 4124, 4125, 4126, 4127, 4130, 4131, 4133, 4134, 4135, 4138, 4139, 4140, 4141, 4143, 4144, 4145, 4146, 4148, 4149, 4150, 4151, 4152, 4153, 4154, 4155, 4156, 4157, 4158, 4160, 4161, 4162, 4164, 4165, 4166, 4167, 4169, 4174, 4176, 4177, 4178, 4179, 4180, 4181, 4184, 4185, 4187, 4189, 4190, 4191, 4193, 4195, 4196, 4197, 4198, 4200, 4201, 4202, 4203, 4204, 4205, 4206, 4207, 4209, 4210, 4211, 4212, 4213, 4214, 4215, 4216, 4217, 4218, 4219, 4220, 4221, 4222, 4223, 4224, 4229, 4230, 4231, 4233, 4234, 4235, 4236, 4237, 4238, 4241, 4242, 4243, 4244, 4245, 4246, 4247, 4248, 4249, 4250, 4251, 4252, 4253, 4254, 4255, 4256, 4257, 4259, 4260, 4261, 4262, 4263, 4264, 4265, 4266, 4267, 4268, 4269, 4270, 4271, 4272, 4273, 4274, 4275, 4276, 4277, 4278, 4279, 4280, 4281, 4282, 4283, 4284, 4285, 4286, 4287, 4288, 4289, 4290, 4291, 4292, 4293, 4294, 4295, 4296, 4297, 4298, 4299, 4300, 4301, 4302, 4303, 4304, 4305, 4306, 4307, 4308, 4309, 4310, 4311, 4312, 4313, 4314, 4315, 4316, 4317, 4318, 4319, 4320, 4321, 4322, 4323, 4324, 4325, 4326, 4327, 4328, 4329, 4330, 4331, 4332, 4333, 4334, 4335, 4336, 4337, 4338, 4339, 4340, 4341, 4342, 4343, 4344, 4345, 4346, 4347, 4348, 4349, 4350, 4351, 4352, 4353, 4354, 4355, 4356, 4357, 4358, 4359, 4360, 4361, 4362, 4363, 4364, 4365, 4366, 4367, 4368, 4369, 4370, 4371, 4372, 4373, 4374, 4375, 4376, 4377, 4378, 4379, 4380, 4381, 4382, 4383, 4384, 4385, 4386, 4387, 4388, 4389, 4390, 4391, 4392, 4393, 4394, 4395, 4396, 4397, 4398, 4399, 4400, 4401, 4402, 4403, 4404, 4405, 4406, 4407, 4408, and 4409. Such plasmid adapted sequences may further comprise a hairpin-forming motif between the PBS and the 3′ terminal U series.

Any of the PEgRNAs of Table 15 can be used in a Prime Editing system further comprising a nick guide RNA (ngRNA). Such ngRNA can comprise a spacer comprising nucleotides 5-20 of SEQ ID NO: 41, 60, 61, 62, 63, 64, 65, 66, 69, 71, 2045, 2046, 2047, 2048, 2049, 2050, 2051, 2052, 2053, 2054, 2055, 2056, 2057, 2058, 2059, 2060, 2061, 2062, 2063, 2064, 2065, 2066, 2067, 2068, 2069, 2070, 2071, 2072, 2073, 2074, 2075, 2076, 2077, 2078, 2079, 2080, 2081, 2082, 2083, 2084, 2085, 2086, 2087, 2088, 2089, 2090, 2091, 2092, 2093, 2094, 2095, 2096, 2423, 2424, 2425, 2426, 2427, 2428, 2429, 2430, 2431, 2432, 2433, 2434, 2435, 2436, 2437, 2438, 2439, 2440, 2441, 2442, 2443, or 2444 and a gRNA core capable of binding to a Cas9 protein. The spacer of the ngRNA can comprise nucleotides 4-20, 3-20, 2-20, or 1-20 of SEQ ID NO: 41, 60, 61, 62, 63, 64, 65, 66, 69, 71, 2045, 2046, 2047, 2048, 2049, 2050, 2051, 2052, 2053, 2054, 2055, 2056, 2057, 2058, 2059, 2060, 2061, 2062, 2063, 2064, 2065, 2066, 2067, 2068, 2069, 2070, 2071, 2072, 2073, 2074, 2075, 2076, 2077, 2078, 2079, 2080, 2081, 2082, 2083, 2084, 2085, 2086, 2087, 2088, 2089, 2090, 2091, 2092, 2093, 2094, 2095, 2096, 2423, 2424, 2425, 2426, 2427, 2428, 2429, 2430, 2431, 2432, 2433, 2434, 2435, 2436, 2437, 2438, 2439, 2440, 2441, 2442, 2443, or 2444. The spacer of the ngRNA can comprise SEQ ID NO: 41, 60, 61, 62, 63, 64, 65, 66, 69, 71, 2045, 2046, 2047, 2048, 2049, 2050, 2051, 2052, 2053, 2054, 2055, 2056, 2057, 2058, 2059, 2060, 2061, 2062, 2063, 2064, 2065, 2066, 2067, 2068, 2069, 2070, 2071, 2072, 2073, 2074, 2075, 2076, 2077, 2078, 2079, 2080, 2081, 2082, 2083, 2084, 2085, 2086, 2087, 2088, 2089, 2090, 2091, 2092, 2093, 2094, 2095, 2096, 2423, 2424, 2425, 2426, 2427, 2428, 2429, 2430, 2431, 2432, 2433, 2434, 2435, 2436, 2437, 2438, 2439, 2440, 2441, 2442, 2443, or 2444. The gRNA core of the ngRNA can comprise SEQ ID NO. 5857-5859. Exemplary ngRNA provided in Table 15 can comprise SEQ ID NO: 2257, 2258, 2259, 2260, 2261, 2262, 2263, 2264, 2265, 2266, 2267, 2268, 2269, 2270, 2271, 2272, 2273, 2274, 2275, 2276, 2277, 2278, 2279, 2280, 2281, 2282, 2283, 2284, 2285, 2286, 2287, 2288, 2289, 4410, 4411, 4412, 4413, 4414, 4415, 4416, 4417, 4418, 4419, 4420, 4421, or 4422. Such ngRNA sequences may further comprise a 3′ motif at the 3′ end of the gRNA core, for example, a series of 3, 4 or more U nucleotides. Exemplary ngRNA sequences with such 3′ adaptations include SEQ ID NOs: 2290, 2291, 2292, 4423, or 4424. Without being bound by theory, such 3′ motifs are believed to increase ngRNA stability.

Table 16 provides Prime Editing guide RNAs (PEgRNAs), which can be used in Prime Editing systems disclosed herein. Such Prime Editing systems can comprise a Cas9 protein capable of recognizing an NGG or NG PAM sequence (e.g., GGG or GG), and a reverse transcriptase. The Prime Editing systems (e.g., PE3 or PE3b systems) can further comprise a nick guide RNA (ngRNA). Such PEgRNAs and Prime Editing systems can be used, for example, to correct an H1069Q mutation in ATP7B.

The PEgRNAs of Table 16 comprise: (a) a spacer comprising nucleotides 5-20 of SEQ ID NO: 4425, (b) a gRNA core capable of binding to a Cas9 protein, and (c) an extension arm comprising: (i) an editing template comprising any one of SEQ ID NOs: 4437-4492, and (ii) a primer binding site (PBS) comprising any one of SEQ ID NOs: 2297, 4426, 4427, 4428, 4429, 4430, 4431, 4432, 4433, 4434, 4435, and 4436. The spacer of the PEgRNA can comprise, for example, nucleotides 4-20, 3-20, 2-20, or 1-20 of SEQ ID NO: 4425. The spacer of the PEgRNA can comprise SEQ ID NO: 4425. The RTT and the PBS can comprise respectively SEQ ID NOs: 4437 and 2297, 4437 and 4426, 4437 and 4427, 4437 and 4428, 4437 and 4429, 4437 and 4430, 4437 and 4431, 4437 and 4432, 4437 and 4433, 4437 and 4434, 4437 and 4435, 4437 and 4436, 4438 and 2297, 4438 and 4426, 4438 and 4427, 4438 and 4428, 4438 and 4429, 4438 and 4430, 4438 and 4431, 4438 and 4432, 4438 and 4433, 4438 and 4434, 4438 and 4435, 4438 and 4436, 4439 and 2297, 4439 and 4426, 4439 and 4427, 4439 and 4428, 4439 and 4429, 4439 and 4430, 4439 and 4431, 4439 and 4432, 4439 and 4433, 4439 and 4434, 4439 and 4435, 4439 and 4436, 4440 and 2297, 4440 and 4426, 4440 and 4427, 4440 and 4428, 4440 and 4429, 4440 and 4430, 4440 and 4431, 4440 and 4432, 4440 and 4433, 4440 and 4434, 4440 and 4435, 4440 and 4436, 4441 and 2297, 4441 and 4426, 4441 and 4427, 4441 and 4428, 4441 and 4429, 4441 and 4430, 4441 and 4431, 4441 and 4432, 4441 and 4433, 4441 and 4434, 4441 and 4435, 4441 and 4436, 4442 and 2297, 4442 and 4426, 4442 and 4427, 4442 and 4428, 4442 and 4429, 4442 and 4430, 4442 and 4431, 4442 and 4432, 4442 and 4433, 4442 and 4434, 4442 and 4435, 4442 and 4436, 4443 and 2297, 4443 and 4426, 4443 and 4427, 4443 and 4428, 4443 and 4429, 4443 and 4430, 4443 and 4431, 4443 and 4432, 4443 and 4433, 4443 and 4434, 4443 and 4435, 4443 and 4436, 4444 and 2297, 4444 and 4426, 4444 and 4427, 4444 and 4428, 4444 and 4429, 4444 and 4430, 4444 and 4431, 4444 and 4432, 4444 and 4433, 4444 and 4434, 4444 and 4435, 4444 and 4436, 4445 and 2297, 4445 and 4426, 4445 and 4427, 4445 and 4428, 4445 and 4429, 4445 and 4430, 4445 and 4431, 4445 and 4432, 4445 and 4433, 4445 and 4434, 4445 and 4435, 4445 and 4436, 4446 and 2297, 4446 and 4426, 4446 and 4427, 4446 and 4428, 4446 and 4429, 44, 46 and 4430, 4446 and 4431, 4446 and 4432, 4446 and 4433, 4446 and 4434, 4446 and 4435, 4446 and 4436, 4447 and 2297, 4447 and 4426, 4447 and 4427, 4447 and 4428, 4447 and 4429, 4447 and 4430, 4447 and 4431, 4447 and 4432, 4447 and 4433, 4447 and 4434, 4447 and 4435, 4447 and 4436, 4448 and 2297, 4448 and 4426, 4448 and 4427, 4448 and 4428, 4448 and 4429, 4448 and 4430, 4448 and 4431, 4448 and 4432, 4448 and 4433, 4448 and 4434, 4448 and 4435, 4448 and 4436, 4449 and 2297, 4449 and 4426, 4449 and 4427, 4449 and 4428, 4449 and 4429, 4449 and 4430, 4449 and 4431, 4449 and 4432, 4449 and 4433, 4449 and 4434, 4449 and 4435, 4449 and 4436, 4450 and 2297, 4450 and 4426, 4450 and 4427, 4450 and 4428, 4450 and 4429, 4450 and 4430, 4450 and 4431, 4450 and 4432, 4450 and 4433, 4450 and 4434, 4450 and 4435, 4450 and 4436, 4451 and 2297, 4451 and 4426, 4451 and 4427, 4451 and 4428, 4451 and 4429, 4451 and 4430, 4451 and 4431, 4451 and 4432, 4451 and 4433, 4451 and 4434, 4451 and 4435, 4451 and 4436, 4452 and 2297, 4452 and 4426, 4452 and 4427, 4452 and 4428, 4452 and 4429, 4452 and 4430, 4452 and 4431, 4452 and 4432, 4452 and 4433, 4452 and 4434, 4452 and 4435, 4452 and 4436, 4453 and 2297, 4453 and 4426, 4453 and 4427, 4453 and 4428, 4453 and 4429, 4453 and 4430, 4453 and 4431, 4453 and 4432, 4453 and 4433, 4453 and 4434, 4453 and 4435, 4453 and 4436, 4454 and 2297, 4454 and 4426, 4454 and 4427, 4454 and 4428, 4454 and 4429, 4454 and 4430, 4454 and 4431, 4454 and 4432, 4454 and 4433, 4454 and 4434, 4454 and 4435, 4454 and 4436, 4455 and 2297, 4455 and 4426, 4455 and 4427, 4455 and 4428, 4455 and 4429, 4455 and 4430, 4455 and 4431, 4455 and 4432, 4455 and 4433, 4455 and 4434, 4455 and 4435, 4455 and 4436, 4456 and 2297, 4456 and 4426, 4456 and 4427, 4456 and 4428, 4456 and 4429, 4456 and 4430, 4456 and 4431, 4456 and 4432, 4456 and 4433, 4456 and 4434, 4456 and 4435, 4456 and 4436, 4457 and 2297, 4457 and 4426, 4457 and 4427, 4457 and 4428, 4457 and 4429, 4457 and 4430, 4457 and 4431, 4457 and 4432, 4457 and 4433, 4457 and 4434, 4457 and 4435, 4457 and 4436, 4458 and 2297, 4458 and 4426, 4458 and 4427, 4458 and 4428, 4458 and 4429, 4458 and 4430, 4458 and 4431, 4458 and 4432, 4458 and 4433, 4458 and 4434, 4458 and 4435, 4458 and 4436, 4459 and 2297, 4459 and 4426, 4459 and 4427, 4459 and 4428, 4459 and 4429, 4459 and 4430, 4459 and 4431, 4459 and 4432, 4459 and 4433, 4459 and 4434, 4459 and 4435, 4459 and 4436, 4460 and 2297, 4460 and 4426, 4460 and 4427, 4460 and 4428, 4460 and 4429, 4460 and 4430, 4460 and 4431, 4460 and 4432, 4460 and 4433, 4460 and 4434, 4460 and 4435, 4460 and 4436, 4461 and 2297, 4461 and 4426, 4461 and 4427, 4461 and 4428, 4461 and 4429, 4461 and 4430, 4461 and 4431, 4461 and 4432, 4461 and 4433, 4461 and 4434, 4461 and 4435, 4461 and 4436, 4462 and 2297, 4462 and 4426, 4462 and 4427, 4462 and 4428, 4462 and 4429, 4462 and 4430, 4462 and 4431, 4462 and 4432, 4462 and 4433, 4462 and 4434, 4462 and 4435, 4462 and 4436, 4463 and 2297, 4463 and 4426, 4463 and 4427, 4463 and 4428, 4463 and 4429, 4463 and 4430, 4463 and 4431, 4463 and 4432, 4463 and 4433, 4463 and 4434, 4463 and 4435, 4463 and 4436, 4464 and 2297, 4464 and 4426, 4464 and 4427, 4464 and 4428, 4464 and 4429, 4464 and 4430, 4464 and 4431, 4464 and 4432, 4464 and 4433, 4464 and 4434, 4464 and 4435, 4464 and 4436, 4465 and 2297, 4465 and 4426, 4465 and 4427, 4465 and 4428, 4465 and 4429, 4465 and 4430, 4465 and 4431, 4465 and 4432, 4465 and 4433, 4465 and 4434, 4465 and 4435, 4465 and 4436, 4466 and 2297, 4466 and 4426, 4466 and 4427, 4466 and 4428, 4466 and 4429, 4466 and 4430, 4466 and 4431, 4466 and 4432, 4466 and 4433, 4466 and 4434, 4466 and 4435, 4466 and 4436, 4467 and 2297, 4467 and 4426, 4467 and 4427, 4467 and 4428, 4467 and 4429, 4467 and 4430, 4467 and 4431, 4467 and 4432, 4467 and 4433, 4467 and 4434, 4467 and 4435, 4467 and 4436, 4468 and 2297, 4468 and 4426, 4468 and 4427, 4468 and 4428, 4468 and 4429, 4468 and 4430, 4468 and 4431, 4468 and 4432, 4468 and 4433, 4468 and 4434, 4468 and 4435, 4468 and 4436, 4469 and 2297, 4469 and 4426, 4469 and 4427, 4469 and 4428, 4469 and 4429, 4469 and 4430, 4469 and 4431, 4469 and 4432, 4469 and 4433, 4469 and 4434, 4469 and 4435, 4469 and 4436, 4470 and 2297, 4470 and 4426, 4470 and 4427, 4470 and 4428, 4470 and 4429, 4470 and 4430, 4470 and 4431, 4470 and 4432, 4470 and 4433, 4470 and 4434, 4470 and 4435, 4470 and 4436, 4471 and 2297, 4471 and 4426, 4471 and 4427, 4471 and 4428, 4471 and 4429, 4471 and 4430, 4471 and 4431, 4471 and 4432, 4471 and 4433, 4471 and 4434, 4471 and 4435, 4471 and 4436, 4472 and 2297, 4472 and 4426, 4472 and 4427, 4472 and 4428, 4472 and 4429, 4472 and 4430, 4472 and 4431, 4472 and 4432, 4472 and 4433, 4472 and 4434, 4472 and 4435, 4472 and 4436, 4473 and 2297, 4473 and 4426, 4473 and 4427, 4473 and 4428, 4473 and 4429, 4473 and 4430, 4473 and 4431, 4473 and 4432, 4473 and 4433, 4473 and 4434, 4473 and 4435, 4473 and 4436, 4474 and 2297, 4474 and 4426, 4474 and 4427, 4474 and 4428, 4474 and 4429, 4474 and 4430, 4474 and 4431, 4474 and 4432, 4474 and 4433, 4474 and 4434, 4474 and 4435, 4474 and 4436, 4475 and 2297, 4475 and 4426, 4475 and 4427, 4475 and 4428, 4475 and 4429, 4475 and 4430, 4475 and 4431, 4475 and 4432, 4475 and 4433, 4475 and 4434, 4475 and 4435, 4475 and 4436, 4476 and 2297, 4476 and 4426, 4476 and 4427, 4476 and 4428, 4476 and 4429, 4476 and 4430, 4476 and 4431, 4476 and 4432, 4476 and 4433, 4476 and 4434, 4476 and 4435, 4476 and 4436, 4477 and 2297, 4477 and 4426, 4477 and 4427, 4477 and 4428, 4477 and 4429, 4477 and 4430, 4477 and 4431, 4477 and 4432, 4477 and 4433, 4477 and 4434, 4477 and 4435, 4477 and 4436, 4478 and 2297, 4478 and 4426, 4478 and 4427, 4478 and 4428, 4478 and 4429, 4478 and 4430, 4478 and 4431, 4478 and 4432, 4478 and 4433, 4478 and 4434, 4478 and 4435, 4478 and 4436, 4479 and 2297, 4479 and 4426, 4479 and 4427, 4479 and 4428, 4479 and 4429, 4479 and 4430, 4479 and 4431, 4479 and 4432, 4479 and 4433, 4479 and 4434, 4479 and 4435, 4479 and 4436, 4480 and 2297, 4480 and 4426, 4480 and 4427, 4480 and 4428, 4480 and 4429, 4480 and 4430, 4480 and 4431, 4480 and 4432, 4480 and 4433, 4480 and 4434, 4480 and 4435, 4480 and 4436, 4481 and 2297, 4481 and 4426, 4481 and 4427, 4481 and 4428, 4481 and 4429, 4481 and 4430, 4481 and 4431, 4481 and 4432, 4481 and 4433, 4481 and 4434, 4481 and 4435, 4481 and 4436, 4482 and 2297, 4482 and 4426, 4482 and 4427, 4482 and 4428, 4482 and 4429, 4482 and 4430, 4482 and 4431, 4482 and 4432, 4482 and 4433, 4482 and 4434, 4482 and 4435, 4482 and 4436, 4483 and 2297, 4483 and 4426, 4483 and 4427, 4483 and 4428, 4483 and 4429, 4483 and 4430, 4483 and 4431, 4483 and 4432, 4483 and 4433, 4483 and 4434, 4483 and 4435, 4483 and 4436, 4484 and 2297, 4484 and 4426, 4484 and 4427, 4484 and 4428, 4484 and 4429, 4484 and 4430, 4484 and 4431, 4484 and 4432, 4484 and 4433, 4484 and 4434, 4484 and 4435, 4484 and 4436, 44185 and 2297, 4485 and 4426, 4485 and 4427, 4485 and 4428, 4485 and 4429, 4485 and 4430, 4485 and 4431, 4485 and 4432, 4485 and 4433, 4485 and 4434, 4485 and 4435, 4485 and 4436, 4486 and 2297, 4486 and 4426, 4486 and 4427, 4486 and 4428, 4486 and 4429, 4486 and 4430, 4486 and 4431, 4486 and 4432, 4486 and 4433, 4486 and 4434, 4486 and 4435, 4486 and 4436, 4487 and 2297, 4487 and 4426, 4487 and 4427, 4487 and 4428, 4487 and 4429, 4487 and 4430, 4487 and 4431, 4487 and 4432, 4487 and 4433, 4487 and 4434, 4487 and 4435, 4487 and 4436, 4488 and 2297, 4488 and 4426, 4488 and 4427, 4488 and 4428, 4488 and 4429, 4488 and 4430, 4488 and 4431, 4488 and 4432, 4488 and 4433, 4488 and 4434, 4488 and 4435, 4488 and 4436, 4489 and 2297, 4489 and 4426, 4489 and 4427, 4489 and 4428, 4489 and 4429, 4489 and 4430, 4489 and 4431, 4489 and 4432, 4489 and 4433, 4489 and 4434, 4489 and 4435, 4489 and 4436, 4490 and 2297, 4490 and 4426, 4490 and 4427, 4490 and 4428, 4490 and 4429, 4490 and 4430, 4490 and 4431, 4490 and 4432, 4490 and 4433, 4490 and 4434, 4490 and 4435, 4490 and 4436, 4491 and 2297, 4491 and 4426, 4491 and 4427, 4491 and 4428, 4491 and 4429, 4491 and 4430, 4491 and 4431, 4491 and 4432, 4491 and 4433, 4491 and 4434, 4491 and 4435, 4491 and 4436, 4492 and 2297, 4492 and 4426, 4492 and 4427, 4492 and 4428, 4492 and 4429, 4492 and 4430, 4492 and 4431, 4492 and 4432, 4492 and 4433, 4492 and 4434, 4492 and 4435, or 4492 and 4436. The gRNA core of the PEgRNA can comprise SEQ ID NO. 5857-5859. Exemplary PEgRNAs provided in Table 16 can comprise SEQ ID NO. 4493, 4494, 4495, 4496, 4497, 4498, 4499, 4500, 4501, 4502, 4503, 4504, 4505, 4506, 4507, 4518, 4509, 4510, 4511, 4512, 4513, 4514, 4515, 4516, 4517, 4518, 4519, 4521, 4522, 4523, 4524, 4525, 4526, 4527, 4528, 4529, 4530, 4531, 4532, 4533, 4535, 4537, 4538, 4539, 4540, 4542, 4543, 4544, 4545, 4546, 4547, 4549, 4550, 4552, 4553, 4555, 4556, 4557, 4558, 4559, 4560, 4561, 4562, 4564, 4566, 4567, 4568, 4570, 4573, 4574, 4575, 4576, 4577, 4578, 4579, 4580, 4581, 4582, 4583, 4584, 4585, 4586, 4587, 4588, 4589, 4591, 4592, 4595, 4596, 4597, 4603, 4604, 4605, 4606, 4612, 4614, 4617, 4618, 4619, 4620, 4621, 4623, 4624, 4625, 4626, 4627, 4629, 4630, 4632, 4634, 4635, 4637, 4638, 4639, 4640, 4646, 4649, 4651, 4653, 4654, 4655, 4656, 4657, 4659, 4660, 4661, 4662, 4666, 4667, 4668, 4670, 4673, 4674, 4675, 4676, 4677, 4679, 4682, 4684, 4689, 4691, 4697, 4698, 4700, 4702, 4705, 4710, 4711, 4714, 4717, 4718, 4719, 4720, 4722, 4724, 4725, 4726, 4737, 4738, 4739, 4740, 4742, 4746, 4748, 4749, 4751, 4755, 4756, 4758, 4759, 4760, 4761, 4763, 4765, 4767, 4768, 4776, 4780, 4784, 4785, 4789, 4792, 4795, 4799, 4801, 4802, 4804, 4809, 4811, 4812, 4816, 4817, 4820, 4822, 4826, 4827, 4828, 4832, 4835, 4836, 4839, 4840, 4841, 4842, 4848, 4852, 4856, 4857, 4859, 4860, 4861, 4862, 4864, 4865, 4868, 4871, 4873, 4874, 4875, 4876, 4882, 4887, 4889, 4893, 4895, 4896, 4898, 4902, 4904, 4905, 4907, 4908, 4909, 4912, 4913, 4915, 4922, 4923, 4924, 4932, 4934, 4935, 4936, 4937, 4939, 4947, 4949, 4950, 4951, 4954, 4955, 4956, 4957, 4958, 4959, 4961, 4963, 4969, 4970, 4971, 4972, 4976, 4978, 4979, 4980, 4985, 4986, 4987, 4990, 4993, 4995, 4998, 5000, 5001, 5002, 5003, 5004, 5005, 5010, 5011, 5018, 5019, 5020, 5021, 5022, 5023, 5024, 5026, 5029, 5030, 5033, 5034, 5035, 5036, 5037, 5039, 5042, 5043, 5044, 5045, 5046, 5047, 5053, 5054, 5056, 5057, 5058, 5060, 5062, 5063, 5066, 5070, 5071, 5072, 5073, 5076, 5077, 5084, 5088, 5091, 5092, 5093, 5094, 5096, 5097, 5098, 5103, 5110, 5111, 5112, 5115, 5119, 5123, 5132, 5135, 5138, 5147, 5151, 5164, 5167, 5175, 5176, 5180, 5181, 5183, 5184, 5185, 5186, 5187, 5188, 5189, 5190, 5191, 5192, 5193, 5194, 5195, 5196, 5197, 5198, 5199, 5200, 5201, 5202, 5203, 5204, or 5205. Such PEgRNA sequences may further comprise a 3′ motif at the 3′ end of the extension arm, for example, a hairpin-forming motif or a series of 3, 4 or more U nucleotides. Exemplary PEgRNA sequences with such 3′ adaptations include SEQ ID NOs: 4520, 4534, 4536, 4541, 4548, 4551, 4554, 4563, 4565, 4571, 4572, 4590, 4593, 4594, 4601, 4602, 4608, 4609, 4610, 4611, 4613, 4622, 4631, 4636, 4641, 4643, 4644, 4647, 4648, 4672, 4680, 4681, 4685, 4686, 4687, 4688, 4693, 4695, 4696, 4703, 4706, 4708, 4709, 4712, 4721, 4723, 4728, 4730, 4733, 4735, 4736, 4741, 4743, 4744, 4747, 4753, 4762, 4769, 4770, 4772, 4778, 4779, 4781, 4782, 4788, 4791, 4793, 4797, 4803, 4805, 4807, 4808, 4818, 4819, 4821, 4823, 4824, 4825, 4829, 4847, 4851, 4854, 4866, 4867, 4872, 4879, 4880, 4885, 4891, 4892, 4900, 4910, 4911, 4919, 4926, 4928, 4946, 4967, and 4983. Without being bound by theory, such 3′ motifs are believed to increase PEgRNA stability. Such PEgRNA sequences may alternatively or additionally be adapted for transcription from a DNA template, for example, by including a 5′ terminal G if the spacer of the PEgRNA begins with another nucleotide, by including 6 or 7 U nucleotides at the 3′ end of the extension arm, or both. Exemplary transcription-adapted sequences include SEQ ID NOs: 4569, 4598, 4599, 4600, 4607, 4615, 4616, 4628, 4633, 4642, 4645, 4650, 4652, 4658, 4663, 4664, 4665, 4669, 4671, 4678, 4683, 4690, 4692, 4694, 4699, 4701, 4704, 4707, 4713, 4715, 4716, 4727, 4729, 4731, 4732, 4734, 4745, 4750, 4752, 4754, 4757, 4764, 4766, 4771, 4773, 4774, 4775, 4777, 4783, 4786, 4787, 4790, 4794, 4796, 4798, 4800, 4806, 4810, 4813, 4814, 4815, 4830, 4831, 4833, 4834, 4837, 4838, 4843, 4844, 4845, 4846, 4849, 4850, 4853, 4855, 4858, 4863, 4869, 4870, 4877, 4878, 4881, 4883, 4884, 4886, 4888, 4890, 4894, 4897, 4899, 4901, 49031, 4906, 4914, 4916, 4917, 4918, 4920, 4921, 4925, 4927, 4929, 4930, 4931, 4933, 4938, 4940, 4941, 4942, 4943, 4944, 4945, 4948, 4952, 4953, 4960, 4962, 4964, 4965, 4966, 4968, 4973, 4974, 4975, 4977, 4981, 4982, 4984, 4988, 4989, 4991, 4992, 4994, 4996, 4997, 4999, 5006, 5007, 5008, 5009, 5012, 5013, 5014, 5015, 5016, 5017, 5025, 5027, 5028, 5031, 5032, 5038, 5040, 5041, 5048, 5049, 5050, 5051, 5052, 5055, 5059, 5061, 5064, 5065, 5067, 5068, 5069, 5074, 5075, 5078, 5079, 5080, 5081, 5082, 5083, 5085, 5086, 5087, 5089, 5090, 5095, 5099, 5100, 5101, 5102, 5104, 5105, 5106, 5107, 5108, 5109, 5113, 5114, 5116, 5117, 5118, 5120, 5121, 5122, 5124, 5125, 5126, 5127, 5128, 5129, 5130, 5131, 5133, 5134, 5136, 5137, 5139, 5140, 5141, 5142, 5143, 5144, 5145, 5146, 5148, 5149, 5150, 5152, 5153, 5154, 5155, 5156, 5157, 5158, 5159, 5160, 5161, 5162, 5163, 5165, 5166, 5168, 5169, 5170, 5171, 5172, 5173, 5174, 5177, 5178, 5179, and 5182. Such plasmid adapted sequences may further comprise a hairpin-forming motif between the PBS and the 3′ terminal U series.

Any of the PEgRNAs of Table 16 can be used in a Prime Editing system further comprising a nick guide RNA (ngRNA). Such ngRNA can comprise a spacer comprising nucleotides 5-20 of SEQ ID NO: 41, 60, 61, 62, 63, 64, 65, 66, 69, 71, 2045, 2046, 2047, 2048, 2049, 2050, 2051, 2052, 2053, 2054, 2055, 2056, 2057, 2058, 2059, 2060, 2061, 2062, 2063, 2064, 2065, 2066, 2067, 2068, 2069, 2070, 2071, 2072, 2073, 2074, 2075, 2076, 2077, 2078, 2079, 2080, 2081, 2082, 2083, 2084, 2085, 2086, 2087, 2088, 2089, 2090, 2091, 2092, 2093, 2094, 2095, 2096, 2437, 2439, 2442, 2443, or 2444 and a gRNA core capable of binding to a Cas9 protein. The spacer of the ngRNA can comprise nucleotides 4-20, 3-20, 2-20, or 1-20 of SEQ ID NO: 41, 60, 61, 62, 63, 64, 65, 66, 69, 71, 2045, 2046, 2047, 2048, 2049, 2050, 2051, 2052, 2053, 2054, 2055, 2056, 2057, 2058, 2059, 2060, 2061, 2062, 2063, 2064, 2065, 2066, 2067, 2068, 2069, 2070, 2071, 2072, 2073, 2074, 2075, 2076, 2077, 2078, 2079, 2080, 2081, 2082, 2083, 2084, 2085, 2086, 2087, 2088, 2089, 2090, 2091, 2092, 2093, 2094, 2095, 2096, 2437, 2439, 2442, 2443, or 2444. The spacer of the ngRNA can comprise SEQ ID NO: 41, 60, 61, 62, 63, 64, 65, 66, 69, 71, 2045, 2046, 2047, 2048, 2049, 2050, 2051, 2052, 2053, 2054, 2055, 2056, 2057, 2058, 2059, 2060, 2061, 2062, 2063, 2064, 2065, 2066, 2067, 2068, 2069, 2070, 2071, 2072, 2073, 2074, 2075, 2076, 2077, 2078, 2079, 2080, 2081, 2082, 2083, 2084, 2085, 2086, 2087, 2088, 2089, 2090, 2091, 2092, 2093, 2094, 2095, 2096, 2437, 2439, 2442, 2443, or 2444. The gRNA core of the ngRNA can comprise SEQ ID NO. 5857-5859. Exemplary ngRNA provided in Table 16 can comprise SEQ ID NO: 2257, 2258, 2259, 2260, 2261, 2262, 2263, 2264, 2265, 2266, 2267, 2268, 2269, 2270, 2271, 2272, 2273, 2274, 2275, 2276, 2277, 2278, 2279, 2280, 2281, 2282, 2283, 2284, 2285, 2286, 2287, 2288, 2289, 4411, 4416, 4418, 4420, or 4422. Such ngRNA sequences may further comprise a 3′ motif at the 3′ end of the gRNA core, for example, a series of 3, 4 or more U nucleotides. Exemplary ngRNA sequences with such 3′ adaptations include SEQ ID NOs: 2290-2292. Without being bound by theory, such 3′ motifs are believed to increase ngRNA stability.

Table 17 provides Prime Editing guide RNAs (PEgRNAs), which can be used in Prime Editing systems disclosed herein. Such Prime Editing systems can comprise a Cas9 protein capable of recognizing an NGA, NGN, NRN, or NG PAM sequence (e.g., GGA or GG), and a reverse transcriptase. The Prime Editing systems (e.g., PE3 or PE3b systems) can further comprise a nick guide RNA (ngRNA). Such PEgRNAs and Prime Editing systems can be used, for example, to correct an H1069Q mutation in ATP7B.

The PEgRNAs of Table 17 comprise: (a) a spacer comprising nucleotides 5-20 of SEQ ID NO: 5206, (b) a gRNA core capable of binding to a Cas9 protein, and (c) an extension arm comprising: (i) an editing template comprising SEQ ID NO: 5218, and (ii) a primer binding site (PBS) comprising any one of SEQ ID NOs: 5207-5217. The spacer of the PEgRNA can comprise, for example, nucleotides 4-20, 3-20, 2-20, or 1-20 of SEQ ID NO: 5206. The spacer of the PEgRNA can comprise SEQ ID NO: 5206. The RTT and the PBS can comprise respectively SEQ ID NOs: 5218 and 5207, 5218 and 5208, 5218 and 5209, 5218 and 5210, 5218 and 5211, 5218 and 5212, 5218 and 5213, 5218 and 5214, 5218 and 5215, 5218 and 5216, or 5218 and 5217. The gRNA core of the PEgRNA can comprise SEQ ID NO. 5857-5859. Exemplary PEgRNAs provided in Table 17 may further comprise a 3′ motif at the 3′ end of the extension arm, for example, a hairpin-forming motif or a series of 3, 4 or more U nucleotides. Without being bound by theory, such 3′ motifs are believed to increase PEgRNA stability. Such PEgRNA sequences may alternatively or additionally be adapted for transcription from a DNA template, for example, by including a 5′ terminal G if the spacer of the PEgRNA begins with another nucleotide, by including 6 or 7 U nucleotides at the 3′ end of the extension arm, or both. Such plasmid adapted sequences may further comprise a hairpin-forming motif between the PBS and the 3′ terminal U series.

Any of the PEgRNAs of Table 17 can be used in a Prime Editing system further comprising a nick guide RNA (ngRNA). Such ngRNA can comprise a spacer comprising nucleotides 5-20 of SEQ ID NO: 18, 19, 20, 21, 22, 23, 24, 25, 26, 27, 28, 29, 30, 31, 32, 33, 34, 35, 36, 37, 38, 39, 40, 42, 43, 44, 45, 46, 47, 48, 49, 50, 51, 52, 53, 54, 55, 56, 57, 58, 59, 67, 68, 70, 72, 199, 200, 203, 529, 737, 5219, 5220, 5221, 5222, 5223, 5224, 5225, 5226, or 5227 and a gRNA core capable of binding to a Cas9 protein. The spacer of the ngRNA can comprise nucleotides 4-20, 3-20, 2-20, or 1-20 of SEQ ID NO: 18, 19, 20, 21, 22, 23, 24, 25, 26, 27, 28, 29, 30, 31, 32, 33, 34, 35, 36, 37, 38, 39, 40, 42, 43, 44, 45, 46, 47, 48, 49, 50, 51, 52, 53, 54, 55, 56, 57, 58, 59, 67, 68, 70, 72, 199, 200, 203, 529, 737, 5219, 5220, 5221, 5222, 5223, 5224, 5225, 5226, or 5227. The spacer of the ngRNA can comprise SEQ ID NO: 18, 19, 20, 21, 22, 23, 24, 25, 26, 27, 28, 29, 30, 31, 32, 33, 34, 35, 36, 37, 38, 39, 40, 42, 43, 44, 45, 46, 47, 48, 49, 50, 51, 52, 53, 54, 55, 56, 57, 58, 59, 67, 68, 70, 72, 199, 200, 203, 529, 737, 5219, 5220, 5221, 5222, 5223, 5224, 5225, 5226, or 5227. The gRNA core of the ngRNA can comprise SEQ ID NO. 5857-5859. Exemplary ngRNA provided in Table 17 can comprise SEQ ID NOs: 153, 154, 155, 156, 157, 158, 159, 160, 161, 162, 163, 164, 165, 166, 167, 168, 169, 170, 171, 172, 173, 174, 175, 176, 177, 178, 179, 180, 181, 290, 291, 292, 293, 681, 1501, or 1504. Such ngRNA sequences may further comprise a 3′ motif at the 3′ end of the gRNA core, for example, a series of 3, 4 or more U nucleotides. Without being bound by theory, such 3′ motifs are believed to increase ngRNA stability.

Table 18 provides Prime Editing guide RNAs (PEgRNAs), which can be used in Prime Editing systems disclosed herein. Such Prime Editing systems can comprise a Cas9 protein capable of recognizing an NGA or NG PAM sequence (e.g., GGA or GG), and a reverse transcriptase. The Prime Editing systems (e.g., PE3 or PE3b systems) can further comprise a nick guide RNA (ngRNA). Such PEgRNAs and Prime Editing systems can be used, for example, to correct an H1069Q mutation in ATP7B.

The PEgRNAs of Table 18 comprise: (a) a spacer comprising nucleotides 5-20 of SEQ ID NO: 5228, (b) a gRNA core capable of binding to a Cas9 protein, and (c) an extension arm comprising: (i) an editing template comprising any one of SEQ ID NOs: 5240-5247, and (ii) a primer binding site (PBS) comprising any one of SEQ ID NOs: 5229-5239. The spacer of the PEgRNA can comprise, for example, nucleotides 4-20, 3-20, 2-20, or 1-20 of SEQ ID NO: 5228. The spacer of the PEgRNA can comprise SEQ ID NO: 5228. The RTT and the PBS can comprise respectively SEQ ID NOs: 5240 and 5229, 5240 and 5230, 5240 and 5231, 5240 and 5232, 5240 and 5233, 5240 and 5234, 5240 and 5235, 5240 and 5236, 5240 and 5237, 5240 and 5238, 5240 and 5239, 5241 and 5229, 5241 and 5230, 5241 and 5231, 5241 and 5232, 5241 and 5233, 5241 and 5234, 5241 and 5235, 5241 and 5236, 5241 and 5237, 5241 and 5238, 5241 and 5239, 5242 and 5229, 5242 and 5230, 5242 and 5231, 5242 and 5232, 5242 and 5233, 5242 and 5234, 5242 and 5235, 5242 and 5236, 5242 and 5237, 5242 and 5238, 5242 and 5239, 5243 and 5229, 5243 and 5230, 5243 and 5231, 5243 and 5232, 5243 and 5233, 5243 and 5234, 5243 and 5235, 5243 and 5236, 5243 and 5237, 5243 and 5238, 5243 and 5239, 5244 and 5229, 5244 and 5230, 5244 and 5231, 5244 and 5232, 5244 and 5233, 5244 and 5234, 5244 and 5235, 5244 and 5236, 5244 and 5237, 5244 and 5238, 5244 and 5239, 5245 and 5229, 5245 and 5230, 5245 and 5231, 5245 and 5232, 5245 and 5233, 5245 and 5234, 5245 and 5235, 5245 and 5236, 5245 and 5237, 5245 and 5238, 5245 and 5239, 5246 and 5229, 5246 and 5230, 5246 and 5231, 5246 and 5232, 5246 and 5233, 5246 and 5234, 5246 and 5235, 5246 and 5236, 5246 and 5237, 5246 and 5238, 5246 and 5239, 5247 and 5229, 5247 and 5230, 5247 and 5231, 5247 and 5232, 5247 and 5233, 5247 and 5234, 5247 and 5235, 5247 and 5236, 5247 and 5237, 5247 and 5238, or 5247 and 5239. The gRNA core of the PEgRNA can comprise SEQ ID NO. 5857-5859. Such PEgRNA sequences may further comprise a 3′ motif at the 3′ end of the extension arm, for example, a hairpin-forming motif or a series of 3, 4 or more U nucleotides. Without being bound by theory, such 3′ motifs are believed to increase PEgRNA stability. Such PEgRNA sequences may alternatively or additionally be adapted for transcription from a DNA template, for example, by including a 5′ terminal G if the spacer of the PEgRNA begins with another nucleotide, by including 6 or 7 U nucleotides at the 3′ end of the extension arm, or both. Such plasmid adapted sequences may further comprise a hairpin-forming motif between the PBS and the 3′ terminal U series.

Any of the PEgRNAs of Table 18 can be used in a Prime Editing system further comprising a nick guide RNA (ngRNA). Such ngRNA can comprise a spacer comprising nucleotides 5-20 of SEQ ID NOs: 18, 19, 20, 21, 22, 23, 24, 25, 26, 27, 28, 29, 30, 31, 32, 33, 34, 35, 36, 37, 38, 39, 40, 42, 43, 44, 45, 46, 47, 48, 49, 50, 51, 52, 53, 54, 55, 56, 57, 58, 59, 67, 68, 70, 72, 199, 200, 203, 529, 736, 737, 738, 739, 740, 5219, 5220, 5221, 5222, 5223, 5224, 5225, 5226, or 5227 and a gRNA core capable of binding to a Cas9 protein. The spacer of the ngRNA can comprise nucleotides 4-20, 3-20, 2-20, or 1-20 of SEQ ID NO: 18, 19, 20, 21, 22, 23, 24, 25, 26, 27, 28, 29, 30, 31, 32, 33, 34, 35, 36, 37, 38, 39, 40, 42, 43, 44, 45, 46, 47, 48, 49, 50, 51, 52, 53, 54, 55, 56, 57, 58, 59, 67, 68, 70, 72, 199, 200, 203, 529, 736, 737, 738, 739, 740, 5219, 5220, 5221, 5222, 5223, 5224, 5225, 5226, or 5227. The spacer of the ngRNA can comprise SEQ ID NO: 18, 19, 20, 21, 22, 23, 24, 25, 26, 27, 28, 29, 30, 31, 32, 33, 34, 35, 36, 37, 38, 39, 40, 42, 43, 44, 45, 46, 47, 48, 49, 50, 51, 52, 53, 54, 55, 56, 57, 58, 59, 67, 68, 70, 72, 199, 200, 203, 529, 736, 737, 738, 739, 740, 5219, 5220, 5221, 5222, 5223, 5224, 5225, 5226, or 5227. The gRNA core of the ngRNA can comprise SEQ ID NO. 5857-5859. Exemplary ngRNA provided in Table 18 can comprise SEQ ID NO: 153, 154, 155, 156, 157, 158, 159, 160, 161, 162, 163, 164, 165, 166, 167, 168, 169, 170, 171, 172, 173, 174, 175, 176, 177, 178, 179, 180, 181, 290, 291, 292, 293, 681, 1501, 1502, 1503, or 1504. Such ngRNA sequences may further comprise a 3′ motif at the 3′ end of the gRNA core, for example, a series of 3, 4 or more U nucleotides. Without being bound by theory, such 3′ motifs are believed to increase ngRNA stability.

Table 19 provides Prime Editing guide RNAs (PEgRNAs), which can be used in Prime Editing systems disclosed herein. Such Prime Editing systems can comprise a Cas9 protein capable of recognizing an NGA or NG PAM sequence (e.g., TGA or TG), and a reverse transcriptase. The Prime Editing systems (e.g., PE3 or PE3b systems) can further comprise a nick guide RNA (ngRNA). Such PEgRNAs and Prime Editing systems can be used, for example, to correct an H1069Q mutation in ATP7B.

The PEgRNAs of Table 19 comprise: (a) a spacer comprising nucleotides 5-20 of SEQ ID NO: 5248, (b) a gRNA core capable of binding to a Cas9 protein, and (c) an extension arm comprising: (i) an editing template comprising any one of SEQ ID NOs: 5260-5279, and (ii) a primer binding site (PBS) comprising any one of SEQ ID NOs: 5249-5259. The spacer of the PEgRNA can comprise, for example, nucleotides 4-20, 3-20, 2-20, or 1-20 of SEQ ID NO: 5248. The spacer of the PEgRNA can comprise SEQ ID NO: 5248. The RTT and the PBS can comprise respectively SEQ ID NOs: 5260 and 5249, 5260 and 5250, 5260 and 5251, 5260 and 5252, 5260 and 5253, 5260 and 5254, 5260 and 5255, 5260 and 5256, 5260 and 5257, 5260 and 5258, 5260 and 5259, 5261 and 5249, 5261 and 5250, 5261 and 5251, 5261 and 5252, 5261 and 5253, 5261 and 5254, 5261 and 5255, 5261 and 5256, 5261 and 5257, 5261 and 5258, 5261 and 5259, 5262 and 5249, 5262 and 5250, 5262 and 5251, 5262 and 5252, 5262 and 5253, 5262 and 5254, 5262 and 5255, 5262 and 5256, 5262 and 5257, 5262 and 5258, 5262 and 5259, 5263 and 5249, 5263 and 5250, 5263 and 5251, 5263 and 5252, 5263 and 5253, 5263 and 5254, 5263 and 5255, 5263 and 5256, 5263 and 5257, 5263 and 5258, 5263 and 5259, 5264 and 5249, 5264 and 5250, 5264 and 5251, 5264 and 5252, 5264 and 5253, 5264 and 5254, 5264 and 5255, 5264 and 5256, 5264 and 5257, 5264 and 5258, 5264 and 5259, 5265 and 5249, 5265 and 5250, 5265 and 5251, 5265 and 5252, 5265 and 5253, 5265 and 5254, 5265 and 5255, 5265 and 5256, 5265 and 5257, 5265 and 5258, 5265 and 5259, 5266 and 5249, 5266 and 5250, 5266 and 5251, 5266 and 5252, 5266 and 5253, 5266 and 5254, 5266 and 5255, 5266 and 5256, 5266 and 5257, 5266 and 5258, 5266 and 5259, 5267 and 5249, 5267 and 5250, 5267 and 5251, 5267 and 5252, 5267 and 5253, 5267 and 5254, 5267 and 5255, 5267 and 5256, 5267 and 5257, 5267 and 5258, 5267 and 5259, 5268 and 5249, 5268 and 5250, 5268 and 5251, 5268 and 5252, 5268 and 5253, 5268 and 5254, 5268 and 5255, 5268 and 5256, 5268 and 5257, 5268 and 5258, 5268 and 5259, 5269 and 5249, 5269 and 5250, 5269 and 5251, 5269 and 5252, 5269 and 5253, 5269 and 5254, 5269 and 5255, 5269 and 5256, 5269 and 5257, 5269 and 5258, 5269 and 5259, 5270 and 5249, 5270 and 5250, 5270 and 5251, 5270 and 5252, 5270 and 5253, 5270 and 5254, 5270 and 5255, 5270 and 5256, 5270 and 5257, 5270 and 5258, 5270 and 5259, 5271 and 5249, 5271 and 5250, 5271 and 5251, 5271 and 5252, 5271 and 5253, 5271 and 5254, 5271 and 5255, 5271 and 5256, 5271 and 5257, 5271 and 5258, 5271 and 5259, 5272 and 5249, 5272 and 5250, 5272 and 5251, 5272 and 5252, 5272 and 5253, 5272 and 5254, 5272 and 5255, 5272 and 5256, 5272 and 5257, 5272 and 5258, 5272 and 5259, 5273 and 5249, 5273 and 5250, 5273 and 5251, 5273 and 5252, 5273 and 5253, 5273 and 5254, 5273 and 5255, 5273 and 5256, 5273 and 5257, 5273 and 5258, 5273 and 5259, 5274 and 5249, 5274 and 5250, 5274 and 5251, 5274 and 5252, 5274 and 5253, 5274 and 5254, 5274 and 5255, 5274 and 5256, 5274 and 5257, 5274 and 5258, 5274 and 5259, 5275 and 5249, 5275 and 5250, 5275 and 5251, 5275 and 5252, 5275 and 5253, 5275 and 5254, 5275 and 5255, 5275 and 5256, 5275 and 5257, 5275 and 5258, 5275 and 5259, 5276 and 5249, 5276 and 5250, 5276 and 5251, 5276 and 5252, 5276 and 5253, 5276 and 5254, 5276 and 5255, 5276 and 5256, 5276 and 5257, 5276 and 5258, 5276 and 5259, 5277 and 5249, 5277 and 5250, 5277 and 5251, 5277 and 5252, 5277 and 5253, 5277 and 5254, 5277 and 5255, 5277 and 5256, 5277 and 5257, 5277 and 5258, 5277 and 5259, 5278 and 5249, 5278 and 5250, 5278 and 5251, 5278 and 5252, 5278 and 5253, 5278 and 5254, 5278 and 5255, 5278 and 5256, 5278 and 5257, 5278 and 5258, 5278 and 5259, 5279 and 5249, 5279 and 5250, 5279 and 5251, 5279 and 5252, 5279 and 5253, 5279 and 5254, 5279 and 5255, 5279 and 5256, 5279 and 5257, 5279 and 5258, or 5279 and 5259. The gRNA core of the PEgRNA can comprise SEQ ID NO. 5857-5859. Such PEgRNA sequences may further comprise a 3′ motif at the 3′ end of the extension arm, for example, a hairpin-forming motif or a series of 3, 4 or more U nucleotides. Without being bound by theory, such 3′ motifs are believed to increase PEgRNA stability. Such PEgRNA sequences may alternatively or additionally be adapted for transcription from a DNA template, for example, by including a 5′ terminal G if the spacer of the PEgRNA begins with another nucleotide, by including 6 or 7 U nucleotides at the 3′ end of the extension arm, or both. Such plasmid adapted sequences may further comprise a hairpin-forming motif between the PBS and the 3′ terminal U series.

Any of the PEgRNAs of Table 19 can be used in a Prime Editing system further comprising a nick guide RNA (ngRNA). Such ngRNA can comprise a spacer comprising nucleotides 5-20 of SEQ ID NO: 18, 19, 20, 21, 22, 23, 24, 25, 26, 27, 28, 29, 30, 31, 32, 33, 34, 35, 36, 37, 38, 39, 40, 42, 43, 44, 45, 46, 47, 48, 49, 50, 51, 52, 53, 54, 55, 56, 57, 58, 59, 67, 68, 70, 72, 199, 200, 203, 529, 736, 737, 738, 739, 740, 5219, 5220, 5221, 5222, 5223, 5224, 5225, 5226, 5227, 5280, or 5281 and a gRNA core capable of binding to a Cas9 protein. The spacer of the ngRNA can comprise nucleotides 4-20, 3-20, 2-20, or 1-20 of SEQ ID NO: 18, 19, 20, 21, 22, 23, 24, 25, 26, 27, 28, 29, 30, 31, 32, 33, 34, 35, 36, 37, 38, 39, 40, 42, 43, 44, 45, 46, 47, 48, 49, 50, 51, 52, 53, 54, 55, 56, 57, 58, 59, 67, 68, 70, 72, 199, 200, 203, 529, 736, 737, 738, 739, 740, 5219, 5220, 5221, 5222, 5223, 5224, 5225, 5226, 5227, 5280 or 5281. The spacer of the ngRNA can comprise SEQ ID NO: 18, 19, 20, 21, 22, 23, 24, 25, 26, 27, 28, 29, 30, 31, 32, 33, 34, 35, 36, 37, 38, 39, 40, 42, 43, 44, 45, 46, 47, 48, 49, 50, 51, 52, 53, 54, 55, 56, 57, 58, 59, 67, 68, 70, 72, 199, 200, 203, 529, 736, 737, 738, 739, 740, 5219, 5220, 5221, 5222, 5223, 5224, 5225, 5226, 5227, 5280, or 5281. The gRNA core of the ngRNA can comprise SEQ ID NO. 5857-5859. Exemplary ngRNA provided in Table 19 can comprise SEQ ID NOs: 153, 154, 155, 156, 157, 158, 159, 160, 161, 162, 163, 164, 165, 166, 167, 168, 169, 170, 171, 172, 173, 174, 175, 176, 177, 178, 179, 180, 181, 290, 291, 292, 293, 681, 1501, 1502, 1503, or 1504. Such ngRNA sequences may further comprise a 3′ motif at the 3′ end of the gRNA core, for example, a series of 3, 4 or more U nucleotides. Without being bound by theory, such 3′ motifs are believed to increase ngRNA stability.

Table 20 provides Prime Editing guide RNAs (PEgRNAs), which can be used in Prime Editing systems disclosed herein. Such Prime Editing systems can comprise a Cas9 protein capable of recogiizing an NRN or NNGG PAM sequence (e.g., GAG or GAGG), and a reverse transcriptase. The Prime Editing systems (e.g., PE3 or PE3b systems) can further comprise a nick guide RNA (ngRNA). Such PEgRNAs and Prime Editing systems can be used, for example, to correct an H1069Q mutation in ATP7B.

The PEgRNAs of Table 20 comprise: (a) a spacer comprising nucleotides 5-20 of SEQ ID NO: 5282, (b) a gRNA core capable of binding to a Cas9 protein, and (c) an extension arm comprising: (i) an editing template comprising any one of SEQ ID NOs: 5294-5302, and (ii) a primer binding site (PBS) comprising any one of SEQ ID NOs: 5283-5293. The spacer of the PEgRNA can comprise, for example, nucleotides 4-20, 3-20, 2-20, or 1-20 of SEQ ID NO: 5282. The spacer of the PEgRNA can comprise SEQ ID NO: 5282. The RTT and the PBS can comprise respectively SEQ ID NOs: 5294 and 5283, 5294 and 5284, 5294 and 5285, 5294 and 5286, 5294 and 5287, 5294 and 5288, 5294 and 5289, 5294 and 5290, 5294 and 5291, 5294 and 5292, 5294 and 5293, 5295 and 5283, 5295 and 5284, 5295 and 5285, 5295 and 5286, 5295 and 5287, 5295 and 5288, 5295 and 5289, 5295 and 5290, 5295 and 5291, 5295 and 5292, 5295 and 5293, 5296 and 5283, 5296 and 5284, 5296 and 5285, 5296 and 5286, 5296 and 5287, 5296 and 5288, 5296 and 5289, 5296 and 5290, 5296 and 5291, 5296 and 5292, 5296 and 5293, 5297 and 5283, 5297 and 5284, 5297 and 5285, 5297 and 5286, 5297 and 5287, 5297 and 5288, 5297 and 5289, 5297 and 5290, 5297 and 5291, 5297 and 5292, 5297 and 5293, 5298 and 5283, 5298 and 5284, 5298 and 5285, 5298 and 5286, 5298 and 5287, 5298 and 5288, 5298 and 5289, 5298 and 5290, 5298 and 5291, 5298 and 5292, 5298 and 5293, 5299 and 5283, 5299 and 5284, 5299 and 5285, 5299 and 5286, 5299 and 5287, 5299 and 5288, 5299 and 5289, 5299 and 5290, 5299 and 5291, 5299 and 5292, 5299 and 5293, 5300 and 5283, 5300 and 5284, 5300 and 5285, 5300 and 5286, 5300 and 5287, 5300 and 5288, 5300 and 5289, 5300 and 5290, 5300 and 5291, 5300 and 5292, 5300 and 5293, 5301 and 5283, 5301 and 5284, 5301 and 5285, 5301 and 5286, 5301 and 5287, 5301 and 5288, 5301 and 5289, 5301 and 5290, 5301 and 5291, 5301 and 5292, 5301 and 5293, 5302 and 5283, 5302 and 5284, 5302 and 5285, 5302 and 5286, 5302 and 5287, 5302 and 5288, 5302 and 5289, 5302 and 5290, 5302 and 5291, 5302 and 5292, or 5302 and 5293. The gRNA core of the PEgRNA can comprise SEQ ID NO. 5857-5859. Such PEgRNA sequences may further comprise a 3′ motif at the 3′ end of the extension arm, for example, a hairpin-forming motif or a series of 3, 4 or more U nucleotides. Without being bound by theory, such 3′ motifs are believed to increase PEgRNA stability. Such PEgRNA sequences may alternatively or additionally be adapted for transcription from a DNA template, for example, by including a 5′ terminal G if the spacer of the PEgRNA begins with another nucleotide, by including 6 or 7 U nucleotides at the 3′ end of the extension arm, or both. Such plasmid adapted sequences may further comprise a hairpin-forming motif between the PBS and the 3′ terminal U series.

Any of the PEgRNAs of Table 20 can be used in a Prime Editing system further comprising a nick guide RNA (ngRNA). Such ngRNA can comprise a spacer comprising nucleotides 5-20 of SEQ ID NO: 27, 30, 33, 35, 50, 68, 70, 200, 201, 202, 204, 205, 206, 207, 208, 209, 337, 736, 740, 5303, 5304, 5305, 5306, 5307, 5308, 5309, 5310, 5311, or 5312 and a gRNA core capable of binding to a Cas9 protein. The spacer of the ngRNA can comprise nucleotides 4-20, 3-20, 2-20, or 1-20 of SEQ ID NO: 27, 30, 33, 35, 50, 68, 70, 200, 201, 202, 204, 205, 206, 207, 208, 209, 337, 736, 740, 5303, 5304, 5305, 5306, 5307, 5308, 5309, 5310, 5311, or 5312. The spacer of the ngRNA can comprise SEQ ID NO: 27, 30, 33, 35, 50, 68, 70, 200, 201, 202, 204, 205, 206, 207, 208, 209, 337, 736, 740, 5303, 5304, 5305, 5306, 5307, 5308, 5309, 5310, 5311, or 5312. The gRNA core of the ngRNA can comprise SEQ ID NO. 5857-5859. Exemplary ngRNA provided in Table 20 can comprise SEQ ID NOs: 153, 154, 155, 157, 160, 162, 163, 165, 166, 167, 170, 171, 172, 173, 177, 178, 180, 181, 291, 292, 1501, 1503, or 1504. Such ngRNA sequences may further comprise a 3′ motif at the 3′ end of the gRNA core, for example, a series of 3, 4 or more U nucleotides. Without being bound by theory, such 3′ motifs are believed to increase ngRNA stability.

Table 21 provides Prime Editing guide RNAs (PEgRNAs), which can be used in Prime Editing systems disclosed herein. Such Prime Editing systems can comprise a Cas9 protein capable of recognizing an NRN PAM sequence (e.g., CAG), and a reverse transcriptase. The Prime Editing systems (e.g., PE3 or PE3b systems) can further comprise a nick guide RNA (ngRNA). Such PEgRNAs and Prime Editing systems can be used, for example, to correct an H1069Q mutation in ATP7B.

The PEgRNAs of Table 21 comprise: (a) a spacer comprising nucleotides 5-20 of SEQ ID NO: 5313, (b) a gRNA core capable of binding to a Cas9 protein, and (c) an extension arm comprising: (i) an editing template comprising any one of SEQ ID NOs: 5325-5338, and (ii) a primer binding site (PBS) comprising any one of SEQ ID NOs: 5314-5324. The spacer of the PEgRNA can comprise, for example, nucleotides 4-20, 3-20, 2-20, or 1-20 of SEQ ID NO: 5313. The spacer of the PEgRNA can comprise SEQ ID NO: 5313. The RTT and the PBS can comprise respectively SEQ ID NOs: 5325 and 5314, 5325 and 5315, 5325 and 5316, 5325 and 5317, 5325 and 5318, 5325 and 5319, 5325 and 5320, 5325 and 5321, 5325 and 5322, 5325 and 5323, 5325 and 5324, 5326 and 5314, 5326 and 5315, 5326 and 5316, 5326 and 5317, 5326 and 5318, 5326 and 5319, 5326 and 5320, 5326 and 5321, 5326 and 5322, 5326 and 5323, 5326 and 5324, 5327 and 5314, 5327 and 5315, 5327 and 5316, 5327 and 5317, 5327 and 5318, 5327 and 5319, 5327 and 5320, 5327 and 5321, 5327 and 5322, 5327 and 5323, 5327 and 5324, 5328 and 5314, 5328 and 5315, 5328 and 5316, 5328 and 5317, 5328 and 5318, 5328 and 5319, 5328 and 5320, 5328 and 5321, 5328 and 5322, 5328 and 5323, 5328 and 5324, 5329 and 5314, 5329 and 5315, 5329 and 5316, 5329 and 5317, 5329 and 5318, 5329 and 5319, 5329 and 5320, 5329 and 5321, 5329 and 5322, 5329 and 5323, 5329 and 5324, 5330 and 5314, 5330 and 5315, 5330 and 5316, 5330 and 5317, 5330 and 5318, 5330 and 5319, 5330 and 5320, 5330 and 5321, 5330 and 5322, 5330 and 5323, 5330 and 5324, 5331 and 5314, 5331 and 5315, 5331 and 5316, 5331 and 5317, 5331 and 5318, 5331 and 5319, 5331 and 5320, 5331 and 5321, 5331 and 5322, 5331 and 5323, 5331 and 5324, 5332 and 5314, 5332 and 5315, 5332 and 5316, 5332 and 5317, 5332 and 5318, 5332 and 5319, 5332 and 5320, 5332 and 5321, 5332 and 5322, 5332 and 5323, 5332 and 5324, 5333 and 5314, 5333 and 5315, 5333 and 5316, 5333 and 5317, 5333 and 5318, 5333 and 5319, 5333 and 5320, 5333 and 5321, 5333 and 5322, 5333 and 5323, 5333 and 5324, 5334 and 5314, 5334 and 5315, 5334 and 5316, 5334 and 5317, 5334 and 5318, 5334 and 5319, 5334 and 5320, 5334 and 5321, 5334 and 5322, 5334 and 5323, 5334 and 5324, 5335 and 5314, 5335 and 5315, 5335 and 5316, 5335 and 5317, 5335 and 5318, 5335 and 5319, 5335 and 5320, 5335 and 5321, 5335 and 5322, 5335 and 5323, 5335 and 5324, 5336 and 5314, 5336 and 5315, 5336 and 5316, 5336 and 5317, 5336 and 5318, 5336 and 5319, 5336 and 5320, 5336 and 5321, 5336 and 5322, 5336 and 5323, 5336 and 5324, 5337 and 5314, 5337 and 5315, 5337 and 5316, 5337 and 5317, 5337 and 5318, 5337 and 5319, 5337 and 5320, 5337 and 5321, 5337 and 5322, 5337 and 5323, 5337 and 5324, 5338 and 5314, 5338 and 5315, 5338 and 5316, 5338 and 5317, 5338 and 5318, 5338 and 5319, 5338 and 5320, 5338 and 5321, 5338 and 5322, 5338 and 5323, or 5338 and 5324. The gRNA core of the PEgRNA can comprise SEQ ID NO. 5857-5859. Such PEgRNA sequences may further comprise a 3′ motif at the 3′ end of the extension arm, for example, a hairpin-forming motif or a series of 3, 4 or more U nucleotides. Without being bound by theory, such 3′ motifs are believed to increase PEgRNA stability. Such PEgRNA sequences may alternatively or additionally be adapted for transcription from a DNA template, for example, by including a 5′ terminal G if the spacer of the PEgRNA begins with another nucleotide, by including 6 or 7 U nucleotides at the 3′ end of the extension arm, or both. Such plasmid adapted sequences may further comprise a hairpin-forming motif between the PBS and the 3′ terminal U series.

Any of the PEgRNAs of Table 21 can be used in a Prime Editing system further comprising a nick guide RNA (ngRNA). Such ngRNA can comprise a spacer comprising nucleotides 5-20 of SEQ ID NO: 201, 206, 207, 208, 337, 5303, 5305, 5307, 5308, 5309, 5310, 5311, 5312, or 5339 and a gRNA core capable of binding to a Cas9 protein. The spacer of the ngRNA can comprise nucleotides 4-20, 3-20, 2-20, or 1-20 of SEQ ID NO: 201, 206, 207, 208, 337, 5303, 5305, 5307, 5308, 5309, 5310, 5311, 5312, or 5339. The spacer of the ngRNA can comprise SEQ ID NO: 201, 206, 207, 208, 337, 5303, 5305, 5307, 5308, 5309, 5310, 5311, 5312, or 5339. The gRNA core of the ngRNA can comprise SEQ ID NO. 5857-5859. Exemplary ngRNA provided in Table 21 can comprise SEQ ID NO: 155, 160, 165, 166, 171, 172, 177, 291, or 292. Such ngRNA sequences may further comprise a 3′ motif at the 3′ end of the gRNA core, for example, a series of 3, 4 or more U nucleotides. Without being bound by theory, such 3′ motifs are believed to increase ngRNA stability.

Table 22 provides Prime Editing guide RNAs (PEgRNAs), which can be used in Prime Editing systems disclosed herein. Such Prime Editing systems can comprise a Cas9 protein capable of recognizing an NRN PAM sequence (e.g., CAG), and a reverse transcriptase. The Prime Editing systems (e.g., PE3 or PE3b systems) can further comprise a nick guide RNA (ngRNA). Such PEgRNAs and Prime Editing systems can be used, for example, to correct an H1069Q mutation in ATP7B.

The PEgRNAs of Table 22 comprise: (a) a spacer comprising nucleotides 5-20 of SEQ ID NO: 5340, (b) a gRNA core capable of binding to a Cas9 protein, and (c) an extension arm comprising: (i) an editing template comprising any one of SEQ ID NOs: 5352-5368, and (ii) a primer binding site (PBS) comprising any one of SEQ ID NOs: 5341-5351. The spacer of the PEgRNA can comprise, for example, nucleotides 4-20, 3-20, 2-20, or 1-20 of SEQ ID NO: 5340. The spacer of the PEgRNA can comprise SEQ ID NO: 5340. The RTT and the PBS can comprise respectively SEQ ID NOs: 5352 and 5341, 5352 and 5342, 5352 and 5343, 5352 and 5344, 5352 and 5345, 5352 and 5346, 5352 and 5347, 5352 and 5348, 5352 and 5349, 5352 and 5350, 5352 and 5351, 5353 and 5341, 5353 and 5342, 5353 and 5343, 5353 and 5344, 5353 and 5345, 5353 and 5346, 5353 and 5347, 5353 and 5348, 5353 and 5349, 5353 and 5350, 5353 and 5351, 5354 and 5341, 5354 and 5342, 5354 and 5343, 5354 and 5344, 5354 and 5345, 5354 and 5346, 5354 and 5347, 5354 and 5348, 5354 and 5349, 5354 and 5350, 5354 and 5351, 5355 and 5341, 5355 and 5342, 5355 and 5343, 5355 and 5344, 5355 and 5345, 5355 and 5346, 5355 and 5347, 5355 and 5348, 5355 and 5349, 5355 and 5350, 5355 and 5351, 5356 and 5341, 5356 and 5342, 5356 and 5343, 5356 and 5344, 5356 and 5345, 5356 and 5346, 5356 and 5347, 5356 and 5348, 5356 and 5349, 5356 and 5350, 5356 and 5351, 5357 and 5341, 5357 and 5342, 5357 and 5343, 5357 and 5344, 5357 and 5345, 5357 and 5346, 5357 and 5347, 5357 and 5348, 5357 and 5349, 5357 and 5350, 5357 and 5351, 5358 and 5341, 5358 and 5342, 5358 and 5343, 5358 and 5344, 5358 and 5345, 5358 and 5346, 5358 and 5347, 5358 and 5348, 5358 and 5349, 5358 and 5350, 5358 and 5351, 5359 and 5341, 5359 and 5342, 5359 and 5343, 5359 and 5344, 5359 and 5345, 5359 and 5346, 5359 and 5347, 5359 and 5348, 5359 and 5349, 5359 and 5350, 5359 and 5351, 5360 and 5341, 5360 and 5342, 5360 and 5343, 5360 and 5344, 5360 and 5345, 5360 and 5346, 5360 and 5347, 5360 and 5348, 5360 and 5349, 5360 and 5350, 5360 and 5351, 5361 and 5341, 5361 and 5342, 5361 and 5343, 5361 and 5344, 5361 and 5345, 5361 and 5346, 5361 and 5347, 5361 and 5348, 5361 and 5349, 5361 and 5350, 5361 and 5351, 5362 and 5341, 5362 and 5342, 5362 and 5343, 5362 and 5344, 5362 and 5345, 5362 and 5346, 5362 and 5347, 5362 and 5348, 5362 and 5349, 5362 and 5350, 5362 and 5351, 5363 and 5341, 5363 and 5342, 5363 and 5343, 5363 and 5344, 5363 and 5345, 5363 and 5346, 5363 and 5347, 5363 and 5348, 5363 and 5349, 5363 and 5350, 5363 and 5351, 5364 and 5341, 5364 and 5342, 5364 and 5343, 5364 and 5344, 5364 and 5345, 5364 and 5346, 5364 and 5347, 5364 and 5348, 5364 and 5349, 5364 and 5350, 5364 and 5351, 5365 and 5341, 5365 and 5342, 5365 and 5343, 5365 and 5344, 5365 and 5345, 5365 and 5346, 5365 and 5347, 5365 and 5348, 5365 and 5349, 5365 and 5350, 5365 and 5351, 5366 and 5341, 5366 and 5342, 5366 and 5343, 5366 and 5344, 5366 and 5345, 5366 and 5346, 5366 and 5347, 5366 and 5348, 5366 and 5349, 5366 and 5350, 5366 and 5351, 5367 and 5341, 5367 and 5342, 5367 and 5343, 5367 and 5344, 5367 and 5345, 5367 and 5346, 5367 and 5347, 5367 and 5348, 5367 and 5349, 5367 and 5350, 5367 and 5351, 5368 and 5341, 5368 and 5342, 5368 and 5343, 5368 and 5344, 5368 and 5345, 5368 and 5346, 5368 and 5347, 5368 and 5348, 5368 and 5349, 5368 and 5350, or 5368 and 5351. The gRNA core of the PEgRNA can comprise SEQ ID NO. 5857-5859. Such PEgRNA sequences may further comprise a 3′ motif at the 3′ end of the extension arm, for example, a hairpin-forming motif or a series of 3, 4 or more U nucleotides. Without being bound by theory, such 3′ motifs are believed to increase PEgRNA stability. Such PEgRNA sequences may alternatively or additionally be adapted for transcription from a DNA template, for example, by including a 5′ terminal G if the spacer of the PEgRNA begins with another nucleotide, by including 6 or 7 U nucleotides at the 3′ end of the extension arm, or both. Such plasmid adapted sequences may further comprise a hairpin-forming motif between the PBS and the 3′ terminal U series.

Any of the PEgRNAs of Table 22 can be used in a Prime Editing system further comprising a nick guide RNA (ngRNA). Such ngRNA can comprise a spacer comprising nucleotides 5-20 of SEQ ID NO: 201, 206, 207, 208, 337, 5303, 5305, 5307, 5308, 5309, 5310, 5311, 5312, or 5339 and a gRNA core capable of binding to a Cas9 protein. The spacer of the ngRNA can comprise nucleotides 4-20, 3-20, 2-20, or 1-20 of SEQ ID NO: 201, 206, 207, 208, 337, 5303, 5305, 5307, 5308, 5309, 5310, 5311, 5312, or 5339. The spacer of the ngRNA can comprise SEQ ID NO: 201, 206, 207, 208, 337, 5303, 5305, 5307, 5308, 5309, 5310, 5311, 5312, or 5339. The gRNA core of the ngRNA can comprise SEQ ID NO. 5857-5859. Exemplary ngRNA provided in Table 22 can comprise SEQ ID NO: 155, 160, 165, 166, 171, 172, 177, 291, or 292. Such ngRNA sequences may further comprise a 3′ motif at the 3′ end of the gRNA core, for example, a series of 3, 4 or more U nucleotides. Without being bound by theory, such 3′ motifs are believed to increase ngRNA stability.

Table 23 provides Prime Editing guide RNAs (PEgRNAs), which can be used in Prime Editing systems disclosed herein. Such Prime Editing systems can comprise a Cas9 protein capable of recognizing an NRN or NNGG PAM sequence (e.g., AAG or AAGG), and a reverse transcriptase. The Prime Editing systems (e.g., PE3 or PE3b systems) can further comprise a nick guide RNA (ngRNA). Such PEgRNAs and Prime Editing systems can be used, for example, to correct an H1069Q mutation in ATP7B.

The PEgRNAs of Table 23 comprise: (a) a spacer comprising nucleotides 5-20 of SEQ ID NO: 5369, (b) a gRNA core capable of binding to a Cas9 protein, and (c) an extension arm comprising: (i) an editing template comprising any one of SEQ ID NOs: 5381-5401, and (ii) a primer binding site (PBS) comprising any one of SEQ ID NOs: 5370-5380. The spacer of the PEgRNA can comprise, for example, nucleotides 4-20, 3-20, 2-20, or 1-20 of SEQ ID NO: 5369. The spacer of the PEgRNA can comprise SEQ ID NO: 5369. The RTT and the PBS can comprise respectively SEQ ID NOs: 5381 and 5370, 5381 and 5371, 5381 and 5372, 5381 and 5373, 5381 and 5374, 5381 and 5375, 5381 and 5376, 5381 and 5377, 5381 and 5378, 5381 and 5379, 5381 and 5380, 5382 and 5370, 5382 and 5371, 5382 and 5372, 5382 and 5373, 5382 and 5374, 5382 and 5375, 5382 and 5376, 5382 and 5377, 5382 and 5378, 5382 and 5379, 5382 and 5380, 5383 and 5370, 5383 and 5371, 5383 and 5372, 5383 and 5373, 5383 and 5374, 5383 and 5375, 5383 and 5376, 5383 and 5377, 5383 and 5378, 5383 and 5379, 5383 and 5380, 5384 and 5370, 5384 and 5371, 5384 and 5372, 5384 and 5373, 5384 and 5374, 5384 and 5375, 5384 and 5376, 5384 and 5377, 5384 and 5378, 5384 and 5379, 5384 and 5380, 5385 and 5370, 5385 and 5371, 5385 and 5372, 5385 and 5373, 5385 and 5374, 5385 and 5375, 5385 and 5376, 5385 and 5377, 5385 and 5378, 5385 and 5379, 5385 and 5380, 5386 and 5370, 5386 and 5371, 5386 and 5372, 5386 and 5373, 5386 and 5374, 5386 and 5375, 5386 and 5376, 5386 and 5377, 5386 and 5378, 5386 and 5379, 5386 and 5380, 5387 and 5370, 5387 and 5371, 5387 and 5372, 5387 and 5373, 5387 and 5374, 5387 and 5375, 5387 and 5376, 5387 and 5377, 5387 and 5378, 5387 and 5379, 5387 and 5380, 5388 and 5370, 5388 and 5371, 5388 and 5372, 5388 and 5373, 5388 and 5374, 5388 and 5375, 5388 and 5376, 5388 and 5377, 5388 and 5378, 5388 and 5379, 5388 and 5380, 5389 and 5370, 5389 and 5371, 5389 and 5372, 5389 and 5373, 5389 and 5374, 5389 and 5375, 5389 and 5376, 5389 and 5377, 5389 and 5378, 5389 and 5379, 5389 and 5380, 5390 and 5370, 5390 and 5371, 5390 and 5372, 5390 and 5373, 5390 and 5374, 5390 and 5375, 5390 and 5376, 5390 and 5377, 5390 and 5378, 5390 and 5379, 5390 and 5380, 5391 and 5370, 5391 and 5371, 5391 and 5372, 5391 and 5373, 5391 and 5374, 5391 and 5375, 5391 and 5376, 5391 and 5377, 5391 and 5378, 5391 and 5379, 5391 and 5380, 5392 and 5370, 5392 and 5371, 5392 and 5372, 5392 and 5373, 5392 and 5374, 5392 and 5375, 5392 and 5376, 5392 and 5377, 5392 and 5378, 5392 and 5379, 5392 and 5380, 5393 and 5370, 5393 and 5371, 5393 and 5372, 5393 and 5373, 5393 and 5374, 5393 and 5375, 5393 and 5376, 5393 and 5377, 5393 and 5378, 5393 and 5379, 5393 and 5380, 5394 and 5370, 5394 and 5371, 5394 and 5372, 5394 and 5373, 5394 and 5374, 5394 and 5375, 5394 and 5376, 5394 and 5377, 5394 and 5378, 5394 and 5379, 5394 and 5380, 5395 and 5370, 5395 and 5371, 5395 and 5372, 5395 and 5373, 5395 and 5374, 5395 and 5375, 5395 and 5376, 5395 and 5377, 5395 and 5378, 5395 and 5379, 5395 and 5380, 5396 and 5370, 5396 and 5371, 5396 and 5372, 5396 and 5373, 5396 and 5374, 5396 and 5375, 5396 and 5376, 5396 and 5377, 5396 and 5378, 5396 and 5379, 5396 and 5380, 5397 and 5370, 5397 and 5371, 5397 and 5372, 5397 and 5373, 5397 and 5374, 5397 and 5375, 5397 and 5376, 5397 and 5377, 5397 and 5378, 5397 and 5379, 5397 and 5380, 5398 and 5370, 5398 and 5371, 5398 and 5372, 5398 and 5373, 5398 and 5374, 5398 and 5375, 5398 and 5376, 5398 and 5377, 5398 and 5378, 5398 and 5379, 5398 and 5380, 5399 and 5370, 5399 and 5371, 5399 and 5372, 5399 and 5373, 5399 and 5374, 5399 and 5375, 5399 and 5376, 5399 and 5377, 5399 and 5378, 5399 and 5379, 5399 and 5380, 5400 and 5370, 5400 and 5371, 5400 and 5372, 5400 and 5373, 5400 and 5374, 5400 and 5375, 5400 and 5376, 5400 and 5377, 5400 and 5378, 5400 and 5379, 5400 and 5380, 5401 and 5370, 5401 and 5371, 5401 and 5372, 5401 and 5373, 5401 and 5374, 5401 and 5375, 5401 and 5376, 5401 and 5377, 5401 and 5378, 5401 and 5379, or 5401 and 5380. The gRNA core of the PEgRNA can comprise SEQ ID NO. 5857-5859. Such PEgRNA sequences may further comprise a 3′ motif at the 3′ end of the extension arm, for example, a hairpin-forming motif or a series of 3, 4 or more U nucleotides. Without being bound by theory, such 3′ motifs are believed to increase PEgRNA stability. Such PEgRNA sequences may alternatively or additionally be adapted for transcription from a DNA template, for example, by including a 5′ terminal G if the spacer of the PEgRNA begins with another nucleotide, by including 6 or 7 U nucleotides at the 3′ end of the extension arm, or both. Such plasmid adapted sequences may further comprise a hairpin-forming motif between the PBS and the 3′ terminal U series.

Any of the PEgRNAs of Table 23 can be used in a Prime Editing system further comprising a nick guide RNA (ngRNA). Such ngRNA can comprise a spacer comprising nucleotides 5-20 of SEQ ID NO: 2048, 2052, 2070, 2080, 2085, 2086, 2088, 2089, 2091, 2423, 2424, 2425, 2426, 2427, 2428, 2429, 2430, 2431, 2432, 2433, 2434, 2435, 2436, 2438, 2439, 2440, 2443, 2444, 5402, 5403, 5404, or 5405 and a gRNA core capable of binding to a Cas9 protein. The spacer of the ngRNA can comprise nucleotides 4-20, 3-20, 2-20, or 1-20 of SEQ ID NO: 2048, 2052, 2070, 2080, 2085, 2086, 2088, 2089, 2091, 2423, 2424, 2425, 2426, 2427, 2428, 2429, 2430, 2431, 2432, 2433, 2434, 2435, 2436, 2438, 2439, 2440, 2443, 2444, 5402, 5403, 5404, or 5405. The spacer of the ngRNA can comprise SEQ ID NO: 2048, 2052, 2070, 2080, 2085, 2086, 2088, 2089, 2091, 2423, 2424, 2425, 2426, 2427, 2428, 2429, 2430, 2431, 2432, 2433, 2434, 2435, 2436, 2438, 2439, 2440, 2443, 2444, 5402, 5403, 5404, or 5405. The gRNA core of the ngRNA can comprise SEQ ID NO. 5857-5859. Exemplary ngRNA provided in Table 23 can comprise SEQ ID NO: 2257, 2259, 2260, 2261, 2262, 2264, 2270, 2272, 2274, 2275, 2276, 2278, 2280, 2282, 2283, 2284, 2285, 2288, 4411, 4412, 4413, 4415, 4416, 4417, 4419, 4420, or 4421. Such ngRNA sequences may further comprise a 3′ motif at the 3′ end of the gRNA core, for example, a series of 3, 4 or more U nucleotides. Exemplary ngRNA sequences with such 3′ adaptations include SEQ ID NOs: 2292 and 4424. Without being bound by theory, such 3′ motifs are believed to increase ngRNA stability.

Table 24 provides Prime Editing guide RNAs (PEgRNAs), which can be used in Prime Editing systems disclosed herein. Such Prime Editing systems can comprise a Cas9 protein capable of recognizing an NG PAM sequence (e.g., TG), and a reverse transcriptase. The Prime Editing systems (e.g., PE3 or PE3b systems) can further comprise a nick guide RNA (ngRNA). Such PEgRNAs and Prime Editing systems can be used, for example, to correct an H1069Q mutation in ATP7B.

The PEgRNAs of Table 24 comprise: (a) a spacer comprising nucleotides 5-20 of SEQ ID NO: 5406, (b) a gRNA core capable of binding to a Cas9 protein, and (c) an extension arm comprising: (i) an editing template comprising any one of SEQ ID NOs: 5418-5422, and (ii) a primer binding site (PBS) comprising any one of SEQ ID NOs: 5407-5417. The spacer of the PEgRNA can comprise, for example, nucleotides 4-20, 3-20, 2-20, or 1-20 of SEQ ID NO: 5406. The spacer of the PEgRNA can comprise SEQ ID NO: 5406. The RTT and the PBS can comprise respectively SEQ ID NOs: 5418 and 5407, 5418 and 5408, 5418 and 5409, 5418 and 5410, 5418 and 5411, 5418 and 5412, 5418 and 5413, 5418 and 5414, 5418 and 5415, 5418 and 5416, 5418 and 5417, 5419 and 5407, 5419 and 5408, 5419 and 5409, 5419 and 5410, 5419 and 5411, 5419 and 5412, 5419 and 5413, 5419 and 5414, 5419 and 5415, 5419 and 5416, 5419 and 5417, 5420 and 5407, 5420 and 5408, 5420 and 5409, 5420 and 5410, 5420 and 5411, 5420 and 5412, 5420 and 5413, 5420 and 5414, 5420 and 5415, 5420 and 5416, 5420 and 5417, 5421 and 5407, 5421 and 5408, 5421 and 5409, 5421 and 5410, 5421 and 5411, 5421 and 5412, 5421 and 5413, 5421 and 5414, 5421 and 5415, 5421 and 5416, 5421 and 5417, 5422 and 5407, 5422 and 5408, 5422 and 5409, 5422 and 5410, 5422 and 5411, 5422 and 5412, 5422 and 5413, 5422 and 5414, 5422 and 5415, 5422 and 5416, or 5422 and 5417. The gRNA core of the PEgRNA can comprise SEQ ID NO. 5857-5859. Such PEgRNA sequences may further comprise a 3′ motif at the 3′ end of the extension arm, for example, a hairpin-forming motif or a series of 3, 4 or more U nucleotides. Without being bound by theory, such 3′ motifs are believed to increase PEgRNA stability. Such PEgRNA sequences may alternatively or additionally be adapted for transcription from a DNA template, for example, by including a 5′ terminal G if the spacer of the PEgRNA begins with another nucleotide, by including 6 or 7 U nucleotides at the 3′ end of the extension arm, or both. Such plasmid adapted sequences may further comprise a hairpin-forming motif between the PBS and the 3′ terminal U series.

Any of the PEgRNAs of Table 24 can be used in a Prime Editing system further comprising a nick guide RNA (ngRNA). Such ngRNA can comprise a spacer comprising nucleotides 5-20 of SEQ ID NO: 18, 19, 20, 21, 22, 23, 24, 25, 26, 27, 28, 29, 30, 31, 32, 33, 34, 35, 36, 37, 38, 39, 40, 42, 43, 44, 45, 46, 47, 48, 49, 50, 51, 52, 53, 54, 55, 56, 57, 58, 59, 67, 68, 70, 72, 199, 200, 203, 529, 736, 737, 739, or 740 and a gRNA core capable of binding to a Cas9 protein. The spacer of the ngRNA can comprise nucleotides 4-20, 3-20, 2-20, or 1-20 of SEQ ID NO: 18, 19, 20, 21, 22, 23, 24, 25, 26, 27, 28, 29, 30, 31, 32, 33, 34, 35, 36, 37, 38, 39, 40, 42, 43, 44, 45, 46, 47, 48, 49, 50, 51, 52, 53, 54, 55, 56, 57, 58, 59, 67, 68, 70, 72, 199, 200, 203, 529, 736, 737, 739, or 740. The spacer of the ngRNA can comprise SEQ ID NO: 18, 19, 20, 21, 22, 23, 24, 25, 26, 27, 28, 29, 30, 31, 32, 33, 34, 35, 36, 37, 38, 39, 40, 42, 43, 44, 45, 46, 47, 48, 49, 50, 51, 52, 53, 54, 55, 56, 57, 58, 59, 67, 68, 70, 72, 199, 200, 203, 529, 736, 737, 739, or 740. The gRNA core of the ngRNA can comprise SEQ ID NO. 5857-5859. Exemplary ngRNA provided in Table 24 can comprise SEQ ID NO: 153, 154, 155, 156, 157, 158, 159, 160, 161, 162, 163, 164, 165, 166, 167, 168, 169, 170, 171, 172, 173, 174, 175, 176, 177, 178, 179, 180, 181, 290, 291, 292, 293, 681, 1501, 1503, or 1504. Such ngRNA sequences may further comprise a 3′ motif at the 3′ end of the gRNA core, for example, a series of 3, 4 or more U nucleotides. Without being bound by theory, such 3′ motifs are believed to increase ngRNA stability.

Table 25 provides Prime Editing guide RNAs (PEgRNAs), which can be used in Prime Editing systems disclosed herein. Such Prime Editing systems can comprise a Cas9 protein capable of recognizing an NG PAM sequence (e.g., GG), and a reverse transcriptase. The Prime Editing systems (e.g., PE3 or PE3b systems) can further comprise a nick guide RNA (ngRNA). Such PEgRNAs and Prime Editing systems can be used, for example, to correct an H1069Q mutation in ATP7B.

The PEgRNAs of Table 25 comprise: (a) a spacer comprising nucleotides 5-20 of SEQ ID NO: 5423, (b) a gRNA core capable of binding to a Cas9 protein, and (c) an extension arm comprising: (i) an editing template comprising any one of SEQ ID NOs: 5435-5445, and (ii) a primer binding site (PBS) comprising any one of SEQ ID NOs: 5424-5434. The spacer of the PEgRNA can comprise, for example, nucleotides 4-20, 3-20, 2-20, or 1-20 of SEQ ID NO: 5423. The spacer of the PEgRNA can comprise SEQ ID NO: 5423. The RTT and the PBS can comprise respectively SEQ ID NOs: 5435 and 5424, 5435 and 5425, 5435 and 5426, 5435 and 5427, 5435 and 5428, 5435 and 5429, 5435 and 5430, 5435 and 5431, 5435 and 5432, 5435 and 5433, 5435 and 5434, 5436 and 5424, 5436 and 5425, 5436 and 5426, 5436 and 5427, 5436 and 5428, 5436 and 5429, 5436 and 5430, 5436 and 5431, 5436 and 5432, 5436 and 5433, 5436 and 5434, 5437 and 5424, 5437 and 5425, 5437 and 5426, 5437 and 5427, 5437 and 5428, 5437 and 5429, 5437 and 5430, 5437 and 5431, 5437 and 5432, 5437 and 5433, 5437 and 5434, 5438 and 5424, 5438 and 5425, 5438 and 5426, 5438 and 5427, 5438 and 5428, 5438 and 5429, 5438 and 5430, 5438 and 5431, 5438 and 5432, 5438 and 5433, 5438 and 5434, 5439 and 5424, 5439 and 5425, 5439 and 5426, 5439 and 5427, 5439 and 5428, 5439 and 5429, 5439 and 5430, 5439 and 5431, 5439 and 5432, 5439 and 5433, 5439 and 5434, 5440 and 5424, 5440 and 5425, 5440 and 5426, 5440 and 5427, 5440 and 5428, 5440 and 5429, 5440 and 5430, 5440 and 5431, 5440 and 5432, 5440 and 5433, 5440 and 5434, 5441 and 5424, 5441 and 5425, 5441 and 5426, 5441 and 5427, 5441 and 5428, 5441 and 5429, 5441 and 5430, 5441 and 5431, 5441 and 5432, 5441 and 5433, 5441 and 5434, 5442 and 5424, 5442 and 5425, 5442 and 5426, 5442 and 5427, 5442 and 5428, 5442 and 5429, 5442 and 5430, 5442 and 5431, 5442 and 5432, 5442 and 5433, 5442 and 5434, 5443 and 5424, 5443 and 5425, 5443 and 5426, 5443 and 5427, 5443 and 5428, 5443 and 5429, 5443 and 5430, 5443 and 5431, 5443 and 5432, 5443 and 5433, 5443 and 5434, 5444 and 5424, 5444 and 5425, 5444 and 5426, 5444 and 5427, 5444 and 5428, 5444 and 5429, 5444 and 5430, 5444 and 5431, 5444 and 5432, 5444 and 5433, 5444 and 5434, 5445 and 5424, 5445 and 5425, 5445 and 5426, 5445 and 5427, 5445 and 5428, 5445 and 5429, 5445 and 5430, 5445 and 5431, 5445 and 5432, 5445 and 5433, or 5445 and 5434. The gRNA core of the PEgRNA can comprise SEQ ID NO. 5857-5859. Such PEgRNA sequences may further comprise a 3′ motif at the 3′ end of the extension arm, for example, a hairpin-forming motif or a series of 3, 4 or more U nucleotides. Without being bound by theory, such 3′ motifs are believed to increase PEgRNA stability. Such PEgRNA sequences may alternatively or additionally be adapted for transcription from a DNA template, for example, by including a 5′ terminal G if the spacer of the PEgRNA begins with another nucleotide, by including 6 or 7 U nucleotides at the 3′ end of the extension arm, or both. Such plasmid adapted sequences may further comprise a hairpin-forming motif between the PBS and the 3′ terminal U series.

Any of the PEgRNAs of Table 25 can be used in a Prime Editing system further comprising a nick guide RNA (ngRNA). Such ngRNA can comprise a spacer comprising nucleotides 5-20 of SEQ ID NO: 18, 19, 20, 21, 22, 23, 24, 25, 26, 27, 28, 29, 30, 31, 32, 33, 34, 35, 36, 37, 38, 39, 40, 42, 43, 44, 45, 46, 47, 48, 49, 50, 51, 52, 53, 54, 55, 56, 57, 58, 59, 67, 68, 70, 72, 199, 200, 203, 529, 736, 737, 738, 739, 740, or 5280 and a gRNA core capable of binding to a Cas9 protein. The spacer of the ngRNA can comprise nucleotides 4-20, 3-20, 2-20, or 1-20 of SEQ ID NO: 18, 19, 20, 21, 22, 23, 24, 25, 26, 27, 28, 29, 30, 31, 32, 33, 34, 35, 36, 37, 38, 39, 40, 42, 43, 44, 45, 46, 47, 48, 49, 50, 51, 52, 53, 54, 55, 56, 57, 58, 59, 67, 68, 70, 72, 199, 200, 203, 529, 736, 737, 738, 739, 740, or 5280. The spacer of the ngRNA can comprise SEQ ID NO: 18, 19, 20, 21, 22, 23, 24, 25, 26, 27, 28, 29, 30, 31, 32, 33, 34, 35, 36, 37, 38, 39, 40, 42, 43, 44, 45, 46, 47, 48, 49, 50, 51, 52, 53, 54, 55, 56, 57, 58, 59, 67, 68, 70, 72, 199, 200, 203, 529, 736, 737, 738, 739, 740, or 5280. The gRNA core of the ngRNA can comprise SEQ ID NO. 5857-5859. Exemplary ngRNA provided in Table 25 can comprise SEQ ID NO: 153, 154, 155, 156, 157, 158, 159, 160, 161, 162, 163, 164, 165, 166, 167, 168, 169, 170, 171, 172, 173, 174, 175, 176, 177, 178, 179, 180, 181, 290, 291, 292, 293, 681, 1501, 1502, 1503, or 1504. Such ngRNA sequences may further comprise a 3′ motif at the 3′ end of the gRNA core, for example, a series of 3, 4 or more U nucleotides. Without being bound by theory, such 3′ motifs are believed to increase ngRNA stability.

Table 26 provides Prime Editing guide RNAs (PEgRNAs), which can be used in Prime Editing systems disclosed herein. Such Prime Editing systems can comprise a Cas9 protein capable of recognizing an NG or NNNRRT PAM sequence (e.g., AG or AGCAGT), and a reverse transcriptase. The Prime Editing systems (e.g., PE3 or PE3b systems) can further comprise a nick guide RNA (ngRNA). Such PEgRNAs and Prime Editing systems can be used, for example, to correct an H1069Q mutation in ATP7B.

The PEgRNAs of Table 26 comprise: (a) a spacer comprising nucleotides 5-20 of SEQ ID NO: 5446, (b) a gRNA core capable of binding to a Cas9 protein, and (c) an extension arm comprising: (i) an editing template comprising any one of SEQ ID NOs: 5458-5472, and (ii) a primer binding site (PBS) comprising any one of SEQ ID NOs: 5447-5457. The spacer of the PEgRNA can comprise, for example, nucleotides 4-20, 3-20, 2-20, or 1-20 of SEQ ID NO: 5446. The spacer of the PEgRNA can comprise SEQ ID NO: 5446. The RTT and the PBS can comprise respectively SEQ ID NOs: 5458 and 5447, 5458 and 5448, 5458 and 5449, 5458 and 5450, 5458 and 5451, 5458 and 5452, 5458 and 5453, 5458 and 5454, 5458 and 5455, 5458 and 5456, 5458 and 5457, 5459 and 5447, 5459 and 5448, 5459 and 5449, 5459 and 5450, 5459 and 5451, 5459 and 5452, 5459 and 5453, 5459 and 5454, 5459 and 5455, 5459 and 5456, 5459 and 5457, 5460 and 5447, 5460 and 5448, 5460 and 5449, 5460 and 5450, 5460 and 5451, 5460 and 5452, 5460 and 5453, 5460 and 5454, 5460 and 5455, 5460 and 5456, 5460 and 5457, 5461 and 5447, 5461 and 5448, 5461 and 5449, 5461 and 5450, 5461 and 5451, 5461 and 5452, 5461 and 5453, 5461 and 5454, 5461 and 5455, 5461 and 5456, 5461 and 5457, 5462 and 5447, 5462 and 5448, 5462 and 5449, 5462 and 5450, 5462 and 5451, 5462 and 5452, 5462 and 5453, 5462 and 5454, 5462 and 5455, 5462 and 5456, 5462 and 5457, 5463 and 5447, 5463 and 5448, 5463 and 5449, 5463 and 5450, 5463 and 5451, 5463 and 5452, 5463 and 5453, 5463 and 5454, 5463 and 5455, 5463 and 5456, 5463 and 5457, 5464 and 5447, 5464 and 5448, 5464 and 5449, 5464 and 5450, 5464 and 5451, 5464 and 5452, 5464 and 5453, 5464 and 5454, 5464 and 5455, 5464 and 5456, 5464 and 5457, 5465 and 5447, 5465 and 5448, 5465 and 5449, 5465 and 5450, 5465 and 5451, 5465 and 5452, 5465 and 5453, 5465 and 5454, 5465 and 5455, 5465 and 5456, 5465 and 5457, 5466 and 5447, 5466 and 5448, 5466 and 5449, 5466 and 5450, 5466 and 5451, 5466 and 5452, 5466 and 5453, 5466 and 5454, 5466 and 5455, 5466 and 5456, 5466 and 5457, 5467 and 5447, 5467 and 5448, 5467 and 5449, 5467 and 5450, 5467 and 5451, 5467 and 5452, 5467 and 5453, 5467 and 5454, 5467 and 5455, 5467 and 5456, 5467 and 5457, 5468 and 5447, 5468 and 5448, 5468 and 5449, 5468 and 5450, 5468 and 5451, 5468 and 5452, 5468 and 5453, 5468 and 5454, 5468 and 5455, 5468 and 5456, 5468 and 5457, 5469 and 5447, 5469 and 5448, 5469 and 5449, 5469 and 5450, 5469 and 5451, 5469 and 5452, 5469 and 5453, 5469 and 5454, 5469 and 5455, 5469 and 5456, 5469 and 5457, 5470 and 5447, 5470 and 5448, 5470 and 5449, 5470 and 5450, 5470 and 5451, 5470 and 5452, 5470 and 5453, 5470 and 5454, 5470 and 5455, 5470 and 5456, 5470 and 5457, 5471 and 5447, 5471 and 5448, 5471 and 5449, 5471 and 5450, 5471 and 5451, 5471 and 5452, 5471 and 5453, 5471 and 5454, 5471 and 5455, 5471 and 5456, 5471 and 5457, 5472 and 5447, 5472 and 5448, 5472 and 5449, 5472 and 5450, 5472 and 5451, 5472 and 5452, 5472 and 5453, 5472 and 5454, 5472 and 5455, 5472 and 5456, or 5472 and 5457. The gRNA core of the PEgRNA can comprise SEQ ID NO, 5857-5859. Such PEgRNA sequences may further comprise a 3′ motif at the 3′ end of the extension arm, for example, a hairpin-forming motif or a series of 3, 4 or more U nucleotides. Without being bound by theory, such 3′ motifs are believed to increase PEgRNA stability. Such PEgRNA sequences may alternatively or additionally be adapted for transcription from a DNA template, for example, by including a 5′ terminal G if the spacer of the PEgRNA begins with another nucleotide, by including 6 or 7 U nucleotides at the 3′ end of the extension arm, or both. Such plasmid adapted sequences may further comprise a hairpin-forming motif between the PBS and the 3′ terminal U series.

Any of the PEgRNAs of Table 26 can be used in a Prime Editing system further comprising a nick guide RNA (ngRNA). Such ngRNA can comprise a spacer comprising nucleotides 5-20 of SEQ ID NO: 18, 19, 20, 21, 22, 23, 24, 25, 26, 27, 28, 29, 30, 31, 32, 33, 34, 35, 36, 37, 38, 39, 40, 42, 43, 44, 45, 46, 47, 48, 49, 50, 51, 52, 53, 54, 55, 56, 57, 58, 59, 67, 68, 70, 72, 199, 200, 201, 203, 529, 736, 737, 738, 739, 740, 5280, or 5281 and a gRNA core capable of binding to a Cas9 protein. The spacer of the ngRNA can comprise nucleotides 4-20, 3-20, 2-20, or 1-20 of SEQ ID NO: 18, 19, 20, 21, 22, 23, 24, 25, 26, 27, 28, 29, 30, 31, 32, 33, 34, 35, 36, 37, 38, 39, 40, 42, 43, 44, 45, 46, 47, 48, 49, 50, 51, 52, 53, 54, 55, 56, 57, 58, 59, 67, 68, 70, 72, 199, 200, 201, 203, 529, 736, 737, 738, 739, 740, 5280, or 5281. The spacer of the ngRNA can comprise SEQ ID NO: 18, 19, 20, 21, 22, 23, 24, 25, 26, 27, 28, 29, 30, 31, 32, 33, 34, 35, 36, 37, 38, 39, 40, 42, 43, 44, 45, 46, 47, 48, 49, 50, 51, 52, 53, 54, 55, 56, 57, 58, 59, 67, 68, 70, 72, 199, 200, 201, 203, 529, 736, 737, 738, 739, 740, 5280, or 5281. The gRNA core of the ngRNA can comprise SEQ ID NO. 5857-5859. Exemplary ngRNA provided in Table 26 can comprise SEQ ID NO: 153, 154, 155, 156, 157, 158, 159, 160, 161, 162, 163, 164, 165, 166, 167, 168, 169, 170, 171, 172, 173, 174, 175, 176, 177, 178, 179, 180, 181, 290, 291, 292, 293, 681, 1501, 1502, 1503, or 1504. Such ngRNA sequences may further comprise a 3′ motif at the 3′ end of the gRNA core, for example, a series of 3, 4 or more U nucleotides. Without being bound by theory, such 3′ motifs are believed to increase ngRNA stability.

Table 27 provides Prime Editing guide RNAs (PEgRNAs), which can be used in Prime Editing systems disclosed herein. Such Prime Editing systems can comprise a Cas9 protein capable of recognizing an NG PAM sequence (e.g., AG), and a reverse transcriptase. The Prime Editing systems (e.g., PE3 or PE3b systems) can further comprise a nick guide RNA (ngRNA). Such PEgRNAs and Prime Editing systems can be used, for example, to correct an H1069Q mutation in ATP7B.

The PEgRNAs of Table 27 comprise: (a) a spacer comprising nucleotides 5-20 of SEQ ID NO: 5473, (b) a gRNA core capable of binding to a Cas9 protein, and (c) an extension arm comprising: (i) an editing template comprising any one of SEQ ID NOs: 5485-5502, and (ii) a primer binding site (PBS) comprising any one of SEQ ID NOs: 5474-5484. The spacer of the PEgRNA can comprise, for example, nucleotides 4-20, 3-20, 2-20, or 1-20 of SEQ ID NO: 5473. The spacer of the PEgRNA can comprise SEQ ID NO: 5473. The RTT and the PBS can comprise respectively SEQ ID NOs: 5485 and 5474, 5485 and 5475, 5485 and 5476, 5485 and 5477, 5485 and 5478, 5485 and 5479, 5485 and 5480, 5485 and 5481, 5485 and 5482, 5485 and 5483, 5485 and 5484, 5486 and 5474, 5486 and 5475, 5486 and 5476, 5486 and 5477, 5486 and 5478, 5486 and 5479, 5486 and 5480, 5486 and 5481, 5486 and 5482, 5486 and 5483, 5486 and 5484, 5487 and 5474, 5487 and 5475, 5487 and 5476, 5487 and 5477, 5487 and 5478, 5487 and 5479, 5487 and 5480, 5487 and 5481, 5487 and 5482, 5487 and 5483, 5487 and 5484, 5488 and 5474, 5488 and 5475, 5488 and 5476, 5488 and 5477, 5488 and 5478, 5488 and 5479, 5488 and 5480, 5488 and 5481, 5488 and 5482, 5488 and 5483, 5488 and 5484, 5489 and 5474, 5489 and 5475, 5489 and 5476, 5489 and 5477, 5489 and 5478, 5489 and 5479, 5489 and 5480, 5489 and 5481, 5489 and 5482, 5489 and 5483, 5489 and 5484, 5490 and 5474, 5490 and 5475, 5490 and 5476, 5490 and 5477, 5490 and 5478, 5490 and 5479, 5490 and 5480, 5490 and 5481, 5490 and 5482, 5490 and 5483, 5490 and 5484, 5491 and 5474, 5491 and 5475, 5491 and 5476, 5491 and 5477, 5491 and 5478, 5491 and 5479, 5491 and 5480, 5491 and 5481, 5491 and 5482, 5491 and 5483, 5491 and 5484, 5492 and 5474, 5492 and 5475, 5492 and 5476, 5492 and 5477, 5492 and 5478, 5492 and 5479, 5492 and 5480, 5492 and 5481, 5492 and 5482, 5492 and 5483, 5492 and 5484, 5493 and 5474, 5493 and 5475, 5493 and 5476, 5493 and 5477, 5493 and 5478, 5493 and 5479, 5493 and 5480, 5493 and 5481, 5493 and 5482, 5493 and 5483, 5493 and 5484, 5494 and 5474, 5494 and 5475, 5494 and 5476, 5494 and 5477, 5494 and 5478, 5494 and 5479, 5494 and 5480, 5494 and 5481, 5494 and 5482, 5494 and 5483, 5494 and 5484, 5495 and 5474, 5495 and 5475, 5495 and 5476, 5495 and 5477, 5495 and 5478, 5495 and 5479, 5495 and 5480, 5495 and 5481, 5495 and 5482, 5495 and 5483, 5495 and 5484, 5496 and 5474, 5496 and 5475, 5496 and 5476, 5496 and 5477, 5496 and 5478, 5496 and 5479, 5496 and 5480, 5496 and 5481, 5496 and 5482, 5496 and 5483, 5496 and 5484, 5497 and 5474, 5497 and 5475, 5497 and 5476, 5497 and 5477, 5497 and 5478, 5497 and 5479, 5497 and 5480, 5497 and 5481, 5497 and 5482, 5497 and 5483, 5497 and 5484, 5498 and 5474, 5498 and 5475, 5498 and 5476, 5498 and 5477, 5498 and 5478, 5498 and 5479, 5498 and 5480, 5498 and 5481, 5498 and 5482, 5498 and 5483, 5498 and 5484, 5499 and 5474, 5499 and 5475, 5499 and 5476, 5499 and 5477, 5499 and 5478, 5499 and 5479, 5499 and 5480, 5499 and 5481, 5499 and 5482, 5499 and 5483, 5499 and 5484, 5500 and 5474, 5500 and 5475, 5500 and 5476, 5500 and 5477, 5500 and 5478, 5500 and 5479, 5500 and 5480, 5500 and 5481, 5500 and 5482, 5500 and 5483, 5500 and 5484, 5501 and 5474, 5501 and 5475, 5501 and 5476, 5501 and 5477, 5501 and 5478, 5501 and 5479, 5501 and 5480, 5501 and 5481, 5501 and 5482, 5501 and 5483, 5501 and 5484, 5502 and 5474, 5502 and 5475, 5502 and 5476, 5502 and 5477, 5502 and 5478, 5502 and 5479, 5502 and 5480, 5502 and 5481, 5502 and 5482, 5502 and 5483, or 5502 and 5484. The gRNA core of the PEgRNA can comprise SEQ ID NO. 5857-5859. Such PEgRNA sequences may further comprise a 3′ motif at the 3′ end of the extension arm, for example, a hairpin-forming motif or a series of 3, 4 or more U nucleotides. Without being bound by theory, such 3′ motifs are believed to increase PEgRNA stability. Such PEgRNA sequences may alternatively or additionally be adapted for transcription from a DNA template, for example, by including a 5′ terminal G if the spacer of the PEgRNA begins with another nucleotide, by including 6 or 7 U nucleotides at the 3′ end of the extension arm, or both. Such plasmid adapted sequences may further comprise a hairpin-forming motif between the PBS and the 3′ terminal U series.

Any of the PEgRNAs of Table 27 can be used in a Prime Editing system further comprising a nick guide RNA (ngRNA). Such ngRNA can comprise a spacer comprising nucleotides 5-20 of SEQ ID NO: 18, 19, 20, 21, 22, 23, 24, 25, 26, 27, 28, 29, 30, 31, 32, 33, 34, 35, 36, 37, 38, 39, 40, 42, 43, 44, 45, 46, 47, 48, 49, 50, 51, 52, 53, 54, 55, 56, 57, 58, 59, 67, 68, 70, 72, 199, 200, 203, 529, 736, 737, 738, 739, 740, 5280, or 5281 and a gRNA core capable of binding to a Cas9 protein. The spacer of the ngRNA can comprise nucleotides 4-20, 3-20, 2-20, or 1-20 of SEQ ID NO: 18, 19, 20, 21, 22, 23, 24, 25, 26, 27, 28, 29, 30, 31, 32, 33, 34, 35, 36, 37, 38, 39, 40, 42, 43, 44, 45, 46, 47, 48, 49, 50, 51, 52, 53, 54, 55, 56, 57, 58, 59, 67, 68, 70, 72, 199, 200, 203, 529, 736, 737, 738, 739, 740, 5280, or 5281. The spacer of the ngRNA can comprise SEQ ID NO: 18, 19, 20, 21, 22, 23, 24, 25, 26, 27, 28, 29, 30, 31, 32, 33, 34, 35, 36, 37, 38, 39, 40, 42, 43, 44, 45, 46, 47, 48, 49, 50, 51, 52, 53, 54, 55, 56, 57, 58, 59, 67, 68, 70, 72, 199, 200, 203, 529, 736, 737, 738, 739, 740, 5280, or 5281. The gRNA core of the ngRNA can comprise SEQ ID NO. 5857-5859. Exemplary ngRNA provided in Table 27 can comprise SEQ ID NO: 153, 154, 155, 156, 157, 158, 159, 160, 161, 162, 163, 164, 165, 166, 167, 168, 169, 170, 171, 172, 173, 174, 175, 176, 177, 178, 179, 180, 181, 290, 291, 292, 293, 681, 1501, 1502, 1503, or 1504. Such ngRNA sequences may further comprise a 3′ motif at the 3′ end of the gRNA core, for example, a series of 3, 4 or more U nucleotides. Without being bound by theory, such 3′ motifs are believed to increase ngRNA stability.

Table 28 provides Prime Editing guide RNAs (PEgRNAs), which can be used in Prime Editing systems disclosed herein. Such Prime Editing systems can comprise a Cas9 protein capable of recognizing an NG PAM sequence (e.g., TG), and a reverse transcriptase. The Prime Editing systems (e.g., PE3 or PE3b systems) can further comprise a nick guide RNA (ngRNA). Such PEgRNAs and Prime Editing systems can be used, for example, to correct an H1069Q mutation in ATP7B.

The PEgRNAs of Table 28 comprise: (a) a spacer comprising nucleotides 5-20 of SEQ ID NO: 5503, (b) a gRNA core capable of binding to a Cas9 protein, and (c) an extension arm comprising: (i) an editing template comprising any one of SEQ ID NOs: 5515-5535, and (ii) a primer binding site (PBS) comprising any one of SEQ ID NOs: 5504-5514. The spacer of the PEgRNA can comprise, for example, nucleotides 4-20, 3-20, 2-20, or 1-20 of SEQ ID NO: 5503. The spacer of the PEgRNA can comprise SEQ ID NO: 5503. The RTT and the PBS can comprise respectively SEQ ID NOs: 5515 and 5504, 5515 and 5505, 5515 and 5506, 5515 and 5507, 5515 and 5508, 5515 and 5509, 5515 and 5510, 5515 and 5511, 5515 and 5512, 5515 and 5513, 5515 and 5514, 5516 and 5504, 5516 and 5505, 5516 and 5506, 5516 and 5507, 5516 and 5508, 5516 and 5509, 5516 and 5510, 5516 and 5511, 5516 and 5512, 5516 and 5513, 5516 and 5514, 5517 and 5504, 5517 and 5505, 5517 and 5506, 5517 and 5507, 5517 and 5508, 5517 and 5509, 5517 and 5510, 5517 and 5511, 5517 and 5512, 5517 and 5513, 5517 and 5514, 5518 and 5504, 5518 and 5505, 5518 and 5506, 5518 and 5507, 5518 and 5508, 5518 and 5509, 5518 and 5510, 5518 and 5511, 5518 and 5512, 5518 and 5513, 5518 and 5514, 5519 and 5504, 5519 and 5505, 5519 and 5506, 5519 and 5507, 5519 and 5508, 5519 and 5509, 5519 and 5510, 5519 and 5511, 5519 and 5512, 5519 and 5513, 5519 and 5514, 5520 and 5504, 5520 and 5505, 5520 and 5506, 5520 and 5507, 5520 and 5508, 5520 and 5509, 5520 and 5510, 5520 and 5511, 5520 and 5512, 5520 and 5513, 5520 and 5514, 5521 and 5504, 5521 and 5505, 5521 and 5506, 5521 and 5507, 5521 and 5508, 5521 and 5509, 5521 and 5510, 5521 and 5511, 5521 and 5512, 5521 and 5513, 5521 and 5514, 5522 and 5504, 5522 and 5505, 5522 and 5506, 5522 and 5507, 5522 and 5508, 5522 and 5509, 5522 and 5510, 5522 and 5511, 5522 and 5512, 5522 and 5513, 5522 and 5514, 5523 and 5504, 5523 and 5505, 5523 and 5506, 5523 and 5507, 5523 and 5508, 5523 and 5509, 5523 and 5510, 5523 and 5511, 5523 and 5512, 5523 and 5513, 5523 and 5514, 5524 and 5504, 5524 and 5505, 5524 and 5506, 5524 and 5507, 5524 and 5508, 5524 and 5509, 5524 and 5510, 5524 and 5511, 5524 and 5512, 5524 and 5513, 5524 and 5514, 5525 and 5504, 5525 and 5505, 5525 and 5506, 5525 and 5507, 5525 and 5508, 5525 and 5509, 5525 and 5510, 5525 and 5511, 5525 and 5512, 5525 and 5513, 5525 and 5514, 5526 and 5504, 5526 and 5505, 5526 and 5506, 5526 and 5507, 5526 and 5508, 5526 and 5509, 5526 and 5510, 5526 and 5511, 5526 and 5512, 5526 and 5513, 5526 and 5514, 5527 and 5504, 5527 and 5505, 5527 and 5506, 5527 and 5507, 5527 and 5508, 5527 and 5509, 5527 and 5510, 5527 and 5511, 5527 and 5512, 5527 and 5513, 5527 and 5514, 5528 and 5504, 5528 and 5505, 5528 and 5506, 5528 and 5507, 5528 and 5508, 5528 and 5509, 5528 and 5510, 5528 and 5511, 5528 and 5512, 5528 and 5513, 5528 and 5514, 5529 and 5504, 5529 and 5505, 5529 and 5506, 5529 and 5507, 5529 and 5508, 5529 and 5509, 5529 and 5510, 5529 and 5511, 5529 and 5512, 5529 and 5513, 5529 and 5514, 5530 and 5504, 5530 and 5505, 5530 and 5506, 5530 and 5507, 5530 and 5508, 5530 and 5509, 5530 and 5510, 5530 and 5511, 5530 and 5512, 5530 and 55131, 5530 and 5514, 5531 and 5504, 5531 and 5505, 5531 and 5506, 5531 and 5507, 5531 and 5508, 5531 and 5509, 5531 and 5510, 5531 and 5511, 5531 and 5512, 5531 and 5513, 5531 and 5514, 5532 and 5504, 5532 and 5505, 5532 and 5506, 5532 and 5507, 5532 and 5508, 5532 and 5509, 5532 and 5510, 5532 and 5511, 5532 and 5512, 5532 and 5513, 5532 and 5514, 5533 and 5504, 5533 and 5505, 5533 and 5506, 5533 and 5507, 5533 and 5508, 5533 and 5509, 5533 and 5510, 5533 and 5511, 5533 and 5512, 5533 and 5513, 5533 and 5514, 5534 and 5504, 5534 and 5505, 5534 and 5506, 5534 and 5507, 5534 and 5508, 5534 and 5509, 5534 and 5510, 5534 and 5511, 5534 and 5512, 5534 and 5513, 5534 and 5514, 5535 and 5504, 5535 and 5505, 5535 and 5506, 5535 and 5507, 5535 and 5508, 5535 and 5509, 5535 and 5510, 5535 and 5511, 5535 and 5512, 5535 and 5513, or 5535 and 5514. The gRNA core of the PEgRNA can comprise SEQ ID NO. 5857-5859. Such PEgRNA sequences may further comprise a 3′ motif at the 3′ end of the extension arm, for example, a hairpin-forming motif or a series of 3, 4 or more U nucleotides. Without being bound by theory, such 3′ motifs are believed to increase PEgRNA stability. Such PEgRNA sequences may alternatively or additionally be adapted for transcription from a DNA template, for example, by including a 5′ terminal G if the spacer of the PEgRNA begins with another nucleotide, by including 6 or 7 U nucleotides at the 3′ end of the extension arm, or both. Such plasmid adapted sequences may further comprise a hairpin-forming motif between the PBS and the 3′ terminal U series.

Any of the PEgRNAs of Table 28 can be used in a Prime Editing system further comprising a nick guide RNA (ngRNA). Such ngRNA can comprise a spacer comprising nucleotides 5-20 of SEQ ID NO: 2045, 2046, 2047, 2048, 2049, 2050, 2051, 2052, 2053, 2054, 2055, 2056, 2057, 2058, 2059, 2060, 2061, 2062, 2063, 2064, 2065, 2066, 2067, 2068, 2069, 2070, 2071, 2072, 2073, 2074, 2075, 2076, 2077, 2078, 2079, 2080, 2081, 2082, 2083, 2084, 2085, 2086, 2087, 2088, 2089, 2090, 2091, 2092, 2093, 2094, 2095, 2096, 2437, 2439, 2442, 2443, 2444, or 5536 and a gRNA core capable of binding to a Cas9 protein. The spacer of the ngRNA can comprise nucleotides 4-20, 3-20, 2-20, or 1-20 of SEQ ID NO: 2045, 2046, 2047, 2048, 2049, 2050, 2051, 2052, 2053, 2054, 2055, 2056, 2057, 2058, 2059, 2060, 2061, 2062, 2063, 2064, 2065, 2066, 2067, 2068, 2069, 2070, 2071, 2072, 2073, 2074, 2075, 2076, 2077, 2078, 2079, 2080, 2081, 2082, 2083, 2084, 2085, 2086, 2087, 2088, 2089, 2090, 2091, 2092, 2093, 2094, 2095, 2096, 2437, 2439, 2442, 2443, 2444, or 5536. The spacer of the ngRNA can comprise SEQ ID NO: 2045, 2046, 2047, 2048, 2049, 2050, 2051, 2052, 2053, 2054, 2055, 2056, 2057, 2058, 2059, 2060, 2061, 2062, 2063, 2064, 2065, 2066, 2067, 2068, 2069, 2070, 2071, 2072, 2073, 2074, 2075, 2076, 2077, 2078, 2079, 2080, 2081, 2082, 2083, 2084, 2085, 2086, 2087, 2088, 2089, 2090, 2091, 2092, 2093, 2094, 2095, 2096, 2437, 2439, 2442, 2443, 2444, or 5536. The gRNA core of the ngRNA can comprise SEQ ID NO. 5857-5859. Exemplary ngRNA provided in Table 28 can comprise SEQ ID NO: 2257, 2258, 2259, 2260, 2261, 2262, 2263, 2264, 2265, 2266, 2267, 2268, 2269, 2270, 2271, 2272, 2273, 2274, 2275, 2276, 2277, 2278, 2279, 2280, 2281, 2282, 2283, 2284, 2285, 2286, 2287, 2288, 2289, 4411, 4416, 4418, 4420, or 4422. Such ngRNA sequences may further comprise a 3′ motif at the 3′ end of the gRNA core, for example, a series of 3, 4 or more U nucleotides. Exemplary ngRNA sequences with such 3′ adaptations include SEQ ID NOs: 2290-2292. Without being bound by theory, such 3′ motifs are believed to increase ngRNA stability.

Table 29 provides Prime Editing guide RNAs (PEgRNAs), which can be used in Prime Editing systems disclosed herein. Such Prime Editing systems can comprise a Cas9 protein capable of recognizing an NNGG PAM sequence (e.g., GCGG), and a reverse transcriptase. The Prime Editing systems (e.g., PE3 or PE3b systems) can further comprise a nick guide RNA (ngRNA). Such PEgRNAs and Prime Editing systems can be used, for example, to correct an H1069Q mutation in ATP7B.

The PEgRNAs of Table 29 comprise: (a) a spacer comprising nucleotides 5-20 of SEQ ID NO: 5537, (b) a gRNA core capable of binding to a Cas9 protein, and (c) an extension arm comprising: (i) an editing template comprising any one of SEQ ID NOs: 5549-5554, and (ii) a primer binding site (PBS) comprising any one of SEQ ID NOs: 5538-5548. The spacer of the PEgRNA can comprise, for example, nucleotides 4-20, 3-20, 2-20, or 1-20 of SEQ ID NO: 5537. The spacer of the PEgRNA can comprise SEQ ID NO: 5537. The RTT and the PBS can comprise respectively SEQ ID NOs: 5549 and 5538, 5549 and 5539, 5549 and 5540, 5549 and 5541, 5549 and 5542, 5549 and 5543, 5549 and 5544, 5549 and 5545, 5549 and 5546, 5549 and 5547, 5549 and 5548, 5550 and 5538, 5550 and 5539, 5550 and 5540, 5550 and 5541, 5550 and 5542, 5550 and 5543, 5550 and 5544, 5550 and 5545, 5550 and 5546, 5550 and 5547, 5550 and 5548, 5551 and 5538, 5551 and 5539, 5551 and 5540, 5551 and 5541, 5551 and 5542, 5551 and 5543, 5551 and 5544, 5551 and 5545, 5551 and 5546, 5551 and 5547, 5551 and 5548, 5552 and 5538, 5552 and 5539, 5552 and 5540, 5552 and 5541, 5552 and 5542, 5552 and 5543, 5552 and 5544, 5552 and 5545, 5552 and 5546, 5552 and 5547, 5552 and 5548, 5553 and 5538, 5553 and 5539, 5553 and 5540, 5553 and 5541, 5553 and 5542, 5553 and 5543, 5553 and 5544, 5553 and 5545, 5553 and 5546, 5553 and 5547, 5553 and 5548, 5554 and 5538, 5554 and 5539, 5554 and 5540, 5554 and 5541, 5554 and 5542, 5554 and 5543, 5554 and 5544, 5554 and 5545, 5554 and 5546, 5554 and 5547, or 5554 and 5548. The gRNA core of the PEgRNA can comprise SEQ ID NO. 5857-5859. Such PEgRNA sequences may further comprise a 3′ motif at the 3′ end of the extension arm, for example, a hairpin-forming motif or a series of 3, 4 or more U nucleotides. Without being bound by theory, such 3′ motifs are believed to increase PEgRNA stability. Such PEgRNA sequences may alternatively or additionally be adapted for transcription from a DNA template, for example, by including a 5′ terminal G if the spacer of the PEgRNA begins with another nucleotide, by including 6 or 7 U nucleotides at the 3′ end of the extension arm, or both. Such plasmid adapted sequences may further comprise a hairpin-forming motif between the PBS and the 3′ terminal U series.

Any of the PEgRNAs of Table 29 can be used in a Prime Editing system further comprising a nick guide RNA (ngRNA). Such ngRNA can comprise a spacer comprising nucleotides 5-20 of SEQ ID NO: 27, 30, 33, 35, 50, 68, 70, 200, 201, 202, 204, 205, 206, 207, 208, 209, 337, 736, 740, or 5306 and a gRNA core capable of binding to a Cas9 protein. The spacer of the ngRNA can comprise nucleotides 4-20, 3-20, 2-20, or 1-20 of SEQ ID NO: 27, 30, 33, 35, 50, 68, 70, 200, 201, 202, 204, 205, 206, 207, 208, 209, 337, 736, 740, or 5306. The spacer of the ngRNA can comprise SEQ ID NO: 27, 30, 33, 35, 50, 68, 70, 200, 201, 202, 204, 205, 206, 207, 208, 209, 337, 736, 740, or 5306. The gRNA core of the ngRNA can comprise SEQ ID NO. 5857-5859. Exemplary ngRNA provided in Table 29 can comprise SEQ ID NO: 153, 154, 157, 160, 162, 163, 165, 166, 167, 170, 171, 172, 173, 177, 178, 180, 181, 291, 292, 1501, 1503, or 1504. Such ngRNA sequences may further comprise a 3′ motif at the 3′ end of the gRNA core, for example, a series of 3, 4 or more U nucleotides. Without being bound by theory, such 3′ motifs are believed to increase ngRNA stability.

Table 30 provides Prime Editing guide RNAs (PEgRNAs), which can be used in Prime Editing systems disclosed herein. Such Prime Editing systems can comprise a Cas9 protein capable of recognizing an NGG or NG PAM sequence (e.g., TGG or TG), and a reverse transcriptase. The Prime Editing systems (e.g., PE3 or PE3b systems) can further comprise a nick guide RNA (ngRNA). Such PEgRNAs and Prime Editing systems can be used, for example, to correct an H1069Q mutation in ATP7B.

The PEgRNAs of Table 30 comprise: (a) a spacer comprising nucleotides 5-20 of SEQ ID NO: 5555, (b) a gRNA core capable of binding to a Cas9 protein, and (c) an extension arm comprising: (i) an editing template comprising any one of SEQ ID NOs: 5567-5590, and (ii) a primer binding site (PBS) comprising any one of SEQ ID NOs: 5556-5566. The spacer of the PEgRNA can comprise, for example, nucleotides 4-20, 3-20, 2-20, or 1-20 of SEQ ID NO: 5555. The spacer of the PEgRNA can comprise SEQ ID NO: 5555. The RTT and the PBS can comprise respectively SEQ ID NOs: 5567 and 5556, 5567 and 5557, 5567 and 5558, 5567 and 5559, 5567 and 5560, 5567 and 5561, 5567 and 5562, 5567 and 5563, 5567 and 5564, 5567 and 5565, 5567 and 5566, 5568 and 5556, 5568 and 5557, 5568 and 5558, 5568 and 5559, 5568 and 5560, 5568 and 5561, 5568 and 5562, 5568 and 5563, 5568 and 5564, 5568 and 5565, 5568 and 5566, 5569 and 5556, 5569 and 5557, 5569 and 5558, 5569 and 5559, 5569 and 5560, 5569 and 5561, 5569 and 5562, 5569 and 5563, 5569 and 5564, 5569 and 5565, 5569 and 5566, 5570 and 5556, 5570 and 5557, 5570 and 5558, 5570 and 5559, 5570 and 5560, 5570 and 5561, 5570 and 5562, 5570 and 5563, 5570 and 5564, 5570 and 5565, 5570 and 5566, 5571 and 5556, 5571 and 5557, 5571 and 5558, 5571 and 5559, 5571 and 5560, 5571 and 5561, 5571 and 5562, 5571 and 5563, 5571 and 5564, 5571 and 5565, 5571 and 5566, 5572 and 5556, 5572 and 5557, 5572 and 5558, 5572 and 5559, 5572 and 5560, 5572 and 5561, 5572 and 5562, 5572 and 5563, 5572 and 5564, 5572 and 5565, 5572 and 5566, 5573 and 5556, 5573 and 5557, 5573 and 5558, 5573 and 5559, 5573 and 5560, 5573 and 5561, 5573 and 5562, 5573 and 5563, 5573 and 5564, 5573 and 5565, 5573 and 5566, 5574 and 5556, 5574 and 5557, 5574 and 5558, 5574 and 5559, 5574 and 5560, 5574 and 5561, 5574 and 5562, 5574 and 5563, 5574 and 5564, 5574 and 5565, 5574 and 5566, 5575 and 5556, 5575 and 5557, 5575 and 5558, 5575 and 5559, 5575 and 5560, 5575 and 5561, 5575 and 5562, 5575 and 5563, 5575 and 5564, 5575 and 5565, 5575 and 5566, 5576 and 5556, 5576 and 5557, 5576 and 5558, 5576 and 5559, 5576 and 5560, 5576 and 5561, 5576 and 5562, 5576 and 5563, 5576 and 5564, 5576 and 5565, 5576 and 5566, 5577 and 5556, 5577 and 5557, 5577 and 5558, 5577 and 5559, 5577 and 5560, 5577 and 5561, 5577 and 5562, 5577 and 5563, 5577 and 5564, 5577 and 5565, 5577 and 5566, 5578 and 5556, 5578 and 5557, 5578 and 5558, 5578 and 5559, 5578 and 5560, 5578 and 5561, 5578 and 5562, 5578 and 5563, 5578 and 5564, 5578 and 5565, 5578 and 5566, 5579 and 5556, 5579 and 5557, 5579 and 5558, 5579 and 5559, 5579 and 5560, 5579 and 5561, 5579 and 5562, 5579 and 5563, 5579 and 5564, 5579 and 5565, 5579 and 5566, 5580 and 5556, 5580 and 5557, 5580 and 5558, 5580 and 5559, 5580 and 5560, 5580 and 5561, 5580 and 5562, 5580 and 5563, 5580 and 5564, 5580 and 5565, 5580 and 5566, 5581 and 5556, 5581 and 5557, 5581 and 5558, 5581 and 5559, 5581 and 5560, 5581 and 5561, 5581 and 5562, 5581 and 5563, 5581 and 5564, 5581 and 5565, 5581 and 5566, 5582 and 5556, 5582 and 5557, 5582 and 5558, 5582 and 5559, 5582 and 5560, 5582 and 5561, 5582 and 5562, 5582 and 5563, 5582 and 5564, 5582 and 5565, 5582 and 5566, 5583 and 5556, 5583 and 5557, 5583 and 5558, 5583 and 5559, 5583 and 5560, 5583 and 5561, 5583 and 5562, 5583 and 5563, 5583 and 5564, 5583 and 5565, 5583 and 5566, 5584 and 5556, 5584 and 5557, 5584 and 5558, 5584 and 5559, 5584 and 5560, 5584 and 5561, 5584 and 5562, 5584 and 5563, 5584 and 5564, 5584 and 5565, 5584 and 5566, 5585 and 5556, 5585 and 5557, 5585 and 5558, 5585 and 5559, 5585 and 5560, 5585 and 5561, 5585 and 5562, 5585 and 5563, 5585 and 5564, 5585 and 5565, 5585 and 5566, 5586 and 5556, 5586 and 5557, 5586 and 5558, 5586 and 5559, 5586 and 5560, 5586 and 5561, 5586 and 5562, 5586 and 5563, 5586 and 5564, 5586 and 5565, 5586 and 5566, 5587 and 5556, 5587 and 5557, 5587 and 5558, 5587 and 5559, 5587 and 5560, 5587 and 5561, 5587 and 5562, 5587 and 5563, 5587 and 5564, 5587 and 5565, 5587 and 5566, 5588 and 5556, 5588 and 5557, 5588 and 5558, 5588 and 5559, 5588 and 5560, 5588 and 5561, 5588 and 5562, 5588 and 5563, 5588 and 5564, 5588 and 5565, 5588 and 5566, 5589 and 5556, 5589 and 5557, 5589 and 5558, 5589 and 5559, 5589 and 5560, 5589 and 5561, 5589 and 5562, 5589 and 5563, 5589 and 5564, 5589 and 5565, 5589 and 5566, 5590 and 5556, 5590 and 5557, 5590 and 5558, 5590 and 5559, 5590 and 5560, 5590 and 5561, 5590 and 5562, 5590 and 5563, 5590 and 5564, 5590 and 5565, or 5590 and 5566. The gRNA core of the PEgRNA can comprise SEQ ID NO. 5857-5859. Exemplary PEgRNAs provided in Table 30 can comprise and one of SEQ ID NOs. 5591-5637. Such PEgRNA sequences may further comprise a 3 motif at the 3 end of the extension arm, for example, a hairpin-forming motif or a series of 3, 4 or more U nucleotides. Without being bound by theory, such 3′ motifs are believed to increase PEgRNA stability. Such PEgRNA sequences may alternatively or additionally be adapted for transcription from a DNA template, for example, by including a 5′ terminal G if the spacer of the PEgRNA begins with another nucleotide, by including 6 or 7 U nucleotides at the 3′ end of the extension arm, or both. Such plasmid adapted sequences may further comprise a hairpin-forming motif between the PBS and the 3′ terminal U series.

Any of the PEgRNAs of Table 30 can be used in a Prime Editing system further comprising a nick guide RNA (ngRNA). Such ngRNA can comprise a spacer comprising nucleotides 5-20 of SEQ ID NO: 41, 60, 61, 62, 63, 64, 65, 66, 69, or 71 and a gRNA core capable of binding to a Cas9 protein. The spacer of the ngRNA can comprise nucleotides 4-20, 3-20, 2-20, or 1-20 of SEQ ID NO: 41, 60, 61, 62, 63, 64, 65, 66, 69, or 71. The spacer of the ngRNA can comprise SEQ ID NO: 41, 60, 61, 62, 63, 64, 65, 66, 69, or 71. The gRNA core of the ngRNA can comprise SEQ ID NO. 5857-5859. Such ngRNA sequences may further comprise a 3′ motif at the 3′ end of the gRNA core, for example, a series of 3, 4 or more U nucleotides. Without being bound by theory, such 3′ motifs are believed to increase ngRNA stability.

Table 31 provides Prime Editing guide RNAs (PEgRNAs), which can be used in Prime Editing systems disclosed herein. Such Prime Editing systems can comprise a Cas9 protein capable of recognizing an NGG or NG PAM sequence (e.g., AGG or AG), and a reverse transcriptase. The Prime Editing systems (e.g., PE3 or PE3b systems) can further comprise a nick guide RNA (ngRNA). Such PEgRNAs and Prime Editing systems can be used, for example, to correct an H1069Q mutation in ATP7B.

The PEgRNAs of Table 31 comprise: (a) a spacer comprising nucleotides 5-20 of SEQ ID NO: 5638, (b) a gRNA core capable of binding to a Cas9 protein, and (c) an extension arm comprising: (i) an editing template comprising any one of SEQ ID NOs: 5650-5668, and (ii) a primer binding site (PBS) comprising any one of SEQ ID NOs: 5639-5649. The spacer of the PEgRNA can comprise, for example, nucleotides 4-20, 3-20, 2-20, or 1-20 of SEQ ID NO: 5638. The spacer of the PEgRNA can comprise SEQ ID NO: 5638. The RTT and the PBS can comprise respectively SEQ ID NOs: 5650 and 5639, 5650 and 5640, 5650 and 5641, 5650 and 5642, 5650 and 5643, 5650 and 5644, 5650 and 5645, 5650 and 5646, 5650 and 5647, 5650 and 5648, 5650 and 5649, 5651 and 5639, 5651 and 5640, 5651 and 5641, 5651 and 5642, 5651 and 5643, 5651 and 5644, 5651 and 5645, 5651 and 5646, 5651 and 5647, 5651 and 5648, 5651 and 5649, 5652 and 5639, 5652 and 5640, 5652 and 5641, 5652 and 5642, 5652 and 5643, 5652 and 5644, 5652 and 5645, 5652 and 5646, 5652 and 5647, 5652 and 5648, 5652 and 5649, 5653 and 5639, 5653 and 5640, 5653 and 5641, 5653 and 5642, 5653 and 5643, 5653 and 5644, 5653 and 5645, 5653 and 5646, 5653 and 5647, 5653 and 5648, 5653 and 5649, 5654 and 5639, 5654 and 5640, 5654 and 5641, 5654 and 5642, 5654 and 5643, 5654 and 5644, 5654 and 5645, 5654 and 5646, 5654 and 5647, 5654 and 5648, 5654 and 5649, 5655 and 5639, 5655 and 5640, 5655 and 5641, 5655 and 5642, 5655 and 5643, 5655 and 5644, 5655 and 5645, 5655 and 5646, 5655 and 5647, 5655 and 5648, 5655 and 5649, 5656 and 5639, 5656 and 5640, 5656 and 5641, 5656 and 5642, 5656 and 5643, 5656 and 5644, 5656 and 5645, 5656 and 5646, 5656 and 5647, 5656 and 5648, 5656 and 5649, 5657 and 5639, 5657 and 5640, 5657 and 5641, 5657 and 5642, 5657 and 5643, 5657 and 5644, 5657 and 5645, 5657 and 5646, 5657 and 5647, 5657 and 5648, 5657 and 5649, 5658 and 5639, 5658 and 5640, 5658 and 5641, 5658 and 5642, 5658 and 5643, 5658 and 5644, 5658 and 5645, 5658 and 5646, 5658 and 5647, 5658 and 5648, 5658 and 5649, 5659 and 5639, 5659 and 5640, 5659 and 5641, 5659 and 5642, 5659 and 5643, 5659 and 5644, 5659 and 5645, 5659 and 5646, 5659 and 5647, 5659 and 5648, 5659 and 5649, 5660 and 5639, 5660 and 5640, 5660 and 5641, 5660 and 5642, 5660 and 5643, 5660 and 5644, 5660 and 5645, 5660 and 5646, 5660 and 5647, 5660 and 5648, 5660 and 5649, 5661 and 5639, 5661 and 5640, 5661 and 5641, 5661 and 5642, 5661 and 5643, 5661 and 5644, 5661 and 5645, 5661 and 5646, 5661 and 5647, 5661 and 5648, 5661 and 5649, 5662 and 5639, 5662 and 5640, 5662 and 5641, 5662 and 5642, 5662 and 5643, 5662 and 5644, 5662 and 5645, 5662 and 5646, 5662 and 5647, 5662 and 5648, 5662 and 5649, 5663 and 5639, 5663 and 5640, 5663 and 5641, 5663 and 5642, 5663 and 5643, 5663 and 5644, 5663 and 5645, 5663 and 5646, 5663 and 5647, 5663 and 5648, 5663 and 5649, 5664 and 5639, 5664 and 5640, 5664 and 5641, 5664 and 5642, 5664 and 5643, 5664 and 5644, 5664 and 5645, 5664 and 5646, 5664 and 5647, 5664 and 5648, 5664 and 5649, 5665 and 5639, 5665 and 5640, 5665 and 5641, 5665 and 5642, 5665 and 5643, 5665 and 5644, 5665 and 5645, 5665 and 5646, 5665 and 5647, 5665 and 5648, 5665 and 5649, 5666 and 5639, 5666 and 5640, 5666 and 5641, 5666 and 5642, 5666 and 5643, 5666 and 5644, 5666 and 5645, 5666 and 5646, 5666 and 5647, 5666 and 5648, 5666 and 5649, 5667 and 5639, 5667 and 5640, 5667 and 5641, 5667 and 5642, 5667 and 5643, 5667 and 5644, 5667 and 5645, 5667 and 5646, 5667 and 5647, 5667 and 5648, 5667 and 5649, 5668 and 5639, 5668 and 5640, 5668 and 5641, 5668 and 5642, 5668 and 5643, 5668 and 5644, 5668 and 5645, 5668 and 5646, 5668 and 5647, 5668 and 5648, or 5668 and 5649. The gRNA core of the PEgRNA can comprise SEQ ID NO. 5857-5859. Exemplary PEgRNAs provided in Table 31 can comprise any one of SEQ ID NOs. 5669-5705. Such PEgRNA sequences may further comprise a 3′ motif at the 3′ end of the extension arm, for example, a hairpin-forming motif or a series of 3, 4 or more U nucleotides. Without being bound by theory, such 3′ motifs are believed to increase PEgRNA stability. Such PEgRNA sequences may alternatively or additionally be adapted for transcription from a DNA template, for example, by including a 5′ terminal G if the spacer of the PEgRNA begins with another nucleotide, by including 6 or 7 U nucleotides at the 3′ end of the extension arm, or both. Exemplary transcription-adapted sequences include None. Such plasmid adapted sequences may further comprise a hairpin-forming motif between the PBS and the 3′ terminal U series.

Any of the PEgRNAs of Table 31 can be used in a Prime Editing system further comprising a nick guide RNA (ngRNA). Such ngRNA can comprise a spacer comprising nucleotides 5-20 of SEQ ID NO: 41, 60, 61, 62, 63, 64, 65, 66, 69, or 71 and a gRNA core capable of binding to a Cas9 protein. The spacer of the ngRNA can comprise nucleotides 4-20, 3-20, 2-20, or 1-20 of SEQ ID NO: 41, 60, 61, 62, 63, 64, 65, 66, 69, or 71. The spacer of the ngRNA can comprise SEQ ID NO: 41, 60, 61, 62, 63, 64, 65, 66, 69, or 71. The gRNA core of the ngRNA can comprise SEQ ID NO. 5857-5859. Such ngRNA sequences may further comprise a 3′ motif at the 3′ end of the gRNA core, for example, a series of 3, 4 or more U nucleotides. Without being bound by theory, such 3′ motifs are believed to increase ngRNA stability.

Table 32 provides Prime Editing guide RNAs (PEgRNAs), which can be used in Prime Editing systems disclosed herein. Such Prime Editing systems can comprise a Cas9 protein capable of recognizing an NGG or NG PAM sequence (e.g., AGG or AG), and a reverse transcriptase. The Prime Editing systems (e.g., PE3 or PE3b systems) can further comprise a nick guide RNA (ngRNA). Such PEgRNAs and Prime Editing systems can be used, for example, to correct an H1069Q mutation in ATP7B.

The PEgRNAs of Table 32 comprise: (a) a spacer comprising nucleotides 5-20 of SEQ ID NO: 5706. (b) a gRNA core capable of binding to a Cas9 protein, and (c) an extension arm comprising: (i) an editing template comprising any one of SEQ ID NOs: 5718-5738, and (ii) a primer binding site (PBS) comprising any one of SEQ ID NOs: 5707-5717. The spacer of the PEgRNA can comprise, for example, nucleotides 4-20, 3-20, 2-20, or 1-20 of SEQ ID NO: 5706. The spacer of the PEgRNA can comprise SEQ ID NO: 5706. The RTT and the PBS can comprise respectively SEQ ID NOs: 5718 and 5707, 5718 and 5708, 5718 and 5709, 5718 and 5710, 5718 and 5711, 5718 and 5712, 5718 and 5713, 5718 and 5714, 5718 and 5715, 5718 and 5716, 5718 and 5717, 5719 and 5707, 5719 and 5708, 5719 and 5709, 5719 and 5710, 5719 and 5711, 5719 and 5712, 5719 and 5713, 5719 and 5714, 5719 and 5715, 5719 and 5716, 5719 and 5717, 5720 and 5707, 5720 and 5708, 5720 and 5709, 5720 and 5710, 5720 and 5711, 5720 and 5712, 5720 and 5713, 5720 and 5714, 5720 and 5715, 5720 and 5716, 5720 and 5717, 5721 and 5707, 5721 and 5708, 5721 and 5709, 5721 and 5710, 5721 and 5711, 5721 and 5712, 5721 and 5713, 5721 and 5714, 5721 and 5715, 5721 and 5716, 5721 and 5717, 5722 and 5707, 5722 and 5708, 5722 and 5709, 5722 and 5710, 5722 and 5711, 5722 and 5712, 5722 and 5713, 5722 and 5714, 5722 and 5715, 5722 and 5716, 5722 and 5717, 5723 and 5707, 5723 and 5708, 5723 and 5709, 5723 and 5710, 5723 and 5711, 5723 and 5712, 5723 and 5713, 5723 and 5714, 5723 and 5715, 5723 and 5716, 5723 and 5717, 5724 and 5707, 5724 and 5708, 5724 and 5709, 5724 and 5710, 5724 and 5711, 5724 and 5712, 5724 and 5713, 5724 and 5714, 5724 and 5715, 5724 and 5716, 5724 and 5717, 5725 and 5707, 5725 and 5708, 5725 and 5709, 5725 and 5710, 5725 and 5711, 5725 and 5712, 5725 and 5713, 5725 and 5714, 5725 and 5715, 5725 and 5716, 5725 and 5717, 5726 and 5707, 5726 and 5708, 5726 and 5709, 5726 and 5710, 5726 and 5711, 5726 and 5712, 5726 and 5713, 5726 and 5714, 5726 and 57151, 5726 and 5716, 5726 and 5717, 5727 and 5707, 5727 and 5708, 5727 and 5709, 5727 and 5710, 5727 and 5711, 5727 and 5712, 5727 and 5713, 5727 and 5714, 5727 and 5715, 5727 and 5716, 5727 and 5717, 5728 and 5707, 5728 and 5708, 5728 and 5709, 5728 and 5710, 5728 and 5711, 5728 and 5712, 5728 and 5713, 5728 and 5714, 5728 and 5715, 5728 and 5716, 5728 and 5717, 5729 and 5707, 5729 and 5708, 5729 and 5709, 5729 and 5710, 5729 and 5711, 5729 and 5712, 5729 and 5713, 5729 and 57141, 5729 and 5715, 5729 and 5716, 5729 and 5717, 5730 and 5707, 5730 and 5708, 5730 and 5709, 5730 and 5710, 5730 and 5711, 5730 and 5712, 5730 and 5713, 5730 and 5714, 5730 and 5715, 5730 and 5716, 5730 and 5717, 5731 and 5707, 5731 and 5708, 5731 and 5709, 5731 and 5710, 5731 and 5711, 5731 and 5712, 5731 and 5713, 5731 and 5714, 5731 and 5715, 5731 and 5716, 5731 and 5717, 5732 and 5707, 5732 and 5708, 5732 and 5709, 5732 and 5710, 5732 and 5711, 5732 and 5712, 5732 and 5713, 5732 and 5714, 5732 and 5715, 5732 and 5716, 5732 and 5717, 5733 and 5707, 5733 and 5708, 5733 and 5709, 5733 and 5710, 5733 and 5711, 5733 and 5712, 5733 and 5713, 5733 and 5714, 5733 and 5715, 5733 and 5716, 5733 and 5717, 5734 and 5707, 5734 and 5708, 5734 and 5709, 5734 and 5710, 5734 and 5711, 5734 and 5712, 5734 and 5713, 5734 and 5714, 5734 and 5715, 5734 and 5716, 5734 and 5717, 5735 and 5707, 5735 and 5708, 5735 and 5709, 5735 and 5710, 5735 and 5711, 5735 and 5712, 5735 and 5713, 5735 and 5714, 5735 and 5715, 5735 and 5716, 5735 and 5717, 5736 and 5707, 5736 and 5708, 5736 and 5709, 5736 and 5710, 5736 and 5711, 5736 and 5712, 5736 and 5713, 5736 and 5714, 5736 and 5715, 5736 and 5716, 5736 and 5717, 5737 and 5707, 5737 and 5708, 5737 and 5709, 5737 and 5710, 5737 and 5711, 5737 and 5712, 5737 and 5713, 5737 and 5714, 5737 and 5715, 5737 and 5716, 5737 and 5717, 5738 and 5707, 5738 and 5708, 5738 and 5709, 5738 and 5710, 5738 and 5711, 5738 and 5712, 5738 and 5713, 5738 and 5714, 5738 and 5715, 5738 and 5716, or 5738 and 5717. The gRNA core of the PEgRNA can comprise SEQ ID NO. 5857-5859. Exemplary PEgRNAs provided in Table 32 can comprise any one of SEQ ID NOs. 5739-5779. Such PEgRNA sequences may further comprise a 3′ motif at the 3′ end of the extension arm, for example, a hairpin-forming motif or a series of 3, 4 or more U nucleotides. Without being bound by theory, such 3′ motifs are believed to increase PEgRNA stability. Such PEgRNA sequences may alternatively or additionally be adapted for transcription from a DNA template, for example, by including a 5′ terminal G if the spacer of the PEgRNA begins with another nucleotide, by including 6 or 7 U nucleotides at the 3′ end of the extension arm, or both. Such plasmid adapted sequences may further comprise a hairpin-forming motif between the PBS and the 3′ terminal U series.

Any of the PEgRNAs of Table 32 can be used in a Prime Editing system further comprising a nick guide RNA (ngRNA). Such ngRNA can comprise a spacer comprising nucleotides 5-20 of SEQ ID NO: 41, 60, 61, 62, 63, 64, 65, 66, 69, or 71 and a gRNA core capable of binding to a Cas9 protein. The spacer of the ngRNA can comprise nucleotides 4-20, 3-20, 2-20, or 1-20 of SEQ ID NO: 41, 60, 61, 62, 63, 64, 65, 66, 69, or 71. The spacer of the ngRNA can comprise SEQ ID NO: 41, 60, 61, 62, 63, 64, 65, 66, 69, or 71. The gRNA core of the ngRNA can comprise SEQ ID NO. 5857-5859. Such ngRNA sequences may further comprise a 3′ motif at the 3′ end of the gRNA core, for example, a series of 3, 4 or more U nucleotides. Without being bound by theory, such 3′ motifs are believed to increase ngRNA stability.

Table 33 provides Prime Editing guide RNAs (PEgRNAs) and nick guide RNAs (ngRNAs) that can be used in Prime Editing systems disclosed herein. Any of the PEgRNAs of Table 33 can be used in a Prime Editing system further comprising any ngRNA of Table 33.

TABLE 33
exemplary PEgRNA and ngRNA sequences.

| SEQ | | |
|---|---|---|
| ID | DESCRIP- | |
| NO. | TION | SEQUENCE |
| 2739 | PEgRNA | UUUGGUGACUGCCACGCCCAGUUUUAGAGCUAGAAA |
| | | UAGCAAGUUAAAAUAAGGCUAGUCCGUUAUCAACUU |
| | | GAAAAAGUGGCACCGAGUCGGUGCAGUGAACAcCCCU |
| | | UGGGCGUGGCAuuuu |
| 3277 | PEgRNA | UUUGGUGACUGCCACGCCCAGUUUUAGAGCUAGAAA |
| | | UAGCAAGUUAAAAUAAGGCUAGUCCGUUAUCAACUU |
| | | GAAAAAGUGGCACCGAGUCGGUGCAGUGAACAcCCgU |
| | | UGGGCGUGGCAGUCACCAAuuuu |
| 3276 | PEgRNA | UUUGGUGACUGCCACGCCCAGUUUUAGAGCUAGAAA |
| | | UAGCAAGUUAAAAUAAGGCUAGUCCGUUAUCAACUU |
| | | GAAAAAGUGGCACCGAGUCGGUGCAGUGAACAcCCCU |
| | | UGGGCGUGGCAGUCACCAAuuuu |
| 2785 | PERNA | UUUGGUGACUGCCACGCCCAGUUUUAGAGCUAGAAA |
| | | UAGCAAGUUAAAAUAAGGCUAGUCCGUUAUCAACUU |
| | | GAAAAAGUGGCACCGAGUCGGUGCGAACAcCCCUUGG |
| | | GCGUGGCAGUCAuuuu |
| 4695 | PEgRNA | UUGGUGACUGCCACGCCCAAGUUUUAGAGCUAGAAA |
| | | UAGCAAGUUAAAAUAAGGCUAGUCCGUUAUCAACUU |
| | | GAAAAAGUGGCACCGAGUCGGUGCGUGAACAcCCCUU |
| | | GGGCGUGGCAGUCuuuu |
| 4762 | PERNA | UUGGUGACUGCCACGCCCAAGUUUUAGAGCUAGAAA |
| | | UAGCAAGUUAAAAUAAGGCUAGUCCGUUAUCAACUU |
| | | GAAAAAGUGGCACCGAGUCGGUGCAGUGAACAcCCCU |
| | | UGGGCGUGGCAGUCAuuuu |
| 4824 | PEgRNA | UUGGUGACUGCCACGCCCAAGUUUUAGAGCUAGAAA |
| | | UAGCAAGUUAAAAUAAGGCUAGUCCGUUAUCAACUU |
| | | GAAAAAGUGGCACCGAGUCGGUGCGCAGUGAACAcCC |
| | | CUUGGGCGUGGCAGUCAuuuu |
| 4721 | PEgRNA | UUGGUGACUGCCACGCCCAAGUUUUAGAGCUAGAAA |
| | | UAGCAAGUUAAAAUAAGGCUAGUCCGUUAUCAACUU |
| | | GAAAAAGUGGCACCGAGUCGGUGCAGUGAACAcCCCU |
| | | UGGGCGUGGCAGUuuuu |
| 4743 | PEgRNA | UUGGUGACUGCCACGCCCAAGUUUUAGAGCUAGAAA |
| | | UAGCAAGUUAAAAUAAGGCUAGUCCGUUAUCAACUU |
| | | GAAAAAGUGGCACCGAGUCGGUGCAGUGAACAcCCCU |
| | | UGGGCGUGGCAGUCuuuu |
| 4536 | PEgRNA | UUGGUGACUGCCACGCCCAAGUUUUAGAGCUAGAAA |
| | | UAGCAAGUUAAAAUAAGGCUAGUCCGUUAUCAACUU |
| | | GAAAAAGUGGCACCGAGUCGGUGCAACAcCCCUUGGG |
| | | CGUGGCuuuu |
| 4741 | PEgRNA | UUGGUGACUGCCACGCCCAAGUUUUAGAGCUAGAAA |
| | | UAGCAAGUUAAAAUAAGGCUAGUCCGUUAUCAACUU |
| | | GAAAAAGUGGCACCGAGUCGGUGCUGAACAcCCCUUG |
| | | GGCGUGGCAGUCACuuuu |
| 4788 | PEgRNA | UUGGUGACUGCCACGCCCAAGUUUUAGAGCUAGAAA |
| | | UAGCAAGUUAAAAUAAGGCUAGUCCGUUAUCAACUU |
| | | GAAAAAGUGGCACCGAGUCGGUGCAGCAGUGAACAcC |
| | | CCUUGGGCGUGGCAGuuuu |
| 4613 | PEgRNA | UUGGUGACUGCCACGCCCAAGUUUUAGAGCUAGAAA |
| | | UAGCAAGUUAAAAUAAGGCUAGUCCGUUAUCAACUU |
| | | GAAAAAGUGGCACCGAGUCGGUGCUGAACAcCCCUUG |
| | | GGCGUGGCAGuuuu |
| 2291 | ngRNA | GCCAGCAGUGAACAcCCCUUGUUUUAGAGCUAGAAAU |
| | | AGCAAGUUAAAAUAAGGCUAGUCCGUUAUCAACUUG |
| | | AAAAAGUGGCACCGAGUCGGUGCuuuu |
| 2292 | ngRNA | GGCCAGCAGUGAACAcCCCUGUUUUAGAGCUAGAAAU |
| | | AGCAAGUUAAAAUAAGGCUAGUCCGUUAUCAACUUG |
| | | AAAAAGUGGCACCGAGUCGGUGCuuuu |
| 4423 | ngRNA | GCCAGCAGUGAACAcCCGUUGUUUUAGAGCUAGAAA |
| | | UAGCAAGUUAAAAUAAGGCUAGUCCGUUAUCAACUU |
| | | GAAAAAGUGGCACCGAGUCGGUGCuuuu |
| 4424 | ngRNA | GGCCAGCAGUGAACAcCCGUGUUUUAGAGCUAGAAA |
| | | UAGCAAGUUAAAAUAAGGCUAGUCCGUUAUCAACUU |
| | | GAAAAAGUGGCACCGAGUCGGUGCuuuu |
| 2290 | ngRNA | CAUGCGGGUGCUCCUGCUGGGUUUUAGAGCUAGAAA |
| | | UAGCAAGUUAAAAUAAGGCUAGUCCGUUAUCAACUU |
| | | GAAAAAGUGGCACCGAGUCGGUGCuuuu |

A PEgRNA and/or an ngRNA of this disclosure, in some embodiments, may include modified nucleotides, e.g., chemically modified DNA or RNA nucleobases, and may include one or more nucleobase analogs (e.g., modifications which might add functionality, such as temperature resilience). In some embodiments, PEgRNAs and/or ngRNAs as described herein may be chemically modified. The phrase “chemical modifications,” as used herein, can include modifications which introduce chemistries which differ from those seen in naturally occurring DNA or RNAs, for example, covalent modifications such as the introduction of modified nucleotides, (e.g., nucleotide analogs, or the inclusion of pendant groups which are not naturally found in DNA or RNA molecules).

In some embodiments, the PEgRNAs and/or ngRNAs provided in this disclosure may have undergone a chemical or biological modifications. Modifications may be made at any position within a PEgRNA or ngRNA, and may include modification to a nucleobase or to a phosphate backbone of the PEgRNA or ngRNA. In some embodiments, chemical modifications can be a structure guided modifications. In some embodiments, a chemical modification is at the 5′ end and/or the 3′ end of a PEgRNA. In some embodiments, a chemical modification is at the 5′ end and/or the 3′ end of a ngRNA. In some embodiments, a chemical modification may be within the spacer sequence, the extension arm, the editing template sequence, or the primer binding site of a PEgRNA. In some embodiments, a chemical modification may be within the spacer sequence or the gRNA core of a PEgRNA or a ngRNA. In some embodiments, a chemical modification may be within the 3′ most nucleotides of a PEgRNA or ngRNA. In some embodiments, a chemical modification may be within the 3′ most end of a PEgRNA or ngRNA. In some embodiments, a chemical modification may be within the 5′ most end of a PEgRNA or ngRNA. In some embodiments, a PEgRNA or ngRNA comprises 1, 2, 3, 4, 5, 6, 7, 8, 9, 10 or more chemically modified nucleotides at the 3′ end. In some embodiments, a PEgRNA or ngRNA comprises 1, 2, 3, 4, 5, 6, 7, 8, 9, 10 or more chemically modified nucleotides at the 5′ end. In some embodiments, a PEgRNA or ngRNA comprises 1, 2, 3, 4, or 5 or more chemically modified nucleotides at the 3′ end. In some embodiments, a PEgRNA or ngRNA comprises 1, 2, 3, 4, or 5 more chemically modified nucleotides at the 5′ end. In some embodiments, a PEgRNA or ngRNA comprises 1, 2, or 3 or more chemically modified nucleotides at the 3′ end. In some embodiments, a PEgRNA or ngRNA comprises 1, 2, or 3 more chemically modified nucleotides at the 5′ end. In some embodiments, a PEgRNA or ngRNA comprises 1, 2, 3, 4, 5, 6, 7, 8, 9, 10 or more contiguous chemically modified nucleotides at the 3′ end. In some embodiments, a PEgRNA or ngRNA comprises 1, 2, 3, 4, 5, 6, 7, 8, 9, 10 or more contiguous chemically modified nucleotides at the 5′ end. In some embodiments, a PEgRNA or ngRNA comprises 1, 2, 3, 4, or 5 contiguous chemically modified nucleotides at the 3′ end. In some embodiments, a PEgRNA or ngRNA comprises 1, 2, 3, 4, or 5 contiguous chemically modified nucleotides at the 5′ end. In some embodiments, a PEgRNA or ngRNA comprises 1, 2, or 3 contiguous chemically modified nucleotides at the 3′ end. In some embodiments, a PEgRNA or ngRNA comprises 1, 2, or 3 contiguous chemically modified nucleotides at the 5′ end. In some embodiments, a PEgRNA or ngRNA comprises 3 contiguous chemically modified nucleotides at the 3′ end. In some embodiments, a PEgRNA or ngRNA comprises 1, 2, 3, 4, 5, or more chemically modified nucleotides near the 3′ end. In some embodiments, a PEgRNA or ngRNA comprises 3 contiguous chemically modified nucleotides at the 3′ end. In some embodiments, a PEgRNA or ngRNA comprises 3 contiguous chemically modified nucleotides at the 5′ end. In some embodiments, a PEgRNA or ngRNA comprises 1, 2, 3, 4, 5, or more chemically modified nucleotides near the 3′ end. In some embodiments, a PEgRNA or ngRNA comprises 1, 2, 3, 4, 5, or more contiguous chemically modified nucleotides near the 3′ end. In some embodiments, a PEgRNA or ngRNA comprises 1, 2, 3, 4, 5, or more chemically modified nucleotides near the 3′ end, where the 3′ most nucleotide is not modified, and the 1, 2, 3, 4, 5, or more chemically modified nucleotides precede the 3′ most nucleotide in a 5′-to-3′ order. In some embodiments, a PEgRNA or ngRNA comprises 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35 or more chemically modified nucleotides near the 3′ end, where the 3′ most nucleotide is not modified, and the 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35 or more chemically modified nucleotides precede the 3′ most nucleotide in a 5′-to-3′ order.

In some embodiments, a PEgRNA or ngRNA comprises one or more chemical modified nucleotides in the gRNA core. As exemplified in FIG. 5, the gRNA core of a PEgRNA may comprise one or more regions of a base paired lower stein, a base paired upper stem, where the lower stein and tipper stem may be connected by a bulge comprising unpaired RNAs. The gRNA core may further comprise a nexus distal from the spacer sequence. In some embodiments, the gRNA core comprises one or more chemically modified nucleotides in the lower stem, upper stein, and/or the hairpin regions. In some embodiments, all of the nucleotides in the lower stem, upper stem, and/or the hairpin regions are chemically modified.

A chemical modification to a PEgRNA or ngRNA can comprise a 2′-O-thionocarbamate-protected nucleoside phosphoramidite, a 2′-O-methyl (M), a 2′-O-methyl 3′ phosphorothioate (MS), or a 2′-O-methyl 3′ thioPACE (MSP), or any combination thereof. In some embodiments, a chemically modified PEgRNA and/or ngRNA can comprise a ′—O-methyl (M) RNA, a 2′-O-methyl 3′ phosphorothioate (MS) RNA, a 2′-O-methyl 3′ thioPACE (MSP) RNA, a 2′-F RNA, a phosphorothioate bond modification, any other chemical modifications known in the art, or any combination thereof. A chemical modification may also include, for example, the incorporation of non-nucleotide linkages or modified nucleotides into the PEgRNA and/or ngRNA (e.g., modifications to one or both of the 3′ and 5′ ends of a guide RNA molecule). Such modifications can include the addition of bases to an RNA sequence, complexing the RNA with an agent (e.g., a protein or a complementary nucleic acid molecule), and inclusion of elements which change the structure of an RNA molecule (e.g., which form secondary structures).

### Prime Editing Compositions

Disclosed herein, in some embodiments, are compositions, systems, and methods using a prime editing composition. The term “prime editing composition” or “prime editing system” refers to compositions involved in the method of prime editing as described herein. A prime editing composition may include a prime editor, e.g., a prime editor fusion protein, and a PEgRNA. A prime editing composition may further comprise additional elements, such as second strand nicking ngRNAs. Components of a prime editing composition may be combined to form a complex for prime editing, or may be kept separately, e.g., for administration purposes. In some embodiments, a prime editing composition comprises a prime editor fusion protein complexed with a PEgRNA and optionally complexed with a ngRNA. In some embodiments, the prime editing composition comprises a prime editor comprising a DNA binding domain and a DNA polymerase domain associated with each other through a PEgRNA. For example, the prime editing composition may comprise a prime editor comprising a DNA binding domain and a DNA polymerase domain linked to each other by an RNA-protein recruitment aptamer RNA sequence, which is linked to a PEgRNA. In some embodiments, a prime editing composition comprises a PEgRNA and a polynucleotide, a polynucleotide construct, or a vector that encodes a prime editor fusion protein. In some embodiments, a prime editing composition comprises a PEgRNA, a ngRNA, and a polynucleotide, a polynucleotide construct, or a vector that encodes a prime editor fusion protein. In some embodiments, a prime editing composition comprises multiple polynucleotides, polynucleotide constructs, or vectors, each of which encodes one or more prime editing composition components. In some embodiments, the PEgRNA of a prime editing composition is associated with the DNA binding domain, e.g., a Cas9 nickase, of the prime editor. In some embodiments, the PEgRNA of a prime editing composition complexes with the DNA binding domain of a prime editor and directs the prime editor to the target DNA.

In some embodiments, a prime editing composition comprises one or more polynucleotides that encode prime editor components and/or PEgRNA or ngRNAs. In some embodiments, a prime editing composition comprises a polynucleotide encoding a fusion protein comprising a DNA binding domain and a DNA polymerase domain. In some embodiments, a prime editing composition comprises (i) a polynucleotide encoding a fusion protein comprising a DNA binding domain and a DNA polymerase domain, and (ii) a PEgRNA or a polynucleotide encoding the PEgRNA. In some embodiments, a prime editing composition comprises (i) a polynucleotide encoding a fusion protein comprising a DNA binding domain and a DNA polymerase domain, (ii) a PEgRNA or a polynucleotide encoding the PEgRNA, and (iii) an ngRNA or a polynucleotide encoding the ngRNA. In some embodiments, a prime editing composition comprises (i) a polynucleotide encoding a DNA binding domain of a prime editor, e.g., a Cas9 nickase, (ii) a polynucleotide encoding a DNA polymerase domain of a prime editor, e.g., a reverse transcriptase, and (iii) a PEgRNA or a polynucleotide encoding the PEgRNA. In some embodiments, a prime editing composition comprises (i) a polynucleotide encoding a DNA binding domain of a prime editor, e.g., a Cas9 nickase, (ii) a polynucleotide encoding a DNA polymerase domain of a prime editor, e.g., a reverse transcriptase, (iii) a PEgRNA or a polynucleotide encoding the PEgRNA, and (iv) an ngRNA or a polynucleotide encoding the ngRNA. In some embodiments, the polynucleotide encoding the DNA biding domain or the polynucleotide encoding the DNA polymerase domain further encodes an additional polypeptide domain, e.g., an RNA-protein recruitment domain, such as a MS2 coat protein domain. In some embodiments, a prime editing composition comprises (i) a polynucleotide encoding a N-terminal half of a prime editor fusion protein and an intein-N and (ii) a polynucleotide encoding a C-terminal half of a prime editor fusion protein and an intein-C. In some embodiments, a prime editing composition comprises (i) a polynucleotide encoding a N-terminal half of a prime editor fusion protein and an intein-N (ii) a polynucleotide encoding a C-terminal half of a prime editor fusion protein and an intein-C, (iii) a PEgRNA or a polynucleotide encoding the PEgRNA, and/or (iv) an ngRNA or a polynucleotide encoding the ngRNA. In some embodiments, a prime editing composition comprises (i) a polynucleotide encoding a N-terminal portion of a DNA binding domain and an intein-N, (ii) a polynucleotide encoding a C-terminal portion of the DNA binding domain, an intein-C, and a DNA polymerase domain. In some embodiments, the DNA binding domain is a Cas protein domain, e.g., a Cas9 nickase. In some embodiments, the prime editing composition comprises (i) a polynucleotide encoding a N-terminal portion of a DNA binding domain and an intein-N, (ii) a polynucleotide encoding a C-terminal portion of the DNA binding domain, an intein-C, and a DNA polymerase domain, (iii) a PEgRNA or a polynucleotide encoding the PEgRNA, and/or (iv) a ngRNA or a polynucleotide encoding the ngRNA.

In some embodiments, a prime editing system comprises one or more polynucleotides encoding one or more prime editor polypeptides, wherein activity of the prime editing system can be temporally regulated by controlling the timing in which the vectors are delivered. For example, in some embodiments, a polynucleotide encoding the prime editor and a polynucleotide encoding a PEgRNA can be delivered simultaneously. For example, in some embodiments, a polynucleotide encoding the prime editor and a polynucleotide encoding a PEgRNA can be delivered sequentially.

In some embodiments, a polynucleotide encoding a component of a prime editing system can further comprise an element that is capable of modifying the intracellular half-life of the polynucleotide and/or modulating translational control. In some embodiments, the polynucleotide is a RNA, for example, an mRNA. In some embodiments, the half-life of the polynucleotide, e.g., the RNA may be increased. In some embodiments, the half-life of the polynucleotide, e.g., the RNA may be decreased. In some embodiments, the element may be capable of increasing the stability of the polynucleotide, e.g., the RNA. In some embodiments, the element may be capable of decreasing the stability of the polynucleotide, e.g., the RNA. In some embodiments, the element may be within the 3′ UTR of the RNA. In some embodiments, the element may include a polyadenylation signal (PA). In some embodiments, the element may include a cap, e.g., an upstream mRNA or PEgRNA end. In some embodiments, the RNA may comprise no PA such that it is subject to quicker degradation in the cell after transcription.

In some embodiments, the element may include at least one AU-rich element (ARE). The AREs may be bound by ARE binding proteins (ARE-BPs) in a manner that is dependent upon tissue type, cell type, timing, cellular localization, and environment. In some embodiments the destabilizing element may promote RNA decay, affect RNA stability, or activate translation. In some embodiments, the ARE may comprise 50 to 150 nucleotides in length. In some embodiments, the ARE may comprise at least one copy of the sequence AUUUA. In some embodiments, at least one ARE may be added to the 3′ UTR of the RNA. In some embodiments, the element may be a Woodchuck Hepatitis Virus Posttranscriptional Regulatory Element (WPRE). In further embodiments, the element is a modified and/or truncated WPRE sequence that is capable of enhancing expression from the transcript. In some embodiments, the WPRE or equivalent may be added to the 3′ UTR of the RNA. In some embodiments, the element may be selected from other RNA sequence motifs that are enriched in either fast- or slow-decaying transcripts. In some embodiments, the polynucleotide, e.g., a vector, encoding the PE or the PEgRNA may be self-destroyed via cleavage of a target sequence present on the polynucleotide, e.g., a vector. The cleavage may prevent continued transcription of a PE or a PEgRNA.

Polynucleotides encoding prime editing composition components can be DNA, RNA, or any combination thereof. In some embodiments, a polynucleotide encoding a prime editing composition component is an expression construct. In some embodiments, a polynucleotide encoding a prime editing composition component is a vector. In some embodiments, the vector is a DNA vector. In some embodiments, the vector is a plasmid. In some embodiments, the vector is a virus vector, e.g., a retroviral vector, adenoviral vector, lentiviral vector, herpesvirus vector, or an adeno-associated virus vector (AAV).

In some embodiments, polynucleotides encoding polypeptide components of a prime editing composition are codon optimized by replacing at least one codon (e.g., about or more than about 1, 2, 3, 4, 5, 10, 15, 20, 25, 50, or more codons) of the native sequence with codons that are more frequently or most frequently used in the genes of that host cell while maintaining the native amino acid sequence. In some embodiments, a polynucleotide encoding a polypeptide component of a prime editing composition are operably linked to one or more expression regulatory elements, for example, a promoter, a 3′ UTR, a 5′ UTR, or any combination thereof. In some embodiments, a polynucleotide encoding a prime editing composition component is a messenger RNA (mRNA). In some embodiments, the mRNA comprises a Cap at the 5′ end and/or a poly A tail at the 3′ end.

In some embodiments, the PEgRNA as described herein comprises a spacer that comprises a sequence selected from the group consisting of SEQ ID Nos. 1, 182, 294, 483, 682, 1505, 2023, 2293, 4425, 5206, 5228, 5248, 5282, 5313, 5340, 5369, 5406, 5423, 5446, 5473, 5503, 5537, 5555, 5638, and 5706.

In some embodiments, the PEgRNA as described herein comprises a editing template that comprises a sequence selected from the group consisting of SEQ ID Nos.: 13-17, 194-198, 306-336, 495-528, 694-735, 1517-1546, 2035-2044, 2305-2422, 4437-4492, 5218, 5240-5247, 5260-5279, 5294-5302, 5325-5338, 5352-5368, 5381-5401, 5418-5422, 5435-5445, 5458-5472, 5485-5502, 5515-5535, 5549-5554, 5567-5590, 5650-5668, and 5718-5738.

In some embodiments, the PEgRNA as described herein comprises a PBS that comprises a sequence selected from the group consisting of SEQ ID Nos. 2-12, 183-193, 295-305, 484-494, 683-693, 1506-1516, 2024-2034, 2294-2304, 4426-4436, 5207-5217, 5229-5239, 5249-5259, 5283-5293, 5314-5324, 5341-5351, 5370-5380, 5407-5417, 5424-5434, 5447-5457, 5474-5484, 5504-5514, 5538-5548, 5556-5566, 5639-5649, and 5707-5717.

In some embodiments, the PEgRNA as described herein comprises a sequence selected from the group consisting of SEQ ID Nos. 73-152, 210-289, 338-482, 530-680, 741-1500, 1547-2022, 2097-2256, 2445-4409, 4493-5205, 5591-5637, 5669-5705, and 5739-5779.

In some embodiments, the ngRNA disclosed herein comprises a ng spacer that comprises a sequence selected from the group consisting of SEQ ID Nos. 18-72, 199-209, 337, 529, 736-740, 2045-2096, 2423-2444, 5219-5227, 5280-5281, 5303-5312, 5339, 5402-5405, and 5536.

In some embodiments, the ngRNA disclosed herein comprises a sequence selected from the group consisting of SEQ ID NOs: 153-181.

### Pharmaceutical Compositions

Disclosed herein are pharmaceutical compositions comprising any of the prime editing composition components, for example, prime editors, fusion proteins, polynucleotides encoding prime editor polypeptides, PEgRNAs, ngRNAs, and/or prime editing complexes described herein.

The term “pharmaceutical composition”, as used herein, refers to a composition formulated for pharmaceutical use. In some embodiments, the pharmaceutical composition further comprises a pharmaceutically acceptable carrier. In some embodiments, the pharmaceutical composition comprises additional agents, e.g., for specific delivery, increasing half-life, or other therapeutic compounds.

In some embodiments, a pharmaceutically-acceptable carrier comprises any vehicle, such as a liquid or solid filler, diluent, excipient, manufacturing aid (e.g., lubricant, tale magnesium, calcium or zinc stearate, or steric acid), or solvent encapsulating material, involved in carrying or transporting the compound from one site (e.g., the delivery site) of the body, to another site (e.g., organ, tissue or portion of the body). A pharmaceutically acceptable carrier is “acceptable” in the sense of being compatible with the other ingredients of the formulation and not injurious to the tissue of the subject (e.g., physiologically compatible, sterile, physiologic pH, etc.).

Formulations of the pharmaceutical compositions described herein can be prepared by any method known or hereafter developed in the art of pharmacology. In general, such preparatory methods include the step of bringing the active ingredient(s) into association with an excipient and/or one or more other accessory ingredients, and then, if necessary and/or desirable, shaping and/or packaging the product into a desired single- or multi-dose unit. Pharmaceutical formulations can additionally comprise a pharmaceutically acceptable excipient, which, as used herein, includes any and all solvents, dispersion media, diluents, or other liquid vehicles, dispersion or suspension aids, surface active agents, isotonic agents, thickening or emulsifying agents, preservatives, solid binders, lubricants and the like, as suited to the particular dosage form desired.

### Methods of Editing

The methods and compositions disclosed herein can be used to edit a target gene of interest by prime editing.

In some embodiments, the prime editing method comprises contacting a target gene, e.g., an ATP7B gene, with a PEgRNA and a prime editor (PE) polypeptide described herein. In some embodiments, the target gene is double stranded, and comprises two strands of DNA complementary to each other. In some embodiments, the contacting with a PEgRNA and the contacting with a prime editor are performed sequentially. In some embodiments, the contacting with a prime editor is performed after the contacting with a PEgRNA. In some embodiments, the contacting with a PEgRNA is performed after the contacting with a prime editor. In some embodiments, the contacting with a PEgRNA, and the contacting with a prime editor are performed simultaneously. In some embodiments, the PEgRNA and the prime editor are associated in a complex prior to contacting a target gene.

In some embodiments, contacting the target gene with the prime editing composition results in binding of the PEgRNA to a target strand of the target gene, e.g., an ATP7B gene. In some embodiments, contacting the target gene with the prime editing composition results in binding of the PEgRNA to a search target sequence on the target strand of the target gene upon contacting with the PEgRNA. In some embodiments, contacting the target gene with the prime editing composition results in binding of a spacer sequence of the PEgRNA to a search target sequence with the search target sequence on the target strand of the target gene upon said contacting of the PEgRNA.

In some embodiments, contacting the target gene with the prime editing composition results in binding of the prime editor to the target gene, e.g. the target ATP7B gene, upon the contacting of the PE composition with the target gene. In some embodiments, the DNA binding domain of the PE associates with the PEgRNA. In some embodiments, the PE binds the target gene, e.g. an ATP7B gene, directed by the PEgRNA. Accordingly, in some embodiments, the contacting of the target gene result in binding of a DNA binding domain of a prime editor of the target ATP7B gene directed by the PEgRNA.

In some embodiments, contacting the target gene with the prime editing composition results in a nick in an edit strand of the target gene, e.g. an ATP7B gene by the prime editor upon contacting with the target gene, thereby generating a nicked on the edit strand of the target gene. In some embodiments, contacting the target gene with the prime editing composition results in a single-stranded DNA comprising a free 3′ end at the nick site of the edit strand of the target gene. In some embodiments, contacting the target gene with the prime editing composition results in a nick in the edit strand of the target gene by a DNA binding domain of the prime editor, thereby generating a single-stranded DNA comprising a free 3′ end at the nick site. In some embodiments, the DNA binding domain of the prime editor is a Cas domain. In some embodiments, the DNA binding domain of the prime editor is a Cas9. In some embodiments, the DNA binding domain of the prime editor is a Cas9 nickase.

In some embodiments, contacting the target gene with the prime editing composition results in hybridization of the PEgRNA with the 3′ end of the nicked single-stranded DNA, thereby priming DNA polymerization by a DNA polymerase domain of the prime editor. In some embodiments, the free 3′ end of the single-stranded DNA generated at the nick site hybridizes to a primer binding site sequence (PBS) of the contacted PEgRNA, thereby priming DNA polymerization. In some embodiments, the DNA polymerization is reverse transcription catalyzed by a reverse transcriptase domain of the prime editor. In some embodiments, the method comprises contacting the target gene with a DNA polymerase, e.g., a reverse transcriptase, as a pail of a prime editor fusion protein or prime editing complex (in cis), or as a separate protein (in trans).

In some embodiments, contacting the target gene with the prime editing composition generates an edited single stranded DNA that is coded by the editing template of the PEgRNA by DNA polymerase mediated polymerization from the 3′ free end of the single-stranded DNA at the nick site. In some embodiments, the editing template of the PEgRNA comprises one or more intended nucleotide edits compared to endogenous sequence of the target gene, e.g., an ATP7B gene. In some embodiments, the intended nucleotide edits are incorporated in the target gene, by excision of the 5′ single stranded DNA of the edit strand of the target gene generated at the nick site and DNA repair. In some embodiments, the intended nucleotide edits are incorporated in the target gene by excision of the editing target sequence and DNA repair. In some embodiments, excision of the 5′ single stranded DNA of the edit strand generated at the nick site is by a flap endonuclease. In some embodiments, the flap nuclease is FEN1. In some embodiments, the method further comprises contacting the target gene with a flap endonuclease. In some embodiments, the flap endonuclease is provided as a part of a prime editor fusion protein. In some embodiments, the flap endonuclease is provided in trans.

In some embodiments, contacting the target gene with the prime editing composition generates a mismatched heteroduplex comprising the edit strand of the target gene that comprises the edited single stranded DNA, and the unedited target strand of the target gene. Without being bound by theory, the endogenous DNA repair and replication may resolve the mismatched edited DNA to incorporate the nucleotide change(s) to form the desired edited target gene.

In some embodiments, the method further comprises contacting the target gene, e.g. an ATP7B gene, with a nick guide (ngRNA) disclosed herein. In some embodiments, the ngRNA comprises a spacer that binds a second search target sequence on the edit strand of the target gene. In some embodiments, the contacted ngRNA directs the PE to introduce a nick in the target strand of the target gene. In some embodiments, the nick on the target strand (non-edit strand) results in endogenous DNA repair machinery to use the edit strand to repair the non-edit strand, thereby incorporating the intended nucleotide edit in both strand of the target gene and modifying the target gene. In some embodiments, the ngRNA comprises a spacer sequence that is complementary to, and may hybridize with, the second search target sequence on the edit strand only after the intended nucleotide edit(s) are incorporated in the edit strand of the target gene.

In some embodiments, the target gene is contacted by the ngRNA, the PEgRNA, and the IE simultaneously. In some embodiments, the ngRNA, the PEgRNA, and the PE form a complex when they contact the target gene. In some embodiments, the target gene is contacted with the ngRNA, the PEgRNA, and the prime editor sequentially. In some embodiments, the target gene is contacted with the ngRNA and/or the PEgRNA after contacting the target gene with the PE. In some embodiments, the target gene is contacted with the ngRNA and/or the PEgRNA before contacting the target gene with the prime editor.

In some embodiments, the target gene. e.g., an ATP7B gene, is in a cell. Accordingly, also provided herein are methods of modifying a cell.

In some embodiments, the prime editing method comprises introducing a PEgRNA, a prime editor, and/or a ngRNA into the cell that has the target gene. In some embodiments, the prime editing method comprises introducing into the cell that has the target gene with a prime editing composition comprising a PEgRNA, a prime editor polypeptide, and/or a ngRNA. In some embodiments, the PEgRNA, the prime editor polypeptide, and/or the ngRNA form a complex prior to the introduction into the cell. In some embodiments, the PEgRNA, the prime editor polypeptide, and/or the ngRNA form a complex after the introduction into the cell. The prime editors, PEgRNA and/or ngRNAs, and prime editing complexes may be introduced into the cell by any delivery approaches described herein or any delivery approach known in the art, including ribonucleoprotein (RNPs), lipid nanoparticles (LNPs), viral vectors, non-viral vectors, mRNA delivery, and physical techniques such as cell membrane disruption by a microfluidics device. The prime editors, PEgRNA and/or ngRNAs. and prime editing complexes may be introduced into the cell simultaneously or sequentially.

In some embodiments, the prime editing method comprises introducing into the cell a PEgRNA or a polynucleotide encoding the PEgRNA, a prime editor polynucleotide encoding a prime editor polypeptide, and optionally an ngRNA or a polynucleotide encoding the ngRNA. In some embodiments, the method comprises introducing the PEgRNA or the polynucleotide encoding the PEgRNA, the polynucleotide encoding the prime editor polypeptide, and/or the ngRNA or the polynucleotide encoding the ngRNA into the cell simultaneously. In some embodiments, the method comprises introducing the PEgRNA or the polynucleotide encoding the PEgRNA, the polynucleotide encoding the prime editor polypeptide, and/or the ngRNA or the polynucleotide encoding the ngRNA into the cell sequentially. In some embodiments, the method comprises introducing the polynucleotide encoding the prime editor polypeptide into the cell before introduction of the PEgRNA or the polynucleotide encoding the PEgRNA and/or the ngRNA or the polynucleotide encoding the ngRNA. In some embodiments, the polynucleotide encoding the prime editor polypeptide is introduced into and expressed in the cell before introduction of the PEgRNA or the polynucleotide encoding the PEgRNA and/or the ngRNA or the polynucleotide encoding the ngRNA into the cell. In some embodiments, the polynucleotide encoding the prime editor polypeptide is introduced into the cell after the PEgRNA or the polynucleotide encoding the PEgRNA and/or the ngRNA or the polynucleotide encoding the ngRNA are introduced into the cell. The polynucleotide encoding the prime editor polypeptide, the PEgRNA or the polynucleotide encoding the PEgRNA, and/or the ngRNA or the polynucleotide encoding the ngRNA, may be introduced into the cell by any delivery approaches described herein or any delivery approach known in the art, for example, by RNPs, LNPs, viral vectors, non-viral vectors, mRNA delivery, and physical.

In some embodiments, the polynucleotide encoding the prime editor polypeptide, the poly nucleotide encoding the PEgRNA, and/or the polynucleotide encoding the ngRNA integrate into the genome of the cell after being introduced into the cell. In some embodiments, the polynucleotide encoding the prime editor polypeptide, the polynucleotide encoding the PEgRNA, and/or the polynucleotide encoding the ngRNA are introduced into the cell for transient expression. Accordingly, also provided herein are cells modified by prime editing.

In some embodiments, the cell is a prokaryotic cell. In some embodiments, the cell is a eukaryotic cell. In some embodiments, the cell is a mammalian cell. In some embodiments, the cell is a non-human primate cell, bovine cell, porcine cell, rodent or mouse cell. In some embodiments, the cell is a human cell. In some embodiments, the cell is a primary cell. In some embodiments, the cell is a human primary cell. In some embodiments, the cell is a progenitor cell. In some embodiments, the cell is a human progenitor cell. In some embodiments, the cell is a hepatocyte. In some embodiments, the cell is a human hepatocyte. In some embodiments, the cell is a primary human hepatocyte derived from an induced human pluripotent stem cell (iPSC). In some embodiments, the cell is a neuron. In some embodiments, the cell is a neuron from basal ganglia. In some embodiments, the cell is a neuron from basal ganglia of a subject.

In some embodiments, the target gene edited by prime editing is in a chromosome of the cell. In some embodiments, the intended nucleotide edits incorporate in the chromosome of the cell and are inheritable by progeny cells. In some embodiments, the intended nucleotide edits introduced to the cell by the prime editing compositions and methods are such that the cell and progeny of the cell also include the intended nucleotide edits. In some embodiments, the cell is autologous, allogeneic, or xenogeneic to a subject. In some embodiments, the cell is from or derived from a subject. In some embodiments, the cell is from or derived from a human subject. In some embodiments, the cell is introduced back into the subject, e.g., a human subject, after incorporation of the intended nucleotide edits by prime editing.

In some embodiments, the method provided herein comprises introducing the prime editor polypeptide or the polynucleotide encoding the prime editor polypeptide, the PEgRNA or the polynucleotide encoding the PEgRNA, and/or the ngRNA or the polynucleotide encoding the ngRNA into a plurality or a population of cells that comprise the target gene. In some embodiments, the population of cells is of the same cell type. In some embodiments, the population of cells is of the same tissue or organ. In some embodiments, the population of cells is heterogeneous. In some embodiments, the population of cells is homogeneous. In some embodiments, the population of cells is from a single tissue or organ, and the cells are heterogeneous. In some embodiments, the introduction into the population of cells is ex vivo. In some embodiments, the introduction into the population of cells is in vivo, e.g., into a human subject.

In some embodiments, the target gene is in a genome of each cell of the population. In some embodiments, introduction of the prime editor polypeptide or the polynucleotide encoding the prime editor polypeptide, the PEgRNA or the polynucleotide encoding the PEgRNA, and/or the ngRNA or the polynucleotide encoding the ngRNA results in incorporation of one or more intended nucleotide edits in the target gene in at least one of the cells in the population of cells. In some embodiments, introduction of the prime editor polypeptide or the polynucleotide encoding the prime editor polypeptide, the PEgRNA or the polynucleotide encoding the PEgRNA, and/or the ngRNA or the polynucleotide encoding the ngRNA results in incorporation of the one or more intended nucleotide edits in the target gene in a plurality of the population of cells. In some embodiments, introduction of the prime editor polypeptide or the poly nucleotide encoding the prime editor polypeptide, the PEgRNA or the polynucleotide encoding the PEgRNA, and/or the ngRNA or the polynucleotide encoding the ngRNA results in incorporation of the one or more intended nucleotide edits in the target gene in each cell of the population of cells. In some embodiments, introduction of the prime editor polypeptide or the polynucleotide encoding the prime editor polypeptide, the PEgRNA or the polynucleotide encoding the PEgRNA, and/or the ngRNA or the polynucleotide encoding the ngRNA results in incorporation of the one or more intended nucleotide edits in the target gene in sufficient number of cells such that the disease or disorder is treated, prevented or ameliorated.

In some embodiments, editing efficiency of the prime editing compositions and method described herein can be measured by calculating the percentage of edited target genes in a population of cells introduced with the prime editing composition. In some embodiments, the editing efficiency is determined after 1 hour, 2 hours, 6 hours, 12 hours, 24 hours, 36 hours, 48 hours, 3 days, 4 days, 5 days, 7 days. 10 days, or 14 days of exposing a target gene (e.g., a ATP7B gene within the genome of a cell) to a prime editing composition. In some embodiments, the population of cells introduced with the prime editing composition is ex vivo. In some embodiments, the population of cells introduced with the prime editing composition is in vitro. In some embodiments, the population of cells introduced with the prime editing composition is in vivo. In some embodiments, the prime editing methods disclosed herein have an editing efficiency of at least about 1%, at least about 5%, at least about 10%, at least about 20%, at least about 30%, at least about 40%, at least about 50%, at least about 60%, at least about 70%, at least about 80%, at least about 90%, at least about 95%, or at least about 99% relative to a suitable control. In some embodiments, the prime editing methods disclosed herein have an editing efficiency of at least 25% relative to a suitable control. In some embodiments, the prime editing methods disclosed herein have an editing efficiency of at least 35% relative to a suitable control, prime editing method disclosed herein has an editing efficiency of at least 30% relative to a suitable control. In some embodiments, the prime editing methods disclosed herein have an editing efficiency of at least 45% relative to a suitable control. In some embodiments, the prime editing methods disclosed herein have an editing efficiency of at least 50% relative to a suitable control.

In some embodiments, the methods disclosed herein have an editing efficiency of at least about 1%, at least about 5%, at least about 7.5%, at least about 10%, at least about 15%, at least about 20%, at least about 30%, at least about 40%, at least about 50%, at least about 60%, at least about 70%, at least about 80%, at least about 90%, or at least about 95% of editing a primary cell relative to a suitable control.

In some embodiments, the methods disclosed herein have an editing efficiency of at least about 5%, at least about 7.5%, at least about 10%, at least about 15%, at least about 20%, at least about 30%, at least about 40%, at least about 50%, at least about 60%, at least about 70%, at least about 80%, at least about 90%, or at least about 95% of editing a hepatocyte relative to a corresponding control hepatocyte. In some embodiments, the hepatocyte is a human hepatocyte.

In some embodiments, the prime editing compositions provided herein are capable of incorporated one or more intended nucleotide edits without generating a significant proportion of indels. The term “indel(s)”, as used herein, refers to the insertion or deletion of a nucleotide base within a polynucleotide, for example, a target gene. Such insertions or deletions can lead to frame shift mutations within a coding region of a gene. Indel frequency of editing can be calculated by methods known in the art. In some embodiments, indel frequency can be calculated based on sequence alignment such as the CRISPResso 2 algorithm as described in Clement et al., Nat. Biotechnol. 37(3): 224-226 (2019), which is incorporated herein in its entirety. In some embodiments, the methods disclosed herein can have an indel frequency of less than 20%, less than 10%, less than 9%, less than 8%, less than 7%, less than 6%, less than 5%, less than 4%, less than 3%, less than 2%, less than 1.5%, or less than 1%. In some embodiments, any number of indels is determined after at least 1 hour, at least 2 hours, at least 6 hours, at least 12 hours, at least 24 hours, at least 36 hours, at least 48 hours, at least 3 days, at least 4 days, at least 5 days, at least 7 days, at least 10 days, or at least 14 days of exposing a target gene (e.g., a ATP7B gene within the genome of a cell) to a prime editing composition.

In some embodiments, the prime editing compositions provided herein are capable of incorporated one or more intended nucleotide edits efficiently without generating a significant proportion of indels. In some embodiments, the prime editing methods disclosed herein have an editing efficiency of at least about 1% and an indel frequency of less than 1% in a target cell, e.g., a human primary cell or hepatocyte. In some embodiments, the prime editing methods disclosed herein have an editing efficiency of at least about 1% and an indel frequency of less than 0.5% in a target cell, e.g., a human primary cell or hepatocyte. In some embodiments, the prime editing methods disclosed herein have an editing efficiency of at least about 1% and an indel frequency of less than 0.1% in a target cell, e.g., a human primary cell or hepatocyte. In some embodiments, the prime editing methods disclosed herein have an editing efficiency of at least about 5% and an indel frequency of less than 1% in a target cell, e.g., a human primary cell or hepatocyte. In some embodiments, the prime editing methods disclosed herein have an editing efficiency of at least about 5% and an indel frequency of less than 0.5% in a target cell, e.g., a human primary cell or hepatocyte. In some embodiments, the prime editing methods disclosed herein have an editing efficiency of at least about 5% and an indel frequency of less than 0.1% in a target cell, e.g., a human primary cell or hepatocyte.

In some embodiments, the prime editing methods disclosed herein have an editing efficiency of at least about 7.5% and an indel frequency of less than 1% in a target cell, e.g., a human primary cell or hepatocyte. In some embodiments, the prime editing methods disclosed herein have an editing efficiency of at least about 7.5% and an indel frequency of less than 0.5% in a target cell, e.g., a human primary cell or hepatocyte. In some embodiments, the prime editing methods disclosed herein have an editing efficiency of at least about 7.5% and an indel frequency of less than 0.1% in a target cell, e.g., a human primary cell or hepatocyte.

In some embodiments, the prime editing methods disclosed herein have an editing efficiency of at least about 10% and an indel frequency of less than 1% in a target cell, e.g., a human primary cell or hepatocyte. In some embodiments, the prime editing methods disclosed herein have an editing efficiency of at least about 10% and an indel frequency of less than 0.5% in a target cell, e.g., a human primary cell or hepatocyte. In some embodiments, the prime editing methods disclosed herein have an editing efficiency of at least about 10% and an indel frequency of less than 0.1% in a target cell, e.g., a human primary cell or hepatocyte.

In some embodiments, the prime editing methods disclosed herein have an editing efficiency of at least about 15% and an indel frequency of less than 1% in a target cell, e.g., a human primary cell or hepatocyte. In some embodiments, the prime editing methods disclosed herein have an editing efficiency of at least about 15% and an indel frequency of less than 0.5% in a target cell, e.g., a human primary cell or hepatocyte. In some embodiments, the prime editing methods disclosed herein have an editing efficiency of at least about 15% and an indel frequency of less than 0.1% in a target cell, e.g., a human primary cell or hepatocyte.

In some embodiments, the prime editing methods disclosed herein have an editing efficiency of at least about 20% and an indel frequency of less than 1% in a target cell, e.g., a human primary cell or hepatocyte. In some embodiments, the prime editing methods disclosed herein have an editing efficiency of at least about 20% and an indel frequency of less than 0.5% in a target cell, e.g., a human primary cell or hepatocyte. In some embodiments, the prime editing methods disclosed herein have an editing efficiency of at least about 20% and an indel frequency of less than 0.1% in a target cell, e.g., a human primary cell or hepatocyte.

In some embodiments, the prime editing methods disclosed herein have an editing efficiency of at least about 30% and an indel frequency of less than 1% in a target cell, e.g., a human primary cell or hepatocyte. In some embodiments, the prime editing methods disclosed herein have an editing efficiency of at least about 30% and an indel frequency of less than 0.5% in a target cell, e.g., a human primary cell or hepatocyte. In some embodiments, the prime editing methods disclosed herein have an editing efficiency of at least about 30% and an indel frequency of less than 0.1% in a target cell, e.g., a human primary cell or hepatocyte.

In some embodiments, the prime editing methods disclosed herein have an editing efficiency of at least about 40% and an indel frequency of less than 1% in a target cell, e.g., a human primary cell or hepatocyte. In some embodiments, the prime editing methods disclosed herein have an editing efficiency of at least about 40% and an indel frequency of less than 0.5% in a target cell, e.g., a human primary cell or hepatocyte. In some embodiments, the prime editing methods disclosed herein have an editing efficiency of at least about 40% and an indel frequency of less than 0.1% in a target cell, e.g., a human primary cell or hepatocyte.

In some embodiments, the prime editing methods disclosed herein have an editing efficiency of at least about 50% and an indel frequency of less than 1% in a target cell, e.g., a human primary cell or hepatocyte. In some embodiments, the prime editing methods disclosed herein have an editing efficiency of at least about 50% and an indel frequency of less than 0.5% in a target cell, e.g., a human primary cell or hepatocyte. In some embodiments, the prime editing methods disclosed herein have an editing efficiency of at least about 50% and an indel frequency of less than 0.1% in a target cell, e.g., a human primary cell or hepatocyte.

In some embodiments, the prime editing methods disclosed herein have an editing efficiency of at least about 60% and an indel frequency of less than 1% in a target cell, e.g., a human primary cell or hepatocyte. In some embodiments, the prime editing methods disclosed herein have an editing efficiency of at least about 60% and an indel frequency of less than 0.5% in a target cell, e.g., a human primary cell or hepatocyte. In some embodiments, the prime editing methods disclosed herein have an editing efficiency of at least about 60% and an indel frequency of less than 0.1% in a target cell, e.g., a human primary cell or hepatocyte.

In some embodiments, the prime editing methods disclosed herein have an editing efficiency of at least about 70% and an indel frequency of less than 1% in a target cell, e.g., a human primary cell or hepatocyte. In some embodiments, the prime editing methods disclosed herein have an editing efficiency of at least about 70% and an indel frequency of less than 0.5% in a target cell, e.g., a human primary cell or hepatocyte. In some embodiments, the prime editing methods disclosed herein have an editing efficiency of at least about 70% and an indel frequency of less than 0.1% in a target cell, e.g., a human primary cell or hepatocyte.

In some embodiments, the prime editing methods disclosed herein have an editing efficiency of at least about 80% and an indel frequency of less than 1% in a target cell, e.g., a human primary cell or hepatocyte. In some embodiments, the prime editing methods disclosed herein have an editing efficiency of at least about 80% and an indel frequency of less than 0.5% in a target cell, e.g., a human primary cell or hepatocyte. In some embodiments, the prime editing methods disclosed herein have an editing efficiency of at least about 80% and an indel frequency of less than 0.1% in a target cell, e.g., a human primary cell or hepatocyte.

In some embodiments, the prime editing methods disclosed herein have an editing efficiency of at least about 90% and an indel frequency of less than 1% in a target cell, e.g., a human primary cell or hepatocyte. In some embodiments, the prime editing methods disclosed herein have an editing efficiency of at least about 90% and an indel frequency of less than 0.5% in a target cell, e.g., a human primary cell or hepatocyte. In some embodiments, the prime editing methods disclosed herein have an editing efficiency of at least about 90% and an indel frequency of less than 0.1% in a target cell, e.g., a human primary cell or hepatocyte.

In some embodiments, the prime editing methods disclosed herein have an editing efficiency of at least about 95% and an indel frequency of less than 1% in a target cell, e.g., a human primary cell or hepatocyte. In some embodiments, the prime editing methods disclosed herein have an editing efficiency of at least about 95% and an indel frequency of less than 0.5% in a target cell, e.g., a human primary cell or hepatocyte. In some embodiments, the prime editing methods disclosed herein have an editing efficiency of at least about 95% and an indel frequency of less than 0.1% in a target cell, e.g., a human primary cell or hepatocyte. In some embodiments, any number of indels is determined after at least 1 hour, at least 2 hours, at least 6 hours, at least 12 hours, at least 24 hours, at least 36 hours, at least 48 hours, at least 3 days, at least 4 days, at least 5 days, at least 7 days, at least 10 days, or at least 14 days of exposing a target gene (e.g., a ATP7B gene within the genome of a cell) to a prime editing composition. In some embodiments, the editing efficiency is determined after 1 hour, 2 hours, 6 hours, 12 hours, 24 hours, 36 hours, 48 hours, 3 days, 4 days, 5 days, 7 days, 10 days, or 14 days of exposing a target gene (e.g., a ATP7B gene within the genome of a cell) to a prime editing composition.

In some embodiments, the prime editing composition described herein result in less than 50%, less than 40%, less than 30%, less than 20%, less than 19%, less than 18%, less than 17%, less than 16%, less than 15%, less than 14%, less than 13%, less than 12%, less than 11%, less than 10%, less than 9%, less than 8%, less than 7%, less than 6%, less than 5%, less than 4%, less than 3%, less than 2%, less than 1%, less than 0.9%, less than 0.8%, less than 0.7%, less than 0.6%, less than 0.5%, less than 0.4%, less than 0.3%, less than 0.2%, less than 0.1%, less than 0.09%, less than 0.08%, less than 0.07%, less than 0.06%, less than 0.05%, less than 0.04%, less than 0.03%, less than 0.02%, or less than 0.01% off-target editing in a chromosome that includes the target gene. In some embodiments, off-target editing is determined after at least 1 hour, at least 2 hours, at least 6 hours, at least 12 hours, at least 24 hours, at least 36 hours, at least 48 hours, at least 3 days, at least 4 days, at least 5 days, at least 7 days, at least 10 days, or at least 14 days of exposing a target gene (e.g., a nucleic acid within the genome of a cell) to a prime editing composition.

In some embodiments, the prime editing compositions (e.g., PEgRNAs and prime editors as described herein) and prime editing; methods disclosed herein can be used to edit a target ATP7B gene. In some embodiments, the target ATP7B gene comprises a mutation compared to a wild type ATP7B gene. In some embodiments, the mutation is associated with Wilson's disease. In some embodiments, the target ATP7B gene comprises an editing target sequence that contains the mutation associated with Wilson's disease. In some embodiments, the mutation is in a coding region of the target ATP7B gene. In some embodiments, the mutation is in an exon of the target ATP7B gene. In some embodiments, the mutation is in exon 1, exon 2, exon 3, exon 4, exon 5, exon 6, exon 7, exon 8, exon 9, exon 10, exon 11, exon 12, exon 13, exon 14, exon 15, exon 16, exon 17, exon 18, exon 19, exon 20, or exon 21 of the ATP7B gene as compared to a wild type ATP7B gene. In some embodiments, the mutation is exon 8, exon 13, exon 14, exon 15, or exon 17 of the ATP71B gene as compared to a wild type ATP7B gene. In some embodiments, the mutation is in exon 3 of the ATP7B gene as compared to a wild type ATP7B gene. In some embodiments, the mutation is located in exon 8 of the ATP7B gene as compared to a wild type ATP7B gene. In some embodiments, the mutation is not a c.1288dup duplication. In some embodiments, the mutation is in exon 14 of the target ATP7B gene. In some embodiments, the mutation is located between positions 51944045 and 51944245 of human chromosome 13 as set forth in human genome assembly consortium Human build 38 (GRCh38), GenBank accession GCA_000001405.15. In some embodiments, the mutation encodes an amino acid substitution H1069Q relative to a wild type ATP7B polypeptide set forth in SEQ ID NO: 5861. In some embodiments, the editing target sequence comprises a C>A mutation at position 51944145 in human chromosome 13 as set forth in human genome assembly consortium Human build 38 (GRCh38), GenBank accession GCA_000001405.15. In some embodiments, the prime editing method comprises contacting a target ATP7B gene with a prime editing composition comprising a prime editor, a PEgRNA, and/or a ngRNA. In some embodiments, contacting the target ATP7B gene with the prime editing composition results in incorporation of one or more intended nucleotide edits in the target ATP7B gene. In some embodiments, the incorporation is in a region of the target ATP7B gene that corresponds to an editing target sequence inn the ATP7B gene. In some embodiments, the one or more intended nucleotide edits comprises a single nucleotide substitution, an insertion, a deletion, or any combination thereof, compared to the endogenous sequence of the target ATP7B gene. In some embodiments, incorporation of the one or more intended nucleotide edits results in replacement of one or more mutations with the corresponding sequence that encodes a wild type ATP7B polypeptide set forth in SEQ ID NO: 5861. In some embodiments, incorporation of the one or more intended nucleotide edits results in replacement of the one or more mutations with the corresponding sequence in a wild type ATP7B gene. In some embodiments, incorporation of the one more intended nucleotide edits results in correction of a mutation in the target ATP7B gene. In some embodiments, the target A P7B1 gene comprises an editing template sequence that contains the mutation. In some embodiments, contacting the target ATP7B gene with the prime editing composition results in incorporation of one or more intended nucleotide edits in the target ATP7B gene, which corrects the mutation in the editing target sequence (or a double stranded region comprising the editing target sequence and the complementary sequence to the editing target sequence on a target strand) in the target ATP73 gene.

In some embodiments, incorporation of the one more intended nucleotide edits results in correction of a mutation in exon 14 of the target ATP7B gene as compared to a wild type ATP7B gene. In some embodiments, incorporation of the one more intended nucleotide edits results in correction of a mutation located between positions 51944045 and 51944245 of human chromosome 13 in the target ATP7B gene as set forth in human genome assembly consortium Human build 38 (GRCh38), GenBank accession GCA_000001405.15. In some embodiments, incorporation of the one more intended nucleotide edits results in an A>C nucleotide substitution at position 51944145 in human chromosome 13 in the target ATP7B gene as compared to the endogenous sequence of the target ATP7B gene, thereby correcting a C>A mutation at position 51944145 in human chromosome 13 in the target ATP7B gene as set forth in human genome assembly consortium Human build 38 (GRCh38), GenBank accession GCA_000001405.15. In some embodiments, incorporation of the one more intended nucleotide edits results in correction of an ATP7B gene sequence that encodes a H1069Q amino acid substitution, and restores wild type expression and function of the ATP7B protein.

In some embodiments, the target ATP7B gene is in a target cell. Accordingly, in one aspect provided herein is a method of editing a target cell comprising a target ATP7B gene that encodes a polypeptide that comprises one or more mutations relative to a wild type ATP7B gene. In some embodiments, the methods of the present disclosure comprise introducing a prime editing composition comprising a PEgRNA, a prime editor polypeptide, and/or a ngRNA into the target cell that has the target ATP7B gene to edit the target ATP7B gene, thereby generating an edited cell. In some embodiments, the target cell is a mammalian cell. In some embodiments, the target cell is a human cell. In some embodiments, the target cell is a primary cell. In some embodiments, the target cell is a human primary cell. In some embodiments, the target cell is a progenitor cell. In some embodiments, the target cell is a human progenitor cell. In some embodiments, the target cell is a stein cell. In some embodiments, the target cell is a human stem cell. In some embodiments, the target cell is a hepatocyte. In some embodiments, the target cell is a human hepatocyte. In some embodiments, the target cell is a primary human hepatocyte derived from an induced human pluripotent stem cell (iPSC). In some embodiments, the cell is a neuron. In some embodiments, the cell is a neuron from basal ganglia. In some embodiments, the cell is a neuron from basal ganglia of a subject. In some embodiments, the cell is a neuron in the basal ganglia of a subject.

In some embodiments, components of a prime editing composition described herein are provided to a target cell in vitro. In some embodiments, components of a prime editing composition described herein are provided to a target cell ex vivo. In some embodiments, components of a prime editing composition described herein are provided to a target cell in vivo.

In some embodiments, incorporation of the one or more intended nucleotide edits in the target ATP7B gene that comprises one or more mutations restores wild type expression and function of the ATP7B protein encoded by the ATP7B gene. In some embodiments, the target ATP7 gene encodes a H1069Q amino acid substitution as compared to the wild type ATP7B protein prior to incorporation of the one or more intended nucleotide edits. In some embodiments, expression and/or function of the ATP7B protein may be measured when expressed in a target cell. In some embodiments, incorporation of the one or more intended nucleotide edits in the target ATP7B gene comprising one or more mutations lead to a fold change in a level of ATP7B gene expression, ATP7B protein expression, or a combination thereof. In some embodiments, a change in the level of ATP7B expression level can comprise a fold change of, e.g., 2-fold, 3-fold, 4-fold, 5-fold, 6-fold, 7-fold, 8-fold, 9-fold. 10-fold, 15-fold, 20-fold, 25-fold, 30-fold. 40-fold, 50-fold. 60-fold, 70-fold, 80-fold, 90-fold, 100-fold or greater as compared to expression in a suitable control cell not introduced with a prime editing composition described herein. In some embodiments, incorporation of the one or more intended nucleotide edits in the target ATP7B gene that comprises one or more mutations restores wild type expression of the ATP7B protein by at least 10%, 20%, 30%, 40%, 50%, 60%, 70%, 80%. 90% 95%, o99% or more as compared to wild type expression of the ATP7B protein in a suitable control cell that comprises a wild type ATP7B gene.

In some embodiments, an ATP7B expression increase can be measured by a functional assay. In some embodiments, the functional assay can comprise a copper sensitivity assay, a cell viability assay, or a combination thereof. In some embodiments, protein expression can be measured using a protein assay. In some embodiments, protein expression can be measured using antibody testing. In some embodiments, an antibody can comprise anti-ATP7B. In some embodiments, protein expression can be measured using ELISA, mass spectrometry, Western blot, sodium dodecyl sulfate polyacrylamide gel electrophoresis (SDS-PAGE), high performance liquid chromatography (HPLC), electrophoresis, or any combination thereof. In some embodiments, a protein assay can comprise SDS-PAGE and densitometric analysis of a Coomassie Blue-stained gel. In some embodiments, ATP7B activity can be measured by measuring ATPase activity. In some embodiments, ATPase activity can be measured using an ATPase assay.

### Methods of Treating Wilson's Disease

In some embodiments, provided herein are methods for treatment of a subject diagnosed with a disease associated with or caused by one or more pathogenic mutations that can be corrected by prime editing. In some embodiments, provided herein are methods for treating Wilson's disease that comprise administering to a subject a therapeutically effective amount of a prime editing composition, or a pharmaceutical composition comprising a prime editing composition as described herein. In some embodiments, administration of the prime editing composition results in incorporation of one or more intended nucleotide edits in the target gene in the subject. In some embodiments, administration of the prime editing composition results in correction of one or more pathogenic mutations, e.g. point mutations, insertions, or deletions, associated with Wilson's disease in the subject. In some embodiments, the target gene comprise an editing target sequence that contains the pathogenic mutation. In some embodiments, administration of the prime editing composition results in incorporation of one or more intended nucleotide edits in the target gene that corrects the pathogenic mutation in the editing target sequence (or a double stranded region comprising the editing target sequence and the complementary sequence to the editing target sequence on a target strand) of the target gene in the subject.

In some embodiments, the method provided herein comprises administering to a subject an effective amount of a prime editing composition, for example, a PEgRNA, a prime editor, and/or a ngRNA. In some embodiments, the method comprises administering to the subject an effective amount of a prime editing composition described herein, for example, polynucleotides, vectors, or constructs that encode prime editing composition components, or RNPs, LNPs, and/or polypeptides comprising prime editing composition components. Prime editing compositions can be administered to target the ATP7B gene in a subject, e.g., a human subject, suffering from, having, susceptible to, or at risk for Wilsons' disease. Identifying a subject in need of such treatment can be in the judgment of a subject or a health care professional and can be subjective (e.g. opinion) or objective (e.g. measurable by a test or diagnostic method). In some embodiments, the subject has Wilson's disease.

In some embodiments, the subject has been diagnosed with Wilson's disease by sequencing of a ATP7B gene in the subject. In some embodiments, the subject comprises at least a copy of ATP7B gene that comprises one or more mutations compared to a wild type ATP7B gene. In some embodiments, the subject comprises at least a copy of ATP7B gene that comprises a mutation in a coding region of the ATP7B gene. In some embodiments, the subject comprises at least a copy of ATP7B gene that comprises a mutation in exon 1, exon 2, exon 3, exon 4, exon 5, exon 6, exon 7, exon 8, exon 9, exon 10, exon 11, exon 12, exon 13, exon 14, exon 15, exon 16, exon 17, exon 18, exon 19, exon 20, or exon 21, as compared to a wild type ATP7B gene. In some embodiments, the subject comprises at least a copy of ATP7B gene that comprises a mutation in exon 8, exon 13, exon 14, exon 15, or exon 17 as compared to a wild type ATP7B gene. In some embodiments, the subject comprises at least a copy of ATP7B gene that comprises a mutation in exon 14 of the ATP7B gene as compared to a wild type ATP7B gene. In some embodiments, the subject comprises at least a copy of ATP7B gene that comprises a mutation in exon 3 as compared to a wild type ATP7B gene. In some embodiments, the mutation is not a c.1288dup duplication. In some embodiments, the subject comprises at least a copy of ATP7B gene that encodes a polypeptide that comprises an amino acid substitution H1069Q relative to a wild type ATP7B polypeptide set forth in SEQ ID NO: 5861.

In some embodiments, the method comprises directly administering prime editing compositions provided herein to a subject. The prime editing compositions described herein can be delivered with in any form as described herein, e.g., as LNPs, RNPs, polynucleotide vectors such as viral vectors, or mRNAs. The prime editing compositions can be formulated with any pharmaceutically acceptable carrier described herein or known in the art for administering directly to a subject. Components of a prime editing composition or a pharmaceutical composition thereof may be administered to the subject simultaneously or sequentially. For example, in some embodiments, the method comprises administering a prime editing composition, or pharmaceutical composition thereof, comprising a complex that comprises a prime editor fusion protein and a PEgRNA and/or a ngRNA, to a subject. In some embodiments, the method comprises administering a polynucleotide or vector encoding a prime editor to a subject simultaneously with a PEgRNA and/or a ngRNA. In some embodiments, the method comprises administering a polynucleotide or vector encoding a prime editor to a subject before administration with a PEgRNA and/or a ngRNA.

Suitable routes of administrating the prime editing compositions to a subject include, without limitation: topical, subcutaneous, transdermal, intradermal, intralesional, intraarticular, intraperitoneal, intravesical, transmucosal, gingival, intradental, intracochlear, transtympanic, intraorgan, epidural, intrathecal, intramuscular, intravenous, intravascular, intraosseus, periocular, intratumoral, intracerebral, and intracerebroventricular administration. In some embodiments, the compositions described are administered intraperitoneally, intravenously, or by direct injection or direct infusion. In some embodiments, the compositions described are administered by direct injection or infusion into the liver of a subject. In some embodiments, the compositions described herein are administered to a subject by injection, by means of a catheter, by means of a suppository, or by means of an implant.

In some embodiments, the method comprises administering cells edited with a prime editing composition described herein to a subject. In some embodiments, the cells are allogeneic. In some embodiments, allogeneic cells are or have been contacted ex vivo with a prime editing composition or pharmaceutical composition thereof and are introduced into a human subject in need thereof. In some embodiments, the cells are autologous to the subject. In some embodiments, cells are removed from a subject and contacted ex vivo with a prime editing composition or pharmaceutical composition thereof and are re-introduced into the subject.

In some embodiments, cells are contacted ex vivo with one or more components of a prime editing composition. In some embodiments, the ex vivo-contacted cells are introduced into the subject, and the subject is administered in vivo with one or more components of a prime editing composition. For example, in some embodiments, cells are contacted ex vivo with a prime editor and introduced into a subject. In some embodiments, the subject is then administered with a PEgRNA and/or a ngRNA, or a polynucleotide encoding the PEgRNA and/or the ngRNA.

In some embodiments, cells contacted with the prime editing composition are determined for incorporation of the one or more intended nucleotide edits in the genome before re-introduction into the subject. In some embodiments, the cells are enriched for incorporation of the one or more intended nucleotide edits in the genome before re-introduction into the subject. In some embodiments, the edited cells are primary cells. In some embodiments, the edited cells are progenitor cells. In some embodiments, the edited cells are stem cells. In some embodiments, the edited cells are hepatocytes. In some embodiments, the edited cells are primary human cells. In some embodiments, the edited cells are human progenitor cells. In some embodiments, the edited cells are human stem cells. In some embodiments, the edited cells are human hepatocytes. In some embodiments, the cell is a neuron. In some embodiments, the cell is a neuron from basal ganglia. In some embodiments, the cell is a neuron from basal ganglia of a subject. In some embodiments, the cell is a neuron in the basal ganglia of a subject. The prime editing composition or components thereof may be introduced into a cell by any delivery approaches as described herein, including LNP administration, RNP administration, electroporation, nucleofection, transfection, viral transduction, microinjection, cell membrane disruption and diffusion, or any other approach known in the art.

The cells edited with prime editing can be introduced into the subject by any route known in the art. In some embodiments, the edited cells are administered to a subject by direct infusion. In some embodiments, the edited cells are administered to a subject by intravenous infusion. In some embodiments, the edited cells are administered to a subject as implants.

The pharmaceutical compositions, prime editing compositions, and cells, as described herein, can be administered in effective amounts. In some embodiments, the effective amount depends upon the mode of administration. In some embodiments, the effective amount depends upon the stage of the condition, the age and physical condition of the subject, the nature of concurrent therapy, if any, and like factors well-known to the medical practitioner.

The specific dose administered can be a uniform dose for each subject. Alternatively, a subject's dose can be tailored to the approximate body weight of the subject. Other factors in determining the appropriate dosage can include the disease or condition to be treated or prevented, the severity of the disease, the route of administration, and the age, sex and medical condition of the patient.

In embodiments wherein components of a prime editing composition are administered sequentially, the time between sequential administration can be at least 1 hour, at least 2 hours, at least 6 hours, at least 12 hours, at least 24 hours, at least 36 hours, at least 48 hours, at least 3 days, at least 4 days, at least 5 days, at least 7 days, at least 10 days, or at least 14 days.

In some embodiments, a method of monitoring treatment progress is provided. In some embodiments, the method includes the step of determining a level of diagnostic marker, for example, correction of a mutation in ATP7B gene, or diagnostic measurement associated with Wilson's disease, (e.g., copper sensitivity screen or assay) in a subject suffering from Wilson's disease symptoms and has been administered an effective amount of a prime editing composition described herein. The level of the diagnostic marker determined in the method can be compared to known levels of the marker in either healthy normal controls or in other afflicted subjects to establish the subject's disease status.

### Delivery

Prime editing compositions described herein can be delivered to a cellular environment with any approach known in the art. Components of a prime editing composition can be delivered to a cell by the same mode or different modes. For example, in some embodiments, a prime editor can be delivered as a polypeptide or a polynucleotide (DNA or RNA) encoding the polypeptide. In some embodiments, a PEgRNA can be delivered directly as an RNA or as a DNA encoding the PEgRNA.

In some embodiments, a prime editing composition component is encoded by a polynucleotide, a vector, or a construct. In some embodiments, a prime editor polypeptide, a PEgRNA and/or a ngRNA is encoded by a polynucleotide. In some embodiments, the polynucleotide encodes a prime editor fusion protein comprising a DNA binding domain and a DNA polymerase domain. In some embodiments, the polynucleotide encodes a DNA polymerase domain of a prime editor. In some embodiments, the polynucleotide encodes a DNA polymerase domain of a prime editor. In some embodiments, the polynucleotide encodes a portion of a prime editor protein, for example, a N-terminal portion of a prime editor fusion protein connected to an intein-N. In some embodiments, the polynucleotide encodes a portion of a prime editor protein, for example, a C-terminal portion of a prime editor fusion protein connected to an intein-C. In some embodiments, the polynucleotide encodes a PEgRNA and/or a ngRNA. In some embodiments, the polypeptide encodes two or more components of a prime editing composition, for example, a prime editor fusion protein and a PEgRNA.

In some embodiments, the polynucleotide encoding one or more prime editing composition components is delivered to a target cell is integrated into the genome of the cell for long-term expression, for example, by a retroviral vector. In some embodiments, the polynucleotide delivered to a target cell is expressed transiently. For example, the polynucleotide may be delivered in the form of a mRNA, or a non-integrating vector (non-integrating virus, plasmids, minicircle DNAs) for episomal expression.

In some embodiments, a polynucleotide encoding one or more prime editing system components can be operably linked to a regulatory element, e.g., a transcriptional control element, such as a promoter. In some embodiments, the polynucleotide is operably linked to multiple control elements. Depending on the expression system utilized, any of a number of suitable transcription and translation control elements, including constitutive and inducible promoters, transcription enhancer elements, transcription terminators, etc. may be used in the expression vector (e.g., U6 promoter, H1 promoter).

In some embodiments, the polynucleotide encoding one or more prime editing composition components is a part of, or is encoded by, a vector. In some embodiments, the vector is a viral vector. In some embodiments, the vector is a non-viral vector.

Non-viral vector delivery systems can include DNA plasmids. RNA (e.g., a transcript of a vector described herein), naked nucleic acid, and nucleic acid complexed with a delivery vehicle, such as a liposome. In some embodiments, the polynucleotide is provided as an RNA, e.g., a mRNA or a transcript. Any RNA of the prime editing systems, for example a guide RNA or a base editor-encoding mRNA, can be delivered in the form of RNA. In some embodiments, one or more components of the prime editing system that are RNAs is produced by direct chemical synthesis or may be transcribed in vitro from a DNA. In some embodiments, a mRNA that encodes a prime editor polypeptide is generated using ii vitro transcription. Guide polynucleotides (e.g., PEgRNA or ngRNA) can also be transcribed using in vitro transcription from a cassette containing a T7 promoter, followed by the sequence “GG”, and guide polynucleotide sequence. In some embodiments, the prime editor encoding mRNA. PEgRNA, and/or ngRNA are synthesized in vitro using an RNA polymerase enzyme (e.g., T7 polymerase, T3 polymerase, SP6 polymerase, etc.). Once synthesized, the RNA can directly contact a target ATP7B gene or can be introduced into a cell using any suitable technique for introducing nucleic acids into cells (e.g., microinjection, electroporation, transfection). In some embodiments, the prime editor-coding sequences, the PEgRNAs, and/or the ngRNAs are modified to include one or more modified nucleoside e.g. using pseudo-U or 5-Methyl-C.

Methods of non-viral delivery of nucleic acids can include lipofection, nucleofection, microinjection, biolistics, virosomes, liposomes, immunoliposomes, polycation or lipid:nucleic acid conjugates, naked DNA, artificial virions, cell membrane disruption by a microfluidics device, and agent-enhanced uptake of DNA. Cationic and neutral lipids that are suitable for efficient receptor-recognition lipofection of polynucleotides can be used. Delivery can be to cells (e.g., in vitro or ex vivo administration) or target tissues (e.g., in vivo administration). The preparation of lipid:nucleic acid complexes, including targeted liposomes such as immunolipid complexes, can be used.

Viral vector delivery systems can include DNA and RNA viruses, which can have either episomal or integrated genomes after delivery to the cell. RNA or DNA viral based systems can be used to target specific cells and trafficking the viral payload to an organelle of the cell. Viral vectors can be administered directly (in vivo) or they can be used to treat cells in vitro, and the modified cells can optionally be administered (ex vivo).

In some embodiments, the viral vector is a retroviral, lentiviral, adenoviral, adeno-associated viral or herpes simplex viral vector. Retroviral vectors can include those based upon murine leukemia virus (MuLV), gibbon ape leukemia virus (GaLV), Simian Immuno deficiency virus (SIV), human immuno deficiency virus (HIV), and combinations thereof. In some embodiments, the retroviral vector is a lentiviral vector. In some embodiments, the retroviral vector is a gamma retroviral vector. In some embodiments, the viral vector is an adenoviral vector. In some embodiments, the viral vector is an adeno-associated virus (“AAV”) vector.

In some embodiments, polynucleotides encoding one or more prime editing composition components are packaged in a virus particle. Packaging cells can be used to form virus particles that can infect a target cell. Such cells can include 293 cells, (e.g., for packaging adenovirus), and .psi.2 cells or PA317 cells (e.g., for packaging retrovirus). Viral vectors can be generated by producing a cell line that packages a nucleic acid vector into a viral particle. The vectors can contain the minimal viral sequences required for packaging and subsequent integration into a host. The vectors can contain other viral sequences being replaced by an expression cassette for the polynucleotide(s) to be expressed. The missing viral functions can be supplied in trans by the packaging cell line. For example, AAV vectors can comprise ITR sequences from the AAV genome which are required for packaging and integration into the host genome.

In some embodiments, dual AAV vectors are generated by splitting a large transgene expression cassette in two separate halves (5′ and 3′ ends that encode N-terminal portion and C-terminal portion of, e.g., a prime editor polypeptide), where each half of the cassette is no more than 5 kb in length, optionally no more than 4.7 kb in length, and is packaged in a single AAV vector. In some embodiments, the full-length transgene expression cassette is reassembled upon co-infection of the same cell by both dual AAV vectors. In some embodiments, a portion or fragment of a prime editor polypeptide, e.g. a Cas9 nickase, is fused to an intein. The portion or fragment of the polypeptide can be fused to the N-terminus or the C-terminus of the intein. In some embodiments, a N-terminal portion of the polypeptide is fused to an intein-N, and a C-terminal portion of the polypeptide is separately fused to an intein-C. In some embodiments, a portion or fragment of a prime editor fusion protein is fused to an intein and fused to an AAV capsid protein. The intein, nuclease and capsid protein can be fused together in any arrangement (e.g., nuclease-intein-capsid, intein-nuclease-capsid, capsid-intein-nuclease, etc.). In some embodiments, a polynucleotide encoding a prime editor fusion protein is split in two separate halves, each encoding a portion of the prime editor fusion protein and separately fused to an intein. In some embodiments, each of the two halves of the polynucleotide is packaged in an individual AAV vector of a dual AAV vector system. In some embodiments, each of the two halves of the polynucleotide is no more than 5 kb in length, optionally no more than 4.7 kb in length. In some embodiments, the full-length prime editor fusion protein is reassembled upon co-infection of the same cell by both dual AAV vectors, expression of both halves of the prime editor fusion protein, and self-excision of the inteins.

A target cell can be transiently or non-transiently transfected with one or more vectors described herein. A cell can be transfected as it naturally occurs in a subject. A cell can be taken or derived from a subject and transfected. A cell can be derived from cells taken from a subject, such as a cell line. In some embodiments, a cell transfected with one or more vectors described herein can be used to establish a new cell line comprising one or more vector-derived sequences. In some embodiments, a cell transiently transfected with the compositions of the disclosure (such as by transient transfection of one or more vectors, or transfection with RNA), and modified through the activity of a prime editor, can be used to establish a new cell line comprising cells containing the modification but lacking any other exogenous sequence. Any suitable vector compatible with the host cell can be used with the methods of the disclosure. Non-limiting examples of vectors include pXT1, pSG5, pSVK3, pBPV, pMSG, and pSVLSV40.

In some embodiments, a prime editor protein can be provided to cells as a polypeptide. In some embodiments, the prime editor protein is fused to a polypeptide domain that increases solubility of the protein. In some embodiments, the prime editor protein is formulated to improve solubility of the protein.

In some embodiment, a prime editor polypeptide is fused to a polypeptide permeant domain to promote uptake by the cell. In some embodiments, the permeant domain is a including peptide, a peptidomimetic, or a non-peptide carrier. For example, a permeant peptide may be derived from the third alpha helix of Drosophila melanogaster transcription factor Antennapaedia, referred to as penetratin, which comprises the amino acid sequence RQIKIWFQNRRMKWKK (SEQ ID NO: 5897). As another example, the permeant peptide can comprise the HIV-1 tat basic region amino acid sequence, which may include, for example, amino acids 49-57 of naturally-occurring tat protein. Other permeant domains can include poly-arginine motifs, for example, the region of amino acids 34-56 of HIV-1 rev protein, nona-arginine (SEQ ID NO: 5898), and octa-arginine (SEQ ID NO: 5899). The nona-arginine (R9) sequence (SEQ ID NO: 5898) can be used. The site at which the fusion can be made may be selected in order to optimize the biological activity, secretion or binding characteristics of the polypeptide.

In some embodiments, a prime editor polypeptide is produced in vitro or by host cells, and it may be further processed by unfolding, e.g., heat denaturation, DTT reduction, etc. and may be further refolded. In some embodiments, a prime editor polypeptide is prepared by in vitro synthesis. Various commercial synthetic apparatuses can be used. By using synthesizers, naturally occurring amino acids can be substituted with unnatural amino acids. In some embodiments, a prime editor polypeptide is isolated and purified in accordance with recombinant synthesis methods, for example, by expression in a host cell and the lysate purified using HPLC, exclusion chromatography, gel electrophoresis, affinity chromatography, or other purification technique.

In some embodiments, a prime editing composition, for example, prime editor polypeptide components and PEgRNA/ngRNA are introduced to a target cell by nanoparticles. In some embodiments, the prime editor polypeptide components and the PEgRNA and/or ngRNA form a complex in the nanoparticle. Any suitable nanoparticle design can be used to deliver genome editing system components or nucleic acids encoding such components. In some embodiments, the nanoparticle is inorganic. In some embodiments, the nanoparticle is organic. In some embodiments, a prime editing composition is delivered to a target cell, e.g., a hepatocyte, in an organic nanoparticle, e.g. a lipid nanoparticle (LNP) or polymer nanoparticle.

In some embodiments, LNPs are formulated from cationic, anionic, neutral lipids, or combinations thereof. In some embodiments, neutral lipids, such as the fusogenic phospholipid DOPE or the membrane component cholesterol, are included to enhance transfection activity and nanoparticle stability. In some embodiments, LNPs are formulated with hydrophobic lipids, hydrophilic lipids, or combinations thereof. Lipids may be formulated in a wide range of molar ratios to produce an LNP. Any lipid or combination of lipids that are known in the art can be used to produce an LNP. Exemplary lipids used to produce LNPs are provided in Table 3 below.

In some embodiments, components of a prime editing composition form a complex prior to delivery to a target cell. For example, a prime editor fusion protein, a PEgRNA, and/or a ngRNA can form a complex prior to delivery to the target cell. In some embodiments, a prime editing polypeptide (e.g. a prime editor fusion protein) and a guide polynucleotide (e.g. a PEgRNA or ngRNA) form a ribonucleoprotein (RNP) for delivery to a target cell. In some embodiments, the RNP comprises a prime editor fusion protein in complex with a PEgRNA. RNPs may be delivered to cells using known methods, such as electroporation, nucleofection, or cationic lipid-mediated methods, or any other approaches known in the art. In some embodiments, delivery of a prime editing composition or complex to the target cell does not require the delivery of foreign DNA into the cell. In some embodiments, the RNP comprising the prime editing complex is degraded over time in the target cell. Exemplary lipids for use in nanoparticle formulations and/or gene transfer are shown in Table 3 below.

TABLE 3
Exemplary lipids for nanoparticle formulation or gene transfer

| Lipid | Abbreviation | Feature |
|---|---|---|
| 1,2-Dioleoyl-sn-glycero-3-phosphatidylcholine | DOPC | Helper |
| 1,2-Dioleoyl-sn-glycero-3-phosphatidylethanolamine | DOPE | Helper |
| Cholesterol | | Helper |
| N41-(2,3-Dioleyloxy)prophyliN,N,N-trimethylammonium | DOTMA | Cationic |
| chloride | | |
| 1,2-Dioleoyloxy-3-trimethylammonium-propane | DOGS | Cationic |
| Dioctadecylamidoglycylspermine | | |
| N-(3-Aminopropy1)-N,N-dimethy 1-2,3-bis(dodecyloxy)-1- | GAP-DLRIE | Cationic |
| propanaminium bromide | | |
| Cetyltrimethylammonium bromide | CTAB | Cationic |
| 6-Lauroxyhexyl omithinate | LHON | Cationic |
| 1-(2,3-Dioleoyloxypropy1)-2,4,6-trimethylpyridinium | 2Oc | Cationic |
| 2,3-Dioleyloxy-N-P(spenninecarboxamido-ethy1J-N,Ndimethyl- | DOSPA | Cationic |
| 1-propanatninium trifluoroacetate | | |
| 1,2-Dioley 1-3-trimethylamtnonium-propane | DOPA | Cationic |
| N-(2-Hydroxyethyl)-N,N-dimethy 1-2,3-bis(tetradecyloxy)-1- | MDRIE | Cationic |
| propanaminium bromide | | |
| Dimyristooxypropyl dimethyl hydroxyethyl ammonium bromide | DMRI | Cationic |
| 3β-[N-(N′,N′-Dimethylaminoethane)-carbamoyl]cholesterol | DC-Chol | Cationic |
| Bis-guanidium-tren-cholesterol | BGTC | Cationic |
| 1,3-Diodeoxy-2-(6-carboxy-spermy1)-propylamide | DOSPER | Cationic |
| Dimethyloctadecylammonium bromide | DDAB | Cationic |
| Dioctadecylamidoglicylspermidin | DSL | Cationic |
| rac-[(2,3-Dioctadecyloxypropyl)(2-hydroxyethyl)]- | CLIP-1 | Cationic |
| dimethylammonium chloride | | |
| rac-[2(2,3-Dihexadecyloxypropyloxymethyloxy) | CLIP-6 | Cationic |
| ethyl\|trimethylammoniun bromide | | |
| Ethyldimyristoylphosphatidylcholine | EDMPC | Cationic |
| 1,2-Distearyloxy-N,N-dimethyl-3-aminopropane | DSDMA | Cationic |
| 1,2-Dimyristoyl-trimethylammonium propane | DMTAP | Cationic |
| O,O′-Dimyristyl-N-lysyl aspartate | DMKE | Cationic |
| 1,2-Distearoyl-sn-glycero-3-ethylpho sphocholine | DSEPC | Cationic |
| N-Palmitoyl D-erythro-sphingosyl carbamoyl-spenmine | CCS | Cationic |
| N-t-Butyl-N0-tetradecy1-3-tetradecylaminopropionamidine | diC14-amidine | Cationic |
| Octadecenolyoxy[ethyl-2-heptadeceny1-3 hydroxyethyl] | DOTIM | Cationic |
| imidazolinium chloride | | |
| N1-Cholesteryloxycarbonyl-3,7-diazanonane-1,9-diamine | CDAN | Cationic |
| 2-(3-Bis(3-amino-propy1)-amino]propylamino)- | RPR209120 | Cationic |
| Nditetradecylcarbamoylme-ethyl-acetamide | | |
| 1,2-dilinoleyloxy-3-dimethylaminopropane | DLinDMA | Cationic |
| 2,2-dilinoley1-4-dimethylaminoethyl-[1,3]-dioxolane | DLin-KC2- | Cationic |
| | DMA | |
| dilinoleyl-methyl-4-dimethylaminobutyrate | DLin-MC3- | Cationic |
| | DMA | |

Exemplary polymers for use in nanoparticle formulations and/or gene transfer are shown in Table 4 below.

TABLE 4
Exemplary lipids for nanoparticle formulation or gene transfer

| Polymer | Abbreviation |
|---|---|
| Poly(ethylene)glycol | PEG |
| Polyethylenimine | PEI |
| Dithiobis (succinimidylpropionate) | DSP |
| Dimethyl-3,3′-dithiobispropionimidate | DTBP |
| Poly(ethyleneimine)biscarbamate | PEIC |
| Poly(L-lysine) | PLL |
| Histidine modified PLL | |
| Poly(N-vinylpyrrolidone) | PVP |
| Poly(propylenimine) | PPI |
| Poly(amidoamine) | PAMAM |
| Poly(amidoethylenimine) | SS_PAEI |
| Triethylenetetramine | TETA |
| Poly(β-aminoester) | |
| Poly(4-hydroxy-L-proline ester) | PHP |
| Poly(allylamine) | |
| Poly(α-[4-aminobutyl]-L-glycolic acid) | PAGA |
| Poly(D,L-lactic-co-glycolic acid) | PLGA |
| Poly(N-ethyl-4-vinylpyridinium bromide) | |
| Poly(phosphazene)s | PPZ |
| Poly(phosphoester)s | PPE |
| Poly(phosphoramidate)s | PPA |
| Poly(N-2-hydroxypropylmethacrylamide) | pHPMA |
| Poly (2-(dimethylamino)ethyl methacrylate) | pDMAEMA |
| Poly(2-aminoethyl propylene phosphate) | PPE-EA |
| Chitosan | |
| Galactosylated chitosan | |
| N-dodacylated chitosam | |
| Histone | |
| Collagen | |
| Dextran-spermine | D-SPM |

Exemplary delivery methods for polynucleotides encoding prime editing composition components are shown in Table 5 below.

TABLE 5
Exemplary polynucleotide delivery methods

| | | Delivery into | | | Type of |
|---|---|---|---|---|---|
| | | Non-Dividing | Duration of | Genome | Molecule |
| Delivery | Vector/Mode | Cells | Expression | Integration | Delivered |
| Physical | (e.g., | YES | Transient | NO | Nucleic Acids |
| | electroporation, | | | | and Proteins |
| | particle gun, | | | | |
| | Calcium phosphate | | | | |
| | transfection) | | | | |
| Viral | Retrovirus | NO | Stable | YES | RNA |
| | Lentivirus | YES | Stable | YES/NO with | RNA |
| | | | | modification | |
| | Adenovirus | YES | Transient | NO | DNA |
| | Adeno-Associated | YES | Stable | NO | DNA |
| | Virus (AAV) | | | | |
| | Vaccinia Virus | YES | Very Transient | NO | DNA |
| | Herpes Simplex | YES | Stable | NO | DNA |
| | Virus | | | | |
| Non-Viral | Cationic | YES | Transient | Depends on | Nucleic acids |
| | | | | what is | and Proteins |
| | | | | delivered | |
| | Polymeric | YES | Transient | NO | Nucleic Acids |
| | Nanoparticles | | | | |
| Biological | Attenuated Bacteria | YES | Transient | NO | Nucleic Acids |
| Non-Viral | Engineered | YES | Transient | NO | Nucleic Acids |
| Delivery | Bacteriophages | | | | |
| Vehicles | Mammalian Virus- | YES | Transient | NO | Nucleic Acids |
| | like Particles | | | | |
| | Biological | YES | Transient | NO | Nucleic Acids |
| | liposomes: | | | | |
| | Erythrocyte Ghosts | | | | |
| | and Exosomes | | | | |

The prime editing compositions of the disclosure, whether introduced as polynucleotides or polypeptides, can be provided to the cells for about 30 minutes to about 24 hours, e.g., 1 hour, 1.5 hours, 2 hours, 2.5 hours, 3 hours, 3.5 hours 4 hours, 5 hours, 6 hours, 7 hours, 8 hours, 12 hours, 16 hours, 18 hours, 20 hours, or any other period from about 30 minutes to about 24 hours, which can be repeated with a frequency of about every day to about every 4 days, e.g., every 1.5 days, every 2 days, every 3 days, or any other Frequency from about every day to about every four days. The compositions may be provided to the subject cells one or more times, e.g., one time, twice, three times, or more than three times, and the cells allowed to incubate with the agent(s) for some amount of time following each contacting event e.g., 16-24 hours. In cases in which two or more different prime editing system components, e.g., two different polynucleotide constructs are provided to the cell (e.g., different components of the same prim editing system, or two different guide nucleic acids that are complementary to different sequences within the same or different target genes), the compositions may be delivered simultaneously (e.g., as two polypeptides and/or nucleic acids). Alternatively, they may be provided sequentially, e.g., one composition being provided first, followed by a second composition.

The prime editing compositions and pharmaceutical compositions of the disclosure, whether introduced as polynucleotides or polypeptides, can be administered to subjects in need thereof for about 30 minutes to about 24 hours, e.g., 1 hour, 1.5 hours, 2 hours, 2.5 hours. 3 hours, 3.5 hours 4 hours, 5 hours, 6 hours, 7 hours, 8 hours, 12 hours, 16 hours, 18 hours, 20 hours, or any other period from about 30 minutes to about 24 hours, which can be repeated with a frequency of about every day to about every 4 days, e.g., every 1.5 days, every 2 days, every 3 days, or any other frequency from about every day to about every four days. The compositions may be provided to the subject one or more times, e.g., one time, twice, three times, or more than three times. In cases in which two or more different prime editing system components, e.g. two different polynucleotide constructs are administered to the subject (e.g., different components of the same prim editing system, or two different guide nucleic acids that are complementary to different sequences within the same or different target genes), the compositions may be administered simultaneously (e.g., as two polypeptides and/or nucleic acids). Alternatively, they may be provided sequentially, e.g., one composition being provided first, followed by a second composition.

### EXAMPLES

The following examples are provided for illustrative purposes only and are not intended to limit the scope of the claims provided herein.

### Example 1—General Methods

PEgRNA assembly: PEgRNA libraries are assembled by one of three methods: in the first method, pooled synthesized DNA oligos encoding the PEgRNA and flanking U6 expression plasmid homology regions are cloned into U6 expression plasmids via Gibson cloning and sequencing of bacterial colonies via Sanger or Next-generation sequencing. In the second method, double-stranded linear DNA fragments encoding PEgRNA and homology sequences as above are individually Gibson-cloned into U6 expression plasmids. In the third method, for each PEgRNA, separate oligos encoding a protospacer, a gRNA scaffold, and PEgRNA extension (PBS and RTT) are ligated, and then cloned into a U6 expression plasmid as described in Anzalone et al., Nature. 2019 December; 576(7785):149-157. Bacterial colonies carrying sequence-verified plasmids are propagated in LB or TB. Plasmid DNA is purified by minipreps for mammalian transfection.

Mammalian cell culture and transfection: HEK293T and Huh-7 cells are propagated in DMEM with 10% FBS. HepG2 cells are propagated in EMEM with 10% FBS. Cells are seeded in 96-well plates and then transfected with Lipofectamine 2000 according to the manufacturer's directions with DNA encoding a prime editor, PEgRNA, and (if applicable) ngRNA. Alternatively, cells are transfected with MessengerMax according to the manufacturer's directions with mRNA encoding a prime editor, synthetic PEgRNA, and (if applicable) ngRNA. Three days after transfection, gDNA is harvested in lysis buffer for high throughput sequencing and sequenced using miseq.

Lentiviral production and cell line generation: Lentiviral transfer plasmids containing the H1069Q mutation with flanking sequences from the ATP7B gene on each side, and an IRES-Puromycin selection cassette, are cloned behind an EF1α short promoter. HEK 293T cells are transiently transfected with the transfer plasmids and packaging plasmids containing VSV glycoprotein and lentiviral gag/pol coding sequences. After transfection, lentiviral particles are harvested from the cell media and concentrated. HEK 293T cells are transduced using serial dilutions of the lentiviral particles described above. Cells generated at a dilution of MOI<1, as determined by survival following puromycin, are selected for expansion. A resulting HEK293T cell line carrying the H1069Q mutation is used to screen PEgRNAs.

ATP7B H1069Q mutation installation: An ATP7B H1069Q mutation is installed at the endogenous ATP7B locus in HEK 293T, Huh-7, and HepG2 cells by prime editing and single-cell clones are obtained via limiting dilution and clonal expansion.

Prime Editing in primary human hepatocytes: Primary human hepatocytes are transduced with lentivirus encoding the H1069Q cassette 2 days after cryorecovery, followed 6 days later by transfection with RNA encoding a prime editor, PEgRNA, and (if applicable) ngRNA. Genomic DNA is harvested after a 1-week incubation.

### Example 2—Screen of Cas9 Cutting Activity at Spacers within 200 nt of the ATP7B H1069Q Mutation Site

A spacer screen was performed to investigate Cas9 cutting activity at sites within 200 nucleotides (nts) of the H1069Q mutation site in the ATP7B gene. HEK293T cells were cultured and transfected with mRNA encoding a Cas9 and synthetic sgRNA as described above. The results are shown in Table XA.

TABLE XA
Spacer screen for Cas9 cutting activity within 200
nt of the H1069Q mutation site in the ATP7B gene

| | | | | PAM Location |
|---|---|---|---|---|
| | Nick-to-edit | | | Relative to |
| Spacer¹ | distance to H1069 | | Percent | H1069Q Mutation |
| SEQ ID NO: | (nt)² | PAM | Indel³ | site⁴ |
| 5281 | −85 | NGA | +++ | 5′ |
| 738 | −78 | NGG | +++ | 5′ |
| 740 | −75 | NGG | ++ | 5′ |
| 736 | −74 | NGG | ++ | 5′ |
| 737 | −73 | NGG | ++ | 5′ |
| 529 | −72 | NGA | ++ | 5′ |
| 200 | −70 | NGG | + | 5′ |
| 199 | −69 | NGG | ++ | 5′ |
| 35 | −65 | NGG | + | 5′ |
| 55 | −64 | NGG | + | 5′ |
| 2090 | −51 | NGG | + | 3′ |
| 2063 | −46 | NGG | ++ | 3′ |
| 2048 | −45 | NGG | + | 3′ |
| 2066 | −40 | NGA | + | 3′ |
| 2051 | −39 | NGG | +++ | 3′ |
| 2045 | −32 | NGG | ++++ | 3′ |
| 2091 | −30 | NGA | +++ | 3′ |
| 45; 2023 | −27 | NGG | ++ | 5′ |
| 43 | −24 | NGA | + | 5′ |
| 33; 2293 | −9 | NGG | ++ | 5′ |
| 58; 4425 | −8 | NGG | +++ | 5′ |
| 53 | −7 | NGG | ++ | 5′ |
| 2076; 5248 | 9 | NGA | ++ | 5′ |
| 1505; 2055 | 19 | NGG | +++ | 5′ |
| 2081; 5228 | 21 | NGA | + | 5′ |
| 682; 2087 | 22 | NGG | + | 5′ |
| 2083; 5206 | 28 | NGA | + | 5′ |
| 483; 2061 | 29 | NGG | ++++ | 5′ |
| 294; 2070 | 30 | NGG | ++++ | 5′ |
| 182; 2080 | 31 | NGG | +++ | 5′ |
| 42 | 32 | NGA | + | 3′ |
| 1; 2058 | 34 | NGG | +++ | 5′ |
| 2069; 5555 | 40 | NGG | ++++ | 5′ |
| 39 | 42 | NGA | ++++ | 3′ |
| 70 | 44 | NGG | ++++ | 3′ |
| 68 | 45 | NGG | +++ | 3′ |
| 47 | 46 | NGG | ++++ | 3′ |
| 2057; 5706 | 46 | NGG | ++++ | 5′ |
| 2064 | 49 | NGA | + | 5′ |
| 2056; 5638 | 50 | NGG | +++ | 5′ |
| 32 | 54 | NGG | +++ | 3′ |
| 2068 | 67 | NGG | + | 5′ |
| 31 | 71 | NGG | +++ | 3′ |
| 40 | 72 | NGA | ++++ | 3′ |
| 2046 | 72 | NGA | + | 5′ |
| 2059 | 73 | NGG | ++ | 5′ |
| 2089 | 74 | NGG | +++ | 5′ |
| 2443 | 75 | NGG | ++ | 5′ |
| 2444 | 76 | NGG | +++ | 5′ |
| 20 | 84 | NGA | + | 3′ |
| 2437 | 88 | NGG | ++++ | 5′ |
| 2439 | 89 | NGG | ++++ | 5′ |
| 30 | 90 | NGG | +++ | 3′ |
| 27 | 91 | NGG | +++ | 3′ |
| 36 | 92 | NGG | +++ | 3′ |
| 21 | 93 | NGA | + | 3′ |
| 5536 | 97 | NGG | ++++ | 5′ |
| 69 | 98 | NGG | ++++ | 5′ |
| 50 | 101 | NGG | +++ | 3′ |
| 34 | 102 | NGG | ++ | 3′ |
| 62 | 108 | NGG | +++ | 5′ |
| 72 | 108 | NGG | ++ | 5′ |
| 65 | 123 | NGG | ++++ | 5′ |
| 5219 | 127 | NGA | + | 5′ |
| 5220 | 132 | NGA | + | 5′ |
| 5226 | 139 | NGA | ++ | 5′ |
| 5221 | 145 | NGA | + | 5′ |
| 5222 | 151 | NGA | + | 5′ |
| 41 | 155 | NGG | ++ | 5′ |
| 63 | 165 | NGG | ++++ | 5′ |
| 5223 | 166 | NGA | ++ | 3′ |
| 5227 | 172 | NGA | ++++ | 5′ |
| 5224 | 178 | NGA | ++ | 3′ |
| 64 | 179 | NGG | ++++ | 5′ |
| 66 | 181 | NGG | ++ | 3′ |
| 5225 | 182 | NGA | +++ | 3′ |
| 61 | 184 | NGG | ++++ | 3′ |
| 71 | 185 | NGG | ++++ | 3′ |
| 60 | 186 | NGG | ++++ | 3′ |

¹The indicated sequence sequences recite only the spacer; the sgRNA used experimentally contained the gRNA core of SEQ ID NO: 5957, a 3′ mU*mU*mU*U modification, and a 5′mN*mN*mN* modification, where m indicates that the indicated nucleotide contains a 2′-O— Me modification and a * indicates a phosphorothioate bond. Some spacers are identified by two SEQ ID NOs because the same spacer sequence was assigned a different SEQ ID NO in the cluster tables depending upon whether it was included as a ngRNA spacer or a PEgRNA spacer.
²A (+) nick-to-edit distance indicates the PAM is on the sense strand whereas a (−) nick-to-edit distance indicates the PAM is on the antisense strand.
³+ = 0.0%-3.3%; ++ = 3.3%-7.2%; +++ 7.2%-20%; ++++ = 20%-52.9%
⁴The indication of 5′ or 3′ refers to the position of the PAM relative to the H1069Q mutation site on the PAM strand in the ATP7B gene. The H1069Q mutation site may therefore refer to the sense or antisense strand, depending upon which strand contains the PAM sequence.

### Example 3—Prime Editing at a Lentivirus-Introduced ATP7B H1069Q Mutation Site in HEK293T Cells Using a PE2 System

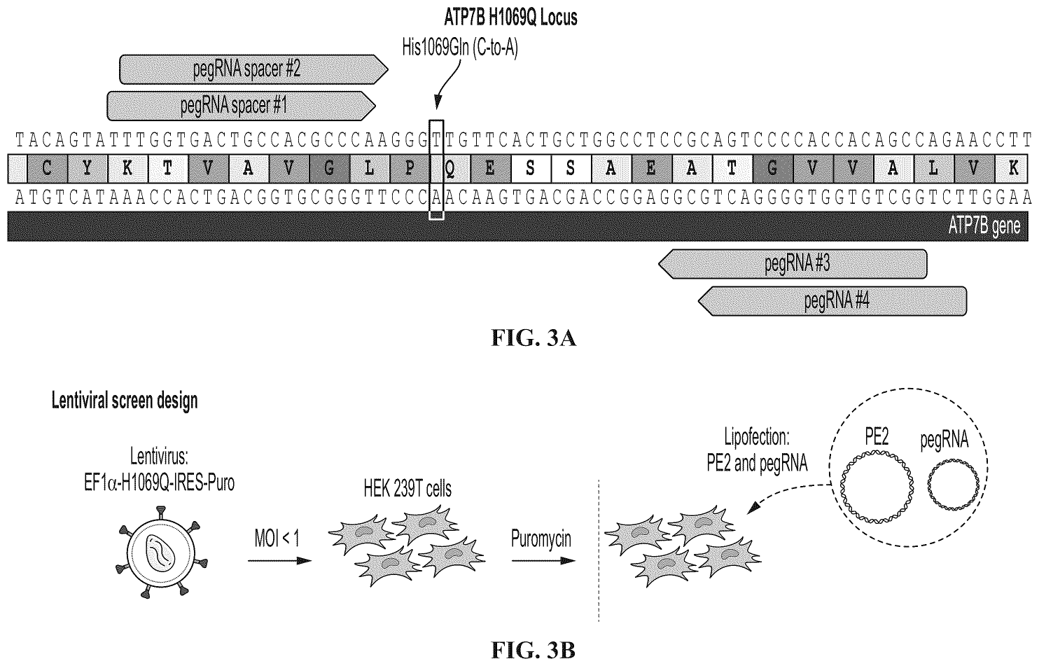

Four exemplary PEgRNA spacers close to the H1069Q mutation are shown in FIG. 3A. In FIG. 3A, pegRNA spacer #1 corresponds to SEQ ID NO: 2293, pegRNA spacer #2 corresponds to SEQ ID NO: 4452, pegRNA spacer #3 corresponds to SEQ ID NO: 1505, and pegRNA spacer #4 corresponds to SEQ ID NO: 682. PEgRNA incorporating these spacers were designed and screened for Prime Editing efficiency in a HEK293T cell line containing a lentivirus-introduced H1069Q mutation. The cell line was generated as described in Example 1. These spacers were selected because they are close to the H1069Q mutation site and would produce a nick that is 5′ of the H1069Q mutation site when used in conjunction with a prime editor having a Cas9 protein containing an inactivating mutation in the HNH nuclease domain. Each of these spacers also showed at least some activity in the spacer screen of Example 2.

375 PEgRNA were designed and screened in a PE2 system (i.e., without a ngRNA). In this initial screen, multiple primer binding site (PBS) and reverse transcription template (RTT) lengths were tested for each of the four exemplary spacers. All the PEgRNA were designed to restore the wild-type nucleic acid sequence at the H1069Q site. Where possible, additional PEgRNAs were designed that also introduce a silent mutation that destroys the PAM sequence (i.e., a PAM silencing mutation).

The results for individual PEgRNA are shown in Table XB. Successful Prime Editing was observed across PBS and RTT lengths, with and without PAM silencing. The percent editing observed for all PEgRNA having the same spacer were averaged, and the results reported in Table XC. The relative levels of Prime Editing observed between spacers is similar to the relative levels of Cas9 cutting for these spacers in the spacer screen of Example 2.

TABLE XB
PE2 Screen at H1069Q mutation site in HEK293T cells

| PEgRNA¹ | Spacer | RTT² | | PBS | | |
|---|---|---|---|---|---|---|
| SEQ ID | SEQ | SEQ | RTT | SEQ | PBS | Percent |
| NO: | ID NO: | ID NO: | Length | ID NO: | Length | Edit³ |
| 1565 | 1505; 2055 | 1518 | 21 | 1509 | 10 | +++ |
| 1564 | 1505; 2055 | 1517* | 21 | 1509 | 10 | + |
| 1569 | 1505; 2055 | 1519 | 22 | 1509 | 10 | ++++ |
| 1575 | 1505; 2055 | 1520* | 22 | 1509 | 10 | + |
| 1580 | 1505; 2055 | 1522 | 23 | 1509 | 10 | + |
| 1577 | 1505; 2055 | 1521* | 23 | 1509 | 10 | +++ |
| 1593 | 1505; 2055 | 1524 | 24 | 1509 | 10 | ++ |
| 1590 | 1505; 2055 | 1523* | 24 | 1509 | 10 | +++ |
| 1600 | 1505; 2055 | 1526 | 25 | 1509 | 10 | +++ |
| 1598 | 1505; 2055 | 1525* | 25 | 1509 | 10 | ++++ |
| 1578 | 1505; 2055 | 1518 | 21 | 1511 | 12 | +++ |
| 1586 | 1505; 2055 | 1517* | 21 | 1511 | 12 | + |
| 1591 | 1505; 2055 | 1519 | 22 | 1511 | 12 | +++ |
| 1587 | 1505; 2055 | 1520* | 22 | 1511 | 12 | +++ |
| 1609 | 1505; 2055 | 1522 | 23 | 1511 | 12 | ++ |
| 1607 | 1505; 2055 | 1521* | 23 | 1511 | 12 | +++ |
| 1616 | 1505; 2055 | 1524 | 24 | 1511 | 12 | ++++ |
| 1622 | 1505; 2055 | 1523* | 24 | 1511 | 12 | ++++ |
| 1643 | 1505; 2055 | 1526 | 25 | 1511 | 12 | +++ |
| 1637 | 1505; 2055 | 1525* | 25 | 1511 | 12 | ++++ |
| 1602 | 1505; 2055 | 1518 | 21 | 1513 | 14 | ++ |
| 1606 | 1505; 2055 | 1517* | 21 | 1513 | 14 | + |
| 1626 | 1505; 2055 | 1519 | 22 | 1513 | 14 | +++ |
| 1615 | 1505; 2055 | 1520* | 22 | 1513 | 14 | ++++ |
| 1628 | 1505; 2055 | 1522 | 23 | 1513 | 14 | +++ |
| 1645 | 1505; 2055 | 1521* | 23 | 1513 | 14 | ++ |
| 1664 | 1505; 2055 | 1524 | 24 | 1513 | 14 | ++ |
| 1668 | 1505; 2055 | 1523* | 24 | 1513 | 14 | +++ |
| 1671 | 1505; 2055 | 1526 | 25 | 1513 | 14 | ++ |
| 1679 | 1505; 2055 | 1525* | 25 | 1513 | 14 | ++++ |
| 1640 | 1505; 2055 | 1518 | 21 | 1515 | 16 | ++ |
| 1646 | 1505; 2055 | 1517* | 21 | 1515 | 16 | + |
| 1654 | 1505; 2055 | 1519 | 22 | 1515 | 16 | + |
| 1663 | 1505; 2055 | 1520* | 22 | 1515 | 16 | ++++ |
| 1691 | 1505; 2055 | 1522 | 23 | 1515 | 16 | + |
| 1678 | 1505; 2055 | 1521* | 23 | 1515 | 16 | ++ |
| 1708 | 1505; 2055 | 1524 | 24 | 1515 | 16 | ++ |
| 1694 | 1505; 2055 | 1523* | 24 | 1515 | 16 | +++ |
| 1725 | 1505; 2055 | 1526 | 25 | 1515 | 16 | ++++ |
| 1721 | 1505; 2055 | 1525* | 25 | 1515 | 16 | ++++ |
| 1550 | 1505; 2055 | 1518 | 21 | 1507 | 8 | +++ |
| 1551 | 1505; 2055 | 1517* | 21 | 1507 | 8 | +++ |
| 1554 | 1505; 2055 | 1519 | 22 | 1507 | 8 | +++ |
| 1553 | 1505; 2055 | 1520* | 22 | 1507 | 8 | ++ |
| 1562 | 1505; 2055 | 1522 | 23 | 1507 | 8 | + |
| 1566 | 1505; 2055 | 1521* | 23 | 1507 | 8 | +++ |
| 1570 | 1505; 2055 | 1524 | 24 | 1507 | 8 | ++ |
| 1567 | 1505; 2055 | 1523* | 24 | 1507 | 8 | ++ |
| 1583 | 1505; 2055 | 1526 | 25 | 1507 | 8 | ++ |
| 1584 | 1505; 2055 | 1525* | 25 | 1507 | 8 | ++ |
| 777 | 682; 2087 | 697 | 24 | 686 | 10 | + |
| 778 | 682; 2087 | 694* | 24 | 686 | 10 | + |
| 775 | 682; 2087 | 695* | 24 | 686 | 10 | + |
| 772 | 682; 2087 | 696* | 24 | 686 | 10 | + |
| 781 | 682; 2087 | 699 | 25 | 686 | 10 | + |
| 787 | 682; 2087 | 701* | 25 | 686 | 10 | + |
| 784 | 682; 2087 | 698* | 25 | 686 | 10 | + |
| 791 | 682; 2087 | 700* | 25 | 686 | 10 | + |
| 807 | 682; 2087 | 702 | 26 | 686 | 10 | + |
| 820 | 682; 2087 | 704* | 26 | 686 | 10 | + |
| 801 | 682; 2087 | 703* | 26 | 686 | 10 | + |
| 813 | 682; 2087 | 705* | 26 | 686 | 10 | + |
| 838 | 682; 2087 | 707 | 27 | 686 | 10 | ++ |
| 828 | 682; 2087 | 708* | 27 | 686 | 10 | +++ |
| 829 | 682; 2087 | 709* | 27 | 686 | 10 | ++ |
| 824 | 682; 2087 | 706* | 27 | 686 | 10 | ++ |
| 852 | 682; 2087 | 711 | 28 | 686 | 10 | +++ |
| 854 | 682; 2087 | 712* | 28 | 686 | 10 | ++ |
| 860 | 682; 2087 | 710* | 28 | 686 | 10 | +++ |
| 856 | 682; 2087 | 713* | 28 | 686 | 10 | ++ |
| 819 | 682; 2087 | 697 | 24 | 688 | 12 | + |
| 811 | 682; 2087 | 694* | 24 | 688 | 12 | + |
| 815 | 682; 2087 | 695* | 24 | 688 | 12 | + |
| 805 | 682; 2087 | 696* | 24 | 688 | 12 | + |
| 825 | 682; 2087 | 699 | 25 | 688 | 12 | + |
| 822 | 682; 2087 | 701* | 25 | 688 | 12 | + |
| 832 | 682; 2087 | 698* | 25 | 688 | 12 | + |
| 835 | 682; 2087 | 700* | 25 | 688 | 12 | + |
| 843 | 682; 2087 | 702 | 26 | 688 | 12 | + |
| 849 | 682; 2087 | 704* | 26 | 688 | 12 | + |
| 853 | 682; 2087 | 703* | 26 | 688 | 12 | + |
| 858 | 682; 2087 | 705* | 26 | 688 | 12 | +++ |
| 882 | 682; 2087 | 707 | 27 | 688 | 12 | ++ |
| 880 | 682; 2087 | 708* | 27 | 688 | 12 | ++ |
| 870 | 682; 2087 | 709* | 27 | 688 | 12 | ++ |
| 879 | 682; 2087 | 706* | 27 | 688 | 12 | ++ |
| 896 | 682; 2087 | 711 | 28 | 688 | 12 | + |
| 920 | 682; 2087 | 712* | 28 | 688 | 12 | +++ |
| 900 | 682; 2087 | 710* | 28 | 688 | 12 | ++ |
| 908 | 682; 2087 | 713* | 28 | 688 | 12 | ++ |
| 851 | 682; 2087 | 697 | 24 | 690 | 14 | + |
| 857 | 682; 2087 | 694* | 24 | 690 | 14 | + |
| 841 | 682; 2087 | 695* | 24 | 690 | 14 | + |
| 846 | 682; 2087 | 696* | 24 | 690 | 14 | + |
| 861 | 682; 2087 | 699 | 25 | 690 | 14 | ++ |
| 873 | 682; 2087 | 701* | 25 | 690 | 14 | + |
| 878 | 682; 2087 | 698* | 25 | 690 | 14 | + |
| 887 | 682; 2087 | 700* | 25 | 690 | 14 | + |
| 906 | 682; 2087 | 702 | 26 | 690 | 14 | + |
| 912 | 682; 2087 | 704* | 26 | 690 | 14 | + |
| 893 | 682; 2087 | 703* | 26 | 690 | 14 | + |
| 916 | 682; 2087 | 705* | 26 | 690 | 14 | + |
| 929 | 682; 2087 | 707 | 27 | 690 | 14 | + |
| 960 | 682; 2087 | 708* | 27 | 690 | 14 | ++ |
| 923 | 682; 2087 | 709* | 27 | 690 | 14 | ++ |
| 934 | 682; 2087 | 706* | 27 | 690 | 14 | + |
| 989 | 682; 2087 | 711 | 28 | 690 | 14 | +++ |
| 961 | 682; 2087 | 712* | 28 | 690 | 14 | +++ |
| 997 | 682; 2087 | 710* | 28 | 690 | 14 | ++ |
| 971 | 682; 2087 | 713* | 28 | 690 | 14 | ++ |
| 914 | 682; 2087 | 697 | 24 | 692 | 16 | + |
| 905 | 682; 2087 | 694* | 24 | 692 | 16 | + |
| 902 | 682; 2087 | 695* | 24 | 692 | 16 | + |
| 898 | 682; 2087 | 696* | 24 | 692 | 16 | + |
| 926 | 682; 2087 | 699 | 25 | 692 | 16 | + |
| 927 | 682; 2087 | 701* | 25 | 692 | 16 | 1 |
| 938 | 682; 2087 | 698* | 25 | 692 | 16 | + |
| 935 | 682; 2087 | 700* | 25 | 692 | 16 | + |
| 977 | 682; 2087 | 702 | 26 | 692 | 16 | ++ |
| 983 | 682; 2087 | 704* | 26 | 692 | 16 | + |
| 963 | 682; 2087 | 703* | 26 | 692 | 16 | + |
| 970 | 682; 2087 | 705* | 26 | 692 | 16 | + |
| 1041 | 682; 2087 | 707 | 27 | 692 | 16 | + |
| 1020 | 682; 2087 | 708* | 27 | 692 | 16 | ++ |
| 1043 | 682; 2087 | 709* | 27 | 692 | 16 | + |
| 1024 | 682; 2087 | 706* | 27 | 692 | 16 | + |
| 1074 | 682; 2087 | 711 | 28 | 692 | 16 | ++ |
| 1057 | 682; 2087 | 712* | 28 | 692 | 16 | ++ |
| 1068 | 682; 2087 | 710* | 28 | 692 | 16 | ++ |
| 1085 | 682; 2087 | 713* | 28 | 692 | 16 | +++ |
| 752 | 682; 2087 | 697 | 24 | 684 | 8 | + |
| 745 | 682; 2087 | 694* | 24 | 684 | 8 | + |
| 751 | 682; 2087 | 695* | 24 | 684 | 8 | + |
| 746 | 682; 2087 | 696 | 24 | 684 | 8 | + |
| 760 | 682; 2087 | 699 | 25 | 684 | 8 | + |
| 758 | 682; 2087 | 701* | 25 | 684 | 8 | + |
| 756 | 682; 2087 | 698* | 25 | 684 | 8 | + |
| 761 | 682; 2087 | 700* | 25 | 684 | 8 | + |
| 765 | 682; 2087 | 702 | 26 | 684 | 8 | + |
| 767 | 682; 2087 | 704* | 26 | 684 | 8 | + |
| 770 | 682; 2087 | 703* | 26 | 684 | 8 | + |
| 780 | 682; 2087 | 705* | 26 | 684 | 8 | + |
| 783 | 682; 2087 | 707 | 27 | 684 | 8 | + |
| 796 | 682; 2087 | 708* | 27 | 684 | 8 | ++ |
| 790 | 682; 2087 | 709* | 27 | 684 | 8 | + |
| 789 | 682; 2087 | 706* | 27 | 684 | 8 | + |
| 804 | 682; 2087 | 711 | 28 | 684 | 8 | ++ |
| 812 | 682; 2087 | 712* | 28 | 684 | 8 | +++ |
| 809 | 682; 2087 | 710* | 28 | 684 | 8 | + |
| 816 | 682; 2087 | 713* | 28 | 684 | 8 | ++ |
| 4507 | 58; 4425 | 4438 | 10 | 4429 | 10 | +++ |
| 4527 | 58; 4425 | 4441 | 12 | 4429 | 10 | ++++ |
| 4533 | 58; 4425 | 4444 | 13 | 4429 | 10 | ++++ |
| 4555 | 58; 4425 | 4446 | 14 | 4429 | 10 | ++ |
| 4564 | 58; 4425 | 4448 | 15 | 4429 | 10 | + |
| 4649 | 58; 4425 | 4452 | 18 | 4429 | 10 | ++++ |
| 4756 | 58; 4425 | 4456 | 21 | 4429 | 10 | ++++ |
| 4519 | 58; 4425 | 4438 | 10 | 4431 | 12 | ++++ |
| 4542 | 58; 4425 | 4440 | 11 | 4431 | 12 | ++++ |
| 4559 | 58; 4425 | 4441 | 12 | 4431 | 12 | ++ |
| 4576 | 58; 4425 | 4444 | 13 | 4431 | 12 | ++++ |
| 4588 | 58; 4425 | 4446 | 14 | 4431 | 12 | + |
| 4624 | 58; 4425 | 4448 | 15 | 4431 | 12 | + |
| 4679 | 58; 4425 | 4450 | 17 | 4431 | 12 | ++++ |
| 4697 | 58; 4425 | 4452 | 18 | 4431 | 12 | ++ |
| 4817 | 58; 4425 | 4456 | 21 | 4431 | 12 | ++++ |
| 4553 | 58; 4425 | 4438 | 10 | 4433 | 14 | +++ |
| 4562 | 58; 4425 | 4440 | 11 | 4433 | 14 | ++ |
| 4592 | 58; 4425 | 4441 | 12 | 4433 | 14 | ++++ |
| 4640 | 58; 4425 | 4446 | 14 | 4433 | 14 | ++++ |
| 4691 | 58; 4425 | 4448 | 15 | 4433 | 14 | ++++ |
| 4726 | 58; 4425 | 4450 | 17 | 4433 | 14 | ++++ |
| 4761 | 58; 4425 | 4452 | 18 | 4433 | 14 | ++++ |
| 4852 | 58; 4425 | 4456 | 21 | 4433 | 14 | ++++ |
| 4629 | 58; 4425 | 4440 | 11 | 4435 | 16 | ++ |
| 4653 | 58; 4425 | 4441 | 12 | 4435 | 16 | ++++ |
| 4668 | 58; 4425 | 4444 | 13 | 4435 | 16 | ++++ |
| 4724 | 58; 4425 | 4446 | 14 | 4435 | 16 | ++ |
| 4738 | 58; 4425 | 4448 | 15 | 4435 | 16 | + |
| 4809 | 58; 4425 | 4450 | 17 | 4435 | 16 | +++ |
| 4840 | 58; 4425 | 4452 | 18 | 4435 | 16 | +++ |
| 4893 | 58; 4425 | 4456 | 21 | 4435 | 16 | ++++ |
| 4500 | 58; 4425 | 4440 | 11 | 4427 | 8 | ++++ |
| 4504 | 58; 4425 | 4441 | 12 | 4427 | 8 | + |
| 4517 | 58; 4425 | 4444 | 13 | 4427 | 8 | +++ |
| 4522 | 58; 4425 | 4446 | 14 | 4427 | 8 | ++++ |
| 4540 | 58; 4425 | 4448 | 15 | 4427 | 8 | ++++ |
| 4580 | 58; 4425 | 4450 | 17 | 4427 | 8 | ++++ |
| 4591 | 58; 4425 | 4452 | 18 | 4427 | 8 | ++++ |
| 2480 | 33; 2293 | 2307 | 11 | 2297 | 10 | +++ |
| 2478 | 33; 2293 | 2308* | 11 | 2297 | 10 | + |
| 2487 | 33; 2293 | 2310* | 11 | 2297 | 10 | ++ |
| 2488 | 33; 2293 | 2306* | 11 | 2297 | 10 | ++ |
| 2510 | 33; 2293 | 2313 | 12 | 2297 | 10 | ++++ |
| 2514 | 33; 2293 | 2312* | 12 | 2297 | 10 | + |
| 2513 | 33; 2293 | 2311* | 12 | 2297 | 10 | ++ |
| 2493 | 33; 2293 | 2316* | 12 | 2297 | 10 | ++ |
| 2533 | 33; 2293 | 2320 | 13 | 2297 | 10 | ++++ |
| 2520 | 33; 2293 | 2318* | 13 | 2297 | 10 | ++ |
| 2531 | 33; 2293 | 2321* | 13 | 2297 | 10 | + |
| 2527 | 33; 2293 | 2319* | 13 | 2297 | 10 | +++ |
| 2559 | 33; 2293 | 2326 | 14 | 2297 | 10 | +++ |
| 2553 | 33; 2293 | 2324* | 14 | 2297 | 10 | ++ |
| 2561 | 33; 2293 | 2328* | 14 | 2297 | 10 | +++ |
| 2571 | 33; 2293 | 2325* | 14 | 2297 | 10 | ++++ |
| 2605 | 33; 2293 | 2329 | 15 | 2297 | 10 | +++ |
| 2618 | 33; 2293 | 2333* | 15 | 2297 | 10 | |
| 2615 | 33; 2293 | 2334* | 15 | 2297 | 10 | ++++ |
| 2598 | 33; 2293 | 2332* | 15 | 2297 | 10 | +++ |
| 2633 | 33; 2293 | 2335 | 16 | 2297 | 10 | ++++ |
| 2632 | 33; 2293 | 2338* | 16 | 2297 | 10 | +++ |
| 2645 | 33; 2293 | 2339* | 16 | 2297 | 10 | ++++ |
| 2634 | 33; 2293 | 2337* | 16 | 2297 | 10 | +++ |
| 2769 | 33; 2293 | 2342 | 18 | 2297 | 10 | ++++ |
| 2773 | 33; 2293 | 2346* | 18 | 2297 | 10 | ++ |
| 2772 | 33; 2293 | 2343* | 18 | 2297 | 10 | +++ |
| 2765 | 33; 2293 | 2344* | 18 | 2297 | 10 | +++ |
| 2801 | 33; 2293 | 2351 | 19 | 2297 | 10 | ++++ |
| 2829 | 33; 2293 | 2347* | 19 | 2297 | 10 | ++ |
| 2826 | 33; 2293 | 2348* | 19 | 2297 | 10 | +++ |
| 2807 | 33; 2293 | 2352* | 19 | 2297 | 10 | +++ |
| 2983 | 33; 2293 | 2353 | 22 | 2297 | 10 | +++ |
| 3023 | 33; 2293 | 2356* | 22 | 2297 | 10 | ++ |
| 3012 | 33; 2293 | 2355* | 22 | 2297 | 10 | +++ |
| 2977 | 33; 2293 | 2358* | 22 | 2297 | 10 | +++ |
| 2522 | 33; 2293 | 2307 | 11 | 2299 | 12 | +++ |
| 2528 | 33; 2293 | 2308* | 11 | 2299 | 12 | ++ |
| 2526 | 33; 2293 | 2310* | 11 | 2299 | 12 | ++ |
| 2544 | 33; 2293 | 2306* | 11 | 2299 | 12 | ++++ |
| 2575 | 33; 2293 | 2313 | 12 | 2299 | 12 | ++++ |
| 2562 | 33; 2293 | 2312* | 12 | 2299 | 12 | ++ |
| 2554 | 33; 2293 | 2311* | 12 | 2299 | 12 | ++ |
| 2563 | 33; 2293 | 2316* | 12 | 2299 | 12 | ++ |
| 2585 | 33; 2293 | 2320 | 13 | 2299 | 12 | ++++ |
| 2596 | 33; 2293 | 2318* | 13 | 2299 | 12 | + |
| 2610 | 33; 2293 | 2321* | 13 | 2299 | 12 | ++ |
| 2590 | 33; 2293 | 2319* | 13 | 2299 | 12 | +++ |
| 2628 | 33; 2293 | 2326 | 14 | 2299 | 12 | ++++ |
| 2646 | 33; 2293 | 2324* | 14 | 2299 | 12 | + |
| 2643 | 33; 2293 | 2328* | 14 | 2299 | 12 | +++ |
| 2665 | 33; 2293 | 2325* | 14 | 2299 | 12 | +++ |
| 2667 | 33; 2293 | 2329 | 15 | 2299 | 12 | ++++ |
| 2699 | 33; 2293 | 2333* | 15 | 2299 | 12 | ++ |
| 2701 | 33; 2293 | 2334* | 15 | 2299 | 12 | +++ |
| 2674 | 33; 2293 | 2332* | 15 | 2299 | 12 | +++ |
| 2744 | 33; 2293 | 2335 | 16 | 2299 | 12 | ++++ |
| 2734 | 33; 2293 | 2338* | 16 | 2299 | 12 | ++ |
| 2735 | 33; 2293 | 2339* | 16 | 2299 | 12 | +++ |
| 2733 | 33; 2293 | 2337* | 16 | 2299 | 12 | ++++ |
| 2886 | 33; 2293 | 2342 | 18 | 2299 | 12 | ++++ |
| 2899 | 33; 2293 | 2346* | 18 | 2299 | 12 | +++ |
| 2885 | 33; 2293 | 2343* | 18 | 2299 | 12 | +++ |
| 2890 | 33; 2293 | 2344* | 18 | 2299 | 12 | ++++ |
| 2916 | 33; 2293 | 2351 | 19 | 2299 | 12 | ++++ |
| 2941 | 33; 2293 | 2347* | 19 | 2299 | 12 | ++ |
| 2960 | 33; 2293 | 2348* | 19 | 2299 | 12 | +++ |
| 2962 | 33; 2293 | 2352* | 19 | 2299 | 12 | +++ |
| 3163 | 33; 2293 | 2353 | 22 | 2299 | 12 | +++ |
| 3122 | 33; 2293 | 2356* | 22 | 2299 | 12 | +++ |
| 3188 | 33; 2293 | 2355* | 22 | 2299 | 12 | +++ |
| 3166 | 33; 2293 | 2358* | 22 | 2299 | 12 | +++ |
| 2606 | 33; 2293 | 2307 | 11 | 2301 | 14 | ++ |
| 2602 | 33; 2293 | 2308* | 11 | 2301 | 14 | + |
| 2609 | 33; 2293 | 2310* | 11 | 2301 | 14 | ++ |
| 2584 | 33; 2293 | 2306* | 11 | 2301 | 14 | ++ |
| 2623 | 33; 2293 | 2313 | 12 | 2301 | 14 | ++++ |
| 2647 | 33; 2293 | 2312* | 12 | 2301 | 14 | ++ |
| 2660 | 33; 2293 | 2311* | 12 | 2301 | 14 | ++ |
| 2619 | 33; 2293 | 2316* | 12 | 2301 | 14 | +++ |
| 2675 | 33; 2293 | 2320 | 13 | 2301 | 14 | ++++ |
| 2677 | 33; 2293 | 2318* | 13 | 2301 | 14 | ++ |
| 2685 | 33; 2293 | 2321* | 13 | 2301 | 14 | ++ |
| 2670 | 33; 2293 | 2319* | 13 | 2301 | 14 | +++ |
| 2729 | 33; 2293 | 2326 | 14 | 2301 | 14 | ++++ |
| 2749 | 33; 2293 | 2324* | 14 | 2301 | 14 | ++ |
| 2764 | 33; 2293 | 2328* | 14 | 2301 | 14 | +++ |
| 2730 | 33; 2293 | 2325* | 14 | 2301 | 14 | +++ |
| 2802 | 33; 2293 | 2329 | 15 | 2301 | 14 | ++++ |
| 2816 | 33; 2293 | 2333* | 15 | 2301 | 14 | +++ |
| 2777 | 33; 2293 | 2334* | 15 | 2301 | 14 | ++++ |
| 2832 | 33; 2293 | 2332* | 15 | 2301 | 14 | ++++ |
| 2898 | 33; 2293 | 2335 | 16 | 2301 | 14 | ++++ |
| 2834 | 33; 2293 | 2338* | 16 | 2301 | 14 | +++ |
| 2893 | 33; 2293 | 2339* | 16 | 2301 | 14 | ++++ |
| 2889 | 33; 2293 | 2337* | 16 | 2301 | 14 | ++++ |
| 3043 | 33; 2293 | 2342 | 18 | 2301 | 14 | ++++ |
| 3013 | 33; 2293 | 2346* | 18 | 2301 | 14 | ++ |
| 3015 | 33; 2293 | 2343* | 18 | 2301 | 14 | ++++ |
| 2982 | 33; 2293 | 2344* | 18 | 2301 | 14 | ++++ |
| 3076 | 33; 2293 | 2351 | 19 | 2301 | 14 | ++++ |
| 3071 | 33; 2293 | 2347* | 19 | 2301 | 14 | +++ |
| 3080 | 33; 2293 | 2348* | 19 | 2301 | 14 | ++++ |
| 3072 | 33; 2293 | 2352* | 19 | 2301 | 14 | ++++ |
| 3287 | 33; 2293 | 2353 | 22 | 2301 | 14 | +++ |
| 3275 | 33; 2293 | 2356* | 22 | 2301 | 14 | ++ |
| 3301 | 33; 2293 | 2355* | 22 | 2301 | 14 | +++ |
| 3300 | 33; 2293 | 2358* | 22 | 2301 | 14 | +++ |
| 2683 | 33; 2293 | 2307 | 11 | 2303 | 16 | ++++ |
| 2711 | 33; 2293 | 2308* | 11 | 2303 | 16 | + |
| 2680 | 33; 2293 | 2310* | 11 | 2303 | 16 | ++ |
| 2702 | 33; 2293 | 2306* | 11 | 2303 | 16 | ++++ |
| 2758 | 33; 2293 | 2313 | 12 | 2303 | 16 | ++++ |
| 2760 | 33; 2293 | 2312* | 12 | 2303 | 16 | ++ |
| 2759 | 33; 2293 | 2311* | 12 | 2303 | 16 | ++ |
| 2757 | 33; 2293 | 2316* | 12 | 2303 | 16 | ++ |
| 2825 | 33; 2293 | 2320 | 13 | 2303 | 16 | +++ |
| 2830 | 33; 2293 | 2318* | 13 | 2303 | 16 | ++ |
| 2814 | 33; 2293 | 2321* | 13 | 2303 | 16 | ++ |
| 2800 | 33; 2293 | 2319* | 13 | 2303 | 16 | + |
| 2844 | 33; 2293 | 2326 | 14 | 2303 | 16 | ++++ |
| 2842 | 33; 2293 | 2324* | 14 | 2303 | 16 | ++++ |
| 2843 | 33; 2293 | 2328* | 14 | 2303 | 16 | +++ |
| 2841 | 33; 2293 | 2325* | 14 | 2303 | 16 | +++ |
| 2932 | 33; 2293 | 2329 | 15 | 2303 | 16 | ++++ |
| 2926 | 33; 2293 | 2333* | 15 | 2303 | 16 | ++++ |
| 2964 | 33; 2293 | 2334* | 15 | 2303 | 16 | +++ |
| 2933 | 33; 2293 | 2332* | 15 | 2303 | 16 | +++ |
| 2993 | 33; 2293 | 2335 | 16 | 2303 | 16 | ++++ |
| 2972 | 33; 2293 | 2338* | 16 | 2303 | 16 | ++ |
| 2988 | 33; 2293 | 2339* | 16 | 2303 | 16 | ++++ |
| 2979 | 33; 2293 | 2337* | 16 | 2303 | 16 | +++ |
| 3144 | 33; 2293 | 2342 | 18 | 2303 | 16 | +++ |
| 3148 | 33; 2293 | 2346* | 18 | 2303 | 16 | ++ |
| 3123 | 33; 2293 | 2343* | 18 | 2303 | 16 | ++++ |
| 3165 | 33; 2293 | 2344* | 18 | 2303 | 16 | ++++ |
| 3263 | 33; 2293 | 2351 | 19 | 2303 | 16 | ++++ |
| 3200 | 33; 2293 | 2347* | 19 | 2303 | 16 | ++ |
| 3248 | 33; 2293 | 2348* | 19 | 2303 | 16 | + |
| 3210 | 33; 2293 | 2352* | 19 | 2303 | 16 | +++ |
| 3441 | 33; 2293 | 2353 | 22 | 2303 | 16 | ++++ |
| 3420 | 33; 2293 | 2356* | 22 | 2303 | 16 | ++ |
| 3430 | 33; 2293 | 2355* | 22 | 2303 | 16 | +++ |
| 3442 | 33; 2293 | 2358* | 22 | 2303 | 16 | +++ |
| 2453 | 33; 2293 | 2307 | 11 | 2295 | 8 | ++ |
| 2451 | 33; 2293 | 2308* | 11 | 2295 | 8 | + |
| 2458 | 33; 2293 | 2310* | 11 | 2295 | 8 | +++ |
| 2457 | 33; 2293 | 2306* | 11 | 2295 | 8 | ++++ |
| 2459 | 33; 2293 | 2313 | 12 | 2295 | 8 | ++++ |
| 2460 | 33; 2293 | 2312* | 12 | 2295 | 8 | ++++ |
| 2466 | 33; 2293 | 2311* | 12 | 2295 | 8 | ++ |
| 2461 | 33; 2293 | 2316* | 12 | 2295 | 8 | ++ |
| 2489 | 33; 2293 | 2320 | 13 | 2295 | 8 | ++++ |
| 2473 | 33; 2293 | 2318* | 13 | 2295 | 8 | ++ |
| 2490 | 33; 2293 | 2321* | 13 | 2295 | 8 | ++++ |
| 2484 | 33; 2293 | 2319* | 13 | 2295 | 8 | +++ |
| 2515 | 33; 2293 | 2326 | 14 | 2295 | 8 | ++++ |
| 2501 | 33; 2293 | 2324* | 14 | 2295 | 8 | ++ |
| 2512 | 33; 2293 | 2328* | 14 | 2295 | 8 | +++ |
| 2509 | 33; 2293 | 2325* | 14 | 2295 | 8 | +++ |
| 2548 | 33; 2293 | 2329 | 15 | 2295 | 8 | ++++ |
| 2535 | 33; 2293 | 2333* | 15 | 2295 | 8 | ++ |
| 2546 | 33; 2293 | 2334* | 15 | 2295 | 8 | +++ |
| 2541 | 33; 2293 | 2332* | 15 | 2295 | 8 | +++ |
| 2557 | 33; 2293 | 2335 | 16 | 2295 | 8 | ++++ |
| 2564 | 33; 2293 | 2338* | 16 | 2295 | 8 | ++ |
| 2568 | 33; 2293 | 2339* | 16 | 2295 | 8 | ++++ |
| 2551 | 33; 2293 | 2337* | 16 | 2295 | 8 | +++ |
| 2637 | 33; 2293 | 2342 | 18 | 2295 | 8 | ++++ |
| 2635 | 33; 2293 | 2346* | 18 | 2295 | 8 | ++ |
| 2638 | 33; 2293 | 2343* | 18 | 2295 | 8 | +++ |
| 2636 | 33; 2293 | 2344* | 18 | 2295 | 8 | +++ |
| 2689 | 33; 2293 | 2351 | 19 | 2295 | 8 | ++++ |
| 2669 | 33; 2293 | 2347* | 19 | 2295 | 8 | ++ |
| 2690 | 33; 2293 | 2348* | 19 | 2295 | 8 | ++++ |
| 2706 | 33; 2293 | 2352* | 19 | 2295 | 8 | ++++ |
| 2871 | 33; 2293 | 2353 | 22 | 2295 | 8 | ++++ |
| 2856 | 33; 2293 | 2356* | 22 | 2295 | 8 | ++ |
| 2891 | 33; 2293 | 2355* | 22 | 2295 | 8 | +++ |
| 2894 | 33; 2293 | 2358* | 22 | 2295 | 8 | +++ |

¹Indicated PEgRNA sequence does not contain the adaptations for transcription from a DNA template used experimentally (i.e., addition of a 5'G if the spacer did not already start with a G and addition of 1-6 3'U from the U6 transcription termination sequence).
²* = RTT contains a PAM silencing mutation
³+ = 0.4%-3.3%; ++ = 3.3%-7.2%; +++ 7.2%-20%; ++++ = 20%-52.9%

TABLE XC
Average Percent Edit by Spacer in PE2 Screen
at H1069Q mutation site in HEK293T cells

| Spacer | |
|---|---|
| SEQ ID NO: | Avg. % Edit¹ |
| 1505; 2055 | +++ |
| 682; 2087 | + |
| 58; 4425 | ++++ |
| 33; 2293 | +++ |

¹+ = 0.4%-3.3%; ++ = 3.3%-7.2%; +++ 7.2%-20%; ++++ = 20%-52.9%

A subset of the PEgRNAs from Table XB were further examined for indels, the results of which are shown in Table XC. Indel frequency was quantified using standard quantification techniques via CRISPResso2 algorithm as described in Clement, K. et al., Nat. Biotechnol. 37, 224-226 (2019), with the quantification window defined as at least 20 bases 5′ and 3′ of pegRNA and ngRNA nick site.

TABLE XD
PE2 screen at the H1069 mutation site in HEK293T cells

| PEgRNA¹ | | RTT² | | PBS | | | |
|---|---|---|---|---|---|---|---|
| SEQ ID | Spacer | SEQ ID | RTT | SEQ ID | PBS | | |
| NO: | SEQ ID NO: | NO: | Length | NO: | Length | % Edit³ | % Indel³ |
| 2557 | 33; 2293 | 2335 | 16 | 2295 | 8 | ++++ | + |
| 2568 | 33; 2293 | 2339* | 16 | 2295 | 8 | ++++ | ++ |
| 2871 | 33; 2293 | 2353 | 22 | 2295 | 8 | +++ | ++ |
| 2533 | 33; 2293 | 2320 | 13 | 2297 | 10 | ++++ | ++ |
| 2615 | 33; 2293 | 2334* | 15 | 2297 | 10 | ++++ | ++ |
| 2633 | 33; 2293 | 2335 | 16 | 2297 | 10 | +++ | + |
| 2585 | 33; 2293 | 2320 | 13 | 2299 | 12 | ++++ | ++ |
| 2667 | 33; 2293 | 2329 | 15 | 2299 | 12 | ++ | ++ |
| 2744 | 33; 2293 | 2335 | 16 | 2299 | 12 | +++ | + |
| 2890 | 33; 2293 | 2344* | 18 | 2299 | 12 | +++ | + |
| 2675 | 33; 2293 | 2320 | 13 | 2301 | 14 | ++++ | ++ |
| 2729 | 33; 2293 | 2326 | 14 | 2301 | 14 | ++++ | ++ |
| 2898 | 33; 2293 | 2335 | 16 | 2301 | 14 | +++ | ++ |
| 3165 | 33; 2293 | 2344* | 18 | 2303 | 16 | ++++ | ++ |
| 2802 | 33; 2293 | 2329 | 15 | 2301 | 14 | ++++ | + |
| 4540 | 58; 4425 | 4448 | 15 | 4427 | 8 | +++ | + |
| 4533 | 58; 4425 | 4444 | 13 | 4429 | 10 | ++++ | + |
| 4756 | 58; 4425 | 4456 | 21 | 4429 | 10 | +++ | + |
| 4624 | 58; 4425 | 4448 | 15 | 4431 | 12 | ++++ | + |
| 4592 | 58; 4425 | 4441 | 12 | 4433 | 14 | ++++ | + |
| 4640 | 58; 4425 | 4446 | 14 | 4433 | 14 | ++++ | + |
| 4691 | 58; 4425 | 4448 | 15 | 4433 | 14 | ++++ | + |
| 4852 | 58; 4425 | 4456 | 21 | 4433 | 14 | +++ | + |
| 4761 | 58; 4425 | 4452 | 18 | 4433 | 14 | ++++ | + |
| 1622 | 1505; 2055 | 1523 | 24 | 1511 | 12 | +++ | ++ |
| 1615 | 1505; 2055 | 1520 | 22 | 1513 | 14 | ++++ | ++ |
| 1663 | 1505; 2055 | 1520 | 22 | 1515 | 16 | +++ | +++ |
| 1721 | 1505; 2055 | 1525 | 25 | 1515 | 16 | +++ | ++ |

¹Indicated PEgRNA sequence does not contain the adaptations for transcription from a DNA template used experimentally (i.e., addition of a 5′G if the spacer did not already start with a G and addition of 1-6 3′U from the U6 transcription termination sequence).
²* = RTT contains a PAM silencing mutation
³+ = 0.3%-3.4%; ++ = 3.4%-13.5%; +++ = 13.5%-23.0%; ++++ = 23.0%-36.0%

### Example 4—Prime Editing at the Endogenous ATP7B H1069Q Mutation Site in HEK293T Cells Using a PE3 System

An ATP7B H1069Q mutation was installed at the endogenous ATP7B locus in HEK 293T cells by prime editing and single-cell clones were obtained via limiting dilution and clonal expansion. A PE3 screen measuring correction and indel formation was performed at the endogenous ATP7B H1069Q locus. The HEK293T cells were transfected with DNA encoding a prime editor, PEgRNA, and ngRNA, as described in Example 1.

The results of the PE3 screen are provided in Tables XEa-XEd. Below each of Tables XEa-XEd is a table summarizing the PEgRNAs used experimentally. Each of the PEgRNA were tested in combination with multiple ngRNA. Some of the ngRNA were designed for a PE3B strategy and contain spacers complementary to the portion of the edit strand containing the edit. These results demonstrate the successful correction of the H1069Q mutation at the endogenous ATP7B locus in mammalian cells using both PE3 and PE3B Prime Editing systems.

TABLE XEa
PE3 screen at the H1069 mutation site in HEK293T cells

| | | PEgRNA² SEQ ID NO: | | | | | | | | | |
|---|---|---|---|---|---|---|---|---|---|---|---|
| | | 2557 | | 2568 | | 2871 | | 2533 | | 2615 | |
| | | % | % | % | % | % | % | % | % | % | % |
| | | Edit¹ | indel¹ | Edit¹ | indel¹ | Edit¹ | indel¹ | Edit¹ | indel¹ | Edit¹ | indel¹ |
| ngRNA² | PE2 | ++++ | + | ++++ | + | +++ | + | ++++ | ++ | ++++ | + |
| SEQ | 2263 | ++ | ++ | + | + | + | ++ | ++ | + | ++ | ++ |
| ID | 2266 | ++++ | ++ | + | + | ++ | ++ | +++ | + | +++ | + |
| NO: | 2276 | +++ | ++++ | ++ | ++ | + | + | + | + | ++ | +++ |
| | 2277 | ++++ | +++ | ++ | ++ | + | ++ | +++ | ++ | ++++ | ++ |
| | 2259 | ++++ | +++ | +++ | ++ | + | ++ | ++++ | ++ | ++++ | ++ |
| | 2257 | +++ | ++ | X | X | + | + | ++++ | ++ | X | X |
| | 2268 | +++ | +++ | X | X | ++ | ++ | +++ | ++ | X | X |
| | 2264 | ++++ | + | X | X | ++ | ++ | ++++ | + | X | X |
| | 2275 | ++++ | ++ | +++ | ++ | ++ | ++ | ++++ | ++ | +++ | + |
| | 2273 | ++++ | ++++ | ++ | ++ | + | + | ++++ | +++ | ++++ | ++ |
| | 2271 | ++++ | ++++ | ++ | ++ | ++ | + | +++ | +++ | +++ | +++ |
| | 2261 | ++++ | +++ | ++ | ++ | + | ++ | ++++ | +++ | ++++ | ++ |
| | 2274 | ++++ | + | +++ | + | ++ | + | ++++ | + | ++++ | + |
| | 2269 | ++++ | + | +++ | ++ | ++ | + | ++++ | + | ++++ | + |
| | 2258 | +++ | ++++ | ++ | ++ | + | ++ | +++ | +++ | +++ | +++ |
| | 2270 | ++++ | ++++ | ++ | ++ | + | ++ | +++ | +++ | +++ | ++ |
| | 2262 | ++++ | +++ | ++ | +++ | + | + | ++++ | ++ | ++++ | ++ |
| | 2267 | ++++ | + | +++ | ++ | + | + | ++++ | + | ++++ | + |
| | 2265 | ++++ | ++ | ++ | + | ++ | ++ | ++++ | + | +++ | + |
| | 2260 | ++++ | + | +++ | ++ | + | ++ | ++++ | + | ++++ | + |
| | 2272 | ++++ | ++ | +++ | + | ++ | + | +++ | + | ++++ | + |
| | 4411 | ++++ | ++ | ++ | ++ | + | + | ++++ | + | ++++ | + |
| | 4418 | ++++ | ++++ | ++ | ++ | + | + | ++++ | +++ | ++++ | ++ |
| | 4416 | ++++ | +++ | +++ | ++ | ++ | ++ | ++++ | ++ | ++++ | + |

¹+ = 0.3%-6.3%; ++ = 6.3%-12.0%; +++ = 12%-20.3%; +++ = 20.3%-55.9%; X indicates successful PE3B editing was observed with the PEgRNA (data not shown)

### Summary of PEgRNA Used:

| | | | | PBS | |
|---|---|---|---|---|---|
| PEgRNA² | Spacer | RTT³ | RTT | SEQ | PBS |
| SEQ ID NO: | SEQ ID NO: | SEQ ID NO: | Length | ID NO: | Length |
| 2557 | 33; 2293 | 2335 | 16 | 2295 | 8 |
| 2568 | 33; 2293 | 2339* | 16 | 2295 | 8 |
| 2871 | 33; 2293 | 2353 | 22 | 2295 | 8 |
| 2533 | 33; 2293 | 2320 | 13 | 2297 | 10 |
| 2615 | 33; 2293 | 2334* | 15 | 2297 | 10 |

²Indicated sequence does not contain the transcription adaptations used experimentally (i.e., addition of a 5′G if the spacer did not already start with a G and addition of 1-6 3′U from the U6 transcription termination sequence). The first 20 nt of the ngRNA sequence are the spacer; italics indicate that the spacer is a PE3B spacer.
³* = RTT contains a PAM silencing mutation.

TABLE XEb
PE3 screen at the H1069 mutation site in HEK293T cells

| | | PEgRNA² SEQ ID NO: | | | | | | | | | |
|---|---|---|---|---|---|---|---|---|---|---|---|
| | | 2633 | | 2585 | | 2667 | | 2744 | | 2890 | |
| | | % | % | % | % | % | % | % | % | % | % |
| | | Edit¹ | indel¹ | Edit¹ | indel¹ | Edit¹ | indel¹ | Edit¹ | indel¹ | Edit¹ | indel¹ |
| ngRNA² | PE2 | ++++ | + | ++++ | ++ | ++ | +++ | +++ | + | +++ | + |
| SEQ | 2263 | ++ | ++ | ++ | ++ | ++ | ++ | ++ | ++ | ++ | ++ |
| ID | 2266 | +++ | + | +++ | + | +++ | + | +++ | + | +++ | + |
| NO: | 2276 | ++ | +++ | ++ | +++ | ++ | +++ | ++ | ++ | ++ | +++ |
| | 2277 | ++++ | +++ | +++ | ++ | +++ | + | +++ | ++ | ++++ | ++ |
| | 2259 | ++++ | ++ | ++++ | ++ | ++++ | ++ | ++++ | ++ | ++++ | ++ |
| | 2257 | ++++ | +++ | ++++ | ++ | ++++ | ++ | ++++ | ++ | X | X |
| | 2268 | +++ | ++ | +++ | ++ | +++ | ++ | +++ | ++ | X | X |
| | 2264 | ++++ | + | ++++ | + | ++++ | + | +++ | + | X | X |
| | 2275 | ++++ | ++ | ++++ | ++ | ++++ | ++ | ++++ | ++ | ++++ | ++ |
| | 2273 | ++++ | +++ | ++++ | +++ | ++++ | +++ | ++++ | +++ | ++++ | ++ |
| | 2271 | +++ | +++ | +++ | +++ | +++ | +++ | +++ | +++ | ++++ | +++ |
| | 2261 | ++++ | +++ | ++++ | ++ | ++++ | +++ | ++++ | ++ | ++++ | ++ |
| | 2274 | ++++ | + | ++++ | + | ++++ | + | ++++ | + | ++++ | + |
| | 2269 | ++++ | + | ++++ | + | ++++ | + | ++++ | + | ++++ | + |
| | 2258 | +++ | ++++ | +++ | +++ | +++ | +++ | +++ | +++ | +++ | +++ |
| | 2270 | +++ | +++ | +++ | +++ | ++++ | +++ | +++ | +++ | ++++ | +++ |
| | 2262 | ++++ | +++ | ++++ | +++ | ++++ | +++ | ++++ | +++ | ++++ | ++ |
| | 2267 | ++++ | + | ++++ | + | ++++ | + | ++++ | + | ++++ | + |
| | 2265 | ++++ | + | ++++ | + | ++++ | + | +++ | + | ++++ | + |
| | 2260 | ++++ | + | ++++ | + | ++++ | + | ++++ | + | ++++ | + |
| | 2272 | ++++ | + | ++++ | + | ++++ | + | +++ | + | ++++ | + |
| | 4411 | ++++ | + | ++++ | + | ++++ | + | ++++ | + | ++++ | + |
| | 4418 | ++++ | +++ | ++++ | +++ | ++++ | +++ | ++++ | +++ | ++++ | ++ |
| | 4416 | ++++ | ++ | ++++ | + | ++++ | ++ | ++++ | ++ | + | + |

¹+ = 0.3%-6.3%; ++ = 6.3%-12.0%; +++ = 12%-20.3%; +++ = 20.3%-55.9%; X indicates successful PE3B editing was observed with the PEgRNA (data not shown)

### Summary of PEgRNA Used:

| | | | | PBS | |
|---|---|---|---|---|---|
| PEgRNA² | Spacer | RTT³ | RTT | SEQ | PBS |
| SEQ ID NO: | SEQ ID NO: | SEQ ID NO: | Length | ID NO: | Length |
| 2633 | 33; 2293 | 2335 | 16 | 2297 | 10 |
| 2585 | 33; 2293 | 2320 | 13 | 2299 | 12 |
| 2667 | 33; 2293 | 2329 | 15 | 2299 | 12 |
| 2744 | 33; 2293 | 2335 | 16 | 2299 | 12 |
| 2890 | 33; 2293 | 2344* | 18 | 2299 | 12 |

²Indicated sequence does not contain the transcription adaptations used experimentally (i.e., addition of a 5′G if the spacer did not already start with a G and addition of 1-6 3′U from the U6 transcription termination sequence). The first 20 nt of the ngRNA sequence are the spacer; italics indicate that the spacer is a PE3B spacer.
³* = RTT contains a PAM silencing mutation

TABLE XEc
PE3 screen at the H1069 mutation site in HEK293T cells

| | | PEgRNA² SEQ ID NO: | | | | | | | |
|---|---|---|---|---|---|---|---|---|---|
| | | 2675 | | 2729 | | 2898 | | 3165 | |
| | | % | % | % | % | % | % | % | % |
| | | Edit¹ | indel¹ | Edit¹ | indel¹ | Edit¹ | indel¹ | Edit¹ | indel¹ |
| ngRNA² | PE2 | ++++ | + | ++++ | ++ | +++ | ++ | ++++ | ++ |
| SEQ | 2263 | +++ | + | ++ | ++ | ++ | ++ | + | + |
| ID | 2266 | +++ | + | +++ | ++ | ++++ | + | ++ | + |
| NO: | 2276 | +++ | + | ++ | +++ | +++ | +++ | +++ | ++ |
| | 2277 | ++++ | + | +++ | +++ | ++++ | +++ | +++ | ++ |
| | 2259 | +++ | + | ++++ | ++ | ++++ | ++ | +++ | ++ |
| | 2257 | +++ | + | ++++ | ++ | ++++ | ++ | X | X |
| | 2268 | +++ | + | +++ | ++ | +++ | ++ | X | X |
| | 2264 | +++ | + | ++++ | + | ++++ | + | X | X |
| | 2275 | ++++ | + | ++++ | ++ | ++++ | ++ | ++++ | ++ |
| | 2273 | +++ | + | ++++ | +++ | ++++ | +++ | +++ | +++ |
| | 2271 | ++++ | + | +++ | +++ | ++++ | ++++ | +++ | +++ |
| | 2261 | +++ | + | ++++ | ++ | ++++ | +++ | +++ | +++ |
| | 2274 | ++++ | + | ++++ | + | ++++ | + | ++++ | + |
| | 2269 | ++++ | ++ | ++++ | + | ++++ | + | ++++ | ++ |
| | 2258 | ++++ | + | ++ | +++ | +++ | ++++ | ++ | +++ |
| | 2270 | ++++ | + | +++ | +++ | ++++ | +++ | +++ | ++ |
| | 2262 | ++++ | + | ++++ | ++ | ++++ | +++ | ++ | +++ |
| | 2267 | +++ | + | ++++ | + | ++++ | + | +++ | ++ |
| | 2265 | ++++ | + | ++++ | + | ++++ | + | ++ | + |
| | 2260 | ++++ | + | ++++ | + | ++++ | + | ++++ | + |
| | 2272 | + | + | ++++ | + | ++++ | + | +++ | + |
| | 4411 | +++ | + | ++++ | + | ++++ | + | +++ | ++ |
| | 4418 | +++ | + | ++++ | +++ | ++++ | +++ | ++ | +++ |
| | 4416 | ++++ | + | ++++ | + | ++++ | ++ | +++ | ++ |

¹+ = 0.3%-6.3%; ++ = 6.3%-12.0%; +++ = 12%-20.3%; +++ = 20.3%-55.9%; X indicates successful PE3B editing was observed with the PEgRNA (data not shown)

### Summary of PEgRNA Used:

| | | | | PBS | |
|---|---|---|---|---|---|
| PEgRNA² | Spacer | RTT³ | RTT | SEQ | PBS |
| SEQ ID NO: | SEQ ID NO: | SEQ ID NO: | Length | ID NO: | Length |
| 2675 | 33; 2293 | 2320 | 13 | 2301 | 14 |
| 2729 | 33; 2293 | 2326 | 14 | 2301 | 14 |
| 2898 | 33; 2293 | 2335 | 16 | 2301 | 14 |
| 3165 | 33; 2293 | 2344* | 18 | 2303 | 16 |

²Indicated sequence does not contain the transcription adaptations used experimentally (i.e., addition of a 5′G if the spacer did not already start with a G and addition of 1-6 3′U from the U6 transcription termination sequence). The first 20 nt of the ngRNA sequence are the spacer; italics indicate that the spacer is a PE3B spacer.
³* = RTT contains a PAM silencing mutation

TABLE XEd
PE3 screen at the H1069 mutation site in HEK293T cells

| | | PEgRNA² SEQ ID NO: | | | | | | | | | |
|---|---|---|---|---|---|---|---|---|---|---|---|
| | | 4540 | | 4756 | | 4624 | | 4592 | | 4691 | |
| | | % | % | % | % | % | % | % | % | % | % |
| | | Edit¹ | indel¹ | Edit¹ | indel¹ | Edit¹ | indel¹ | Edit¹ | indel¹ | Edit¹ | indel¹ |
| ngRNA² | PE2 | ++++ | +++ | +++ | ++ | ++++ | + | +++ | + | ++++ | + |
| SEQ | 2263 | ++ | + | + | + | ++ | + | ++ | + | ++ | + |
| ID | 2266 | +++ | + | ++ | + | +++ | + | +++ | + | +++ | + |
| NO: | 2276 | ++ | +++ | ++ | +++ | ++ | +++ | ++ | +++ | ++ | +++ |
| | 2277 | +++ | +++ | +++ | ++ | +++ | ++ | +++ | ++ | ++++ | +++ |
| | 2259 | ++++ | +++ | ++++ | ++ | ++++ | ++ | ++++ | ++ | ++++ | ++ |
| | 2257 | ++++ | +++ | ++++ | ++ | ++++ | ++ | ++++ | ++ | ++++ | +++ |
| | 2268 | +++ | ++ | +++ | + | +++ | + | ++++ | + | ++++ | ++ |
| | 2264 | ++++ | + | +++ | + | +++ | + | +++ | + | ++++ | + |
| | 2275 | ++++ | ++ | ++++ | + | ++++ | + | ++++ | + | ++++ | ++ |
| | 2273 | ++++ | +++ | ++++ | ++ | ++++ | ++ | ++++ | +++ | ++++ | +++ |
| | 2271 | +++ | +++ | +++ | +++ | +++ | +++ | +++ | +++ | +++ | +++ |
| | 2261 | ++++ | +++ | ++++ | ++ | ++++ | ++ | ++++ | ++ | ++++ | +++ |
| | 2274 | ++++ | + | ++++ | + | ++++ | + | ++++ | + | ++++ | + |
| | 2269 | ++++ | ++ | ++++ | + | ++++ | + | ++++ | + | ++++ | ++ |
| | 2258 | +++ | +++ | +++ | +++ | +++ | +++ | +++ | +++ | +++ | +++ |
| | 2270 | ++++ | +++ | +++ | ++ | +++ | +++ | ++++ | +++ | +++ | +++ |
| | 2262 | +++ | +++ | +++ | +++ | ++++ | ++ | ++++ | +++ | ++++ | +++ |
| | 2267 | ++++ | ++ | ++++ | + | ++++ | + | ++++ | + | ++++ | ++ |
| | 2265 | ++++ | + | +++ | + | +++ | + | +++ | + | ++++ | + |
| | 2260 | ++++ | ++ | ++++ | + | ++++ | + | ++++ | + | ++++ | ++ |
| | 2272 | ++++ | ++ | +++ | + | ++++ | + | ++++ | + | +++ | + |
| | 4411 | ++++ | ++ | ++++ | ++ | ++++ | + | ++++ | ++ | ++++ | ++ |
| | 4418 | ++++ | +++ | ++++ | ++ | ++++ | +++ | ++++ | +++ | ++++ | +++ |
| | 4416 | ++++ | +++ | ++++ | ++ | ++++ | ++ | ++++ | ++ | ++ | + |

¹+ = 0.3%-6.3%; ++ = 6.3%-12.0%; +++ = 12%-20.3%; +++ = 20.3%-55.9%

### Summary of PEgRNA Used:

| | | | | PBS | |
|---|---|---|---|---|---|
| PEgRNA² | Spacer | RTT | RTT | SEQ | PBS |
| SEQ ID NO: | SEQ ID NO: | SEQ ID NO: | Length | ID NO: | Length |
| 4540 | 58; 4425 | 4448 | 15 | 4427 | 8 |
| 4756 | 58; 4425 | 4456 | 21 | 4429 | 10 |
| 4624 | 58; 4425 | 4448 | 15 | 4431 | 12 |
| 4592 | 58; 4425 | 4441 | 12 | 4433 | 14 |
| 4691 | 58; 4425 | 4448 | 15 | 4433 | 14 |

²Indicated sequence does not contain the transcription adaptations used experimentally (i.e., addition of a 5′G if the spacer did not already start with a G and addition of 1-6 3′U from the U6 transcription termination sequence). The first 20 nt of the ngRNA sequence are the spacer; italics indicate that the spacer is a PE3B spacer.
³* = RTT contains a PAM silencing mutation

### Example 5—Prime Editing at a Lentivirus-Introduced ATP7B H1069Q Mutation Site in Primary Human Hepatocytes Using a PE3 System

PEgRNAs from the PE3 screen above were tested in hepatocytes. Primary human hepatocytes were transduced with lentivirus encoding the H1069Q cassette 2 days after cryorecovery, followed 6 days later by transfection with RNA encoding a prime editor, PEgRNA, and (where applicable) ngRNA. Genomic DNA was harvested after a 1-week incubation. Correction of the H1069Q mutation was examined and the results are provided in Table XD. “PET” in the ngRNA SEQ ID NO: column indicates a PE2 editing strategy was used instead of a PE3 editing strategy. These results demonstrate successful Prime Editing of the H1069 mutation site in clinically relevant cells types using both PE2 and PE3 editing strategies.

TABLE XF
PE3 screen at H1069Q mutation site in primary human hepatocytes

| PEgRNA¹ | | RTT | | PBS | | ngRNA¹ | | |
|---|---|---|---|---|---|---|---|---|
| SEQ ID | Spacer | SEQ ID | RTT | SEQ ID | PBS | SEQ ID | % | % |
| NO: | SEQ ID NO: | NO: | Len. | NO: | Len. | NO: | Edit | Indel |
| 2557 | 33; 2293 | 2335 | 16 | 2295 | 8 | PE2 | ++ | ++ |
| 2557 | 33; 2293 | 2335 | 16 | 2295 | 8 | 4414 | ++ | + |
| 2557 | 33; 2293 | 2335 | 16 | 2295 | 8 | 4412 | +++ | ++ |
| 2557 | 33; 2293 | 2335 | 16 | 2295 | 8 | 2269 | +++ | +++ |
| 2557 | 33; 2293 | 2335 | 16 | 2295 | 8 | 2260 | +++ | ++ |
| 2557 | 33; 2293 | 2335 | 16 | 2295 | 8 | PE2 | +++ | ++ |
| 2557 | 33; 2293 | 2335 | 16 | 2295 | 8 | 2268 | ++++ | ++ |
| 2557 | 33; 2293 | 2335 | 16 | 2295 | 8 | 2264 | ++++ | ++ |
| 2557 | 33; 2293 | 2335 | 16 | 2295 | 8 | 2269 | +++ | +++ |
| 2557 | 33; 2293 | 2335 | 16 | 2295 | 8 | 2260 | +++ | ++ |
| 4624 | 58; 4425 | 4448 | 15 | 4431 | 12 | PE2 | ++++ | + |
| 4624 | 58; 4425 | 4448 | 15 | 4431 | 12 | 2268 | + | + |
| 4624 | 58; 4425 | 4448 | 15 | 4431 | 12 | 2264 | ++++ | + |
| 4624 | 58; 4425 | 4448 | 15 | 4431 | 12 | 2269 | ++++ | +++ |
| 4624 | 58; 4425 | 4448 | 15 | 4431 | 12 | 2260 | ++++ | + |
| 2898 | 33; 2293 | 2335 | 16 | 2301 | 14 | PE2 | ++++ | + |
| 2898 | 33; 2293 | 2335 | 16 | 2301 | 14 | 2268 | ++++ | ++ |
| 2898 | 33; 2293 | 2335 | 16 | 2301 | 14 | 2264 | ++++ | + |
| 2898 | 33; 2293 | 2335 | 16 | 2301 | 14 | 2269 | ++++ | ++ |
| 2898 | 33; 2293 | 2335 | 16 | 2301 | 14 | 2260 | +++ | + |

¹Indicated PEgRNA or ngRNA sequence does not contain the adaptations for transcription from a DNA template used experimentally (i.e., addition of a 5′G if the spacer did not already start with a G and addition of 1-6 3′U from the U6 transcription termination sequence). The first 20 nts of the ngRNA sequence are the spacer; italics indicates the spacer is a PE3B spacer.
²+ = 0.03%-0.15%; ++ = 0.15%-0.55%; +++ = 0.55%-2.67%; ++++ = 2.67%-9.54%

### Example 6—Prime Editing at the Endogenous ATP7B H1069 Mutation Site in Mammalian Cells Using Synthetic PEgRNA in a PE2 System

An ATP7B H1069Q mutation was installed at the endogenous ATP7B locus in HEK293T and HepG2 cells by prime editing and single-cell clones were obtained via limiting dilution and clonal expansion, as described in Example 1. A PE2 screen measuring percent correction was performed at the endogenous ATP7B H1069Q locus. The cells were transfected with mRNA encoding a prime editor, and synthetic PEgRNA, as described in Example 1.

The results of the PE2 screen for the HEK and HepG2 are provided in Table XG. These data demonstrate successful Prime Editing at the endogenous ATP7B H1069Q mutation site in multiple mammalian cell models. Successful editing was observed with PEgRNAs containing multiple PBS lengths, multiple RTT lengths, and both with and without PAM silencing mutations.

These experiments were also performed in Huh cells (data not shown).

TABLE XG
PE2 screen at H1069Q mutation site in mammalian
cell culture using synthetic PEgRNAs

| PERNA¹ | | RTT² | | | | % Edit³ | % Edit⁴ |
|---|---|---|---|---|---|---|---|
| SEQ ID | Spacer | SEQ ID | RTT | PBS SEQ | PBS | in HEK | in HepG2 |
| NO: | SEQ ID NO: | NO: | Length | ID NO: | Length | cells | cells |
| 4495 | 58; 4425 | 4438 | 10 | 4427 | 8 | +++ | +++ |
| 4500 | 58; 4425 | 4440 | 11 | 4427 | 8 | ++++ | ++++ |
| 4504 | 58; 4425 | 4441 | 12 | 4427 | 8 | ++++ | ++ |
| 4517 | 58; 4425 | 4444 | 13 | 4427 | 8 | ++++ | +++ |
| 4522 | 58; 4425 | 4446 | 14 | 4427 | 8 | ++++ | + |
| 4540 | 58; 4425 | 4448 | 15 | 4427 | 8 | ++++ | ++++ |
| 4580 | 58; 4425 | 4450 | 17 | 4427 | 8 | ++++ | ++++ |
| 4591 | 58; 4425 | 4452 | 18 | 4427 | 8 | ++++ | +++ |
| 4667 | 58; 4425 | 4456 | 21 | 4427 | 8 | ++++ | +++ |
| 4499 | 58; 4425 | 4438 | 10 | 4428 | 9 | ++++ | ++++ |
| 4508 | 58; 4425 | 4440 | 11 | 4428 | 9 | +++ | |
| 4509 | 58; 4425 | 4441 | 12 | 4428 | 9 | ++++ | +++ |
| 4524 | 58; 4425 | 4444 | 13 | 4428 | 9 | ++++ | ++++ |
| 4531 | 58; 4425 | 4446 | 14 | 4428 | 9 | ++++ | ++++ |
| 4545 | 58; 4425 | 4448 | 15 | 4428 | 9 | ++++ | ++ |
| 4603 | 58; 4425 | 4450 | 17 | 4428 | 9 | ++++ | ++ |
| 4627 | 58; 4425 | 4452 | 18 | 4428 | 9 | ++++ | ++++ |
| 4710 | 58; 4425 | 4456 | 21 | 4428 | 9 | ++++ | +++ |
| 4507 | 58; 4425 | 4438 | 10 | 4429 | 10 | +++ | +++ |
| 4514 | 58; 4425 | 4440 | 11 | 4429 | 10 | ++++ | +++ |
| 4527 | 58; 4425 | 4441 | 12 | 4429 | 10 | +++ | ++++ |
| 4533 | 58; 4425 | 4444 | 13 | 4429 | 10 | ++++ | ++++ |
| 4555 | 58; 4425 | 4446 | 14 | 4429 | 10 | ++++ | +++ |
| 4564 | 58; 4425 | 4448 | 15 | 4429 | 10 | ++++ | ++++ |
| 4612 | 58; 4425 | 4450 | 17 | 4429 | 10 | ++++ | +++ |
| 4649 | 58; 4425 | 4452 | 18 | 4429 | 10 | ++++ | +++ |
| 4756 | 58; 4425 | 4456 | 21 | 4429 | 10 | ++++ | ++++ |
| 4510 | 58; 4425 | 4438 | 10 | 4430 | 11 | +++ | ++++ |
| 4529 | 58; 4425 | 4440 | 11 | 4430 | 11 | ++++ | ++++ |
| 4544 | 58; 4425 | 4441 | 12 | 4430 | 11 | ++++ | +++ |
| 4556 | 58; 4425 | 4444 | 13 | 4430 | 11 | ++++ | ++++ |
| 4579 | 58; 4425 | 4446 | 14 | 4430 | 11 | ++++ | ++++ |
| 4589 | 58; 4425 | 4448 | 15 | 4430 | 11 | ++++ | ++++ |
| 4634 | 58; 4425 | 4450 | 17 | 4430 | 11 | ++++ | ++++ |
| 4675 | 58; 4425 | 4452 | 18 | 4430 | 11 | ++++ | +++ |
| 4759 | 58; 4425 | 4456 | 21 | 4430 | 11 | ++++ | ++++ |
| 4519 | 58; 4425 | 4438 | 10 | 4431 | 12 | ++ | ++++ |
| 4542 | 58; 4425 | 4440 | 11 | 4431 | 12 | ++++ | +++ |
| 4559 | 58; 4425 | 4441 | 12 | 4431 | 12 | ++++ | +++ |
| 4576 | 58; 4425 | 4444 | 13 | 4431 | 12 | ++++ | +++ |
| 4588 | 58; 4425 | 4446 | 14 | 4431 | 12 | ++++ | ++++ |
| 4624 | 58; 4425 | 4448 | 15 | 4431 | 12 | ++++ | ++++ |
| 4679 | 58; 4425 | 4450 | 17 | 4431 | 12 | ++++ | ++++ |
| 4697 | 58; 4425 | 4452 | 18 | 4431 | 12 | +++ | ++++ |
| 4817 | 58; 4425 | 4456 | 21 | 4431 | 12 | +++ | +++ |
| 4532 | 58; 4425 | 4438 | 10 | 4432 | 13 | ++++ | ++++ |
| 4558 | 58; 4425 | 4440 | 11 | 4432 | 13 | +++ | ++++ |
| 4577 | 58; 4425 | 4441 | 12 | 4432 | 13 | ++++ | ++++ |
| 4605 | 58; 4425 | 4444 | 13 | 4432 | 13 | ++++ | ++++ |
| 4623 | 58; 4425 | 4446 | 14 | 4432 | 13 | ++++ | ++ |
| 4657 | 58; 4425 | 4448 | 15 | 4432 | 13 | ++++ | ++++ |
| 4719 | 58; 4425 | 4450 | 17 | 4432 | 13 | ++++ | |
| 4740 | 58; 4425 | 4452 | 18 | 4432 | 13 | ++++ | ++++ |
| 4827 | 58; 4425 | 4456 | 21 | 4432 | 13 | ++++ | ++++ |
| 4553 | 58; 4425 | 4438 | 10 | 4433 | 14 | +++ | ++ |
| 4562 | 58; 4425 | 4440 | 11 | 4433 | 14 | ++++ | ++++ |
| 4592 | 58; 4425 | 4441 | 12 | 4433 | 14 | ++++ | ++++ |
| 4618 | 58; 4425 | 4444 | 13 | 4433 | 14 | ++++ | +++ |
| 4640 | 58; 4425 | 4446 | 14 | 4433 | 14 | ++++ | +++ |
| 4691 | 58; 4425 | 4448 | 15 | 4433 | 14 | ++++ | +++ |
| 4726 | 58; 4425 | 4450 | 17 | 4433 | 14 | ++ | ++++ |
| 4761 | 58; 4425 | 4452 | 18 | 4433 | 14 | +++ | +++ |
| 4852 | 58; 4425 | 4456 | 21 | 4433 | 14 | ++ | ++++ |
| 4575 | 58; 4425 | 4438 | 10 | 4434 | 15 | ++ | ++++ |
| 4595 | 58; 4425 | 4440 | 11 | 4434 | 15 | +++ | +++ |
| 4619 | 58; 4425 | 4441 | 12 | 4434 | 15 | +++ | ++++ |
| 4638 | 58; 4425 | 4444 | 13 | 4434 | 15 | ++ | +++ |
| 4684 | 58; 4425 | 4446 | 14 | 4434 | 15 | ++ | +++ |
| 4720 | 58; 4425 | 4448 | 15 | 4434 | 15 | ++ | ++++ |
| 4758 | 58; 4425 | 4450 | 17 | 4434 | 15 | ++ | +++ |
| 4812 | 58; 4425 | 4452 | 18 | 4434 | 15 | ++ | ++++ |
| 4875 | 58; 4425 | 4456 | 21 | 4434 | 15 | + | +++ |
| 4586 | 58; 4425 | 4438 | 10 | 4435 | 16 | +++ | ++++ |
| 4629 | 58; 4425 | 4440 | 11 | 4435 | 16 | ++++ | ++++ |
| 4653 | 58; 4425 | 4441 | 12 | 4435 | 16 | ++++ | +++ |
| 4668 | 58; 4425 | 4444 | 13 | 4435 | 16 | ++++ | ++++ |
| 4724 | 58; 4425 | 4446 | 14 | 4435 | 16 | ++++ | ++++ |
| 4738 | 58; 4425 | 4448 | 15 | 4435 | 16 | ++++ | +++ |
| 4809 | 58; 4425 | 4450 | 17 | 4435 | 16 | ++++ | ++ |
| 4840 | 58; 4425 | 4452 | 18 | 4435 | 16 | + | ++++ |
| 4893 | 58; 4425 | 4456 | 21 | 4435 | 16 | ++++ | ++++ |
| 4614 | 58; 4425 | 4438 | 10 | 4436 | 17 | ++++ | +++ |
| 4655 | 58; 4425 | 4440 | 11 | 4436 | 17 | +++ | ++++ |
| 4674 | 58; 4425 | 4441 | 12 | 4436 | 17 | ++++ | +++ |
| 4722 | 58; 4425 | 4444 | 13 | 4436 | 17 | ++ | ++ |
| 4748 | 58; 4425 | 4446 | 14 | 4436 | 17 | ++ | +++ |
| 4785 | 58; 4425 | 4448 | 15 | 4436 | 17 | +++ | +++ |
| 4835 | 58; 4425 | 4450 | 17 | 4436 | 17 | +++ | +++ |
| 4856 | 58; 4425 | 4452 | 18 | 4436 | 17 | +++ | ++++ |
| 4912 | 58; 4425 | 4456 | 21 | 4436 | 17 | +++ | +++ |
| 2453 | 33; 2293 | 2307 | 11 | 2295 | 8 | ++ | ++ |
| 2480 | 33; 2293 | 2307 | 11 | 2297 | 10 | +++ | +++ |
| 2522 | 33; 2293 | 2307 | 11 | 2299 | 12 | +++ | +++ |
| 2606 | 33; 2293 | 2307 | 11 | 2301 | 14 | ++ | +++ |
| 2683 | 33; 2293 | 2307 | 11 | 2303 | 16 | +++ | +++ |
| 2459 | 33; 2293 | 2313 | 12 | 2295 | 8 | ++++ | ++++ |
| 2510 | 33; 2293 | 2313 | 12 | 2297 | 10 | ++++ | ++++ |
| 2575 | 33; 2293 | 2313 | 12 | 2299 | 12 | +++ | ++++ |
| 2623 | 33; 2293 | 2313 | 12 | 2301 | 14 | ++++ | ++++ |
| 2758 | 33; 2293 | 2313 | 12 | 2303 | 16 | ++++ | ++++ |
| 2489 | 33; 2293 | 2320 | 13 | 2295 | 8 | | ++++ |
| 2533 | 33; 2293 | 2320 | 13 | 2297 | 10 | | ++++ |
| 2585 | 33; 2293 | 2320 | 13 | 2299 | 12 | | ++++ |
| 2675 | 33; 2293 | 2320 | 13 | 2301 | 14 | +++ | +++ |
| 2825 | 33; 2293 | 2320 | 13 | 2303 | 16 | +++ | +++ |
| 2515 | 33; 2293 | 2326 | 14 | 2295 | 8 | +++ | +++ |
| 2559 | 33; 2293 | 2326 | 14 | 2297 | 10 | ++++ | ++++ |
| 2628 | 33; 2293 | 2326 | 14 | 2299 | 12 | +++ | ++++ |
| 2729 | 33; 2293 | 2326 | 14 | 2301 | 14 | ++++ | ++++ |
| 2844 | 33; 2293 | 2326 | 14 | 2303 | 16 | +++ | +++ |
| 2548 | 33; 2293 | 2329 | 15 | 2295 | 8 | ++++ | ++++ |
| 2605 | 33; 2293 | 2329 | 15 | 2297 | 10 | ++++ | ++++ |
| 2667 | 33; 2293 | 2329 | 15 | 2299 | 12 | ++++ | +++ |
| 2802 | 33; 2293 | 2329 | 15 | 2301 | 14 | ++++ | ++++ |
| 2932 | 33; 2293 | 2329 | 15 | 2303 | 16 | +++ | ++++ |
| 2557 | 33; 2293 | 2335 | 16 | 2295 | 8 | ++++ | +++ |
| 2633 | 33; 2293 | 2335 | 16 | 2297 | 10 | ++++ | ++++ |
| 2744 | 33; 2293 | 2335 | 16 | 2299 | 12 | ++++ | ++++ |
| 2898 | 33; 2293 | 2335 | 16 | 2301 | 14 | ++++ | +++ |
| 2993 | 33; 2293 | 2335 | 16 | 2303 | 16 | ++++ | ++++ |
| 2637 | 33; 2293 | 2342 | 18 | 2295 | 8 | ++++ | +++ |
| 2769 | 33; 2293 | 2342 | 18 | 2297 | 10 | ++++ | ++++ |
| 2886 | 33; 2293 | 2342 | 18 | 2299 | 12 | ++++ | +++ |
| 3043 | 33; 2293 | 2342 | 18 | 2301 | 14 | ++++ | ++++ |
| 3144 | 33; 2293 | 2342 | 18 | 2303 | 16 | ++++ | ++++ |
| 2689 | 33; 2293 | 2351 | 19 | 2295 | 8 | ++++ | +++ |
| 2801 | 33; 2293 | 2351 | 19 | 2297 | 10 | +++ | +++ |
| 2916 | 33; 2293 | 2351 | 19 | 2299 | 12 | +++ | +++ |
| 3076 | 33; 2293 | 2351 | 19 | 2301 | 14 | +++ | +++ |
| 3263 | 33; 2293 | 2351 | 19 | 2303 | 16 | +++ | +++ |
| 2871 | 33; 2293 | 2353 | 22 | 2295 | 8 | +++ | ++ |
| 2983 | 33; 2293 | 2353 | 22 | 2297 | 10 | +++ | +++ |
| 3163 | 33; 2293 | 2353 | 22 | 2299 | 12 | +++ | +++ |
| 3287 | 33; 2293 | 2353 | 22 | 2301 | 14 | +++ | +++ |
| 3441 | 33; 2293 | 2353 | 22 | 2303 | 16 | +++ | ++ |
| 2458 | 33; 2293 | 2310* | 11 | 2295 | 8 | + | ++ |
| 2487 | 33; 2293 | 2310* | 11 | 2297 | 10 | + | ++ |
| 2526 | 33; 2293 | 2310* | 11 | 2299 | 12 | + | ++ |
| 2609 | 33; 2293 | 2310* | 11 | 2301 | 14 | + | ++ |
| 2680 | 33; 2293 | 2310* | 11 | 2303 | 16 | + | + |
| 2466 | 33; 2293 | 2311* | 12 | 2295 | 8 | + | + |
| 2513 | 33; 2293 | 2311* | 12 | 2297 | 10 | + | ++ |
| 2554 | 33; 2293 | 2311* | 12 | 2299 | 12 | + | ++ |
| 2660 | 33; 2293 | 2311* | 12 | 2301 | 14 | + | ++ |
| 2759 | 33; 2293 | 2311* | 12 | 2303 | 16 | + | ++ |
| 2490 | 33; 2293 | 2321* | 13 | 2295 | 8 | + | ++ |
| 2531 | 33; 2293 | 2321* | 13 | 2297 | 10 | + | + |
| 2610 | 33; 2293 | 2321* | 13 | 2299 | 12 | ++ | ++ |
| 2685 | 33; 2293 | 2321* | 13 | 2301 | 14 | + | + |
| 2814 | 33; 2293 | 2321* | 13 | 2303 | 16 | + | ++ |
| 2512 | 33; 2293 | 2328* | 14 | 2295 | 8 | ++ | + |
| 2561 | 33; 2293 | 2328* | 14 | 2297 | 10 | + | ++ |
| 2643 | 33; 2293 | 2328* | 14 | 2299 | 12 | + | + |
| 2764 | 33; 2293 | 2328* | 14 | 2301 | 14 | + | + |
| 2843 | 33; 2293 | 2328* | 14 | 2303 | 16 | + | ++ |
| 2546 | 33; 2293 | 2334* | 15 | 2295 | 8 | ++ | ++ |
| 2615 | 33; 2293 | 2334* | 15 | 2297 | 10 | ++ | ++ |
| 2701 | 33; 2293 | 2334* | 15 | 2299 | 12 | ++ | +++ |
| 2777 | 33; 2293 | 2334* | 15 | 2301 | 14 | ++ | ++ |
| 2964 | 33; 2293 | 2334* | 15 | 2303 | 16 | ++ | ++ |
| 2568 | 33; 2293 | 2339* | 16 | 2295 | 8 | ++ | ++ |
| 2645 | 33; 2293 | 2339* | 16 | 2297 | 10 | ++ | ++ |
| 2735 | 33; 2293 | 2339* | 16 | 2299 | 12 | ++ | +++ |
| 2893 | 33; 2293 | 2339* | 16 | 2301 | 14 | +++ | +++ |
| 2988 | 33; 2293 | 2339* | 16 | 2303 | 16 | +++ | +++ |
| 2638 | 33; 2293 | 2343* | 18 | 2295 | 8 | +++ | ++ |
| 2772 | 33; 2293 | 2343* | 18 | 2297 | 10 | +++ | ++ |
| 2885 | 33; 2293 | 2343* | 18 | 2299 | 12 | +++ | +++ |
| 3015 | 33; 2293 | 2343* | 18 | 2301 | 14 | +++ | ++ |
| 3123 | 33; 2293 | 2343* | 18 | 2303 | 16 | +++ | ++ |
| 2690 | 33; 2293 | 2348* | 19 | 2295 | 8 | +++ | + |
| 2826 | 33; 2293 | 2348* | 19 | 2297 | 10 | +++ | +++ |
| 2960 | 33; 2293 | 2348* | 19 | 2299 | 12 | +++ | ++ |
| 3080 | 33; 2293 | 2348* | 19 | 2301 | 14 | +++ | ++ |
| 3248 | 33; 2293 | 2348* | 19 | 2303 | 16 | +++ | ++ |
| 2891 | 33; 2293 | 2355* | 22 | 2295 | 8 | ++ | ++ |
| 3012 | 33; 2293 | 2355* | 22 | 2297 | 10 | ++ | ++ |
| 3188 | 33; 2293 | 2355* | 22 | 2299 | 12 | ++ | +++ |
| 3301 | 33; 2293 | 2355* | 22 | 2301 | 14 | + | ++ |
| 3430 | 33; 2293 | 2355* | 22 | 2303 | 16 | ++ | ++ |
| 2457 | 33; 2293 | 2306* | 11 | 2295 | 8 | + | ++ |
| 2488 | 33; 2293 | 2306* | 11 | 2297 | 10 | + | ++ |
| 2544 | 33; 2293 | 2306* | 11 | 2299 | 12 | + | + |
| 2584 | 33; 2293 | 2306* | 11 | 2301 | 14 | + | ++ |
| 2702 | 33; 2293 | 2306* | 11 | 2303 | 16 | ++ | + |
| 2461 | 33; 2293 | 2316* | 12 | 2295 | 8 | + | ++ |
| 2493 | 33; 2293 | 2316* | 12 | 2297 | 10 | + | ++ |
| 2563 | 33; 2293 | 2316* | 12 | 2299 | 12 | ++ | |
| 2619 | 33; 2293 | 2316* | 12 | 2301 | 14 | + | |
| 2757 | 33; 2293 | 2316* | 12 | 2303 | 16 | + | |
| 2484 | 33; 2293 | 2319* | 13 | 2295 | 8 | ++ | |
| 2527 | 33; 2293 | 2319* | 13 | 2297 | 10 | ++ | |
| 2590 | 33; 2293 | 2319* | 13 | 2299 | 12 | +++ | |
| 2670 | 33; 2293 | 2319* | 13 | 2301 | 14 | ++ | |
| 2800 | 33; 2293 | 2319* | 13 | 2303 | 16 | +++ | |
| 2509 | 33; 2293 | 2325* | 14 | 2295 | 8 | ++ | |
| 2571 | 33; 2293 | 2325* | 14 | 2297 | 10 | ++ | |
| 2665 | 33; 2293 | 2325* | 14 | 2299 | 12 | +++ | |
| 2730 | 33; 2293 | 2325* | 14 | 2301 | 14 | ++ | |
| 2841 | 33; 2293 | 2325* | 14 | 2303 | 16 | ++ | ++ |
| 2541 | 33; 2293 | 2332* | 15 | 2295 | 8 | ++ | + |
| 2598 | 33; 2293 | 2332* | 15 | 2297 | 10 | ++++ | +++ |
| 2674 | 33; 2293 | 2332* | 15 | 2299 | 12 | +++ | ++ |
| 2832 | 33; 2293 | 2332* | 15 | 2301 | 14 | +++ | ++ |
| 2933 | 33; 2293 | 2332* | 15 | 2303 | 16 | +++ | ++ |
| 2551 | 33; 2293 | 2337* | 16 | 2295 | 8 | +++ | +++ |
| 2634 | 33; 2293 | 2337* | 16 | 2297 | 10 | +++ | ++ |
| 2733 | 33; 2293 | 2337* | 16 | 2299 | 12 | +++ | ++ |
| 2889 | 33; 2293 | 2337* | 16 | 2301 | 14 | +++ | +++ |
| 2979 | 33; 2293 | 2337* | 16 | 2303 | 16 | +++ | ++ |
| 2636 | 33; 2293 | 2344* | 18 | 2295 | 8 | +++ | + |
| 2765 | 33; 2293 | 2344* | 18 | 2297 | 10 | ++ | |
| 2890 | 33; 2293 | 2344* | 18 | 2299 | 12 | + | |
| 2982 | 33; 2293 | 2344* | 18 | 2301 | 14 | + | |
| 3165 | 33; 2293 | 2344* | 18 | 2303 | 16 | ++ | |
| 2706 | 33; 2293 | 2352* | 19 | 2295 | 8 | ++ | |
| 2807 | 33; 2293 | 2352* | 19 | 2297 | 10 | + | |
| 2962 | 33; 2293 | 2352* | 19 | 2299 | 12 | +++ | |
| 3072 | 33; 2293 | 2352* | 19 | 2301 | 14 | ++ | |
| 3210 | 33; 2293 | 2352* | 19 | 2303 | 16 | + | |
| 2894 | 33; 2293 | 2358* | 22 | 2295 | 8 | ++ | |
| 2977 | 33; 2293 | 2358* | 22 | 2297 | 10 | ++ | |
| 3166 | 33; 2293 | 2358* | 22 | 2299 | 12 | ++ | |
| 3300 | 33; 2293 | 2358* | 22 | 2301 | 14 | +++ | ++ |
| 3442 | 33; 2293 | 2358* | 22 | 2303 | 16 | +++ | + |
| 2451 | 33; 2293 | 2308* | 11 | 2295 | 8 | + | ++ |
| 2478 | 33; 2293 | 2308* | 11 | 2297 | 10 | + | ++ |
| 2528 | 33; 2293 | 2308* | 11 | 2299 | 12 | + | ++ |
| 2602 | 33; 2293 | 2308* | 11 | 2301 | 14 | + | ++ |
| 2711 | 33; 2293 | 2308* | 11 | 2303 | 16 | + | + |
| 2460 | 33; 2293 | 2312* | 12 | 2295 | 8 | ++ | + |
| 2514 | 33; 2293 | 2312* | 12 | 2297 | 10 | + | ++ |
| 2562 | 33; 2293 | 2312* | 12 | 2299 | 12 | + | ++ |
| 2647 | 33; 2293 | 2312* | 12 | 2301 | 14 | ++ | ++ |
| 2760 | 33; 2293 | 2312* | 12 | 2303 | 16 | + | ++ |
| 2473 | 33; 2293 | 2318* | 13 | 2295 | 8 | ++ | |
| 2520 | 33; 2293 | 2318* | 13 | 2297 | 10 | + | |
| 2596 | 33; 2293 | 2318* | 13 | 2299 | 12 | + | |
| 2677 | 33; 2293 | 2318* | 13 | 2301 | 14 | + | |
| 2830 | 33; 2293 | 2318* | 13 | 2303 | 16 | + | |
| 2501 | 33; 2293 | 2324* | 14 | 2295 | 8 | ++ | |
| 2553 | 33; 2293 | 2324* | 14 | 2297 | 10 | ++ | |
| 2646 | 33; 2293 | 2324* | 14 | 2299 | 12 | ++ | |
| 2749 | 33; 2293 | 2324* | 14 | 2301 | 14 | + | |
| 2842 | 33; 2293 | 2324* | 14 | 2303 | 16 | ++ | |
| 2535 | 33; 2293 | 2333* | 15 | 2295 | 8 | + | |
| 2618 | 33; 2293 | 2333* | 15 | 2297 | 10 | ++ | |
| 2699 | 33; 2293 | 2333* | 15 | 2299 | 12 | +++ | + |
| 2816 | 33; 2293 | 2333* | 15 | 2301 | 14 | +++ | ++ |
| 2926 | 33; 2293 | 2333* | 15 | 2303 | 16 | +++ | ++ |
| 2564 | 33; 2293 | 2338* | 16 | 2295 | 8 | +++ | ++ |
| 2632 | 33; 2293 | 2338* | 16 | 2297 | 10 | +++ | + |
| 2734 | 33; 2293 | 2338* | 16 | 2299 | 12 | +++ | +++ |
| 2834 | 33; 2293 | 2338* | 16 | 2301 | 14 | ++ | +++ |
| 2972 | 33; 2293 | 2338* | 16 | 2303 | 16 | ++ | + |
| 2635 | 33; 2293 | 2346* | 18 | 2295 | 8 | +++ | + |
| 2773 | 33; 2293 | 2346* | 18 | 2297 | 10 | +++ | ++ |
| 2899 | 33; 2293 | 2346* | 18 | 2299 | 12 | +++ | ++ |
| 3013 | 33; 2293 | 2346* | 18 | 2301 | 14 | +++ | + |
| 3148 | 33; 2293 | 2346* | 18 | 2303 | 16 | ++ | |
| 2669 | 33; 2293 | 2347* | 19 | 2295 | 8 | ++ | |
| 2829 | 33; 2293 | 2347* | 19 | 2297 | 10 | ++ | + |
| 2941 | 33; 2293 | 2347* | 19 | 2299 | 12 | ++ | + |
| 3071 | 33; 2293 | 2347* | 19 | 2301 | 14 | ++ | + |
| 3200 | 33; 2293 | 2347* | 19 | 2303 | 16 | ++ | + |
| 2856 | 33; 2293 | 2356* | 22 | 2295 | 8 | ++ | + |
| 3023 | 33; 2293 | 2356* | 22 | 2297 | 10 | ++ | + |
| 3122 | 33; 2293 | 2356* | 22 | 2299 | 12 | ++ | + |
| 3275 | 33; 2293 | 2356* | 22 | 2301 | 14 | ++ | + |
| 3420 | 33; 2293 | 2356* | 22 | 2303 | 16 | + | + |
| 1550 | 1505; 2055 | 1518 | 21 | 1507 | 8 | + | |
| 1565 | 1505; 2055 | 1518 | 21 | 1509 | 10 | + | + |
| 1578 | 1505; 2055 | 1518 | 21 | 1511 | 12 | + | + |
| 1602 | 1505; 2055 | 1518 | 21 | 1513 | 14 | + | + |
| 1640 | 1505; 2055 | 1518 | 21 | 1515 | 16 | + | + |
| 1554 | 1505; 2055 | 1519 | 22 | 1507 | 8 | + | + |
| 1569 | 1505; 2055 | 1519 | 22 | 1509 | 10 | + | + |
| 1591 | 1505; 2055 | 1519 | 22 | 1511 | 12 | + | + |
| 1626 | 1505; 2055 | 1519 | 22 | 1513 | 14 | + | + |
| 1654 | 1505; 2055 | 1519 | 22 | 1515 | 16 | + | + |
| 1562 | 1505; 2055 | 1522 | 23 | 1507 | 8 | + | + |
| 1580 | 1505; 2055 | 1522 | 23 | 1509 | 10 | + | + |
| 1609 | 1505; 2055 | 1522 | 23 | 1511 | 12 | + | + |
| 1628 | 1505; 2055 | 1522 | 23 | 1513 | 14 | | |
| 1691 | 1505; 2055 | 1522 | 23 | 1515 | 16 | | |
| 1570 | 1505; 2055 | 1524 | 24 | 1507 | 8 | | |
| 1593 | 1505; 2055 | 1524 | 24 | 1509 | 10 | | |
| 1616 | 1505; 2055 | 1524 | 24 | 1511 | 12 | | |
| 1664 | 1505; 2055 | 1524 | 24 | 1513 | 14 | | |
| 1708 | 1505; 2055 | 1524 | 24 | 1515 | 16 | | |
| 1583 | 1505; 2055 | 1526 | 25 | 1507 | 8 | | |
| 1600 | 1505; 2055 | 1526 | 25 | 1509 | 10 | | |
| 1643 | 1505; 2055 | 1526 | 25 | 1511 | 12 | | |
| 1671 | 1505; 2055 | 1526 | 25 | 1513 | 14 | | |
| 1725 | 1505; 2055 | 1526 | 25 | 1515 | 16 | | |
| 1551 | 1505; 2055 | 1517* | 21 | 1507 | 8 | + | + |
| 1564 | 1505; 2055 | 1517* | 21 | 1509 | 10 | + | + |
| 1586 | 1505; 2055 | 1517* | 21 | 1511 | 12 | + | + |
| 1606 | 1505; 2055 | 1517* | 21 | 1513 | 14 | + | + |
| 1646 | 1505; 2055 | 1517* | 21 | 1515 | 16 | + | + |
| 1553 | 1505; 2055 | 1520* | 22 | 1507 | 8 | + | + |
| 1575 | 1505; 2055 | 1520* | 22 | 1509 | 10 | + | + |
| 1587 | 1505; 2055 | 1520* | 22 | 1511 | 12 | + | + |
| 1615 | 1505; 2055 | 1520* | 22 | 1513 | 14 | + | + |
| 1663 | 1505; 2055 | 1520* | 22 | 1515 | 16 | + | + |
| 1566 | 1505; 2055 | 1521* | 23 | 1507 | 8 | + | + |
| 1577 | 1505; 2055 | 1521* | 23 | 1509 | 10 | ++ | + |
| 1607 | 1505; 2055 | 1521* | 23 | 1511 | 12 | + | |
| 1645 | 1505; 2055 | 1521* | 23 | 1513 | 14 | + | |
| 1678 | 1505; 2055 | 1521* | 23 | 1515 | 16 | ++ | |
| 1567 | 1505; 2055 | 1523* | 24 | 1507 | 8 | + | |
| 1590 | 1505; 2055 | 1523* | 24 | 1509 | 10 | ++ | |
| 1622 | 1505; 2055 | 1523* | 24 | 1511 | 12 | ++ | |
| 1668 | 1505; 2055 | 1523* | 24 | 1513 | 14 | +++ | |
| 1694 | 1505; 2055 | 1523* | 24 | 1515 | 16 | + | |
| 1584 | 1505; 2055 | 1525* | 25 | 1507 | 8 | +++ | |
| 1598 | 1505; 2055 | 1525* | 25 | 1509 | 10 | +++ | |
| 1637 | 1505; 2055 | 1525* | 25 | 1511 | 12 | ++ | |
| 1679 | 1505; 2055 | 1525* | 25 | 1513 | 14 | +++ | |
| 1721 | 1505; 2055 | 1525* | 25 | 1515 | 16 | +++ | + |
| 804 | 682; 2087 | 711 | 28 | 684 | 8 | +++ | + |
| 852 | 682; 2087 | 711 | 28 | 686 | 10 | ++ | + |
| 896 | 682; 2087 | 711 | 28 | 688 | 12 | ++ | + |
| 989 | 682; 2087 | 711 | 28 | 690 | 14 | ++ | + |
| 911 | 682; 2087 | 719 | 32 | 684 | 8 | ++ | + |
| 979 | 682; 2087 | 719 | 32 | 686 | 10 | ++ | + |
| 1046 | 682; 2087 | 719 | 32 | 688 | 12 | + | + |
| 1147 | 682; 2087 | 719 | 32 | 690 | 14 | + | + |
| 992 | 682; 2087 | 720 | 34 | 684 | 8 | ++ | + |
| 1045 | 682; 2087 | 720 | 34 | 686 | 10 | ++ | + |
| 1172 | 682; 2087 | 720 | 34 | 688 | 12 | ++ | + |
| 1244 | 682; 2087 | 720 | 34 | 690 | 14 | + | |
| 1124 | 682; 2087 | 724 | 37 | 684 | 8 | ++ | |
| 1220 | 682; 2087 | 724 | 37 | 686 | 10 | + | |
| 1270 | 682; 2087 | 724 | 37 | 688 | 12 | ++ | |
| 1342 | 682; 2087 | 724 | 37 | 690 | 14 | ++ | |

¹Indicated PEgRNA sequence does not contain the 3′ linker and hairpin motif used experimentally. The experimental PEgRNA further contained 3′ mN*mN*mN*N and 5′mN*mN*mN* modifications, where m indicates that the nucleotide contains a 2′-O-Me modification and a * indicates a phosphorothioate bond.
²* = RTT contains a PAM silencing mutation
³+ = 0.01%-0.77%, ++ = 0.77%-5.09%, +++ = 5.09%-15.96%, ++++ = 15.96%-45.89%
⁴+ = 0.01%-1.66%, ++ = 1.66%-2.62%, +++ = 2.62%-5.25%, ++++ = 5.25%-17.71%

### Example 7—Prime Editing with Silent Recoding at H1069Q Mutation Site

A PE2 screen measuring percent correction and percent indel formation was performed in HEK 293T at the endogenous ATP7B H1069Q locus. The cells were transfected with mRNA encoding a prime editor, and synthetic PEgRNA, as described in Example 1. Unlike previous examples, the PEgRNA used here were not designed to restore the wild-type nucleic acid sequence at the H1069Q site. Rather, the PEgRNA used here were designed to restore the wild-type amino acid sequence using an alternative histidine codon. The results presented in Table XH demonstrate successful recoding at the H1069Q mutation site using PEgRNA having multiple RTT and PBS length combinations.

TABLE XH
PE2 screen with silent recoding at H1069Q mutation site
in mammalian cell culture using synthetic PEgRNAs

| PEgRNA¹ | | | | | | | |
|---|---|---|---|---|---|---|---|
| SEQ ID | Spacer | RTT SEQ | RTT | PBS SEQ | PBS | | |
| NO: | SEQ ID NO: | ID NO: | Length | ID NO: | Length | % Edit² | % Indel² |
| 4494 | 58; 4425 | 4437 | 10 | 4427 | 8 | ++ | + |
| 4501 | 58; 4425 | 4439 | 11 | 4427 | 8 | +++ | + |
| 4503 | 58; 4425 | 4442 | 12 | 4427 | 8 | +++ | + |
| 4513 | 58; 4425 | 4443 | 13 | 4427 | 8 | +++ | + |
| 4525 | 58; 4425 | 4445 | 14 | 4427 | 8 | ++++ | + |
| 4530 | 58; 4425 | 4447 | 15 | 4427 | 8 | +++ | + |
| 4568 | 58; 4425 | 4451 | 17 | 4427 | 8 | +++ | + |
| 4597 | 58; 4425 | 4453 | 18 | 4427 | 8 | ++++ | ++ |
| 4682 | 58; 4425 | 4455 | 21 | 4427 | 8 | +++ | + |
| 4498 | 58; 4425 | 4437 | 10 | 4428 | 9 | +++ | + |
| 4505 | 58; 4425 | 4439 | 11 | 4428 | 9 | +++ | + |
| 4512 | 58; 4425 | 4442 | 12 | 4428 | 9 | ++++ | + |
| 4526 | 58; 4425 | 4443 | 13 | 4428 | 9 | ++++ | + |
| 4538 | 58; 4425 | 4445 | 14 | 4428 | 9 | ++++ | ++ |
| 4547 | 58; 4425 | 4447 | 15 | 4428 | 9 | ++++ | ++ |
| 4583 | 58; 4425 | 4451 | 17 | 4428 | 9 | ++++ | ++ |
| 4617 | 58; 4425 | 4453 | 18 | 4428 | 9 | ++++ | ++ |
| 4718 | 58; 4425 | 4455 | 21 | 4428 | 9 | ++++ | ++ |
| 4502 | 58; 4425 | 4437 | 10 | 4429 | 10 | +++ | + |
| 4511 | 58; 4425 | 4439 | 11 | 4429 | 10 | +++ | + |
| 4521 | 58; 4425 | 4442 | 12 | 4429 | 10 | +++ | + |
| 4543 | 58; 4425 | 4443 | 13 | 4429 | 10 | +++ | + |
| 4561 | 58; 4425 | 4445 | 14 | 4429 | 10 | ++++ | ++ |
| 4581 | 58; 4425 | 4447 | 15 | 4429 | 10 | ++++ | ++ |
| 4626 | 58; 4425 | 4451 | 17 | 4429 | 10 | ++++ | ++ |
| 4651 | 58; 4425 | 4453 | 18 | 4429 | 10 | ++++ | ++ |
| 4739 | 58; 4425 | 4455 | 21 | 4429 | 10 | +++ | ++ |
| 4515 | 58; 4425 | 4437 | 10 | 4430 | 11 | +++ | + |
| 4523 | 58; 4425 | 4439 | 11 | 4430 | 11 | +++ | + |
| 4537 | 58; 4425 | 4442 | 12 | 4430 | 11 | ++++ | + |
| 4557 | 58; 4425 | 4443 | 13 | 4430 | 11 | +++ | + |
| 4567 | 58; 4425 | 4445 | 14 | 4430 | 11 | ++++ | ++ |
| 4584 | 58; 4425 | 4447 | 15 | 4430 | 11 | ++++ | ++ |
| 4639 | 58; 4425 | 4451 | 17 | 4430 | 11 | ++++ | ++ |
| 4662 | 58; 4425 | 4453 | 18 | 4430 | 11 | ++++ | ++ |
| 4776 | 58; 4425 | 4455 | 21 | 4430 | 11 | +++ | ++ |
| 4528 | 58; 4425 | 4437 | 10 | 4431 | 12 | +++ | + |
| 4535 | 58; 4425 | 4439 | 11 | 4431 | 12 | ++++ | + |
| 4552 | 58; 4425 | 4442 | 12 | 4431 | 12 | ++++ | + |
| 4570 | 58; 4425 | 4443 | 13 | 4431 | 12 | ++++ | + |
| 4596 | 58; 4425 | 4445 | 14 | 4431 | 12 | +++ | +++ |
| 4621 | 58; 4425 | 4447 | 15 | 4431 | 12 | +++ | + |
| 4676 | 58; 4425 | 4451 | 17 | 4431 | 12 | ++++ | ++ |
| 4702 | 58; 4425 | 4453 | 18 | 4431 | 12 | +++ | + |
| 4795 | 58; 4425 | 4455 | 21 | 4431 | 12 | +++ | + |
| 4539 | 58; 4425 | 4437 | 10 | 4432 | 13 | +++ | + |
| 4546 | 58; 4425 | 4439 | 11 | 4432 | 13 | ++++ | + |
| 4573 | 58; 4425 | 4442 | 12 | 4432 | 13 | ++++ | + |
| 4585 | 58; 4425 | 4443 | 13 | 4432 | 13 | ++++ | ++ |
| 4630 | 58; 4425 | 4445 | 14 | 4432 | 13 | ++++ | ++ |
| 4660 | 58; 4425 | 4447 | 15 | 4432 | 13 | ++++ | +++ |
| 4714 | 58; 4425 | 4451 | 17 | 4432 | 13 | ++++ | ++ |
| 4751 | 58; 4425 | 4453 | 18 | 4432 | 13 | ++++ | ++ |
| 4820 | 58; 4425 | 4455 | 21 | 4432 | 13 | +++ | ++ |
| 4549 | 58; 4425 | 4437 | 10 | 4433 | 14 | +++ | + |
| 4574 | 58; 4425 | 4439 | 11 | 4433 | 14 | ++++ | ++ |
| 4582 | 58; 4425 | 4442 | 12 | 4433 | 14 | ++++ | ++ |
| 4625 | 58; 4425 | 4443 | 13 | 4433 | 14 | ++++ | + |
| 4646 | 58; 4425 | 4445 | 14 | 4433 | 14 | ++++ | ++ |
| 4670 | 58; 4425 | 4447 | 15 | 4433 | 14 | ++++ | ++ |
| 4725 | 58; 4425 | 4451 | 17 | 4433 | 14 | ++++ | +++ |
| 4784 | 58; 4425 | 4453 | 18 | 4433 | 14 | ++++ | ++ |
| 4857 | 58; 4425 | 4455 | 21 | 4433 | 14 | +++ | ++ |
| 4578 | 58; 4425 | 4437 | 10 | 4434 | 15 | +++ | + |
| 4604 | 58; 4425 | 4439 | 11 | 4434 | 15 | +++ | + |
| 4620 | 58; 4425 | 4442 | 12 | 4434 | 15 | ++++ | + |
| 4659 | 58; 4425 | 4443 | 13 | 4434 | 15 | +++ | + |
| 4677 | 58; 4425 | 4445 | 14 | 4434 | 15 | ++++ | ++ |
| 4717 | 58; 4425 | 4447 | 15 | 4434 | 15 | ++++ | ++ |
| 4760 | 58; 4425 | 4451 | 17 | 4434 | 15 | +++ | ++ |
| 4802 | 58; 4425 | 4453 | 18 | 4434 | 15 | +++ | ++ |
| 4874 | 58; 4425 | 4455 | 21 | 4434 | 15 | +++ | ++ |
| 4587 | 58; 4425 | 4437 | 10 | 4435 | 16 | ++ | + |
| 4606 | 58; 4425 | 4439 | 11 | 4435 | 16 | +++ | + |
| 4654 | 58; 4425 | 4442 | 12 | 4435 | 16 | ++++ | + |
| 4673 | 58; 4425 | 4443 | 13 | 4435 | 16 | +++ | + |
| 4698 | 58; 4425 | 4445 | 14 | 4435 | 16 | ++++ | + |
| 4737 | 58; 4425 | 4447 | 15 | 4435 | 16 | ++++ | ++ |
| 4804 | 58; 4425 | 4451 | 17 | 4435 | 16 | +++ | ++ |
| 4822 | 58; 4425 | 4453 | 18 | 4435 | 16 | +++ | ++ |
| 4902 | 58; 4425 | 4455 | 21 | 4435 | 16 | +++ | ++ |
| 4632 | 58; 4425 | 4437 | 10 | 4436 | 17 | ++ | + |
| 4656 | 58; 4425 | 4439 | 11 | 4436 | 17 | +++ | + |
| 4689 | 58; 4425 | 4442 | 12 | 4436 | 17 | ++++ | + |
| 4711 | 58; 4425 | 4443 | 13 | 4436 | 17 | +++ | + |
| 4746 | 58; 4425 | 4445 | 14 | 4436 | 17 | ++++ | ++ |
| 4780 | 58; 4425 | 4447 | 15 | 4436 | 17 | ++++ | ++ |
| 4841 | 58; 4425 | 4451 | 17 | 4436 | 17 | +++ | ++ |
| 4860 | 58; 4425 | 4453 | 18 | 4436 | 17 | +++ | ++ |
| 4915 | 58; 4425 | 4455 | 21 | 4436 | 17 | +++ | ++ |

¹Indicated PEgRNA sequence does not contain the 3′ mU*mU*mU*U and 5′mN*mN*mN* modifications used experimentally, where m indicates that the indicated nucleotide contains a 2′-O-Me modification and a * indicates a phosphorothioate bond.
²+ = 0-0.72%, ++ = 0.72%-2.33%, +++ = 2.33%-11.14%, ++++ = 11.14%-22.15%

### Example 8—Phenotypic Rescue by Prime Editing

Patient Fibroblast cells (GM05798) harboring homozygous H1069Q mutation were obtained from Coriell Institute. Fibroblast cells were propagated in EMEM with 15% FBS (not HI). 10 K cells were plated in 96-well plate and twenty-fours later cells were transfected with Messenger Max according to the manufacturer's directions with mRNA encoding a prime editor fusion protein, PEgRNA and NgRNA. Following transfection, the cells were challenged with copper (Cu) at a concentration of 500 uM. Twenty-four hours later, phenotypic rescue of the edited was measured by cell viability assay using cell titer glow from Promega according to the manufacture's protocol. The viability of the edited cells was normalized to the transfected cells with 0 Cu treatment and the phenotypic recue was measured relative to the untransfected cells challenged with the Cu at 500 uM. Editing of these cells were measured in parallel by harvesting cells in quick DNA extract for high throughput sequencing and sequenced using miseq.

The correlation between the percent correction and percent cell viability rescue data sets was analyzed assuming Gaussian distribution yielding Pearson correlation coefficients of 0.8970 with 95% confidence interval and R² value of 0.80. The P-value<0.0001 was calculated using 2-tailed test with 95% confidence interval. These data and analyses show that the level of phenotypic rescue in patient fibroblast cell populations correlates positively with percent correction observed in those populations.

TABLE XI
Phenotypic Rescue in Patient Fibroblast cells

| RNA Dose Level | Percent | Percent Cell |
|---|---|---|
| (1-high to 20-low) | Correction¹ | Viability Rescue² |
| 1 | ++++ | ++++ |
| 2 | ++++ | ++++ |
| 3 | ++++ | ++++ |
| 4 | ++++ | ++++ |
| 5 | ++++ | ++++ |
| 6 | ++++ | ++++ |
| 7 | +++ | +++ |
| 8 | +++ | +++ |
| 9 | +++ | ++ |
| 10 | +++ | +++ |
| 11 | +++ | +++ |
| 12 | ++ | ++ |
| 13 | ++ | + |
| 14 | ++ | ++ |
| 15 | ++ | + |
| 16 | ++ | ++ |
| 17 | + | + |
| 18 | + | +++ |
| 19 | + | ++ |
| 20 | + | + |
| 21 | + | + |

¹+ = 0.06-0.48%, ++ = 0.48%-3.84%, + ++ = 3.84%-30.05%, +++ + = 30.05%-47.97%
²+ = −10.3-5.14%, ++ = 5.14%-11.4%, +++ = 11.4%-31.58%, ++++ = 31.58%-55.66%

### Example 9—Prime Editing at the Endogenous ATP7B H1069 Mutation Site in HEK293T Cells Using Synthetic PEgRNA and ngRNA

A PE3 screen measuring percent correction and percent indels was performed at the endogenous ATP7B H1069Q locus. The cells were transfected with mRNA encoding a prime editor, and synthetic PEgRNA and ngRNA, as described in Example 1.

The results of the PE3 screen are provided in Tables XEa-XEe. Below each of Tables XEa-XEe is a table summarizing the PEgRNAs used experimentally. Each of the PEgRNA were tested in combination with multiple ngRNA. Some of the ngRNA were designed for a PE3B strategy and contain spacers complementary to the portion of the edit strand containing the edit. These results demonstrate the successful correction of the H1069Q mutation at the endogenous ATP7B locus in mammalian cells using both PE3 and PE3B Prime Editing systems.

TABLE Ya
PE3 screen at the H1069 mutation site in HEK293T
cells with synthetic PEgRNA and ngRNA

| | | PEgRNA² SEQ ID NO: | | | | | | | | | |
|---|---|---|---|---|---|---|---|---|---|---|---|
| | | 4499 | | 4533 | | 4522 | | 4518 | | 4540 | |
| | | % | % | % | % | % | % | % | % | % | % |
| | | Edit¹ | indel¹ | Edit¹ | indel¹ | Edit¹ | indel¹ | Edit¹ | indel¹ | Edit¹ | indel¹ |
| ngRNA² | PE2 | +++ | + | +++ | + | +++ | + | +++ | + | ++++ | + |
| SEQ | 2257 | +++ | + | ++++ | ++ | ++++ | + | +++ | ++ | ++++ | ++ |
| ID | 2268 | ++++ | ++ | ++++ | ++ | ++++ | ++ | ++++ | + | ++++ | + |
| NO: | 2264 | +++ | + | ++++ | + | ++++ | ++ | ++++ | + | ++++ | + |
| | 2261 | ++ | ++ | +++ | ++ | +++ | ++ | +++ | ++ | +++ | +++ |
| | 2274 | ++ | +++ | ++++ | +++ | +++ | +++ | +++ | ++ | ++++ | ++ |
| | 2269 | +++ | ++ | ++++ | +++ | +++ | +++ | +++ | ++ | +++ | ++ |
| | 2270 | ++ | ++ | ++++ | ++ | ++++ | ++ | +++ | + | ++++ | ++ |
| | 2265 | +++ | ++ | ++++ | ++ | ++++ | ++ | ++++ | ++ | ++++ | ++ |
| | 2260 | +++ | ++ | ++++ | ++ | ++++ | ++ | ++++ | ++ | ++++ | ++ |
| | 2272 | +++ | ++ | ++++ | ++ | ++++ | ++ | +++ | + | ++++ | ++ |
| | 4415 | ++ | + | ++++ | + | ++++ | + | +++ | + | +++ | + |
| | 4414 | +++ | + | ++++ | + | ++++ | + | ++++ | + | ++++ | + |
| | 4414 | ++++ | + | ++++ | + | ++++ | + | ++++ | + | ++++ | + |
| | 4412 | ++++ | + | ++++ | + | + | + | ++++ | + | ++++ | + |
| | 4412 | +++ | + | ++++ | + | ++++ | + | ++++ | + | ++++ | + |

4. + = 0.1%-2.0%; ++ = 2.0%-11.7%; +++ = 11.7%-34.85%; ++++ = 20.3%-55.9%

### Summary of PEgRNA Used:

| | | | | PBS | |
|---|---|---|---|---|---|
| PEgRNA² | Spacer | RTT³ | RTT | SEQ | PBS |
| SEQ ID NO: | SEQ ID NO: | SEQ ID NO: | Length | ID NO: | Length |
| 4499 | 58 | 4438 | 10 | 4428 | 9 |
| 4533 | 58 | 4444 | 13 | 4429 | 10 |
| 4522 | 58 | 4446 | 14 | 4427 | 8 |
| 4518 | 58 | 4448 | 15 | 4426 | 7 |
| 4540 | 58 | 4448 | 15 | 4427 | 8 |

5. Indicated sequence does not contain the 3′ linker and hairpin motif used experimentally. The experimental PEgRNA further contained 3′ mN*mN*mN*N and 5′mN*mN*mN* modifications, where m
indicates that the nucleotide contains a 2′-O—Me modification and a * indicates a phosphorothioate
bond .. The first 20 nt of the ngRNA sequence are the spacer; italics indicate that the spacer is a PE3B
spacer.
6. * = RTT contains a PAM silencing mutation.

TABLE Yb
PE3 screen at the H1069 mutation site in HEK293T
cells with synthetic PERNA and ngRNA

| | | PEgRNA² SEQ ID NO: | | | | | | | | | |
|---|---|---|---|---|---|---|---|---|---|---|---|
| | | 4657 | | 4691 | | 4560 | | # | | 4603 | |
| | | % | % | % | % | % | % | % | % | % | % |
| | | Edit¹ | indel¹ | Edit¹ | indel¹ | Edit¹ | indel¹ | Edit¹ | indel¹ | Edit¹ | indel¹ |
| ngRNA² | PE2 | +++ | + | +++ | + | +++ | + | +++ | + | ++++ | + |
| SEQ | 2257 | ++++ | ++ | ++++ | ++ | +++ | ++ | +++ | + | ++++ | ++ |
| ID | 2268 | ++++ | ++ | ++++ | ++ | ++++ | + | ++++ | + | ++++ | ++ |
| NO: | 2264 | ++++ | + | ++++ | + | ++++ | + | ++++ | + | ++++ | + |
| | 2261 | +++ | ++ | +++ | ++ | +++ | ++ | +++ | ++ | ++++ | ++ |
| | 2274 | +++ | ++ | +++ | ++ | +++ | ++ | +++ | ++ | ++++ | ++ |
| | 2269 | +++ | +++ | ++++ | +++ | +++ | ++ | +++ | ++ | +++ | ++ |
| | 2270 | ++++ | ++ | ++++ | ++ | ++++ | ++ | +++ | ++ | ++++ | ++ |
| | 2265 | ++++ | ++ | ++++ | ++ | ++++ | ++ | +++ | ++ | ++++ | ++ |
| | 2260 | ++++ | ++ | ++++ | ++ | ++++ | ++ | +++ | ++ | ++++ | ++ |
| | 2272 | ++++ | ++ | +++ | ++ | +++ | ++ | +++ | + | ++++ | ++ |
| | 4415 | ++++ | + | ++++ | + | +++ | + | +++ | + | ++++ | + |
| | 4414 | ++++ | + | ++++ | + | ++++ | + | ++++ | + | ++++ | + |
| | 4414 | ++++ | + | ++++ | + | ++++ | + | ++++ | + | ++++ | + |
| | 4412 | ++++ | + | ++++ | + | ++++ | + | ++++ | + | ++++ | + |
| | 4412 | ++++ | + | ++++ | + | ++++ | + | ++++ | + | ++++ | + |

¹+ = 0.1%-2.0%; ++ = 2.0%-11.7%; +++ = 11.7%-34.85%; ++++ = 20.3%-55.9%

### Summary of PEgRNA Used:

| | | | | PBS | |
|---|---|---|---|---|---|
| PEgRNA² | Spacer | RTT³ | RTT | SEQ | PBS |
| SEQ ID NO: | SEQ ID NO: | SEQ ID NO: | Length | ID NO: | Length |
| 4657 | 58 | 4448 | 15 | 4432 | 13 |
| 4691 | 58 | 4448 | 15 | 4433 | 14 |
| 4560 | 58 | 4449 | 16 | 4427 | 8 |
| # | 58 | 4449 | 16 | 4433 | 14 |
| 4603 | 58 | 4450 | 17 | 4428 | 9 |

²Indicated sequence does not contain the 3′ linker and hairpin motif used experimentally. The experimental PEgRNA further contained 3′ mN*mN*mN*N and 5′mN*mN*mN* modifications, where m indicates that the nucleotide contains a 2′-O—Me modification and a * indicates a phosphorothioate bond. The first 20 nt of the ngRNA sequence are the spacer; italics indicate that the spacer is a PE3B spacer.
³* = RTT contains a PAM silencing mutation.

TABLE Yc
PE3 screen at the H1069 mutation site in HEK293T
cells with synthetic PEgRNA and ngRNA

| | | PEgRNA² SEQ ID NO: | | | | | | | | | |
|---|---|---|---|---|---|---|---|---|---|---|---|
| | | 4719 | | 4649 | | 4592 | | 4592 | | 4624 | |
| | | % | % | % | % | % | % | % | % | % | % |
| | | Edit¹ | indel¹ | Edit¹ | indel¹ | Edit¹ | indel¹ | Edit¹ | indel¹ | Edit¹ | indel¹ |
| ngRNA² | PE2 | +++ | + | +++ | + | +++ | + | +++ | + | +++ | + |
| SEQ | 2257 | +++ | ++ | +++ | + | +++ | + | +++ | + | ++++ | ++ |
| ID | 2268 | ++++ | ++ | ++++ | + | ++++ | + | ++++ | ++ | ++++ | ++ |
| NO: | 2264 | ++++ | + | ++++ | + | ++++ | + | ++++ | + | ++++ | + |
| | 2261 | +++ | ++ | +++ | ++ | +++ | ++ | +++ | ++ | ++++ | +++ |
| | 2274 | +++ | ++ | +++ | ++ | +++ | ++ | +++ | ++ | +++ | +++ |
| | 2269 | +++ | ++ | +++ | ++ | | | +++ | ++ | +++ | +++ |
| | 2270 | +++ | + | +++ | ++ | +++ | + | +++ | ++ | ++++ | ++ |
| | 2265 | ++++ | ++ | ++++ | ++ | ++++ | ++ | +++ | ++ | ++++ | ++ |
| | 2260 | ++++ | ++ | ++++ | ++ | +++ | ++ | ++++ | ++ | ++++ | ++ |
| | 2272 | ++++ | ++ | +++ | + | +++ | ++ | +++ | ++ | ++++ | ++ |
| | 4415 | ++++ | + | +++ | + | | | | | ++++ | + |
| | 4414 | ++++ | + | ++++ | + | ++++ | + | ++++ | + | ++++ | + |
| | 4414 | ++++ | + | ++++ | ++ | ++++ | + | ++++ | + | ++++ | + |
| | 4412 | ++++ | + | ++++ | + | ++++ | + | ++++ | + | ++++ | + |
| | 4412 | ++++ | + | ++++ | + | ++++ | + | ++++ | + | ++++ | + |

¹+ = 0.1%-2.0%; ++ = 2.0%-11.7%; +++ = 11.7%-34.85%; ++++ = 20.3%-55.9%

### Summary of PEgRNA Used:

| | | | | PBS | |
|---|---|---|---|---|---|
| PEgRNA² | Spacer | RTT³ | RTT | SEQ | PBS |
| SEQ ID NO: | SEQ ID NO: | SEQ ID NO: | Length | ID NO: | Length |
| 4719 | 58 | 4450 | 17 | 4432 | 13 |
| 4649 | 58 | 4452 | 18 | 4429 | 10 |
| 4592 | 58 | 4441 | 12 | 4433 | 14 |
| 4592 | 58 | 4441 | 12 | 4433 | 14 |
| 4624 | 58 | 4448 | 15 | 4431 | 12 |

²Indicated sequence does not contain the 3′ linker and hairpin motif used experimentally. The experimental PEgRNA further contained 3′ mN*mN*mN*N and 5′mN*mN*mN* modifications, where m indicates that the nucleotide contains a 2′-O—Me modification and a * indicates a phosphorothioate bond. The first 20 nt of the ngRNA sequence are the spacer; italics indicate that the spacer is a PE3B spacer.
³* = RTT contains a PAM silencing mutation.

TABLE Yd
PE3 screen at the H1069 mutation site in HEK293T
cells with synthetic PEgRNA and ngRNA

| | | PEgRNA² SEQ ID NO: | | | | | | | | | |
|---|---|---|---|---|---|---|---|---|---|---|---|
| | | 4624 | | 4720 | | 4720 | | 4852 | | 4852 | |
| | | % | % | % | % | % | % | % | % | % | % |
| | | Edit¹ | indel¹ | Edit¹ | indel¹ | Edit¹ | indel¹ | Edit¹ | indel¹ | Edit¹ | indel¹ |
| ngRNA² | PE2 | +++ | + | +++ | + | +++ | + | +++ | + | +++ | + |
| SEQ | 2257 | +++ | ++ | +++ | ++ | +++ | ++ | +++ | ++ | +++ | + |
| ID | 2268 | ++++ | + | ++++ | ++ | ++++ | ++ | ++++ | + | ++++ | + |
| NO: | 2264 | ++++ | + | ++++ | + | ++++ | + | ++++ | + | ++++ | + |
| | 2261 | +++ | ++ | +++ | ++ | +++ | ++ | +++ | ++ | +++ | ++ |
| | 2274 | +++ | ++ | +++ | ++ | +++ | ++ | +++ | ++ | +++ | ++ |
| | 2269 | +++ | ++ | +++ | ++ | +++ | ++ | | | +++ | ++ |
| | 2270 | +++ | + | +++ | ++ | +++ | ++ | | | | |
| | 2265 | +++ | ++ | +++ | ++ | +++ | ++ | | | ++++ | ++ |
| | 2260 | ++++ | ++ | +++ | ++ | +++ | + | ++++ | ++ | ++++ | ++ |
| | 2272 | +++ | ++ | +++ | ++ | +++ | + | ++++ | ++ | +++ | ++ |
| | 4415 | +++ | + | +++ | + | +++ | + | | | | |
| | 4414 | ++++ | + | ++++ | + | ++++ | + | | | | |
| | 4414 | ++++ | + | ++++ | + | ++++ | + | ++++ | + | ++++ | + |
| | 4412 | ++++ | ++ | ++++ | + | ++++ | + | | | | |
| | 4412 | ++++ | + | ++++ | + | ++++ | + | ++++ | + | ++++ | + |

¹+ = 0.1%-2.0%; ++ = 2.0%-11.7%; +++ = 11.7%-34.85%; ++++ = 20.3%-55.9%

### Summary of PEgRNA Used:

| | | | | PBS | |
|---|---|---|---|---|---|
| PEgRNA² | Spacer | RTT³ | RTT | SEQ | PBS |
| SEQ ID NO: | SEQ ID NO: | SEQ ID NO: | Length | ID NO: | Length |
| 4624 | 58 | 4448 | 15 | 4431 | 12 |
| 4720 | 58 | 4448 | 15 | 4434 | 15 |
| 4720 | 58 | 4448 | 15 | 4434 | 15 |
| 4852 | 58 | 4456 | 21 | 4433 | 14 |
| 4852 | 58 | 4456 | 21 | 4433 | 14 |

²Indicated sequence does not contain the 3′ linker and hairpin motif used experimentally. The experimental PEgRNA further contained 3′ mN*mN*mN*N and 5′mN*mN*mN* modifications, where m indicates that the nucleotide contains a 2′-O—Me modification and a * indicates a phosphorothioate bond. The first 20 nt of the ngRNA sequence are the spacer; italics indicate that the spacer is a PE3B spacer.
³* = RTT contains a PAM silencing mutation.

TABLE Ye
PE3 screen at the H1069 mutation site in HEK293T
cells with synthetic PEgRNA and ngRNA

| | | PEgRNA² SEQ ID NO: | | | | | | | | | |
|---|---|---|---|---|---|---|---|---|---|---|---|
| | | 2548 | | 2557 | | 2582 | | 2633 | | ## | |
| | | % | % | % | % | % | % | % | % | % | % |
| | | Edit¹ | indel¹ | Edit¹ | indel¹ | Edit¹ | indel¹ | Edit¹ | indel¹ | Edit¹ | indel¹ |
| ngRNA² | PE2 | +++ | + | | | +++ | + | +++ | + | +++ | ++ |
| SEQ | 2257 | +++ | ++ | +++ | ++ | +++ | ++ | +++ | ++ | +++ | + |
| ID | 2268 | ++++ | + | ++++ | + | ++++ | ++ | ++++ | ++ | ++++ | + |
| NO: | 2264 | ++++ | + | ++++ | + | ++++ | + | ++++ | + | ++++ | + |
| | 2261 | +++ | ++ | +++ | ++ | +++ | ++ | +++ | ++ | +++ | ++ |
| | 2274 | +++ | ++ | +++ | ++ | +++ | ++ | +++ | ++ | +++ | ++ |
| | 2269 | +++ | ++ | +++ | ++ | +++ | ++ | +++ | +++ | +++ | ++ |
| | 2270 | +++ | ++ | +++ | ++ | +++ | + | +++ | ++ | +++ | + |
| | 2265 | +++ | ++ | +++ | ++ | +++ | ++ | +++ | ++ | +++ | + |
| | 2260 | +++ | ++ | +++ | ++ | +++ | ++ | +++ | ++ | +++ | ++ |
| | 2272 | +++ | ++ | +++ | + | +++ | ++ | +++ | ++ | +++ | ++ |
| | 4415 | +++ | + | +++ | + | +++ | + | +++ | ++ | +++ | + |
| | 4414 | ++++ | + | ++++ | + | ++++ | ++ | ++++ | + | +++ | + |
| | 4414 | ++++ | + | ++++ | + | | | ++++ | + | ++++ | + |
| | 4412 | ++++ | + | ++++ | + | ++++ | + | ++++ | + | +++ | + |
| | 4412 | +++ | + | ++++ | + | ++++ | + | ++++ | ++ | +++ | + |

¹+ = 0.1%-2.0%; ++ = 2.0%-11.7%; +++ = 11.7%-34.85%; ++++ = 20.3%-55.9%

### Summary of PEgRNA Used:

| | | | | PBS | |
|---|---|---|---|---|---|
| PEgRNA² | Spacer | RTT³ | RTT | SEQ | PBS |
| SEQ ID NO: | SEQ ID NO: | SEQ ID NO: | Length | ID NO: | Length |
| 2548 | 33 | 2329 | 15 | 2295 | 8 |
| 2557 | 33 | 2335 | 16 | 2295 | 8 |
| 2582 | 33 | 2335 | 16 | 2296 | 9 |
| 2633 | 33 | 2335 | 16 | 2297 | 10 |
| ## | 33 | ## | 17 | 2295 | 8 |

²Indicated sequence does not contain the 3′ linker and hairpin motif used experimentally. The experimental PEgRNA further contained 3′ mN*mN*mN*N and 5′mN*mN*mN* modifications, where m indicates that the nucleotide contains a 2′-O—Me modification and a * indicates a phosphorothioate bond. The first 20 nt of the ngRNA sequence are the spacer; italics indicate that the spacer is a PE3B spacer.
³* = RTT contains a PAM silencing mutation.

TABLE Yf
PE3 screen at the H1069 mutation site in HEK293T
cells with synthetic PEgRNA and ngRNA

| | | PEgRNA² SEQ ID NO: | | | | | | | | | |
|---|---|---|---|---|---|---|---|---|---|---|---|
| | | 2957 | | 2871 | | 2568 | | 2645 | | 2551 | |
| | | % | % | % | % | % | % | % | % | % | % |
| | | Edit¹ | indel¹ | Edit¹ | indel¹ | Edit¹ | indel¹ | Edit¹ | indel¹ | Edit¹ | indel¹ |
| ngRNA² | PE2 | +++ | + | | + | ++ | + | ++ | ++ | +++ | + |
| SEQ | 2257 | +++ | + | +++ | + | ++++ | + | +++ | ++ | ++++ | + |
| ID | 2268 | ++++ | ++ | +++ | | ++++ | + | | | ++++ | ++ |
| NO: | 2264 | ++++ | + | +++ | + | ++++ | + | +++ | + | ++++ | + |
| | 2261 | +++ | ++ | ++ | ++ | +++ | ++ | +++ | ++ | ++++ | ++ |
| | 2274 | +++ | ++ | ++ | ++ | +++ | ++ | +++ | ++ | +++ | ++ |
| | 2269 | +++ | ++ | +++ | ++ | +++ | ++ | +++ | ++ | ++++ | ++ |
| | 2270 | +++ | ++ | ++ | + | ++++ | ++ | +++ | ++ | ++++ | ++ |
| | 2265 | +++ | ++ | +++ | + | ++++ | ++ | +++ | ++ | ++++ | ++ |
| | 2260 | +++ | ++ | +++ | + | +++ | ++ | +++ | ++ | ++++ | ++ |
| | 2272 | +++ | ++ | +++ | ++ | +++ | + | +++ | ++ | +++ | ++ |
| | 4415 | +++ | + | ++ | + | +++ | + | +++ | + | +++ | + |
| | 4414 | ++++ | + | +++ | + | ++++ | ++ | ++++ | ++ | ++++ | + |
| | 4414 | ++++ | + | +++ | + | ++++ | + | +++ | + | ++++ | ++ |
| | 4412 | ++++ | + | +++ | + | ++++ | ++ | ++++ | ++ | ++++ | + |
| | 4412 | ++++ | + | +++ | + | +++ | + | +++ | + | ++++ | + |

¹+ = 0.1%-2.0%; ++ = 2.0%-11.7%; +++ = 11.7%-34.85%; ++++ = 20.3%-55.9%

### Summary of PEgRNA Used:

| | | | | PBS | |
|---|---|---|---|---|---|
| PEgRNA² | Spacer | RTT³ | RTT | SEQ | PBS |
| SEQ ID NO: | SEQ ID NO: | SEQ ID NO: | Length | ID NO: | Length |
| 2957 | 33 | 2342 | 18 | 2300 | 13 |
| 2871 | 33 | 2353 | 22 | 2295 | 8 |
| 2568 | 33 | 2339* | 16 | 2295 | 8 |
| 2645 | 33 | 2339* | 16 | 2297 | 10 |
| 2551 | 33 | 2337* | 16 | 2295 | 8 |

²Indicated sequence does not contain the 3′ linker and hairpin motif used experimentally. The experimental PEgRNA further contained 3′ mN*mN*mN*N and 5′mN*mN*mN* modifications, where m indicates that the nucleotide contains a 2′-O—Me modification and a * indicates a phosphorothioate bond. The first 20 nt of the ngRNA sequence are the spacer; italics indicate that the spacer is a PE3B spacer.
³* = RTT contains a PAM silencing mutation.

### Wild-Type ATP7B Protein Sequence (SEO ID NO: 5861)

>sp|P35670|ATP7B_HUMAN Copper-transporting
ATPase 2 OS = Homo sapiens OX = 9606
GN = ATP7B PE = 1 SV = 4
MPEQERQITAREGASRKILSKLSLPTRAWEPAMKKSFAFDNVGYEGGLDG
LGPSSQVATSTVRILGMTCQSCVKSIEDRISNLKGIISMKVSLEQGSATV
KYVPSVVCLQQVCHQIGDMGFEASIAEGKAASWPSRSLPAQEAVVKLRVE
GMTCQSCVSSIEGKVRKLQGVVRVKVSLSNQEAVITYQPYLIQPEDLRDH
VNDMGFEAAIKSKVAPLSLGPIDIERLQSTNPKRPLSSANQNFNNSETLG
HQGSHVVTLQLRIDGMHCKSCVLNIEENIGQLLGVQSIQVSLENKTAQVK
YDPSCTSPVALQRAIEALPPGNFKVSLPDGAEGSGTDHRSSSSHSPGSPP
RNQVQGTCSTTLIAIAGMTCASCVHSIEGMISQLEGVQQISVSLAEGTAT
VLYNPSVISPEELRAAIEDMGFEASVVSESCSTNPLGNHSAGNSMVQTTD
GTPTSVQEVAPHTGRLPANHAPDILAKSPQSTRAVAPQKCFLQIKGMTCA
SCVSNIERNLQKEAGVLSVLVALMAGKAEIKYDPEVIQPLEIAQFIQDLG
FEAAVMEDYAGSDGNIELTITGMTCASCVHNIESKLTRINGITYASVALA
TSKALVKFDPEIIGPRDIIKIIEEIGFHASLAQRNPNAHHLDHKMEIKQW
KKSFLCSLVFGIPVMALMIYMLIPSNEPHQSMVLDHNIIPGLSILNLIFF
ILCTFVQLLGGWYFYVQAYKSLRHRSANMDVLIVLATSIAYVYSLVILVV
AVAEKAERSPVTFFDTPPMLFVFIALGRWLEHLAKSKTSEALAKLMSLQA
TEATVVTLGEDNLIIREEQVPMELVQRGDIVKVVPGGKFPVDGKVLEGNT
MADESLITGEAMPVTKKPGSTVIAGSINAHGSVLIKATHVGNDTTLAQIV
KLVEEAQMSKAPIQQLADRFSGYFVPFIIIMSTLTLVVWIVIGFIDFGVV
QRYFPNPNKHISQTEVIIRFAFQTSITVLCIACPCSLGLATPTAVMVGTG
VAAQNGILIKGGKPLEMAHKIKTVMFDKTGTITHGVPRVMRVLLLGDVAT
LPLRKVLAVVGTAEASSEHPLGVAVTKYCKEELGTETLGYCTDFQAVPGC
GIGCKVSNVEGILAHSERPLSAPASHLNEAGSLPAEKDAVPQTFSVLIGN
REWLRRNGLTISSDVSDAMTDHEMKGQTAILVAIDGVLCGMIAIADAVKQ
EAALAVHTLQSMGVDVVLITGDNRKTARAIATQVGINKVFAEVLPSHKVA
KVQELQNKGKKVAMVGDGVNDSPALAQADMGVAIGTGTDVAIEAADVVLI
RNDLLDVVASIHLSKRTVRRIRINLVLALIYNLVGIPIAAGVFMPIGIVL
QPWMGSAAMAASSVSVVLSSLQLKCYKKPDLERYEAQAHGHMKPLTASQV
SVHIGMDDRWRDSPRATPWDQVSYVSQVSLSSLTSDKPSRHSAAADDDGD
KWSLLLNGRDEEQYI

1. A prime editing guide RNA (PEgRNA) comprising:
a. a spacer that is complementary to a search target sequence on a first strand of an ATP7B gene, wherein the spacer comprises at its 3′ end nucleotides 5-20 of SEQ ID NO: 4425;
b. a gRNA core capable of binding to a Cas9 protein;
c. an extension arm comprising:
i. an editing template that comprises a region of complementarity to an editing target sequence on a second strand of the ATP7B gene, and
ii. a primer binding site that comprises at its 5′ end a sequence that is a reverse complement of nucleotides 15-17 of SEQ ID NO: 4425;
wherein the first strand and second strand are complementary to each other and wherein the editing target sequence on the second strand is complementary to a portion of the ATP7B gene comprising a c.3207C>A substitution.

2. (canceled)

3. The PEgRNA of claim 1, wherein the spacer of the PEgRNA is from 15 to 22 nucleotides in length.

4. The PEgRNA of claim 1, wherein the spacer of the PEgRNA comprises at its 3′ end nucleotides 4-20, 3-20, 2-20, or 1-20 of SEQ ID NO: 4425.

5. The PEgRNA of claim 1, wherein the spacer of the PEgRNA comprises at its 3′ end SEQ ID NO: 4425.

6. The PEgRNA of claim 1, wherein the spacer of the PEgRNA is 20 nucleotides in length.

7. The PEgRNA of claim 1, comprising from 5′ to 3′, the spacer, the gRNA core, the RTT, and the PBS.

8. The PEgRNA of claim 7, wherein the spacer, the gRNA core, the RTT, and the PBS form a contiguous sequence in a single molecule.

9. The PEgRNA of claim 1, comprising a pegRNA sequence selected from any one of: SEQ ID NOs: 2445, 2446, 2447, 2448, 2449, 2450, 2452, 2453, 2454, 2455, 2456, 2457, 2458, 2459, 2460, 2461, 2462, 2463, 2464, 2465, 2466, 2467, 2468, 2469, 2470, 2471, 2472, 2473, 2474, 2475, 2476, 2477, 2478, 2479, 2480, 2481, 2482, 2483, 2484, 2485, 2486, 2487, 2488, 2489, 2490, 2491, 2492, 2493, 2494, 2495, 2496, 2497, 2498, 2499, 2500, 2501, 2502, 2503, 2504, 2505, 2506, 2507, 2508, 2509, 2510, 2511, 2512, 2513, 2514, 2515, 2516, 2517, 2518, 2519, 2520, 2521, 2522, 2523, 2524, 2525, 2526, 2527, 2528, 2529, 2530, 2531, 2532, 2533, 2534, 2535, 2538, 2539, 2540, 2541, 2542, 2543, 2544, 2545, 2546, 2547, 2548, 2549, 2550, 2551, 2553, 2554, 2555, 2556, 2557, 2558, 2559, 2560, 2561, 2562, 2563, 2564, 2565, 2566, 2567, 2568, 2569, 2570, 2571, 2572, 2573, 2574, 2575, 2576, 2577, 2578, 2580, 2582, 2584, 2585, 2586, 2587, 2588, 2589, 2590, 2591, 2592, 2593, 2594, 2595, 2596, 2597, 2598, 2600, 2601, 2602, 2603, 2605, 2606, 2607, 2608, 2609, 2610, 2611, 2612, 2613, 2614, 2615, 2616, 2617, 2618, 2619, 2620, 2621, 2623, 2624, 2628, 2629, 2630, 2631, 2632, 2633, 2634, 2635, 2636, 2637, 2638, 2643, 2645, 2646, 2647, 2648, 2649, 2650, 2651, 2652, 2653, 2654, 2655, 2656, 2657, 2658, 2659, 2660, 2663, 2664, 2665, 2667, 2668, 2669, 2670, 2671, 2672, 2674, 2675, 2676, 2677, 2678, 2680, 2681, 2683, 2685, 2687, 2688, 2689, 2690, 2692, 2694, 2695, 2696, 2697, 2699, 2701, 2702, 2704, 2706, 2708, 2711, 2713, 2715, 2716, 2717, 2720, 2721, 2722, 2723, 2725, 2726, 2727, 2728, 2729, 2730, 2733, 2734, 2735, 2744, 2747, 2748, 2749, 2752, 2753, 2757, 2758, 2759, 2760, 2761, 2762, 2764, 2765, 2768, 2769, 2770, 2772, 2773, 2774, 2777, 2786, 2788, 2789, 2790, 2791, 2792, 2793, 2794, 2795, 2796, 2797, 2798, 2799, 2800, 2801, 2802, 2807, 2810, 2811, 2812, 2814, 2816, 2824, 2825, 2826, 2828, 2829, 2830, 2832, 2833, 2834, 2841, 2842, 2843, 2844, 2846, 2847, 2854, 2855, 2856, 2857, 2862, 2864, 2866, 2867, 2868, 2869, 2870, 2871, 2885, 2886, 2887, 2888, 2889, 2890, 2891, 2893, 2894, 2896, 2898, 2899, 2901, 2902, 2909, 2910, 2914, 2916, 2918, 2919, 2920, 2926, 2927, 2932, 2933, 2937, 2938, 2939, 2941, 2942, 2945, 2953, 2954, 2956, 2957, 2960, 2962, 2963, 2964, 2965, 2967, 2972, 2973, 2977, 2979, 2980, 2982, 2983, 2988, 2991, 2993, 2994, 2995, 2997, 3006, 3008, 3012, 3013, 3015, 3023, 3024, 3027, 3028, 3029, 3030, 3031, 3032, 3033, 3034, 3035, 3036, 3037, 3038, 3039, 3043, 3044, 3045, 3046, 3048, 3049, 3050, 3051, 3052, 3053, 3054, 3055, 3059, 3064, 3065, 3071, 3072, 3075, 3076, 3080, 3082, 3084, 3093, 3096, 3098, 3099, 3101, 3119, 3121, 3122, 3123, 3124, 3126, 3128, 3130, 3133, 3142, 3144, 3148, 3159, 3161, 3162, 3163, 3164, 3165, 3166, 3168, 3169, 3170, 3176, 3182, 3188, 3190, 3191, 3195, 3200, 3202, 3203, 3210, 3212, 3216, 3218, 3226, 3227, 3228, 3229, 3230, 3231, 3232, 3234, 3235, 3238, 3239, 3241, 3243, 3244, 3245, 3246, 3247, 3248, 3249, 3250, 3260, 3262, 3263, 3271, 3273, 3275, 3281, 3282, 3283, 3287, 3288, 3289, 3300, 3301, 3302, 3303, 3304, 3305, 3307, 3310, 3311, 3312, 3313, 3314, 3315, 3316, 3317, 3318, 3322, 3324, 3325, 3328, 3330, 3346, 3347, 3348, 3349, 3350, 3358, 3359, 3362, 3364, 3365, 3366, 3367, 3368, 3372, 3373, 3382, 3385, 3387, 3388, 3389, 3390, 3391, 3392, 3393, 3400, 3403, 3404, 3405, 3407, 3408, 3409, 3412, 3414, 3420, 3423, 3425, 3426, 3427, 3428, 3429, 3430, 3431, 3434, 3438, 3441, 3442, 3446, 3449, 3450, 3451, 3452, 3453, 3454, 3455, 3463, 3466, 3469, 3470, 3471, 3472, 3473, 3474, 3477, 3478, 3480, 3481, 3482, 3487, 3490, 3494, 3498, 3499, 3502, 3503, 3505, 3506, 3508, 3509, 3510, 3511, 3513, 3520, 3522, 3523, 3526, 3529, 3533, 3535, 3536, 3542, 3543, 3546, 3547, 3549, 3550, 3553, 3554, 3555, 3557, 3560, 3561, 3563, 3564, 3567, 3568, 3569, 3571, 3574, 3575, 3576, 3578, 3579, 3580, 3581, 3583, 3584, 3585, 3592, 3594, 3595, 3596, 3597, 3603, 3612, 3613, 3617, 3622, 3625, 3626, 3627, 3628, 3630, 3631, 3632, 3633, 3635, 3636, 3638, 3639, 3640, 3641, 3642, 3646, 3647, 3648, 3654, 3657, 3659, 3660, 3661, 3664, 3668, 3669, 3673, 3674, 3678, 3679, 3680, 3681, 3684, 3685, 3687, 3688, 3697, 3699, 3702, 3703, 3704, 3705, 3706, 3708, 3710, 3711, 3712, 3714, 3715, 3721, 3722, 3724, 3725, 3728, 3729, 3730, 3731, 3732, 3733, 3734, 3735, 3736, 3737, 3739, 3740, 3741, 3743, 3744, 3746, 3748, 3755, 3761, 3770, 3771, 3773, 3774, 3776, 3778, 3779, 3781, 3782, 3784, 3785, 3792, 3793, 3794, 3795, 3796, 3797, 3798, 3799, 3800, 3801, 3802, 3803, 3804, 3805, 3806, 3807, 3808, 3809, 3810, 3811, 3812, 3814, 3815, 3816, 3820, 3829, 3839, 3841, 3842, 3843, 3844, 3845, 3851, 3852, 3853, 3854, 3855, 3856, 3857, 3858, 3859, 3860, 3861, 3862, 3863, 3864, 3865, 3868, 3869, 3871, 3874, 3875, 3876, 3877, 3878, 3879, 3880, 3882, 3883, 3884, 3885, 3887, 3895, 3899, 3904, 3907, 3908, 3909, 3910, 3911, 3912, 3913, 3914, 3915, 3916, 3917, 3921, 3924, 3927, 3928, 3929, 3931, 3932, 3935, 3936, 3937, 3938, 3939, 3940, 3941, 3942, 3943, 3945, 3946, 3956, 3957, 3961, 3962, 3965, 3971, 3977, 3978, 3979, 3980, 3981, 3982, 3983, 3985, 3988, 3989, 3990, 3991, 3992, 3993, 3994, 3995, 3997, 3998, 3999, 4001, 4002, 4003, 4004, 4009, 4011, 4012, 4013, 4015, 4016, 4017, 4020, 4021, 4023, 4025, 4026, 4028, 4029, 4031, 4032, 4034, 4035, 4036, 4037, 4038, 4040, 4052, 4055, 4056, 4060, 4061, 4066, 4067, 4070, 4077, 4078, 4080, 4081, 4082, 4083, 4084, 4085, 4086, 4087, 4088, 4089, 4090, 4102, 4105, 4106, 4108, 4109, 4110, 4114, 4115, 4117, 4118, 4119, 4128, 4129, 4132, 4136, 4137, 4142, 4147, 4159, 4163, 4168, 4170, 4171, 4172, 4173, 4175, 4182, 4183, 4186, 4188, 4192, 4194, 4199, 4208, 4225, 4226, 4227, 4228, 4232, 4239, 4240, or 4258.

10. The PEgRNA of claim 1, comprising a pegRNA sequence selected from any one of SEQ ID NOs: 4588, 4657, 4719, 4589, 4624, 4500, 4618, 4649, or 4533.

11. A prime editing guide RNA (PEgRNA) comprising:
a. a spacer that is complementary to a search target sequence on a first strand of an ATP7B gene, wherein the spacer comprises at its 3′ end nucleotides 5-20 of SEQ ID NO: 2293;
b. a gRNA core capable of binding to a Cas9 protein;
c. an extension arm comprising:
i. an editing template that comprises a region of complementarity to an editing target sequence on a second strand of the ATP7B gene, and
ii. a primer binding site that comprises at its 5′ end a sequence that is a reverse complement of nucleotides 15-17 of SEQ ID NO: 2293;
wherein the first strand and second strand are complementary to each other and wherein the editing target sequence on the second strand is complementary to a portion of the ATP7B gene comprising a c.3207C>A substitution.

12. (canceled)

13. The PEgRNA of claim 11, wherein the spacer of the PEgRNA is from 15 to 22 nucleotides in length.

14. The PEgRNA of claim 11, wherein the spacer of the PEgRNA comprises at its 3′ end nucleotides 4-20, 3-20, 2-20, or 1-20 of SEQ ID NO: 2293.

15. The PEgRNA of claim 11, wherein the spacer of the PEgRNA comprises at its 3′ end SEQ ID NO: 2293.

16. The PEgRNA of claim 11, wherein the spacer of the PEgRNA is 20 nucleotides in length.

17. The PEgRNA of claim 11, comprising from 5′ to 3′, the spacer, the gRNA core, the RTT, and the PBS.

18. The PEgRNA of claim 17, wherein the spacer, the gRNA core, the RTT, and the PBS form a contiguous sequence in a single molecule.

19. The PEgRNA of claim 17, comprising a pegRNA sequence selected from any one of: SEQ ID NOs: 2445, 2446, 2447, 2448, 2449, 2450, 2452, 2453, 2454, 2455, 2456, 2457, 2458, 2459, 2460, 2461, 2462, 2463, 2464, 2465, 2466, 2467, 2468, 2469, 2470, 2471, 2472, 2473, 2474, 2475, 2476, 2477, 2478, 2479, 2480, 2481, 2482, 2483, 2484, 2485, 2486, 2487, 2488, 2489, 2490, 2491, 2492, 2493, 2494, 2495, 2496, 2497, 2498, 2499, 2500, 2501, 2502, 2503, 2504, 2505, 2506, 2507, 2508, 2509, 2510, 2511, 2512, 2513, 2514, 2515, 2516, 2517, 2518, 2519, 2520, 2521, 2522, 2523, 2524, 2525, 2526, 2527, 2528, 2529, 2530, 2531, 2532, 2533, 2534, 2535, 2538, 2539, 2540, 2541, 2542, 2543, 2544, 2545, 2546, 2547, 2548, 2549, 2550, 2551, 2553, 2554, 2555, 2556, 2557, 2558, 2559, 2560, 2561, 2562, 2563, 2564, 2565, 2566, 2567, 2568, 2569, 2570, 2571, 2572, 2573, 2574, 2575, 2576, 2577, 2578, 2580, 2582, 2584, 2585, 2586, 2587, 2588, 2589, 2590, 2591, 2592, 2593, 2594, 2595, 2596, 2597, 2598, 2600, 2601, 2602, 2603, 2605, 2606, 2607, 2608, 2609, 2610, 2611, 2612, 2613, 2614, 2615, 2616, 2617, 2618, 2619, 2620, 2621, 2623, 2624, 2628, 2629, 2630, 2631, 2632, 2633, 2634, 2635, 2636, 2637, 2638, 2643, 2645, 2646, 2647, 2648, 2649, 2650, 2651, 2652, 2653, 2654, 2655, 2656, 2657, 2658, 2659, 2660, 2663, 2664, 2665, 2667, 2668, 2669, 2670, 2671, 2672, 2674, 2675, 2676, 2677, 2678, 2680, 2681, 2683, 2685, 2687, 2688, 2689, 2690, 2692, 2694, 2695, 2696, 2697, 2699, 2701, 2702, 2704, 2706, 2708, 2711, 2713, 2715, 2716, 2717, 2720, 2721, 2722, 2723, 2725, 2726, 2727, 2728, 2729, 2730, 2733, 2734, 2735, 2744, 2747, 2748, 2749, 2752, 2753, 2757, 2758, 2759, 2760, 2761, 2762, 2764, 2765, 2768, 2769, 2770, 2772, 2773, 2774, 2777, 2786, 2788, 2789, 2790, 2791, 2792, 2793, 2794, 2795, 2796, 2797, 2798, 2799, 2800, 2801, 2802, 2807, 2810, 2811, 2812, 2814, 2816, 2824, 2825, 2826, 2828, 2829, 2830, 2832, 2833, 2834, 2841, 2842, 2843, 2844, 2846, 2847, 2854, 2855, 2856, 2857, 2862, 2864, 2866, 2867, 2868, 2869, 2870, 2871, 2885, 2886, 2887, 2888, 2889, 2890, 2891, 2893, 2894, 2896, 2898, 2899, 2901, 2902, 2909, 2910, 2914, 2916, 2918, 2919, 2920, 2926, 2927, 2932, 2933, 2937, 2938, 2939, 2941, 2942, 2945, 2953, 2954, 2956, 2957, 2960, 2962, 2963, 2964, 2965, 2967, 2972, 2973, 2977, 2979, 2980, 2982, 2983, 2988, 2991, 2993, 2994, 2995, 2997, 3006, 3008, 3012, 3013, 3015, 3023, 3024, 3027, 3028, 3029, 3030, 3031, 3032, 3033, 3034, 3035, 3036, 3037, 3038, 3039, 3043, 3044, 3045, 3046, 3048, 3049, 3050, 3051, 3052, 3053, 3054, 3055, 3059, 3064, 3065, 3071, 3072, 3075, 3076, 3080, 3082, 3084, 3093, 3096, 3098, 3099, 3101, 3119, 3121, 3122, 3123, 3124, 3126, 3128, 3130, 3133, 3142, 3144, 3148, 3159, 3161, 3162, 3163, 3164, 3165, 3166, 3168, 3169, 3170, 3176, 3182, 3188, 3190, 3191, 3195, 3200, 3202, 3203, 3210, 3212, 3216, 3218, 3226, 3227, 3228, 3229, 3230, 3231, 3232, 3234, 3235, 3238, 3239, 3241, 3243, 3244, 3245, 3246, 3247, 3248, 3249, 3250, 3260, 3262, 3263, 3271, 3273, 3275, 3281, 3282, 3283, 3287, 3288, 3289, 3300, 3301, 3302, 3303, 3304, 3305, 3307, 3310, 3311, 3312, 3313, 3314, 3315, 3316, 3317, 3318, 3322, 3324, 3325, 3328, 3330, 3346, 3347, 3348, 3349, 3350, 3358, 3359, 3362, 3364, 3365, 3366, 3367, 3368, 3372, 3373, 3382, 3385, 3387, 3388, 3389, 3390, 3391, 3392, 3393, 3400, 3403, 3404, 3405, 3407, 3408, 3409, 3412, 3414, 3420, 3423, 3425, 3426, 3427, 3428, 3429, 3430, 3431, 3434, 3438, 3441, 3442, 3446, 3449, 3450, 3451, 3452, 3453, 3454, 3455, 3463, 3466, 3469, 3470, 3471, 3472, 3473, 3474, 3477, 3478, 3480, 3481, 3482, 3487, 3490, 3494, 3498, 3499, 3502, 3503, 3505, 3506, 3508, 3509, 3510, 3511, 3513, 3520, 3522, 3523, 3526, 3529, 3533, 3535, 3536, 3542, 3543, 3546, 3547, 3549, 3550, 3553, 3554, 3555, 3557, 3560, 3561, 3563, 3564, 3567, 3568, 3569, 3571, 3574, 3575, 3576, 3578, 3579, 3580, 3581, 3583, 3584, 3585, 3592, 3594, 3595, 3596, 3597, 3603, 3612, 3613, 3617, 3622, 3625, 3626, 3627, 3628, 3630, 3631, 3632, 3633, 3635, 3636, 3638, 3639, 3640, 3641, 3642, 3646, 3647, 3648, 3654, 3657, 3659, 3660, 3661, 3664, 3668, 3669, 3673, 3674, 3678, 3679, 3680, 3681, 3684, 3685, 3687, 3688, 3697, 3699, 3702, 3703, 3704, 3705, 3706, 3708, 3710, 3711, 3712, 3714, 3715, 3721, 3722, 3724, 3725, 3728, 3729, 3730, 3731, 3732, 3733, 3734, 3735, 3736, 3737, 3739, 3740, 3741, 3743, 3744, 3746, 3748, 3755, 3761, 3770, 3771, 3773, 3774, 3776, 3778, 3779, 3781, 3782, 3784, 3785, 3792, 3793, 3794, 3795, 3796, 3797, 3798, 3799, 3800, 3801, 3802, 3803, 3804, 3805, 3806, 3807, 3808, 3809, 3810, 3811, 3812, 3814, 3815, 3816, 3820, 3829, 3839, 3841, 3842, 3843, 3844, 3845, 3851, 3852, 3853, 3854, 3855, 3856, 3857, 3858, 3859, 3860, 3861, 3862, 3863, 3864, 3865, 3868, 3869, 3871, 3874, 3875, 3876, 3877, 3878, 3879, 3880, 3882, 3883, 3884, 3885, 3887, 3895, 3899, 3904, 3907, 3908, 3909, 3910, 3911, 3912, 3913, 3914, 3915, 3916, 3917, 3921, 3924, 3927, 3928, 3929, 3931, 3932, 3935, 3936, 3937, 3938, 3939, 3940, 3941, 3942, 3943, 3945, 3946, 3956, 3957, 3961, 3962, 3965, 3971, 3977, 3978, 3979, 3980, 3981, 3982, 3983, 3985, 3988, 3989, 3990, 3991, 3992, 3993, 3994, 3995, 3997, 3998, 3999, 4001, 4002, 4003, 4004, 4009, 4011, 4012, 4013, 4015, 4016, 4017, 4020, 4021, 4023, 4025, 4026, 4028, 4029, 4031, 4032, 4034, 4035, 4036, 4037, 4038, 4040, 4052, 4055, 4056, 4060, 4061, 4066, 4067, 4070, 4077, 4078, 4080, 4081, 4082, 4083, 4084, 4085, 4086, 4087, 4088, 4089, 4090, 4102, 4105, 4106, 4108, 4109, 4110, 4114, 4115, 4117, 4118, 4119, 4128, 4129, 4132, 4136, 4137, 4142, 4147, 4159, 4163, 4168, 4170, 4171, 4172, 4173, 4175, 4182, 4183, 4186, 4188, 4192, 4194, 4199, 4208, 4225, 4226, 4227, 4228, 4232, 4239, 4240, 4258, 2557, 2988, 2993, or 2585.

20. (canceled)

21. A prime editing system comprising:
a. the prime editing guide RNA (PEgRNA) of claim 1, or a nucleic acid encoding the PEgRNA; and
b. a nick guide RNA (ngRNA) comprising at its 3′ end nucleotides 5-20 of any one of SEQ ID NOs: 41, 60, 61, 62, 63, 64, 65, 66, 69, 71, 2045, 2046, 2047, 2048, 2049, 2050, 2051, 2052, 2053, 2054, 2055, 2056, 2057, 2058, 2059, 2060, 2061, 2062, 2063, 2064, 2065, 2066, 2067, 2068, 2069, 2070, 2071, 2072, 2073, 2074, 2075, 2076, 2077, 2078, 2079, 2080, 2081, 2082, 2083, 2084, 2085, 2086, 2087, 2088, 2089, 2090, 2091, 2092, 2093, 2094, 2095, 2096, 2423, 2424, 2425, 2426, 2427, 2428, 2429, 2430, 2431, 2432, 2433, 2434, 2435, 2436, 2437, 2438, 2439, 2440, 2441, 2442, 2443, or 2444 and a gRNA core capable of binding to a Cas9 protein, or a nucleic acid encoding the ngRNA.

22. (canceled)

23. The prime editing system of claim 21, wherein the spacer of the ngRNA comprises at its 3′ end nucleotides 4-20, 3-20, 2-20, or 1-20 of any one of SEQ ID NOs: 41, 60, 61, 62, 63, 64, 65, 66, 69, 71, 2045, 2046, 2047, 2048, 2049, 2050, 2051, 2052, 2053, 2054, 2055, 2056, 2057, 2058, 2059, 2060, 2061, 2062, 2063, 2064, 2065, 2066, 2067, 2068, 2069, 2070, 2071, 2072, 2073, 2074, 2075, 2076, 2077, 2078, 2079, 2080, 2081, 2082, 2083, 2084, 2085, 2086, 2087, 2088, 2089, 2090, 2091, 2092, 2093, 2094, 2095, 2096, 2423, 2424, 2425, 2426, 2427, 2428, 2429, 2430, 2431, 2432, 2433, 2434, 2435, 2436, 2437, 2438, 2439, 2440, 2441, 2442, 2443, or 2444.

24.-25. (canceled)

26. The prime editing system of claim 21, wherein the ngRNA comprises SEQ ID NOs: 2257, 2258, 2259, 2260, 2261, 2262, 2263, 2264, 2265, 2266, 2267, 2268, 2269, 2270, 2271, 2272, 2273, 2274, 2275, 2276, 2277, 2278, 2279, 2280, 2281, 2282, 2283, 2284, 2285, 2286, 2287, 2288, 2289, 4410, 4411, 4412, 4413, 4414, 4415, 4416, 4417, 4418, 4419, 4420, 4421, or 4422.

27.-95. (canceled)
